(19) United States
(12) Patent Application Publication
(10) Pub. No.: US 2025/0041306 A1
(43) Pub. Date: Feb. 6, 2025

(54) TARGETED PROTEASE DEGRADATION (TED) PLATFORM

(71) Applicant: EUBULUS BIOTHERAPEUTICS INC., Jiaxing, Zhejiang (CN)

(72) Inventors: Sheldon CAO, Jiaxing, Zhejiang (CN); Xiaolei WANG, Jiaxing, Zhejiang (CN)

(21) Appl. No.: 18/699,857

(22) PCT Filed: Oct. 9, 2022
(86) PCT No.: PCT/CN2022/124206

§ 371 Date: Apr. 9, 2024

(30) Foreign Application Priority Data
Oct. 9, 2021 (CN) 202111176342.5

(51) Int. Cl. A61K 31/519; A61K 31/506; A61K 47/54; C07D 401/14; C07D 487/04

(52) U.S. Cl. CPC A61K 31/519; A61K 31/506; A61K 47/54; C07D 401/14; C07D 487/04

(57) ABSTRACT

A targeted protease degradation (TED) platform that can be a conjugate of target molecule-linker-E3 ligase ligand as shown in formula I, i.e., R_{T}-L1-R_{E3}, wherein R_{T} is a monovalent group of the target molecule; R_{E3} is a monovalent group of the E3 ligase ligand; L1 is a linker linking A and B; and L1 is as shown in formula III below: —W¹-L2-W²—(II).

### TECHNICAL FIELD

The present invention belongs to biomedicine, specifically, relates to a targeted protease degradation (TED) platform.

### BACKGROUND

Expression level of proteins is regulated on three basic levels according to modern molecular biology. Firstly, at the DNA level, it can be achieved by inactivating the target protein DNA through gene knock-out. Secondly, at the mRNA level, it can be achieved by binding to the mRNA of the target protein through small molecule RNA, thereby inhibiting the translation and expression of mRNA. Thirdly, at the protein level, it can be achieved by modifying the target protein post translation, such as methylation, phosphorylation, glycosylation, etc, thereby regulating the amount and activity of the target protein.

In terms of the overall development of drug research and development, both small molecule and macromolecule drug forms have their own advantages and disadvantages. For example, the development of small molecule drugs has been facing crucial challenges such as how to maintain drug concentration in the body and drug resistance. Some target sites have shapes that are adverse to the design of small molecule drugs, and thus become non-drugable targets. For these targets, no effective regulatory methods have yet been found. Although, compared to small molecules, monoclonal antibodies have the advantages of high affinity and high selectivity, and easy to develop highly effective and highly selective drugs, the biggest drawback thereof is that they cannot penetrate cell membranes and therefore cannot act on intracellular targets. Antibody-drug conjugates (ADC) utilize endocytic antibodies to provide targetability and serve as carriers to deliver super toxin drugs to the targeted site. The bottleneck encountered in the development of ADC drugs is that the therapeutic window is not wide enough. In addition to the side effects caused by the antibody itself, the super toxins will fall off before reaching the targeting site due to the heterogeneity of coupling, and causing serious side effects. In addition, normal physiological function of ubiquitin-proteasome system is responsible for cleaning up denatured, mutated or harmful proteins in cells.

In summary, there is an urgent need in the art to develop a compound that is able to degrade target proteins more efficiently and re-usably so as to treat related diseases.

### SUMMARY OF THE INVENTION

The purpose of the present invention is to provide a compound that is able to degrade target proteins more efficiently and re-usably so as to treat related diseases.

In the first aspect of the present invention, provided is a conjugate of formula I, and a pharmaceutically acceptable salt thereof,

R_{T}-L1-R_{E3} (I)
wherein
(a) R_{E3} is a moiety of E3 Ligase Ligand;
(b) R_{T} is a moiety of target molecule;
(c) L1 is a linker connecting the moieties of R_{E3} and R_{T}, and L1 is as shown in formula II;

—W¹-L2-W²— (II)
wherein
W¹ and W² are each independently —(W)ₛ—;
W is each independently selected from the group consisting of null(bond), —C(R^{b})₂—, —O—, —S—, —N(R^{a})—, —C(O)—, —SO₂—, —SO—, —PO₃—, —C(R^{b})═C(R^{b})—, —C≡C—, NR, substituted or unsubstituted C3-8 cycloalkyl, substituted or unsubstituted 4 to 10 membered heterocycloalkyl, substituted or unsubstituted C6-10 aryl, substituted or unsubstituted 5 to 10 membered heteroaryl;
s=0, 1, 2, 3, or 4;
L2 is of formula III,

-(M^{L})ₒ- (III)
wherein
M^{L} is each independently M, M^{T} or M^{N};
wherein
is an integer selected from 5 to 50;
M is each independently a divalent group selected from the group consisting of: —C(R^{b})₂—, —O—, —S—, —N(R^{a})—, —C(O)—, —SO₂—, —SO—, —PO₃—, —C(R^{b})═C(R^{b})—, —C≡C—, substituted or unsubstituted C₃₋₈ cycloalkyl, substituted or unsubstituted 4 to 10 membered heterocycloalkyl, substituted or unsubstituted C₆₋₁₀ aryl, substituted or unsubstituted 5 to 10 membered heteroaryl, and amino acid residue;
M^{N} is each independently a divalent group selected from the group consisting of: —N(R′)—, —N(4 to 10 membered heterocycloalkyl containing N(R′) as ring atom)-, 4 to 10 membered heterocycloalkyl containing N(R′) as ring atom, —C(R^{b})₂— substituted with at least one —N(R^{b})R′ (preferably, —NHR′), C₃₋₈ cycloalkyl, 4 to 10 membered heterocycloalkyl, C₆₋₁₀ aryl, or 5 to 10 membered heteroaryl;
M^{T} is each independently a divalent group selected from the group consisting of: —N(R″)—, —N(4 to 10 membered heterocycloalkyl containing N(R″) as ring atom)-, 4 to 10 membered heterocycloalkyl containing N(R″) as ring atom, —C(R^{b})₂— substituted with at least one —N(R^{b})R″ (preferably, —NHR″), C₃₋₈ cycloalkyl, 4 to 10 membered heterocycloalkyl, C₆₋₁₀ aryl, and 5 to 10 membered heteroaryl;
R is R′ or R″;
R′ is each independently selected from the group consisting of H, C₁₋₆ alkyl, OH, SH, —COO—C₁₋₆ alkyl, —OC(O)—C₁₋₆ alkyl, and amino protecting group;
R″ is —W³-L^{T1}-W^{P1}—(R_{P})_{q1};
subscript q1>0 (preferably, q1=1);
W^{P1} is null, —S—S— or

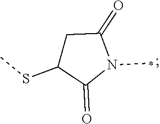

wherein * indicates the part connected with L^{T1}; preferably, W^{P1} is —S—S— or

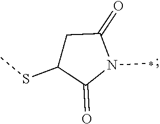

R_{P} is —W⁴—R_{P1}; W⁴ is null or —(W″)ₛ₁—W^{P2}—(W″)ₛ₂—; wherein subscript s1 and s2 are each independently 0, 1, 2, 3 or 4, W^{P2} is null, NH, —C(R^{b})(NR^{a})— (such as —CH(—NH₂)—), —N(R′″)— or —C(R^{b})(NH(R′″))—;
R′″ is —W⁵-L^{T2}-W⁶-L^{T3}-R_{P2};
L^{T1} is -(M′)ₜ₁—W_{Y}-(M′)ₕ₂-;
L^{T2} is -(M′)ₜ₃-;
L^{T3} is -(M′)ₜ₄-;
subscripts t1, t2, t3 and t4 are each independently 0, 1, 2, 3, 4, 5, 6, 7, 8, 9 or 10 (preferably, t1, t2, t3 and t4 are each independently 0, 1, 2 or 3);
M′ is each independently selected from the group consisting of —C(R^{b})₂—, —O—, —S—, —N(R^{a})—, —C(O)—, —SO₂—, —SO—, —PO₃—, substituted or unsubstituted C1-10alkylene, —(CH₂CH₂O)₁₋₁₀—, amino acid residue, substituted or unsubstituted C3-8cycloalkyl, substituted or unsubstituted 4-10 membered heterocycloalkyl, substituted or unsubstituted C6-10aryl, and substituted or unsubstituted 5-10 membered heteroaryl; and any one or two M′ is W_{X};
W_{X} is a moiety of hydrophilic bivalent linker;
W_{Y} is null or a moiety of bivalent linker that is cleavable at the cell surface or in the cytoplasm;
W³ is —(W′)ₛ₃—; wherein subscript s3=0, 1 or 2;
W⁵ is —(W′)ₛ₄—; wherein subscript s4=0, 1 or 2;
W⁶ is

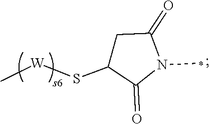

or —(W″)ₛ₆—; wherein subscript s6=0, 1, 2, 3 or 4;
W′ are each independently a divalent group selected from the group consisting of —C(R^{b})₂—, —O—, —S—, —N(Ra)—, —C(O)—, —SO₂—, —SO—, —PO₃—, amino acid residue, substituted or unsubstituted C3-8 cycloalkyl, substituted or unsubstituted 4 to 10 membered heterocycloalkyl, substituted or unsubstituted C6-10 aryl, and substituted or unsubstituted 5 to 10 membered heteroaryl;
W″ are each independently a divalent group selected from the group consisting of —C(R^{b})₂—, —O—, —S—, —N(R^{a})—, —C(O)—, —SO₂—, —SO—, —PO₃—, amino acid residue, substituted or unsubstituted C3-8 cycloalkyl, substituted or unsubstituted 4 to 10 membered heterocycloalkyl, substituted or unsubstituted C6-10 aryl, and substituted or unsubstituted 5 to 10 membered heteroaryl;
R_{P1} and R_{P2} are each independently the same or different polypeptide element or target molecule T; preferably, R_{P1} and R_{P2} are each independently different polypeptide element or target molecule T;
R^{a} is each independently selected from the group consisting of H, OH, SH, substituted or unsubstituted C₁₋₆ alkyl, amino protecting group, 4 to 10 membered heterocycloalkyl containing N(RC) as ring atom;
R^{b} is each independently selected from the group consisting of H, halogen, OH, SH, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₁₋₆alkanoyl (—C(O)—C₁₋₆alkyl), carboxyl, —COO—C₁₋₆alkyl, —OC(O)—C₁₋₆alkyl; or, two R^{b} on the same atom together with the carbon to which they are attached form substituted or unsubstituted C₃₋₈cycloalkyl, substituted or unsubstituted 4 to 10 membered heterocycloalkyl;
R^{c} is each independently selected from the group consisting of H, OH, SH, substituted or unsubstituted C₁₋₆ alkyl, and amino protecting group;
unless otherwise specified, said substituted means that one or more (such 1, 2, or 3) hydrogen atoms in the group are substituted with substituents selected from the group consisting of halogen (preferably, F, Cl, Br or I), cyano(CN), oxo (═O), thio (═S), C₁₋₆alkyl, C₁₋₆haloalkyl, C₂₋₆alkenyl, C₂₋₆alkynyl, C₁₋₆alkoxy, C₁₋₆alkanoyl (C₁₋₆alkyl-C(O)—), —COO—C₁₋₆alkyl, —OC(O)—C₁₋₆alkyl, NH₂, NH(C₁₋₆alkyl), N(C₁₋₆alkyl)₂.

In another preferred embodiment, when W^{P1} is null or

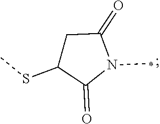

W_{Y} is a moiety of bivalent linker that is cleavable at the cell surface or in the cytoplasm.

In another preferred embodiment, the moiety of bivalent linker that is cleavable at the cell surface or in the cytoplasm refers to a bivalent linker moiety that is capable of cleavage in the acidic environment on the cell surface or in the cytoplasm or being cleaved specifically by a GSH enzyme.

In another preferred embodiment, the moiety of bivalent linker that is cleavable at the cell surface or in the cytoplasm is a bivalent linker moiety composed of two or more structural fragments selected from the group consisting of:

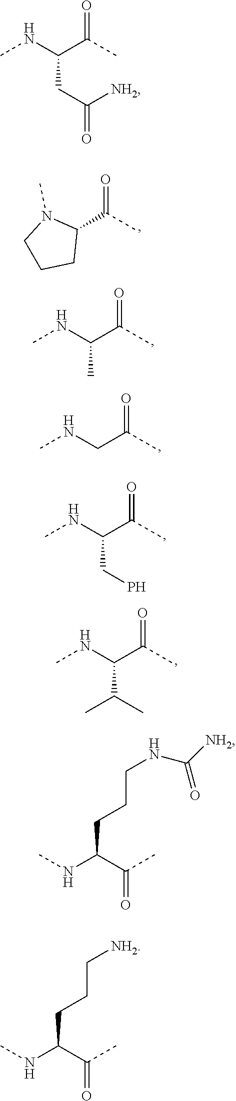

In another preferred embodiment, the moiety of bivalent linker that is cleavable at the cell surface or in the cytoplasm is selected from the group consisting of:

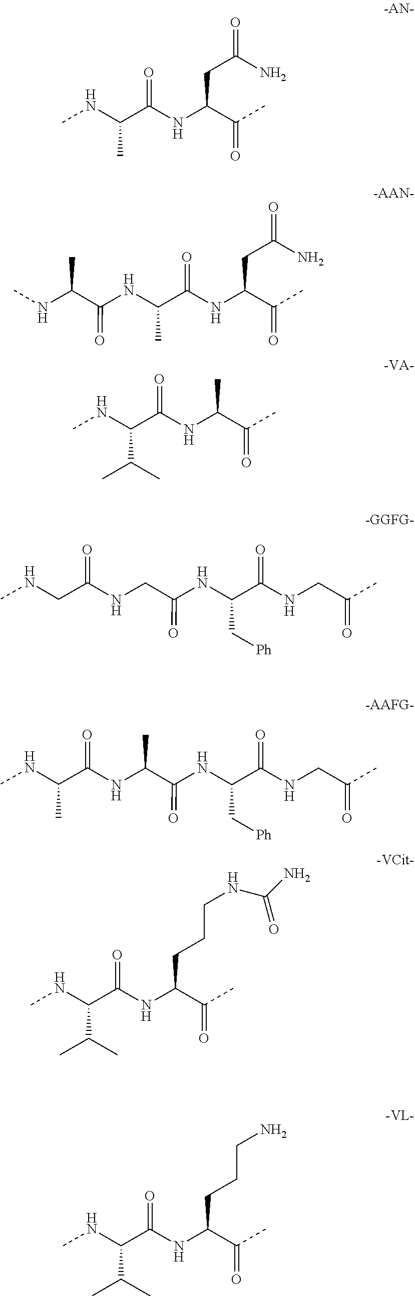

In another preferred embodiment, t1+t2≤4; preferably, t1+t2=3 or 4.

In another preferred embodiment, W^{P2} is null, —C(R^{b})(NR^{a})— (such as —CH(—NH₂)—) or —CH(NH(R′″))—.

In another preferred embodiment, W⁴ is null, —NH—CH(COOH)—CH₂—, —NH—C(O)—CH(NH₂)—CH₂—, or —NH—C(O)—CH(NH(R′″))—CH₂—.

In another preferred embodiment, the moiety of hydrophilic bivalent linker refers to a bivalent linker moiety containing one or more groups on the main chain or side chain selected from the group consisting of: —(CH₂CH₂O)—, —SO₃H, —PO₃H₂, —COOH.

In another preferred embodiment, the moiety of hydrophilic bivalent linker or W_{X} is selected from the group consisting of:

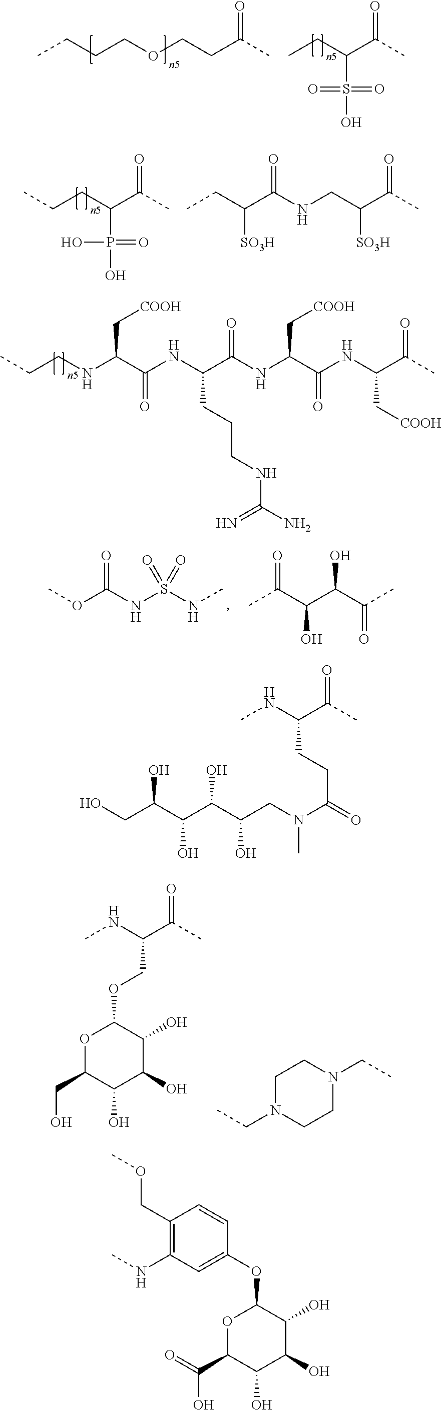

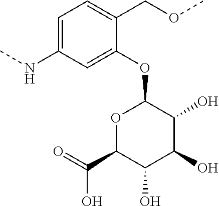

wherein n5 is an integer selected from 0 to 30 (preferably, n5=0, 1, 2, 3, 4, 5, 6, 7, 8, 9 or 10).

In another preferred embodiment, W³ is null, —C(O)— or —OC(O)—.

In another preferred embodiment, W⁵ is null, —C(O)— or —OC(O)—.

In another preferred embodiment, in L^{T1}, L^{T2} and L^{T3}, there is an M′ that is Wx.

In another preferred embodiment, W is not NR.

In another preferred embodiment, W is each independently selected from the group consisting of null, —C(R^{b})₂—, —O—, —S—, —N(Ra)—, —C(O)—, —SO₂—, —SO—, —PO₃—, —C(R^{b})═C(R^{b})—, —C≡C—, substituted or unsubstituted C3-8 cycloalkyl, substituted or unsubstituted 4 to 10 membered heterocycloalkyl, substituted or unsubstituted C6-10 aryl, substituted or unsubstituted 5 to 10 membered heteroaryl; and s=1 or 2.

In another preferred embodiment, W¹ and W² are each independently —N(R^{a})—C(O)—, —C(O)—N(R^{a})— or —C≡C—.

In another preferred embodiment, W¹ is —N(Ra)—C(O)—, or —C(O)—N(R^{a})—; and W² is —C≡C—.

In another preferred embodiment, one of R_{P1} and R_{P2} is polypeptide element, and the other is target molecule T.

In another preferred embodiment, R_{P1} and R_{P2} are the same or different polypeptide element.

In another preferred embodiment, R_{P1} and R_{P2} are the same or different target molecule T.

In another preferred embodiment, R_{P1} and R_{P2} are each independently selected from the group consisting of:

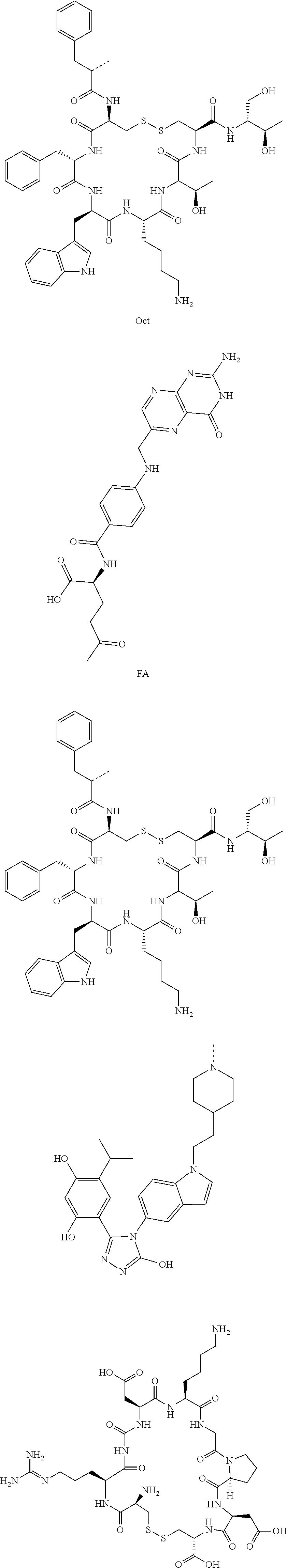

In another preferred embodiment, there is no —O—O— in L2.

In another preferred embodiment, in L2, at least one of M^{L} is M^{T} or M^{N}.

In another preferred embodiment, in L2, when two or more of M^{L} is M^{T} or M^{N}, L2 comprises M^{T} and M^{N}, or L2 comprises only M^{T}, or L2 only comprises M^{N}.

In another preferred embodiment, in L2, at least one of M^{L} is M^{N}.

In another preferred embodiment, in L2, at least one of M^{L} is M^{T}.

In another preferred embodiment, in L2, 1, 2 or 3 of M^{L} are each independently M^{T} or M^{N}.

In another preferred embodiment, in L2, 1, 2 or 3 of M^{L} are each independently M^{N}.

In another preferred embodiment, in L2, 1, 2 or 3 of M^{L} are each independently M^{T}.

In another preferred embodiment, L2 is L5, and L5 is of formula IIIc;

-(M)ₒ₁-(M′)-(M)ₒ₂- (IIIc)
wherein
M′ is each independently M^{T} or M^{N};
M, M^{T} and M^{N} are as defined in formula I;
o1 and o2 are each independently integers selected from 1 to 50, and 4≤o1+o2≤49.

In another preferred embodiment, L2 is L6, and L6 is of formula IIIa;

-(M)ₒ₁-(M^{N})-(M)ₒ₂- (IIIa)
wherein
M, and M^{N} are defined as above;
o1 and o2 are each independently integers selected from 1 to 50, and 4≤o1+o2≤49.

In another preferred embodiment, o1 and o2 are each independently 1, 2, 3, 4, 5, 6, 7 or 8.

In another preferred embodiment, o1 is 1, or 2, and o2 is 1, 2, 3, 4, 5, 6 or 7.

In another preferred embodiment, in L6, M is each independently selected from the group consisting of —CH₂—, —CH(C₁₋₄alkyl)-, —CH(NH₂)—, —O—, —NH—, —N(C₁₋₄alkyl)-,

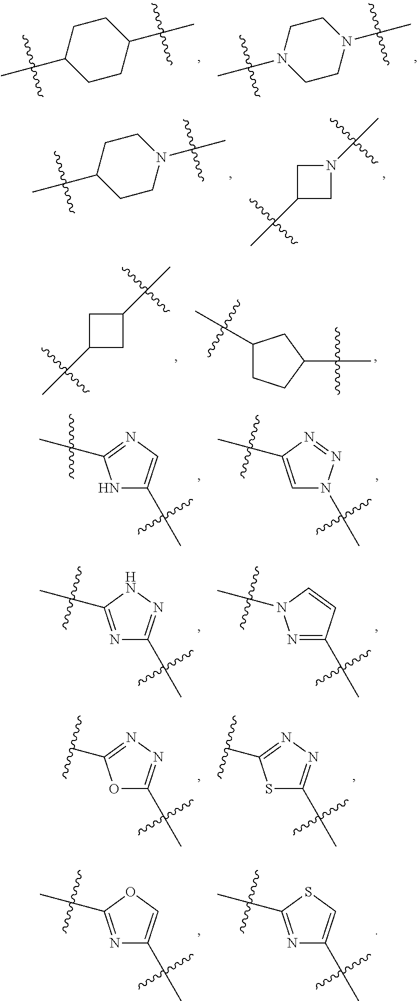

In another preferred embodiment, the conjugate is of formula IV;

R_{T}—W¹-L6-W²—R_{E3} (IV)
wherein, L6, W¹, W², R_{T} and R_{E3} are defined as in formula I.

In another preferred embodiment, L2 is L7, and L7 is of formula IIIb;

-(M)ₒ₁-(M^{T})-(M)ₒ₂- (IIIb)
wherein M, and M^{T} are defined as above;
o1 and o2 are each independently integers selected from 1 to 50, and 4≤o1+o2≤49.

In another preferred embodiment, o1 and o2 are each independently 1, 2, 3, 4, 5, 6, 7 or 8.

In another preferred embodiment, the conjugate is of formula V;

R_{T}—W¹-L7-W²—R_{E3} (V);

Wherein, L7, W¹, W², R_{T} and R_{E3} are as defined in formula I.

In another preferred embodiment, the conjugate is of formula 1-1, 1-2, 1-3, 2 or 3;

R_{T}—W¹-L5-W^{b}—C≡C—R_{E3} (1-1);

R_{T}—W¹-L5-CO—R_{E3} (1-2);

R_{T}—W¹-L5-CONH—R_{E3} (1-3);

R_{T}—W^{a}—Cr¹—W^{a}—Cr²-L5-W²—R_{E3} (2)

R_{T}-Ar1-L5-W²—R_{E3} (3)
wherein
Ar1 is −5 or 6 membered heteroaryl containing nitrogen atom-;
Cr¹ is null, or C₄₋₇cycloalkyl that is unsubstituted or substituted with C₁₋₄alkyl, or 4 to 6 membered heterocyclyl that is unsubstituted or substituted with C₁₋₄alkyl;
Cr² is 4 to 6 membered heterocyclyl containing nitrogen atom that is unsubstituted or substituted with C₁₋₄alkyl, and at least one of nitrogen heteroatom in Cr² is attached with L7;
the definition of W^{a} and W^{b} is the same as that of W; and W, W¹, W², R_{T}, R_{E3} and L5 are defined as above.

In another preferred embodiment, the conjugate is of formula 1a-1, 1a-2, 1a-3, 2a or 3a;

R_{T}—W¹-L6-W^{b}—C≡C—R_{E3} (1a-1);

R_{T}—W¹-L6-CO—R_{E3} (1a-2);

R_{T}—W¹-L6-CONH—R_{E3} (1a-3);

R_{T}—W^{a}—Cr¹—W^{a}—Cr²-L6-W²—R_{E3} (2a)

R_{T}-Ar1-L6-W²—R_{E3} (3a)

wherein
Ar1, Cr¹, Cr², W^{a}, W^{b}, W¹, W², R_{T}, R_{E3} and L6 are defined as above.

In another preferred embodiment, the conjugate is of formula 1b-1, 1b-2, 1b-3, 2b or 3b;

R_{T}—W¹-L7-W^{b}—C≡C—R_{E3} (1b-1);

R_{T}—W¹-L7-CO—R_{E3} (1b-2);

R_{T}—W¹-L7-CONH—R_{E3} (1b-3);

R_{T}—W^{a}—Cr¹—W^{a}—Cr²-L7-W²—R_{E3} (2b)

R_{T}-Ar1-L7-W²—R_{E3} (3b)
wherein
Ar1 is 5 or 6 membered heteroaryl containing nitrogen atom;
Cr¹ is null, or C₄₋₇cycloalkyl that is unsubstituted or substituted with C₁₋₄alkyl, or 4 to 6 membered heterocyclyl that is unsubstituted or substituted with C₁₋₄alkyl;
Cr² is 4 to 6 membered heterocyclyl containing nitrogen that is unsubstituted or substituted with C₁₋₄alkyl, and at least one of nitrogen heteroatom in Cr² is attached with L7;
the definition of W^{a} and W^{b} is the same as that of W; and W, W¹, W², R_{T}, R_{E3} and L7 are defined as above.

In another preferred embodiment, L2 is L8, and L8 is of formula IIId;

-(M)ₒ₃- (IIId)

Wherein M is defined as above (preferably, M is CH2), o3 is 1, 2, 3, 4 or 5.

In another preferred embodiment, the conjugate is of R_{T}—W¹-L8-W²—R_{E3}; wherein R_{T}, W¹, L8, W², and R_{E3} are defined as above. Preferably, W¹ is W^{a}—Cr¹—Cr² (more preferably, is NH—Cr¹—Cr²), Cr¹ and Cr² are defined as above.

In another preferred embodiment, when the heterocycloalkyl (such as 4 to 10 membered heterocycloalkyl) is a divalent group, the 4 to 10 membered heterocycloalkyl includes

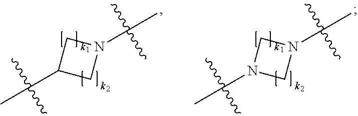

wherein k1 and k2 are each independently 0, 1, 2 or 3; preferably, the 4 to 10 membered heterocycloalkyl is selected from the group consisting of:

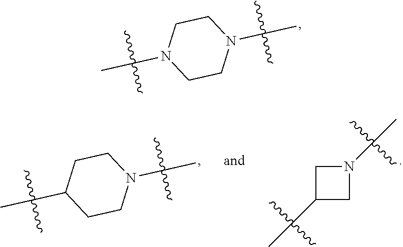 [SMILES: *N1CCN(*)CC1 ; *C1CCN(*)CC1 ; *C1CN(*)C1]

In another preferred embodiment, when the cycloalkyl (such as C₃₋₈cycloalkyl) is a divalent group, the cycloalkyl (such as C₃₋₈cycloalkyl) includes

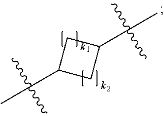

wherein k1 and k2 are each independently 1, 2 or 3; preferably, the C₃₋₈cycloalkyl is selected from the group consisting of:

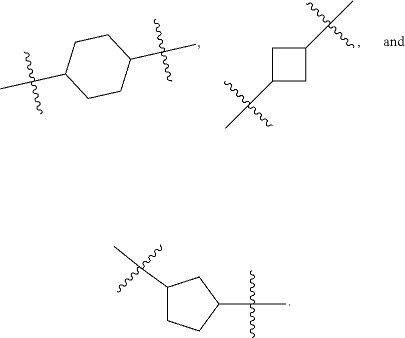 [SMILES: *C1CCC(*)CC1 ; *C1CC(*)C1 ; *C1CCC(*)C1]

In another preferred embodiment, when the heteroaryl (such as 5 to 10 membered heteroaryl) is a divalent group, the heteroaryl (such as 5 to 10 membered heteroaryl) is

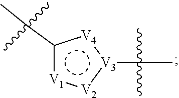

wherein V₁, V₂ and V₄ are each independently selected from the group consisting of: —O—, —S—, —N═, —NH—, —CH═, —CH₂—; V₃ is selected from the group consisting of: —N═, —CH═; preferably, the 5 to 10 membered heteroaryl is selected from the group consisting of:

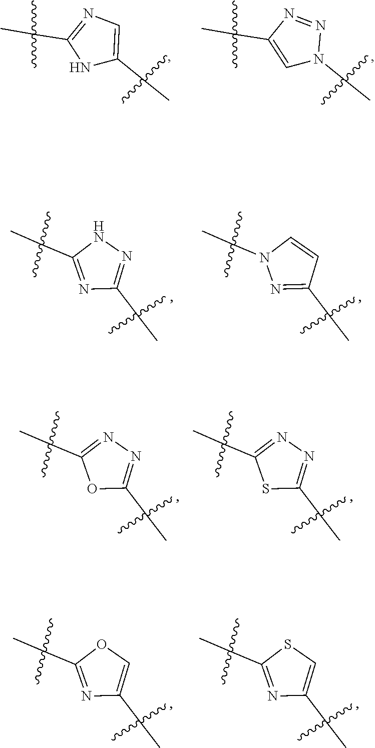

In another preferred embodiment, M is each independently selected from the group consisting of —CH₂—, —CH(C₁₋₄alkyl)-, —CH(NH₂)—, —O—, —NH—, —N(C₁₋₄alkyl)-,

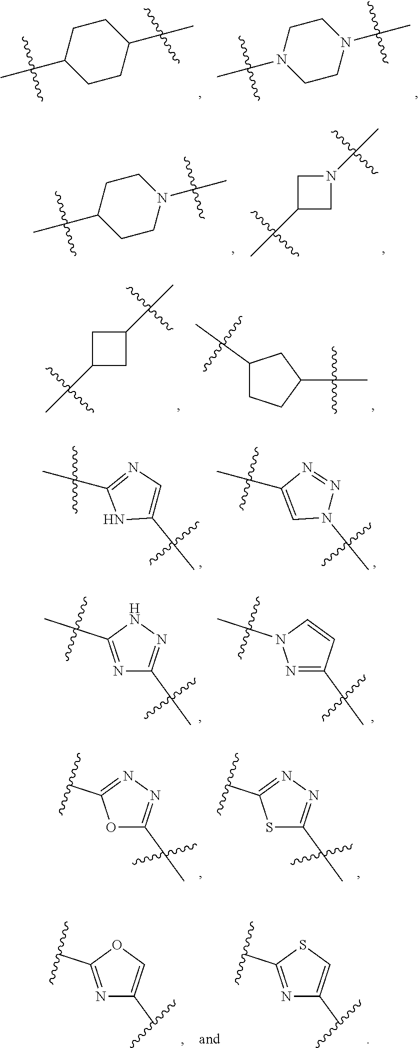

In another preferred embodiment, when the 4 to 10 membered heterocycloalkyl containing N(R) as ring atom is a divalent group, the 4 to 10 membered heterocycloalkyl containing N(R) as ring atom is selected from the group consisting of:

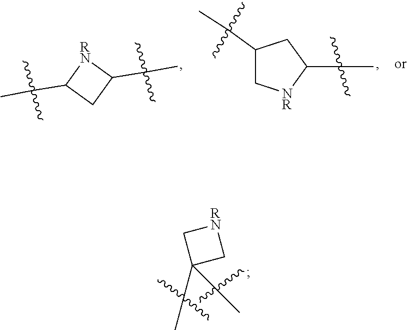

wherein, R is R′ or R″.

In another preferred embodiment, M^{T} is each independently selected from the group consisting of —N(R″)—, —C(R^{b})(NHR″)—,

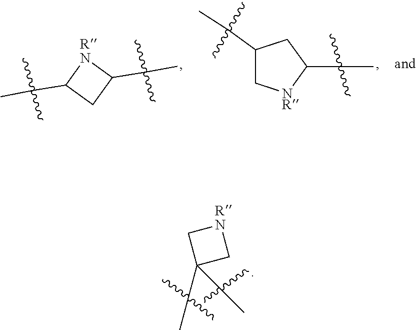

In another preferred embodiment, M^{T} is the following divalent group: —N(R″)—. In another preferred embodiment, M^{N} is each independently selected from the group consisting of —N(R′)—, —C(R^{b})(NHR′)—,

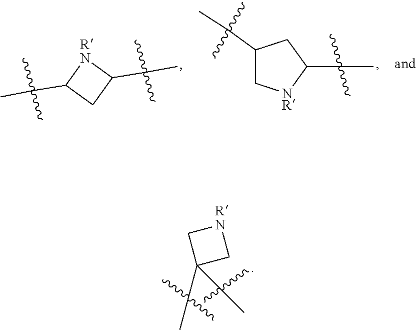

In another preferred embodiment, M^{N} is the following divalent group: —N(R′)—.

In another preferred embodiment, M is each independently selected from the group consisting of O, C(R^{b})₂; preferably, wherein R^{b} is each independently H or C₁₋₆alkyl (such as methyl).

In another preferred embodiment, W is selected from the group consisting of null, —C(R^{b})₂—, —O—, —S—, —N(R^{a})—, —C(O)—, —SO₂—, —SO—, —PO₃—, —C(R^{b})═C(R^{b})—, —C≡C—; or, W is substituted or unsubstituted groups selected from the group consisting of

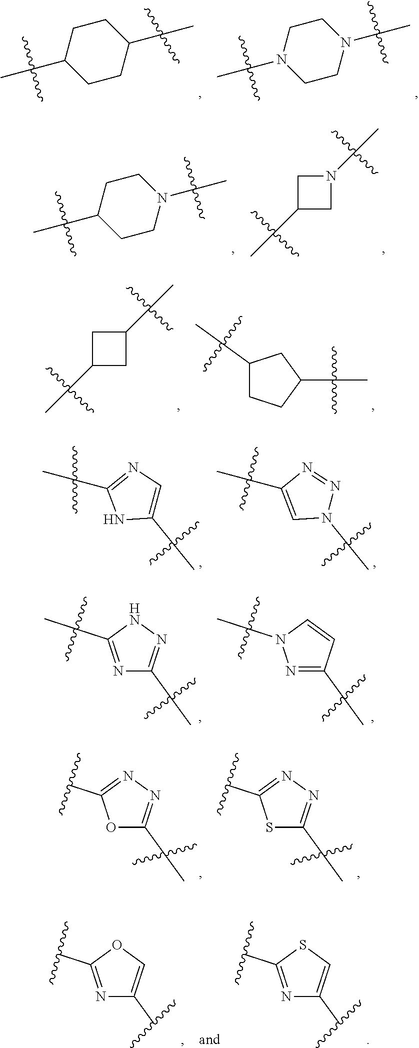

In another preferred embodiment, R^{a} is each independently H or C₁₋₆alkyl (such as methyl).

In another preferred embodiment, R^{b} is each independently H or C₁₋₆alkyl (such as methyl).

In another preferred embodiment, RC is each independently H or C₁₋₆alkyl (such as methyl).

In another preferred embodiment, L3 is -(M^{a})ₚ-; wherein M^{a} is defined as M, p is an integer selected from 1 to 50.

In another preferred embodiment, p=1, 2, 3, 4, 5, 6, 7, 8, 9, 10, 11, 12, 13, 14, or 15.

In another preferred embodiment, M^{a} are each independently divalent groups selected from the following group of: —C(R^{b})₂—, —O—, —S—, —N(R^{a})—, —C(O)—, —SO₂—, —SO—, —PO₃—, —C(R^{b})═C(R^{b})—, —C≡C—, substituted or unsubstituted —C3-8cycloalkyl-, substituted or unsubstituted −4 to 10 membered heterocycloalkyl, substituted or unsubstituted —C6-10 aryl, substituted or unsubstituted 5 to 10 membered heteroaryl, and amino acid residue.

In another preferred embodiment, —W³-L3-W⁴—R_{P} is selected from the group consisting of

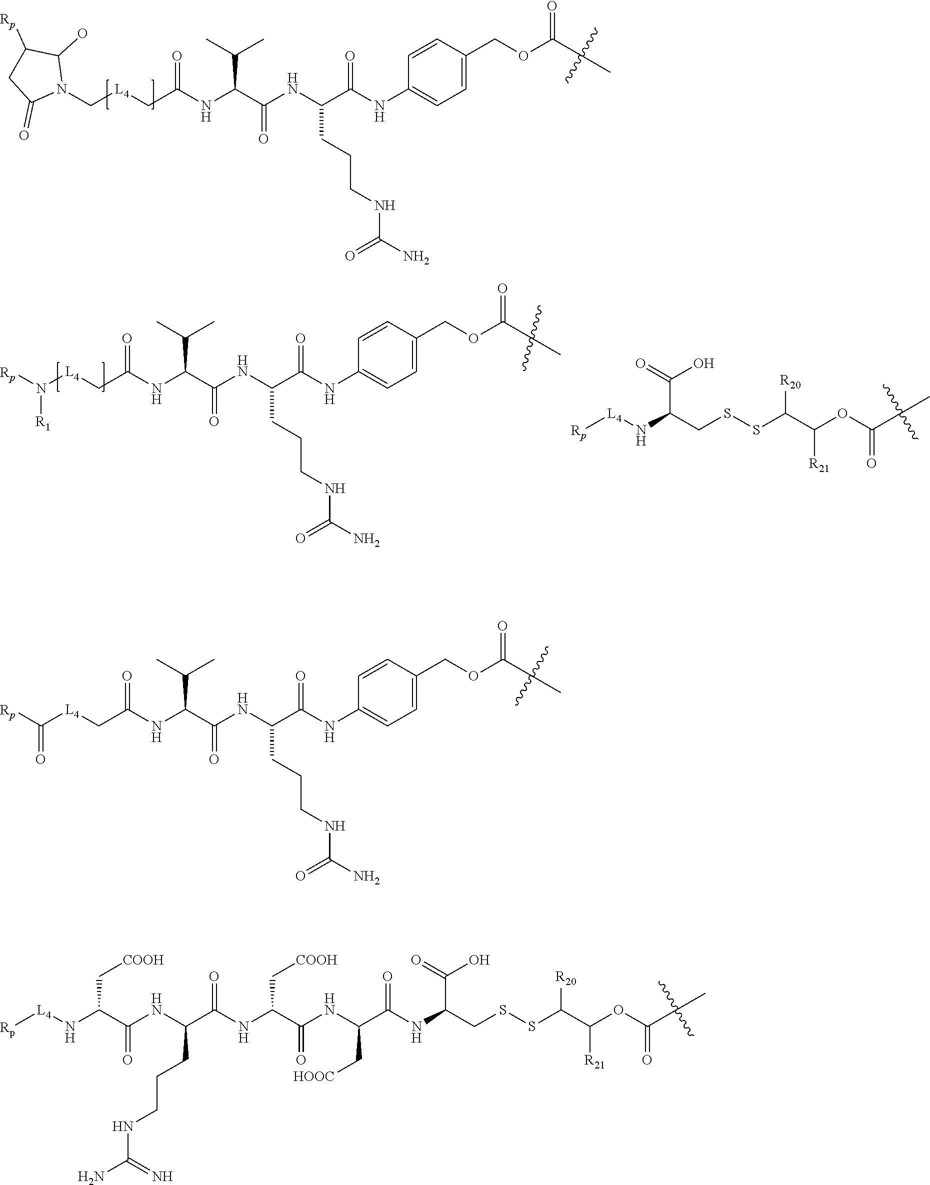

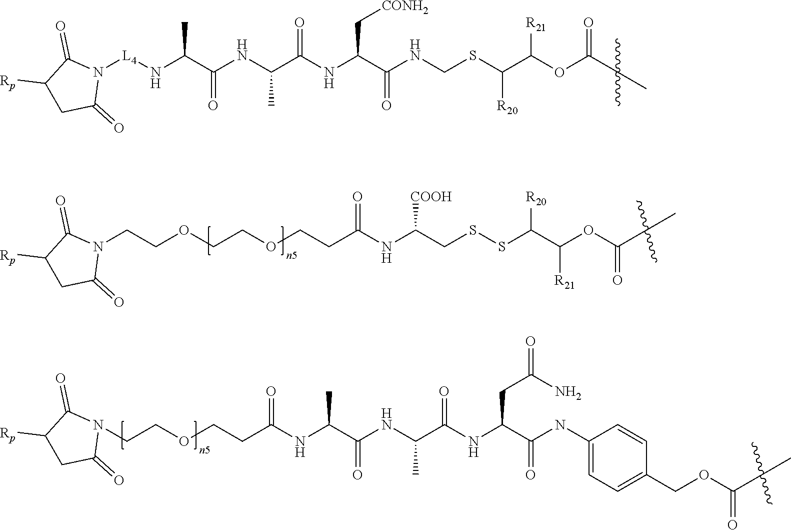

wherein L4 is -(M)_{q}-, wherein M is defined as in L2;
q is an integer of 1 to 50 and q is less than p (preferably, q=an integer selected from 0 to 30; more preferably, q=0, 1, 2, 3, 4, 5, 6, 7, 8, 9, or 10), n5 is an integer selected from 0 to 30 (preferably, n5=0, 1, 2, 3, 4, 5, 6, 7, 8, 9, or 10); R₂₀ and R₂₁ are each independently selected from the group consisting of —H, -Me, -Et, -nPr, iPro, cPro. In another preferred embodiment, the conjugate is a conjugate selected from Group 1, Group 2 and Group 3; In another preferred embodiment, the conjugate is a conjugate selected from Group 1a, Group 2a and Group 3a.

In another preferred embodiment, the conjugate is selected from the group consisting of:

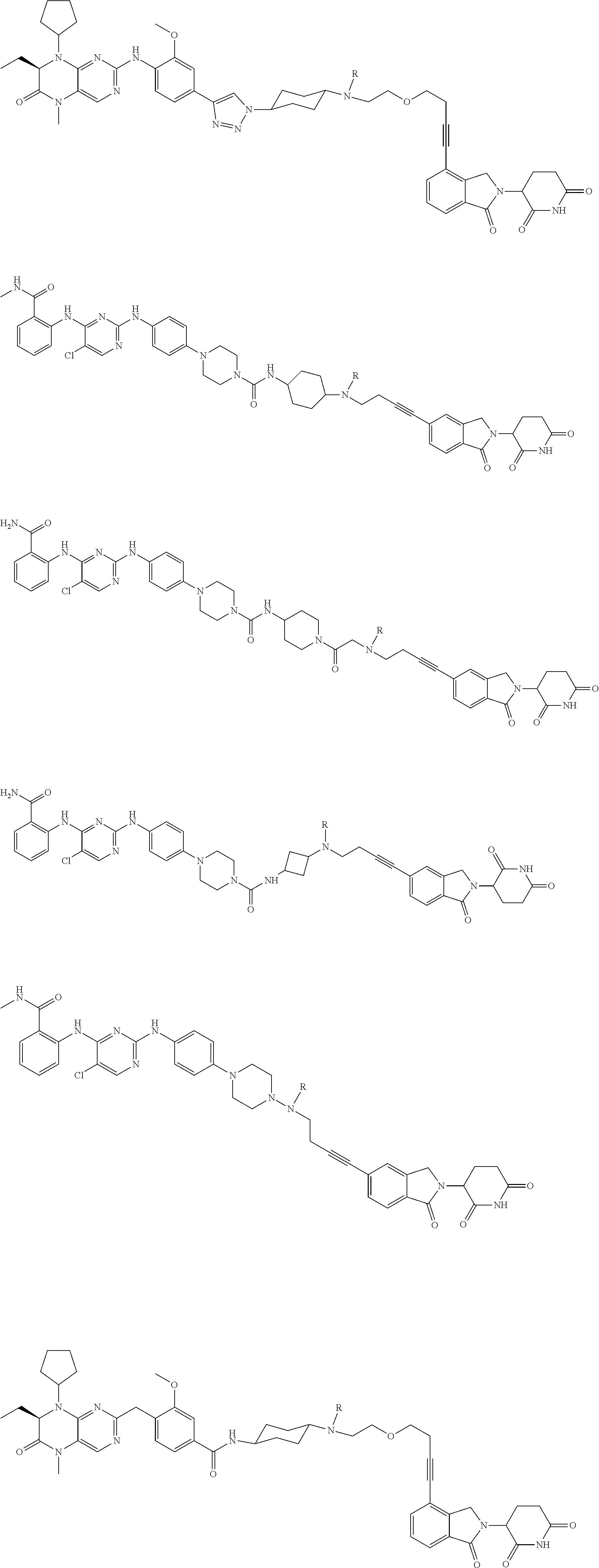

In another preferred embodiment, the conjugate is a conjugate selected from Group 1, Group 2 and Group 3; wherein R and R¹ are R″ (i.e. R and R¹ are each independently —W³-L3-W⁴—(R_{P})_{q}).

In another preferred embodiment, the conjugate of formula I is a conjugate of formula X.

R_{P}—(W⁴-L3-W³—R_{TED})ₜ (X)
wherein t=1/q (preferably, t=1 to 8);
R_{P} is defined as above, preferably R_{P} is polypeptide element, more preferably, antibody;
R_{TED}—W⁴-L3-W³— is the remaining part of the conjugate of formula I after losing the Rp group.

In another preferred embodiment, R_{TED} is a monovalent group derived from specific compounds of conjugates in Tables A1, and A2, conjugates in Group 1a, Group 2a and Group 3a (wherein, said derived means a monovalent group formed by the specific compounds shown in Tables A1 and A2 losing a hydrogen from NH on the main chain or the branched chain of the linker group).

In another preferred embodiment, the conjugate is selected from the group consisting of:

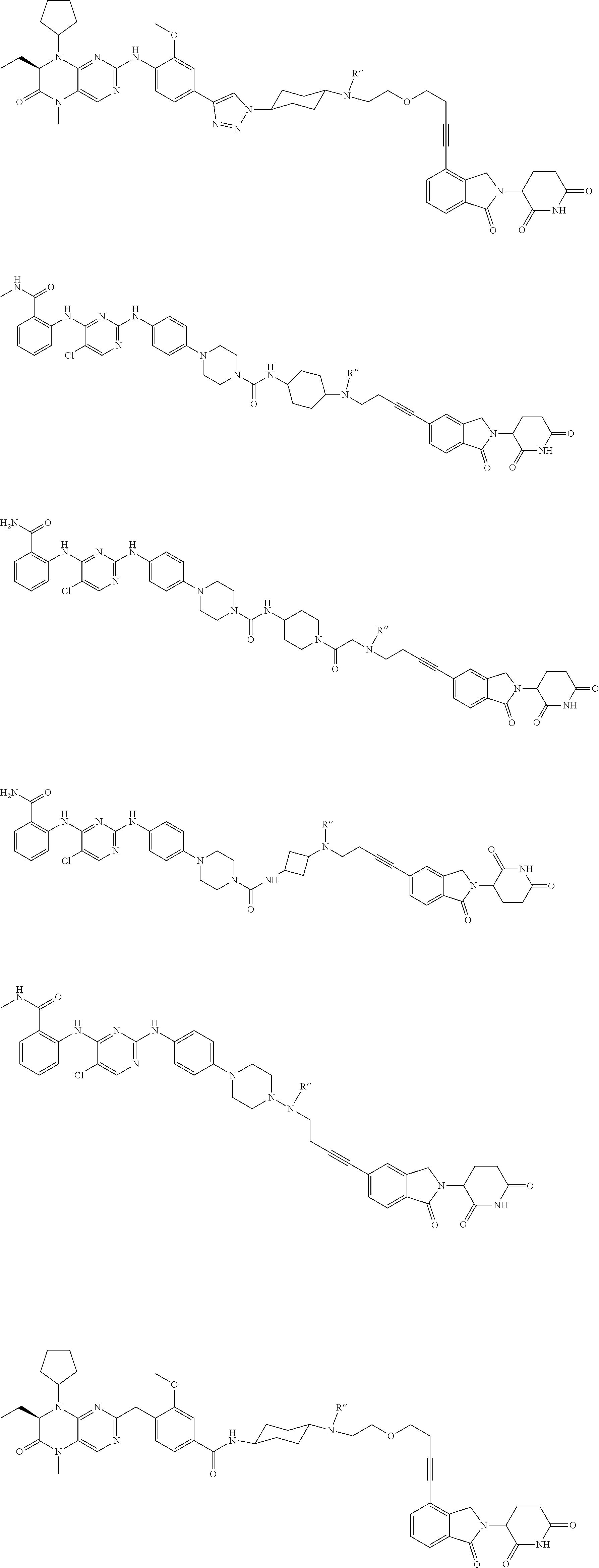

In another preferred embodiment, Ab is connected with W⁴-L3-W³— of formula III (preferably,

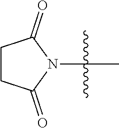 [SMILES: *N1C(=O)CCC1=O]

or —NH₂ group in W⁴-L3-W³—), through amino acid at N-terminal or C-terminal, or a side chain of amino acid (preferably, an amino acid side chain selected from the group consisting of Lys, and Cys), or a sulfhydryl group formed by reducing and opening disulfide bonds.

In another preferred embodiment, the target molecule is target molecule A or target molecule T.

In another preferred embodiment, the target molecule A or T include small molecules, nanocarriers, or combinations thereof.

In another preferred embodiment, the target molecule A and T are each independently target molecule selected from the group consisting of or target molecules targeting targets (such as respective enzymes or receptors) selected from the group consisting of: folic acid, HSP90, TINFRm, TNFR2, NADPH oxidase, BclIBax, C5a receptor, HMG-CoA reductase, PDE I-V, Squalene cyclase inhibitors, CXCR1, CXCR2, Nitric oxide (NO)synthase, cyclo-oxygenase 1-2, 5HT receptors, dopamine receptors, G-proteins, Gq, Histamine receptors, Lipoxygenases, Tryptase serine protease, Thymidylate synthase, Purine nucleotide phosphorylase, GAPDH trypanosomal, Glycogen phosphorylase, Carbonic anhydrase, Chemokine receptors, JAW STAT, RXR and its analogues, HIV 1 protease, HIV 1 integrase, Influenza, hepatitis B reverse transcriptase, neuraminidase, Sodium channel, MDR, protein P1-glycoprotein, Tyrosine kinases, CD23, CD124, TK p56 lck, CD4, CD5, IL-1 receptor, IL-2 receptor, TNF-αR, ICAM1, Ca+ channels, VCAM, VLA-4 integrin, VLA-4 integrin, Selectins, CD40/40L, Newokinins and receptors, Inosine monophosphate dehydrogenase, p38 p38 MAP kinase, Interleukin-1 converting enzyme, Caspase, HCV NS3 protease, HCV—NS3 RNA helicase, Glycinamide ribonucleotide formyl transferase, rhinovirus 3C protease, HSV-I, CMV, ADP-polymerae, CDK, VEGF, oxytoxin receptor, msomal transfer protein inhibitor, Bile acid transfer protein inhibitor, 5-a reductase, Angiotensin 11, Glycine receptors, noradrenaline reuptake receptor, Endothelin receptors, Neuropeptide Y and receptors, Estrogen receptors, AMP, AMP deaminase, ACC, EGFR, Farnesyltransferase.

In another preferred embodiment, the polypeptide element includes antibody, protein, or combinations thereof.

In another preferred embodiment, the antibody comprises nanobody, small molecule antibody (minibody), or combinations thereof.

In another preferred example, the polypeptide element is antibody; preferably, the antibody comprises nanobody, small molecule antibody (minibody), antibody fragment (such as scFv, Fab), double antibody (Dibody) and the like.

In another preferred embodiment, the targets of the polypeptide (targeting polypeptide) include but are not limited to: EGFR, FGFR, SSTR1-14, GnRH, TRPV1-6, RGD, iRGD and the like.

In another preferred example, the antibody can bind to an antigen or receptor selected from the group consisting of (for example, bind to one (i.e., monofunctional antibody) or two (i.e., bifunctional antibody) or more (i.e., multifunctional antibody) antigens and/or receptors selected from the group consisting of) DLL3, EDAR, CLL1, BMPR1B, E16, STEAP1, 0772P, MPF, 5T4, NaPi2b, Sema 5b, PSCA hlg, ETBR, MSG783, STEAP2, TrpM4, CRIPTO, CD21, CD22, CD79b, CD19, CD37, CD38, CD138, FcRH2, B7-H4, HER2, NCA, MDP, IL20Rα, Brevican, EphB2R, ASLG659, PSCA, GEDA, BAFF-R, CD79a, CXCR5, HLA-DOB, P2X5, CD72, LY64, FcRH1, IRTA2, TENB2, PMEL17, TMEFF1, GDNF-Ra1, Ly6E, TMEM46, Ly6G6D, LGR5, RET, LY6K, GPR19, GPR54, ASPHD1, Tyrosinase, TMEM118, GPR172A, MUC1, CD70, CD71, MUC16, methothelin, FOLR1, TroP1-2, gpNMB, EGFR, ENPP3, PSMA, CA6, GPC-3, PTK7, CD44, CD56, TIM-1, Cadherin-6, ASG-15ME, ASG-22ME, CanAg, AXL, CEACAM5, EphA4, cMet, FGFR2, FGFR3, CD123, Her3, LAMP1, LRRC15, TDGF1, CD66, CD25, BCMA, GCC, Noch3, cMet, EGFR and CD33, or receptors such as CD70, Trop2, PD-L1, CD47, CLDN-18.2. In another preferred embodiment, the target molecule of the present invention can also bind to receptors that can be targeted by specific small molecules, such as folic acid, HSP90, glucose transporter 1 (GLUT1), aminopeptidase N (APN), low-density lipoprotein receptor-related protein 1 (LRP1), prostate-specific membrane antigen (PSMA), integrin αvβ3, bombesin receptor, somatostatin receptor (SSTR), tumor hypoxic microenvironment, and carbonic anhydrase IX (CAIX) and other receptors.

In another preferred embodiment, R_{T} is selected from groups shown in Table B1 and B2.

In another preferred embodiment, the moiety of E3 ligase ligand A1 is selected from: the A¹ groups in WO2017/176957 A1 (preferably, the corresponding moieties of A-10, A-11, A-15, A-28, A-48, A-69, A-85, A-93, A-98, A-99 or A-101 in WO2017/176957 A1).

In another preferred embodiment, the moiety of E3 ligase ligand is selected from:

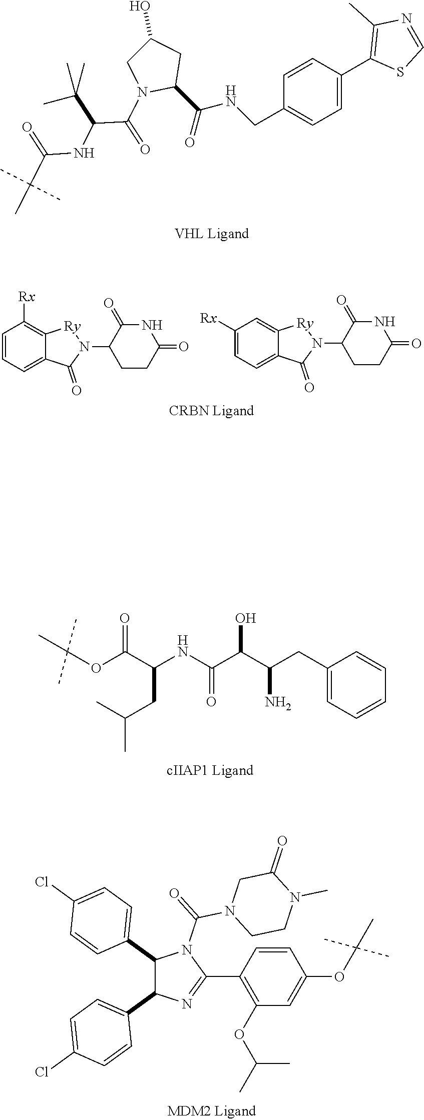

In each formula, the dotted line indicates the position of connection with other parts (i.e., the position of connection with R_{T}-L1);
wherein Rx is each independently selected from the group consisting of null, NH, NH—CO, O, S, SO, SO₂, SO₂(NH₂)NH, C1˜C4 alkylene, C2˜C5 alkenylene, C2˜C5 alkynylene; R_{y} is C═O, C═S or CH₂.

In another preferred embodiment, the moiety of E3 ligase ligand is selected from the groups shown in Table C.

In another preferred embodiment, when R_{E3} is

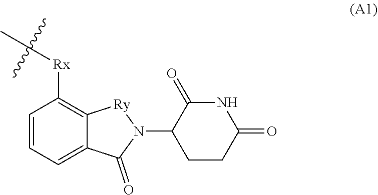

(preferably, A1.2 in Table B), the conjugate of formula I is of formula 1-1, R_{T}—W¹-L5-W^{b}—C≡C—R_{E3} (1-1); preferably, at least one of M in L5 is O and/or W¹ is NH or NH—Cr², and/or W^{b} is CH²; more preferably, in L5, 7≤o1+o2≤12.

In another preferred embodiment, when R_{E3} is

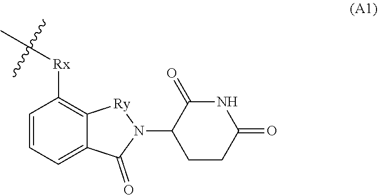

(preferably, A1.2 in Table B), the conjugate of formula I is of R_{T}—W^{a}—Cr¹—Cr²-(M)ₒ₃-W²—R_{E3}, and neither of Cr¹ and Cr² is null; preferably, L2 is -(M)ₒ₃-, and subscript o3 is 1, 2, 3, 4, or 5.

In another preferred embodiment, R_{T}, L1, R_{E3}, W¹, L2, W², W, subscript s, R, R^{a}, R^{b}, M^{L}, subscript o, M, M^{T}, M^{N}, R′, R″, W³, L^{T1}, W^{P1}, R_{P}, subscript q1, W⁴, R_{P1}, W″, W^{P2}, subscript s1, subscript s2, R′″, W⁵, L^{T2}, W⁶, L^{T3}, R_{P2}, M′, W^{Y}, subscript t1, subscript t2, subscript t3, subscript t4, W_{X}, W′, subscript s3, subscript s4, W⁶, subscript s6, R^{c}, L5, L6, subscript o1, subscript o2, L7, Ar1, Cr¹, Cr², W^{a}, W^{b}, R_{X}, R_{Y}, subscript n, R^{Pa}, R₂₀, R₂₁, subscript q, subscript p, and M^{a} are each independently the corresponding groups in sub-formula or specific compounds described herein (such as sub-formulas in Group 1, Group 1a, Group 2, Group 2a, Group 3, Group 3a and the like, and the specific compounds described in the Preparation Examples).

In another preferred embodiment, the conjugate is TED compound described in the sixth aspect.

In another preferred embodiment, the conjugate is ACTED compound described in the seventh aspect.

In another preferred embodiment, the conjugate is not the specific compound disclosed in PCT/CN2019/110225 and PCT/IB2021/052954.

In another preferred embodiment, the conjugate is not the specific compound described in Table D in PCT/CN2019/110225, the specific compounds described in Table D is as follows:

| | |
|---|---|
| 838 | 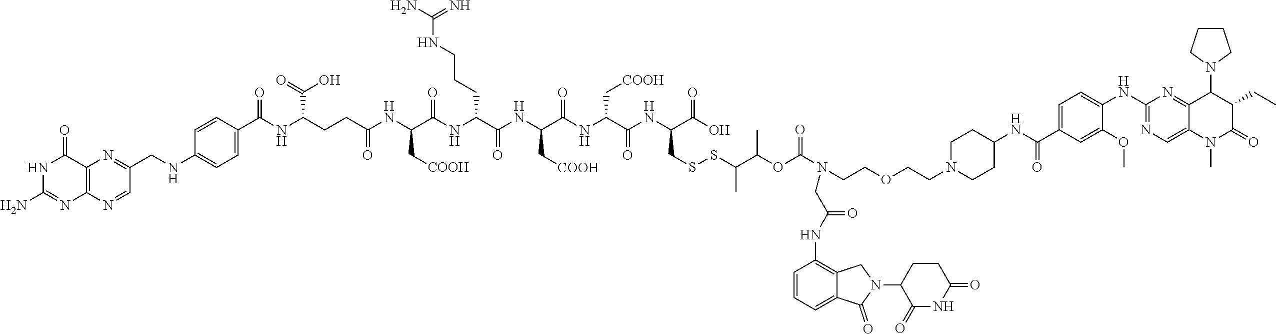 [SMILES: CC[C@@H]1C(=O)N(C)c2cnc(Nc3ccc(C(=O)NC4CCN(CCOCCN(CC(=O)Nc5cccc6c5CN(C5CCC(=O)NC5=O)C6=O)C(=O)OC(C)C(C)SSC[C@@H](NC(=O)[C@@H](CC(=O)O)NC(=O)[C@@H](CC(=O)O)NC(=O)[C@@H](CCCNC(=N)N)NC(=O)[C@@H](CC(=O)O)NC(=O)CC[C@H](NC(=O)c5ccc(NCc6cnc7nc(N)[nH]c(=O)c7n6)cc5)C(=O)O)C(=O)O)CC4)cc3OC)nc2C1N1CCCC1] |
| 839 | 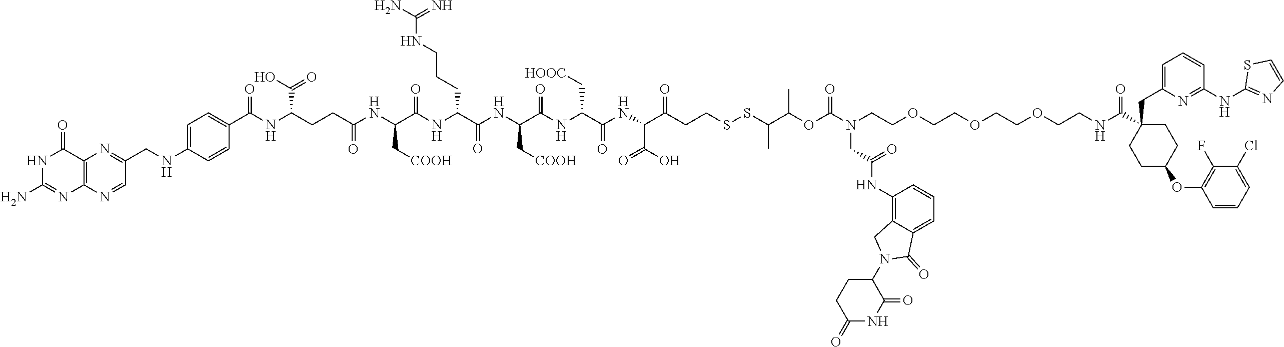 |
| 829 | 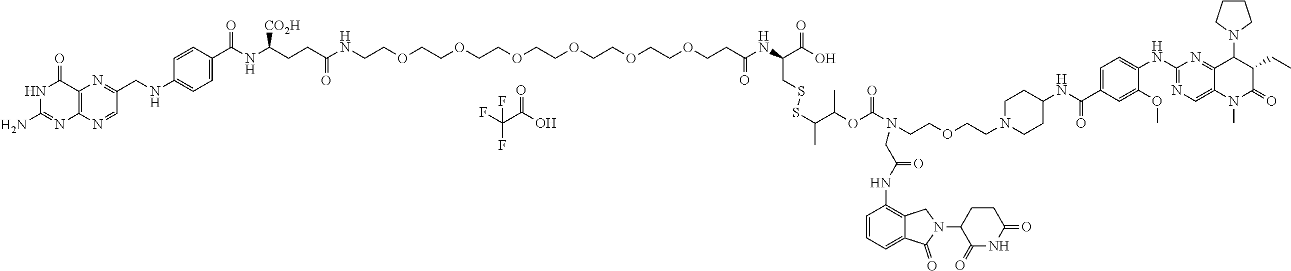 [SMILES: CC[C@@H]1C(=O)N(C)c2cnc(Nc3ccc(C(=O)NC4CCN(CCOCCN(CC(=O)Nc5cccc6c5CN(C5CCC(=O)NC5=O)C6=O)C(=O)OC(C)C(C)SSC[C@@H](NC(=O)CCOCCOCCOCCOCCOCCOCCNC(=O)CC[C@@H](NC(=O)c5ccc(NCc6cnc7nc(N)[nH]c(=O)c7n6)cc5)C(=O)O)C(=O)O)CC4)cc3OC)nc2C1N1CCCC1 ; O=C(O)C(F)(F)F] |

In another preferred embodiment, the conjugate is not the following compounds described in PCT/IB2021/052954:

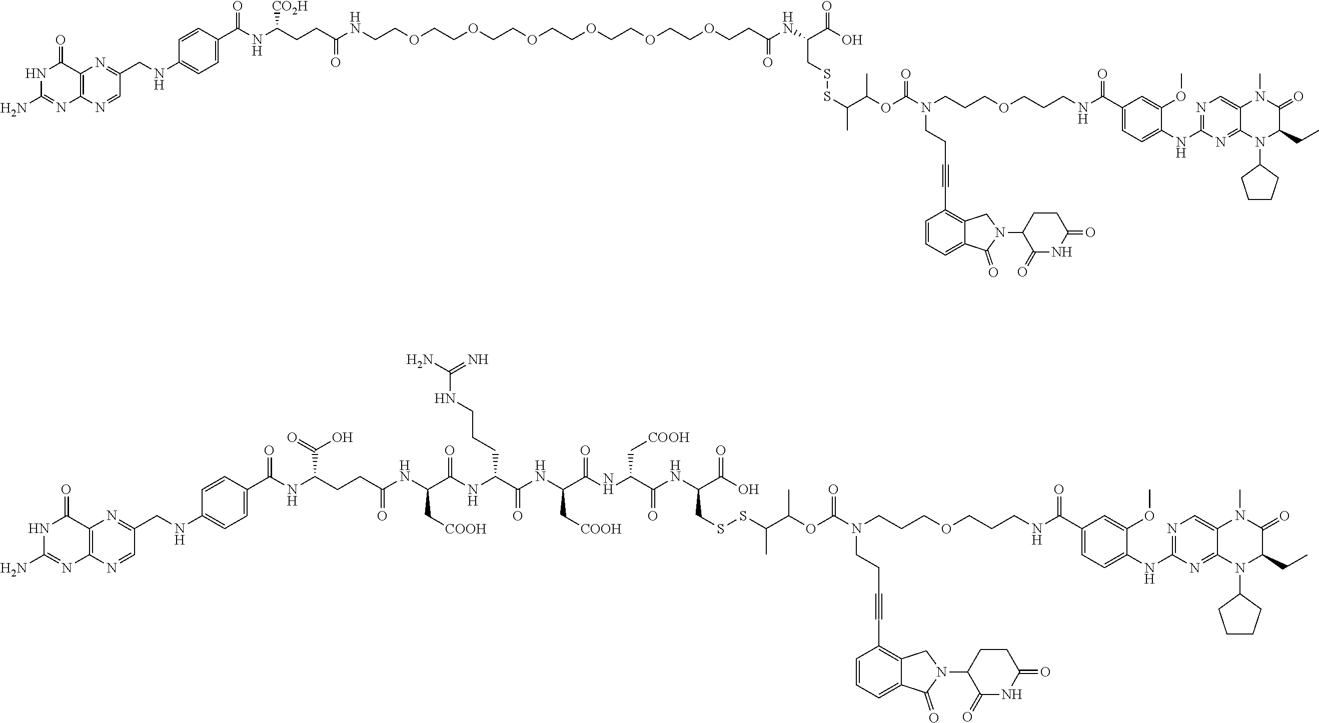

compounds 1216, 1229, 1231, 1233 in Table D of PCT/IB2021/052954.

In the second aspect of the present invention, provided is a pharmaceutical composition, wherein the pharmaceutical composition includes the conjugate described in the first aspect and pharmaceutically acceptable carriers.

In the third aspect of the present invention, provided is a use of the conjugate according to the first aspect in the preparation of a drug for the treatment or prevention of diseases associated with excessive target protein.

In the fourth aspect of the present invention, provided is a use of the conjugate according to the first aspect in the treatment or prevention of diseases associated with excessive target protein.

In the fifth aspect of the present invention, provided is a method for reducing the content of target proteins in the cell, comprising contacting the cell with the conjugate as described in the first aspect, thereby reducing the content of target proteins in the cell.

In another preferred embodiment, the method is a method in vitro.

In another preferred embodiment, the method is non-diagnostic and non-therapeutic.

In the sixth aspect of the present invention, provided is a TED compound or a pharmaceutically acceptable salt thereof, wherein the TED compound is of formula VI;

R_{T}W¹-(M^{L})ₒ-W²—R_{E3} (VI)
wherein
M^{L} is each independently M or M^{N}
M, M^{N}, R_{E3}, R_{T}, W¹, W² and subscript o are as defined in formula I.

In another preferred embodiment, the TED compound is of formula IV.

In another preferred embodiment, the TED compound is of formula 1a-1, 1a-2, 1a-3, 2a or 3a.

In another preferred embodiment, the TED compound is used for coupling with R_{P}.

In another preferred embodiment, the TED compound is coupled with R_{P} through —W³-L3-W⁴—.

In another preferred embodiment, the TED compound is a compound selected from Group 1, Group 2 and Group 3, and R and R¹ are each independently R′.

In another preferred embodiment, the TED compound is selected from Table A1 and A2.

In the seventh aspect of the present invention, provided is an ACTED compound, or a pharmaceutically acceptable salt thereof, wherein the ACTED compound is of formula VII;

R_{T}W¹-(M^{L})ₒ-W²—R_{E3} (VII)
wherein
M^{L} is each independently M or M^{T}
M, M^{T}, R_{E3}, R_{T}, W¹, W² and subscript o are as defined in formula I.

In another preferred embodiment, the ACTED compound is of formula V.

In another preferred embodiment, the ACTED compound is of formula X.

In another preferred embodiment, the ACTED compound is of formula 1b-1, 1b-2, 1b-3, 2b or 3b.

In another preferred embodiment, the ACTED compound is a compound selected from Group 1, Group 2 and Group 3, and R and R¹ are each independently R″.

In another preferred embodiment, the ACTED compound is selected from: Table D.

It should be understood that within the scope of the present invention, the above technical features of the present invention and the technical features specifically described in the following (eg, examples) can be combined with each other, thereby forming a new or preferred technical solution. Due to space limitations, it will not be repeated herein.

### DESCRIPTION OF THE DRAWINGS

FIG. 1 shows the degradation of Aurora A in NCI-H821 cell line by the compounds of the present invention.

FIG. 2 shows the degradation of BRD4 and PLK1 in MV4;11 cell line by the compounds of the present invention.

FIG. 3 is a graph showing the tumor inhibitory effect of the compound (UB-181322) of the present invention when administered by injection at 23 mg/kg once every two days. Compared with the blank group, UB-181322 shows the effect of inhibiting tumor growth (A), and the mouse body weight does not change significantly during the administration period, and the toxicity is low (B).

### EMBODIMENTS FOR CARRYING OUT THE INVENTION

After extensive and deep research, the inventors developed a TED conjugate with novel structures for the first time, the TED conjugates of the present invention have a structure of formula I. In addition, the TED conjugates of the present invention are very suitable for further connecting with polypeptide elements (especially antibodies, protein ligands) and/or other molecules with targeting properties, or after further connecting with polypeptide elements and/or other molecules with targeting properties and the like, or polypeptide elements and/or other molecules with targeting properties in the conjugates further connected with polypeptide elements and/or other molecules with targeting properties, the conjugates of the present invention, thereby possesses excellent targeting properties (for example specifically targeting tumor cells), improve drug selectivity, implements more precise degradation of pathogenic proteins, reduces the possible systemic toxicity induced by non-specific degradation, and is possible to overcome the difficulties encountered in drug absorption and metabolism, and eliminates the possibility for producing drug resistance. The inventor has completed the present invention on this basis.

### Terms

As used herein, the terms “compound of the present invention”, and “conjugate of the present invention” are used interchangeably and refer to the compound or the conjugate of formula I described in the first aspect of the present invention.

As used herein, unless otherwise stated, the term “alkyl”, by itself or as a part of another substituent means a straight or branched chain hydrocarbon radical with designated carbon atoms (i.e. C₁₋₆ means 1-6 carbons). Preferably, alkyl contains 1 to 4 carbons, i.e. C₁₋₄ alkyl. Examples of alkyl include, but are not limited to, methyl, ethyl, n-propyl, isopropyl, n-butyl, 1-butyl, iso-butyl, sec-butyl, n-pentyl, n-hexyl, n-heptyl, n-octyl, and the like. The term “alkenyl” refers to an unsaturated alkyl having one or more double bonds. Preferably, alkenyl contains 2 to 4 carbons, i.e. C₂₋₄ alkenyl. Similarly, the term “alkynyl” refers to an unsaturated alkyl having one or more triple bonds. Preferably, alkynyl contains 2 to 4 carbons, i.e. C₂₋₄ alkynyl. Examples of such unsaturated alkyl include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “cycloalkyl” refers to hydrocarbon rings having an indicated number of ring atoms (e.g., C₃₋₆ cycloalkyl) and being fully saturated or having no more than one double bond between ring vertices.

As used herein, the term “cycloalkyl” refers to hydrocarbon rings having an indicated number of ring atoms (e.g., C₃₋₈ cycloalkyl) and being fully saturated or having no more than one double bond between ring vertices. This term is also meant to contain bicyclic and polycyclic hydrocarbon rings such as bicyclo[2.2.1]heptane, bicyclo[2.2.2]octane, etc. The term “heterocycloalkyl” refers to a cycloalkyl that contains one to five heteroatoms selected from N, O, and S, wherein the nitrogen and sulfur atoms are optionally oxidized, and the nitrogen atom(s) are optionally quaternized. The heterocycloalkyl may be a monocyclic, a bicyclic or a polycylic ring system. Non limiting examples of heterocycloalkyl include pyrrolidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrhydrothiophene, quinuclidine, and the like. The heterocycloalkyl can be attached to the rest of the molecule via a ring carbon or a heteroatom. For terms such as cycloalkylalkyl and heterocycloalkylalkyl, it is meant that a cycloalkyl or a heterocycloalkyl is attached through an alkyl or alkylene linker to the rest of the molecule. For example, cyclobutylmethyl—is a cyclobutyl ring that is attached to a methylene linker of the rest of the molecule.

The term “alkylene” by itself or as a part of another substituent means a divalent radical derived from an alkane, as exemplified by —CH₂CH₂CH₂CH₂—. Typically, an alkyl (or alkylene) will have from 1 to 24 carbon atoms, with those groups having 10 or fewer carbon atoms being preferred in the present disclosure. A “lower alkyl” or “lower alkylene” refers to an alkyl or alkylene with shorter chain, generally having four or fewer carbon atoms. Similarly, “alkenylene” and “alkynylene” refer to the unsaturated forms of “alkylene” having a double or triple bond, respectively.

Unless otherwise specified, the term “heteroalkyl” by itself or in combination with other terms refers to a stable linear or branched or cyclic hydrocarbon group or a combination thereof, consisting of a specified number of carbon atoms and 1 to 3 heteroatoms selected from O, N, Si and S, and wherein nitrogen and sulfur atoms are optionally oxidized, and nitrogen heteroatoms can be optionally quaternized. The heteroatoms O, N and S can be placed at any internal position of the heteroalkyl. The heteroatom Si may be placed at any position of the heteroalkyl, including the position at which the alkyl is attached to the rest of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as —CH₂—NH—OCH₃ and —CH₂—O—Si(CH₃)₃. Similarly, unless otherwise specified, terms “heteroalkenyl” and “heteroalkynyl” by themselves or in combination with another term refer to alkenyl or alkynyl, respectively, that contain a specified number of carbons and 1 to 3 heteroatoms selected from O, N, Si and S, and wherein the nitrogen and sulfur atoms may optionally be oxidized and the nitrogen heteroatom may optionally be quaternized. The heteroatoms O, N and S can be placed at any internal position of the heteroalkyl.

The term “heteroalkylene” by itself or as a part of another substituent means a saturated or unsaturated or polyunsaturated divalent radical, derived from heteroalkyl, as exemplified by —CH₂—CH₂—S—CH₂CH₂— and —CH₂—S—CH₂—CH₂—NH—CH₂—, —O—CH₂—CH═CH—, —CH₂—CH═C(H)CH₂—O—CH₂— and —S—CH₂—C≡C—. For heteroalkylene, heteroatoms can also occupy either or both of the chain terminals (e.g., alkyleneoxy, alkylenedioxy, alkyleneamino, alkylenediamino, and the like).

The terms “alkoxy”, “alkylamino” and “alkylthio” (or thioalkoxy) are used in their conventional meanings, refer to the alkyl attached to the rest of the molecule via oxygen atom, amino, or sulfur atom, respectively. Additionally, for dialkylamino, the alkyl portions can be the same or different and can also be combined to form a 3-7 membered ring with the nitrogen atom to which each alkyl is attached. Accordingly, a group represented as —NR^{a}R^{b} is meant to include piperidinyl, pyrrolidinyl, morpholinyl, azetidinyl and the like.

Unless otherwise stated, the term “halo” or “halogen” by themselves or as a part of another substituent, means, fluorine, chlorine, bromine, or iodine atom. Additionally, terms such as “haloalkyl,” are meant to include monohaloalkyl or polyhaloalkyl. For example, the term “C₁₋₄ haloalkyl” is mean to include trifluoromethyl, 2,2,2-trifluoroethyl, 4-chlorobutyl, 3-bromopropyl, and the like.

Unless otherwise stated, the term “aryl” means, a polyunsaturated, typically aromatic, hydrocarbon group which can be a single ring or multiple rings (at most three rings) which are fused together or linked covalently. The term “heteroaryl” refers to aryl (or ring) that contains one to five heteroatoms selected from N, O, and S, wherein the nitrogen and sulfur atoms are optionally oxidized, and the nitrogen atom(s) are optionally quaternized. A heteroaryl can be attached to the rest of the molecule through heteroatom. Non-limiting examples of aryl include phenyl, naphthyl and biphenyl, while non-limiting examples of heteroaryl include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridinyl, benzothiazolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. Substituents for the above-stated aryl and heteroaryl ring systems are selected from the group of acceptable substituents described below.

For brevity, the term “aryl” when used in combination with other terms (e.g., aryloxy, arylthio, arylalkyl) includes both aryl and heteroaryl rings as defined above. Thus, the term “arylalkyl” is meant to include those radicals in which an aryl is attached to an alkyl that is attached to the rest of the molecule (e.g., benzyl, phenethyl, pyridylmethyl and the like).

The above terms (e.g., “alkyl,” “aryl” and “heteroaryl”), in some embodiments, include both substituted and unsubstituted forms of indicated radicals. The preferred substituents for each type of group are provided below. For brevity, the terms aryl and heteroaryl refer to the substituted or unsubstituted forms as provided below, while the term “alkyl” and related aliphatic radicals are meant to refer to the unsubstituted form, unless indicated to be substituted.

Substituents for the alkyl (including those groups often referred to as alkylene, alkenyl, alkynyl and cycloalkyl) can be a variety of groups selected from the group consisting of: -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —CN and —NO₂, in a number ranging from zero to (2M′+1), wherein M′ is the total number of carbon atoms in such radical. R′, R″ and R′″ are each independently refer to hydrogen, unsubstituted C₁₋₈ alkyl, unsubstituted heteroalkyl, unsubstituted aryl, aryl substituted with 1-3 halogens, unsubstituted C₁₋₈ alkyl, C₁₋₈ alkoxy or C₁₋₈ thioalkoxy, or unsubstituted aryl-C₁₋₄ alkyl. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. The term “acyl” as used by itself or as a part of another group refers to groups wherein two H on the carbon that is closest to the point of attachment for the radical is replaced with the substituent ═O (e.g., C(O)CH₃, —C(O)CH₂CH₂OR′ and the like).

Similarly, substituents for aryl and heteroaryl are varied and generally selected from -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R″′, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —N₃, perfluoro(C₁-C₄)alkoxy, and perfluoro(C₁-C₄)alkyl, in a number ranging from 0 to a total number of open valences on the aromatic ring system; and wherein R′, R″ and R′″ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl, and unsubstituted aryloxy-C₁₋₄ alkyl. Other suitable substituents include each of the above aryl substituents attached to a ring atom by an alkylene containing 1-4 carbon atoms.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer selected from 0 to 2. Alternatively, two of the substituents on adjacent atoms of the aryl or heteroaryl ring may optionally be replaced with formula -A-(CH₂)ᵣ—B—, wherein A and B are independently —CH₂—, —O—, —NH—, —S—, —S(O)—, —S(O)₂—, —S(O)₂NR′— or a single bond, and r is an integer selected from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, wherein s and t are independently integers selected from 0 to 3, and X is —O—, —NR′—, —S—, —S(O)—, —S(O)₂— or —S(O)₂NR′—. The substituent R′ in —NR′— and —S(O)₂NR′— is selected from hydrogen or unsubstituted C₁₋₆ alkyl.

In the present invention, when cycloalkyl or heterocycloalkyl is a divalent group, the cycloalkyl or heterocycloalkyl may lose two hydrogens on the same ring atom (on ring carbon atom) thereby connecting with other chain atoms on the chain (forming a structure similar to a spirocyclic ring), or may lose two hydrogens on different ring atoms thereby connecting with other chain atoms on the chain (such as -cyclopentylidene-).

As used herein, the term “heteroatom” is meant to include oxygen (O), nitrogen (N), sulfur (S) and silicon (Si).

As used herein, the term “protecting group” refers to a group that is used to protect the active group from participating in a reaction and is easy to remove; similarly, the term “amino protecting group” refers to a group that is used to protect the active amino group from participating in a reaction and is easy to remove. Examples of amino protecting group include but are not limited to: —COO—C₁₋₆ alkyl (such as tert-butoxycarbonyl (Boc)), COO-aryl or heteroaryl (such as —COO-phenyl), —COO—C1-2alkylene-aryl or heteroaryl (such as benzyloxycarbonyl (CBz)); the amino protecting group can also be a group formed by the reaction of organic or inorganic acids with H in the amino group (such as (n-)phosphoryl (—H₂PO₃), etc.).

For the compounds provided herein, a bond that is drawn from a substituent (typically an R group) to the center of an aromatic ring (e.g., benzene, pyridine, and the like) will be understood to refer to a bond providing a connection at any of the available vertices of the aromatic ring. In some embodiments, the depiction will also include connection at a ring which is fused to the aromatic ring. For example, a bond drawn to the center of the benzene portion of an indole, will indicate a bond to any available vertex of the six- or five-membered ring portions of the indole.

As used herein, term “amino acid residue” refers to a group formed by the removal of an H from —NH₂ at the N-terminal and the removal of —OH from —COOH at the C-terminal of an amino acid. Unless otherwise defined, as used herein, amino acids include natural or non-natural amino acids, including D and/or L-type amino acids. Examples of amino acids include, but are not limited to, Ala (A), Arg (R), Asn (N), Asp (D), Cys (C), Gln (Q), Glu (E), Gly (G), His (H), Ile (I), Leu (L), Lys (K), Met (M), Phe (F), Pro (P), Ser (S), Thr (T), Trp (W), Tyr (Y), Val (V). Preferably, the amino acid, as used herein, is an amino acid selected from the group consisting of: L-glycine (L-Gly), L-alanine (L-Ala), β-alanine (β-Ala), L-glutamic acid (L-Glu), L-aspartic acid (L-Asp), L-histidine (L-His), L-Arginine (L-Arg), L-Lysine (L-Lys), L-Valine (L-Val), L-Serine (L-Ser), and L-Threonine (L-Thr). In addition, when amino acids have 2 or more amino groups and/or 2 or more carboxyl groups, the term also includes groups formed by the removal of H from —NH₂ and the removal of —OH from —COOH on different carbon atoms, such as the divalent group —C(O)—(CH₂)₂—C(COOH)—NH— formed by the removal of H from —NH₂ and the removal of H from —COOH on non-α-position in a glutamic acid, respectively.

The term “pharmaceutically acceptable salts” is meant to include salts formed of the active compounds with relatively nontoxic acids or bases, depending on the particular substituents found on the compounds described herein. When compounds of the present disclosure contain relatively acidic functional groups, base addition salts can be obtained by contacting the neutral form of such compounds with a sufficient amount of the desired base, either neat or in a suitable inert solvent. Examples of salts derived from pharmaceutically acceptable inorganic bases include aluminum, ammonium, calcium, copper, iron, ferrous, lithium, magnesium, manganese, manganous, potassium, sodium, zinc, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functional groups, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts formed of amino acids such as arginate and the like, and salts formed of organic acids like glucuronic or galactunoric acids and the like. Certain specific compounds of the present disclosure contain both basic and acidic functional groups that allow the compounds to be converted into either base or acid addition salts.

The neutral forms of the compounds may be regenerated by contacting the salt with a base or acid and isolating the parent compound in a conventional manner. The parent form of the compound differs from the various salt forms thereof in certain physical properties, such as solubility in polar solvents, but in addition to the above, those salts are equivalent to the parent form of the compound for the purposes of the present invention.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, when placed in a transdermal patch reservoir containing suitable enzymes or chemical reagents, the prodrug can be slowly converted to the compound of the invention.

Certain compounds of the present disclosure can exist in unsolvated forms as well as solvated forms, including hydrated forms. The solvated forms are generally equivalent to the non-solvated forms and should be included in the scope of the present invention. Certain compounds of the present disclosure may exist in polycrystallie or amorphous forms. Generally, as for the application considered in the present invention, all physical forms are equivalent and should be included in the scope of the present invention.

Certain compounds of the present disclosure possess asymmetric carbon atoms (optical centers) or double bond; the racemates, diastereomers, geometric isomers, regioisomers and individual isomers (e.g., separate enantiomers) are all intended to be encompassed within the scope of the present disclosure. When compounds are provided herein with an identified stereochemistry (indicated as R or S, or with dashed or wedge bond designations), those compounds will be understood by those skilled in the art to be substantially free of other isomers (e.g., at least 80%, 90%, 95%, 98%, 99%, and up to 100% free of the other isomer).

The compounds of the present disclosure may also contain unnatural proportions of atomic isotopes at one or more of isotopic atoms that constitute such compounds. The unnatural proportions of certain isotope can be defined as the amount from the naturally found amount of the atom discussed to 100% of that atom. For example, the compounds may incorporate radioactive isotopes, such as tritium (³H), iodine-125 (¹²⁵I) or carbon-14 (¹⁴C), or non-radioactive isotopes, such as deuterium (²H) or carbon-13 (¹³C). Such isotopic variants may provide additional uses in addition to those described in this application. For instance, isotopic variants of the compounds of the disclosure may find additional utility, including but not limited to, as diagnostic and/or imaging reagents, or as cytotoxic/radiotoxic therapeutic agents. Additionally, isotopic variants of the compounds of the disclosure can have altered pharmacokinetic and pharmacodynamic characteristics which can contribute to enhanced safety, tolerability or efficacy during treatment. All isotopic variations of the compounds of the present disclosure, whether radioactive or not, should be encompassed within the scope of the present disclosure.

### Targeted Enzyme Degradation (TED) Platform

The present invention provides a Targeted Enzyme Degradation (TED) platform on basis of the conjugate of the present invention, which utilizes the “intracellular cleaner”—ubiquitin proteasome system.

Typically, according to the TED technology of the present invention, which can utilize cell's intrinsic protein destruction mechanism to remove specific oncogenic and pathogenic proteins from the cell, therefore it is an alternative method of targeted therapy.

Different from the action mechanism of conventional protein inhibitors, TED technology of the present invention relates to a bifunctional hybrid compound, one side of which is used to bind target proteins, and the other side is used to bind E3 ligases, enabling the target proteins binding the E3 ligases, and the target proteins being ubiquitinated, thereby being degraded by the proteome. Theoretically, TED technology only provides binding activity without directly inhibiting the functional activity of the target protein, and can be reused. Therefore, TED technology has excellent application prospects.

In particular, the optimized TED molecules of the present invention have superior target protein degradation ability on target protein, thereby inhibiting the growth of lesional cells. In addition, the TED (i.e., R_{TED}) of the present invention is coupled with a ligand targeting tumor tissue (such as folic acid etc.) through a linker with a specific structure, (for example, there is a moiety of bivalent linker that is cleavable at the cell surface or within the cytoplasm (e.g., —S—S— or peptide chain such as -AN-, -AAN-, -VA-, -GGFG-, -AAFG, -VCit-, -VL-) or a moiety of hydrophilic bivalent linker (e.g., PEG chain, side chain containing acidic functional groups such as —SO₃H, —PO₃H₂, —COOH, etc.) on the linker chain) to form the ACTED molecule (or conjugate) described herein.

When ACTED with the above structure enters the blood circulation, it can bind to antigens or receptors on the surface of the tumor via ligand moiety coupled through linker with specific structure, thus rapidly enriched into tumor tissue. After binding with the tumor, the ACTED of the present invention may play the following roles: for example, 1. entering cells through receptor-mediated endocytosis, being cleaved under acidic environment, by GSH (glutathione), or specific enzymes, releasing active molecules TED, then TED binding with intracellular target protein and E3 enzyme, degrading target protein via ubiquitin-mediated proteasome, then killing tumor cells; 2. Cleaving ACTED under acidic environment of microenvironment, by GSH, or specific enzymes on the cell surface, releasing TED, then TED diffusing into cells to play the role of degrading target proteins and killing tumor cells. It can be seen that the present invention also provide a Pro-drug conjugate based on the microenvironment and hypoxic condition of targeted tumors.

Therefore, the advantages of the ACTED of the present invention can be divided into two aspects: 1. ACTED enriched more TED into tumor tissue and facilitated TED to enter into tumor cells, degrading target proteins thereby killing tumor cells, thus improving the utilization rate of TED; 2. ACTED rarely binds to normal cells, thus less TED enters normal tissues during circulation, thererby reducing toxic side effects.

Some exemplary linkers containing ligands targeting tumor tissue are as follows, wherein only representative linker fragments are exemplarily listed. It should be understood that other common linker groups, such as —NHCO—, —NH—, —CO—, methylene, common amino acid residues, may also be present between each fragment
(1) for example, the linker can covalently bind to —SH on cysteine in Ligand:

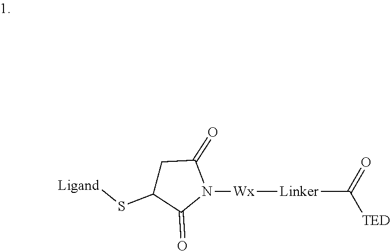

In this formula,

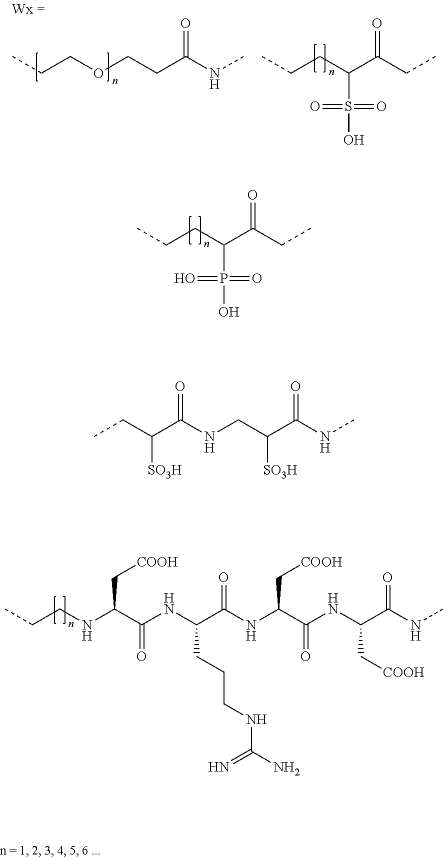

wherein Wx can be the above fragments being used alone or used in combination

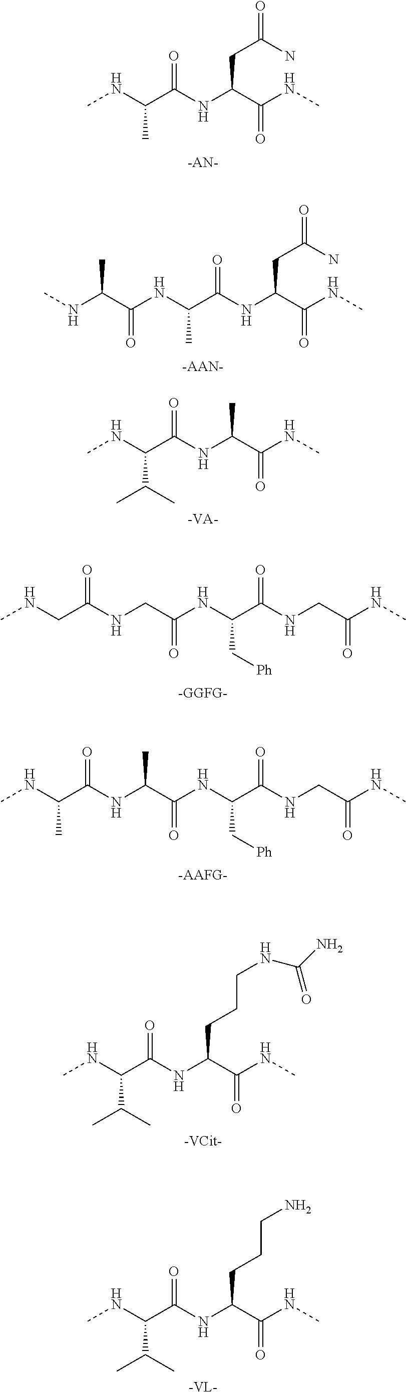

(2) for example, the linker covalently bind to —NH₂ on lysine in Ligand:

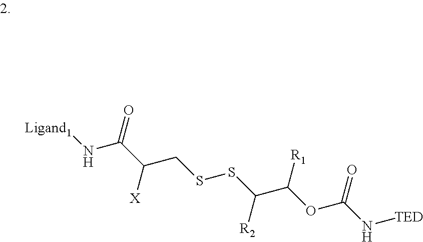

Ligand1=peptide, FA, HSP90 binder . . .
R1, R2═H, -Me
X═H, NH2

Some exemplary structures of TED coupled with double ligand are as follows

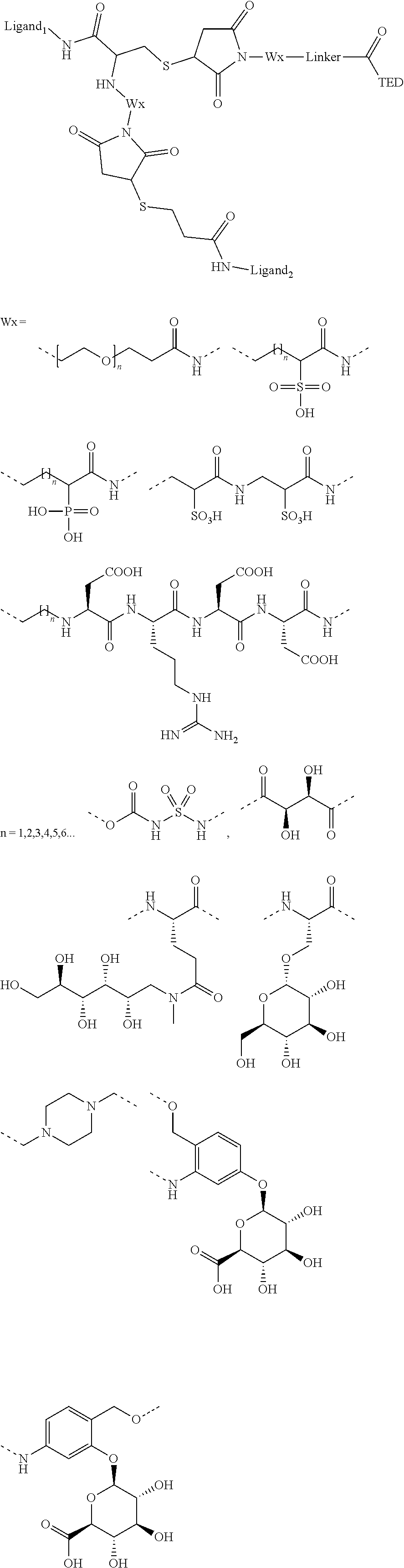

wherein Wx can be the above fragments being used alone or used in combination

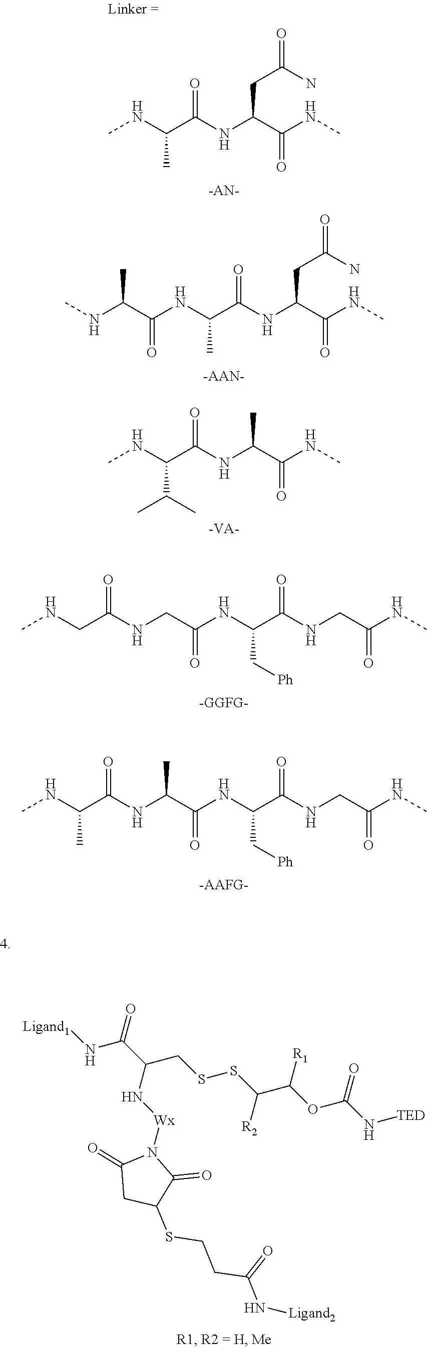

In each formula, the definitions of Ligand₁ and Ligand2 can also be the same as those of R_{P1} and R_{P2}.

Some exemplar ACTEDs are as follows

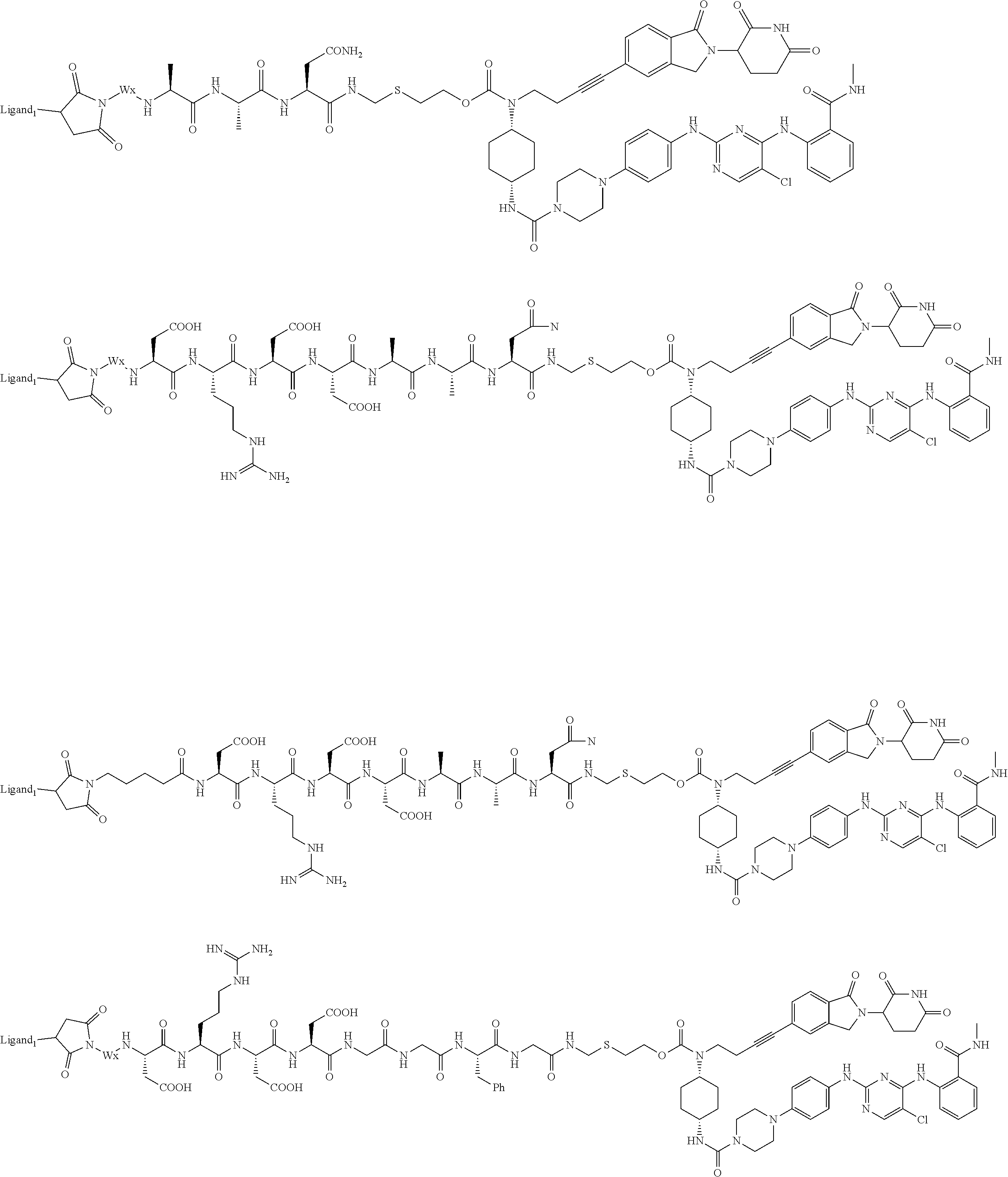

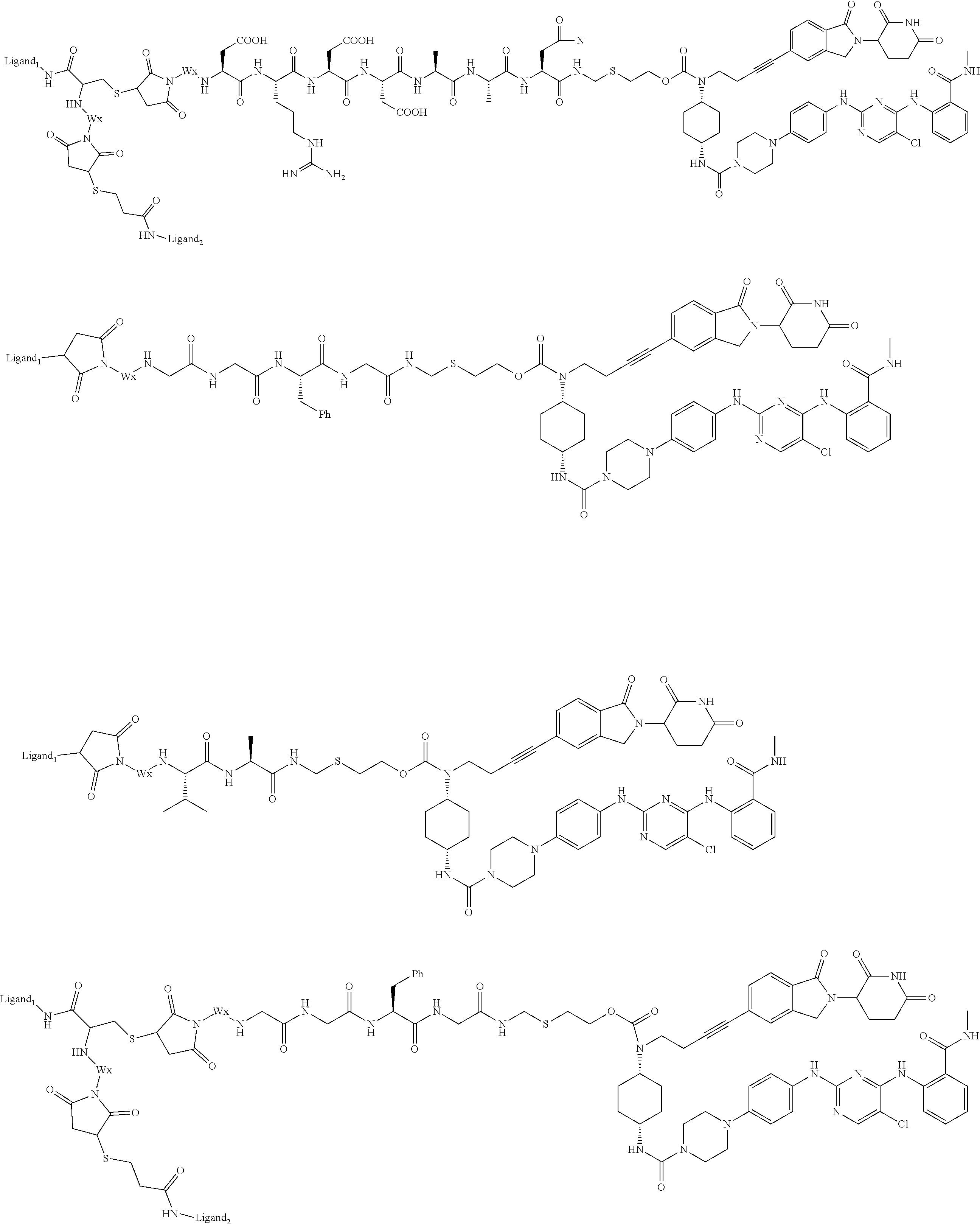

In each formula, the definitions of Ligand₁ and Ligand₂ are the same as those of R_{P1} and R_{P2}, respectively.

### Polypeptide Element

As used herein, the term “polypeptide element” includes peptide fragments (such as oligopeptide comprising 3-20 aa) or proteins. In addition, this term also includes intact proteins or fragments thereof. Preferred polypeptide elements include antibodies (such as intact antibodies, single-chain antibodies, nanobodies, antibody fragments), especially those antibodies that against tumor cell markers (such as tumor markers located on the surface of tumor cells, such as receptors on the cell surface) or inflammatory factors (such as inflammatory factors associated with autoimmune diseases).

As used herein, the term “antibody” or “immunoglobulin” is a heterotetrameric glycoprotein of about 150,000 daltons with the same structural characteristics, which consists of two identical light chains (L) and two identical heavy chains (H). Each light chain is connected to the heavy chain by a covalent disulfide bond, and the number of disulfide bonds between the heavy chains of different immunoglobulin isotypes are different. Each heavy and light chain also has regularly spaced intrachain disulfide bonds. Each heavy chain has a variable region (VH) at one end, followed by multiple constant regions. There are a variable region (VL) at one end of each light chain and a constant region at the other end. The constant region of the light chain is opposite to the first constant region of the heavy chain, and the variable region of the light chain is opposite to the variable region of the heavy chain. Special amino acid residues form an interface between the variable regions of the light chain and the heavy chain.

As used herein, the terms “single-domain antibody” and “nanobody” have the same meaning, and refer to cloning the variable region of the heavy chain of an antibody, and constructing a single-domain antibody consisting of only one heavy chain variable region, which is the smallest antigen-binding fragment that having complete functions. Usually, after obtaining an antibody naturally missing the constant region 1 (CH1) of the light chain and the heavy chain, the variable region of the antibody heavy chain is cloned to construct a single domain antibody consisting of only one heavy chain variable region.

As used herein, the term “variable” means that certain parts of the variable region of the antibody are different in sequence, which forms the binding and specificity to specific antigens of various specific antibodies. However, variabilities are not evenly distributed throughout the variable regions of the antibodies. It is concentrated in three fragments that are called complementarity determining regions (CDR) or hypervariable regions in the variable regions of light chain and heavy chain. More conservative parts of the variable region are called the framework region (FR). The variable regions of the natural heavy and light chains each contain four FR regions, which are in a roughly f-folded conformation and are linked by three CDRs that form a linking loop, which in some cases can form a partially folded structure. The CDRs in each chain are closely placed together through the FR regions and form the antigen binding site of the antibody together with the CDRs in other chain (see Kabat et al., NIH Publ. No. 91-3242, Volume I, pages 647-669 (1991)). Constant regions do not directly participate in the binding of antibodies to antigens, but they exhibit different effector functions, such as participating in antibody-dependent cytotoxicity of antibodies.

The “light chains” of vertebrate antibodies (immunoglobulins) can be classified in one of two distinct categories (called x and X) based on the amino acid sequence of constant regions thereof. According to the amino acid sequence of the constant region in heavy chain thereof, immunoglobulins can be classified into different types. There are five main classes of immunoglobulins: IgA, IgD, IgE, IgG and IgM, some of which can be further classified into subclasses (isotypes), such as IgG1, IgG2, IgG3, IgG4, IgA and IgA2. The constant regions in heavy chains corresponding to different classes of immunoglobulins are called α, δ, ε, γ, and μ, respectively. The subunit structures and three-dimensional configurations of different classes of immunoglobulins are well known to those skilled in the art.

Generally, the antigen-binding properties of antibodies can be described by 3 specific regions located in the variable regions of the heavy and light chains, called variable regions (CDR), which are divided into 4 framework regions (FRs). The amino acid sequence of 4 FRs is relatively conservative and does not directly participate in the binding reaction. These CDRs form a loop structure, and the O-pleated sheet formed by the FRs in between are close to each other in space structure, and the CDRs on the heavy chain and the corresponding CDRs on the light chain constitute the antigen binding site of the antibody. It can be determined by comparing the amino acid sequences of antibodies of the same type which amino acids constitute the FR or CDR regions.

In the present invention, the polypeptide elements can include not only intact antibodies, but also fragments of antibodies with immunological activity (such as Fab or (Fab′)₂ fragment; heavy chain of antibodies; or light chain of antibodies) or fusion proteins formed by antibodies and other sequences. Therefore, the present invention also includes fragments, derivatives and analogs of the antibodies.

### Targeting Ligand

Targeting ligands (or moiety of target protein or target protein ligand or ligand) are small molecules that capable of binding to interesting target protein.

Some embodiments of this application relate to target molecules. Representative target molecules include but are not limited to folic acid, Hsp90 inhibitors, kinase inhibitors, MDM2 inhibitors, compounds targeting proteins containing human BET bromodomain, compounds targeting cytoplasmic signaling protein FKBP12, HDAC inhibitors, human lysine methyltransferase inhibitors, angiogenesis inhibitors, immunosuppressive compounds and compounds targeting aryl hydrocarbon receptor (AHR).

In certain embodiments, the targeting ligand is capable of binding kinases, BET bromodomain-containing proteins, cytoplasmic signaling proteins (such as FKBP12), nucleoproteins, histone deacetylases, lysine methyl transferase, proteins regulating angiogenesis, proteins regulating immune response, aromatic hydrocarbon receptors (AHRs), estrogen receptors, androgen receptors, glucocorticoid receptors, or transcription factor (e.g., SMARCA4, SMARCA2, TRIM24).

In certain embodiments, kinases, to which targeting ligands are able to bind, include, but are not limited to Tyrosine kinases (for example, AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFR3, FGFR4, FGR, FLT1, FLT3, FLT4, FRK, FYN, GSG2, HCK, HRAS, HSP90, IGF1R, ILK, INSR, INSRR, IRAK4, ITK, JAK1, JAK2, JAK3, KDR, KIT, K^{RA}S, KSP, KSR1, LCK, LMTK2, LMTK3, LTK, LYN, MATK, MERTK, MET, MLTK, MST1R, MUSK, NPR1, N^{RA}S, NTRK1, NTRK2, NTRK3, PDGF^{RA}, PDGF^{RB}, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TNK2, TNNI3K, TXK, TYK2, TYRO3, YES1 or ZAP70), Serine/threonine kinase (such as Casein Kinase 2, protein kinase A, protein kinase B, protein kinase C, ^{Ra}f kinase, CaM kinase, AKT1, AKT2, AKT3, ALK1, ALK2, ALK3, ALK4, Auro^{ra} A, Auro^{ra} B, Auro^{ra} C, CHK1, CHK2, CLK1, CLK2, CLK3, DAPK1, DAPK2, DAPK3, DMPK, ERK1, ERK2, ERK5, GCK, GSK3, HIPK, KHS1, LKB1, LOK, MAPKAPK2, MAPKAPK, MEK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PIM2, PLK1, RIP2, RIP5, RSK1, RSK2, SGK2, SGK3, SIK1, STK33, TAO1, TAO2, TGF-β, TLK2, TSSK1, TSSK2, MLK1 or MLK2), cyclin-dependent kinases (such as Cdk1-Cdk11) and Leucine-rich repetitive kinase (such as LRRK2).

### Target Molecule

In the conjugates of formula I of the present application, the conjugate binds to target proteins through R^{T} (the moiety of target molecule).

In the present invention, the target molecule can be target molecule A, target molecule T, or the combination thereof.

In the present invention, the target molecule can be any inhibitor of the target protein. The target molecule can be a highly effective inhibitor of the target protein, or an inhibitor with relatively poor activity. Specifically, the target molecule of the present invention may be a small molecule inhibitor known in the art against any target protein in the art.

In some embodiments, the target molecule used herein has a radical, such as —O—, —NR^{a}— (wherein R^{a} is H, or substituents such as C1-C6 alkyl, —CO—, —COO—, and the like), that is able to connect to a linker molecule of the present invention (e.g. L1 in the present invention) monovalently to form an ether, an amine, an amide and the like, thereby forming the moiety of target molecule.

The target protein may be a variety of target proteins known in the art, representative examples include, but are not limited to MDM2, AKT, BCR-ABL, Tau, BET (BRD2, BRD3, BRD4), ERRα, FKBP12, RIPK2, E^{RB}B3, androgen receptor, MetAP2, TACC3, FRS2α, PI3K, DHFR, GST, Halo Tag, C^{RA}BPI, C^{RA}BPII, ^{RA}R, aromatic hydrocarbon receptor, estrogen receptor. Different target proteins and some corresponding inhibitors can be obtained commercially or prepared by conventional methods. For example, as for MDM2, the inhibitors thereof can be referred to documents such as WO 2017176957, WO2017176958A1.

In another specific embodiment, R_{T} is selected from Table B1 or Table B2

TABLE B1

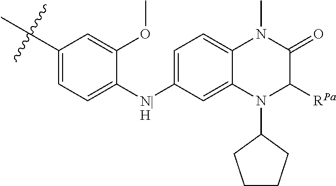

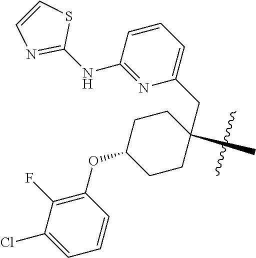 [SMILES: C[C@]1(Cc2cccc(Nc3nccs3)n2)CC[C@@H](Oc2cccc(Cl)c2F)CC1]

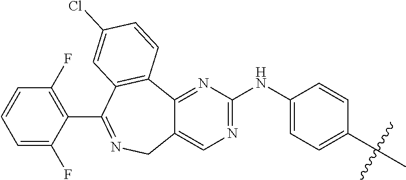 [SMILES: *c1ccc(Nc2ncc3c(n2)-c2ccc(Cl)cc2C(c2c(F)cccc2F)=NC3)cc1]

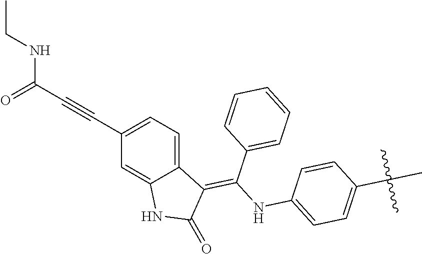 [SMILES: *c1ccc(N/C(=C2\C(=O)Nc3cc(C#CC(=O)NCC)ccc32)c2ccccc2)cc1]

TABLE B2

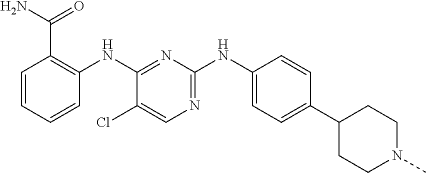

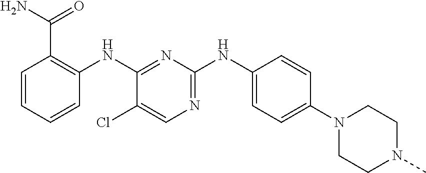

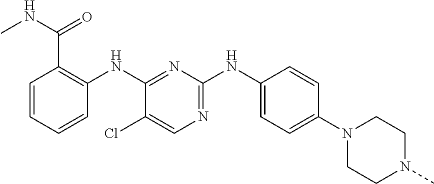

In each formula, R^{Pa} is selected from the group consisting of: optionally substituted C₁₋₆alkyl, optionally substituted C₂₋₆alkenyl, optionally substituted C₂₋₆alkynyl.

In another preferred embodiment, formula P1 is as shown in any of the following

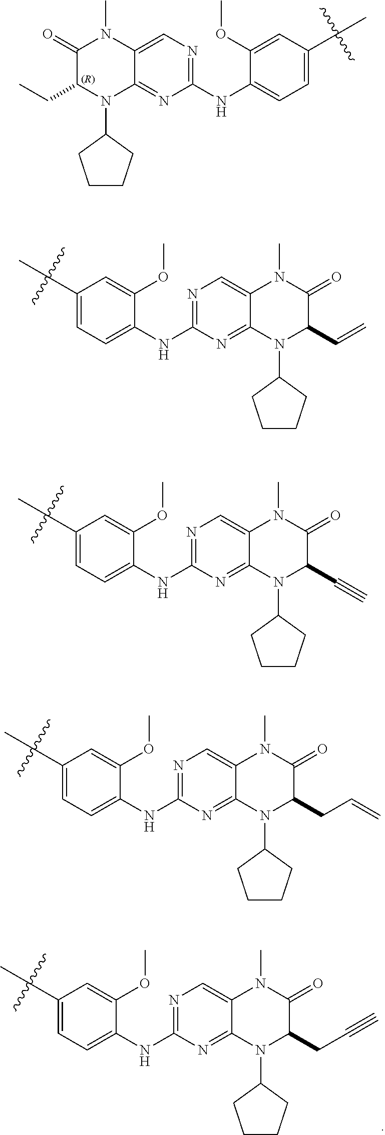

### E3 Ligase Ligand

In the present invention, the moiety of E3 ligase ligand (R_{E3}) is used for binding E3 ligase.

In a specific embodiment, representative moieties of E3 ligase ligand have a structure of formula A1 or A2:

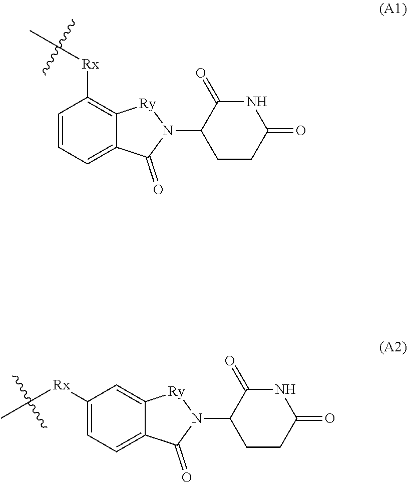

In formula A, R_{X} is selected from null, C1-C6 alkyl, C2-C6 alkenyl, C2-C6 alkynyl, O, NH, S, CO and SOₙ (n is 1 or 2) and the like; R_{Y} is CH₂, C═S, CO; and the E3 ligase ligand (R_{E3} in formula I) is able to connect to L1 of the present invention via R_{X} group in the E3 ligase ligand, such as —Rₓ-L1-R_{T} (such as —O-L1-R_{T});
or, representative moieties of E3 ligase ligand have a structure of formula A1b:

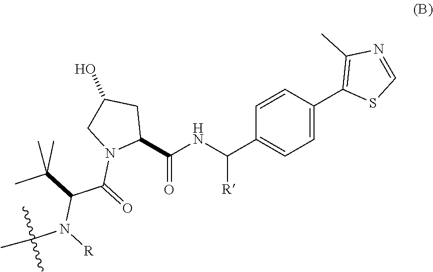

in formula A1b, R′ is H or C1-C6 alkyl (such as Me), R is H, or C1-C6 alkyl (such as Me or Et).

In some embodiments, the E3 ligase ligand used herein has a radical, such as —O—, —NR^{a}— (wherein R^{a} is H, or substituents such as C1-C6 alkyl and the like, —CO—, —COO—, and the like), that is able to connect to a linker molecule of the present invention (e.g. L1 in the present invention and the like) monovalently to form an ether, an amine, an amide and the like.

In another specific embodiment, R_{E3} (moiety of E3 ligase ligand) used herein is selected from Table C:

TABLE C

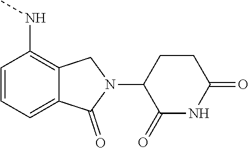

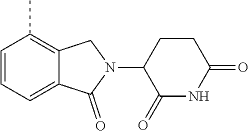

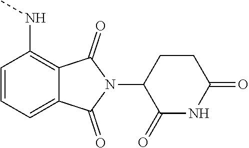

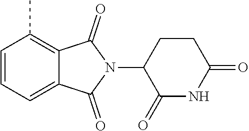

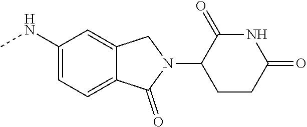

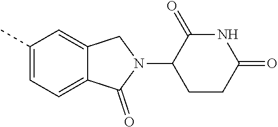

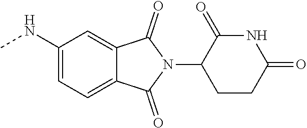

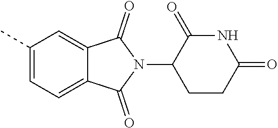

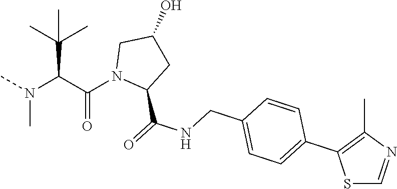

In another preferred embodiment, R_{E3} is of formula A1.2 or formula A2.2.

### Linker Molecule (L1 as Described Herein)

The linker molecules of the present invention are used for connecting the target molecule and the E3 ligase ligand. For example, it can be connected to the target molecule or the E3 ligase ligand through functional groups at both ends (such as —OH, —SH, —NH₂, —NHR, —SOOH or —COOH); wherein R is selected from: substituted or unsubstituted C1-C10 alkyl, —(C═O)—R′, (C═O)NH—R′, —NH(C═O)—R′, —SO₂—R′, —NHSO₂—R′, —SO₂NH—R′, —SO—R′, —NHSO—R′, —SONH—R′, —PO₃—R′, —NHCOO—R′, —COO—R′ or —NH—CO—NH—R′, —NH—CO—O—R′ or —X′-L3-Z; where L3 is a linking group, and Z is a polypeptide element (such as a ligand, antibody or peptide fragment, etc.) or a targeting molecule such as a small molecule with targeting function (such as folic acid, HSP90 inhibitors, etc.).

### Linker and Coupling Method

The linker L1 of the present invention is used for connecting the target molecule (moiety) R_{T} and the E3 ligase ligand (moiety) R_{E3}.

Preferably, the target molecule (moiety) or the E3 ligase ligand (moiety) can be connected with the linker through —O—, —S—, —NH—, —NR—, —(C═O)—, —(C═O)O—, —SO₂— and other groups.

The linker of the present invention may further contain a varity of other functional groups, such as —OH, —NHR, —SH and the like.

Typically, the linker L1 of the present invention can be represented by the following general formula II:

—W¹-L2-W²— formula II

In the formula, the definition of W¹, L2, and W² are as described in the first aspect of the present invention.

In another preferred embodiment, W¹ and W² are each independently divalent groups formed by the loss of 1 hydrogen atom forming bivalence from the following monovalent groups: —OH, —NH₂, —SH, —COOH, —SO₂H and the like. For example, the linker and the target molecule can be connected through the linker group shown as below:

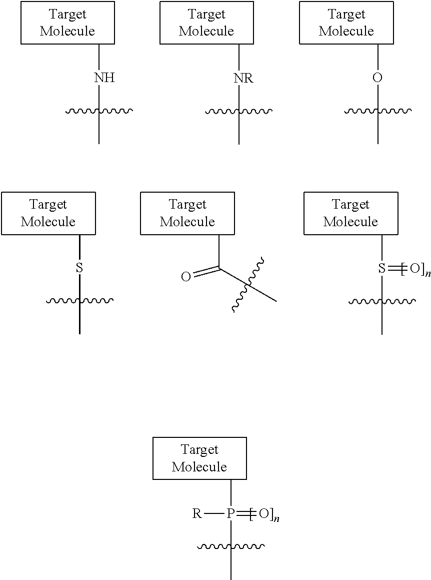

alternatively, W¹ and W² each independently comprise a divalent linking group having a rigid portion (e.g., a portion of 4-membered, 5-membered, or 6-membered aliphatic ring (saturated carbocyclic ring), or a portion of 5-membered or 6-membered aromatic heterocyclic ring, etc.), exemplary examples of which are shown below and in examples.

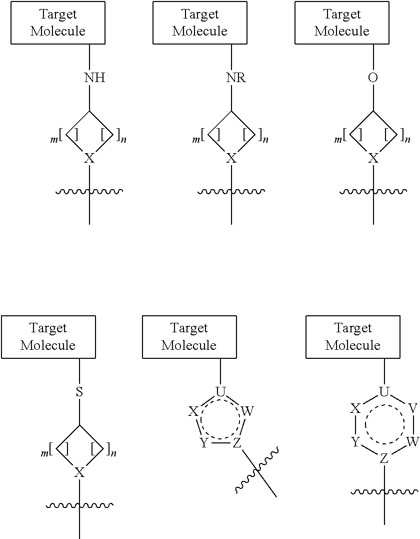

wherein, R in the each of the above formulas is defined as above; n is 1 or 2 or 3.

In a specific embodiment, W¹ and W² are each independently selected from the group consisting of
null, —N(R^{a})—, —C(R^{b})₂—, —N(R^{a})—C(R^{b})₂—, —C(O)—, —C(O)—N(R^{a})—, —C(R^{b})₂—C≡C—, —C≡C—, —C(O)—C≡C—, —CH(OH)—C≡C—, —O—, —S—, —SO₂—, —SO—, —PO₃—, —C(R^{b})═C(R^{b})—, substituted or unsubstituted C3-8 cycloalkyl, substituted or unsubstituted 4 to 10 membered heterocycloalkyl, substituted or unsubstituted C6-10 aryl, substituted or unsubstituted 5 to 10 membered heteroaryl.

### Active Ingredients

As used herein, the term “compound of the invention” refers to the compound or the conjugate of formula I. The term also comprises the crystal forms, or pharmaceutically acceptable salts of the compound of formula (I).

Specifically, the present invention provides a class of conjugates of formula I that are suitable for further attaching with the polypeptide elements (e.g., an antibody, a protein ligand, etc.) or target molecule T, or that are coupled with polypeptide elements or target molecule T;

R_{T}-L1-R_{E3} (I)
wherein R_{L} is a moiety of E3 Ligase Ligand; R_{T} is a moiety of target molecule, and L1 is a linker connecting R_{T} and R_{E3}.

Preferably, R_{L}, R_{T} and L1 are defined as above.

In a specific embodiment, the conjugate provided by the present invention that is suitable for further attaching with the polypeptide elements or the target molecule T is of Formula IV;

R_{T}—W¹-L6-W²—R_{E3} (IV)
wherein R_{T}, R_{E3}, W¹, W² and L7 are defined as above.

In a specific embodiment, the conjugate provided by the present invention that is attached with polypeptide elements or target molecule T is of Formula V;

R_{T}—W¹-L7-W²—R_{E3} (V);
wherein R_{T}, R_{E3}, W¹, W² and L7 are defined as above.

In a specific embodiment, the present invention further provides a conjugate as shown in R_{T}—W¹-L5-Wb-C≡C—R_{E3} (1-1), R_{T}—W¹-L5-CO—R_{E3} (1-2), or R_{T}—W¹-L5-CONH—R_{E3} (1-3);
wherein W^{b} is defined as W; W¹, R_{T}, R_{E3} and L5 are defined as above.

In another preferred embodiment, in formula 1-1, W¹ is selected from the group consisting of NH, and O; preferably, W is NH.

In another preferred embodiment, in formula 1-1, W^{b} is selected from the group consisting of null, —CH₂—, —CH(OH)—, and —C(O)—.

In a specific embodiment, the present invention provides a conjugate as shown below;

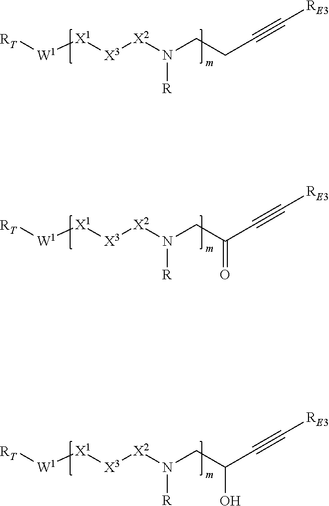

wherein W¹, R_{T}, R_{E3} and R are defined as above; preferably, R is H, C1-6alkyl (such as Me, Et etc.);
m=0, 1, 2, 3 etc. (preferably, m is not 0);
X¹, X² and X³ are each independently selected from 0, C₁₋₄alkylene,

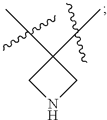 [SMILES: *C1(*)CNC1]

Preferably, W¹ is W, and W is defined as above. More preferably, W¹ is NH.

In a specific embodiment, the present invention further provides a conjugate as shown below;

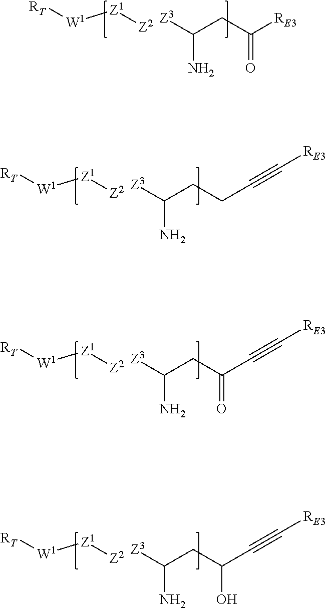

In each formula,
R, R₁, R_{T} and R_{E3} are defined as above;
Z¹, Z² and Z³ are each independently selected from O, C₁₋₄alkylene, —CH(OH)—,

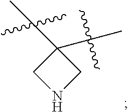 [SMILES: *C1(*)CNC1]

m=0, 1, 2, 3, 4 and other integers.

In another specific embodiment, the conjugate is a conjugate selected from Group 1:

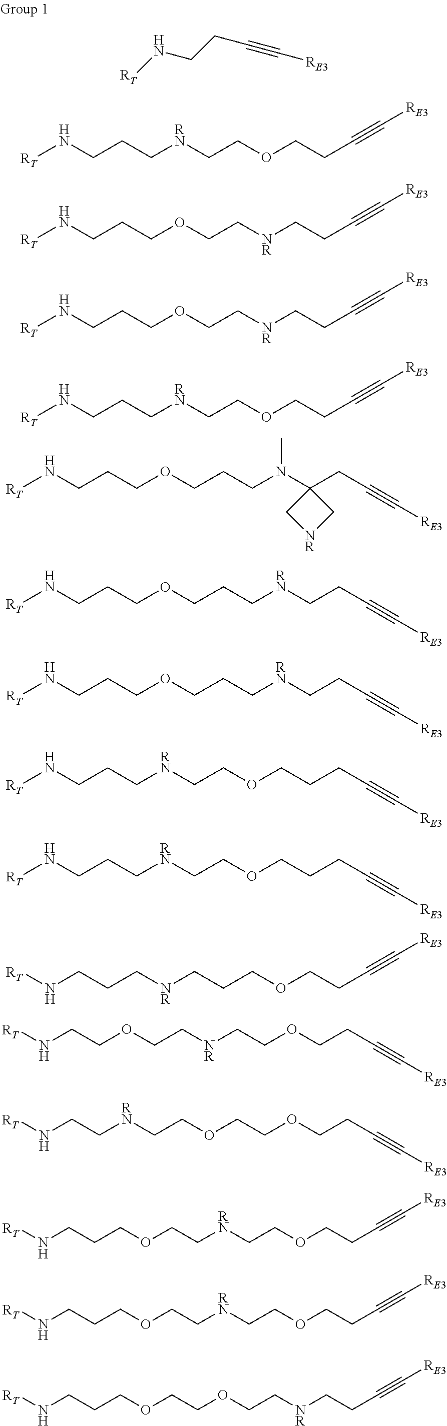

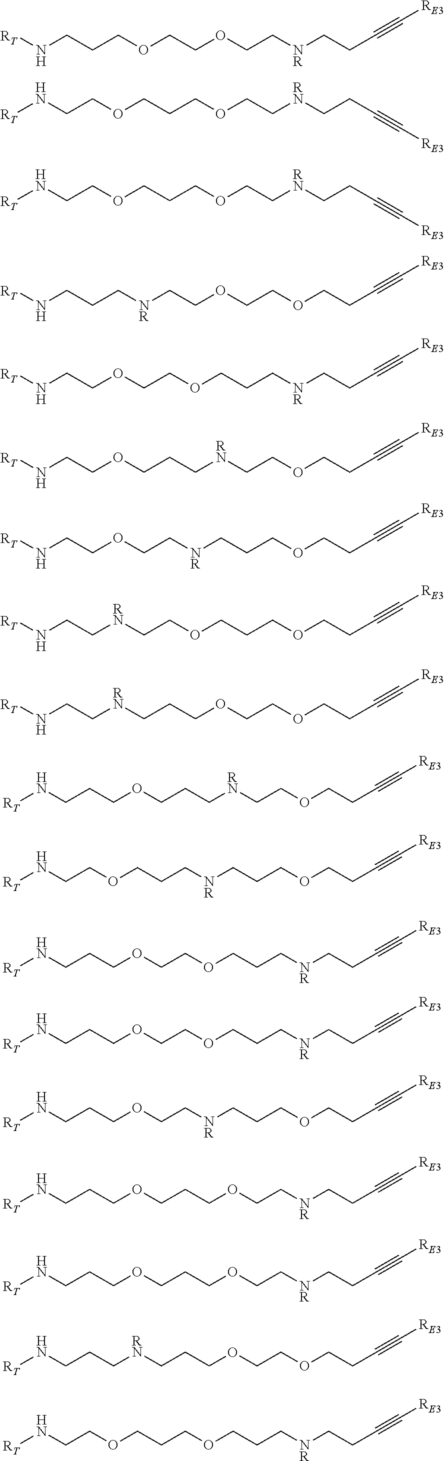

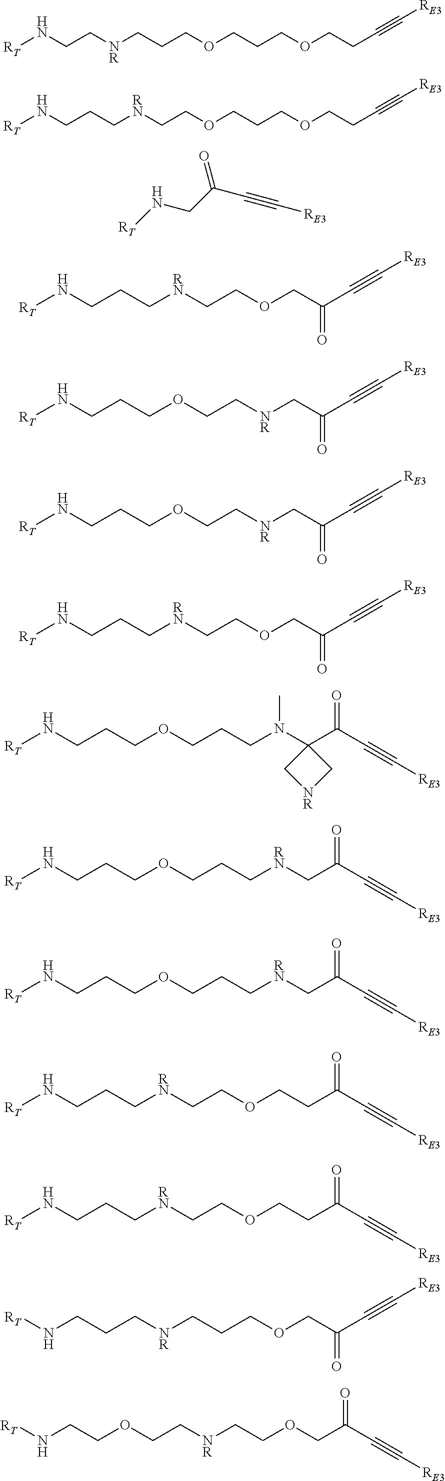

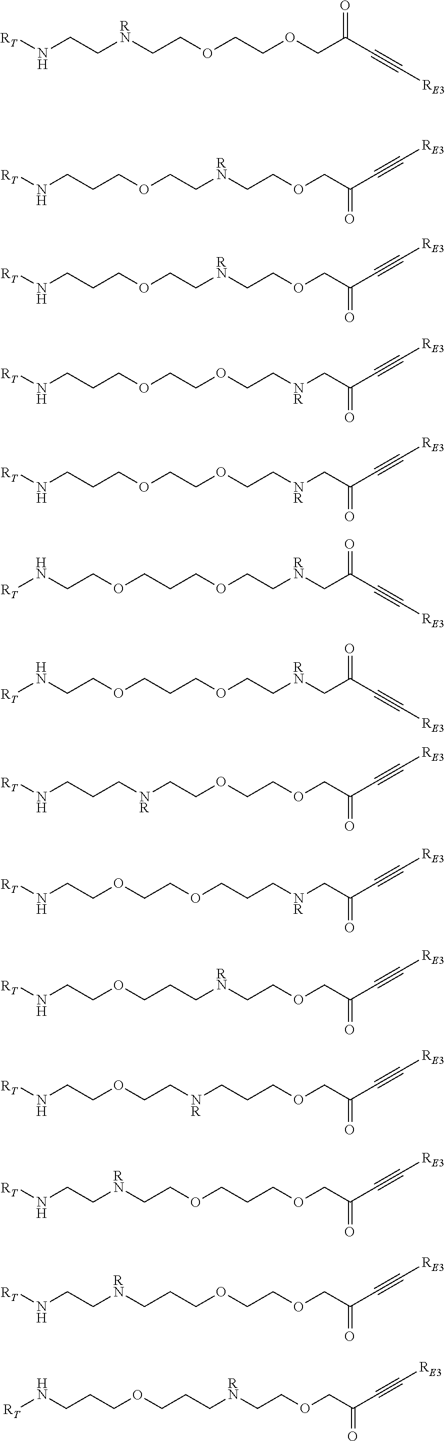

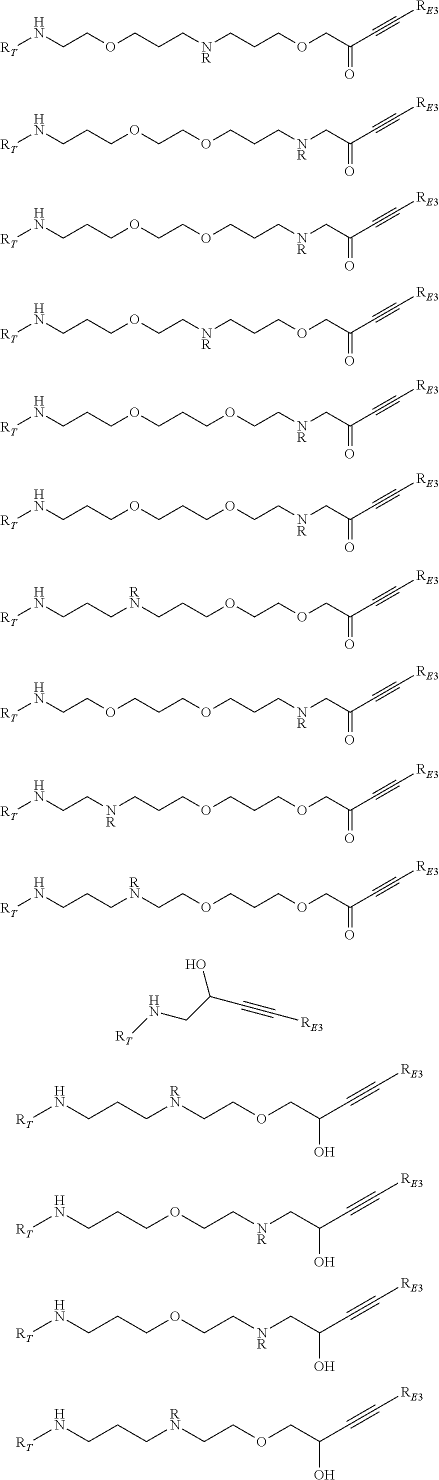

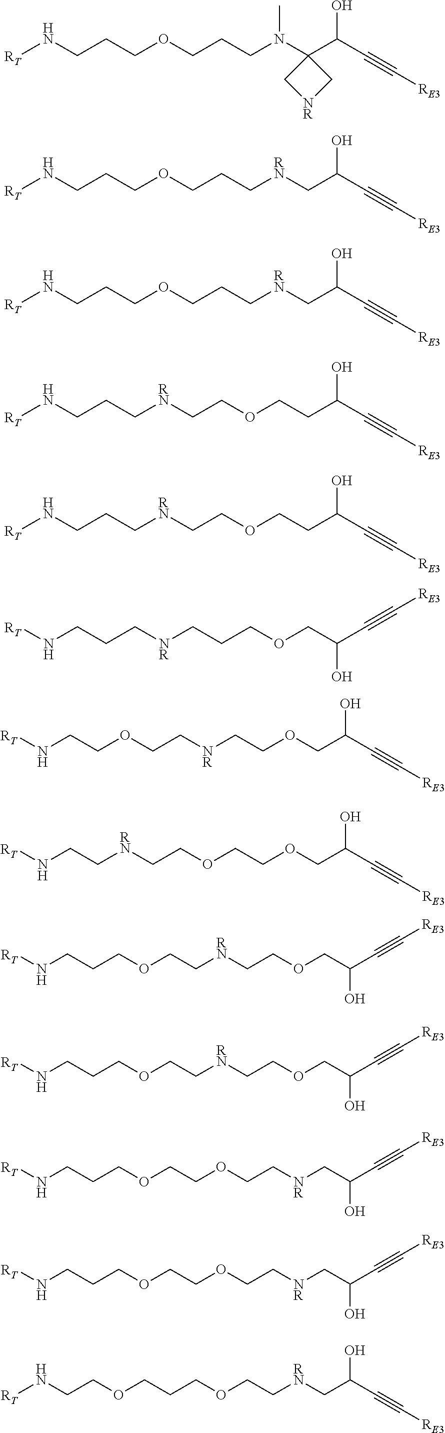

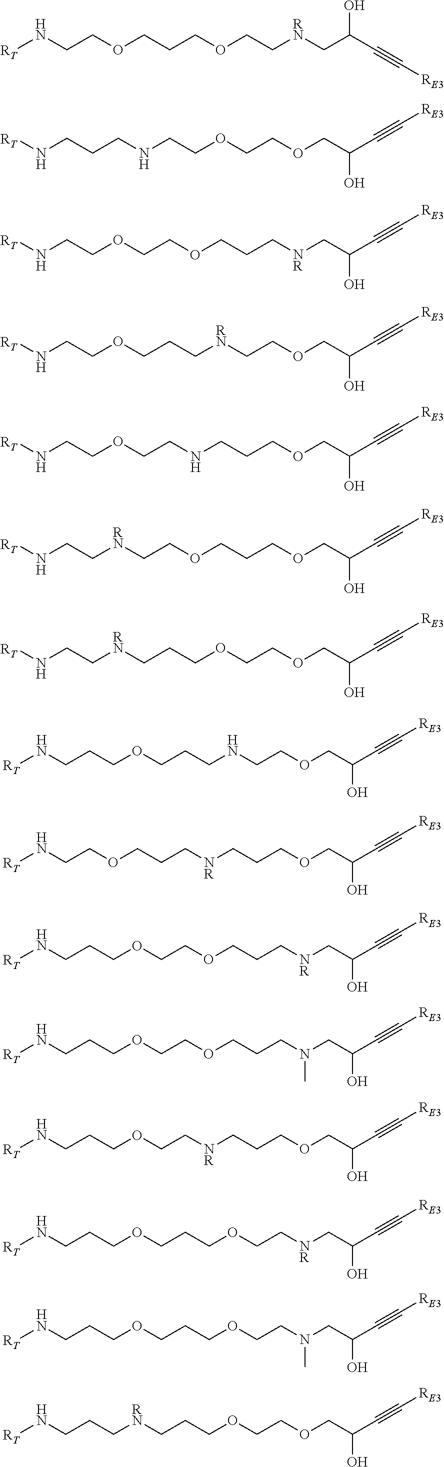

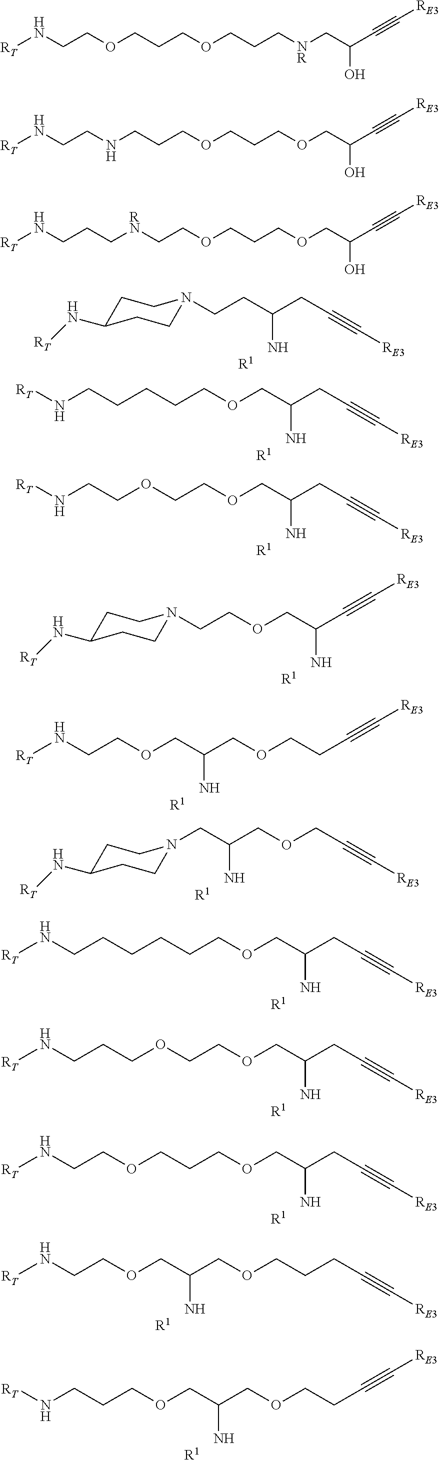

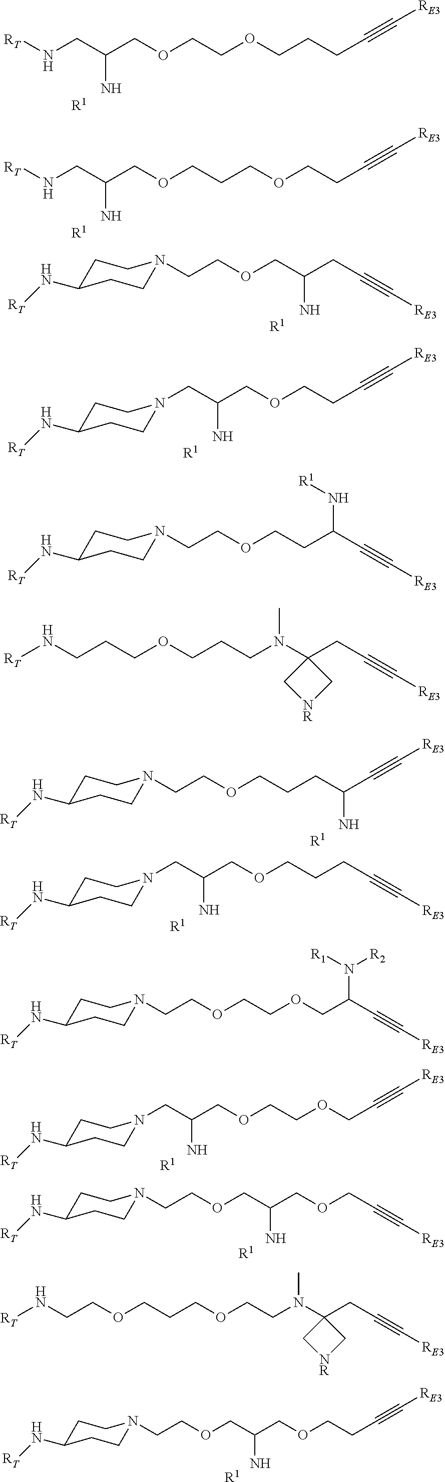

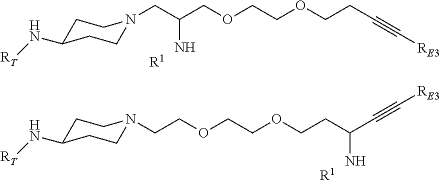

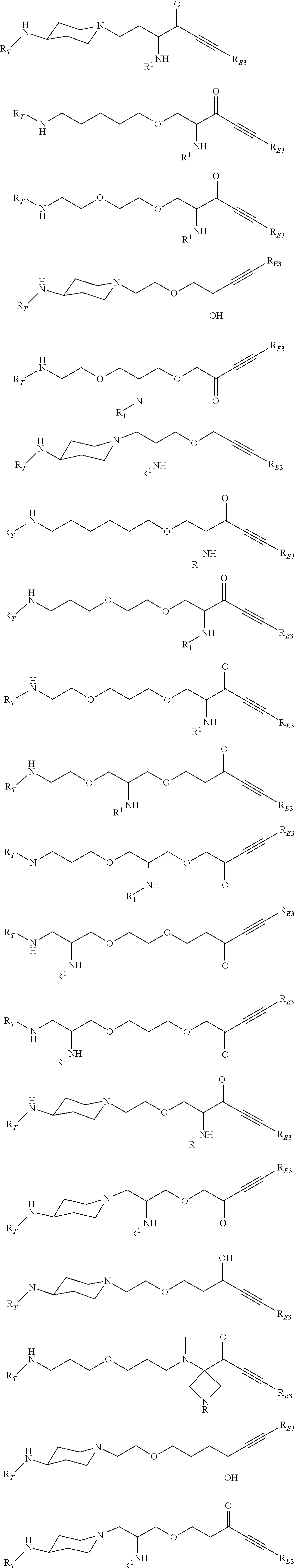

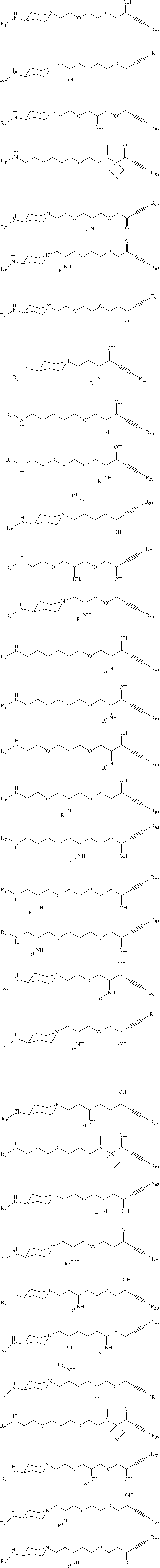

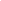

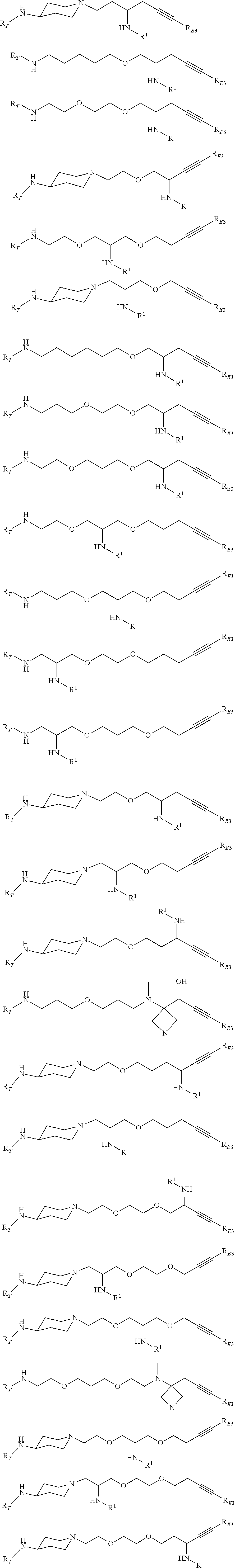

wherein R_{T}, R_{E3}, R and R₁ are defined as above; preferably, R and R¹ are each independently —W³-L3-W⁴—(R_{P})_{q}, wherein W³, L3, W⁴, R_{P} and m are defined as above.

In a specific embodiment, the present invention further provides a conjugate as shown in R_{T}—W¹-L6-W^{b}—C≡R_{E3} (1a-1), R_{T}—W¹-L6-CO—R_{E3} (1 a-2), or R_{T}—W¹-L6-CONH—R_{E3} (1a-3);
wherein W^{b} is defined as W; W¹, R_{T}, R_{E3} and L5 are defined as above.

In a specific embodiment, the present invention further provides a conjugate as shown in R_{T}—W^{a}-L6-W^{b}—C≡C—R_{E3} wherein W^{a} and W^{b} are defined as W; R_{T}, R_{E3} and L6 are defined as above.

In another preferred embodiment, W^{a} is selected from the group consisting of NH, and O; preferably, W is NH.

In another preferred embodiment, W^{b} is selected from the group consisting of null, —CH₂—, —CH(OH)—, and —CO)—.

In another specific embodiment, the conjugate is a conjugate selected from Group 1a:

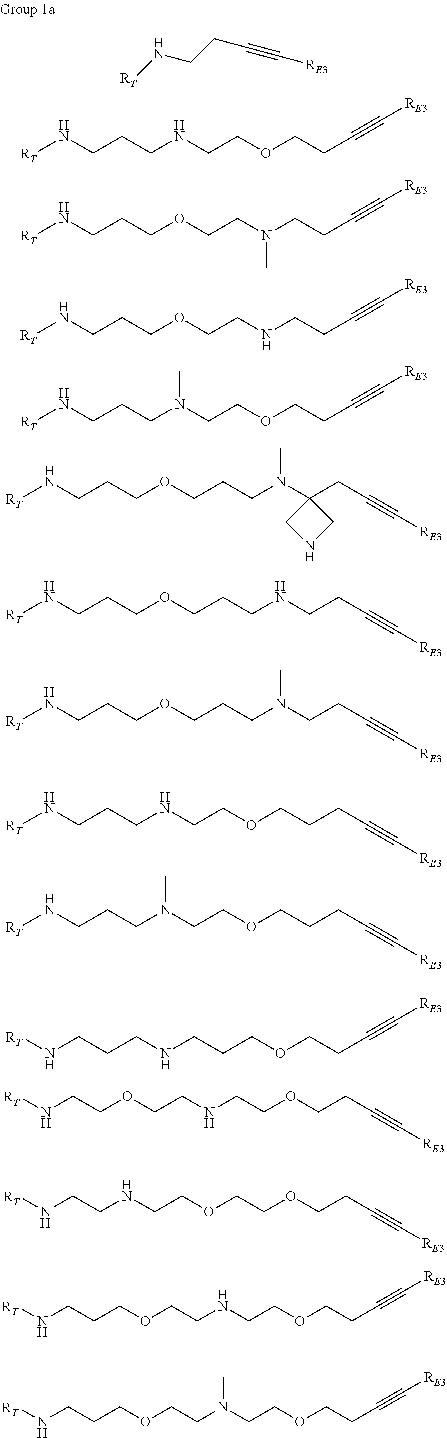

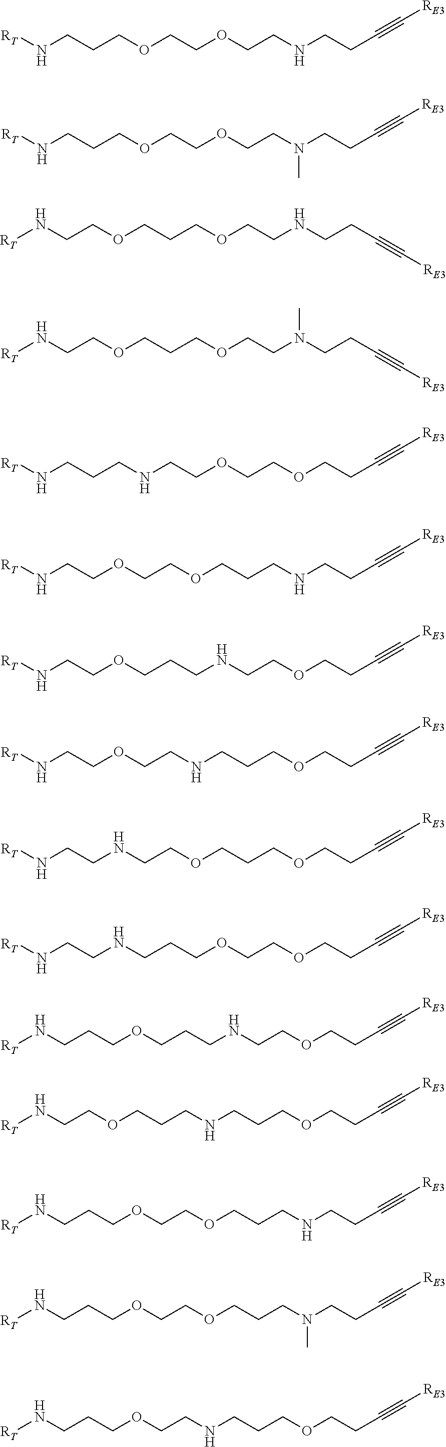

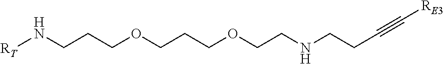

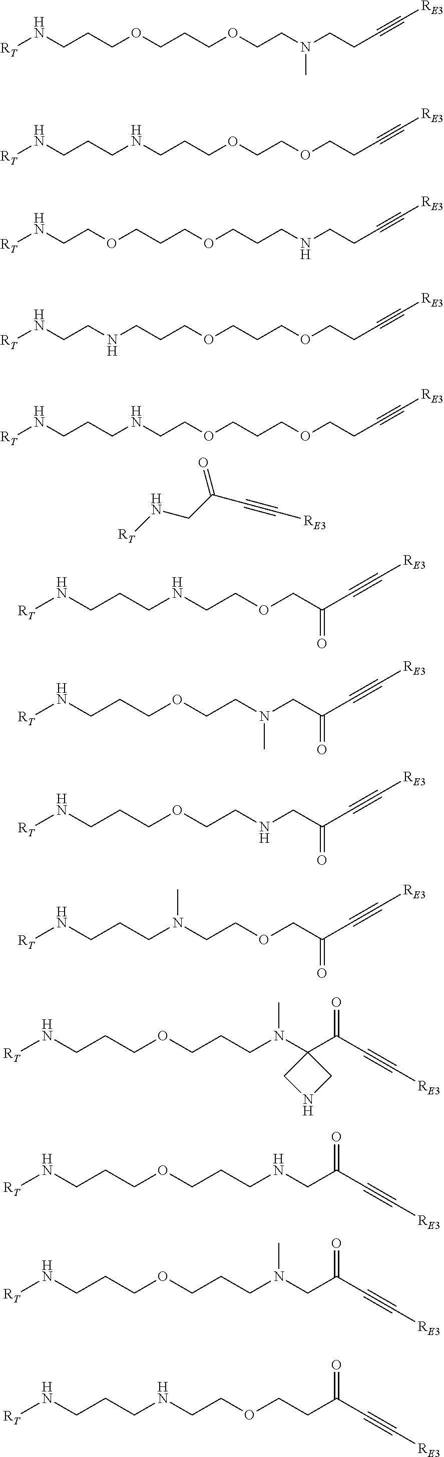

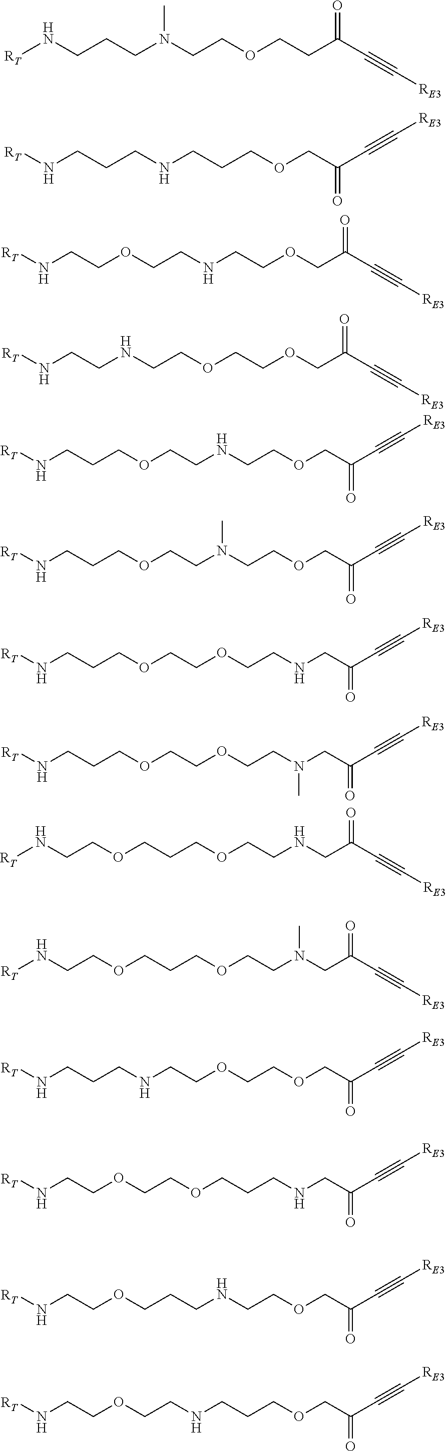

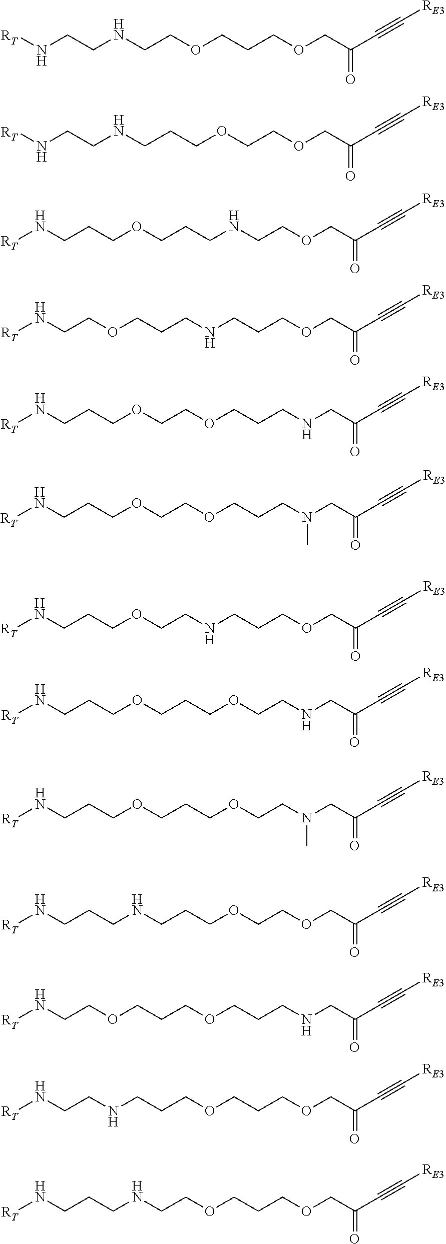

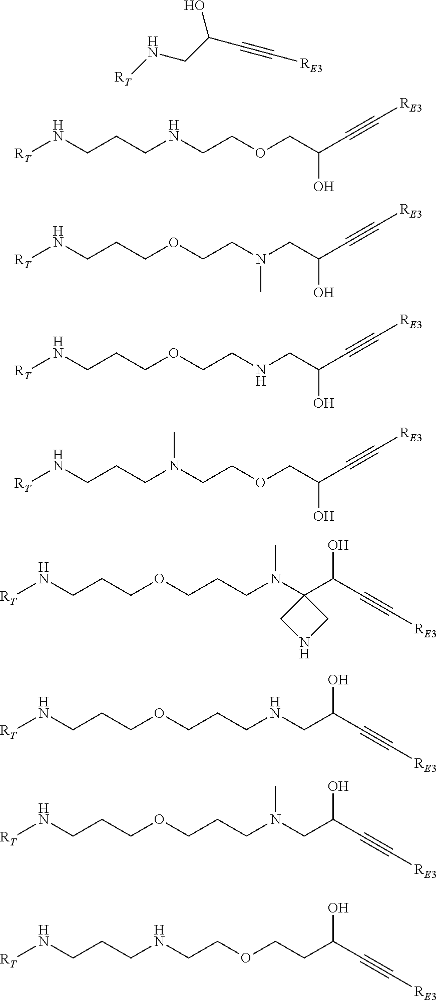

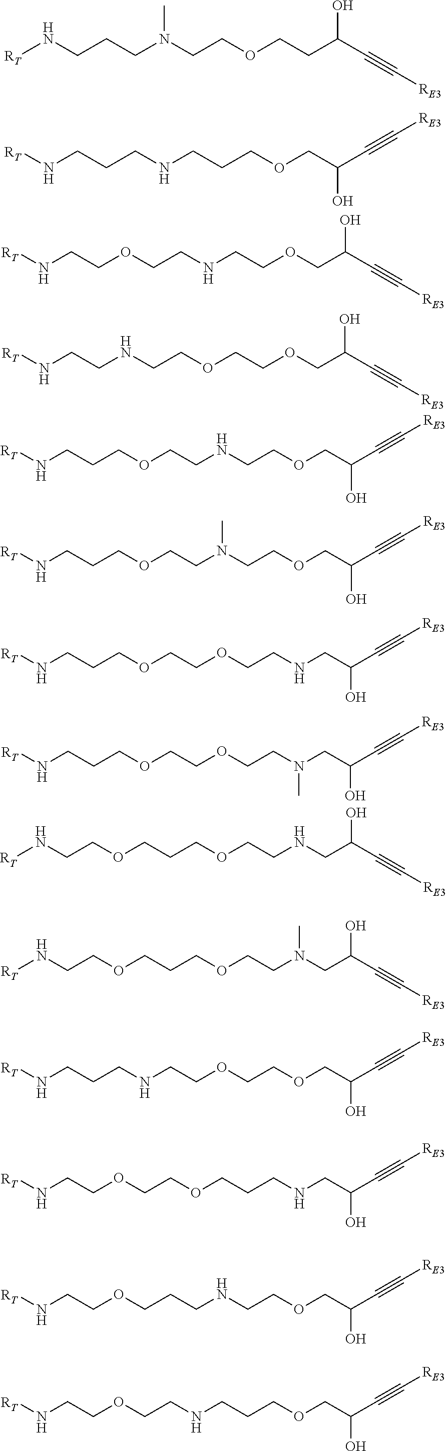

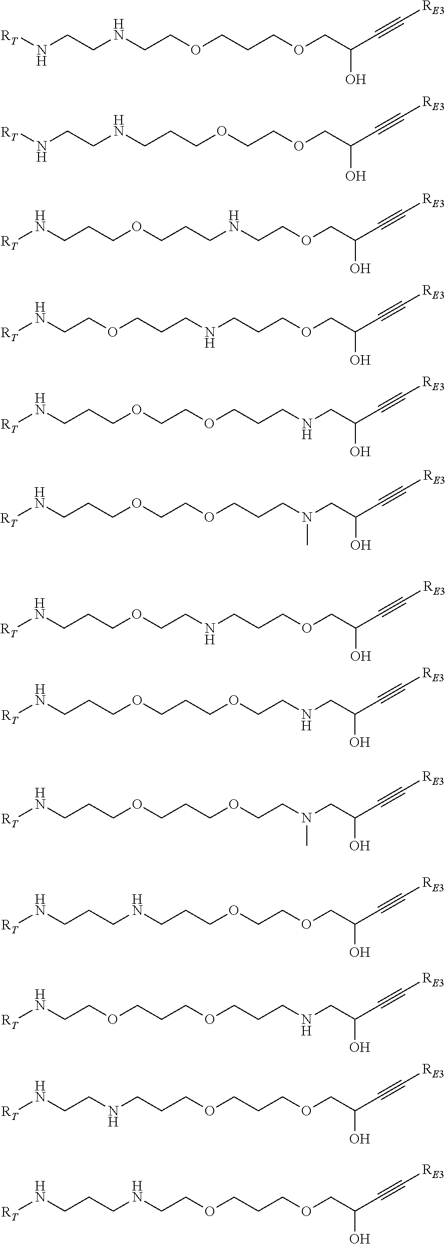

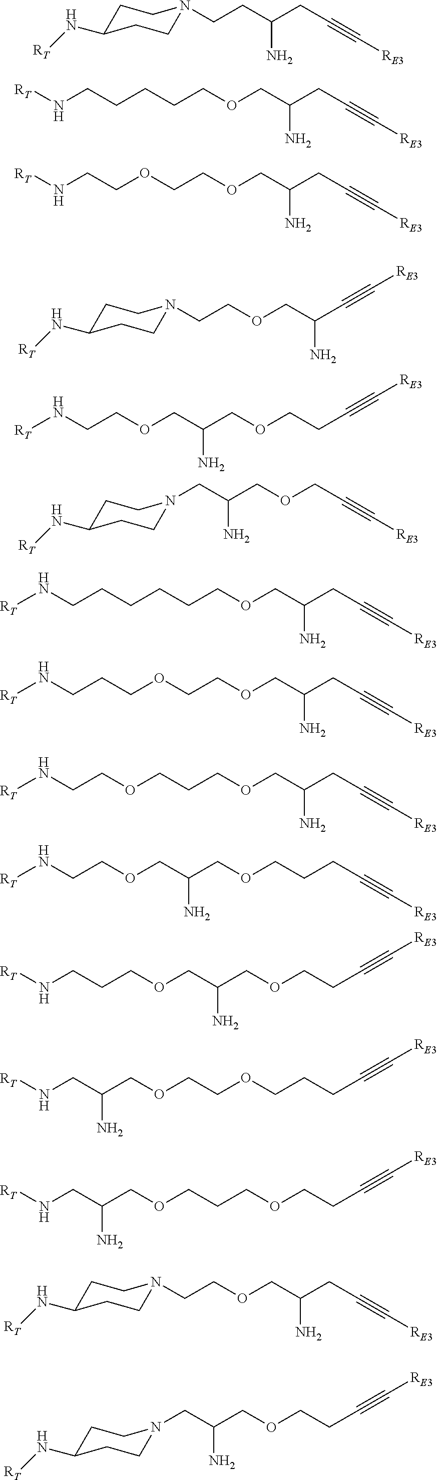

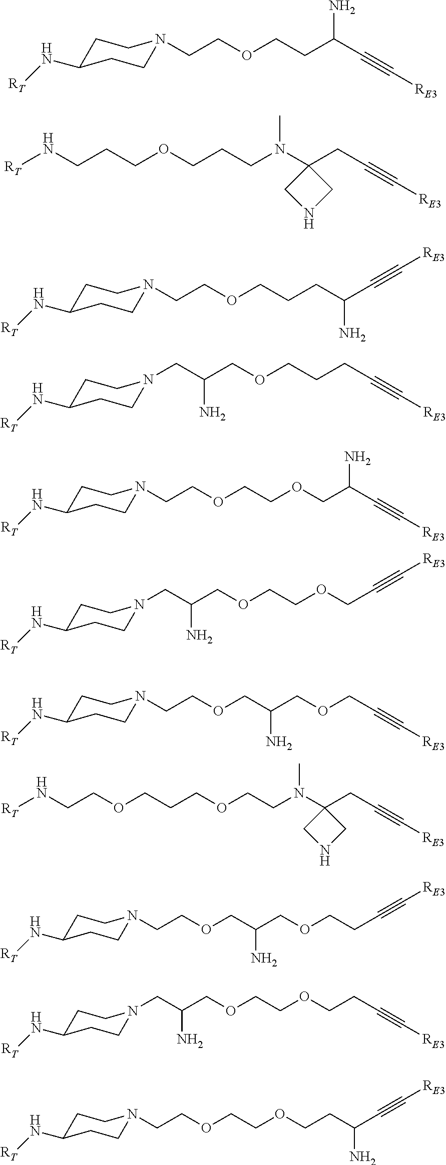

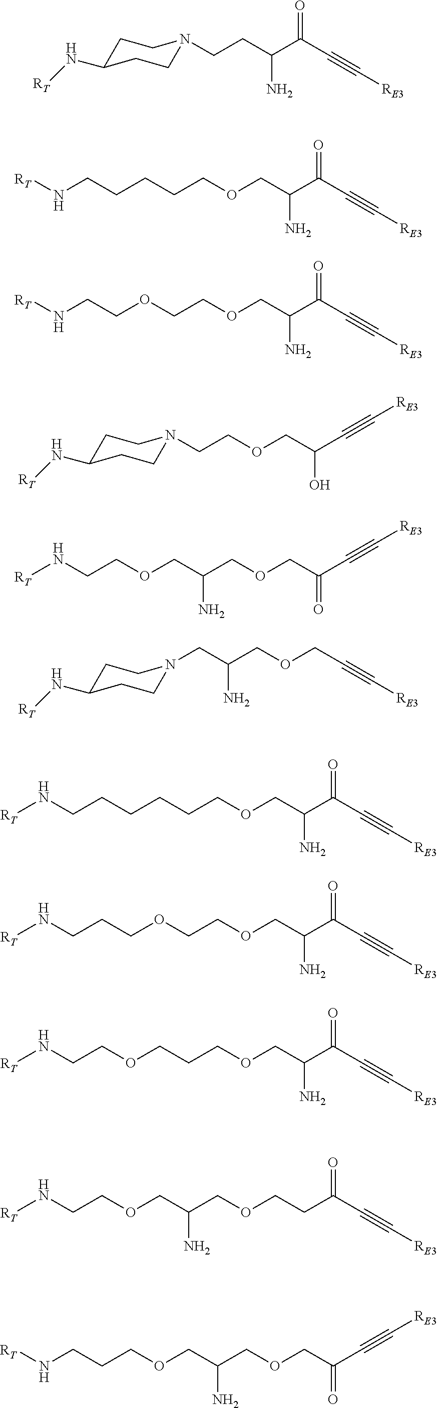

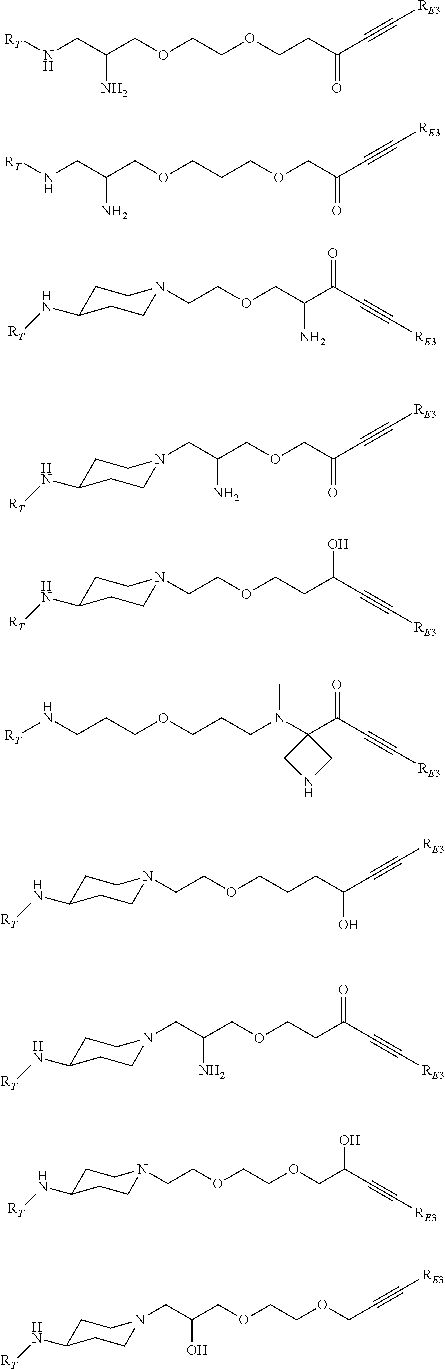

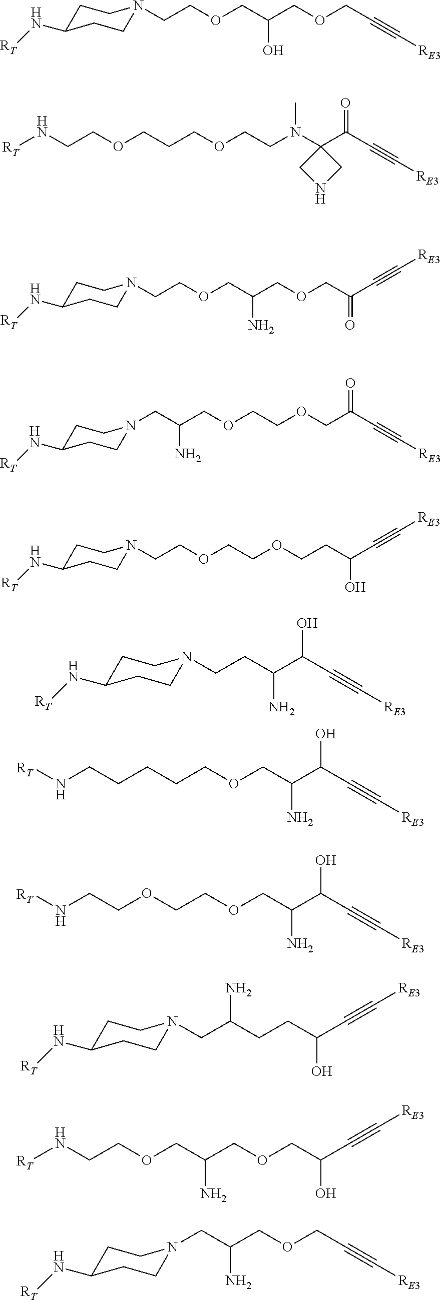

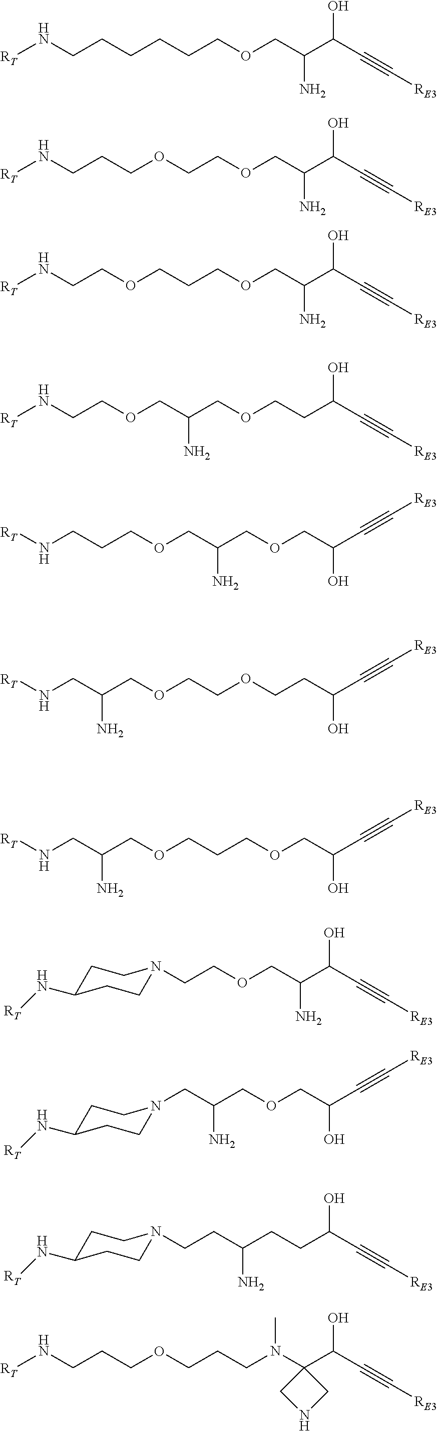

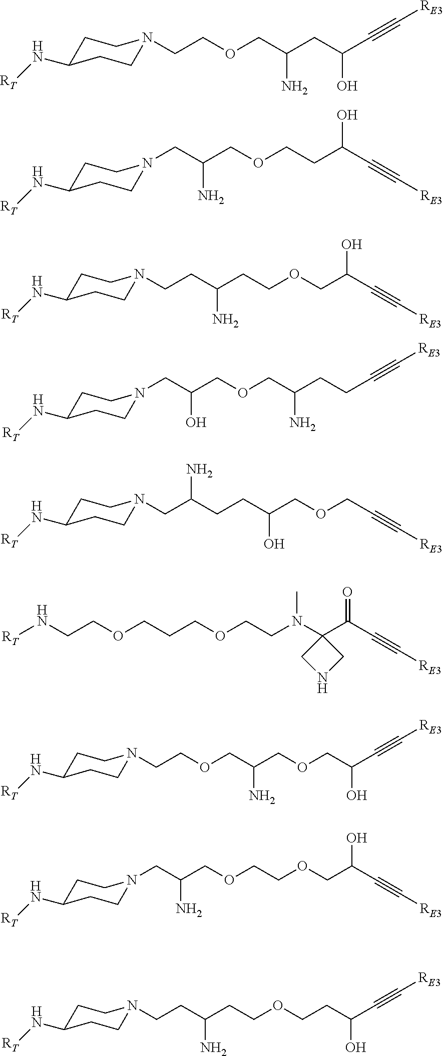

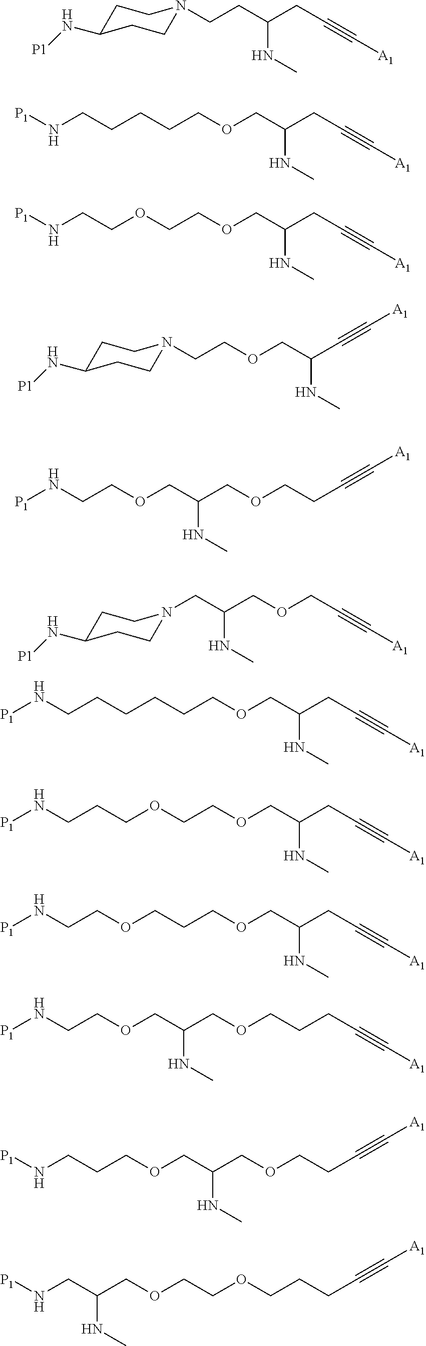

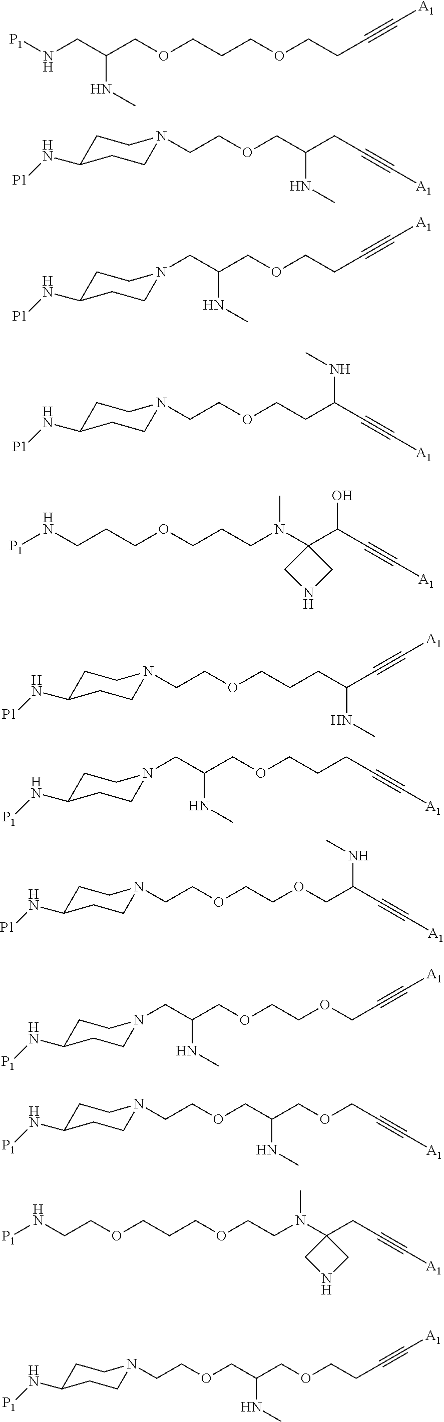

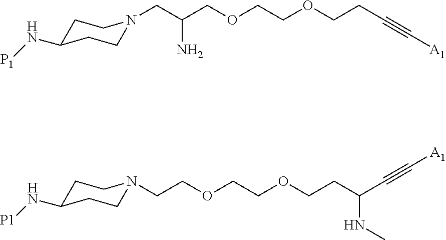

wherein R_{T} and R_{E3} are defined as above.

In a specific embodiment,
the present invention further provides a conjugate as shown in R_{T}—W^{a}—Cr¹—W^{a}—Cr²-L5-W²—R_{E3} (2);
wherein
W^{a} is defined as W;
Cr¹ is null, or C₄₋₇cycloalkyl that is unsubstituted or substituted with C₁₋₄alkyl, or 4 to 6 membered heterocyclyl that is unsubstituted or substituted with C₁₋₄alkyl;
Cr² is 4 to 6 membered heterocyclyl containing nitrogen that is unsubstituted or substituted with C₁₋₄alkyl, and at least one nitrogen heteroatom in Cr² is attached with L5;
W, R_{T}, R_{E3}, W² and L5 are defined as above.

In another preferred embodiment, W² is selected from the group consisting of W^{b}—C≡C, C(O), and C(O)NH.

In another specific embodiment, the present invention further provides a conjugate as shown in R_{T}—W^{a}—Cr¹—Cr²-L5-W^{b}—C≡C—R_{E3};
wherein W^{a} and W^{b} are defined as W;
Cr¹ is null, or C₄₋₇cycloalkyl that is unsubstituted or substituted with C₁₋₄alkyl, or 4 to 6 membered heterocyclyl that is unsubstituted or substituted with C₁₋₄alkyl;
Cr² is 4 to 6 membered heterocyclyl containing nitrogen that is unsubstituted or substituted with C₁₋₄alkyl, and at least one nitrogen heteroatom in Cr² is attached with L5;
R_{T}, R_{E3} and L5 are defined as above.

Preferably, W^{a} is selected from the group consisting of: NH, O; preferably, Wₐ is NH.

Preferably, W^{b} is selected from the group consisting of null, —CH₂—, —CH(OH)—, and —C(O)—.

Preferably, the conjugate is selected from the group consisting of:
R_{T}—NH—Cr¹—Cr²-L5-CH₂—C≡C—R_{E3};
R_{T}—NH—Cr¹—Cr²-L5-C(O)—C≡C—R_{E3};
R_{T}—NH—Cr¹—Cr²-L5-CH(OH)—C≡C—R_{E3};
R_{T}—NH—Cr¹—Cr²-L5-C≡C—R_{E3}; in each formula, R_{T}, R_{E3}, Cr¹, Cr² and L5 are defined as above.

Preferably, the conjugate is selected from the group consisting of:

R_{T}—NH—Cr¹—Cr²-L8-C≡C—R_{E3};
wherein R_{T}, R_{E3}, Cr¹, Cr² and L8 are defined as above.

In another preferred embodiment, Cr¹ is null or

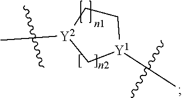

wherein Y¹ are Y² are each independently selected from CH and N; n1=0, 1 or 2; and n2=1 or 2.

In another preferred embodiment, Cr² is

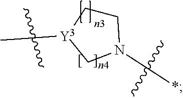

wherein * indicates the position connected with L5; Y³ is selected from CH and N, n3=0, 1 or 2; and n4=1 or 2.

In another preferred embodiment, Cr¹ is selected from the group consisting of:

Null,

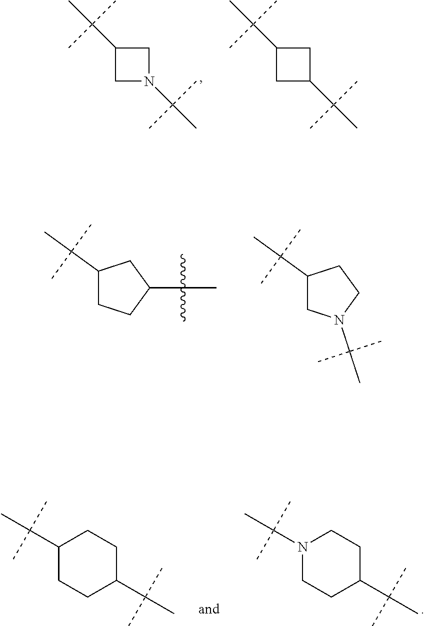

In another preferred embodiment, Cr² is selected from the group consisting of:

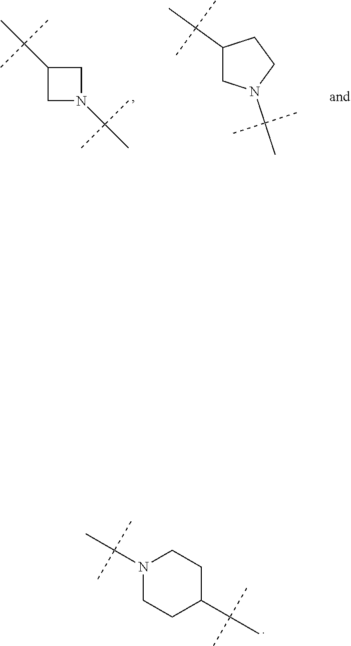

In a specific embodiment the resent invention provides a conjugate as shown below;

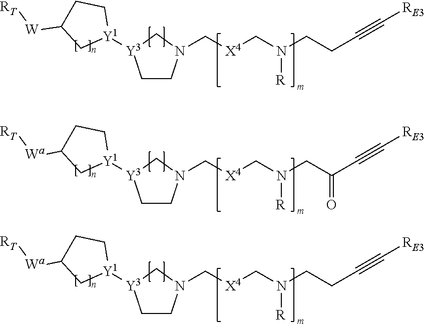

wherein
X⁴ is selected from the group consisting of CH₂, O, NH, and NR;
Y¹ and Y³ are each independently selected from the group consisting of CH, and N;
W^{a} is selected from the group consisting of NH, and O;
m=0, 1, 2, 3 etc. (preferably, m is not 0);
n=0, 1, 2, 3, etc. (preferably, n is not 0);
R_{T}, R_{E3} and R are defined as above; preferably, R is H, C1-6alkyl (such as Me, Et etc.), Ac, CHO, and CONH₂.

In another specific embodiment, the conjugate is a conjugate selected from Group 2:

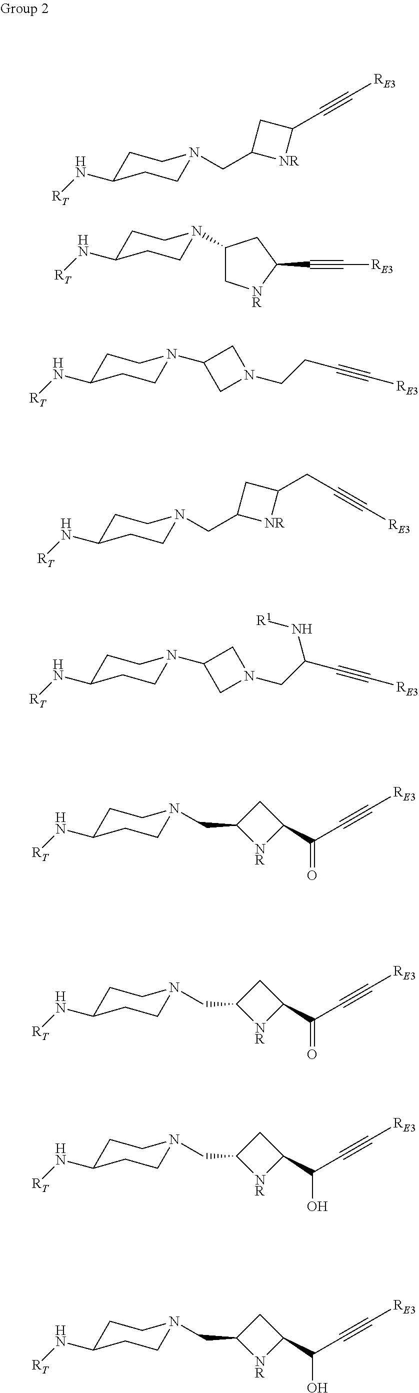

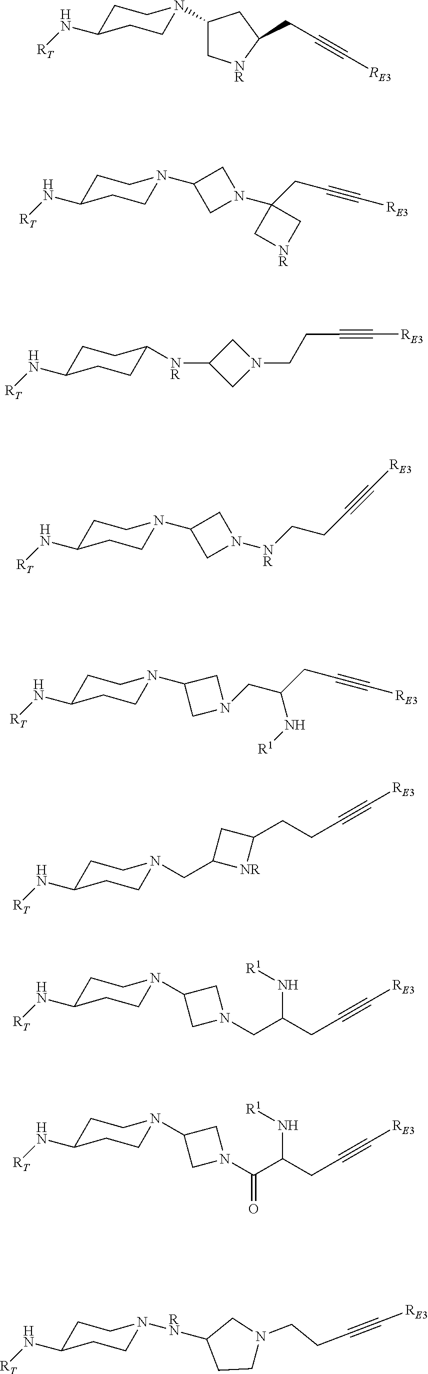

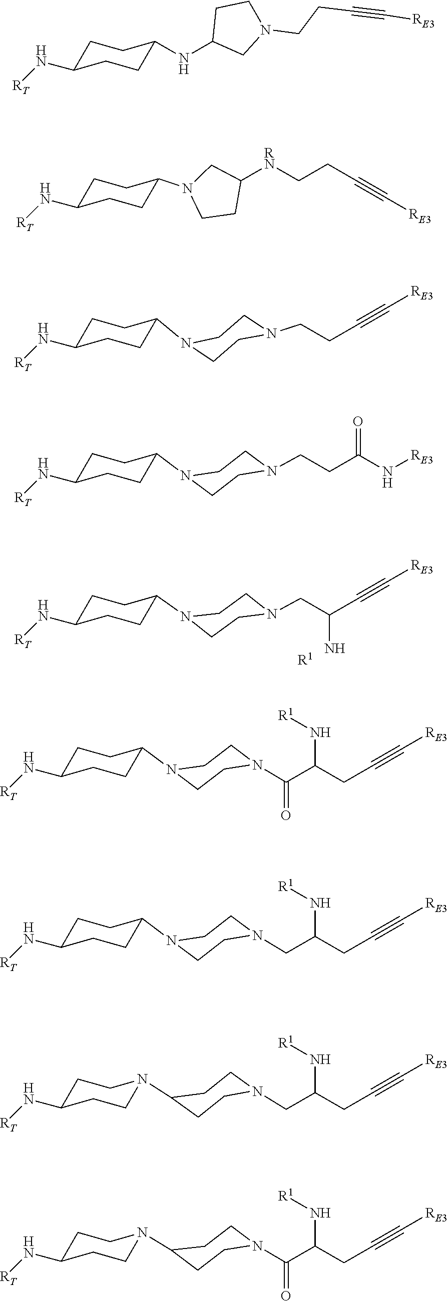

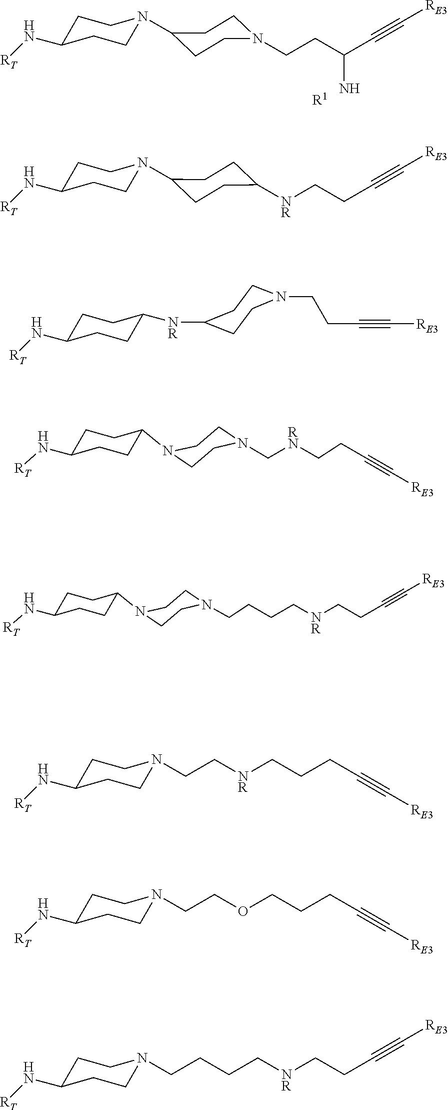

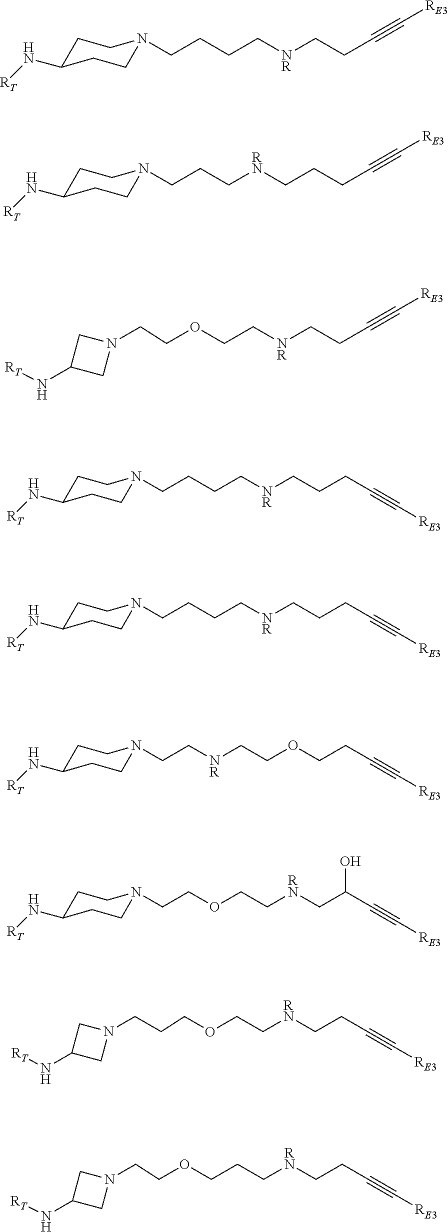

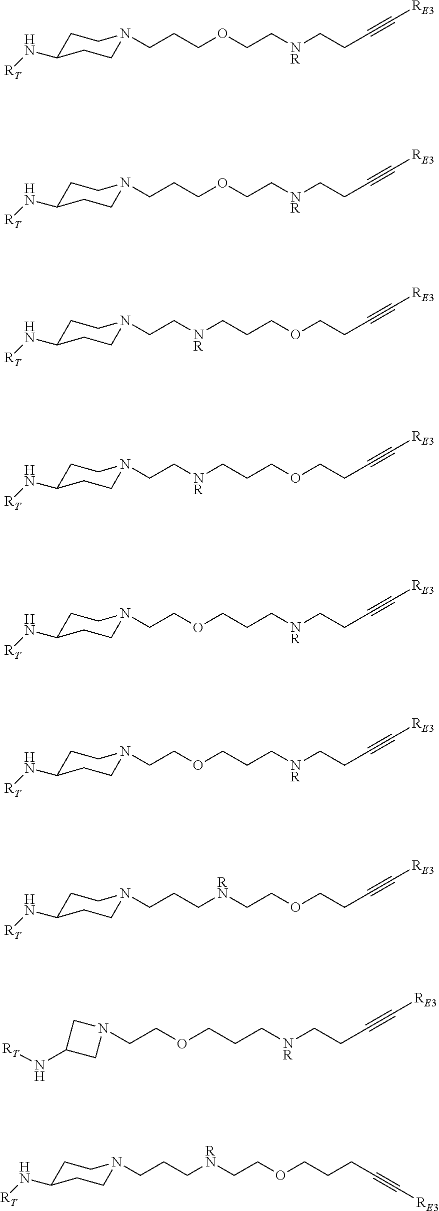

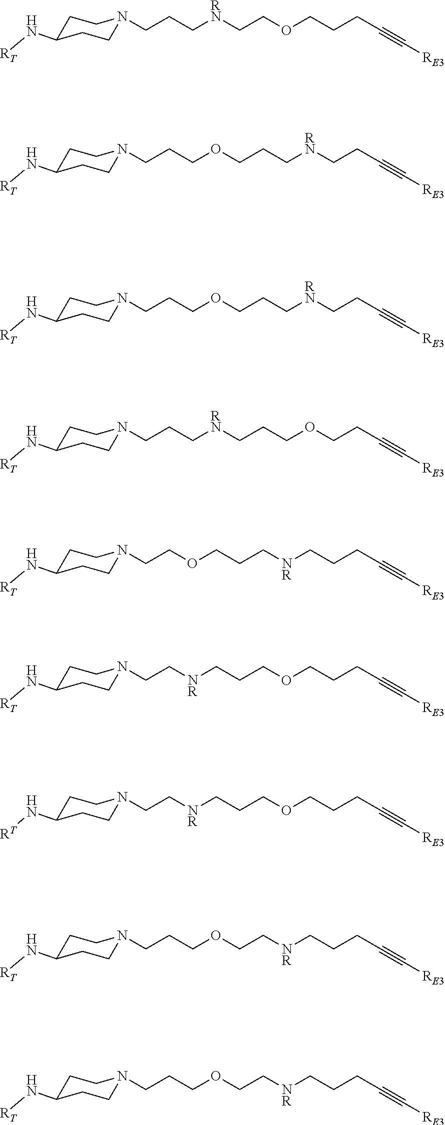

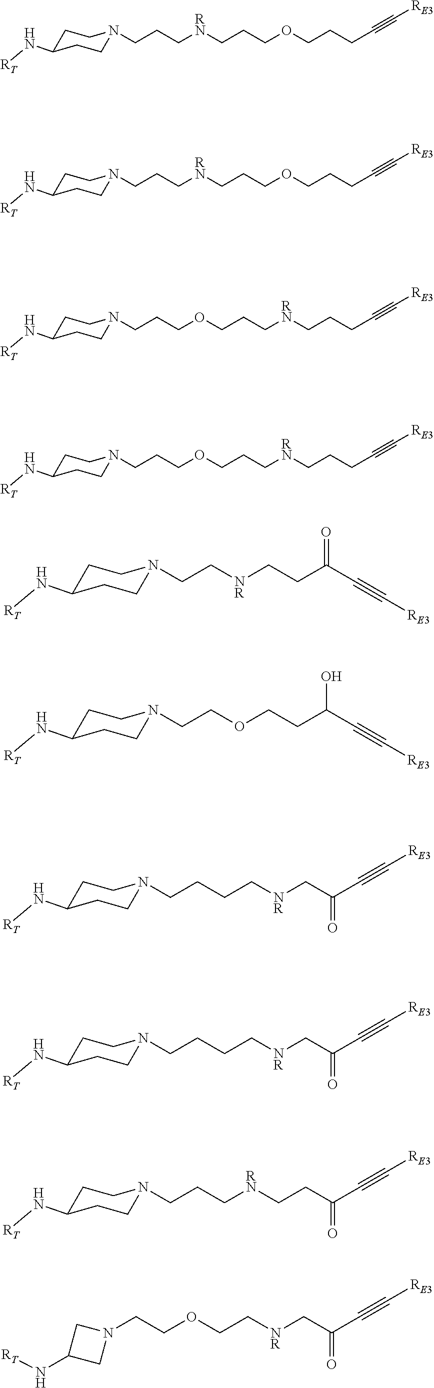

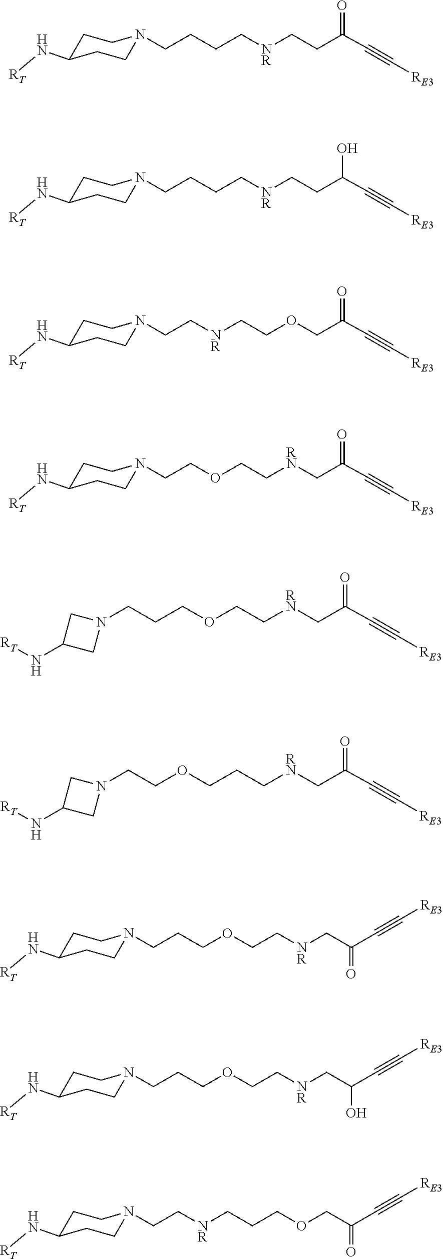

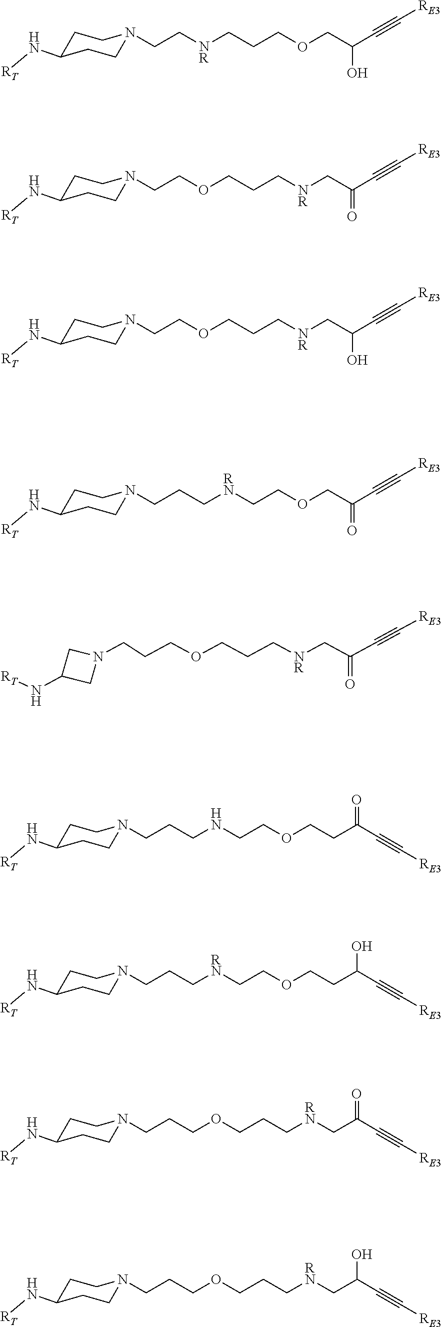

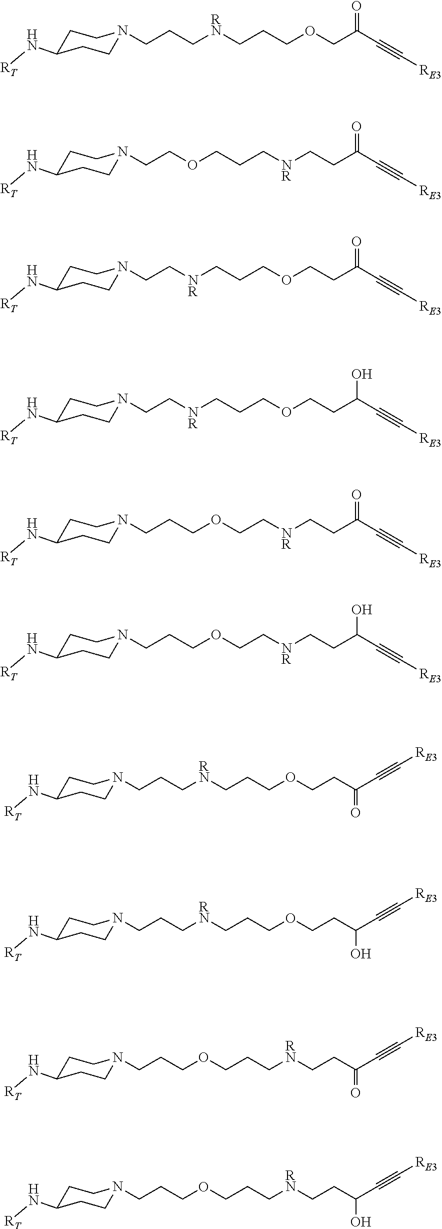

wherein R_{T}, R_{E3}, R and R₁ are defined as above; preferably, R and R¹ are each independently —W³-L3-W⁴—(R_{P})_{q}, wherein W³, L3, W⁴, R_{P} and m are defined as above.

In a specific embodiment, the present invention further provides a conjugate as shown in R_{T}—W^{a}—Cr¹—W^{a}—Cr²-L6-W²—R_{E3} (I-2a);
wherein
W^{a} is defined as W;
Cr¹ is null, or C₄₋₇cycloalkyl that is unsubstituted or substituted with C₁₋₄alkyl, or 4 to 6 membered heterocyclyl that is unsubstituted or substituted with C₁₋₄alkyl;
Cr² is 4 to 6 membered heterocyclyl containing nitrogen that is unsubstituted or substituted with C₁₋₄alkyl, and at least one nitrogen heteroatom in Cr² is attached with L5;
W, R_{T}, R_{E3}, W² and L5 are defined as above.

In another preferred embodiment, W² is selected from the group consisting of: W^{b}—C≡C, C(O), and C(O)NH.

In a specific embodiment, the present invention further provides a conjugate as shown in R_{T}—W^{a}—Cr¹—Cr²-L6-W^{b}—C≡C—R_{E3}; wherein W^{a}, W^{b}, Cr¹, Cr², R_{T}, R_{E3} and L5 are defined as above.

Preferably, the conjugate is selected from the group consisting of:
R_{T}—NH—Cr¹—Cr²-L6-CH₂—C≡C—R_{E3};
R_{T}—NH—Cr¹—Cr²-L6-C(O)—C≡C—R_{E3};
R_{T}—NH—Cr¹—Cr²-L6-CH(OH)—C≡C—R_{E3};
and R_{T}—NH—Cr¹—Cr²-L6-C≡C—R_{E3};
in each formula, R_{T}, R_{E3}, Cr¹, Cr² and L6 are defined as above.

In another specific embodiment, the conjugate is a conjugate selected from Group 2a:

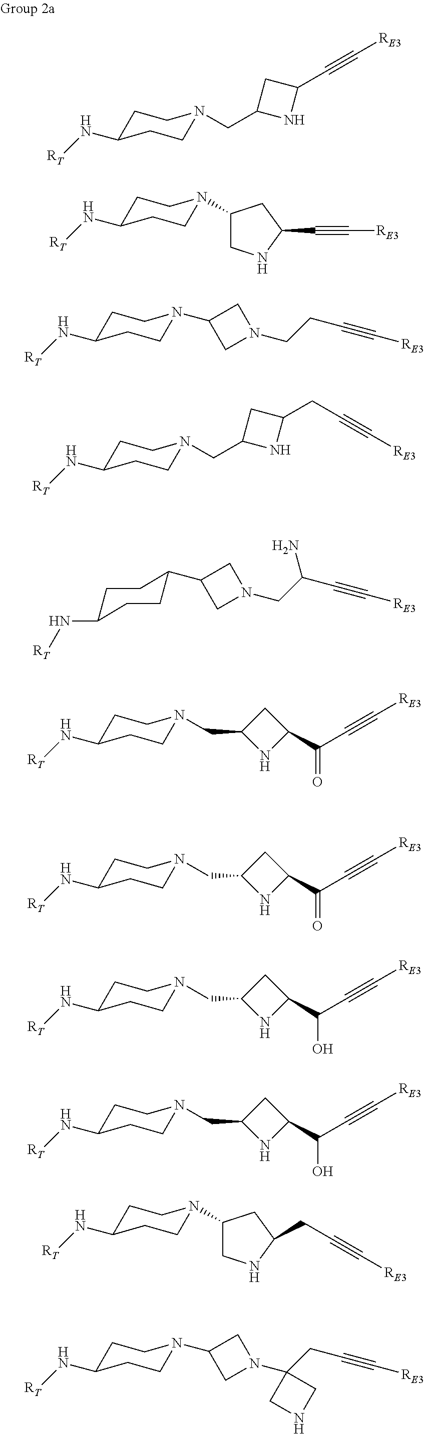

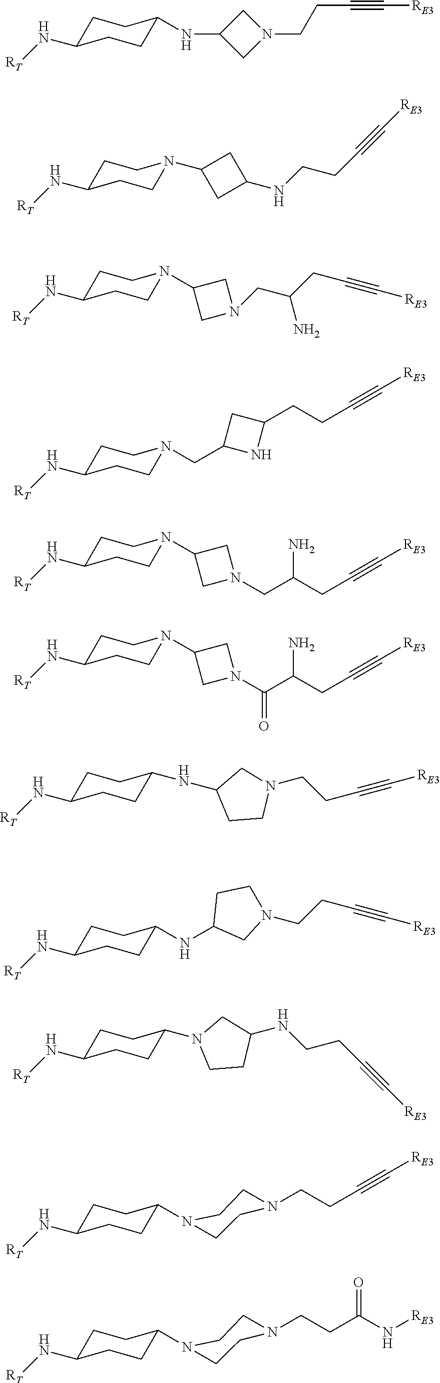

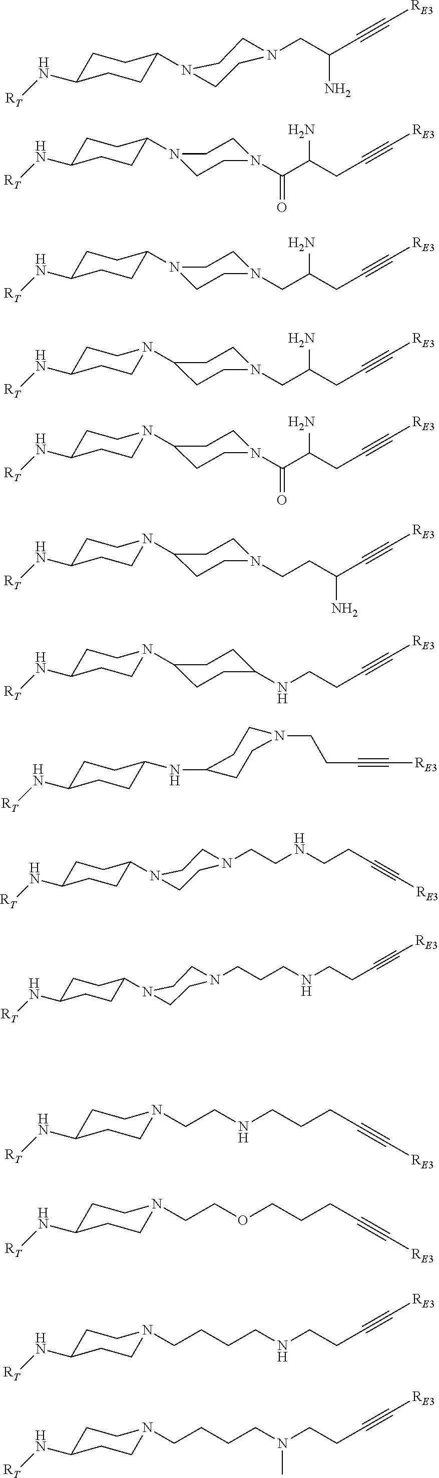

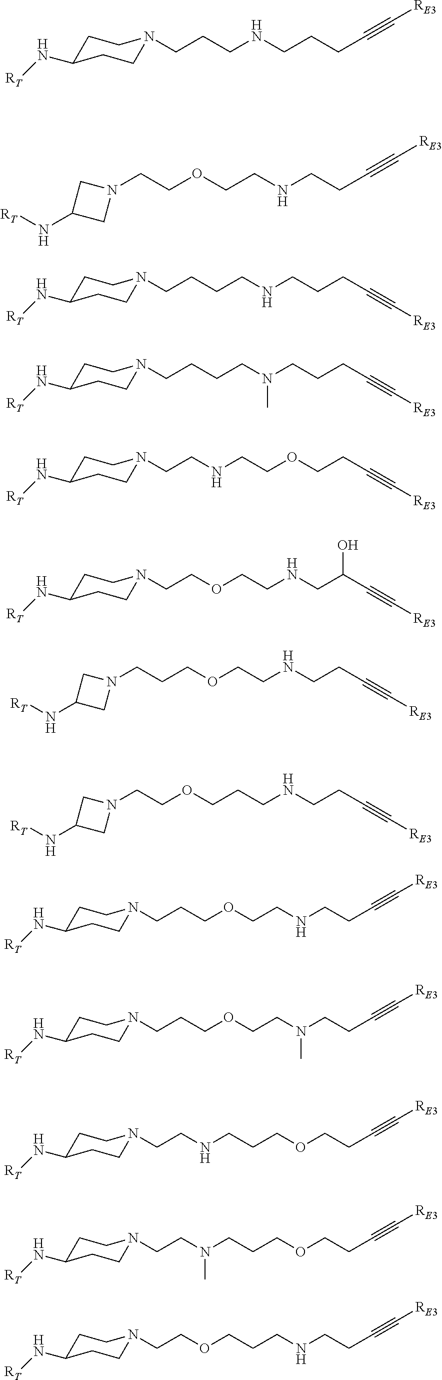

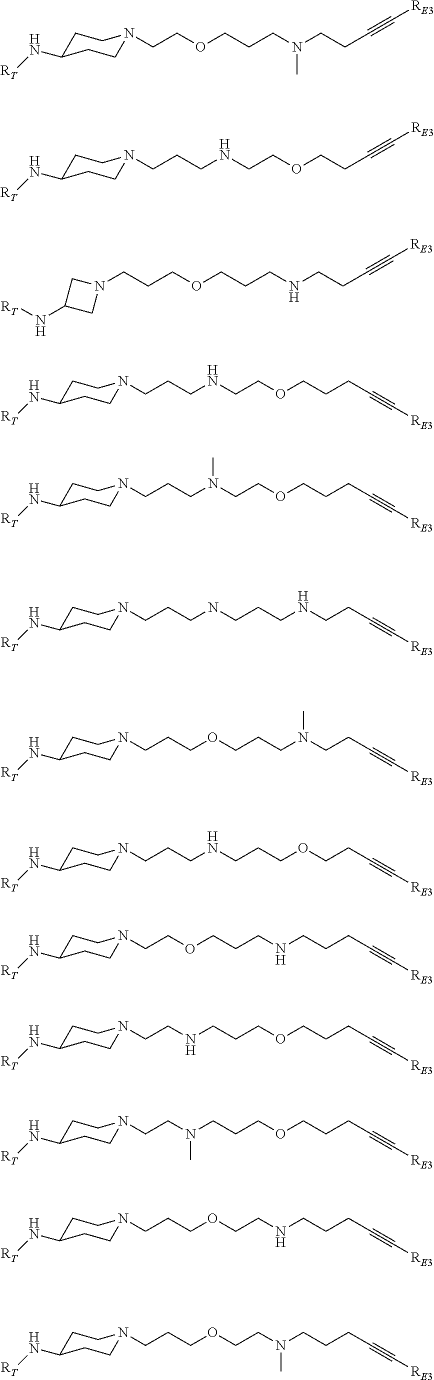

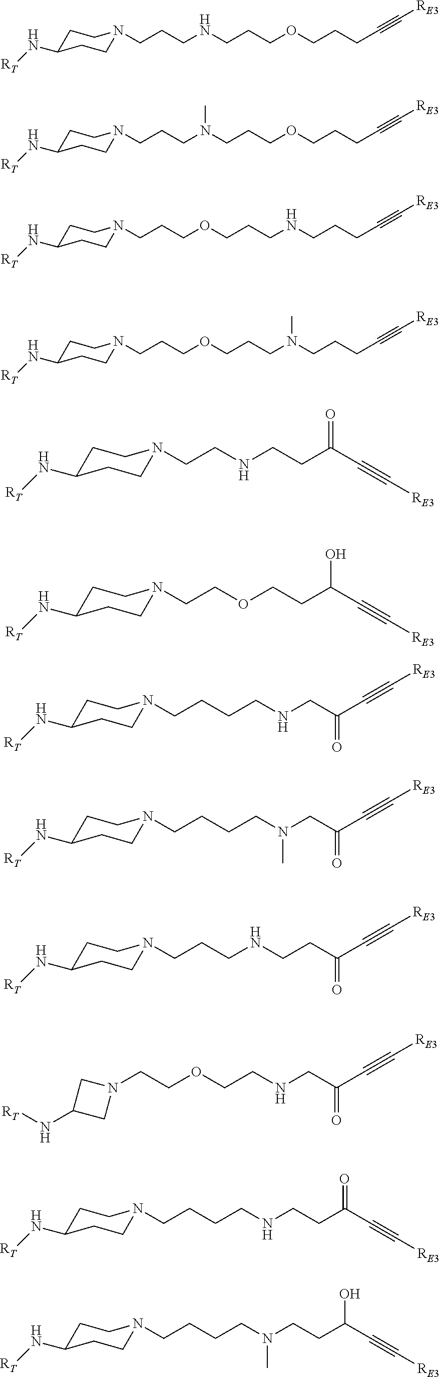

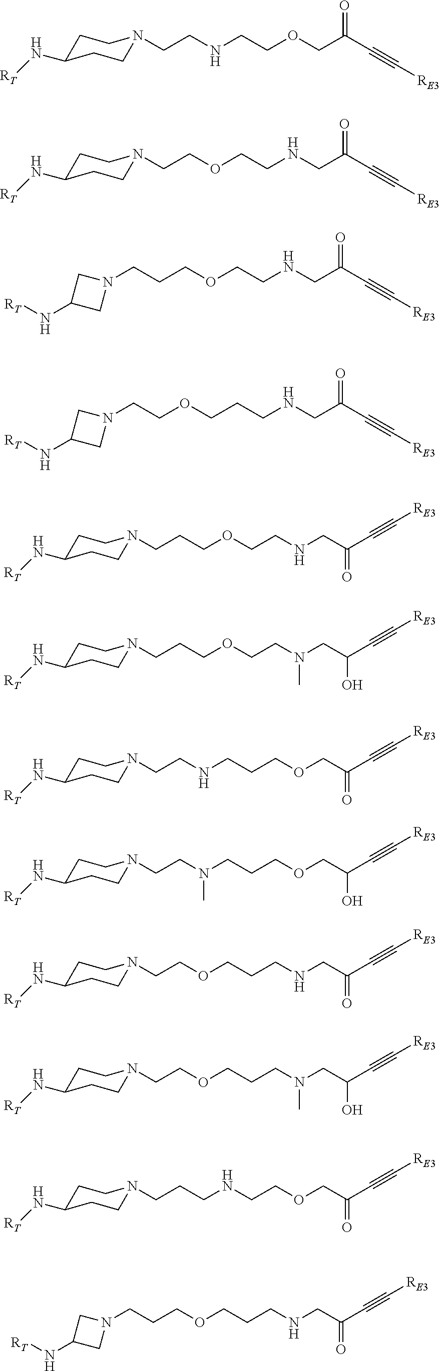

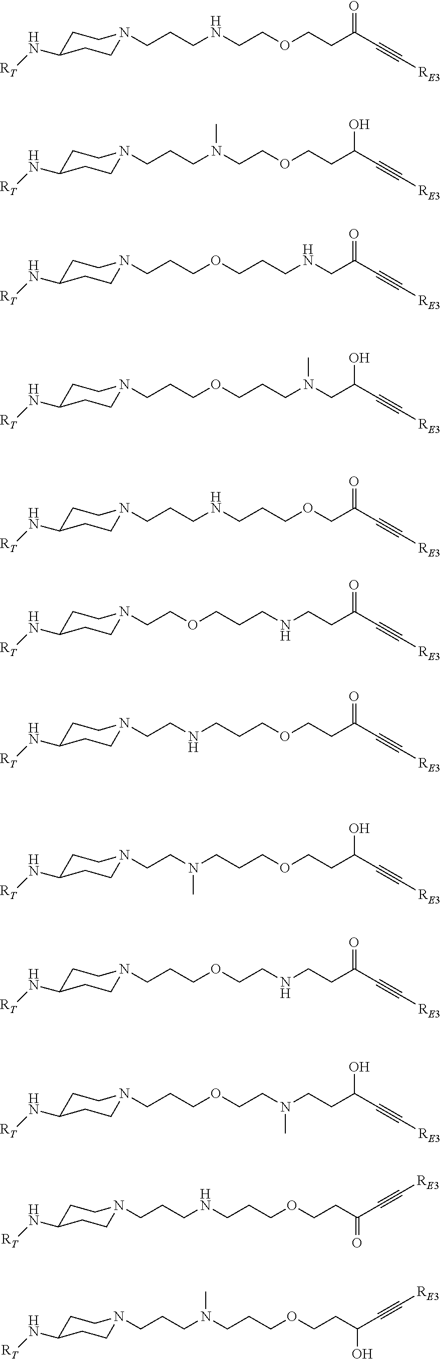

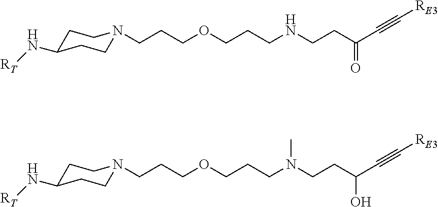

In a specific embodiment, the present invention provides a conjugate of R_{T}-Ar1-L5-W^{Z}—R_{E} (3);
wherein Ar1 is −5 or 6 membered heteroaryl containing nitrogen atom-; L5, R_{T}, W2 and R_{E3} are defined as above.

In another preferred embodiment, W2 is selected from —CONH—, —CO—, —CONH—, and —W^{b}—C≡C—.

In a specific embodiment, the present invention provides a conjugate of R_{T}-Ar1-L5-CONH—R_{E3}, R_{T}-Ar1-L5-CO—R_{E3} or R_{T}-Ar1-C≡C—R_{E3};
wherein Ar1 is −5 or 6 membered heteroaryl containing nitrogen atom-; L5, R_{T}, and R_{E3} are defined as above.

In another preferred embodiment, Ar1 is

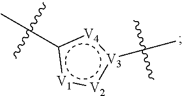

wherein V₁, V₂ and V₄ are each independently selected from —O—, —S—, —N═, —NH—, —CH═, —CH₂—; V₃ is selected from the group consisting of —N═, —CH═.

In a specific embodiment, the present invention provides a conjugate as shown below;

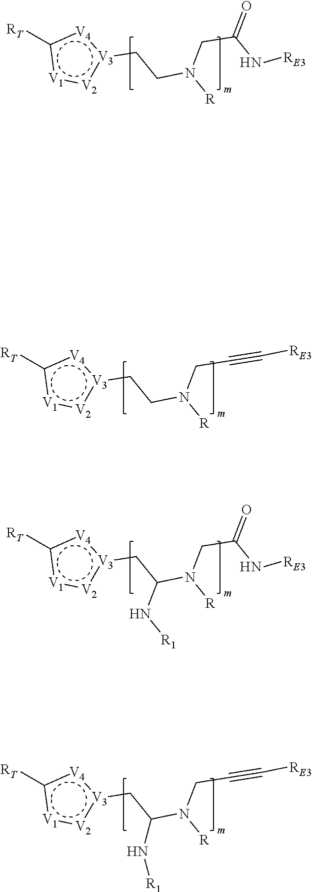

In each formula,
V₁, V₂ and V₄ are each independently selecte from —O—, —S—, —N═, —NH—, —CH═, —CH₂;
V₃ is selected from the group consisting of —N═, —CH═;
R, R₁, R_{T} and R_{E3} are defined as above;
m=0, 1, 2, 3, 4 and other integers (preferably m is not 0).

In a specific embodiment, the present invention provides a conjugate as shown below;

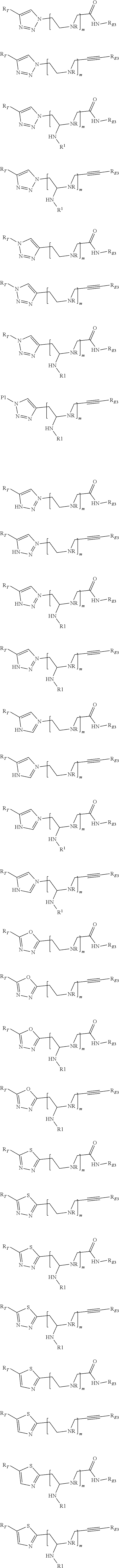

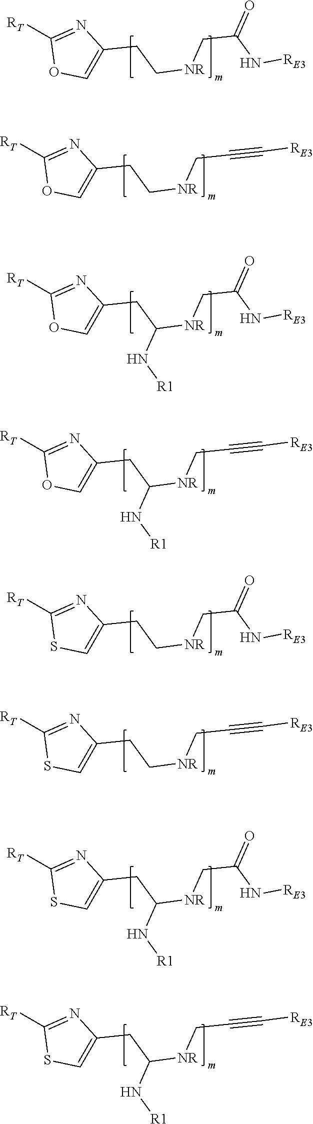

In each formula,
R, R₁, R_{T} and R_{E3} are defined as above;
m=0, 1, 2, 3, 4 and other integers (preferably m is not 0).

In another specific embodiment, the conjugate is selected from Group 3:

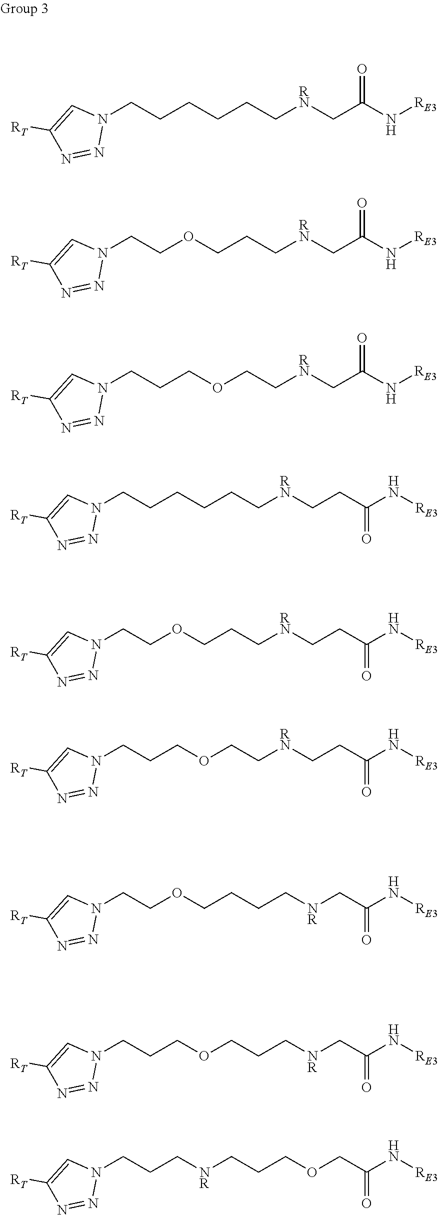

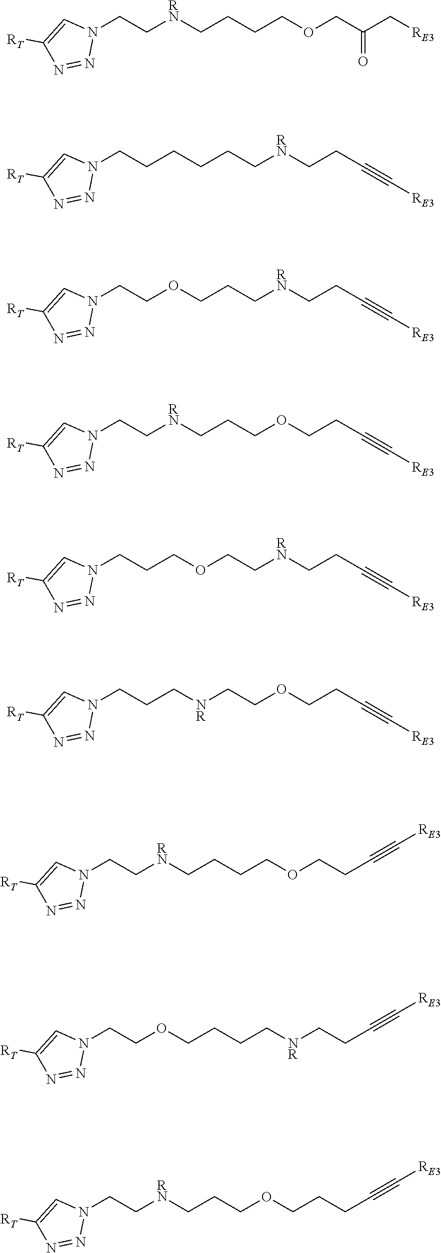

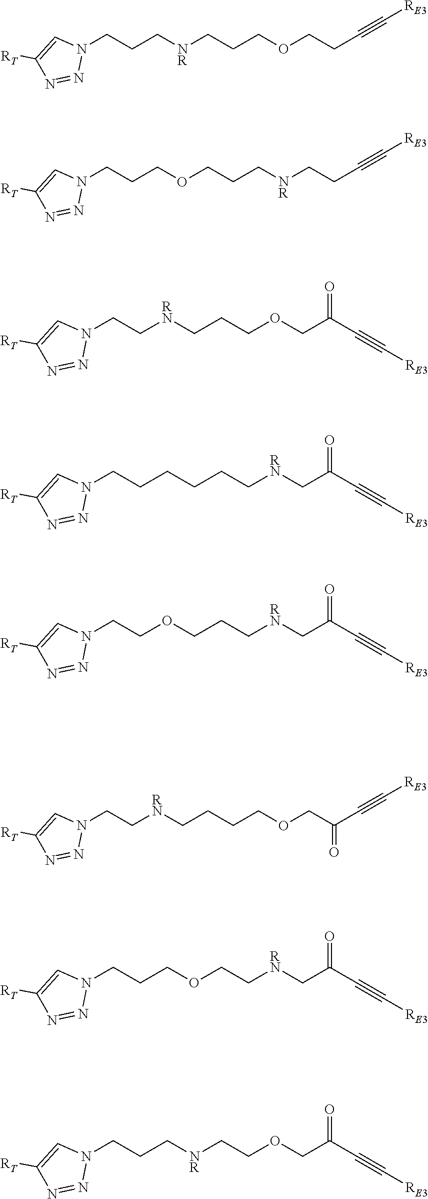

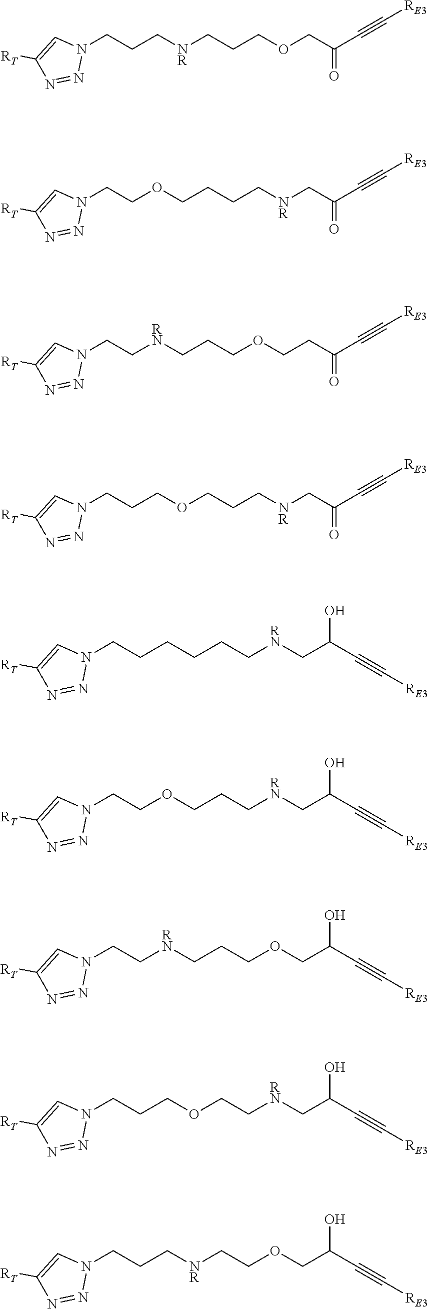

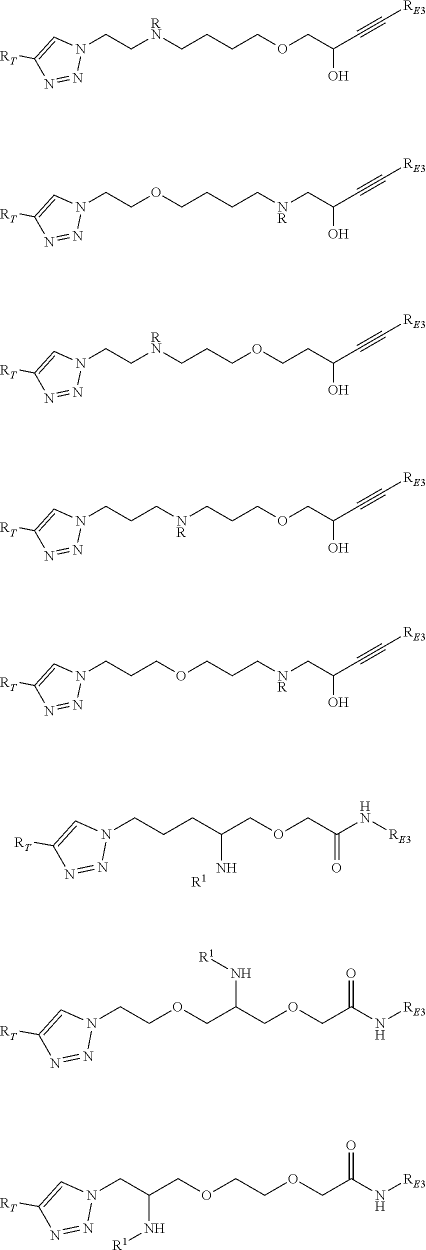

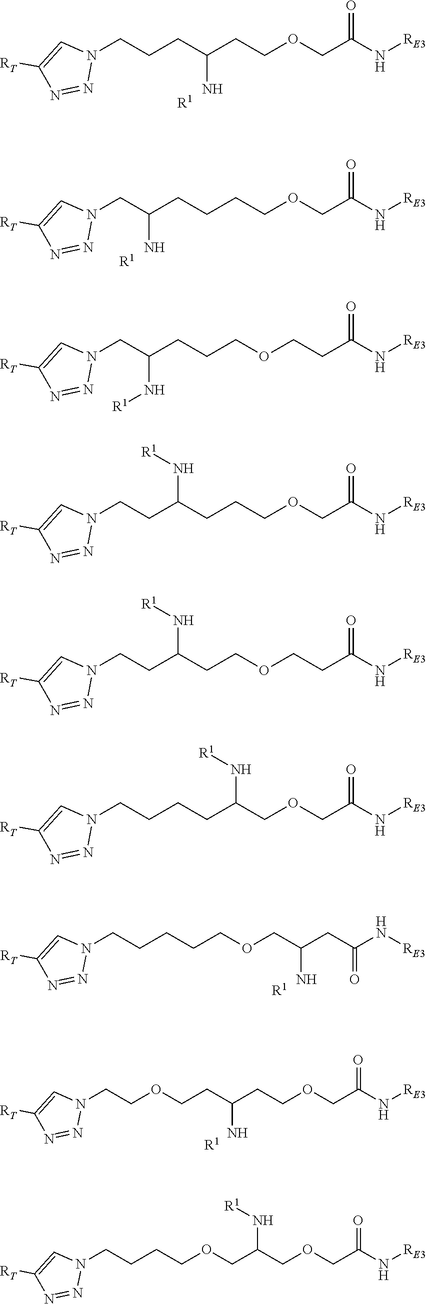

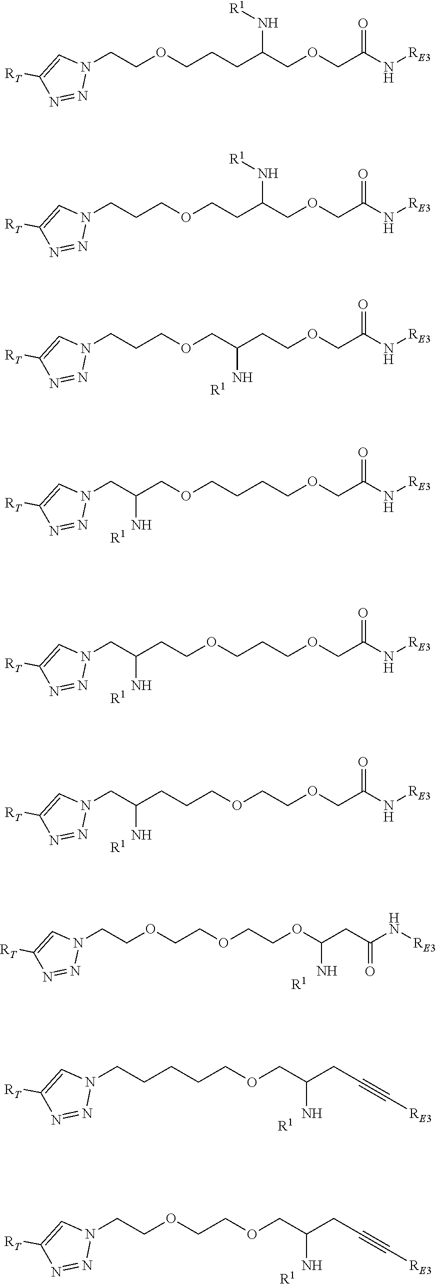

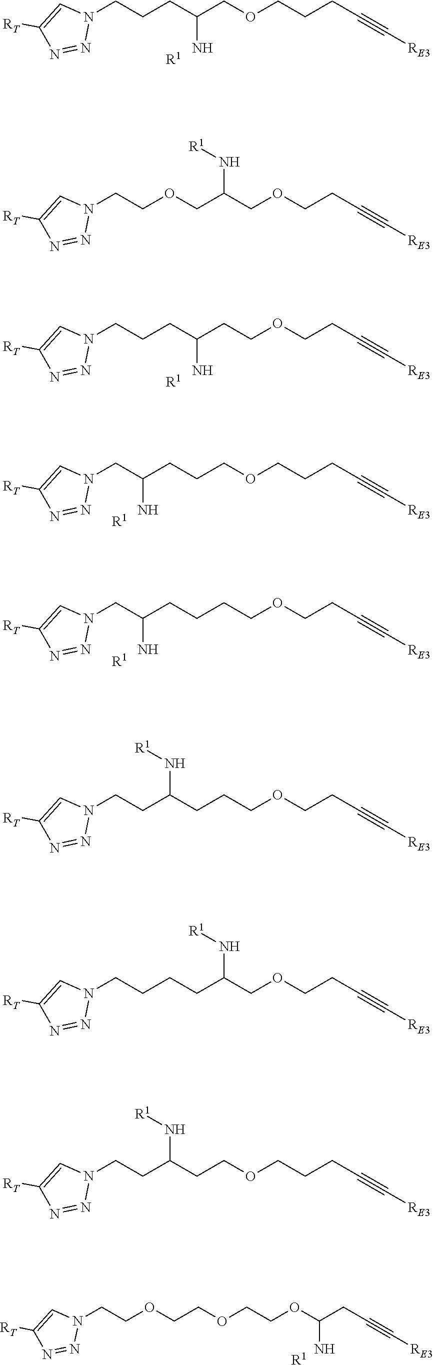

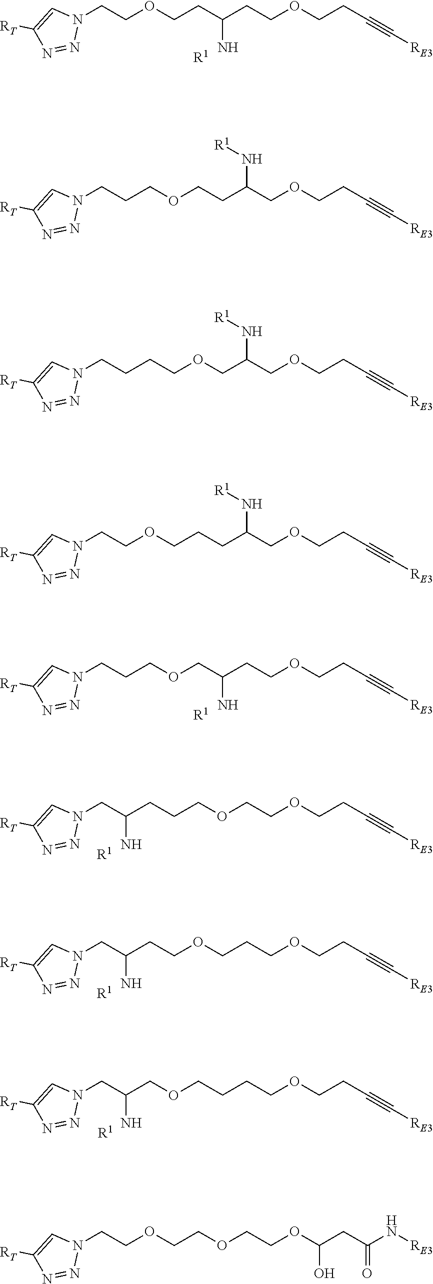

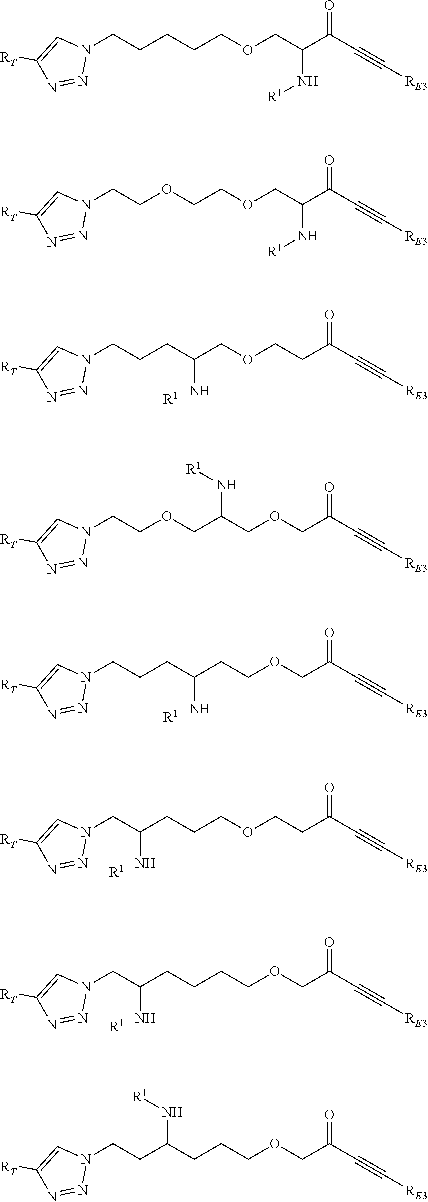

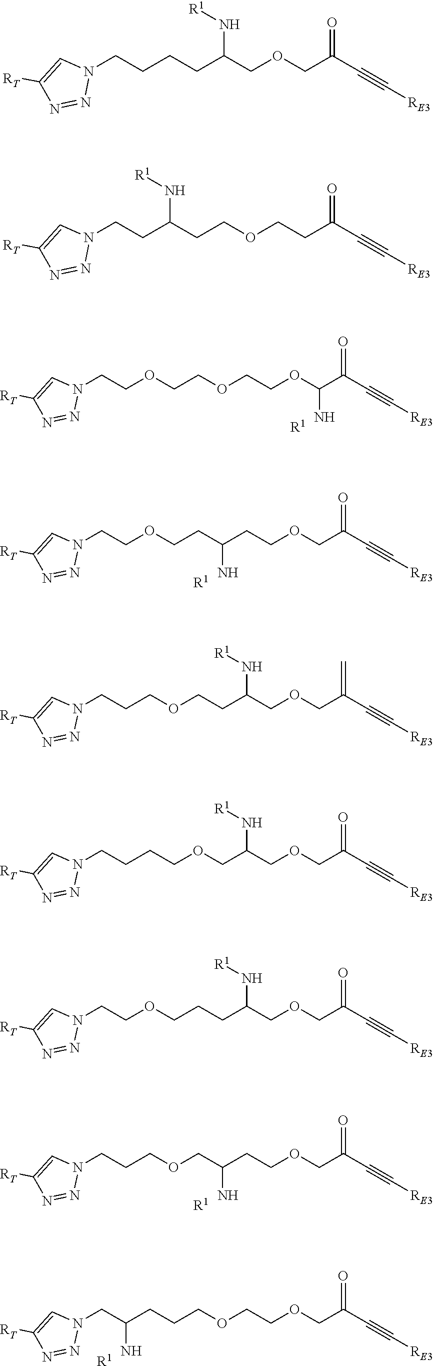

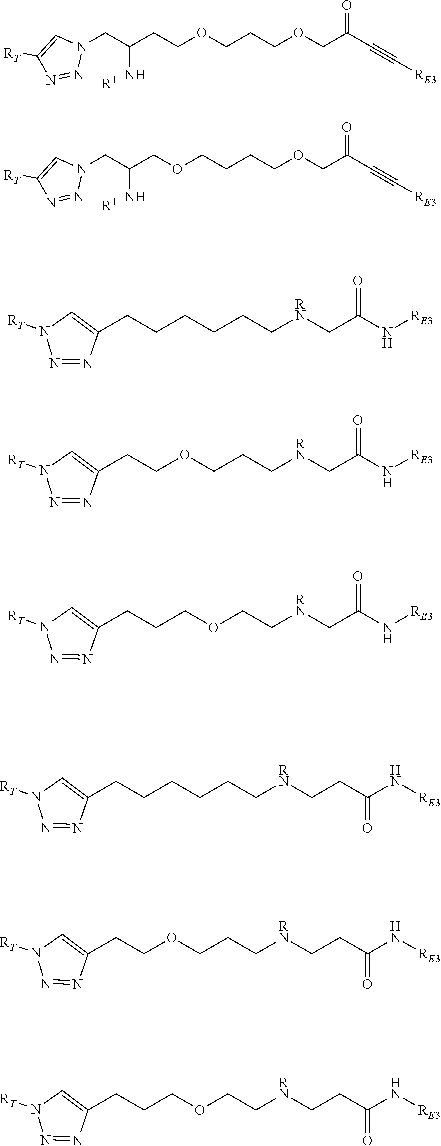

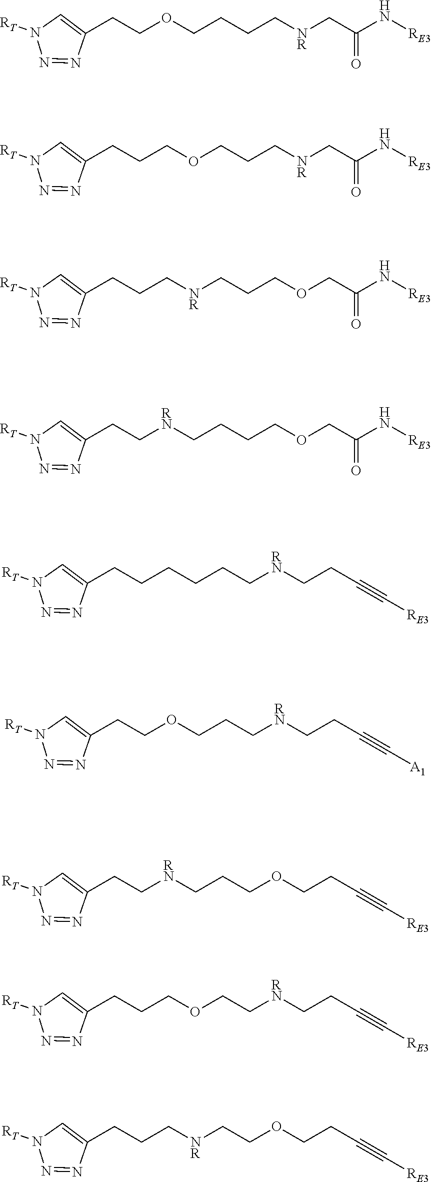

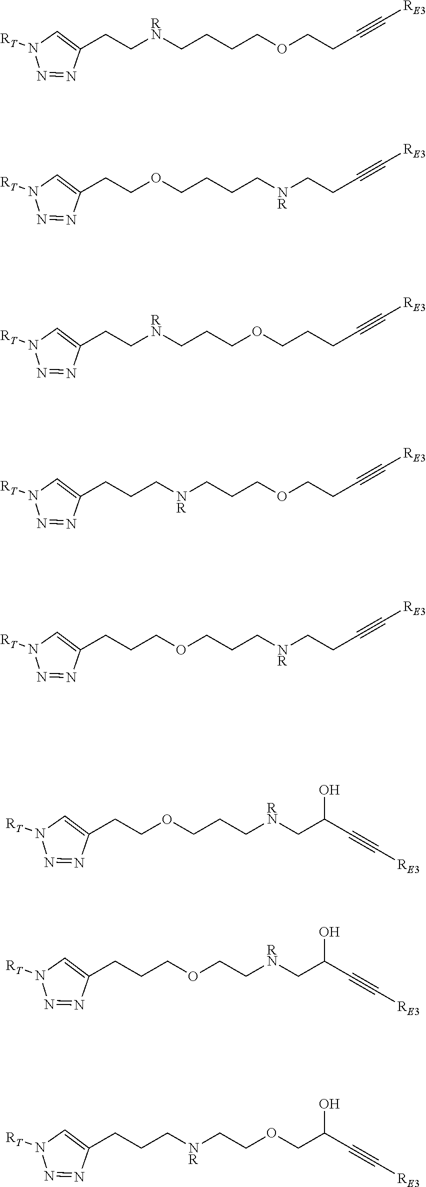

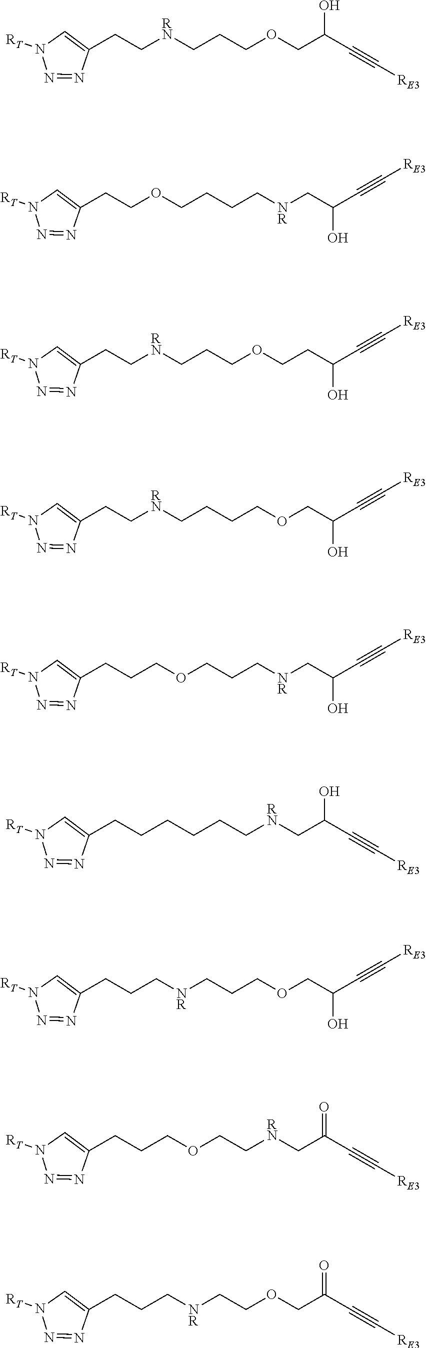

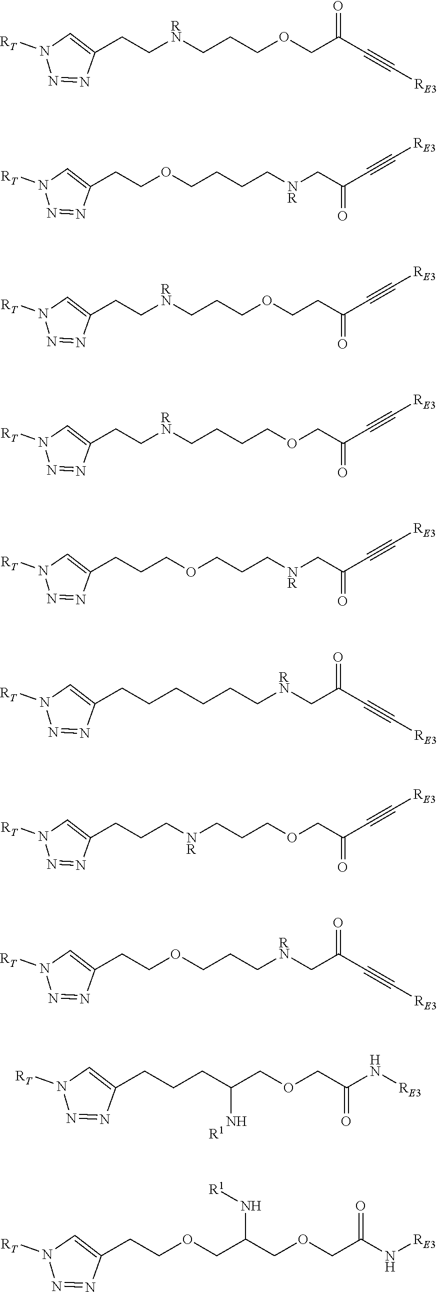

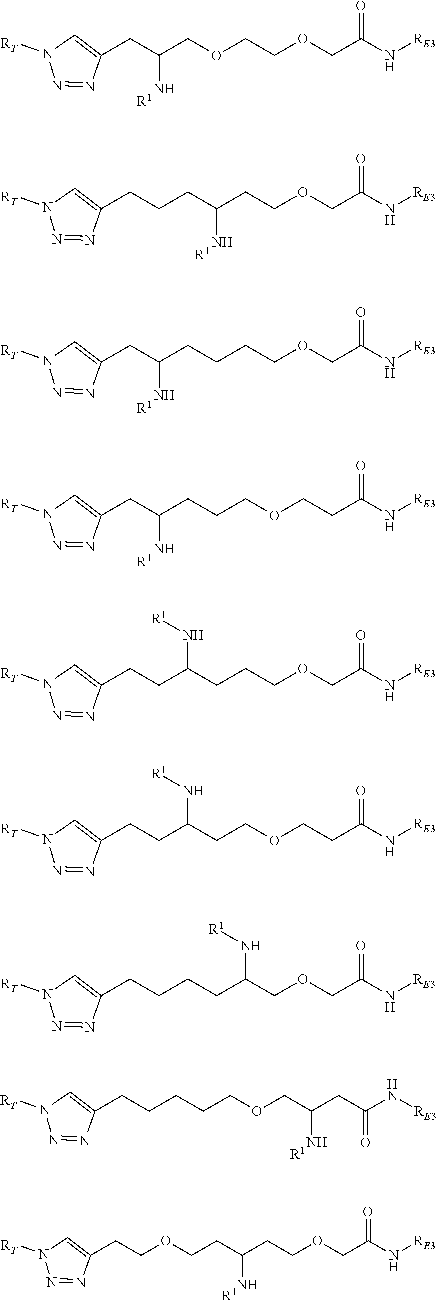

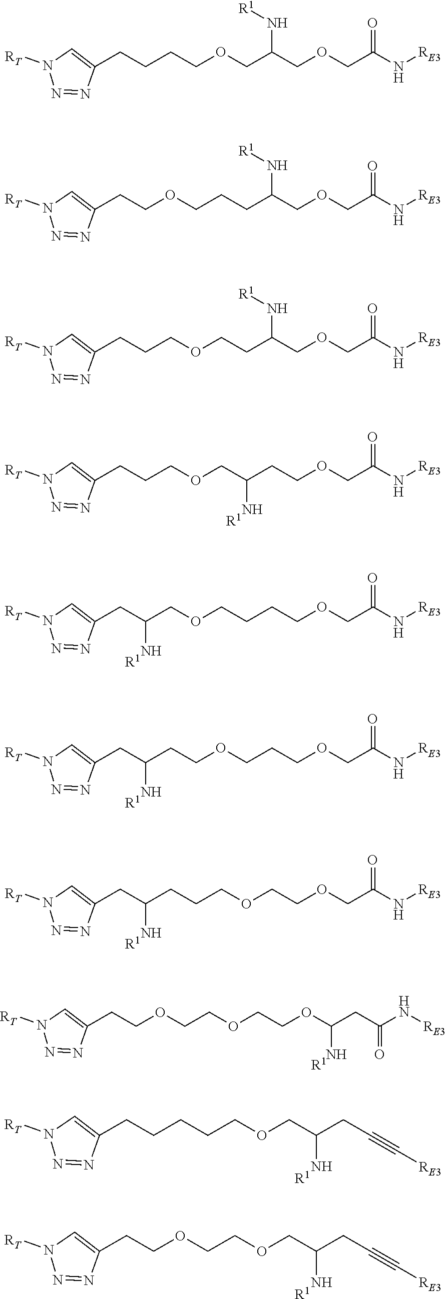

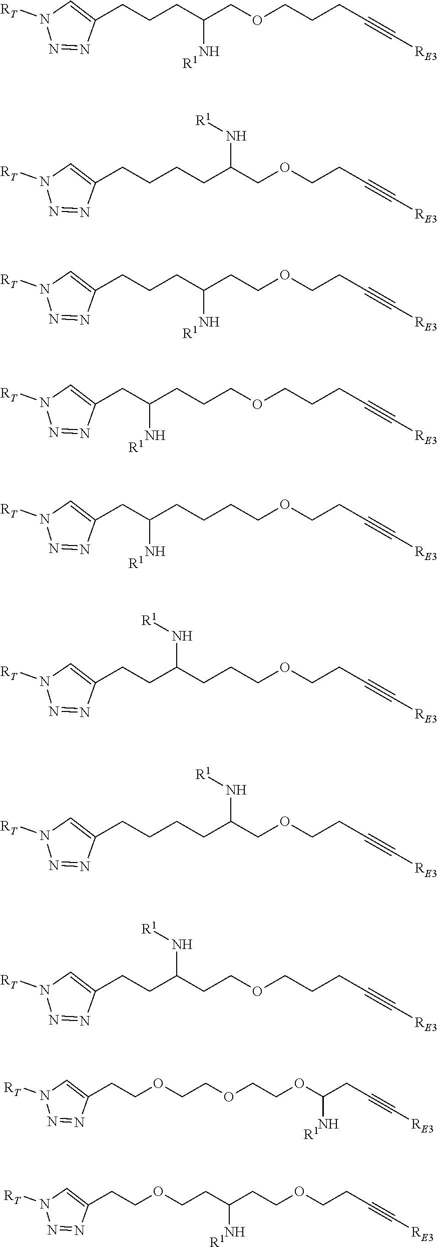

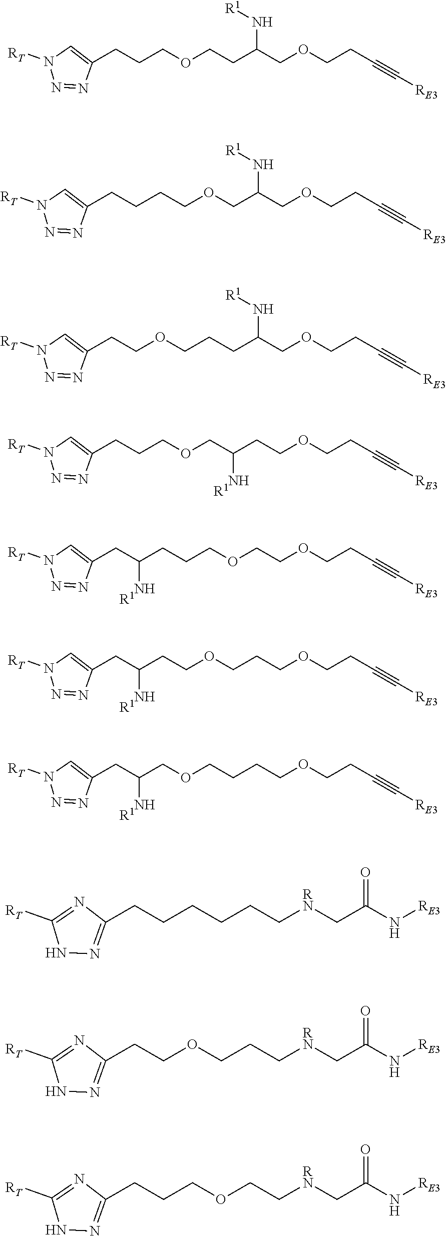

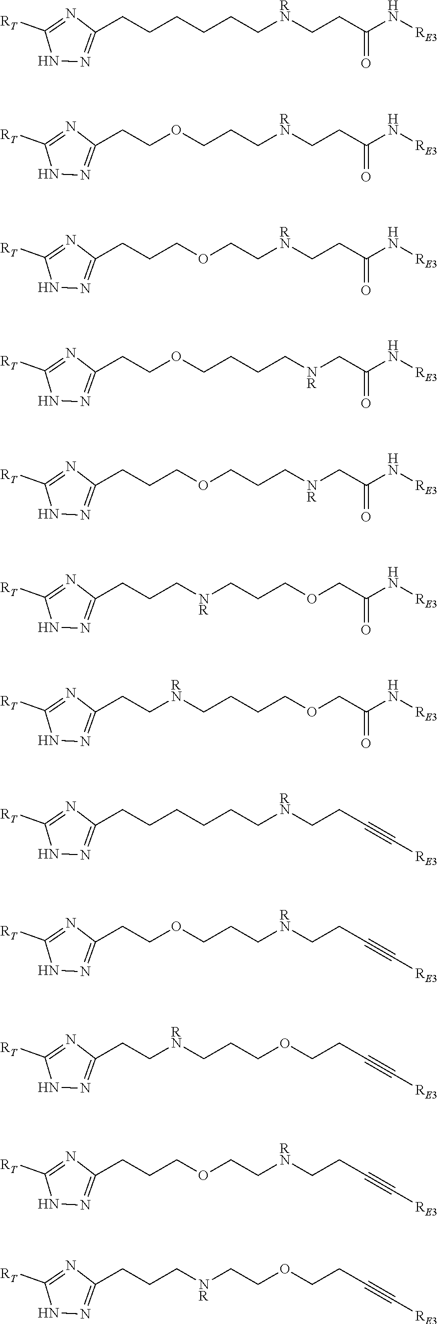

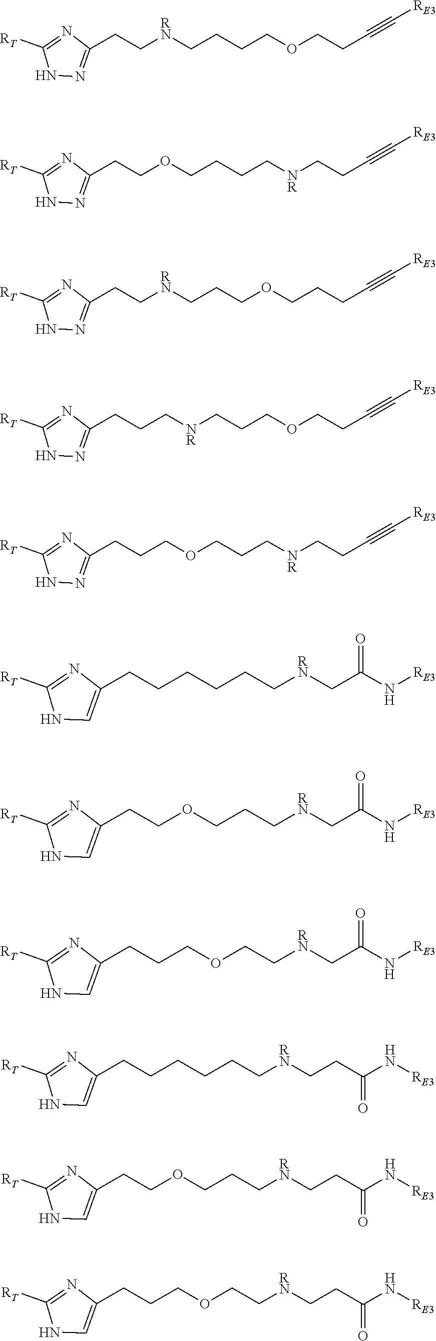

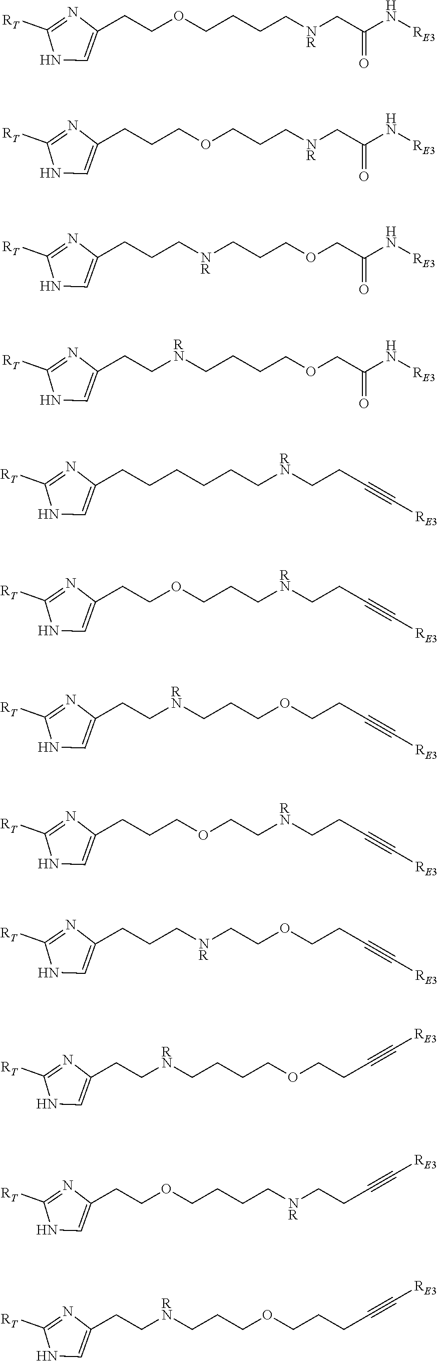

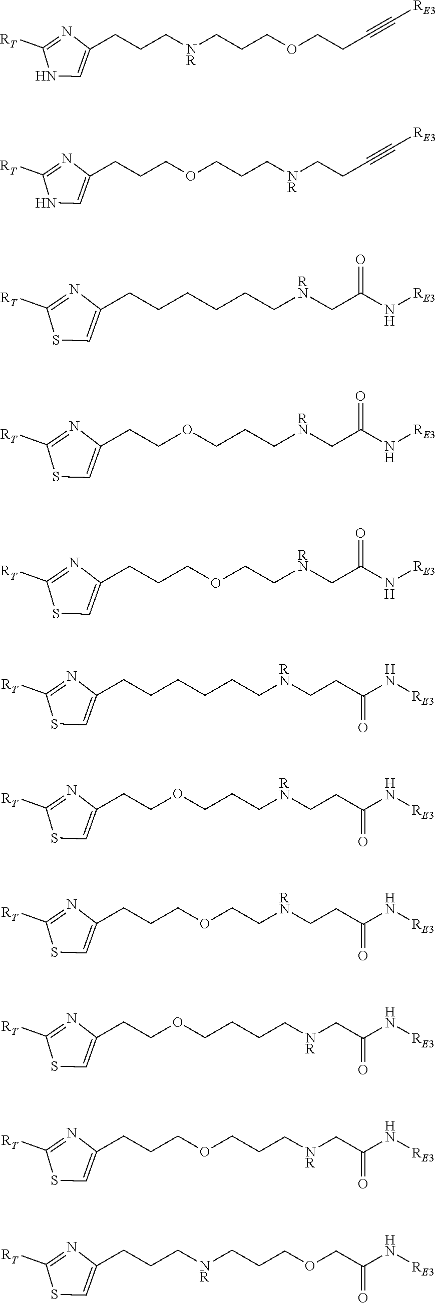

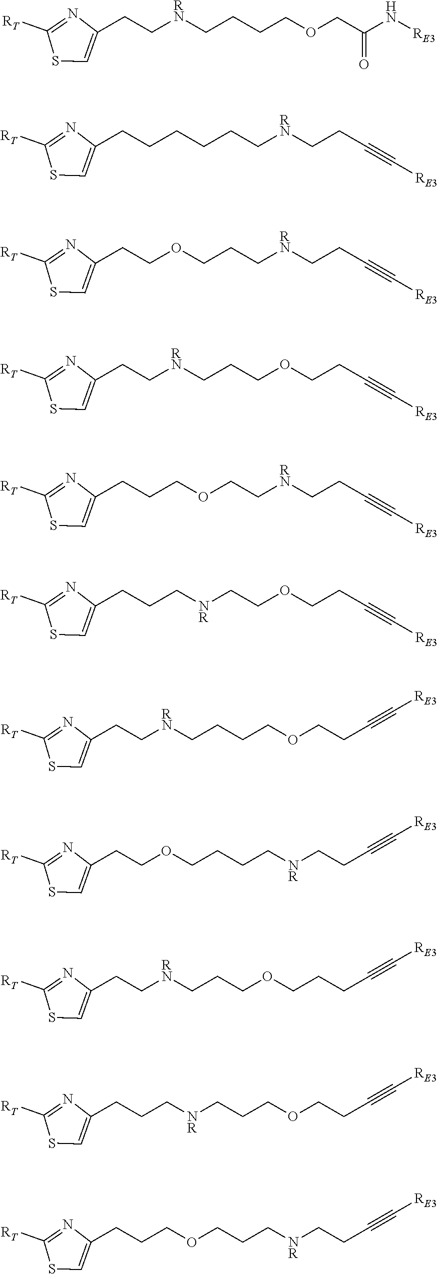

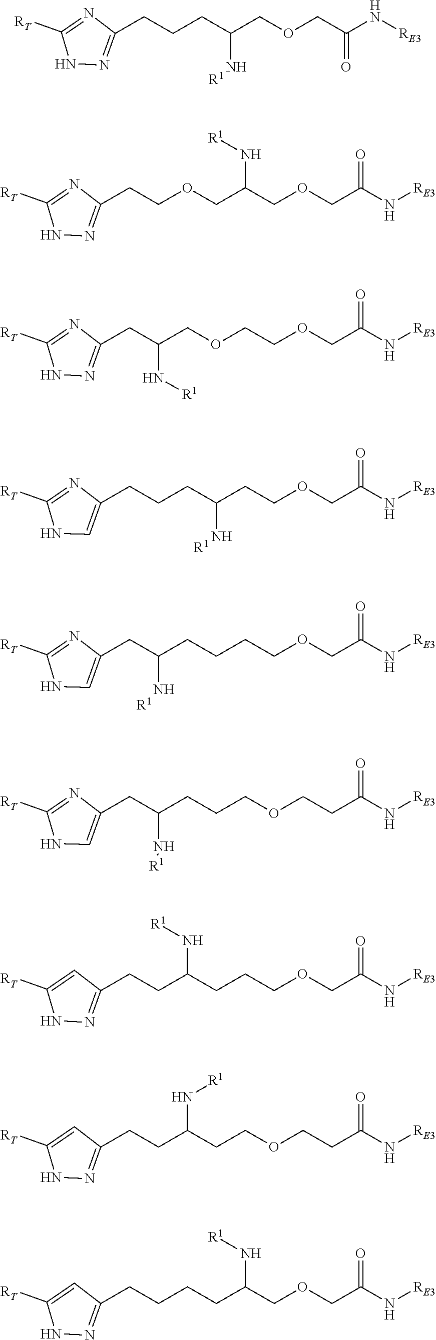

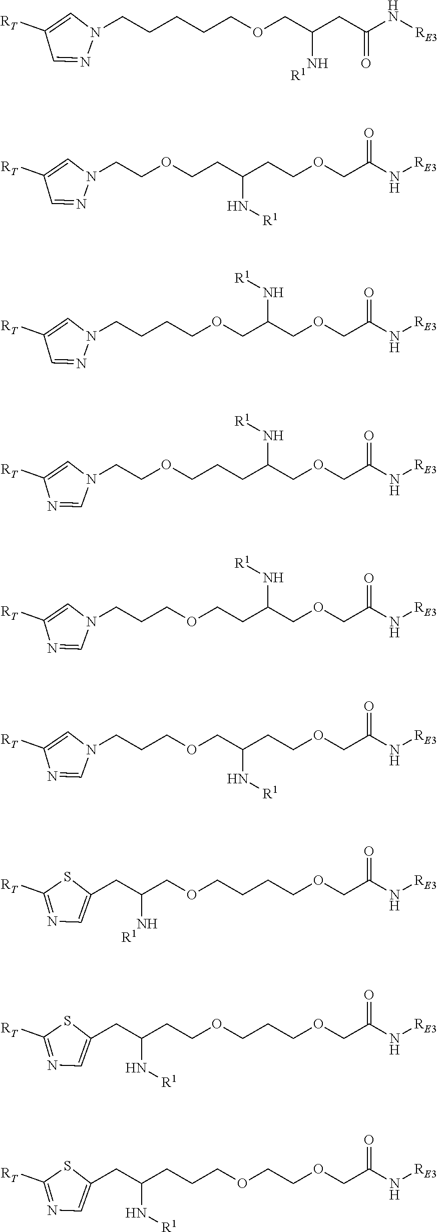

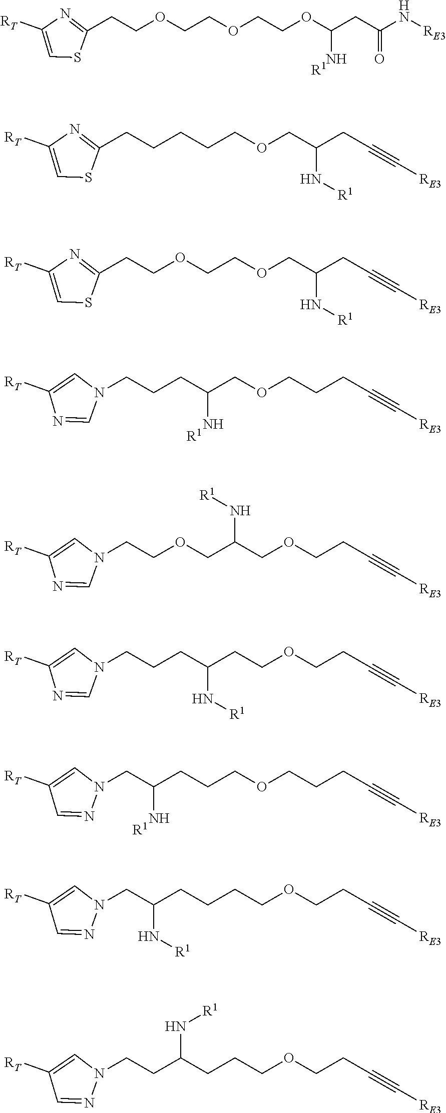

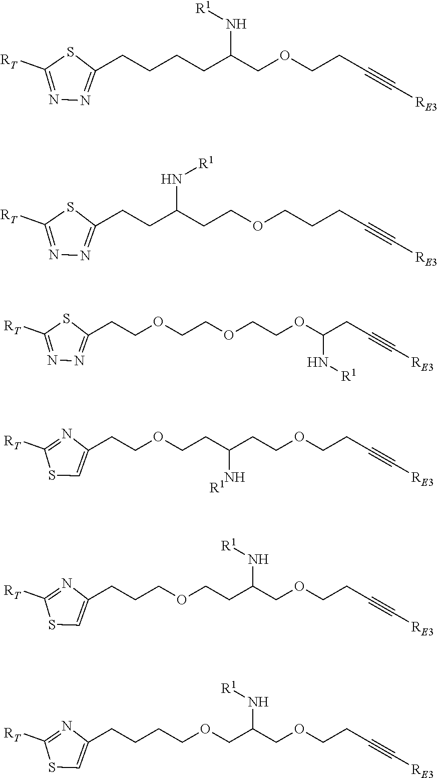

wherein R_{T}, R_{E3}, R and R₁ are defined as above; preferably, R and R are each independently —W³-L3-W⁴—(R_{P})_{q}, wherein W³, L3, W⁴, R_{P} and m are defined as above.

In a specific embodiment, the present invention further provides a conjugate of R_{T}-Ar1-L6-W2-R_{E};
wherein Ar¹, L5, R_{T}, W² and R_{E3} are defined as above.

In a specific embodiment, the present invention provides a conjugate of R_{T}—Ar¹-L6-CONH—R_{E3}, R_{T}—Ar¹-L6-CO—R_{E3} or R_{T}—Ar¹-L6-W^{b}—C≡C—R_{E3}; wherein Ar¹, L6, R_{T} and R_{E3} are as defined as above.

In another specific embodiment, the conjugate is selected from Group 3a-1˜Group 3a-5:

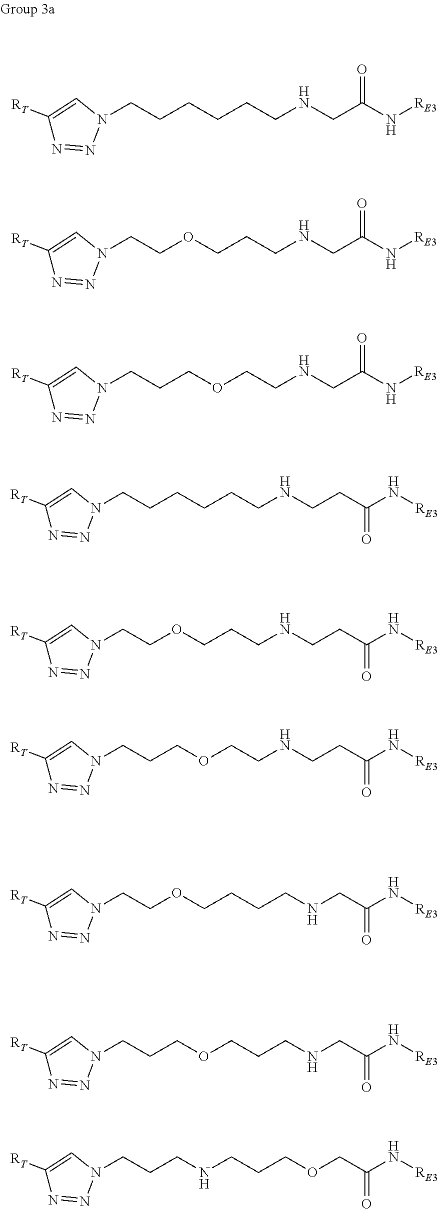

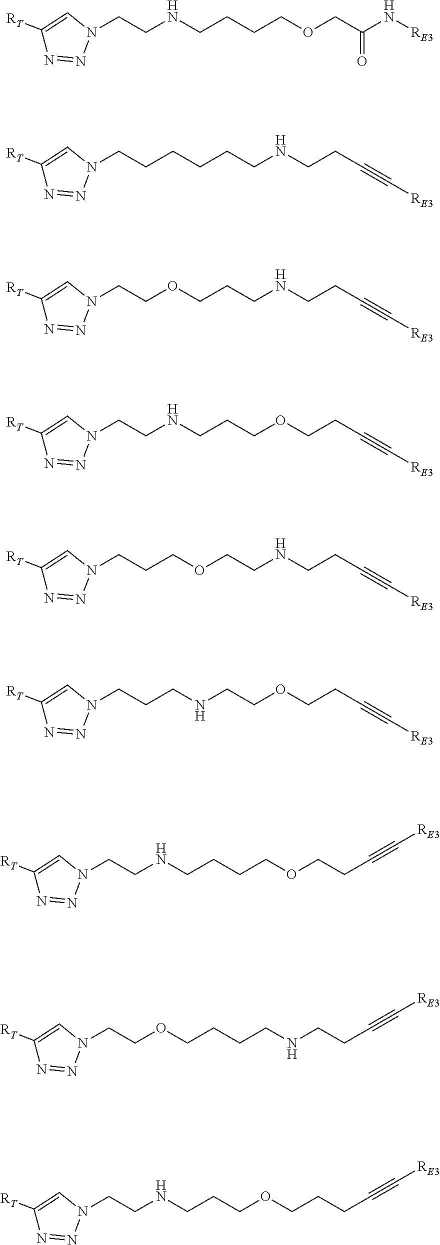

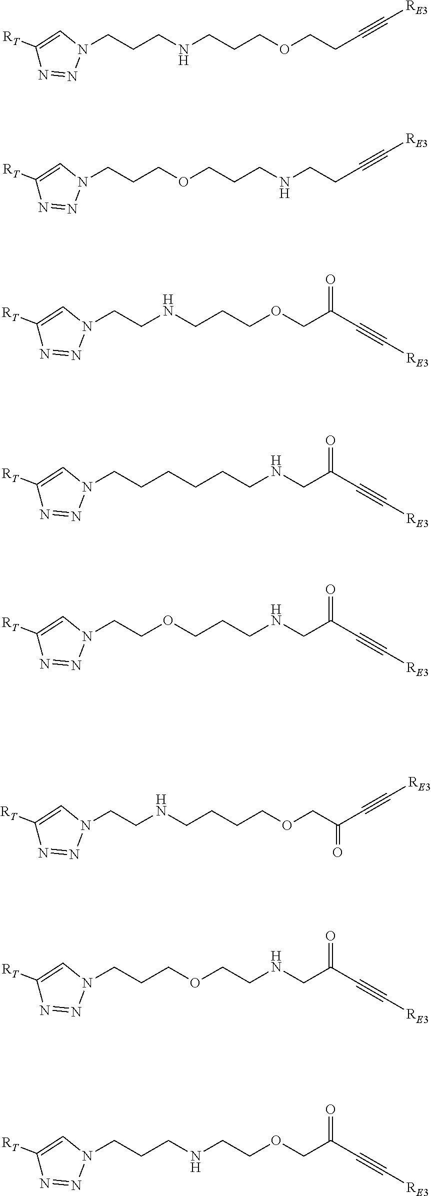

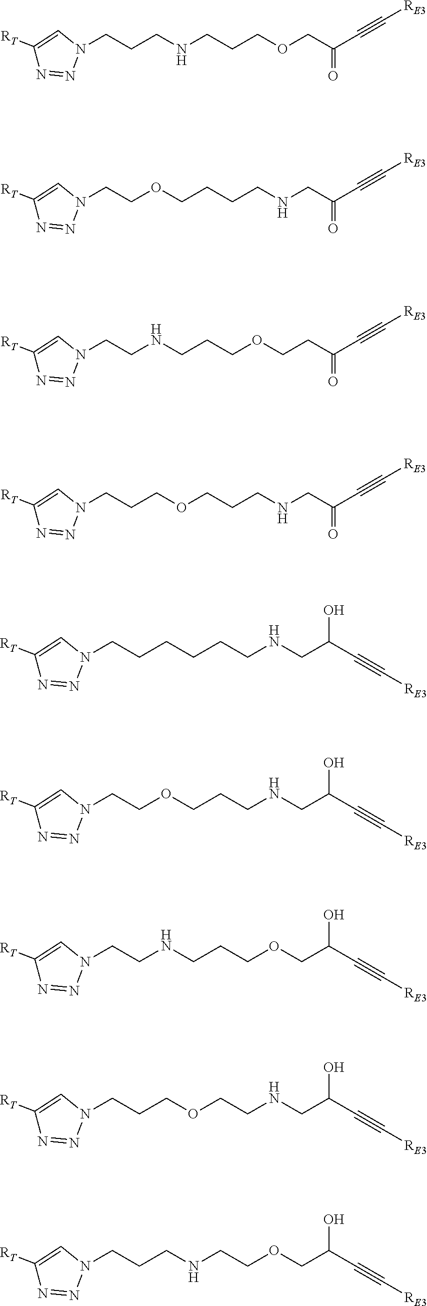

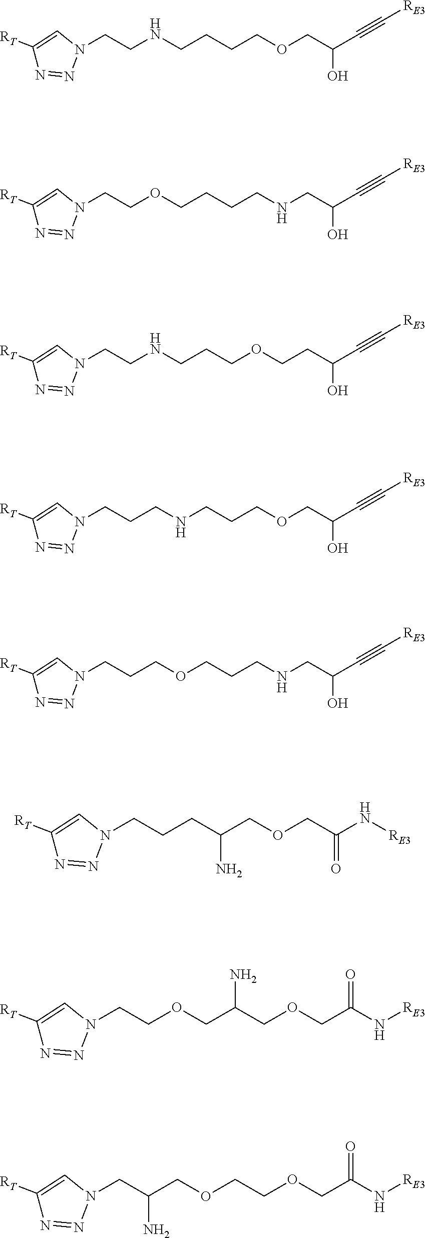

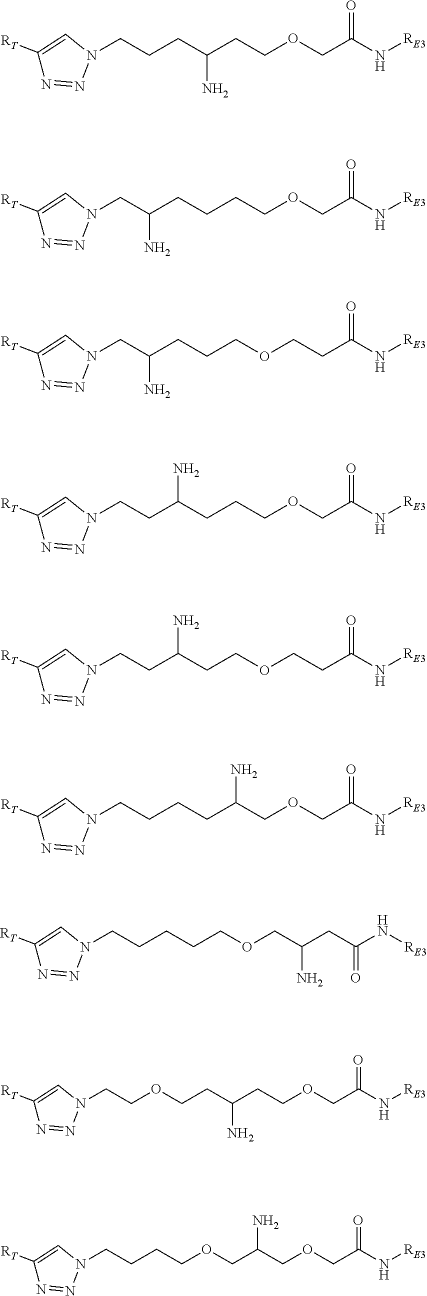

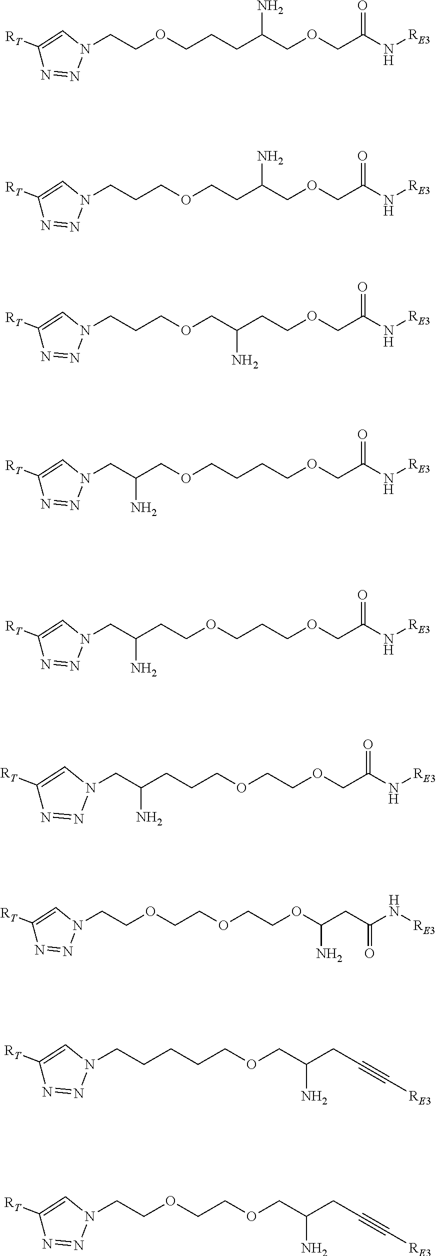

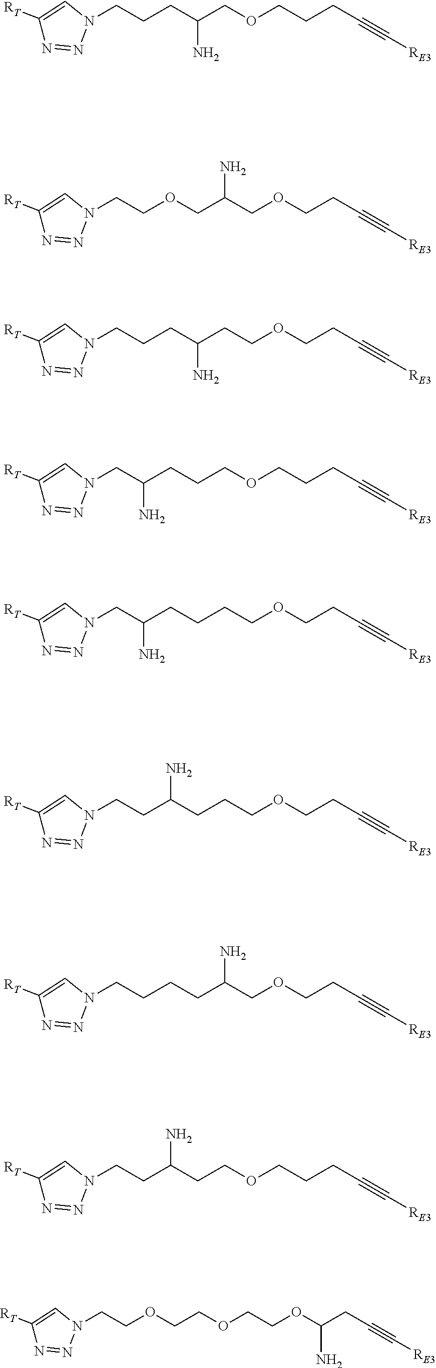

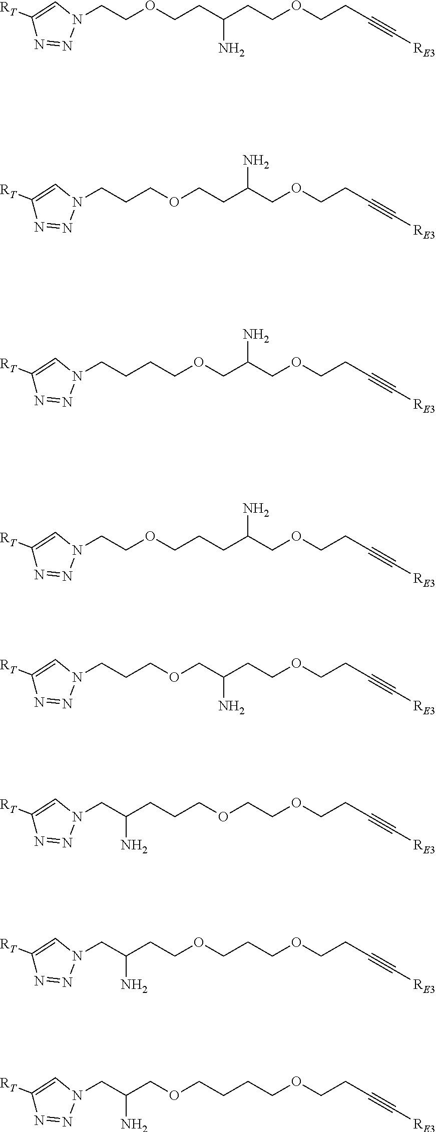

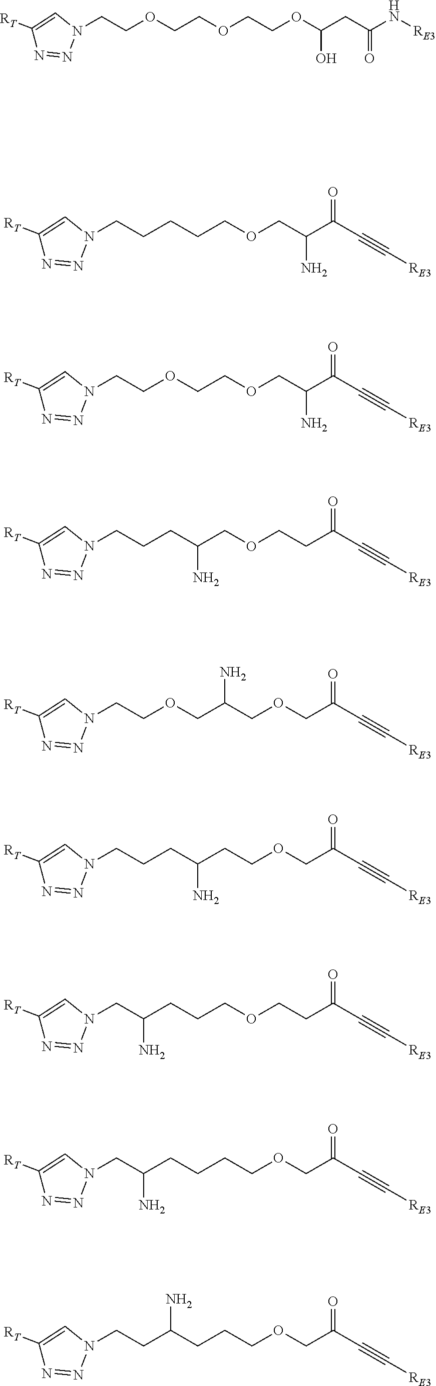

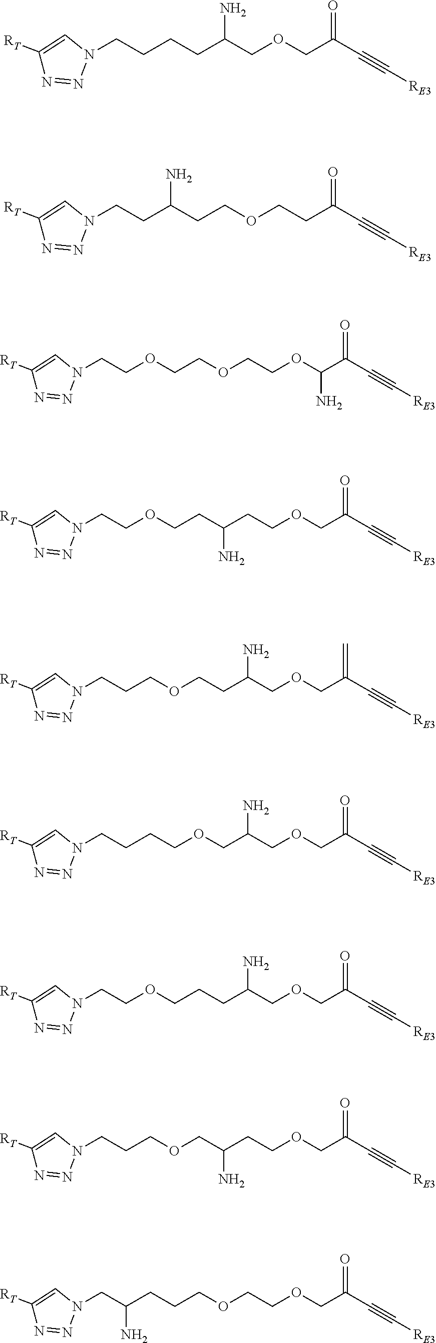

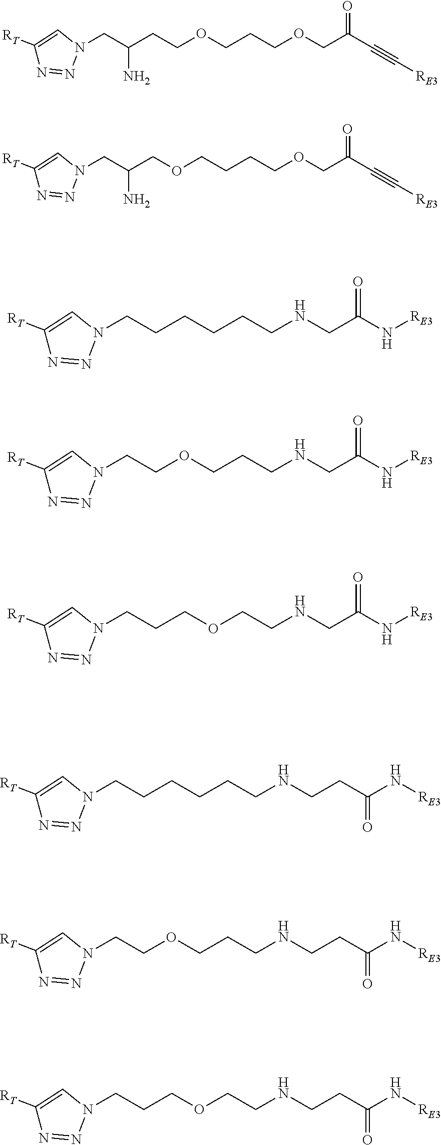

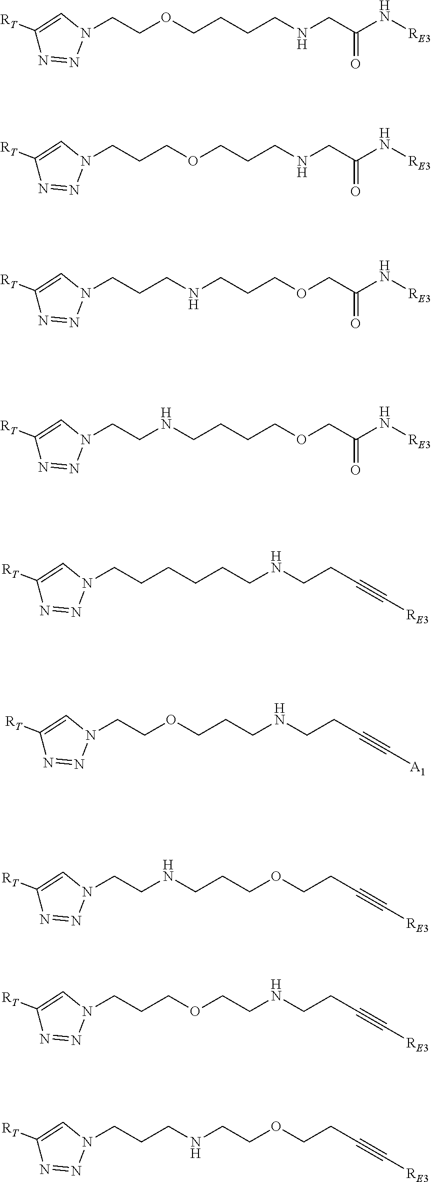

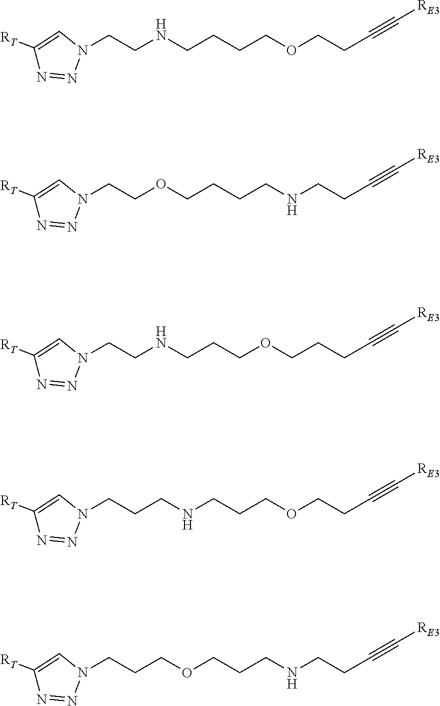

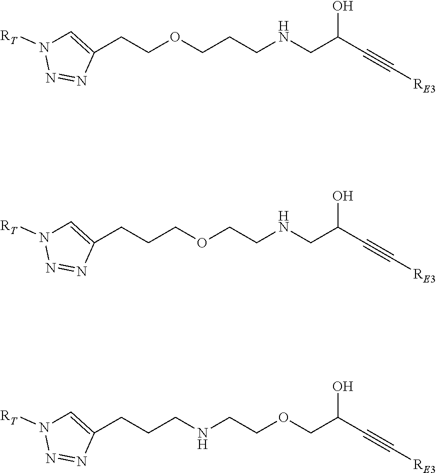

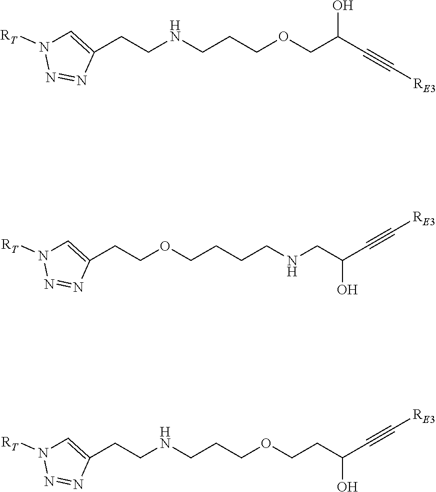

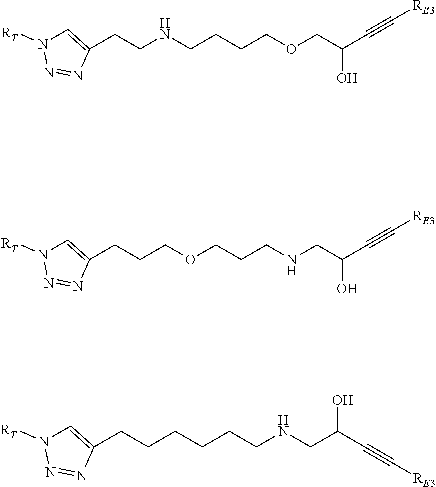

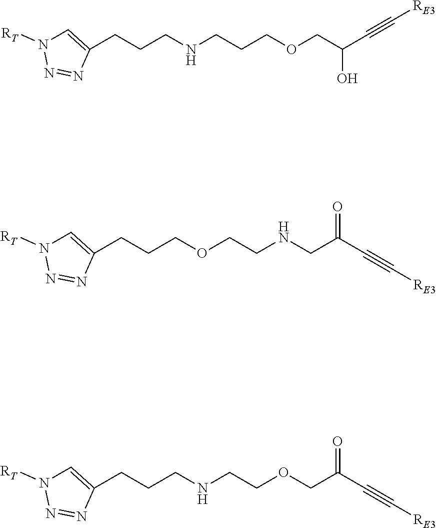

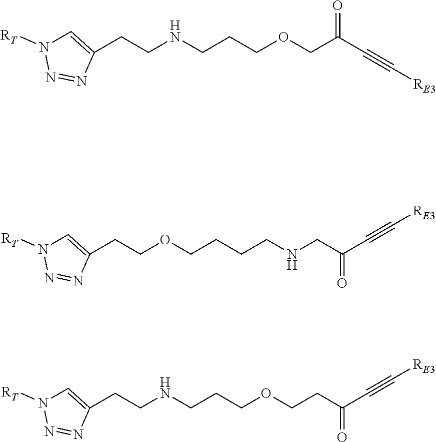

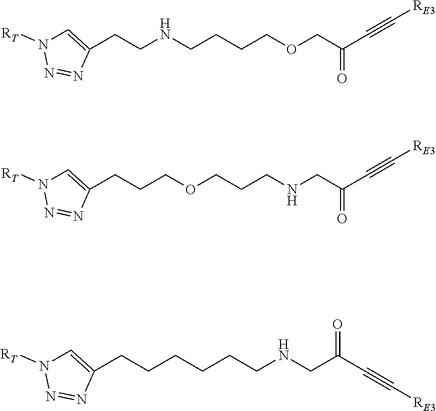

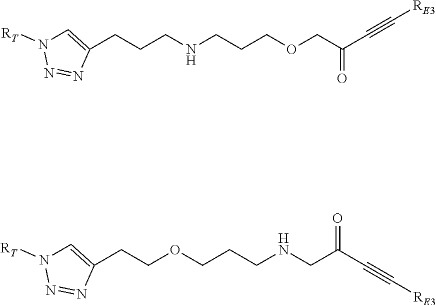

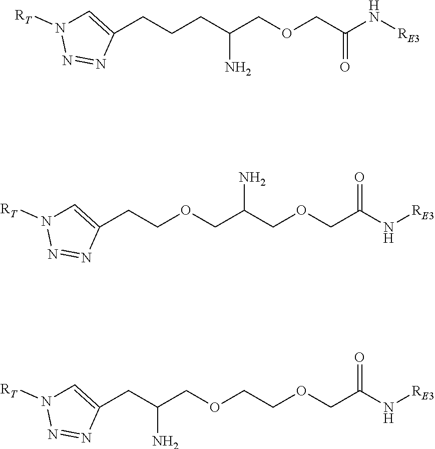

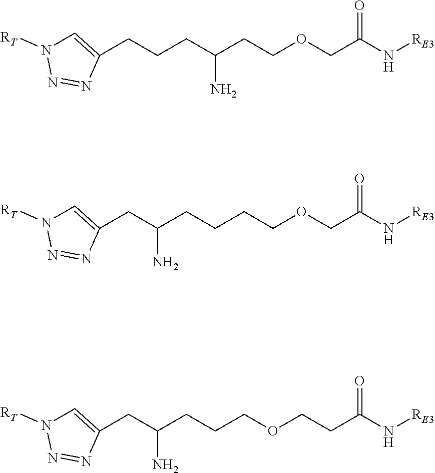

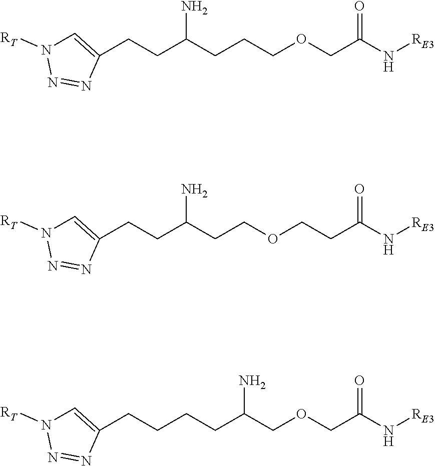

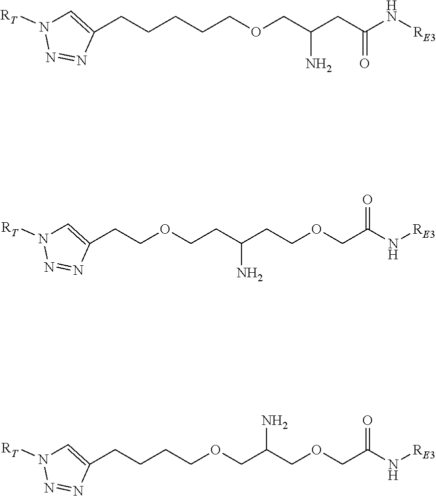

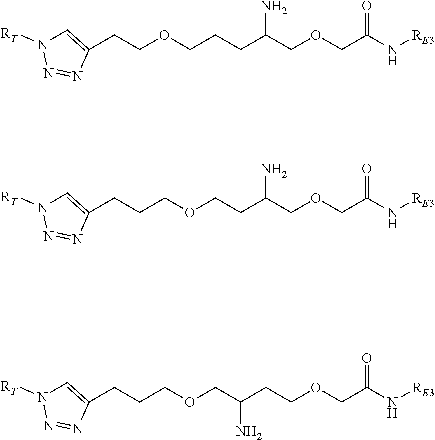

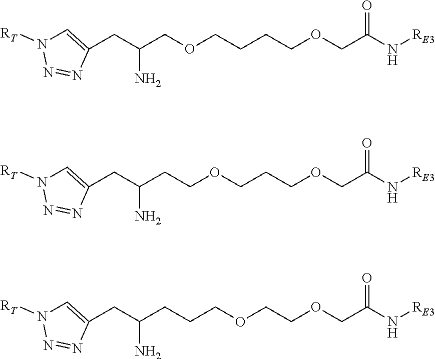

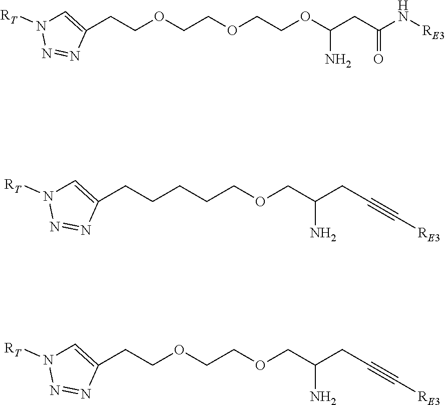

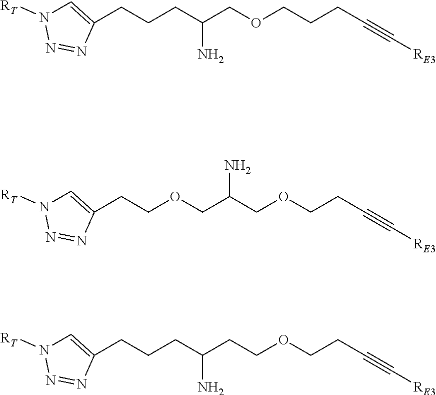

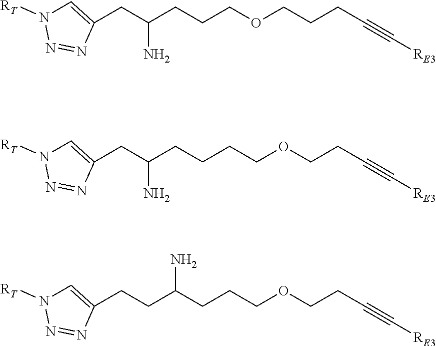

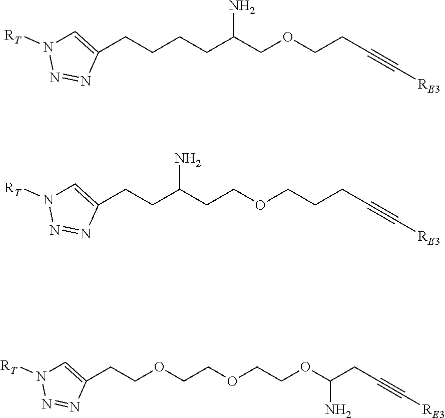

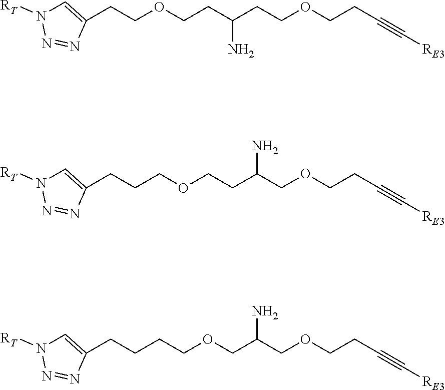

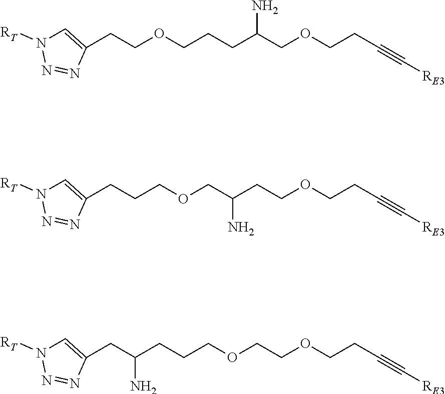

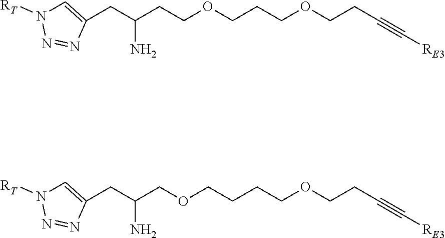

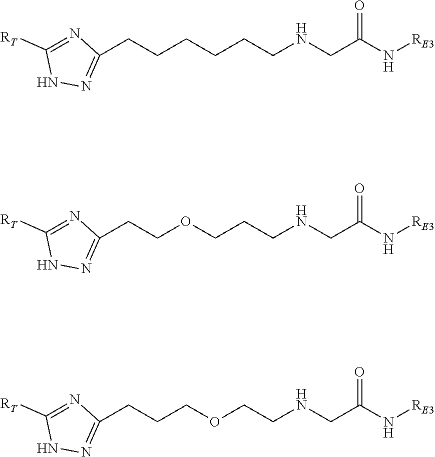

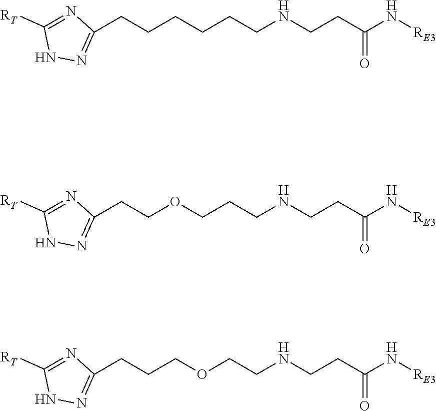

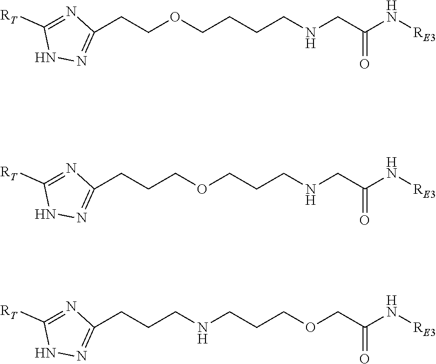

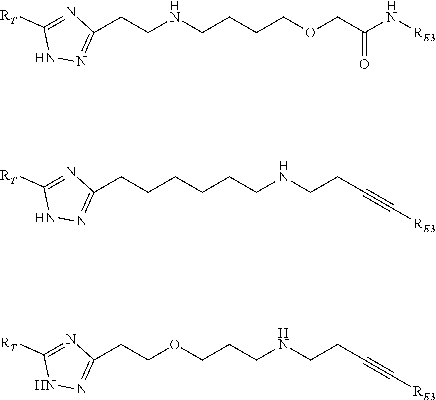

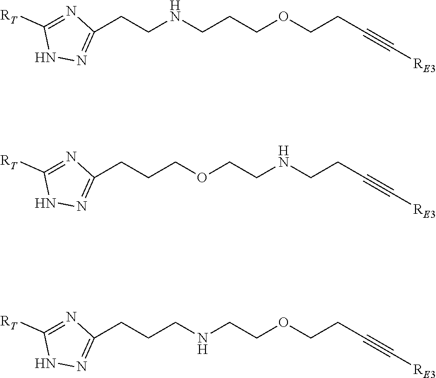

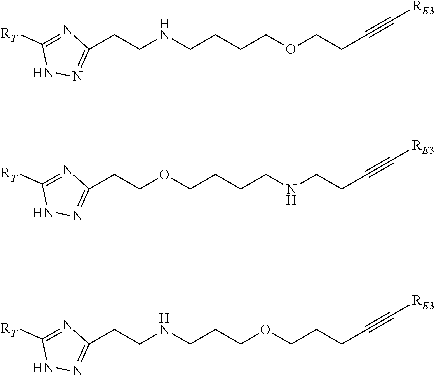

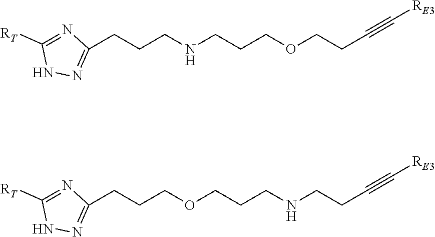

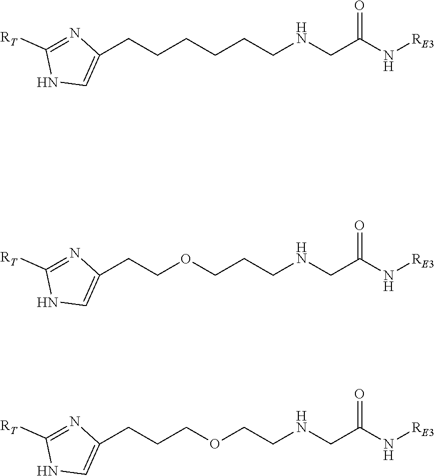

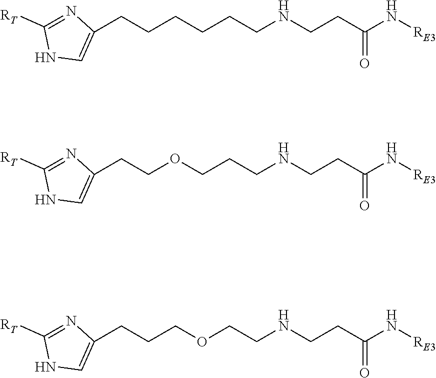

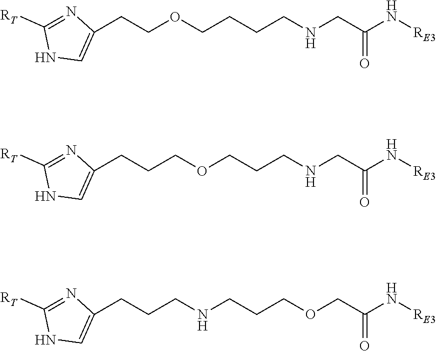

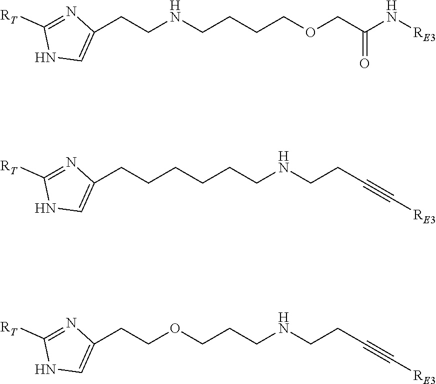

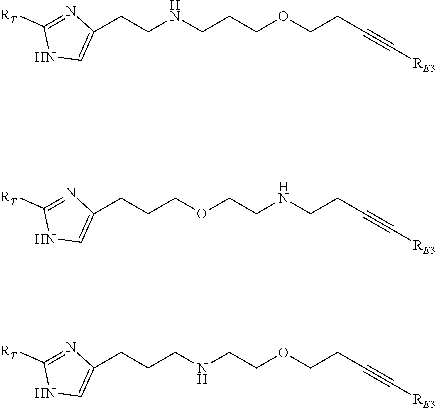

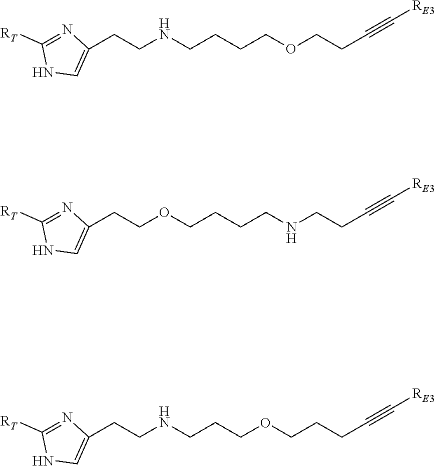

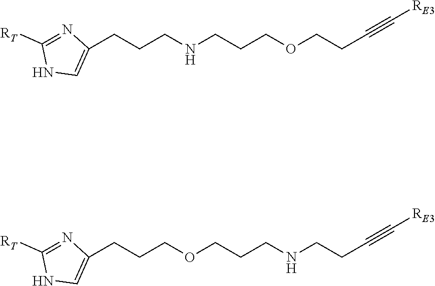

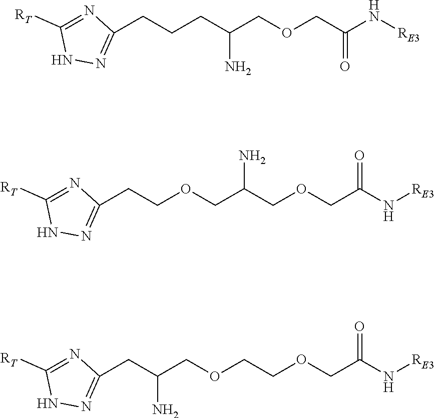

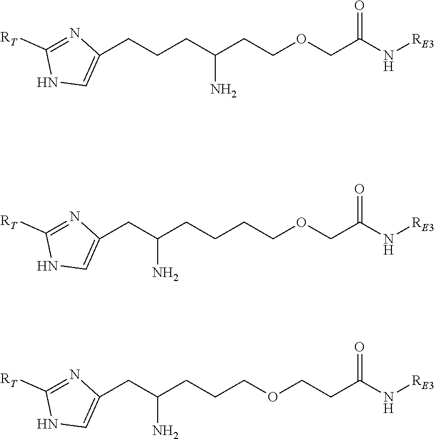

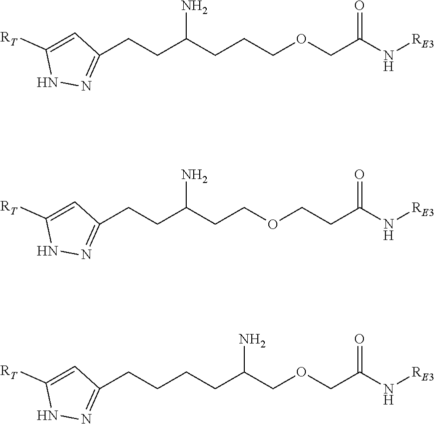

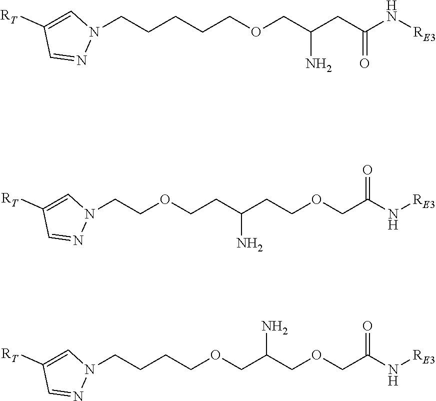

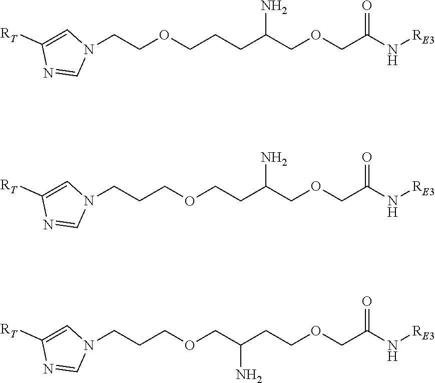

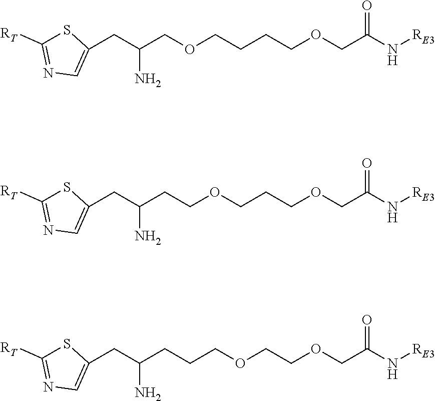

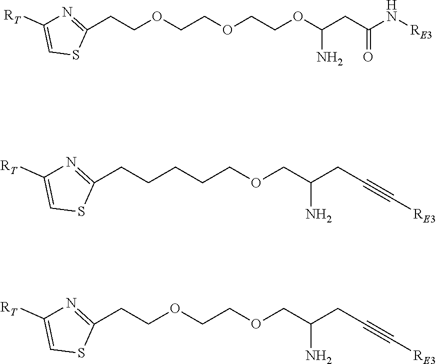

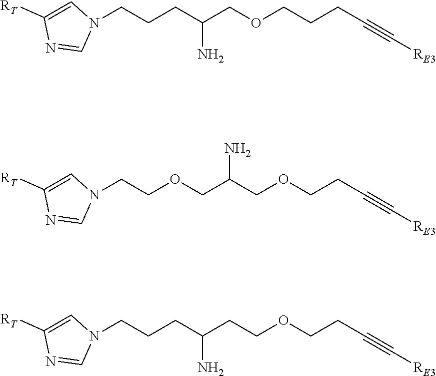

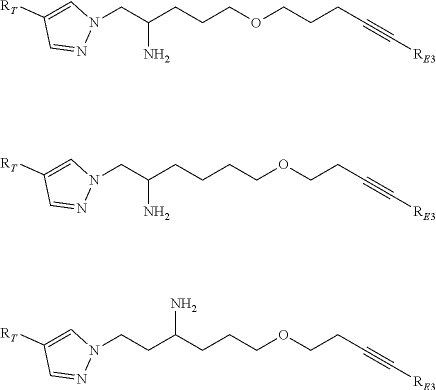

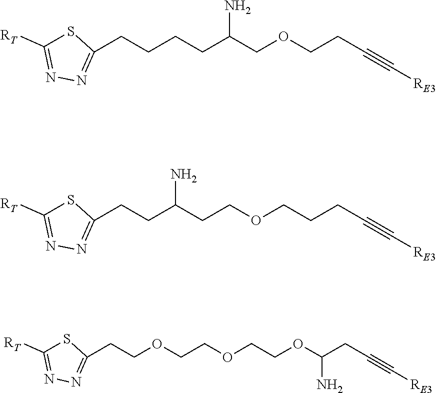

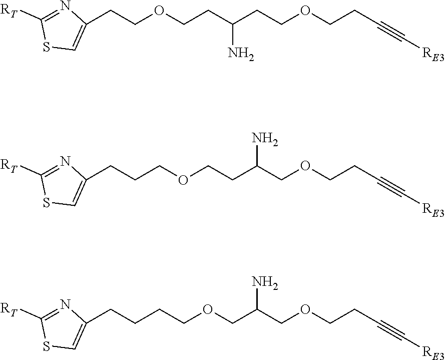

wherein R_{T} and R_{E3} are defined as above.

### ACTED

In the present invention, when the target molecule is an antibody or peptide, or cyclic peptide, or folate receptor ligand, or HSP90 ligand, or other extracellular target protein ligand, the conjugate of the present invention also can be referred to as ACTED or ACTED molecule or ACTED compound for short.

Some ACTED compounds are listed below:

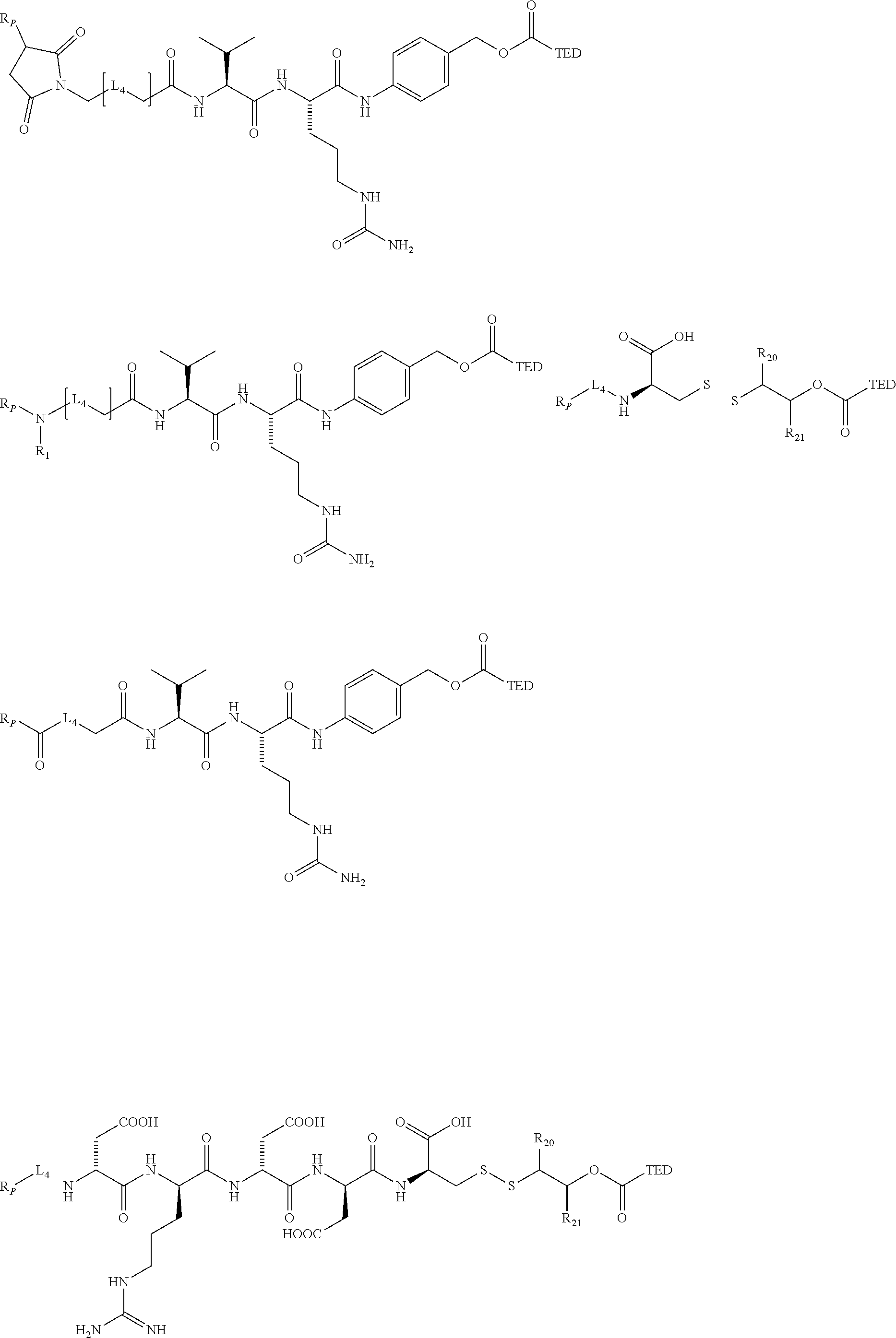

wherein TED refers to a monovalent group formed by the loss of the group on N of conjugate of formula I or TED compound of formula VI;
R_{P}, L4 are as defined as above.

In a specific embodiment, the examples of ACTED of the present invention include but are not limit to the compound or conjugate selected from the group consisting of:

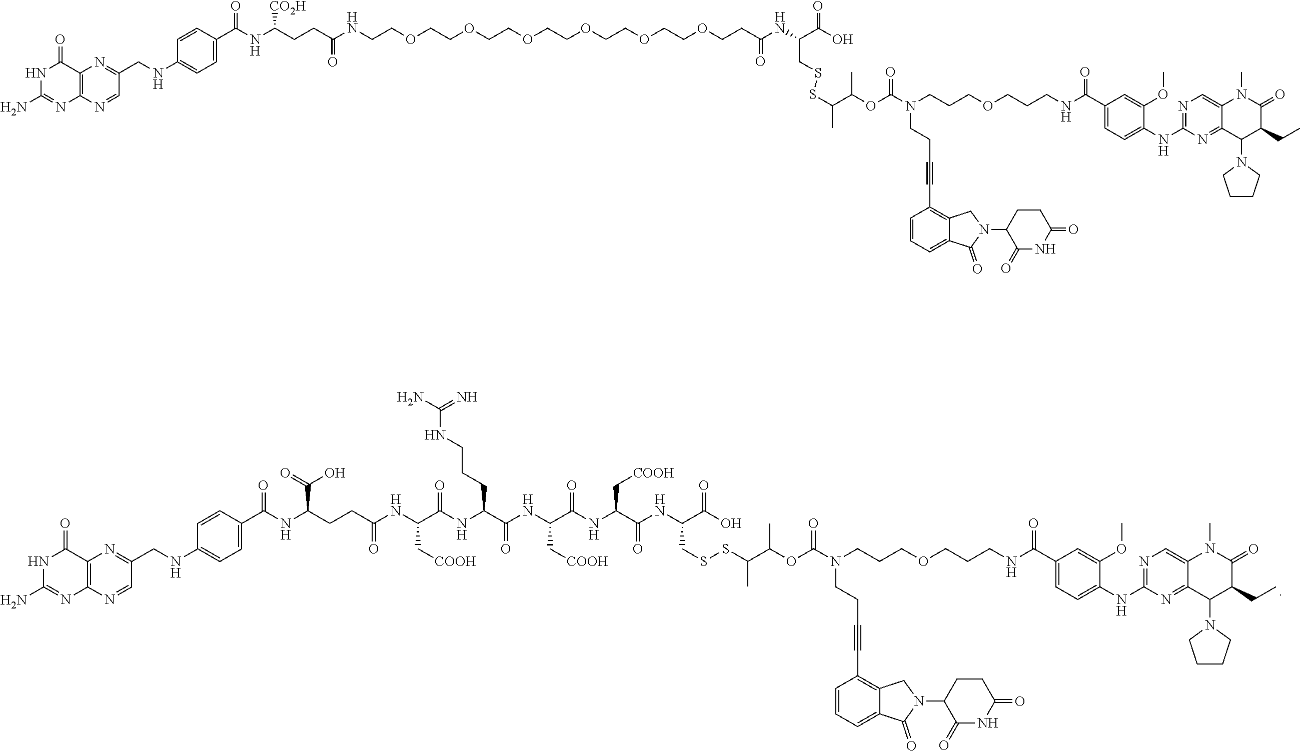

The main advantages of the present invention include:

(a) The conjugate TED of the present invention has high activity on tumor cells, has selectivity on cells and has good safety.

(b) The conjugate TED of the present invention can exert the effect of inhibiting cell proliferation in a catalytic amount. The intracellular degradation of target proteins can be circulated to reduce the dose and prolong the dosing cycle to achieve safe and effective anti-tumor effects.

(c) The conjugate TED of the present invention, the linker (L1) portion of which carries an active site that can be linked to a drug delivery vehicle (e.g., antibody, peptide, other small molecule ligands).

The present invention was further described hereafter in combination with specific examples. It should be understood that these examples are only used to illustrate and not to limit the scope of the invention. The experimental methods without specific conditions in the following examples generally follow the conventional conditions or the conditions suggested by the manufacturer. Unless otherwise stated, percentages and parts are percentages by weight and parts by weight.

Unless otherwise specified, the starting materials or compounds used in examples are commercially available or can be prepared by methods known to those skilled in the art.

### PREPARATION EXAMPLE

Unless otherwise specified, the starting materials or compounds used in examples are commercially available or can be prepared by methods known to those skilled in the art.

### General Method

### General Method 1: Synthesis of Compound P1.1-Linker-Ligand A

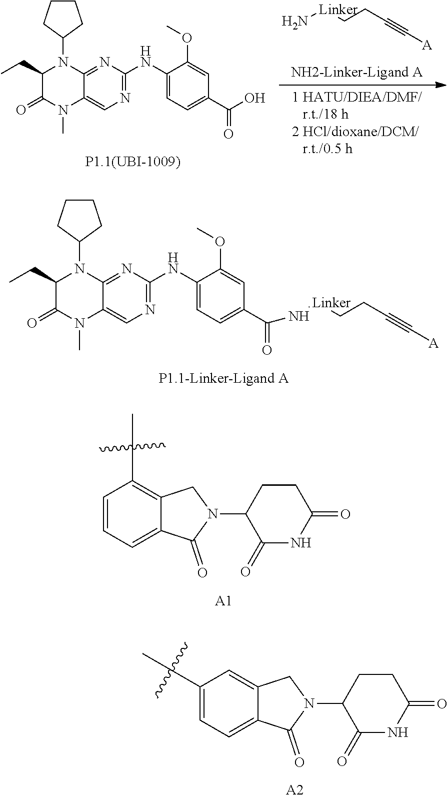

In formula, A is a structure shown in A1 or A2. Under N₂ protection, compound P1.1 (20 mg, 1 eq.), Linker-Ligand A (1 eq.), HATU (2 eq.) and DIEA (3 eq.) are dissolved in DMF (2 mL), and reacted at room temperature for 18 hours. The reaction solution is poured into 5 mL of water and extracted with ethyl acetate (5 mL*3). The organic phases are combined and then washed with saturated saline (10 mL*3), dried over anhydrous Na₂SO₄, concentrated under reduced pressure to obtain crude product, which is separated by preparative thin-layer chromatography silica gel plates (DCM/MeOH=10/1) to obtain target product.

The above target product is dissolved in DCM (3 mL), added with 0.5 mL (HCl/dioxane 4 M), and reacted at room temperature for 1 hour. The reaction solution is concentrated and then washed with ether (5 mL*3), and filtered to obtain target product P1.1-Linker-Ligand A as white solid.

### General Method 2: Synthesis of Compound P1.1-Linker g-Ligand A

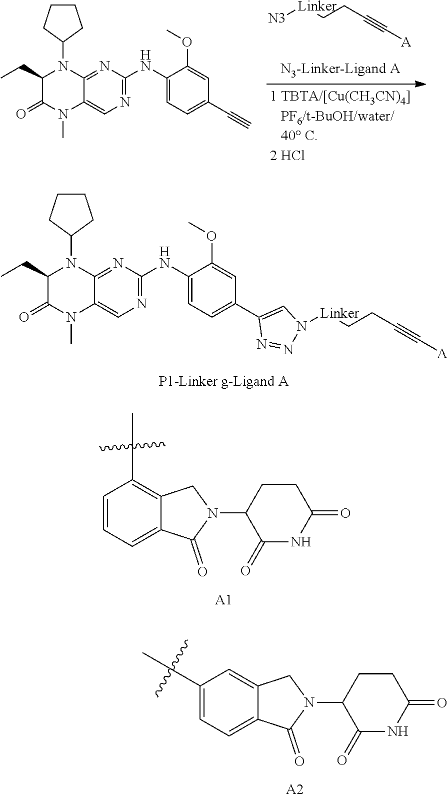

In formula, A is a structure shown in A1 or A2.

Compound (R)-8-cyclopentyl-7-ethyl-2-((4-ethynyl-2-methoxyphenyl)amino)-5-methyl-7,8-dihydropteridin-6(5H)-one (1 eq), N3-linker-Ligand A (1 eq.), TBTA (1 eq.), and [Cu(CH₃CN)₄]PF₆ (Cat.) are dissolved in tert-butanol (5 mL) and water, and the mixture is reacted at room temperature for 16 hours to 4 days. The reaction solution is concentrated under reduced pressure and then purified by silica gel column (MeOH/DCM=10%) to obtain a compound as white solid.

The above target product is dissolved in DCM (3 mL), added with 0.5 mL (HCl/dioxane 4 M), and reacted at room temperature for 1 hour. The reaction solution is concentrated and washed with ether (5 mL*3), and filtered to obtain target product P1-Linker-Ligand A as white solid.

### General Method 3: Synthesis of Compound P1-Linker-Ligand A

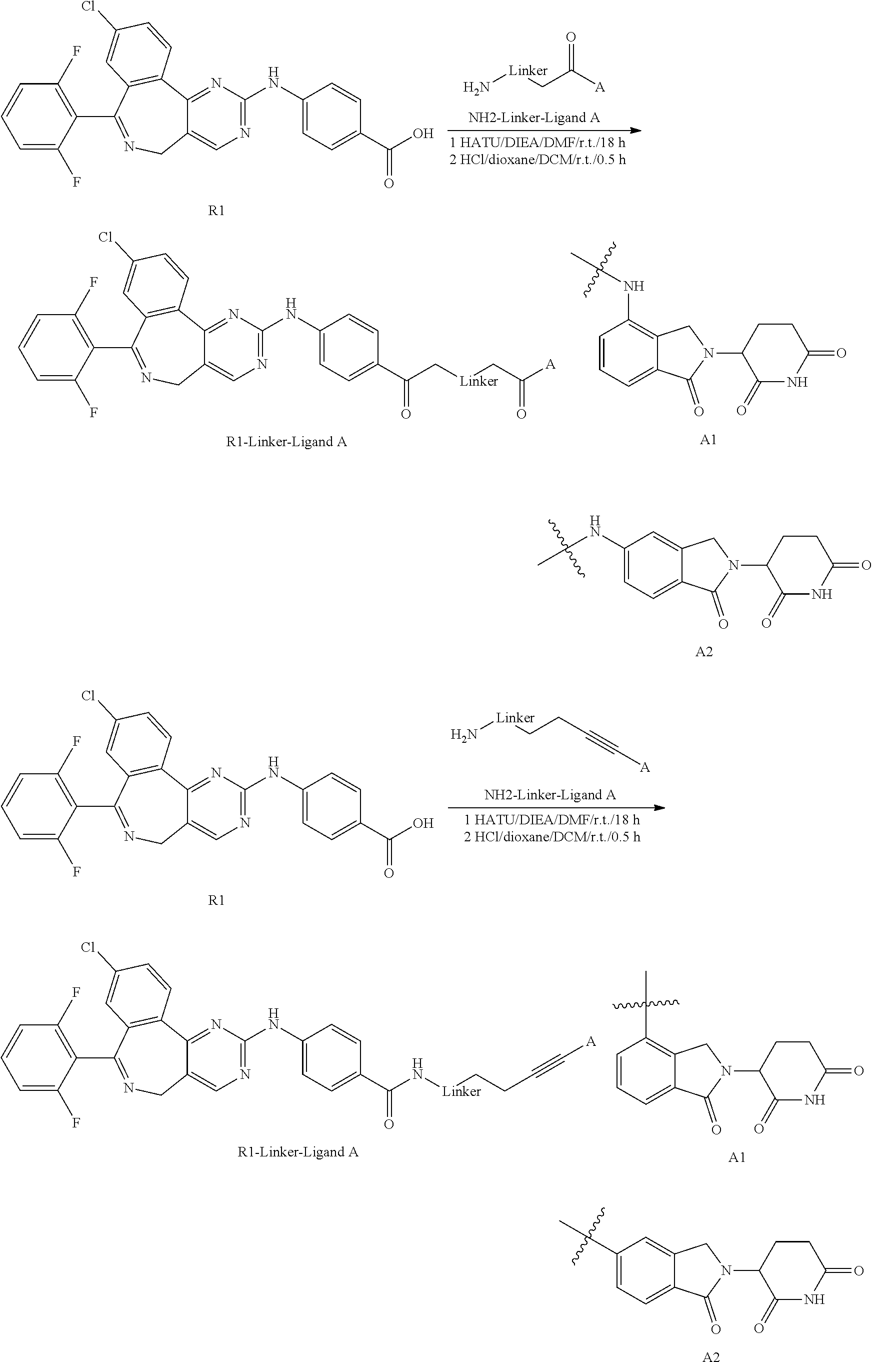

In formula, A is a structure shown in A1 or A2.

After the addition of compound R1/R2 (20 mg, 1 eq.), Linker-Ligand A (1 eq.), nitrogen protection for 18 hours. The reaction solution is poured into 5 mL of water and extracted with ethyl acetate (5 mL*3). The organic phases are combined and then washed with saturated saline (10 mL*3), dried over anhydrous Na₂SO₄, concentrated by rotary evaporation under reduced pressure to obtain crude product, which is separated by preparative thin-layer chromatography silica gel plates (DCM/MeOH=10/1) to obtain target product.

The above target product is dissolved in DCM (3 mL), added with 0.5 mL (HCl/dioxane 4 M), and reacted at room temperature for 1 hour. The reaction solution is concentrated and then washed with ether (5 mL*3), and filtered to obtain target product R1-Linker-Ligand A as white solid.

### General Method 4: Synthesis of Compound R2-Linker-Ligand A

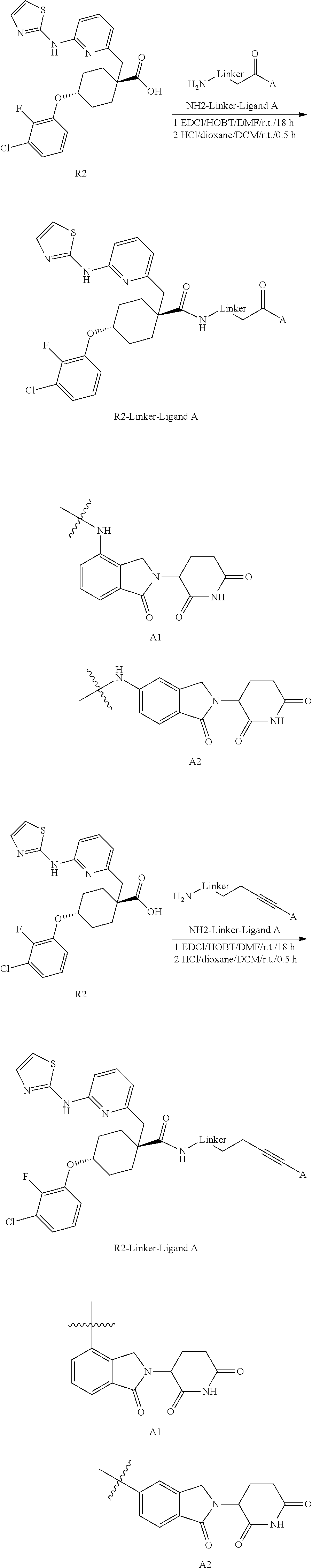

In formula, A is a structure shown in A1 or A2.

Compound R1/R2 (20 mg, 1 eq.), Linker-Ligand A (1 eq.), EDCI (2 eq.), and HOBT (2 eq.) are dissolved in DIEA (3 eq.) in DMF (2 mL), after addition, the reaction is carried out at room temperature under nitrogen protection for 18 hours. The reaction solution is poured into 5 mL of water and extracted with ethyl acetate (5 mL*3). The organic phases are combined and wawashed with saturated saline (10 mL*3), dried over anhydrous Na₂SO₄, concentrated by rotary evaporation under reduced pressure to obtain crude product, which is separated by preparative thin-layer chromatography silica gel plates (DCM/MeOH=10/1) to obtain target product.

The above target product is dissolved in DCM (3 mL), added with 0.5 mL (HCl/dioxane 4 M), and reacted at room temperature for 1 hour. The reaction solution is concentrated and washed with ether (5 mL*3), and filtered to obtain target product R2-Linker-Ligand A as white solid.

### General Method 5: Synthesis of Compound R1/R2-Linker-Ligand A

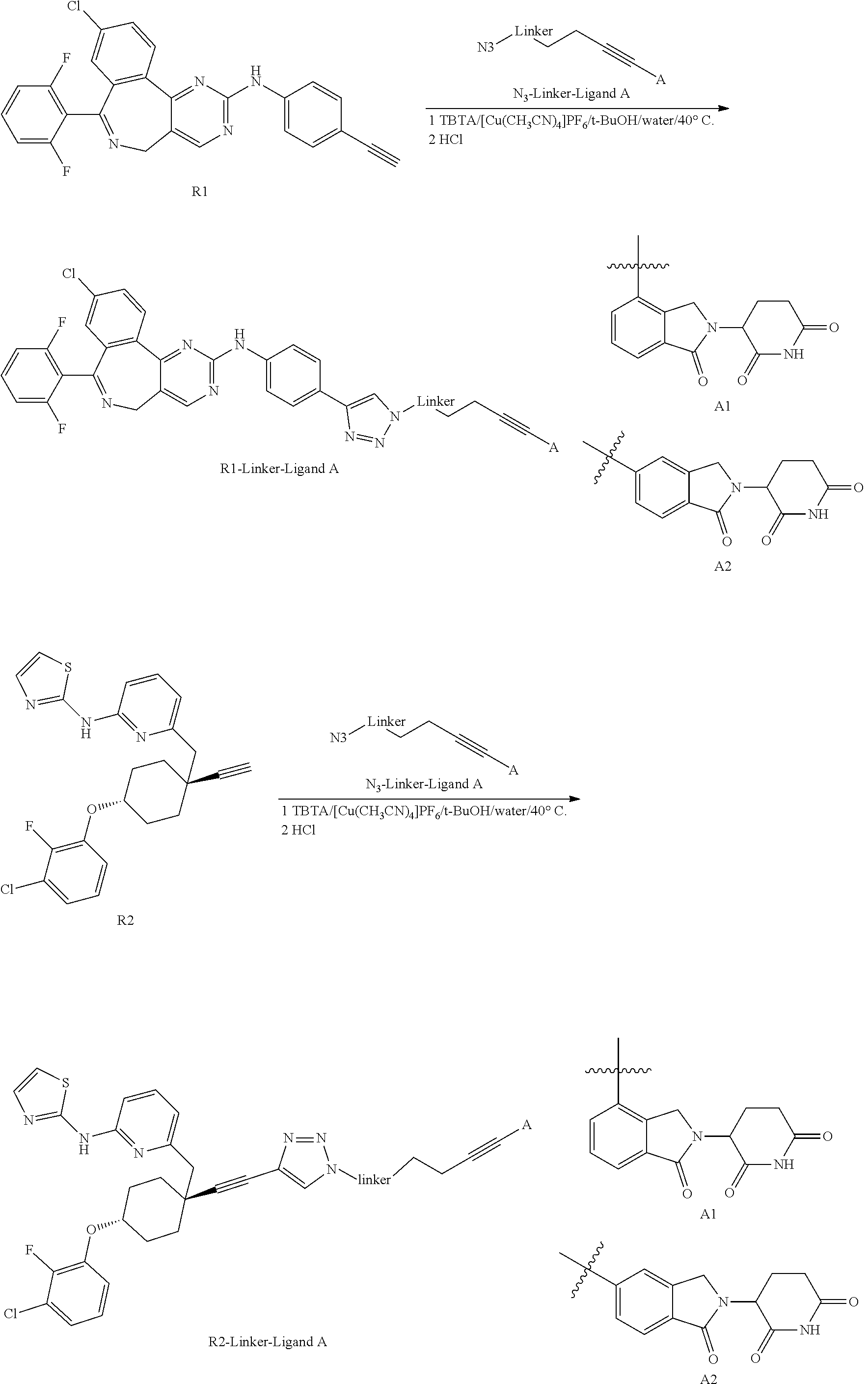

In formula, A is a structure shown in A1 or A2.

Compound R1/R2 (1 eq.), N3-Linker-Ligand A (1 eq.), TBTA (1 eq.), and [Cu(CH₃CN)₄]PF₆(Cat.) are dissolved in t-BuOH (5 mL) and water (1 mL), and reacted at room temperature for 16 hours to 4 days. After the completion of reaction, the reaction is concentrated to obtain crude product, which is purified by silica gel column to obtain white solid.

The above target product is dissolved in DCM (3 mL), added with 0.5 mL (HCl/dioxane 4 M), and reacted at room temperature for 1 hour. The reaction solution is concentrated and then washed with ether (5 mL*3), and filtered to obtain target product R1/R2-Linker-Ligand A as white solid.

### General Method 6: Synthesis of Compound M-Linker-Ligand A

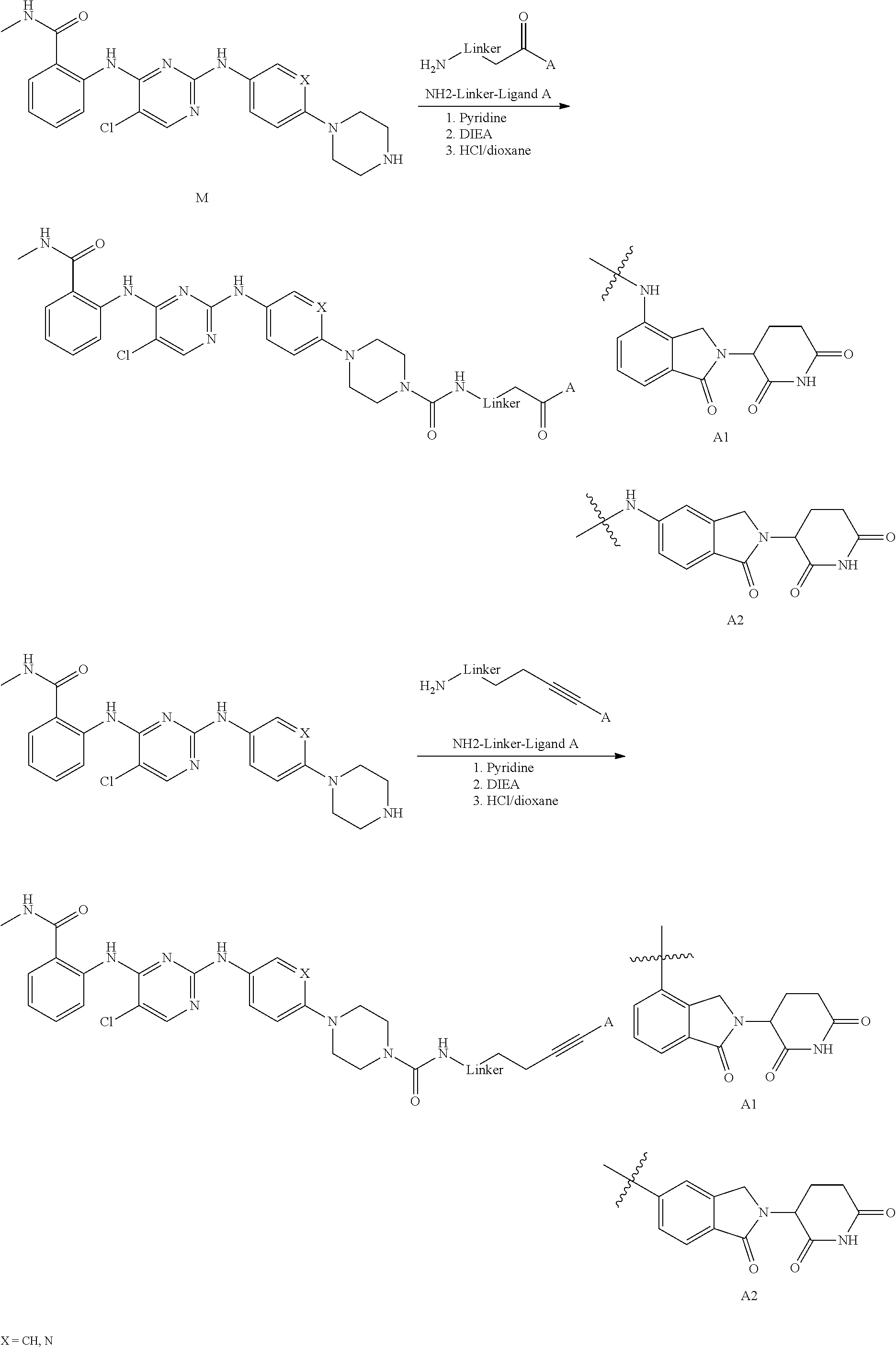

In formula, A is a structure shown in A1 or A2.

Compound NH2-Linker-Ligand A (1 eq.) is dissolved in pyridine, then added with bis(p-nitrophenyl)carbonate (1 eq), and reacted at room temperature for 2 hours. A yellow reaction solution is then obtained by adding M (1 eq) and DIPEA, followed by reacting at room temperature for 1 hour. The reaction solution is concentrated and then purified by silica gel column to obtain white solid.

The above target product is dissolved in DCM (3 mL), added with 0.5 mL (HCl/dioxane 4 M), and reacted at room temperature for 1 hour. The reaction solution is concentrated and then washed with ether (5 mL*3), and filtered to obtain target product M-Linker-Ligand A as white solid.

### General Method 7: Synthesis of Compound R3-Linker-Ligand E

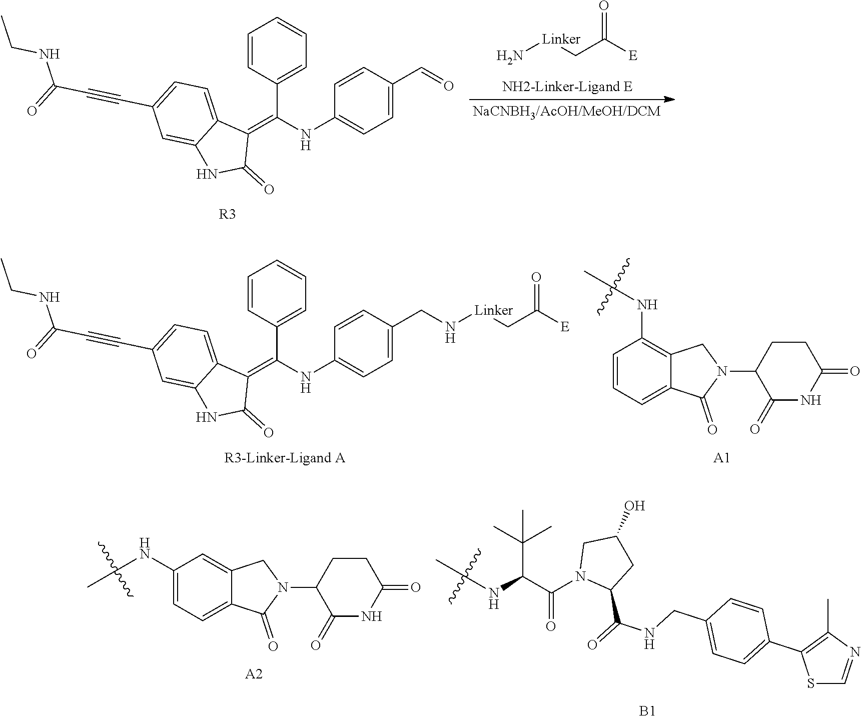

In formula, E is a structure of A1, A2 or B1.

Compound R3 (20 mg, 1 eq.), Linker-Ligand E (2 eq.) and a catalytic amount of AcOH (1 drop) are dissolved in methanol/dichloromethane=1/10 (10 mL) and reacted at room temperature for 18 hours. Then NaCNBH₃ (3 eq.) is added and the reaction continues at room temperature for 3 hours. The reaction solution is concentrated and then washed once with water (5 mL), extracted twice with ethyl acetate (10 mL), and the organic phase is concentrated to obtain the target product R3-Linker-Ligand E.

### PREPARATION EXAMPLE

### Synthesis of Compound UB-180937

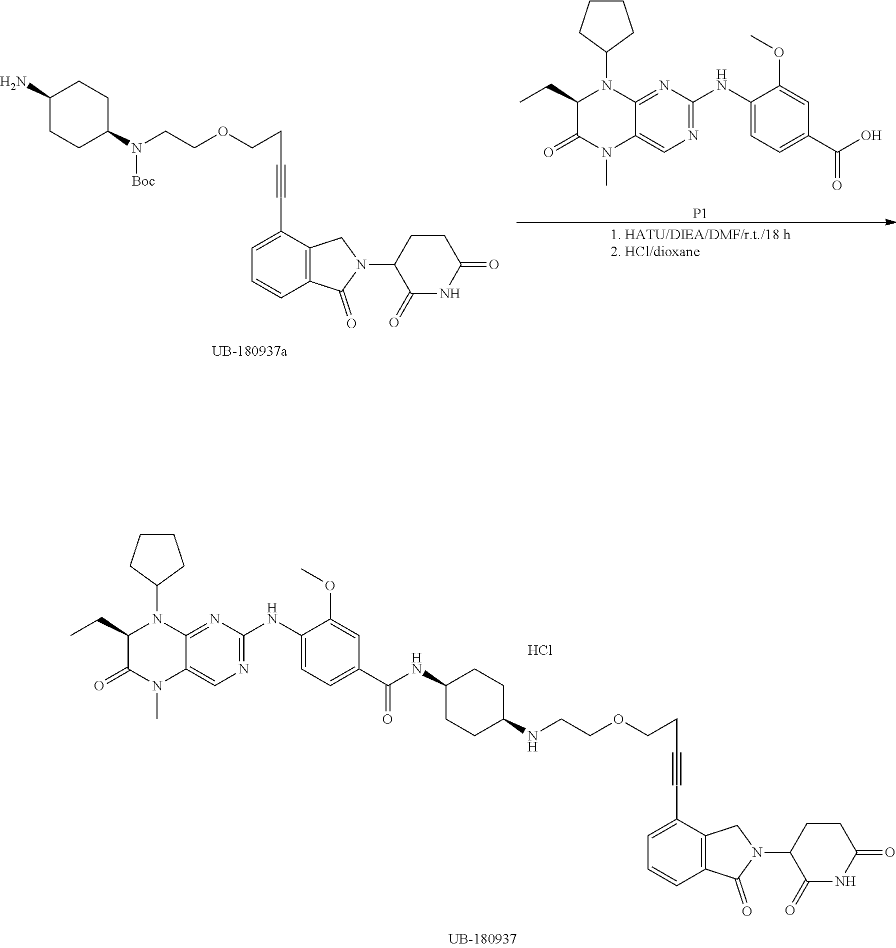 [SMILES: CC(C)(C)OC(=O)N(CCOCCC#Cc1cccc2c1CN(C1CCC(=O)NC1=O)C2=O)[C@H]1CC[C@@H](N)CC1 ; CC[C@@H]1C(=O)N(C)c2cnc(Nc3ccc(C(=O)O)cc3OC)nc2N1C1CCCC1 ; "P1" ; CC[C@@H]1C(=O)N(C)c2cnc(Nc3ccc(C(=O)N[C@H]4CC[C@@H](NCCOCCC#Cc5cccc6c5CN(C5CCC(=O)NC5=O)C6=O)CC4)cc3OC)nc2N1C1CCCC1 ; Cl]

Synthesised using a method similar to General Method 1. ¹H NMR (400 MHz, DMSO-d₆) δ 11.01 (s, 1H), 9.70 (s, 1H), 8.98 (s, 2H), 7.95 (d, J=5.4 Hz, 1H), 7.87-7.80 (m, 2H), 7.73 (dd, J=7.6, 1.1 Hz, 1H), 7.68-7.58 (m, 3H), 7.53 (t, J=7.6 Hz, 1H), 5.16 (dd, J=13.3, 5.1 Hz, 1H), 4.51-4.45 (m, 2H), 4.33 (s, 1H), 4.17 (d, J=8.8 Hz, 1H), 3.94 (s, 4H), 3.81 (t, J=5.3 Hz, 2H), 3.70 (t, J=6.7 Hz, 2H), 3.18 (d, J=26.3 Hz, 6H), 2.99-2.88 (m, 1H), 2.80 (t, J=6.7 Hz, 2H), 2.59 (d, J=17.8 Hz, 1H), 2.46 (dd, J=13.1, 4.2 Hz, 1H), 2.09-1.73 (m, 13H), 1.63-1.39 (m, 6H), 0.76 (t, J=7.4 Hz, 3H). LCMS [M/2+1]⁺=431.1

### Synthesis of Compound UB-180934

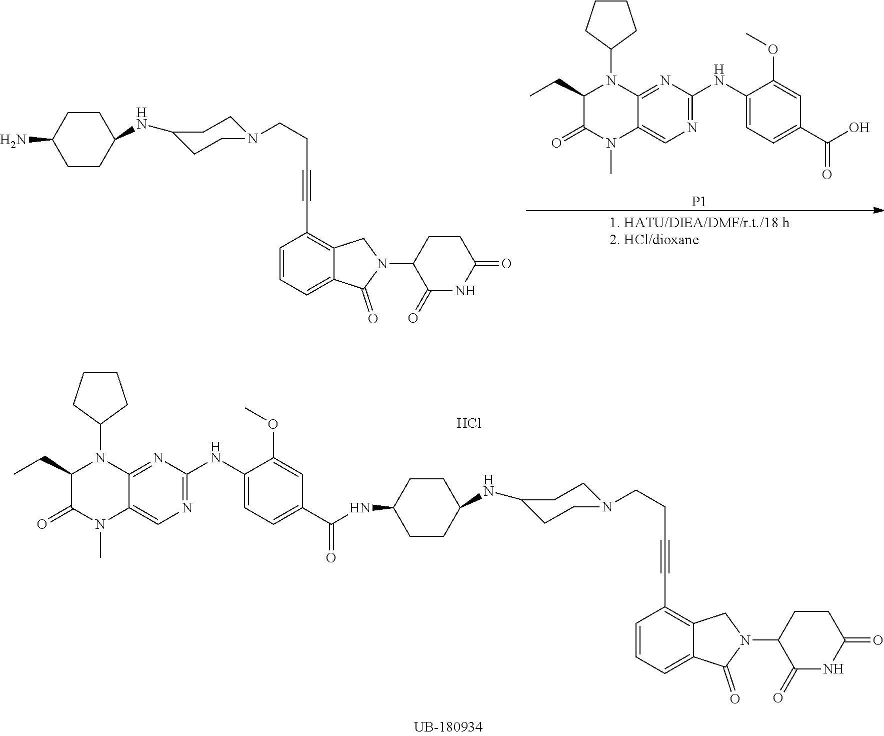 [SMILES: N[C@H]1CC[C@@H](NC2CCN(CCC#Cc3cccc4c3CN(C3CCC(=O)NC3=O)C4=O)CC2)CC1 ; CC[C@@H]1C(=O)N(C)c2cnc(Nc3ccc(C(=O)O)cc3OC)nc2N1C1CCCC1 ; "P1" ; CC[C@@H]1C(=O)N(C)c2cnc(Nc3ccc(C(=O)N[C@H]4CC[C@@H](NC5CCN(CCC#Cc6cccc7c6CN(C6CCC(=O)NC6=O)C7=O)CC5)CC4)cc3OC)nc2N1C1CCCC1 ; Cl]

Synthesised using a method similar to General Method 1. LCMS [M+1]⁺=899.7

¹H NMR (400 MHz,) δ 11.02 (s, 1H), 8.41 (d, J=8.5 Hz, 1H), 7.84 (d, J=12.8 Hz, 2H), 7.69 (t, J=14.8 Hz, 2H), 7.57-7.51 (m, 2H), 7.51-7.44 (m, 2H), 5.17 (dd, J=13.2, 5.2 Hz, 1H), 4.46 (d, J=17.7 Hz, 1H), 4.39-4.28 (m, 2H), 4.25 (dd, J=7.7, 3.6 Hz, 1H), 3.95 (d, J=7.9 Hz, 4H), 3.25 (s, 3H), 2.96 (d, J=18.8 Hz, 2H), 2.72-2.63 (m, 2H), 2.59 (s, 1H), 2.43 (s, 1H), 2.06 (d, J=18.5 Hz, 7H), 1.94-1.71 (m, 11H), 1.71-1.50 (m, 9H), 0.77 (t, J=7.4 Hz, 3H).

### Synthesis of Compound UB-180961

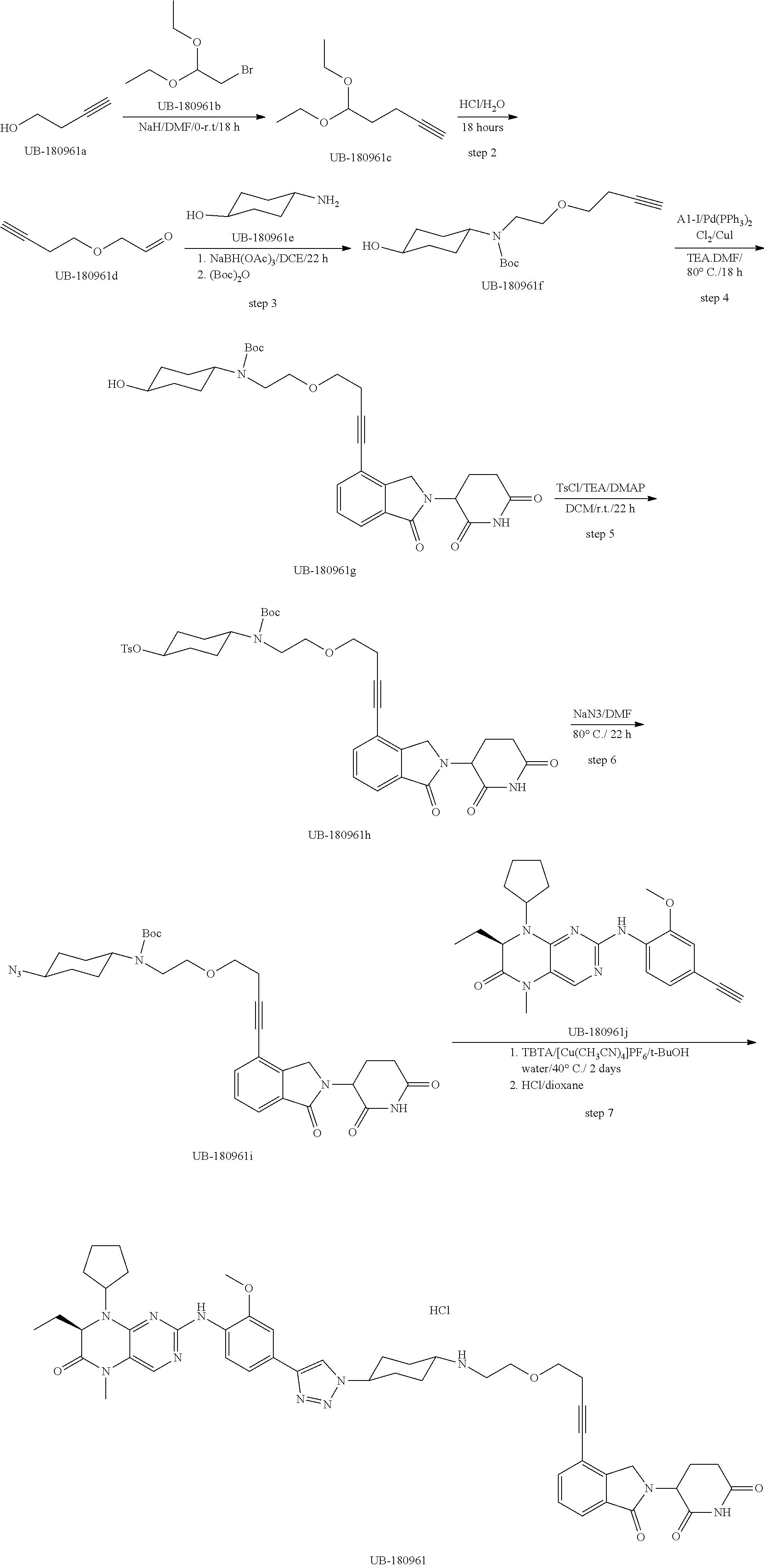

### Step 1: UB-180961c

Compound UB-180961a (20 g, 71.1 mmol) was dissolved in dry DMF (80 mL) and cooled to 0° C., then NaH (16.8 g, 107 mmol) was added to the reaction solution. After half an hour, UB-180961b (21.1 g, 107 mmol) was dissolved in dry DMF (20 mL) and added to the reaction solution, and the mixture was reacted at room temperature overnight. The reaction solution was added with ice water (100 mL) and extracted three times with EtOAc (60 mL); the organic phases were combined and then separated by column chromatography (PE/EtOAc=0-100%) to afford the target product UB-180961c (25 g, 47% yield) as colourless oil.

¹H NMR (400 MHz, CDCl₃) δ 4.63 (t, J=5.2 Hz, 1H), 3.72-3.52 (m, 8H), 2.47 (td, J=7.0, 2.6 Hz, 2H), 1.98 (t, J=2.7 Hz, 1H), 1.28-1.21 (m, 6H).

### Step 2: UB-180961d

Compound UB-180961c (15 g, 285.4 mmol) was dissolved in water (40 mL), then added with concentrated HCl (10 mL) and reacted at room temperature overnight. The reaction solution was extracted with DCM (50 mL) 3 times and the organic phase was dried over anhydrous sodium sulfate and concentrated to give the target product UB-180961d (7.6 g) as colourless oil. The crude product was directly used in the next reaction.

¹H NMR (400 MHz, CDCl₃) δ 9.74 (d, J=0.8 Hz, 1H), 4.15 (d, J=0.8 Hz, 2H), 3.72-3.67 (m, 2H), 2.54 (t, J=2.7 Hz, 2H), 2.02 (t, J=3.4 Hz, 1H).

### Step 3: UB-180961f

Compounds UB-180961d (7.8 g, 68 mmol) and UB-180961e (7.6 g, 68 mmol) were dissolved in DCE (100 mL) and then reacted at room temperature for 1 h. The reaction was further added with NaBH(OAc)3 (29.6 g, 136 mmol), and continued to react overnight at room temperature. The reaction solution was added with TEA (5 mL, sat) and Boc₂O (6 g, 23.8 mmol) and then reacted for 18 h at room temperature. The reaction solution was extracted twice with EtOAc (15 mL), the organic phase was dried over anhydrous Na₂SO₄ and then separated by column chromatography (PE/EtOAc=0-100%) to afford the target product UB-180961f (4.6 g, 57% yield) as yellow oil.

¹H NMR (400 MHz, CDCl₃) δ 4.14 (ddd, J=28.9, 14.7, 7.4 Hz, 5H), 2.67 (t, J=11.7 Hz, 2H), 1.82 (dd, J=14.1, 7.0 Hz, 2H), 1.70 (d, J=12.3 Hz, 2H), 1.59 (d, J=18.5 Hz, 4H), 1.46 (s, 9H), 1.19 (dd, J=12.2, 4.0 Hz, 2H).

### Step 4: UB-180961g

Compound UB-180961f (4.6 g, 15 mmol) and A1-I (3.7 g, 10 mmol), Pd(PPh₃)₂Cl₂ (701 mg, 1 mmol), CuI (190 mg, 1 mmol), and TEA (4.2 ml, 30 mmol) were dissolved in dry DMF (120 mL) and reacted at 80° C. overnight. The reaction solution was extracted twice with EtOAc (15 mL), the organic phase was dried over anhydrous Na₂SO₄ and then separated by column chromatography (PE/EtOAc=0-100%) to afford the target product UB-180961g (3.6 g, 57% yield) as yellow solid. ¹H NMR (400 MHz, DMSO) δ 11.00 (s, 1H), 7.72 (d, J=6.9 Hz, 1H), 7.63 (d, J=7.2 Hz, 1H), 7.53 (t, J=7.6 Hz, 1H), 5.15 (dd, J=13.3, 5.1 Hz, 1H), 4.46 (dd, J=18.0, 11.1 Hz, 2H), 4.30 (d, J=17.7 Hz, 1H), 3.62 (t, J=6.6 Hz, 2H), 3.47 (t, J=6.5 Hz, 2H), 3.30-3.15 (m, 3H), 2.98-2.87 (m, 1H), 2.71 (t, J=6.7 Hz, 2H), 2.59 (d, J=18.0 Hz, 1H), 2.44 (dd, J=13.1, 4.4 Hz, 1H), 2.07-1.98 (m, 1H), 1.79-1.77 (d, J=11.1 Hz, 2H), 1.53 (s, 4H), 1.37 (s, 9H), 1.21-1.08 (m, 2H).

### Step 5: UB-180961h

Compounds UB-180961g (3.6 g, 33.8 mmol), TEA (10.3 g, 10.2 mmol), and DMAP (12.4 g, 10.2 mmol) were dissolved in dry DMF (140 mL), then added with TsCl (14.6 g, 7.7 mmol) at 0° C. The reaction solution was warmed up to 30° C., the reacted for 15 hours. The reaction solution was extracted twice with DCM (50 mL) and the organic phase was concentrated and separated by column chromatography (PE/EtOAc=0-100%) to afford the target product UB-180961h (3.6 g, 86% yield) as white solid.

¹H NMR (400 MHz, DMSO) δ 11.01 (s, 1H), 7.78 (d, J=8.3 Hz, 2H), 7.73 (dd, J=7.5, 0.8 Hz, 1H), 7.63-7.59 (m, 1H), 7.52 (t, J=7.6 Hz, 1H), 7.46 (d, J=8.0 Hz, 2H), 5.16 (dd, J=13.3, 5.1 Hz, 1H), 4.37 (dt, J=41.1, 17.7 Hz, 3H), 3.60 (t, J=6.6 Hz, 2H), 3.44 (t, J=6.5 Hz, 2H), 3.19 (s, 2H), 2.91 (d, J=12.3 Hz, 1H), 2.70 (t, J=6.6 Hz, 2H), 2.59 (d, J=16.2 Hz, 1H), 2.46-2.37 (m, 4H), 2.05-2.00 (m, 1H), 1.78 (d, J=8.3 Hz, 2H), 1.64-1.43 (m, 6H), 1.36 (d, J=5.1 Hz, 9H).

### Step 6: UB-180961i

Compound V2407-048 (3.6 g, 5.1 mmol) and NaN3 (667 mg, 10.2 mmol) were dissolved in DMF (10 mL) and then reacted at 80° C. overnight. The reaction solution was extracted twice with EtOAc (100 mL), and the organic phase was dried and separated by column chromatography (PE/EtOAc=0-100%) to afford the target product UB-180961i (2.4 g, 68% yield) as white solid.

¹H NMR (400 MHz, DMSO) δ 11.00 (s, 1H), 7.72 (d, J=7.4 Hz, 1H), 7.63 (d, J=7.3 Hz, 1H), 7.52 (t, J=7.6 Hz, 1H), 5.15 (dd, J=13.3, 5.1 Hz, 1H), 4.44 (d, J=17.7 Hz, 1H), 4.31 (d, J=17.7 Hz, 1H), 3.92 (s, 1H), 3.63 (t, J=6.6 Hz, 2H), 3.49 (t, J=6.3 Hz, 2H), 3.24 (s, 2H), 3.00-2.85 (m, 1H), 2.73 (t, J=6.6 Hz, 2H), 2.61 (s, 1H), 2.46-2.37 (m, 1H), 2.02 (d, J=5.5 Hz, 1H), 1.93-1.44 (m, 8H), 1.38 (s, 10H).

### Step 7: UB-180961

Synthesised using a method similar to General Method 2. LCMS [M+H]⁺=884.6; ¹H NMR (400 MHz, DMSO-d₆) δ 13.08 (s, 1H), 11.01 (s, 1H), 9.62 (s, 1H), 8.96 (m, 3H), 7.76-7.68 (m, 3H), 7.66-7.61 (m, 2H), 7.56-7.49 (m, 2H), 5.16 (dd, J=13.3, 5.1 Hz, 1H), 4.71 (m, 1H), 4.51-4.44 (m, 2H), 4.32 (d, J=17.8 Hz, 1H), 3.91 (s, 3H), 3.80 (t, J=5.3 Hz, 2H), 3.69 (t, J=6.7 Hz, 2H), 3.32 (m, 1H), 3.21 (s, 3H), 3.16 (m, 2H), 2.97-2.89 (m, 1H), 2.79 (t, J=6.7 Hz, 2H), 2.59 (m, 1H), 2.44 (m, 1H), 2.05-1.73 (m, 14H), 1.46 (m, 2H), 1.41-1.33 (m, 2H), 0.75 (t, J=7.4 Hz, 3H).

### Synthesis of Compound UB-181103

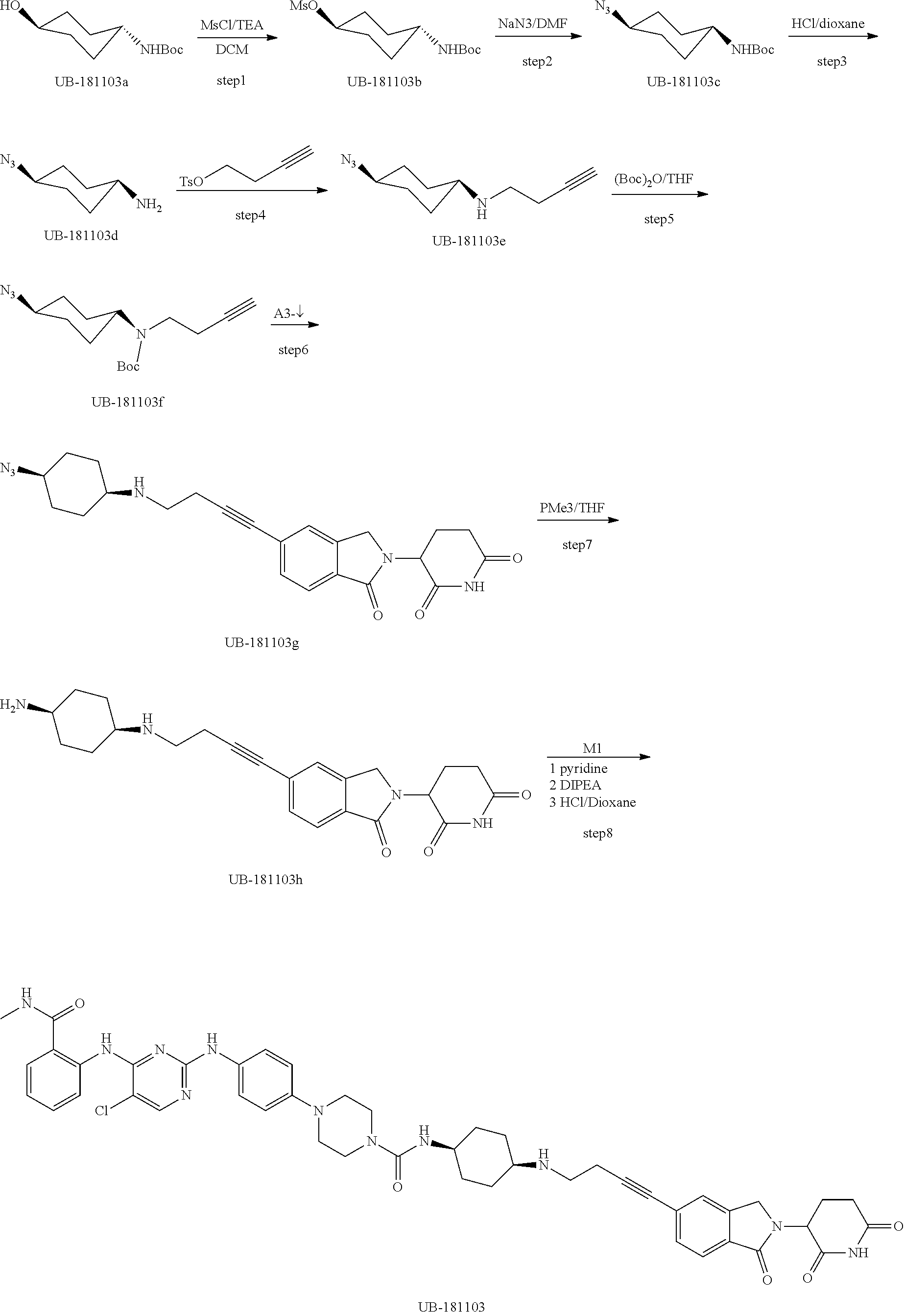

### Step 1: UB-181103b (V2714-018)

To a solution of UB-181103a (10 g, 22 mmol) and triethylamine (7.05 g, 70 mmol) in dichloromethane (10 ml) was added dropwise methanesulfonyl chloride (6.89 g, 60 mmol), and the mixture was stirred overnight at room temperature. After completion of reaction, the mixture was added with water (10 ml) and extracted with DCM (10 ml*3). The organic layer was dried over Na₂SO₄ and concentrated to obtain UB-181103b as white solid (13g, yield: 98%.) LCMS [M+H]⁺=294.3

### Step 2: UB-181103c (V2714-019)

UB-181103b (13 g, 44 mmol) was mixed with sodium azide (3.75 g, 58 mmol) and dissolved in DMF (10 ml). The mixture was stirred at room temperature overnight. After completion of the reaction, the reaction solution was diluted with H₂O (300 ml) and extracted with ether (2×150 ml). The organic phase was washed with H₂O (3×100 ml) and saline (1×100 ml), dried over MgSO₄, filtered, and the solvent was removed under low pressure to obtain product UB-181103c (9 g, yield: 88%) LCMS [M+H]⁺=241.3

### Step 3: UB-181103d (V2714-020)

Compound UB-181103c (10 g, 0.042 mmol) and hydrochloric acid in dioxane (100 mL, 4 N) were added to tetrahydrofuran (10 mL) and reacted at room temperature for 2 h. Upon completion of the reaction, the reaction was concentrated by rotary evaporation under reduced pressure to give compound UB-181103d (5.8 g, 99% yield). LCMS [M+H]⁺=141.3

### Step 4: UB-181103e (V2714-027)

Compound UB-181103d (1.0 g, 5.68 mmol), 3-butynyl p-toluenesulfonate (1.27 g, 5.68 mmol) and triethylamine (6.06 g, 60 mmol) were mixed and then dissolved in toluene (20 mL). The solution was reacted at 80° C. for 18 hours. Upon completion of the reaction, the reaction was filtrated and the filtrate was concentrated by rotary evaporation under reduced pressure, and isolated by silica gel column chromatography (DCM/MeOH=10/1) to obtain UB-181103e (818 mg, yield 75%) as colorless oil. LCMS [M+H]⁺=193.3

### Step 5: UB-181103f (V2714-032)

Compound UB-181103e (350 mg, 1.82 mmol), di-tert-butyl dicarbonate (441 mg, 2.03 mmol) and sodium bicarbonate (360 mg, 4.29 mmol) were added to tetrahydrofuran (20 mL) successively and reacted at room temperature for 2 hours. Upon completion of the reaction, the reaction was poured into 10 mL of water and extracted with dichloromethane (5 mL*3). The organic phases were combined, then washed with saturated saline, dried over anhydrous Na₂SO₄, and concentrated by rotary evaporation under reduced pressure to obtain compound UB-181103f (463 mg, yield 87%) as colorless oil. LCMS [M+H]⁺=293.3

### Step 6: UB-181103g (V2714-033)

Compound UB-181103f (30 mg, 0.103 mmol) and A3-I (38 mg, 0.103 mmol) were dissolved in DMF (10 mL), and then added with dichlorobis(triphenylphosphonium)palladium (7.2 mg, 0.010 mmol), cuprous iodide (3.91 mg, 0.021 mmol) and triethylamine (150 mg, 1.49 mmol). The mixture was reacted at 80° C. overnight. The reaction solution was filtered through diatomite and the filtrate was concentrated to obtain the crude product, which was purified by rapid chromatography (elution with DCM/MeOH=0%-20% for 30 min) to give the product UB-181103g (9 mg, 17% yield). LCMS [M+H]⁺=435.5

### Step 7: UB-181103h (V2714-034)

UB-181103g (1 g, 1.87 mmol) was dissolved in THF (10 mL), and then added with trimethylphosphine (402 mg, 1.87 mmol). The mixture was reacted at room temperature overnight. Upon completion of the reaction, the reaction solution was concentrated to obtain the crude product, which was purified by rapid chromatography (DCM/MeOH=10/1) to obtain compound UB-181103h (510 mg, yield 91%). LCMS [M+H]⁺=409.5

### Step 8: UB-181103 (V2714-035)

Synthesised using a method similar to General Method 6. ¹H NMR (400 MHz, DMSO-d₆) δ11.85 (s, 1H), 11.00 (s, 1H), 9.78 (s, 1H), 9.16 (s, 2H), 8.84 (d, J=4.5 Hz, 1H), 8.69 (s, 1H), 8.27 (s, 1H), 7.80 (d, J=6.8 Hz, 1H), 7.70 (dd, J=8.3, 2.3 Hz, 2H), 7.56 (dt, J=15.9, 9.9 Hz, 3H), 7.20 (dd, J=18.0, 10.0 Hz, 1H), 6.20 (s, 1H), 5.11 (dd, J=13.3, 5.0 Hz, 1H), 4.46 (d, J=7.7 Hz, 1H), 4.33 (d, J=7.7 Hz, 1H), 3.80 (d, J=7.9 Hz, 4H), 3.31 (t, J=6.2 Hz, 4H), 3.30-3.15 (m, 4H), 2.98-2.76 (m, 3H), 2.67 (dd, J=3.4, 5.7 Hz, 3H), 2.65-2.51 (m, 2H), 2.45-2.31 (m, 2H), 2.05-1.97 (m, 1H), 1.93-1.77 (m, 4H), 1.70-1.59 (m, 2H). LCMS [M+H]⁺=872.9

### Synthesis of Compound UB-181189

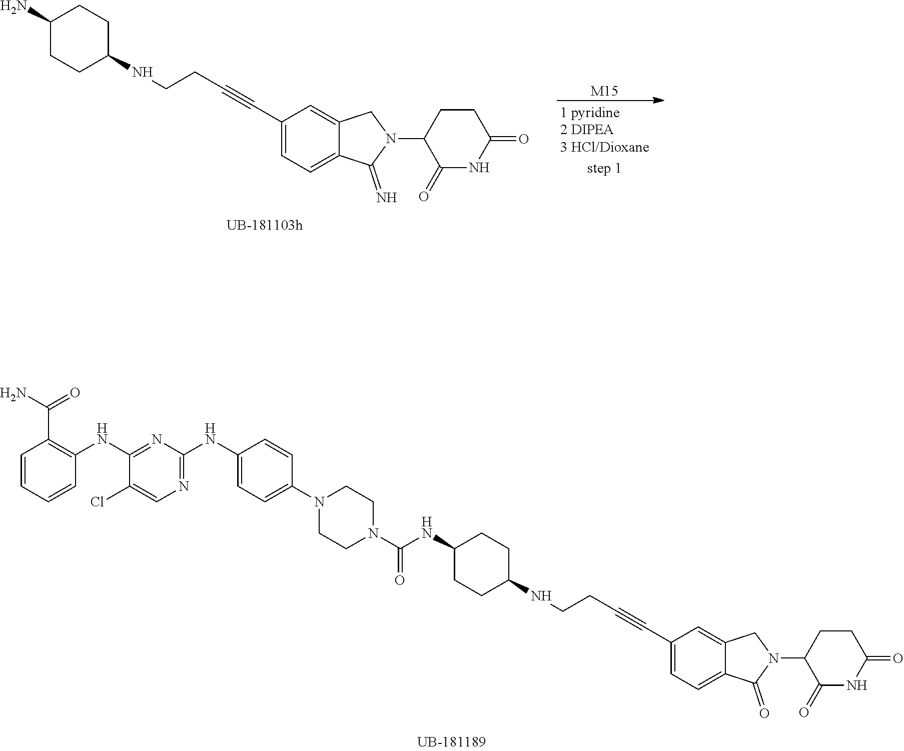 [SMILES: N=C1c2ccc(C#CCCN[C@H]3CC[C@@H](N)CC3)cc2CN1C1CCC(=O)NC1=O ; "step 1" ; "M15" ; NC(=O)c1ccccc1Nc1nc(Nc2ccc(N3CCN(C(=O)N[C@H]4CC[C@@H](NCCC#Cc5ccc6c(c5)CN(C5CCC(=O)NC5=O)C6=O)CC4)CC3)cc2)ncc1Cl]

### Step 1: UB-181189

Synthesised using a method similar to General Method 6. LCMS [M+H]⁺=859.4.

### Synthesis of Compound M17

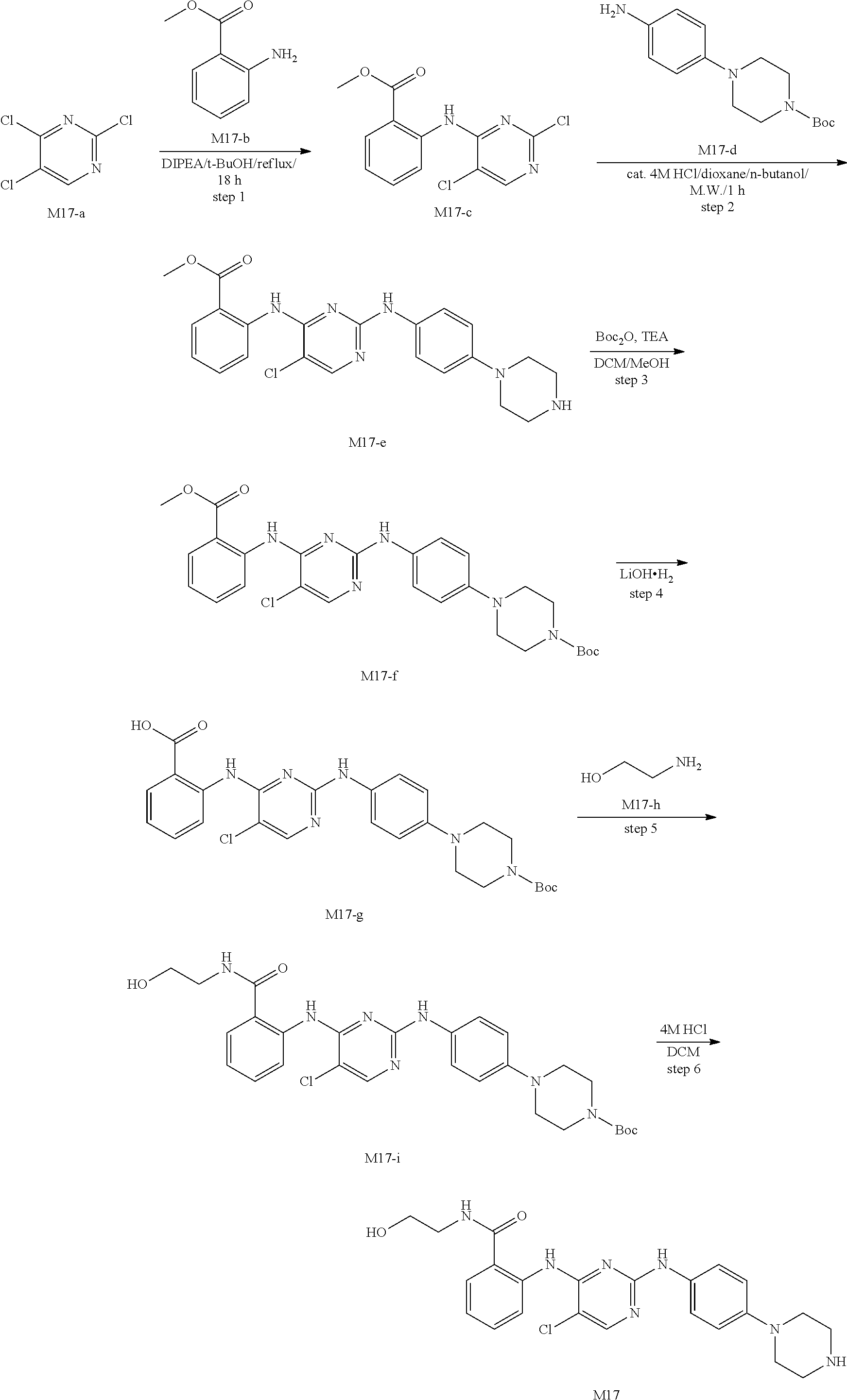

### Step 1: M17-c

To a solution of M17-a (2 g, 13.2 mmol) in t-BuOH (30 mL) was added M17-b (2.4 g, 13.2 mmol) and 3.1 ml of DIPEA, and then the mixture was stirred at 90° C. for 18 hours. The mixture was concentrated in vacuo to obtain solid. After the addition of ether, the mixture was sonicated for 10 min, then filtered to give M17-c (1.8 g, 46% yield) as white solid. LCMS [M+H]⁺=298.1

### Step 2: M17-e

To a solution of M17-c (300 mg, 1.0 mmol) in n-BuOH (7 mL) was added M17-d (279 mg, 1.0 mmol), and then the mixture was added with HCl (0.5 mL), and stirred and reacted for 1 h at 150° C. via microwave under N2 protection. The reaction mixture was added to ether and filtered to give M17-e (400 mg, 90% yield) as yellow solid. LCMS [M+H]⁺=439.1

### Step 3: M17-f

To a solution of M17-e (480 mg, 1.6 mmol) in DCM/MeOH (20 ml) was added TEA (325 mg, 3.2 mmol) and Boc₂O (702 mg, 3.2 mmol). The mixture was stirred at room temperature for 2 h. The reaction mixture was concentrated to give crude product, which was then subjected to silica gel column (DCM/MeOH) to give M17-f (300 mg, 51% yield) as white solid. LCMS [M+H]⁺=539.4

### Step 4: M17-g

To a solution of M17-f (300 mg, 0.56 mmol) in THF/MeOH/H₂O (100 mL) was added NaOH (111 mg, 2.78 mmol) and then the mixture was stirred at 40° C. for 16 hours. The reaction mixture was concentrated, adjusted to pH 5 with 1 M HCl, extracted with ethyl acetate (200 mL*1) and the organic phase was concentrated to give M17-g (250 mg) as grey solid. LCMS [M+H]⁺=525.5

### Step 5: M17-i

To a solution of M17-g (50 mg, 0.095 mmol) in DMF (2 mL) was added HATU (109 mg, 0.286 mmol) and DIEA (37 mg, 0.286 mmol) and the mixture was then stirred for 2 hours at room temperature. Then M17-h (5.8 mg, 0.095 mmol) was added to the mixture and the mixture was stirred continuously at room temperature for 1 hour. The reaction mixture was concentrated to give crude product, which was purified by preparative TLC to give M17-i (40 mg, 74% yield) as white solid. LCMS [M+H]⁺=568.6

### Step 6: M17

To a solution of M17-i (30 mg, 0.056 mmol) in DCM (5 ml) was added 4M HCl (1 ml) and the mixture was stirred at room temperature for 1 h. The reaction mixture was added to ether and filtered to give M17 (45 mg) as white solid. LCMS [M+H]⁺=468.4

### Synthesis of Compound M18

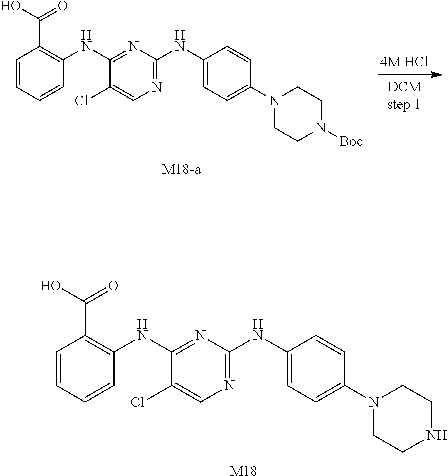 [SMILES: CC(C)(C)OC(=O)N1CCN(c2ccc(Nc3ncc(Cl)c(Nc4ccccc4C(=O)O)n3)cc2)CC1 ; O=C(O)c1ccccc1Nc1nc(Nc2ccc(N3CCNCC3)cc2)ncc1Cl ; "M18"]

### Step 1: M18

To a solution of M18-a (30 mg, 0.056 mmol) in DCM (3 ml) was added 4M HCl (1 ml) and the mixture was stirred at room temperature for 1 h. The reaction mixture was added to ether and filtered to give M18 (25 mg, 90% yield) as white solid. LCMS [M+H]⁺=425.4

### Synthesis of Compound M19

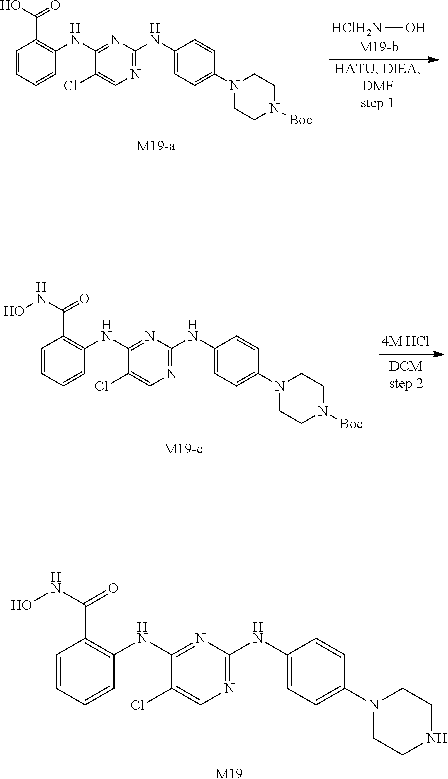

### Step 1: M19-c

To a solution of M19-a (200 mg, 0.381 mmol) in DMF (10 mL) was added HATU (434 mg, 1.14 mmol) and DIEA (147 mg, 1.14 mmol) and the mixture was then stirred for 2 hours at room temperature. Then M19-b (132 mg, 1.91 mmol) was added to the mixture and the mixture was stirred continuously at room temperature for 1 hour. The reaction mixture was concentrated, and then purified by silica gel chromatography to give M19-c (30 mg, 15% yield) as yellow solid. LCMS [M+H]⁺=540.6

### Step 2: M19

To a solution of M19-c (30 mg, 0.056 mmol) in DCM (5 ml) was added 4M HCl (1 ml) and then stirred at room temperature for 1 h. The reaction mixture was added to ether and filtered to give M19 (25 mg, 90% yield) as white solid. LCMS [M+H]⁺=440.5

### Synthesis of Compound M23

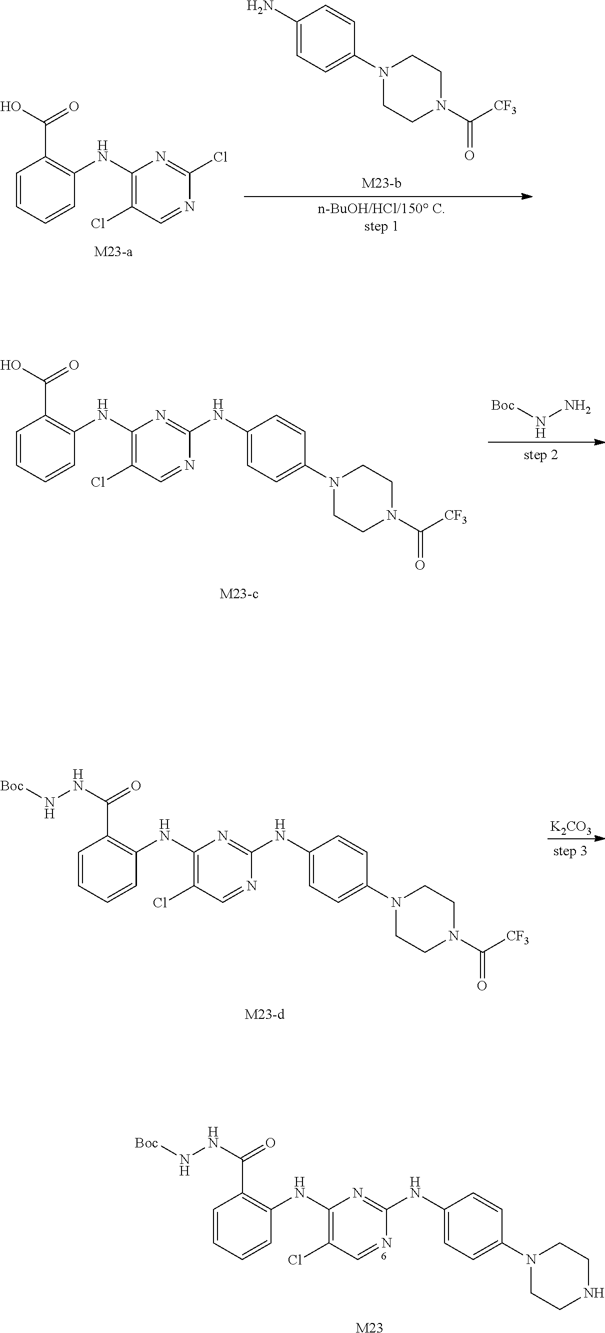

### Step 1: M23-c

Compounds M23-a (70 mg, 0.25 mmol), and M23-b (68 mg, 0.25 mmol) were dissolved in n-butanol (2 mL), followed by the addition of a catalytic amount of 4M HCl in dioxane and heated to 150° C. via microwave for 1 h. The reaction solution was concentrated and then separated by column chromatography (MeOH/DCM=1/10) to give target product M23-c (80 mg, 62.3% yield) as brown solid. LCMS [M+H]⁺=521.2

### Step 2: M23-d

M23-c (40 mg, 0.07 mmol), HATU (44 mg, 0.11 mmol), and DIEA (29 mg, 0.23 mmol) was added to DMF (1 mL) and the mixture was stirred for 2 hours at room temperature. Then BOC-hydrazine (15 mg, 0.1 mol) was added and the reaction system was stirred at room temperature for 1 hour. The reaction mixture was concentrated to give crude product, which was purified by preparative plate to give M23-d (40 mg, 82% yield) as white solid. LCMS [M+H]⁺=635.2

### Step 3: M23

K₂CO₃ (100 mg) was added to a solution of M23-d (30 mg, 0.05 mmol) in MeOH (2 mL) and the reaction was stirred for 16 h at room temperature. The reaction mixture was filtered and concentrated to give yellow M23 (15 mg, 58.9% yield). LCMS [M+H]⁺=539.2

### Synthesis of Compound M24

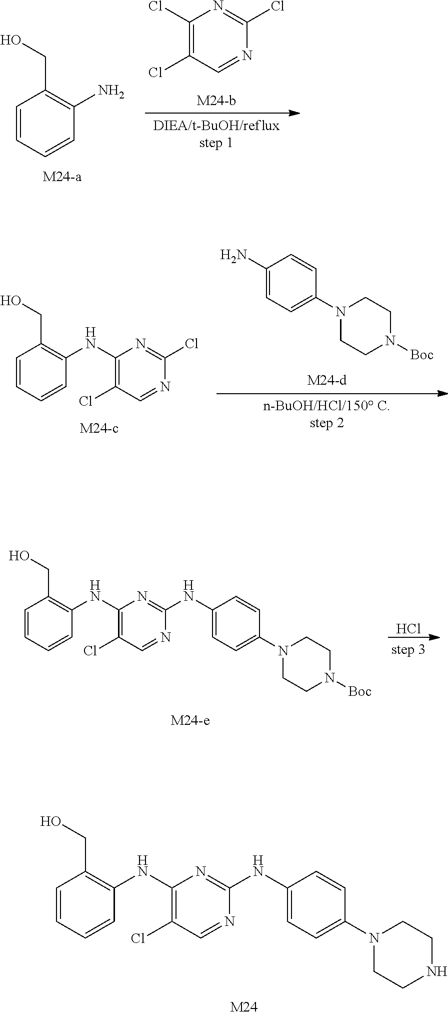

### Step 1: M24-c

Compounds M24-a (50 mg, 0.18 mmol), and M24-b (66.25 mg, 0.18 mmol) were dissolved in n-butanol (12 mL), followed by the addition of DIPEA (0.1 mL) and then heated to 150° C. via microwave synthesizer for 2 h. Upon concentration of the reaction solution, ether was added, and the mixture was sonicated for 10 min, filtered to give M24-c (100 mg, 91.2% yield) as white solid.

¹H NMR (400 MHz, DMSO-d6) δ 9.63 (s, 1H), 8.39 (s, 1H), 7.70 (dd, J=8.0, 1.2 Hz, 1H), 7.43 (dd, J=7.6, 1.6 Hz, 1H), 7.35 (td, J=7.7, 1.7 Hz, 1H), 7.24 (td, J=7.5, 1.3 Hz, 1H), 5.67 (t, J=5.2 Hz, 1H), 4.55 (d, J=5.1 Hz, 2H). LCMS [M+H]⁺=298.1

### Step 2: M24-e

Compounds M24-c (100 mg, 0.37 mmol), and M24-d (102.9 mg, 0.37 mmol) were dissolved in n-butanol (5 mL), followed by the addition of 4M HCl in dioxane (0.1 mL) and then heated to 150° C. via microwave synthesizer for 1 h. The reaction solution was concentrated to obtain M24-c M24-e as yellow solid (70 mg, yield 37% LCMS [M+H]⁺=511.2

### Step 3: M24-f

Compound M24-e (39 mg, 0.08 mol) and hydrochloric acid in dioxane (1 mL, 4 N) were added to dichloromethane (5 mL) and reacted at room temperature for 1 h. Upon completion of the reaction, the reaction was concentrated by rotary evaporation under reduced pressure to give compound M24 (20 mg, 27% yield).

LCMS [M+H]⁺=411.2

### Synthesis of Compound M25

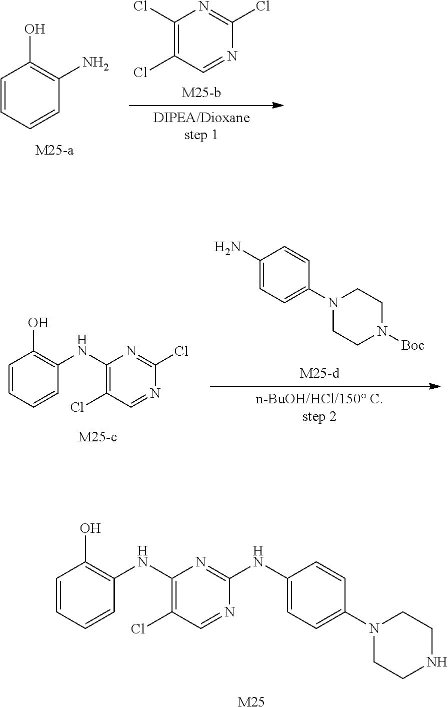 [SMILES: Nc1ccccc1O ; Clc1ncc(Cl)c(Cl)n1 ; Oc1ccccc1Nc1nc(Cl)ncc1Cl ; CC(C)(C)OC(=O)N1CCN(c2ccc(N)cc2)CC1 ; Oc1ccccc1Nc1nc(Nc2ccc(N3CCNCC3)cc2)ncc1Cl ; "M25"]

### Step 1: M25-c

Compounds M24-a M25-a (1500 mg, 8.29 mmol), and M25-b (903 mg, 8.29 mmol) were dissolved in n-butanol (30 mL), followed by the addition of DIPEA (3 mL) and the reaction system was heated to 90° C. overnight. The reaction solution was concentrated and then separated by silica gel chromatography (DCM/MeOH=10/1) to give M25-c (1.1 g, 31.2% yield) as yellow solid.

¹H NMR (400 MHz, DMSO-d6) δ 9.93 (s, 1H), 8.84 (s, 1H), 8.35 (s, 1H), 7.59 (dd, J=7.9, 1.7 Hz, 1H), 7.08 (dd, J=7.8, 1.7 Hz, 1H), 6.94 (dd, J=8.1, 1.4 Hz, 1H), 6.86 (d, J=1.5 Hz, 1H). LCMS [M+H]⁺=255.2

### Step 2: M25

Compounds M25-c (100 mg, 0.37 mmol), M25-d (103 mg, 0.37 mmol) and 4 N hydrochloric acid in dioxane (0.1 mL) were added to n-butanol (4 mL), and the mixture was reacted at 150° C. via microwave for 1 h. Upon completion of the reaction, the reaction was concentrated by rotary evaporation under reduced pressure to give M25 (80 mg, 51.6% yield) as yellow solid. LCMS [M+H]⁺=397.4

### Synthesis of Compound UB-181235

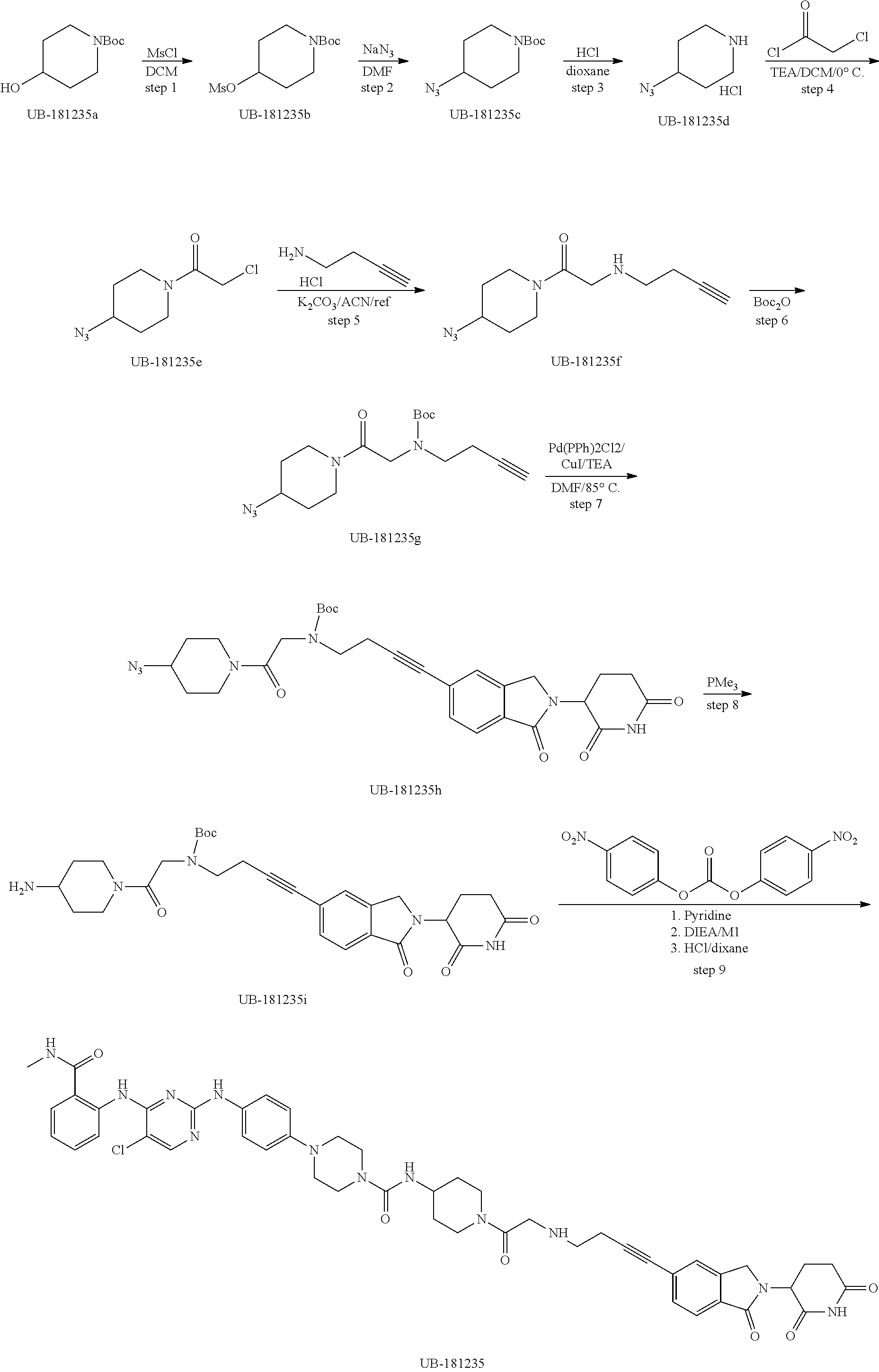

### Step 1: UB-181235a

Compound UB-181235a (10 g, 50 mmol) was dissolved in dichloromethane (100 mL), methanesulfonyl chloride (6.89 g, 60 mmol) and triethylamine (7.05 g, 70 mmol) were added, and the mixture was reacted at 25° C. for 1 h. Upon completion of the reaction, the reaction was poured with 10 mL of water, and extracted with dichloromethane (10 mL*3). The organic phases were combined, then washed with saturated saline, dried over anhydrous Na₂SO₄, concentrated by rotary evaporation under reduced pressure to obtain crude product. The crude product was purified by rapid column chromatography (DCM/MeOH=10/1) to obtain UB-181235b as colorless oil (13g, yield: 94%). LCMS [M+H]⁺=280.3

### Step 2: UB-181235c

UB-181235b (13 g, 47 mmol) was dissolved in DMF (100 mL), and added with sodium azide (3.75 g, 58 mmol). The mixture was stirred at 85° C. under N2 overnight. Upon completion of the reaction, the reaction solution was filtered and concentrated to obtain crude product, which was purified by silica gel column chromatography (DCM/MeOH=30/1) to obtain UB-181235c (9 g, yield 86%) as colorless oil. LCMS [M+H]⁺=227.3

### Step 3: UB-181235d

Compound UB-181235c (10 g, 0.042 mmol) and hydrochloric acid in dioxane (100 mL, 4 N) were added to tetrahydrofuran (10 mL) and reacted at room temperature for 12 h. Upon completion of the reaction, the reaction was concentrated by rotary evaporation under reduced pressure to give compound UB-181235d (5.6 g, 100% yield). LC-MS: [M+H]⁺=127.3

### Step 4: UB-181235e

Compound UB-181235d (1.0 g, 5.68 mmol), 2-chloroacetyl chloride (1.27 g, 5.68 mmol) and triethylamine (6.06 g, 60 mmol) were added to dichloromethane (15 ml) and the mixture was stirred for 18 hours at 30° C. The crude product was purified by rapid column chromatography (PE/ethyl acetate=50% to 100% for 20 min, then MeOH/DCM=0% to 10% for 40 min) to obtain Compound UB-181235e (818 mg, yield 52%) as colorless oil. LCMS [M+H]+=203.6

### Step 5: UB-181235f

UB-181235e (0.72 g, 3.56 mmol), n-butynylamine (0.37 g, 5.34 mmol) and potassium carbonate (1.38 g, 10 mmol) were added to toluene (15 mL) and the mixture was stirred for 18 h at 30° C. Upon completion of the reaction, the reaction was purified by rapid column chromatography (petroleum ether/ethyl acetate=50% to 100% for 20 min, then MeOH/DCM=0% to 10% for 40 min) to obtain Compound UB-181235f (464 mg, yield 75%) as colorless oil. LCMS [M+H]⁺=236.2

### Step 6: UB-181235g

UB-181235f (350 mg, 1.49 mmol) and di-tert-butyl dicarbonate (441 mg, 2.03 mmol) were added to dioxane (13 mL) and the mixture was stirred for 2 hours at room temperature. Upon completion of the reaction, the reaction was added with water (15 mL), and extracted with ethyl acetate (10 mL*3). The crude product obtained by concentration was dissolved in MeOH/DCM=10/1 (30 mL), and the mixture was filtered and concentrated to give compound UB-181235g (434 mg, 87% yield) as colorless oil. LCMS [M+H]+=336.2

### Step 7: UB-181235g

UB-181235f (30 mg, 0.09 mmol) and 3-(5-iodo-1-oxoisoindol-2-yl)piperidine-2,6-dione (38 mg, 0.103 mmol) were dissolved in anhydrous DMF (10 mL), and the mixture was added with Pd(PPh3)₂Cl₂ (7.2 mg, 0.010 mmol) and CuI (3.91 mg, 0.021 mmol) and reacted at 90° C. for 1 h under nitrogen protection. The reaction solution was filtered, concentrated in vacuo to obtain crude product, which was purified by preparative TLC (DCM/MeOH=10/1) to obtain product UB-181235g (9 mg, yield 17%) as white solid. LCMS [M+H]+=578.8

### Step 8: UB-181235h

UB-181235g (1 g, 1.73 mmol) was dissolved in THF (10 mL), PMe₃ (402 mg, 1.87 mmol) was added under N₂ protection, and the mixture was stirred at room temperature overnight. The reaction solution was filtered. The filtrate was concentrated to obtain crude product, which then was purified by silica gel column chromatography (DCM/MeOH=10/1) to obtain product UB-181235h (867 mg, yield 91%) as white solid. LCMS [M+H]+=552.6

### Step 9: UB-181235

Synthesised using a method similar to General Method 6. LCMS [M+H]⁺=915.6; ¹H NMR (400 MHz, DMSO-d₆) δ 11.75 (s, 1H), 11.00 (s, 1H), 9.57 (s, 1H), 9.01-8.77 (m, 2H), 8.71 (d, J=7.4 Hz, 2H), 8.22 (s 1H), 7.82-7.68 (m, 3H), 7.68-7.51 (m, 3H), 7.47 (t, J=7.9 Hz, 1H), 7.17 (dd, J=7.5, 5.4 Hz, 3H), 5.93 (s, 1H), 5.11 (dd, J=13.3, 5.1 Hz, 1H), 4.46 (d, J=7.7 Hz, 1H), 4.33 (d, J=7.7 Hz, 1H), 3.68 (s, 1H), 3.19 (d, J=7.7 Hz, 3H), 3.21-2.90 (m, 3H), 2.90-2.49 (m, 7H), 2.37 (ddd, J=6.2, 10.5, 7.3 Hz, 2H), 2.04-1.99 (m, 2H), 1.83 (dd, J=2.7, 9.1 Hz, 1H), 1.65 (d, J=4.4 Hz, 8H), 1.57-1.27 (m, 4H).

### Synthesis of Compound UB-181236

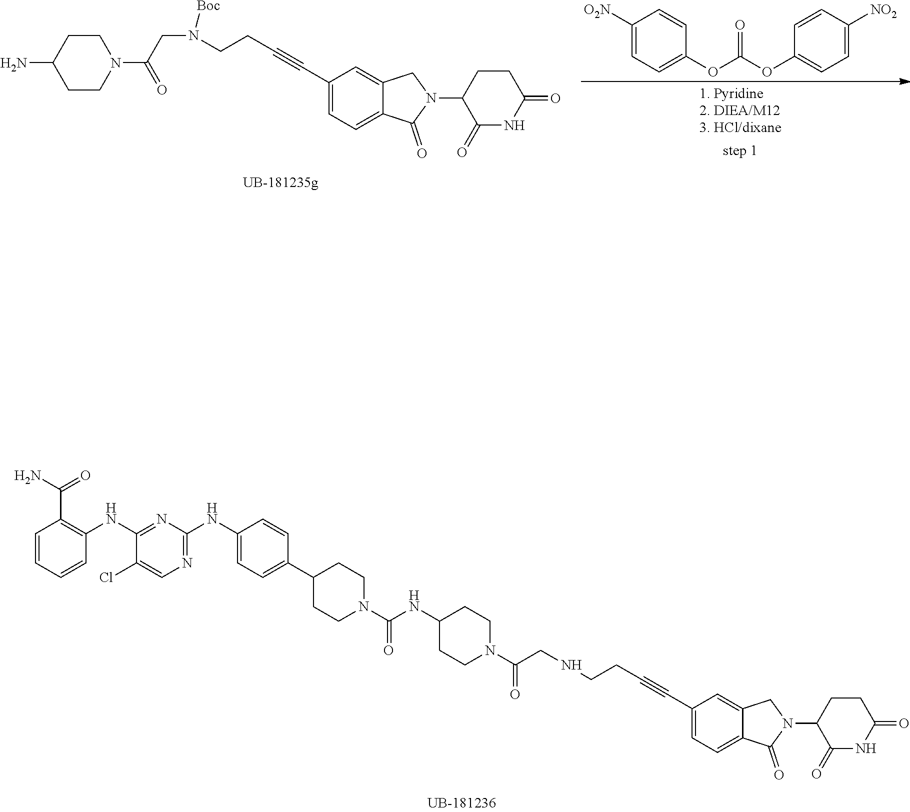 [SMILES: CC(C)(C)OC(=O)N(CCC#Cc1ccc2c(c1)CN(C1CCC(=O)NC1=O)C2=O)CC(=O)N1CCC(N)CC1 ; O=C(Oc1ccc([N+](=O)[O-])cc1)Oc1ccc([N+](=O)[O-])cc1 ; NC(=O)c1ccccc1Nc1nc(Nc2ccc(C3CCN(C(=O)NC4CCN(C(=O)CNCCC#Cc5ccc6c(c5)CN(C5CCC(=O)NC5=O)C6=O)CC4)CC3)cc2)ncc1Cl]

### Step 1: UB-181236

Synthesised using a method similar to General Method 6. ¹H NMR (400 MHz, DMSO-d₆) δ 11.62 (s, 1H), 11.00 (s, 1H), 10.24-10.19 (m, 1H), 9.24 (s, 1H), 8.80 (s, 1H), 8.16 (s, 1H), 7.82-7.65 (m, 3H), 7.59 (t, J=13.4 Hz, 1H), 7.48 (dd, J=14.1, 8.3 Hz, 3H), 6.92 (d, J=9.0 Hz, 2H), 6.48 (s, 1H), 5.12 (dd, J=13.2, 5.0 Hz, 1H), 4.46 (d, J=17.7 Hz, 2H), 4.39-4.28 (m, 1H), 4.28-3.86 (m, 1H), 3.69 (d, J=13.1 Hz, 2H), 3.23 (d, J=8.2 Hz, 4H), 3.18-2.92 (m, 7H), 2.92-2.85 (m, 3H), 2.85-2.47 (m, 6H), 1.99 (dd, J=12.3, 10.1 Hz, 1H), 1.81 (s, 2H), 1.20 (t, J=7.3 Hz, 3H). LCMS [M+H]⁺=901.7

### Synthesis of Compound UB-181239

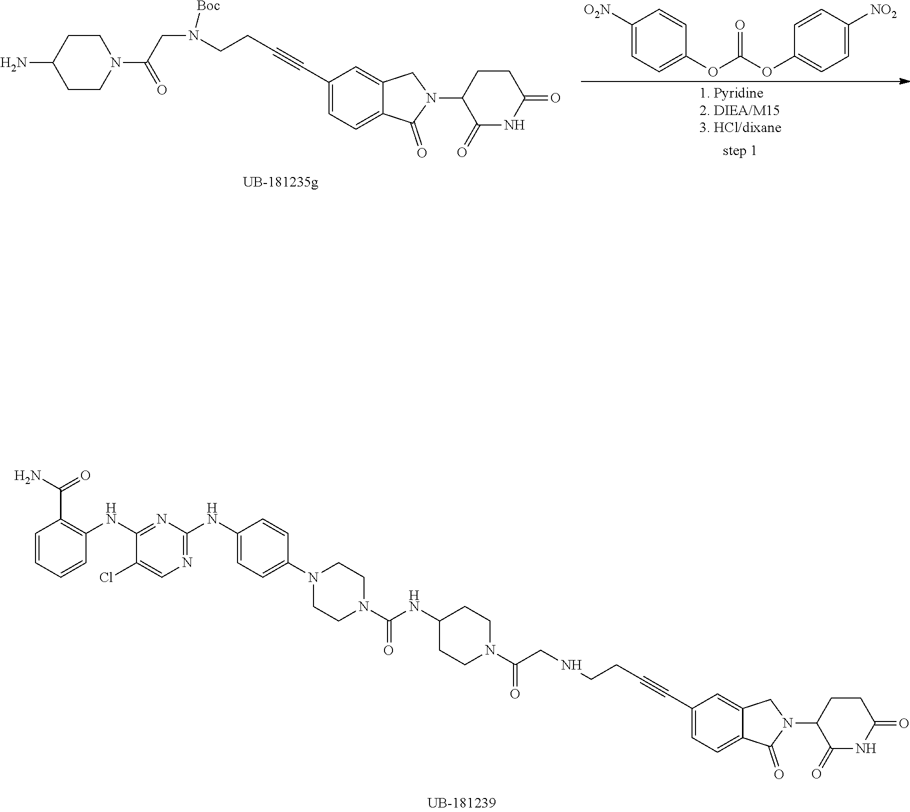 [SMILES: CC(C)(C)OC(=O)N(CCC#Cc1ccc2c(c1)CN(C1CCC(=O)NC1=O)C2=O)CC(=O)N1CCC(N)CC1 ; O=C(Oc1ccc([N+](=O)[O-])cc1)Oc1ccc([N+](=O)[O-])cc1 ; NC(=O)c1ccccc1Nc1nc(Nc2ccc(N3CCN(C(=O)NC4CCN(C(=O)CNCCC#Cc5ccc6c(c5)CN(C5CCC(=O)NC5=O)C6=O)CC4)CC3)cc2)ncc1Cl]

### Step 1: UB-181239

Synthesised using a method similar to General Method 6. LCMS [M+H]⁺=901.8; ¹H NMR (400 MHz, DMSO-d₆) δ 11.87 (s, 1H), 11.00 (s, 1H), 10.28 (s, 2H), 9.24 (s, 1H), 8.80 (s, 1H), 8.46 (s, 1H), 8.16 (s, 1H), 7.87-7.66 (m, 5H), 7.48 (t, J=10.2 Hz, 3H), 7.09 (s, 1H), 6.93 (d, J=9.1 Hz, 2H), 6.58-6.43 (m, 1H), 5.12 (dd, J=13.2, 5.0 Hz, 1H), 4.39-4.23 (m, 3H), 4.07 (dd, J=3.48, 1.66 Hz, 2H), 3.73-3.49 (m, 4H), 3.46 (s, 2H), 3.05 (q, J=7.3 Hz, 44H), 2.92 (dd, J=5.9, 11.8 Hz, 2H), 2.46-2.25 (m, 1H), 1.99 (dd, J=12.3, 10.1 Hz, 1H), 1.78 (d, J=22.7 Hz, 2H), 1.46-1.27 (m, 2H), 1.20 (t, J=7.3 Hz, 3H), 1.03 (t, J=7.3 Hz, 1H).

### Synthesis of Compound UB-181240

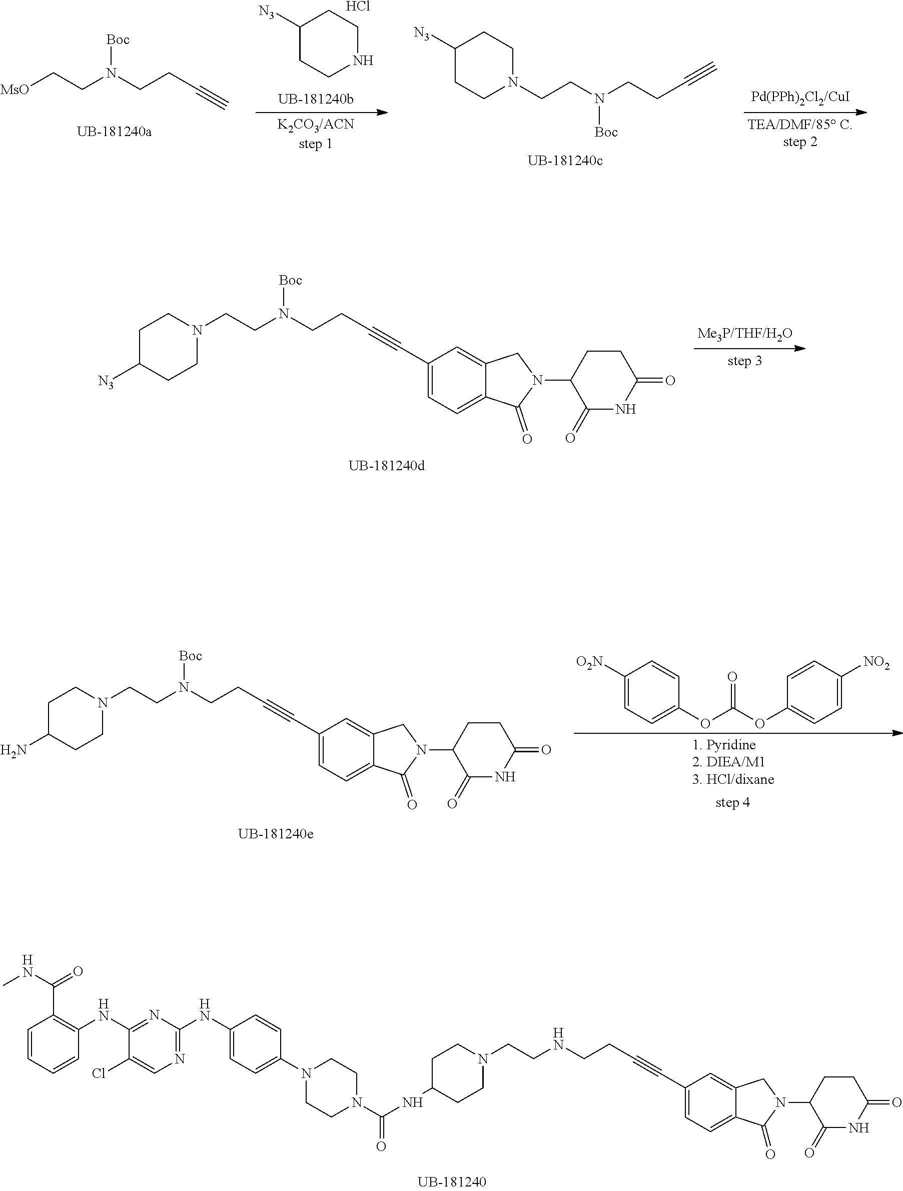

### Step 1: UB-181240c

Compound UB-181240a (400 mg, 1.37 mmol) was dissolved in ACN 10 mL), and added with UB-181240b (222 mg, 1.37 mmol), K₂CO₃ (569 mg, 4.12 mmol) and reacted at 80° C. overnight. The reaction solution was filtered after cooling. The reaction solution was concentrated, and the crude product was separated by column chromatography (PE/EtOAc=0˜10%) to afford product UB-181240c (180 mg, 40.8% yield) as colorless oil. LCMS [M+H]⁺=322.4

¹H NMR (400 MHz, chloroform-d) δ 3.92-3.53 (m, 3H), 3.42 (t, J=6.6 Hz, 2H), 3.32-3.04 (m, 4H), 3.03-2.71 (m, 2H), 2.45 (td, J=6.6, 2.7 Hz, 2H), 2.23 (t, J=7.8 Hz, 2H), 2.09 (s, 2H), 2.04-1.79 (m, 3H), 1.48 (s, 9H).

### Step 2: UB-181240d

A mixture of UB-181240c (50 mg, 0.16 mmol), A3-I (57.6 mg, 0.16 mmol), Pd(PPh₃)₂Cl₂ (6 mg), CuI (3 mg), and TEA (32 mg) was dissolved in dry DMF (5 mL) and reacted at 80° C. for 2 h under N2 protection. The reaction solution was concentrated, and the crude product was separated by column chromatography (DCM/MeOH=0-10%) to give product UB-181240d (35 mg, 40% yield) as yellow oil. LCMS [M+H]⁺=564.3.

### Step 3: UB-181240e

Compound UB-181240d (70 mg, 0.14 mmol) was dissolved in THF (2 mL), and added with water (0.5 mL) and 1M Me₃P (0.5 mL, 0.5 mmol), and the mixture was reacted at room temperature overnight. The reaction solution was concentrated to obtain crude UB-181240e (50 mg, yield 75%) as yellow solid. LCMS [M+H]⁺=538.4.

### Step 4: UB-181240

Synthesised using a method similar to General Method 6. ¹H NMR (400 MHz, DMSO-d6) δ 12.00 (s, 1H), 11.02 (s, 1H), 10.93 (s, 1H), 10.17 (s, 1H), 10.01 (s, 2H), 9.01 (q, J=4.4 Hz, 1H), 8.61 (d, J=8.3 Hz, 1H), 8.32 (s, 1H), 7.85 (d, J=7.9 Hz, 1H), 7.77-7.59 (m, 7H), 7.53 (s, 1H), 7.19 (s, 1H), 6.95 (d, J=20.9 Hz, 1H), 5.16-5.06 (m, 1H), 4.51-4.33 (m, 2H), 3.59 (s, 2H), 3.54-3.48 (m, 4H), 3.43 (s, 4H), 3.25 (s, 5H), 3.01 (d, J=7.2 Hz, 2H), 2.90 (ddd, J=17.9, 13.5, 5.2 Hz, 1H), 2.79 (d, J=4.3 Hz, 3H), 2.62-2.54 (m, 1H), 2.38 (qd, J=13.2, 4.4 Hz, 1H), 2.03-1.81 (m, 5H), 1.33-1.22 (m, 4H). LCMS [M+H]⁺=901.98.

### Synthesis of Compound UB-181249

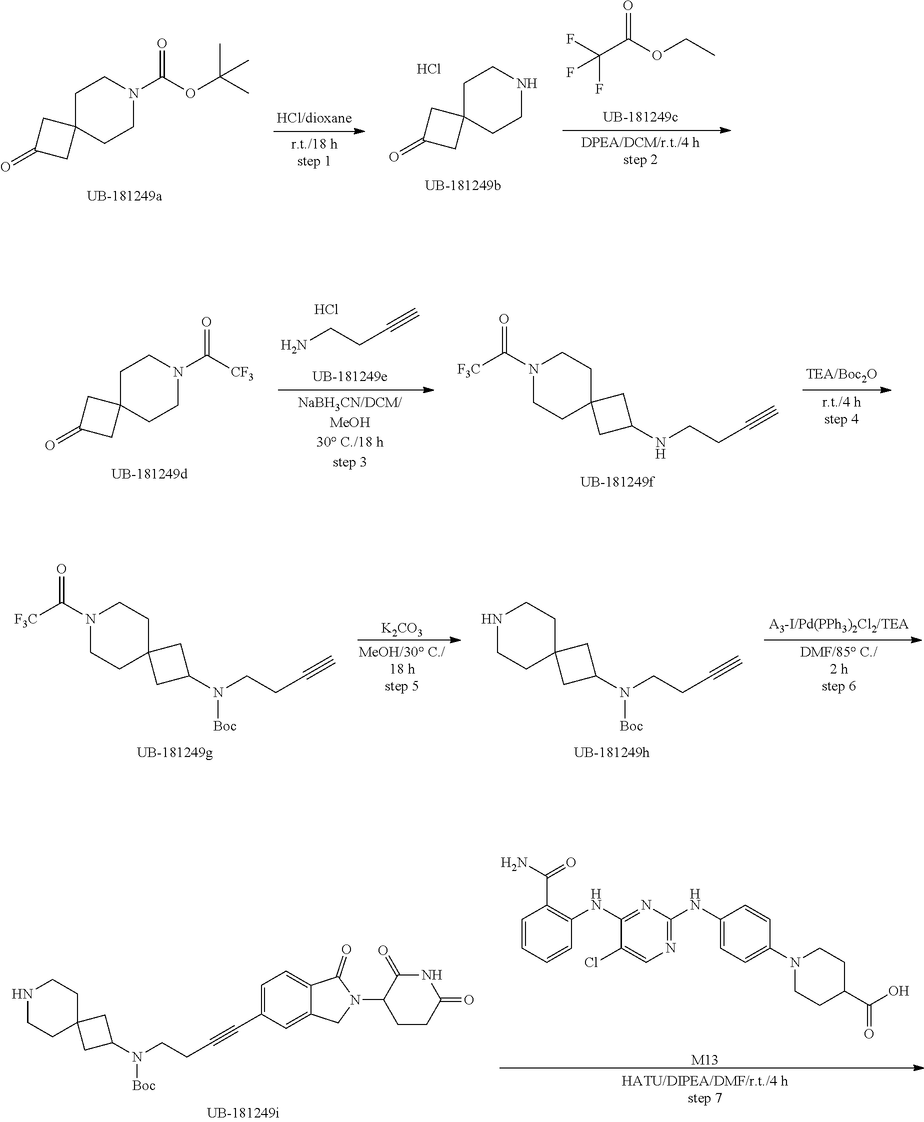

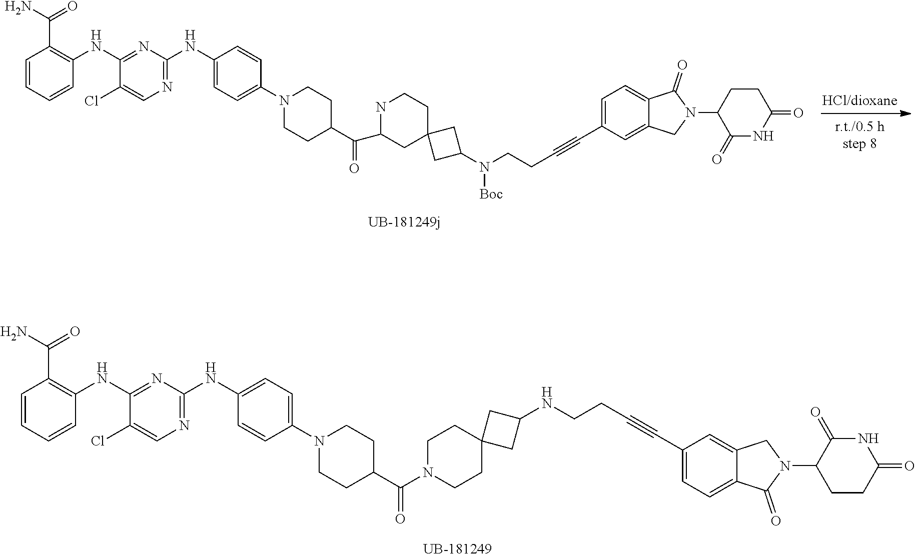 [SMILES: CC(C)(C)OC(=O)N(CCC#Cc1ccc2c(c1)CN(C1CCC(=O)NC1=O)C2=O)C1CC2(CCNC(C(=O)C3CCN(c4ccc(Nc5ncc(Cl)c(Nc6ccccc6C(N)=O)n5)cc4)CC3)C2)C1 ; NC(=O)c1ccccc1Nc1nc(Nc2ccc(N3CCC(C(=O)N4CCC5(CC4)CC(NCCC#Cc4ccc6c(c4)CN(C4CCC(=O)NC4=O)C6=O)C5)CC3)cc2)ncc1Cl]

### Step 1: UB-181249b

Compound UB-181249 (4 g, 16.7 mmol) was dissolved in DCM (15 mL), and added with 4M HCl/dioxane (20 mL, 80 mmol). The mixture was reacted at room temperature overnight. The solvent was spin-dried to obtain crude product UB-181249b (2.9 g, yield 100%) as white solid. LCMS [M+H]⁺=140.6

### Step 2: UB-181249d

Compound UB-181249b (700 mg, 4 mmol) was dissolved in DCM (20 mL), and added with DIPEA (1.4 mL, 8 mmol) and UB-181249c (1.13 g, 8 mmol). The mixture was reacted for 4 h at room temperature. The reaction solution was concentrated, and the crude product was separated by column chromatography (PE/EtOAc=0-80%) to give product UB-181249d (300 mg, 31% yield) as white solid. LCMS [M+H]⁺=236.0.

### Step 3 & 4: UB-181249g

A mixture of UB-181249d (300 mg, 1.27 mmol), and UB-181249e (160 mg, 1.53 mmol) was dissolved in MeOH (5 mL) and DCM (10 mL), and reacted at room temperature for 2 h. NaBH₃CN (160 mg, 2.54 mmol) was added and the mixture was reacted at room temperature overnight. The above reaction solution was added with TEA (0.3 mL) and Boc₂O (0.5 mL), and reacted at room temperature for 4 h. The reaction solution was concentrated and the crude product was separated by column chromatography (DCM/MeOH=0˜10%) to obtain product UB-181249g (100 mg, 20% yield) as yellow oil. LCMS [M+H]⁺=389.4.

### Step 5: UB-181249h

Compound UB-181249g (100 mg, 0.25 mmol) was dissolved in MeOH (15 mL) and added with K₂CO₃ (107 mg, 0.77 mmol), and the mixture was reacted at 30° C. overnight. The reaction solution was filtered to remove insoluble matters. The filtrate was concentrated, and the crude product was separated by column chromatography (DCM/MeOH=0-100%) to give product UB-181249h (50 mg, 68% yield) as yellow oil. LCMS [M+H]⁺=293.3

### Step 6: UB-181249i

A mixture of UB-181249h (50 mg, 0.17 mmol), A3-I (63 mg, 0.17 mmol), Pd(PPh₃)₂Cl₂ (11 mg, 0.017 mmol), CuI (3 mg, 0.017 mmol), and TEA (4.2 ml, 0.17 mmol) were dissolved in dry DMF (4 mL) and reacted at 80° C. overnight under N2 protection. The reaction solution was concentrated and the crude product was separated by column chromatography (DCM/MeOH=0-100%) to give product UB-181249i (20 mg, 22% yield) as brown solid. LCMS [M+H]⁺=535.6

### Step 7: UB-181249j

Compound UB-181249i (20 mg, 0.037 mmol), M13 (18 mg, 0.037 mmol), HATU (76 mg, 0.2 mmol), and DIPEA (0.1 mL) were dissolved in DMF (3 mL), and reacted at room temperature for 4 hours. The reaction solution was concentrated and the crude product was purified by thin layer chromatography (DCM/MeOH=12/1) to obtain product UB-181249j (2 mg, 5.4% yield) as yellow solid. LCMS [M+H]⁺=998

### Step 8: UB-181249

Compound UB-181249j (2 mg, 0.002 mmol) was dissolved in DCM (3 mL) and MeOH (0.5 mL). 4M HCl/dioxane (0.5 mL) was added, and the mixture was reacted at room temperature for 30 minutes. The reaction supernatant was removed and the solid was pulped with Et₂O (10 mL*2). The solid was dried to obtain product UB-181249 (0.6 mg, yield 32%) as yellow solid. LCMS [M/2+H]⁺=443.9.

NMR: NA

### Synthesis of Compound UB-181250

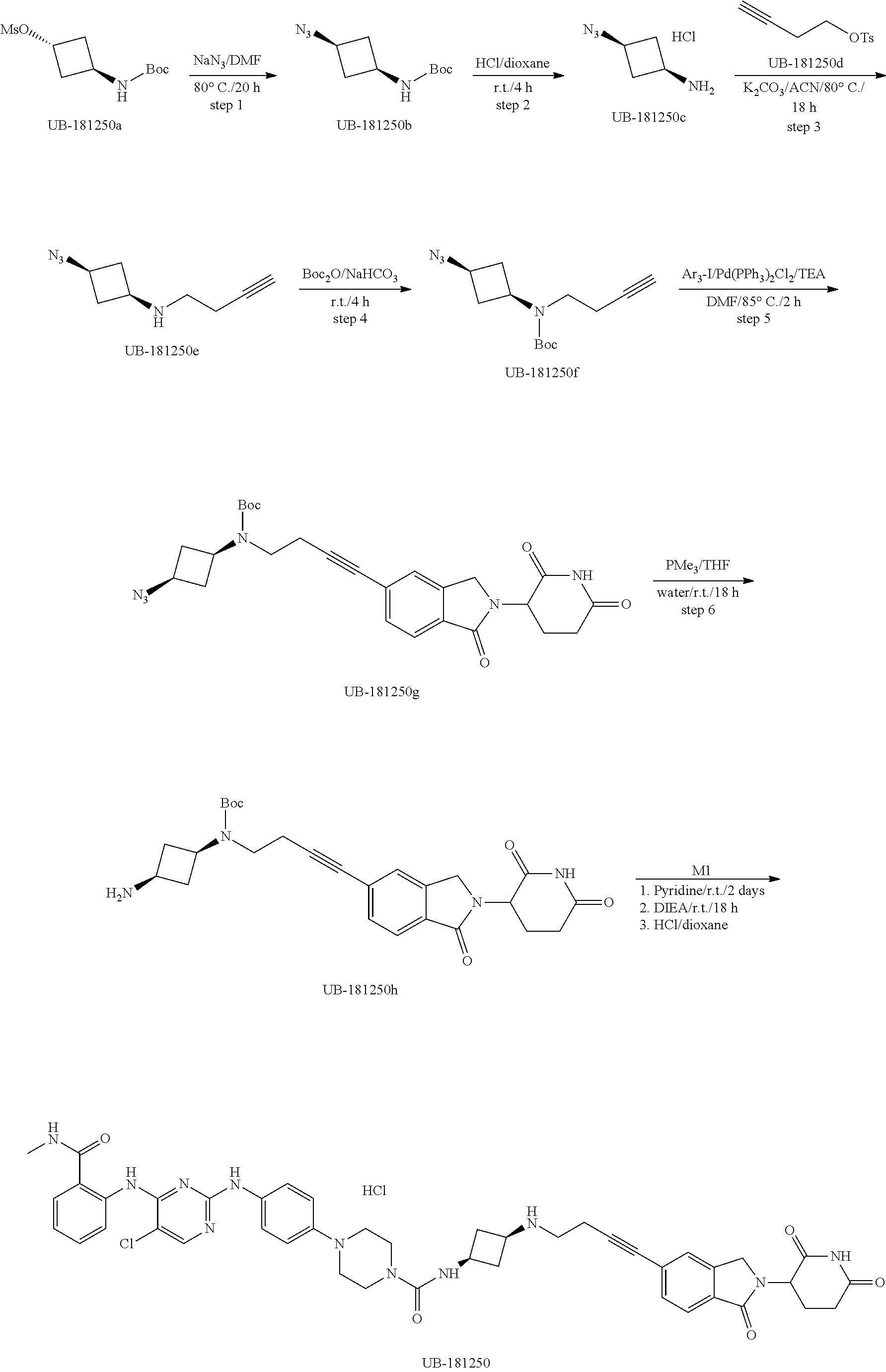

### Step 1: UB-181250b

Compound UB-181250a (3.4 g, 13 mmol) and NaN₃ (1.7 g, 26 mmol) were dissolved in DMF (50 mL), and reacted at 85° C. overnight. The reaction solution was added with saturated saline (20 mL), and then extracted with EtOAc (30 mL*2). The organic phase was washed with water and saline, dried, and concentrated to obtain yellow solid product (2.2 g, yield 80%). LCMS [M+H]⁺=213.2

### Step 2: UB-181250c(3)

Compound UB-181250b (1 g, 4.7 mmol) was dissolved in DCM (10 mL), 4M HCl/dioxane (6 mL, 23.5 mmol) was added, and the mixture was reacted at room temperature for 4 hours. The solvent was spin-dried to obtain crude product UB-181250c (0.7 g, yield 100%) as white solid. LCMS [M+H]⁺=113.1

### Step 3 & 4: UB-181250f

Compound UB-181250c (0.7 g, 2.7 mmol) in ACN (30 mL) was added with UB-181250d (1.2 g, 5.6 mmol), and K₂CO₃ (0.77 g, 5.6 mmol) and reacted at 80° C. overnight. The reaction solution was filtered after cooling. The filtrate was added with aq. NaHCO₃ (1 mL) and Boc₂O (1.5 mL) and reacted for 4 hours at room temperature. The reaction solution was concentrated, and the crude product was separated by column chromatography (PE/EtOAc=0-20%) to give product UB-181250f (300 mg, 25% yield) as colorless oil. LCMS [M+H]⁺=265.3

### Step 5: UB-181250g

A mixture of UB-181250f (75 mg, 0.28 mmol), A1-I (100 mg, 0.28 mmol), Pd(PPh₃)₂Cl₂ (20 mg, 0.028 mmol), CuI (5.3 mg, 0.028 mmol), and TEA (4.2 ml, 0.28 mmol) were dissolved in dry DMF (4 mL) and reacted at 80° C. for 2 hours. The reaction solution was concentrated and the crude product was separated by column chromatography (DCM/MeOH=0-10%) to give product UB-181250g (30 mg, 21% yield) as brown solid. LCMS [M+H]⁺=507.5

### Step 6: UB-181250h

Compound UB-181250g (30 mg, 0.059 mmol) was dissolved in THF (2 mL), and added with 1M Me₃P (0.5 mL, 0.5 mmol), and the mixture was reacted at room temperature for 1 hour. Then water (0.5 mL) was added, and the mixture was reacted overnight at room temperature. The reaction solution was concentrated, and the crude product was purified by thin layer chromatography (EtOAc) to give product UB-181250h (20 mg, 71% yield) as yellow oil. LCMS [M+H]⁺=481.5

### Step 7: UB-181250

Synthesised using a method similar to General Method 6. LCMS [M/2+H]⁺=423.2. NMR: NA

### Synthesis of Compound UB-181251

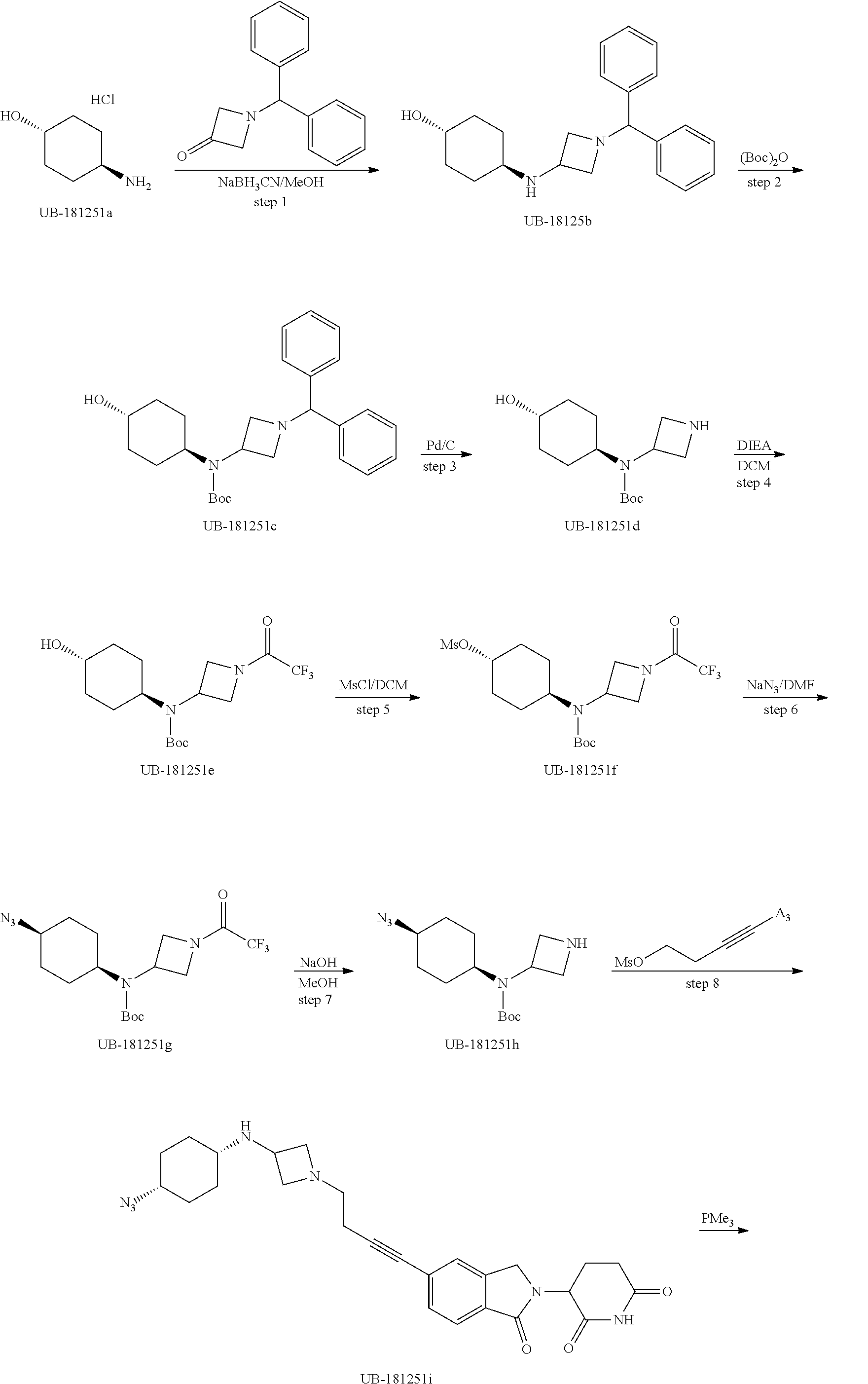

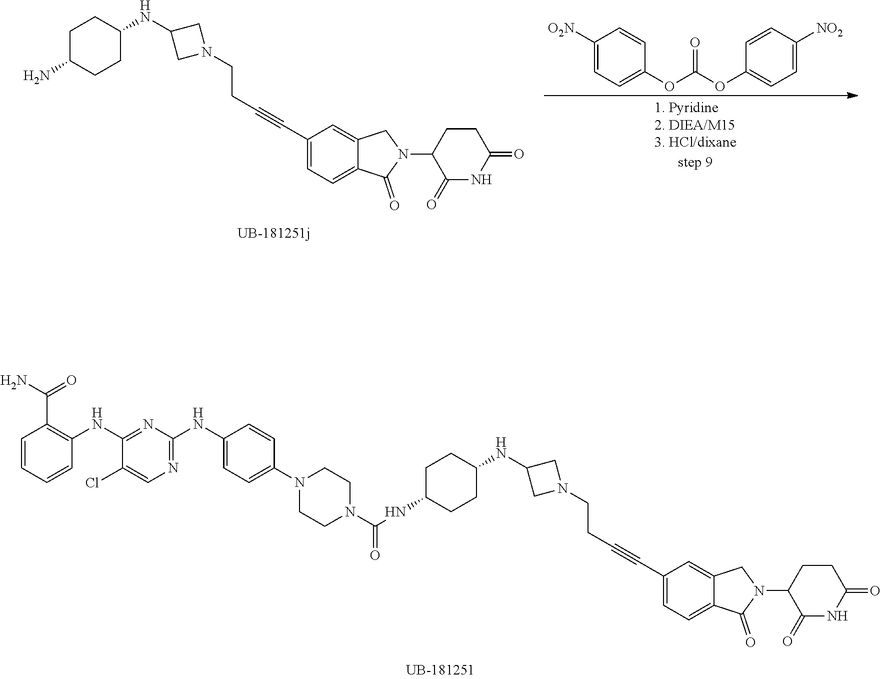 [SMILES: N[C@H]1CC[C@@H](NC2CN(CCC#Cc3ccc4c(c3)CN(C3CCC(=O)NC3=O)C4=O)C2)CC1 ; O=C(Oc1ccc([N+](=O)[O-])cc1)Oc1ccc([N+](=O)[O-])cc1 ; "UB-181251j" ; NC(=O)c1ccccc1Nc1nc(Nc2ccc(N3CCN(C(=O)N[C@H]4CC[C@@H](NC5CN(CCC#Cc6ccc7c(c6)CN(C6CCC(=O)NC6=O)C7=O)C5)CC4)CC3)cc2)ncc1Cl]

### Step 1: UB-181251b

Compound UB-181251a (800 mg, 6.96 mmol) and 1-benzoylazetidin-3-one (1255 mg, 5.30 mmol) were dissolved in dichloromethane (25 mL). After reacting for 3 hours, sodium cyanoborohydride (1700 mg, 17.9 mmol) was added and the mixture was reacted for 20 h at room temperature. Water (10 mL) was added and the organic solvent was removed by rotary evaporator under reduced pressure, then the residue was treated with dichloromethane and the organic layer was washed with saturated NaHCO3 solution. After drying over anhydrous Na2SO4, the solvent was removed under reduced pressure and the residue was purified by rapid column chromatography using CHCl3/MeOH (volume ratio of 9:1) as eluent. Compound UB-181251b (890 mg, 50% yield) was obtained as white solid. LCMS [M+H]⁺=337.5

### Step 2: UB-181251c

UB-181251b (200 mg, 0.60 mmol) and (Boc)₂O (160 mg, 0.74 mmol) were mixed and dissolved in tBuOH (10 mL), and then added with tBuOK (82 mg). The mixture was reacted for 30 min at room temperature under N₂ protection. Then the reaction was heated to 60° C. for 8 h, and then cooled to room temperature. The reaction was treated with dichloromethane and the organic layer was washed with saturated NaHCO3 solution. After drying over Na2SO4, the solvent was removed under reduced pressure and the residue was purified by rapid column chromatography using cyclohexane/ethyl acetate (7:3) as eluent. Compound UB-181251c (195 mg, 75% yield) was obtained. LCMS [M+H]⁺=437.5

### Step 3: UB-181251d

UB-181251c (50 mg, 0.10 mmol), and 10% palladium carbon (5 mg) were added to a mixed solvent of methanol/dichloromethane (1 mL/10 mL), and reacted at room temperature for 16 hours under hydrogen atmosphere. After filtration, the filtrate was concentrated to obtain crude product. The crude product was washed with cold ether (10 mL*3), and dried to obtain compound UB-181251d (39 mg, yield 83%). LCMS [M+H]⁺=271.2

### Step 4: UB-181251e

Compound UB-181251d (0.72 g, 2.14 mmol), ethyl trifluoroacetate (0.37 g, 2.60 mmol) and DIEA (1.38 g, 10 mmol) were added to anhydrous DCM (15 mL). The mixture was reacted at 80° C. for 18 hours. Upon completion of the reaction, the reaction solution was concentrated. The crude product was isolated by rapid column chromatography (DCM/MeOH=10/1) to obtain compound UB-181251e (464 mg, yield 75%) as colorless oil. LCMS [M+H]+=367.6

### Step 5: UB-181251f

Compound UB-181251e (500 mg, 0.984 mmol), methanesulfonyl chloride (145 mg, 1.28 mmol) and triethylamine (149 mg, 1.476 mmol) were successively added to dichloromethane (10 mL), and the mixture was reacted at 25° C. overnight. Upon completion of the reaction, the reaction was poured into 10 mL of water and extracted with dichloromethane (10 mL*3). The organic phases were combined, then washed with saturated saline, dried over anhydrous Na₂SO₄, concentrated by rotary evaporation under reduced pressure to obtain crude product. The crude product was purified by rapid column chromatography (DCM/MeOH=10/1) to obtain compound UB-181251f as white solid (560 mg, yield 97%). LCMS [M+H]⁺=445.7

### Step 6: UB-181251g

UB-181251f (3 g, 5 mmol) was dissolved in DMF (100 mL), and added with sodium azide (0.43 g, 7 mmol). The mixture was stirred at 85° C. overnight. Upon completion of the reaction, the mixture was filtered to obtain filtrate and the filtrate was concentrated in vacuum to obtain the crude product, which was purified by column chromatography separation (DCM/MeOH=30/1) to obtain UB-181251g (2.7 g, 98% yield) as colourless oil. LCMS [M+H]⁺=533.6

### Step 7: UB-18181251h

Compound UB-181251g (6 g, 18 mmol), and sodium hydroxide (1.42 g, 36 mmol) were added to methanol (50 mL) successively, and reacted at 30° C. for 16 hours. Upon completion of the reaction, the reaction was concentrated and the aqueous phase was acidified with hydrochloric acid (1M) to pH=5. Then the mixture was extracted with dichloromethane (10 ml*3), and the combined organic layer was dried over anhydrous Na₂SO₄, and concentrated to obtain white compound UB-181251h (3.5 g, yield 81%). LCMS [M+H]⁺=339.4

### Step 8: UB-181251i

Compound UB-181251h (78 mg, 0.20 mmol), UB-181251h-1 (51 mg, 0.20 mmol) and DIEA (50 mg, 0.39 mmol) were added to anhydrous acetonitrile (30 mL). The mixture was reacted at 80° C. for 18 hours. Upon completion of the reaction, the reaction solution was concentrated. The crude product was isolated by silica gel column chromatography (DCM/MeOH=10/1) to obtain compound UB-181251i (51 mg, yield 46%).

### Step 9: UB-181251j

UB-181251i (50 mg, 0.081 mmol) was dissolved in THF (10 mL), and added with trimethylphosphine (402 mg, 1.87 mmol). The mixture was reacted at room temperature overnight. Upon completion of the reaction, the reaction solution was concentrated to obtain crude product, which was purified by rapid chromatography (DCM/MeOH=10/1) to obtain compound UB-181251j (45 mg, yield 96%).

### Step 10: UB-181251

Synthesised using a method similar to General Method 6. LCMS [M+H]⁺=915.1

### Synthesis of Compound UB-181257

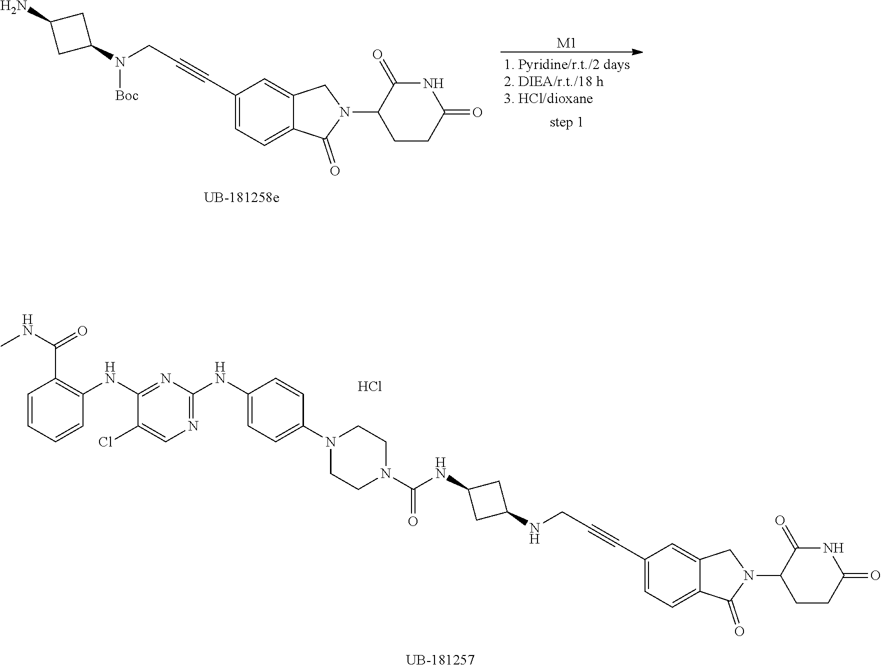 [SMILES: CC(C)(C)OC(=O)N(CC#Cc1ccc2c(c1)CN(C1CCC(=O)NC1=O)C2=O)[C@H]1C[C@@H](N)C1 ; "M1" ; CNC(=O)c1ccccc1Nc1nc(Nc2ccc(N3CCN(C(=O)N[C@H]4C[C@@H](NCC#Cc5ccc6c(c5)CN(C5CCC(=O)NC5=O)C6=O)C4)CC3)cc2)ncc1Cl ; Cl]

### Step 1: UB-181257

Synthesised using a method similar to General Method 6. ¹H NMR (400 MHz, DMSO-d₆) δ 11.76 (s, 1H), 11.01 (s, 1H), 9.66 (s, 2H), 9.49 (s, 1H), 8.82-8.68 (m, 2H), 8.21 (s, 1H), 7.83-7.73 (m, 3H), 7.64 (d, J=7.9 Hz, 1H), 7.58-7.44 (m, 3H), 7.20-7.04 (m, 3H), 6.99 (m, 1H), 5.13 (dd, J=13.3, 5.1 Hz, 1H), 4.50 (d, J=17.7 Hz, 1H), 4.37 (d, J=17.7 Hz, 1H), 4.17 (t, J=4.7 Hz, 2H), 3.93 (dm, 1H), 3.54 (m, 4H), 3.15 (m, 2H), 2.94-2.88 (m, 1H), 2.81 (d, J=4.4 Hz, 3H), 2.59 (m, 4H), 2.40 (m, 2H), 2.23 (m, 2H), 2.06-1.96 (m, 2H). LCMS [M/2+H]⁺=416.

### Synthesis of Compound UB-181258

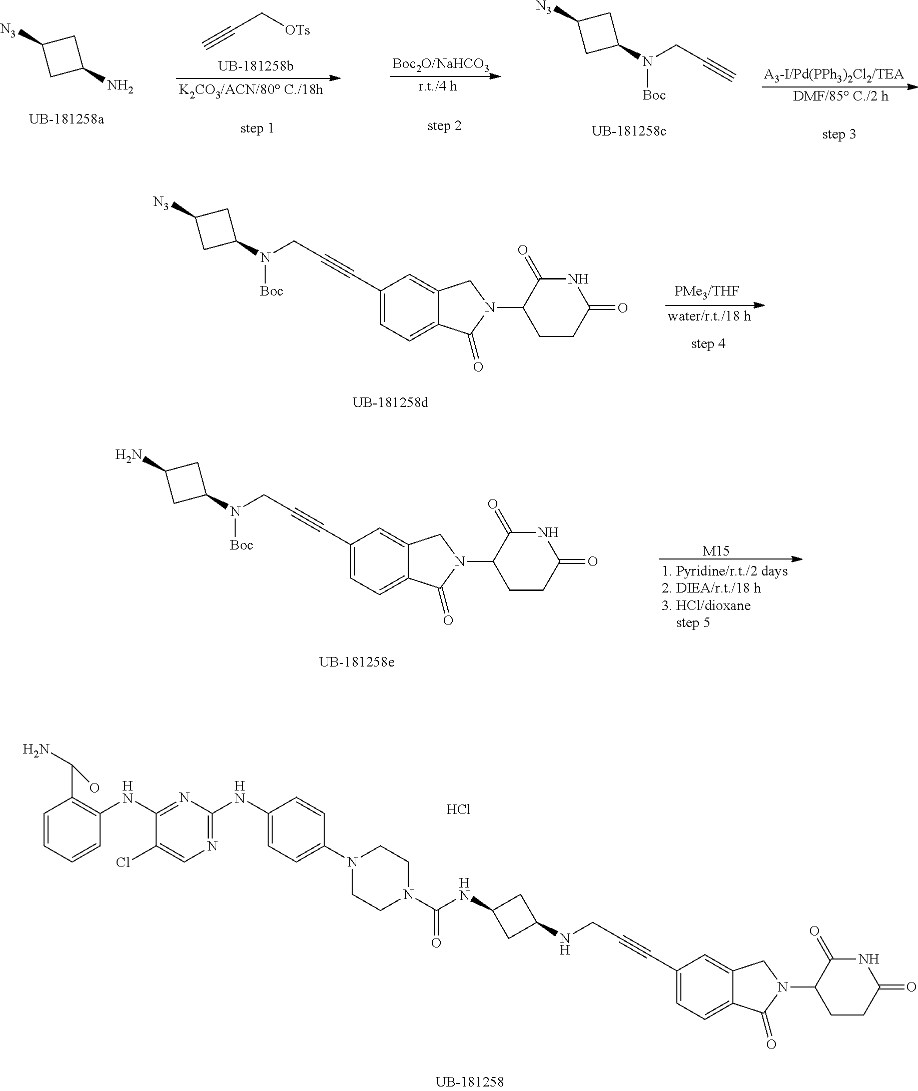

### Step 1 & 2: UB-181258c

Compound UB-181258a (0.4 g, 2.7 mmol) was dissolved in ACN (50 mL), and added with UB-181258b (0.56 g, 2.7 mmol), K₂CO₃ (0.447 g, 3.24 mmol) and reacted at 60° C. overnight. The reaction solution was filtered after cooling. The filtrate was added with aq. NaHCO₃ (3 mL) and Boc₂O (1 mL) and reacted for 4 hours at room temperature. The reaction solution was concentrated, and the crude product was separated by column chromatography (PE/EtOAc=0-10%) to afford product UB-181258c (300 mg, 44% yield) as colorless oil. LCMS [M+H]⁺=251.3. ¹H NMR (400 MHz, chloroform-d) δ 4.05-3.98 (m, 2H), 3.60-3.52 (m, 1H), 3.46 (brs, 1H), 2.70-2.61 (m, 2H), 2.32-2.24 (m, 2H), 2.19 (t, J=2.4 Hz, 1H), 1.47 (s, 9H).

### Step 3: UB-181258d

A mixture of UB-181258c (300 mg, 1.2 mmol), A3-I (444 mg, 1.2 mmol), Pd(PPh₃)₂Cl₂ (84 mg, 0.12 mmol), CuI (23 mg, 0.12 mmol), and TEA (121 mg, 0.12 mmol) was dissolved in dry DMF (5 mL) and reacted at 80° C. for 2 h under N2 protection. The reaction solution was concentrated, and the crude product was separated by column chromatography (DCM/MeOH=0-3%) to give product UB-181258d (200 mg, 34% yield) as yellow oil. LCMS [M+H]⁺=493.6

### Step 4: UB-181258e

Compound UB-181258d (100 mg, 0.2 mmol) was dissolved in THF (5 mL), and added with 1M Me₃P (1 mL, 1 mmol), and the mixture was reacted at room temperature overnight. The reaction solution was concentrated to obtain crude UB-181258e (70 mg, yield 75%) as yellow solid. LCMS [M+H]⁺=467.6

### Step 5: UB-181258

Synthesised using a method similar to General Method 6. ¹H NMR (400 MHz, DMSO-d₆) δ 12.07 (s, 1H), 11.01 (s, 1H), 9.77 (s, 2H), 9.62 (s, 1H), 8.74 (s, 1H), 8.34 (s, 1H), 8.23 (s, 1H), 7.88-7.75 (m, 4H), 7.66-7.46 (m, 4H), 7.15 (m, 3H), 7.05 (m, 1H), 5.13 (dd, J=13.3, 5.1 Hz, 1H), 4.50 (d, J=17.7 Hz, 1H), 4.37 (d, J=17.6 Hz, 1H), 4.14 (m, 2H), 3.94 (m, 1H), 3.59 (m, 4H), 3.21 (m, 2H), 2.92 (m, 2H), 2.60 (m, 4H), 2.40 (m, 1H), 2.25 (m, 2H), 2.02 (m, 2H). LCMS [M/2+H]⁺=409.

### Synthesis of Compound UB-181259

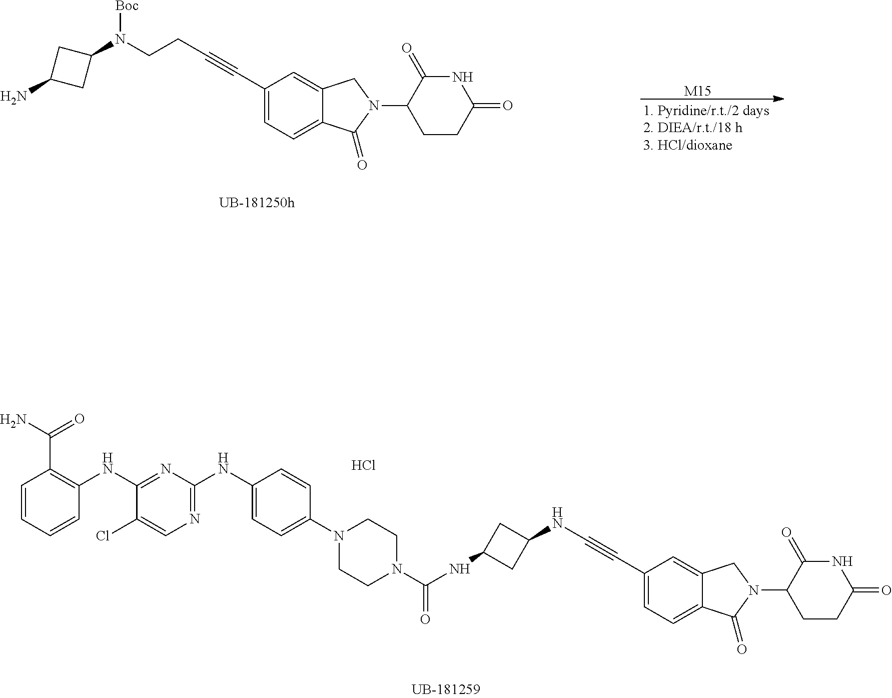 [SMILES: CC(C)(C)OC(=O)N(CCC#Cc1ccc2c(c1)CN(C1CCC(=O)NC1=O)C2=O)[C@H]1C[C@@H](N)C1 ; "M15" ; NC(=O)c1ccccc1Nc1nc(Nc2ccc(N3CCN(C(=O)N[C@H]4C[C@@H](NC#Cc5ccc6c(c5)CN(C5CCC(=O)NC5=O)C6=O)C4)CC3)cc2)ncc1Cl ; Cl]

### Step 1: UB-181259

Synthesised using a method similar to General Method 6. ¹H NMR (400 MHz, DMSO-d₆) δ 12.22 (s, 1H), 11.00 (s, 1H), 9.94 (s, 1H), 9.54 (s, 2H), 8.68 (s, 1H), 8.38 (s, 1H), 8.29 (s, 1H), 7.92-7.80 (m, 2H), 7.76-7.69 (m, 2H), 7.61 (m, 3H), 7.52 (t, J=7.9 Hz, 1H), 7.43 (s, 2H), 7.19 (t, J=7.5 Hz, 2H), 5.11 (dd, J=13.3, 5.1 Hz, 1H), 4.47 (d, J=17.6 Hz, 1H), 4.34 (d, J=17.5 Hz, 1H), 3.93 (m, 1H), 3.35 (m, 4H), 3.09 (t, J=5.9 Hz, 2H), 2.97-2.91 (m, 2H), 2.60 (d, J=11.9 Hz, 3H), 2.45-2.36 (m, 2H), 2.28 (m, 2H), 2.09-1.92 (m, 2H). LCMS [M/2+H]⁺=416.1.

### Synthesis of Compound UB-181261

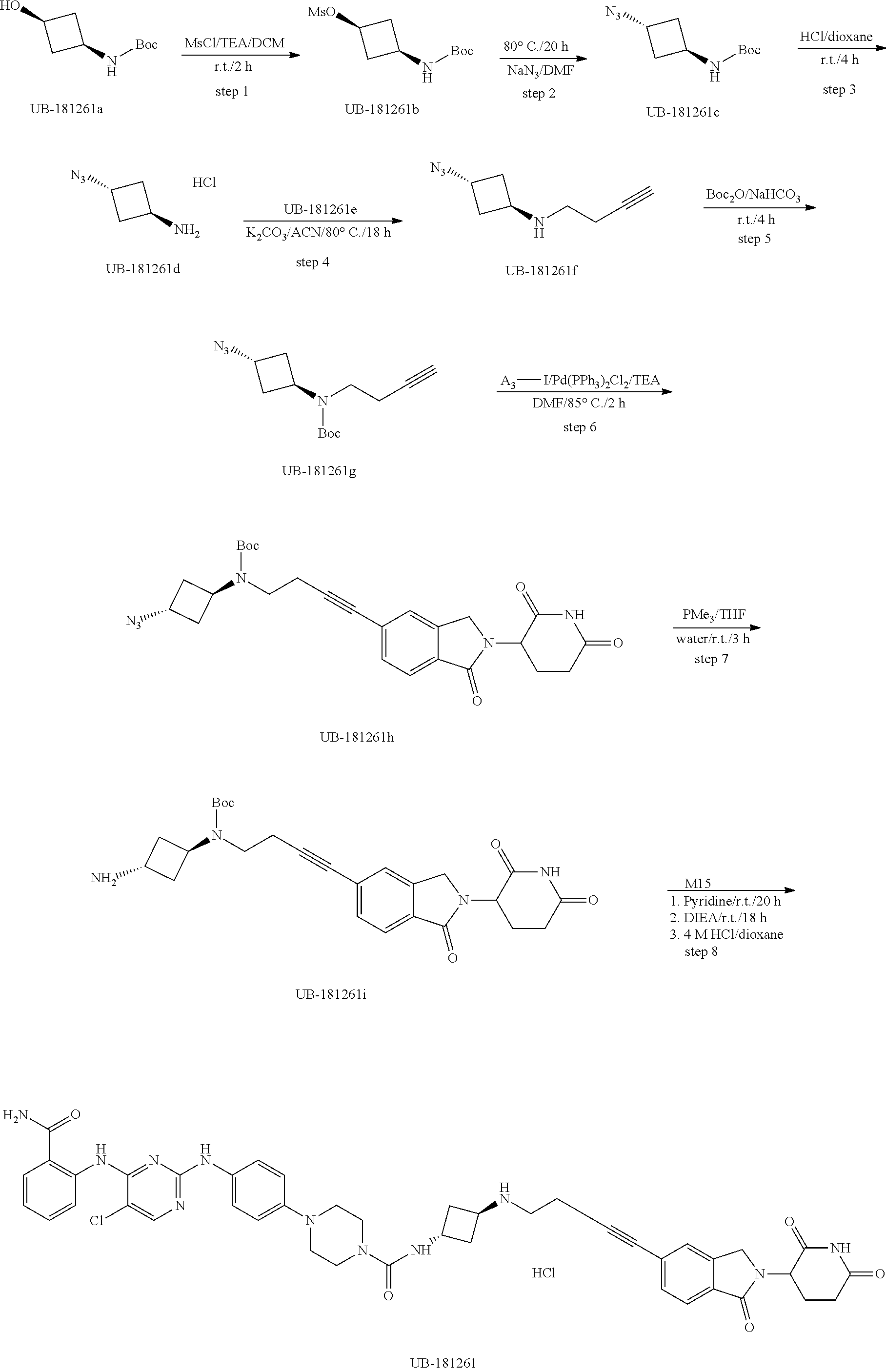

### Step 1: UB-181261b

A solution of Compound UB-181261a (2 g, 10.68 mmol) in DCM (20 mL) was added with TEA (2.1 g, 21.36 mmol) and MsCl (1.8 g, 16.02 mmol), and then stirred at room temperature for 2 hours. The reaction solution was added with water and extracted with DCM (30 mL*2), the organic layer was dried over Na₂SO₄, and the residue was purified by silica gel chromatography (PE/EtOAc=0-40%) to afford product UB-181261b (2.6 g, 93% yield) as white solid. LCMS [M+H]⁺=266.3.

### Step 2: UB-181261c

Compound UB-181261b (2.6 g, 9.8 mmol) and NaN₃ (1.27 g, 19.6 mmol) were dissolved in DMF (30 mL), and reacted at 85° C. overnight. The reaction solution was added with saturated saline (20 mL), and then extracted with EtOAc (30 mL*2). The organic phase was washed with water and saline, dried, and concentrated to obtain yellow solid product UB-181261c (1.8 g, yield 86.5%). LCMS [M+H]⁺=213.2

### Step 3: UB-181261d

Compound B-181261c (1.8 g, 8.5 mmol) was dissolved in DCM (10 mL), 4M HCl/dioxane (10.6 mL, 42.5 mmol) was added, and the mixture was reacted at room temperature for 4 hours. The solvent was spin-dried to obtain crude product UB-181261d (1.3 g, yield 100%) as white solid. LCMS [M+H]+=113.1

Step 4 & 5: UB-181250g

Compound UB-181261-d (0.5 g, 3.4 mmol) in ACN (30 mL) was added with UB-181261e (0.9 g, 4 mmol), and K2CO3 (0.56 g, 4 mmol) and reacted at 80° C. overnight. The reaction solution was filtered after cooling. The filtrate was added with aq. NaHCO₃ (3 mL) and Boc₂O (1.5 mL) and reacted for 4 hours at room temperature. The reaction solution was concentrated, and the crude product was separated by column chromatography (PE/EtOAc=0-20%) to give product UB-181250g (400 mg, 44% yield) as colorless oil. LCMS [M+H]+=265.3

### Step 6: UB-181261h

A mixture of UB-181250g (60 mg, 0.23 mmol), A3-I (85 mg, 0.38 mmol), Pd(PPh3)₂Cl₂ (16 mg, 0.038 mmol), CuI (4.4 mg, 0.038 mmol) and TEA (23 mg, 0.38 mmol) was dissolved in dry DMF (4 mL), and reacted at 80° C. for 2 hours under N2 protection. The reaction solution was concentrated and the crude product was separated by column chromatography (DCM/MeOH=0-10%) to give product UB-181261h (49 mg, 42% yield) as brown solid. LCMS [M+H]+=507.5

### Step 7: UB-181261i

Compound UB-181261h (49 mg, 0.097 mmol) was dissolved in THF (5 mL), 1M Me3P (0.5 mL, 0.5 mmol) was added, and the mixture was reacted at room temperature for 1 hour. Then water (0.5 mL) was added, and the mixture was reacted overnight. The reaction solution was concentrated, and the crude product was purified by thin layer chromatography (EtOAc) to give product UB-181261i (26 mg, 56% yield) as yellow oil. LCMS [M+H]+=481.5

Step 8: UB-181261 was synthesised using a method similar to General Method 6. LCMS [M/2+H]⁺=433.2.

### Synthesis of Compound UB-181269

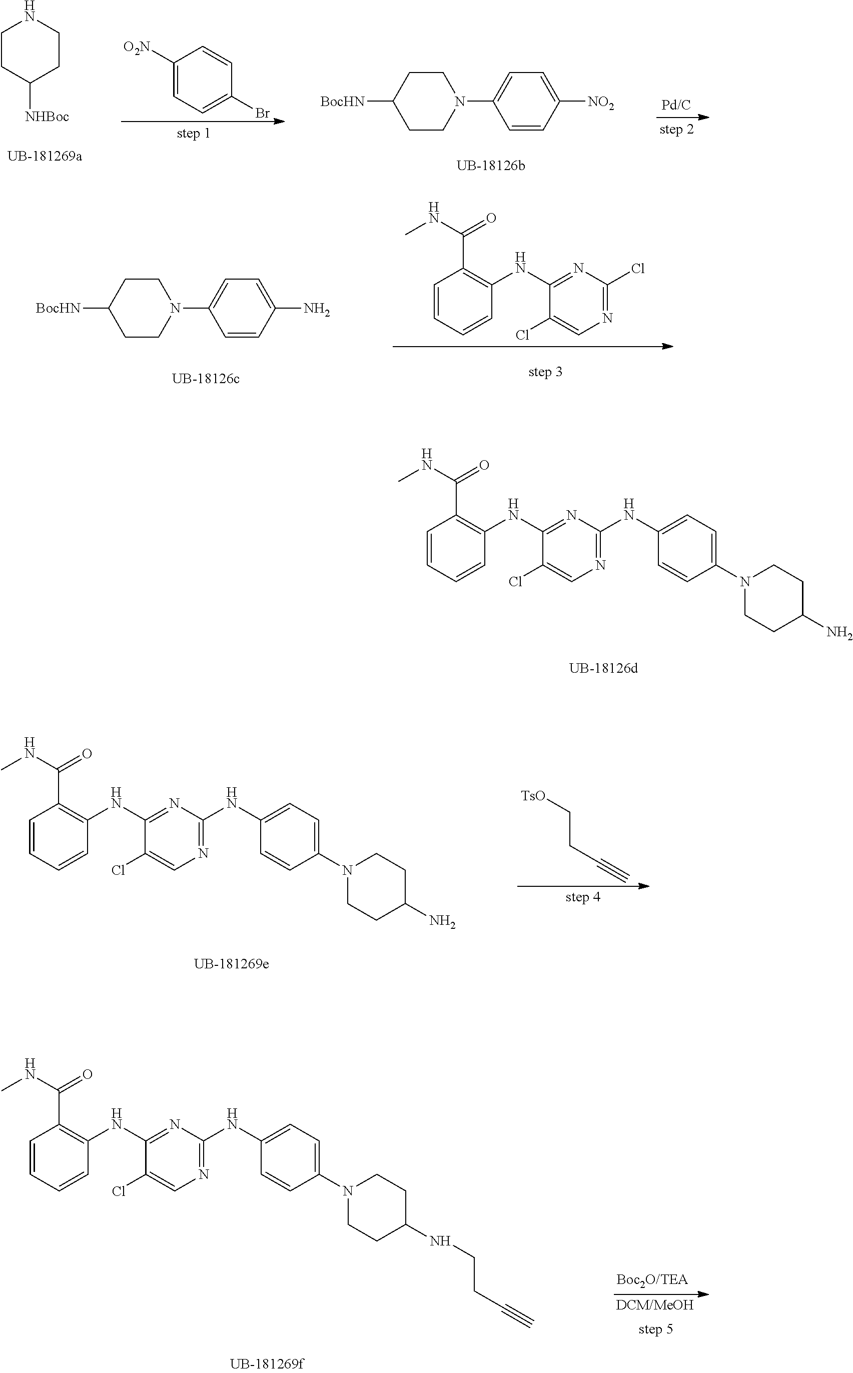

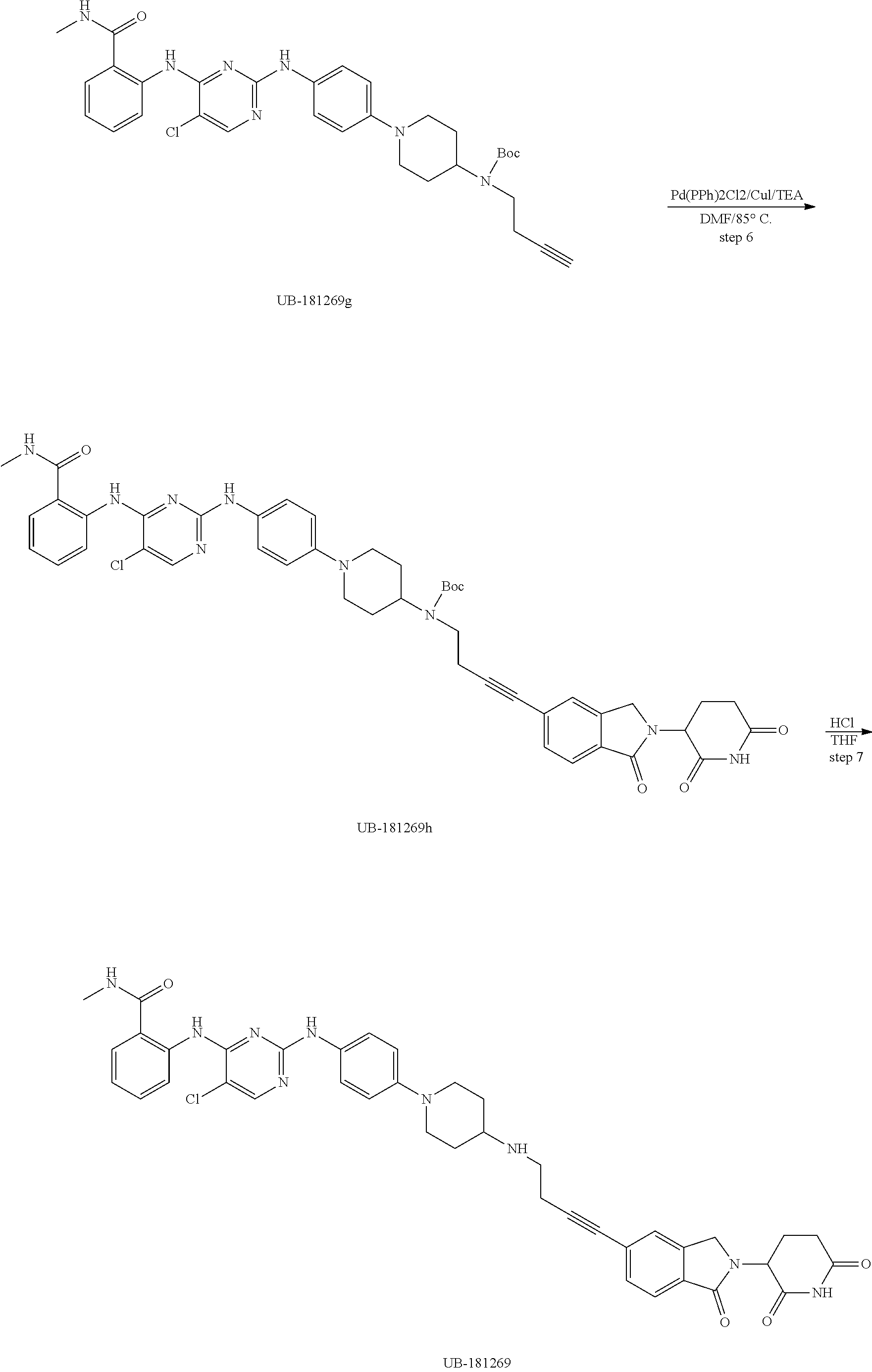

### Step 1: UB-181269b

Compound UB-181269a (1.00 g, 5.0 mmol), 1-bromo-4-nitrobenzene (1.01 g, 5.0 mmol) and DIEA (1.29 g, 10.0 mmol) were added to anhydrous acetonitrile (30 mL). The mixture was reacted at 80° C. for 18 hours. Upon completion of the reaction, the reaction solution was concentrated. The crude was isolated by silica gel column chromatography (DCM/MeOH=10/1) to obtain compound UB-181269b (1.38g, yield 86%). LCMS: [M+H]⁺=322.3

### Step 2: UB-181269c

UB-181269b (1.38 g, 4.3 mmol), and 10% Pd/C (130 mg) were added to methanol (80 mL), and reacted at room temperature for 16 hours under hydrogen atmosphere. After filtration, the filtrate was concentrated to obtain crude product. The crude product was washed with cold ether (10 mL*3), and dried to obtain compound UB-181269c (1.25g, yield 100%). LCMS: [M+H]⁺=292.3.

### Step 3: UB-181269d

UB-181269c (780 mg, 2.68 mmol), con HCl (0.01 mL) and UB-181269a-1 (796 mg, 2.68 mmol) were dissolved in MeCN (90 mL) and stirred for 18 h at 80° C. Then the mixture was purified by silica gel chromatography (petroleum ether/ethyl acetate=70% to 100% for 20 min, then MeOH/DCM=O % to 10% for 40 min) to obtain Compound UB-181269d (822 mg, yield 68%). LCMS: [M+H]⁺=452.9

### Step 4: UB-181269e

UB-181269d (300 mg, 0.67 mmol), DIEA (100 mg, 0.78 mmol) and 3-butyl p-toluenesulfonate (246 mg, 1.1 mmol) were dissolved in acetonitrile (30 mL) and stirred for 18 hours at 80° C. Then the mixture was purified by rapid column chromatography (petroleum ether/ethyl acetate=70% to 100% for 20 min, then MeOH/DCM=0% to 10% for 40 min) to obtain Compound UB-181269e (187 mg, yield 56%). LCMS: [M+H]⁺=505.3

### Step 5: UB-181269f

Compound UB-181269e (187 mg, 0.37 mmol), di-tert-butyl dicarbonate (160 mg, 0.74 mmol) and triethylamine (82 mg) were added to tetrahydrofuran (20 mL) successively. The mixture was reacted at room temperature for 2 hours. Upon completion of the reaction, the reaction was poured into 10 mL of water and extracted with dichloromethane (5 mL*3). The organic phases were combined, then washed with saturated saline, dried over anhydrous Na₂SO₄, and concentrated by rotary evaporation under reduced pressure to obtain compound UB-181269f (165 mg, yield 74%) as colorless oil. LCMS [M+H]⁺=605.5

### Step 6: UB-181269g

Compound UB-181269f (30 mg, 0.050 mmol) and A3-I (38 mg, 0.103 mmol) were dissolved in DMF (10 mL), and added with dichlorobis(triphenylphosphonium)palladium (7.2 mg, 0.010 mmol), cuprous iodide (3.91 mg, 0.021 mmol) and triethylamine (150 mg, 1.49 mmol). The mixture was reacted at 80° C. overnight under nitrogen. The reaction solution was filtered through diatomite and the filtrate was concentrated to obtain crude product, which was purified by rapid chromatography (elution with DCM/MeOH=0%-20% for 30 min) to give product UB-181269g (10 mg, 24% yield). LCMS [M+H]⁺=847.4

### Step 7: UB-181269

Compound UB-181269g (10 mg, 0.012 mmol) and hydrochloric acid in dioxane (10 mL, 4 N) were added to tetrahydrofuran (10 mL) and reacted at room temperature for 2 h. Upon completion of the reaction, the reaction was concentrated by rotary evaporation under reduced pressure to give compound UB-181269 (5.6 mg, 100% yield). LCMS [M+H]⁺=747.1. ¹H NMR (400 MHz, DMSO-d₆) δ 11.80 (s, 1H), 11.00 (s, 1H), 9.78-9.69 (m, 1H), 9.68-9.47 (m, 2H), 8.80 (s, 1H), 8.46 (s, 1H), 8.16 (s, 1H), 7.87-7.66 (m, 5H), 7.75 (dt, J=15.4, 9.6 Hz, 1H), 7.63-7.49 (m, 2H), 7.18 (t, J=7.5 Hz, 1H), 5.12 (dd, J=13.2, 5.0 Hz, 1H), 4.56-4.35 (m, 2H), 3.44-3.11 (m, 4H), 3.00 (s, 3H), 2.89-2.70 (m, 7H), 2.68 (s, 3H), 2.61 (d, J=9.9 Hz, 2H), 2.35 (dd, J=3.3, 7.5 Hz, 4H), 2.19-2.08 (m, 2H), 2.01-1.93 (m, 2H).

### Synthesis of Compound UB-181270

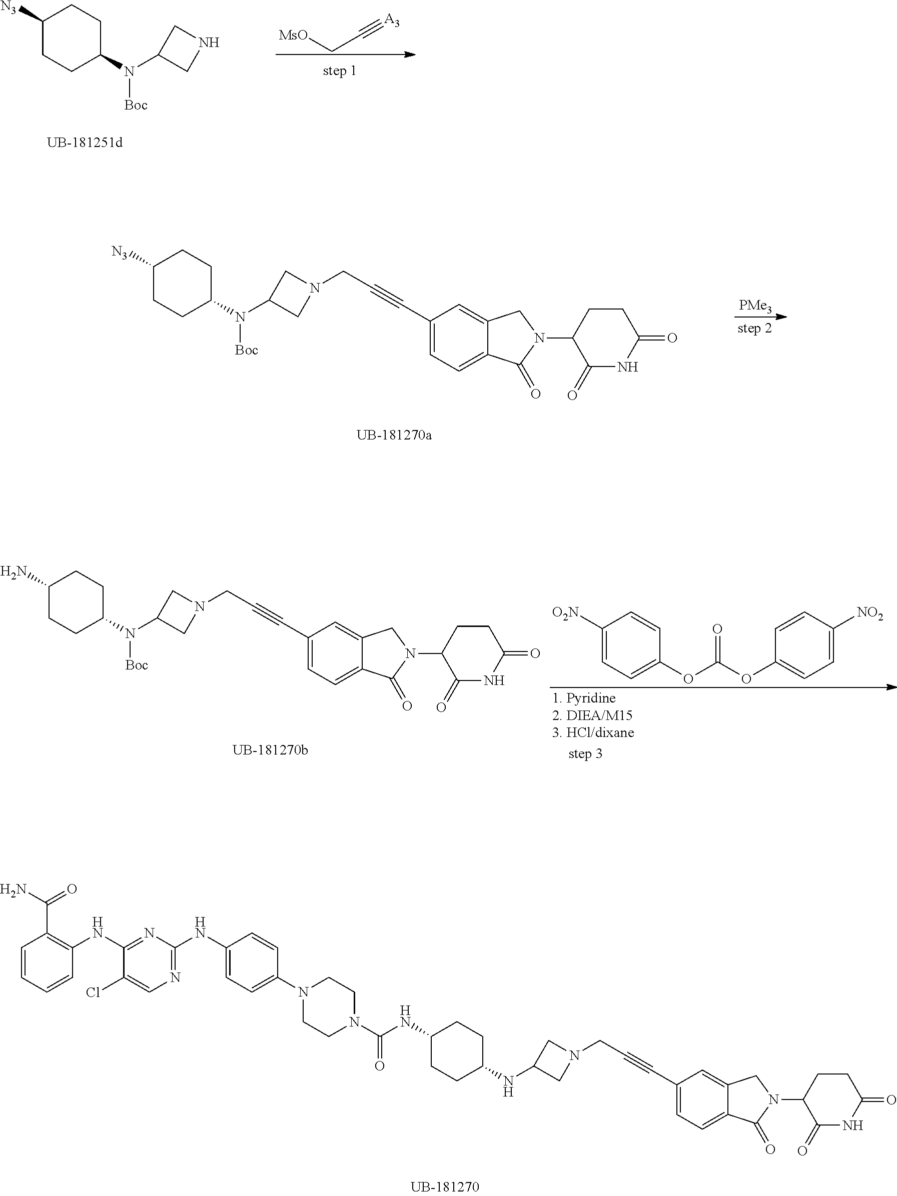

### Step 1: UB-181270a

Compound UB-181251d (180 mg, 0.61 mmol), UB-181251d-1 (468 mg, 1.20 mmol) and DIEA (100 mg, 0.78 mmol) were successively added to anhydrous acetonitrile (30 mL). The mixture was reacted at 80° C. for 18 hours. Upon completion of the reaction, the reaction solution was concentrated. The crude product was isolated by column chromatography (DCM/MeOH=10/1) to obtain compound UB-181270a (161 mg, yield 46%). LCMS [M+H]⁺=576.6

### Step 2: UB-181270b

UB-181270a (161 mg, 0.28 mmol) was dissolved in THF (10 mL), and added with trimethylphosphine (402 mg, 1.87 mmol). The mixture was reacted at room temperature overnight. Upon completion of the reaction, the reaction solution was concentrated to obtain crude product, which was purified by rapid chromatography (DCM/MeOH=10/1) to obtain compound UB-181270b (147 mg, yield 96%). LCMS [M+H]⁺=550.6

### Step 3: UB-181270

The method was similar to General Method 6. LCMS [M+H]⁺=900.2

### Synthesis of Compound UB-181272

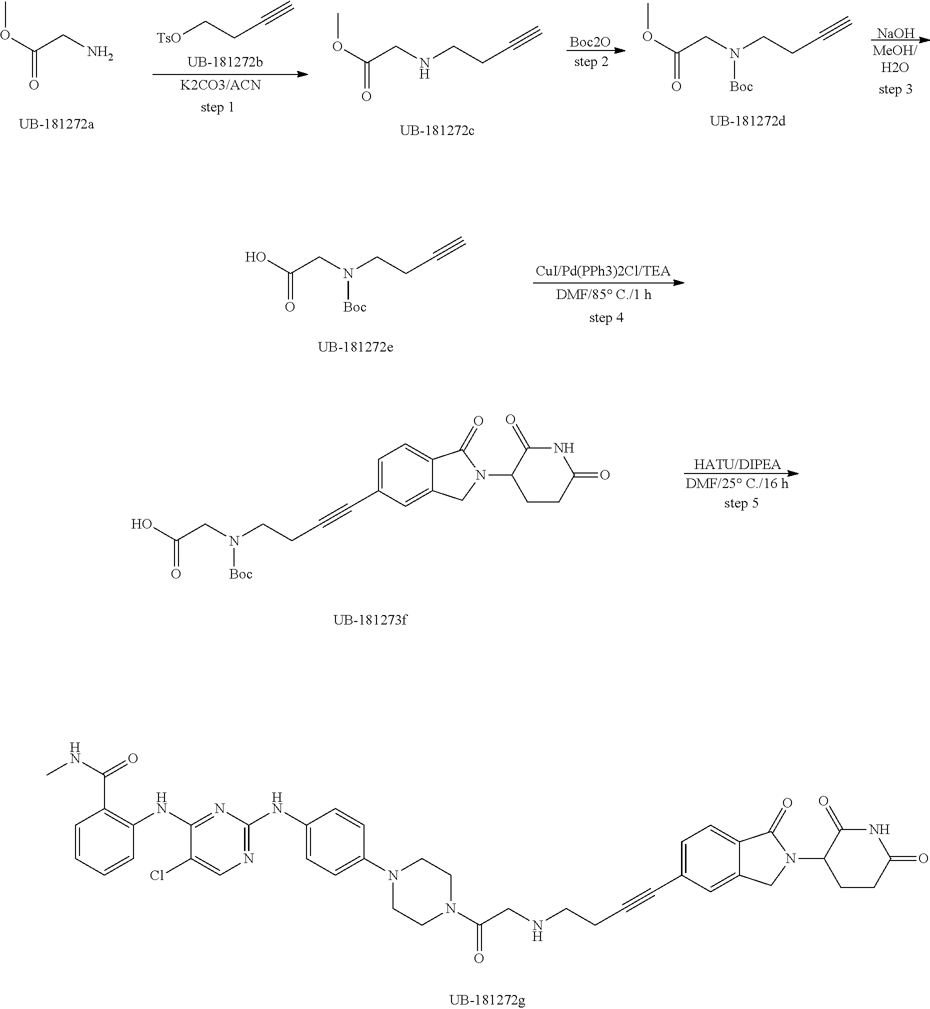

### Step 1 and 2: UB-181272d

Compound UB-181272a (1000 mg, 11.24 mmol) was dissolved in ACN (40 mL), and added with UB-181272b (1.76 g, 7.87 mmol), and K₂CO₃ (2.17 g, 15.7 mmol) and then reacted at 80° C. overnight. The reaction solution was filtered after cooling. The filtrate was added with aq. NaHCO₃ (3 mL) and Boc₂O (2.5 mL) and then reacted for 4 hours at room temperature. The reaction solution was concentrated, and the crude product was separated by column chromatography (PE/EtOAc=0-10%) to give product UB-181272d (850 mg, 53.6% yield) as colorless oil. LCMS [M+H]⁺=242.2

### Step 3: UB-181272e

Compound UB-181071d (200 mg, 0.83 mmol) was dissolved in ethanol (5 mL), and added with 2M NaOH (2 mL). The mixture was reacted for 18 hours at room temperature. The reaction solution was concentrated and added with water (3 mL), then extracted with ether (10 mL*3). The organic impurities were removed. The aqueous phase was neutralised to pH˜6 with 1 M HCl and lyophilizated to give product UB-181272e (120 mg, 35.1% yield) as white solid. LCMS [M+H]⁺=227.3

### Step 4: UB-181272f

UB-181272e (30 mg, 0.1 mmol), A3-I (73 mg, 0.2 mmol), Pd(PPh₃)₂Cl₂ (4.64 mg), CuI (3 mg), and TEA (40 mg) were added to anhydrous DMF (2 mL). The reaction system was stirred at 80° C. for 2 hours, and then cooled to room temperature after completion of the reaction. The mixture was added into water, extracted with dichloromethane, washed with saline (30 mL), dried over sodium sulfate, and filtered. After concentration, the residue was isolated by silica gel column chromatography (dichloromethane/methanol=10%) to obtain UB-181272f as yellow solid (50 mg, yield 80.7%). LCMS [M+H]⁺=470.4

### Step 5: UB-181272

The method was similar to General Method 1. LCMS [M+H]⁺=789.9. ¹H NMR (400 MHz, DMSO-d₆) δ 12.04 (s, 1H), 11.02 (s, 1H), 9.97 (s, 1H), 9.34 (s, 2H), 8.91 (d, J=4.8 Hz, 1H), 8.64 (s, 1H), 8.30 (s, 1H), 7.82 (dd, J=8.0, 1.6 Hz, 1H), 7.75-7.69 (m, 2H), 7.62-7.46 (m, 4H), 7.31-7.08 (m, 3H), 5.12 (dd, J=13.3, 5.1 Hz, 1H), 4.49-4.34 (m, 2H), 4.24 (d, J=5.5 Hz, 2H), 3.80 (s, 2H), 3.68 (s, 2H), 3.37 (d, J=18.0 Hz, 2H), 3.30-3.18 (m, 4H), 3.00 (t, J=7.4 Hz, 2H), 2.93-2.87 (m, 1H), 2.81 (d, J=4.4 Hz, 3H), 2.66-2.56 (m, 1H), 2.42-2.33 (m, 1H), 2.05-1.96 (m, 1H).

### Synthesis of Compound UB-181273

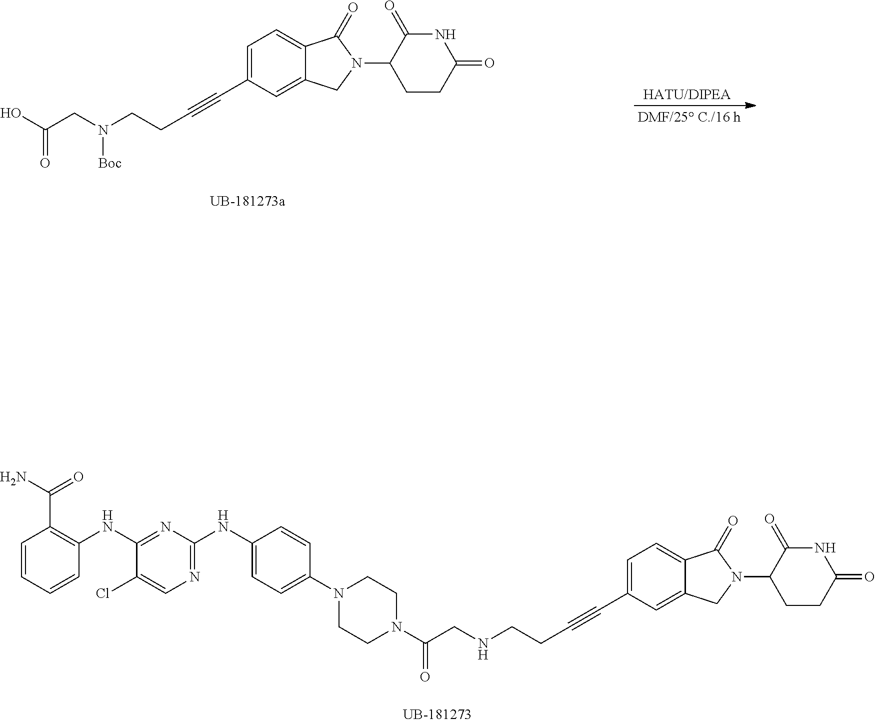 [SMILES: CC(C)(C)OC(=O)N(CCC#Cc1ccc2c(c1)CN(C1CCC(=O)NC1=O)C2=O)CC(=O)O ; NC(=O)c1ccccc1Nc1nc(Nc2ccc(N3CCN(C(=O)CNCCC#Cc4ccc5c(c4)CN(C4CCC(=O)NC4=O)C5=O)CC3)cc2)ncc1Cl]

### Step 1: UB-181273

Synthesised using a method similar to General Method 6. LCMS [M+H]⁺=775.9. ¹H NMR (400 MHz, DMSO-d6) δ 12.23 (s, 1H), 11.02 (s, 1H), 9.86 (s, 1H), 9.31 (s, 2H), 8.70 (s, 1H), 8.40 (s, 1H), 8.28 (s, 1H), 7.90-7.79 (m, 2H), 7.77-7.69 (m, 2H), 7.63-7.45 (m, 4H), 7.17 (t, J=7.6 Hz, 3H), 5.12 (dd, J=13.3, 5.1 Hz, 1H), 4.52-4.33 (m, 2H), 4.23 (s, 2H), 3.77 (s, 2H), 3.65 (s, 2H), 3.31 (s, 2H), 3.23 (d, J=7.9 Hz, 4H), 3.00 (t, J=7.4 Hz, 2H), 2.94-2.86 (m, 1H), 2.70-2.54 (m, 1H), 2.40-2.33 (m, 1H), 2.04-1.94 (m, 1H).

### Synthesis of Compound UB-181274

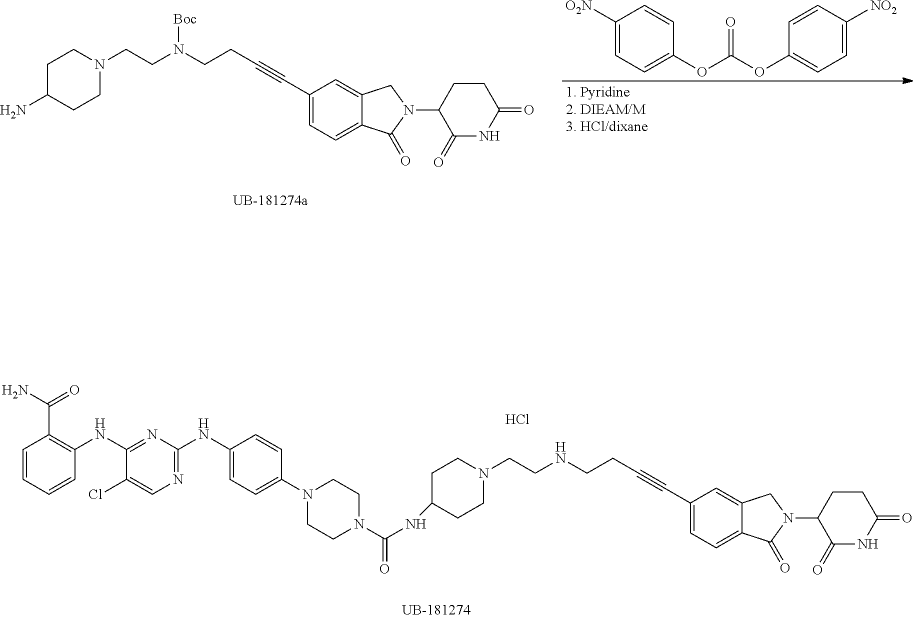 [SMILES: CC(C)(C)OC(=O)N(CCC#Cc1ccc2c(c1)CN(C1CCC(=O)NC1=O)C2=O)CCN1CCC(N)CC1 ; O=C(Oc1ccc([N+](=O)[O-])cc1)Oc1ccc([N+](=O)[O-])cc1 ; NC(=O)c1ccccc1Nc1nc(Nc2ccc(N3CCN(C(=O)NC4CCN(CCNCCC#Cc5ccc6c(c5)CN(C5CCC(=O)NC5=O)C6=O)CC4)CC3)cc2)ncc1Cl ; Cl]

### Step 1: UB-181274

Synthesised using a method similar to General Method 6. LCMS [M/2+H]⁺=887.6. ¹H NMR (400 MHz, DMSO-d6) δ 12.23 (s, 1H), 11.02 (s, 1H), 9.85 (s, 1H), 9.30 (s, 2H), 8.71 (s, 1H), 8.39 (s, 1H), 8.28 (s, 1H), 7.88-7.80 (m, 2H), 7.76-7.69 (m, 2H), 7.62-7.45 (m, 5H), 7.22-7.11 (m, 3H), 5.12 (dd, J=13.3, 5.0 Hz, 1H), 4.50-4.30 (m, 2H), 4.23 (d, J=6.0 Hz, 2H), 3.30 (s, 2H), 3.23 (s, 4H), 3.20-3.07 (m, 2H), 3.03-2.81 (m, 4H), 2.60 (d, J=16.5 Hz, 1H), 2.41-2.33 (m, 1H), 2.05-1.97 (m, 1H).

### Synthesis of Compound UBI-1376 (M12)

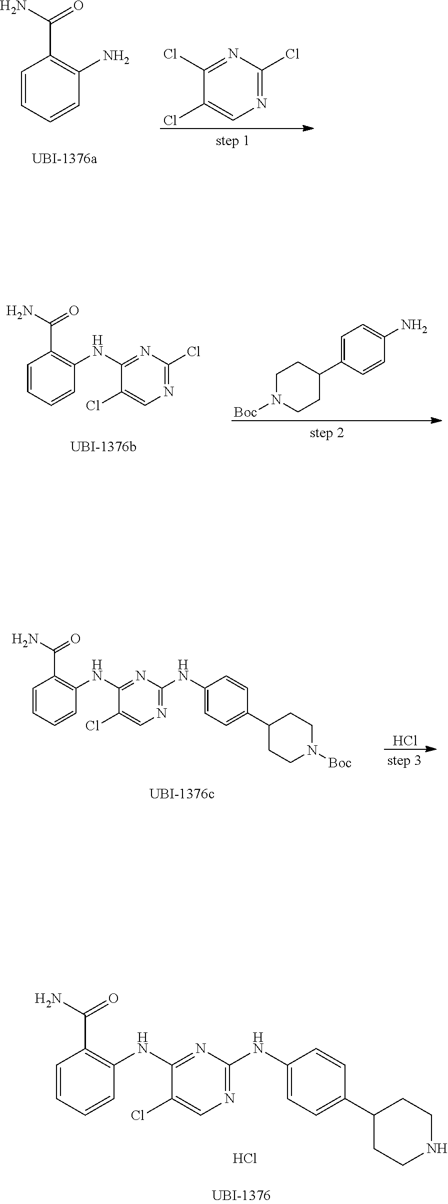

### Step 1: UBI-1376b

Compound 2-aminobenzamide (6.2 g, 45.8 mmol) was placed in a 100 mL three-necked flask, and added with isopropanol (100 mL), 2,4,5-trichloropyrimidine (7 g, 38 mmol), and diisopropylethylamine (8 mL, 45.8 mmol). The mixture was stirred at 80° C. overnight. Upon completion of the reaction, the mixture was cooled to room temperature and then added with 100 ml of water and ethyl acetate. The organic phase was washed with saline, and dried over magnesium sulfate to obtain UB-1376b as yellow solid (9 g, yield 83%). LCMS [M+H]⁺=284.1. ¹H NMR (400 MHz, DMSO) δ 12.50 (s, 1H), 8.60 (d, J=0.6 Hz, 1H), 8.60-8.28 (m, 1H), 8.24 (s, 1H), 7.89 (dd, J=8.0, 1.4 Hz, 2H), 7.72-7.57 (m, 1H), 7.56-7.20 (m, 1H), 7.22 (td, J=7.9, 1.1 Hz, 1H).

### Step 2: UBI-1376c

UBI-1375b (1 g, 4 mmol) and tert-butyl 4-(4-aminophenyl)piperidine-1-carboxylate (1.03 g, 4 mmol) were dissolved in anhydrous DMF (10 mL), and added with Pd(OAc)₂ (120 mg, 1 mmol) and xanphos (310 mg, 1 mmol). The mixture was stirred at 130° C. overnight. Upon completion of the reaction, the reaction was added with water, and extracted with ethyl acetate (10 mL*3). The organic layer was dried over Na2SO4 and concentrated to obtain crude product. The crude product was purified by silica gel chromatography (DCM/MeOH=20/1) to afford the product UBI-1375c (929 mg, 51% yield). LCMS [M+H]⁺=524.1

### Step 3: UBI-1376

Compound UBI-1376c (925 mg, 1.78 mmol) and hydrochloric acid in dioxane (10 mL, 4 N) were added to tetrahydrofuran (10 mL). The mixture was reacted at room temperature for 2 hours. Upon completion of the reaction, the mixture was concentrated by rotary evaporation under reduced pressure to obtain compound UBI-1376 (747 mg, yield 100%). LCMS [M+H]⁺=424.1

### Synthesis of Compound UB-181279

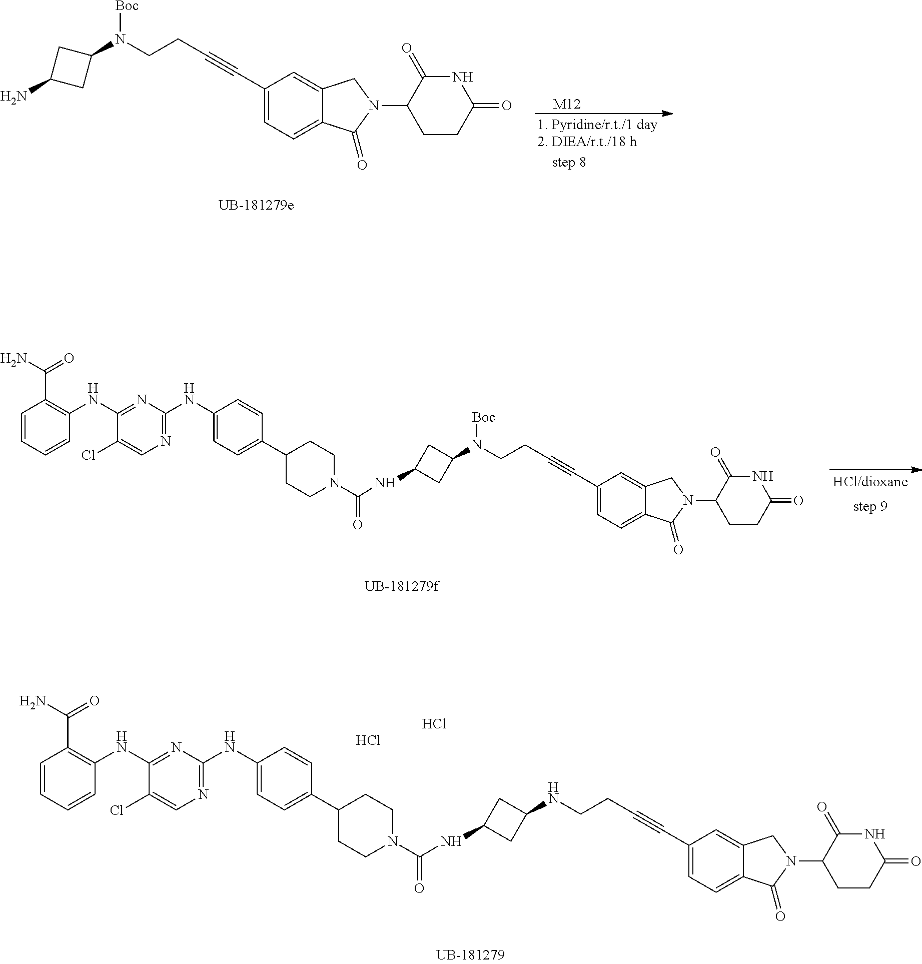 [SMILES: CC(C)(C)OC(=O)N(CCC#Cc1ccc2c(c1)CN(C1CCC(=O)NC1=O)C2=O)[C@H]1C[C@@H](N)C1 ; "M12" ; CC(C)(C)OC(=O)N(CCC#Cc1ccc2c(c1)CN(C1CCC(=O)NC1=O)C2=O)[C@H]1C[C@@H](NC(=O)N2CCC(c3ccc(Nc4ncc(Cl)c(Nc5ccccc5C(N)=O)n4)cc3)CC2)C1 ; NC(=O)c1ccccc1Nc1nc(Nc2ccc(C3CCN(C(=O)N[C@H]4C[C@@H](NCCC#Cc5ccc6c(c5)CN(C5CCC(=O)NC5=O)C6=O)C4)CC3)cc2)ncc1Cl ; Cl ; Cl]

### Step 8: UB-181279f

Compound UB-181279e (95 mg, 0.198 mmol), and bis(4-nitrophenyl)carbonate (120 mg, 0.396 mmol) was dissolved in Py. (1 mL) and the mixture was reacted overnight at room temperature. Compound M12 (90 mg, 0.198 mmol) and DIPEA (51 mg, 0.396 mmol) were added to the above reaction solution and reacted for 2 h at room temperature. The solvent was spin-dried, and the crude product was separated by large plate using preparative TLC (DCM/MeOH=15/1) to obtain product UB-181279f (40 mg, 22% yield) as yellow solid. LCMS [M+H]⁺=930.1

### Step 9: UB-181279

Compound UB-181279f (20 mg, 0.02 mmol) was dissolved in DCM (2 mL), and added with HCl in dioxane (1 mL). The mixture was reacted at room temperature for 1 hour. The reaction solution was added to MTBE (10 ml) and solid appeared. The mixture was stood to clear, and the supernatant was poured out. The above operation was repeated three times. The solid in the bottle was added with water (10 ml) and lyophilizated to give product UB-181279 (8.7 mg, 48% yield) as white solid. LCMS [M/2⁺H]=415.7. ¹H NMR (400 MHz, DMSO) δ 12.04 (s, 1H), 11.00 (s, 1H), 9.62 (s, 1H), 9.40 (s, 2H), 8.74 (d, J=6.8 Hz, 1H), 8.35 (d, J=9.9 Hz, 1H), 8.23 (t, J=3.3 Hz, 1H), 7.84 (d, J=7.9 Hz, 1H), 7.78 (s, 1H), 7.75-7.69 (m, 2H), 7.60 (d, J=8.0 Hz, 1H), 7.55 (d, J=8.3 Hz, 2H), 7.48 (t, J=7.3 Hz, 1H), 7.16 (t, J=8.6 Hz, 3H), 6.86 (s, 1H), 5.11 (dd, J=13.3, 5.1 Hz, 1H), 4.40 (dd, J=51.1, 17.7 Hz, 2H), 4.13 (d, J=12.4 Hz, 2H), 3.89 (d, J=7.9 Hz, 2H), 3.53-3.43 (m, 1H), 3.09 (d, J=5.5 Hz, 2H), 2.97-2.83 (m, 3H), 2.79-2.54 (m, 6H), 2.45-2.31 (m, 2H), 2.24 (d, J=9.8 Hz, 2H), 2.03-1.96 (m, 1H), 1.74 (d, J=12.5 Hz, 2H), 1.53-1.40 (m, 2H).

### Synthesis of Compound UB-181283

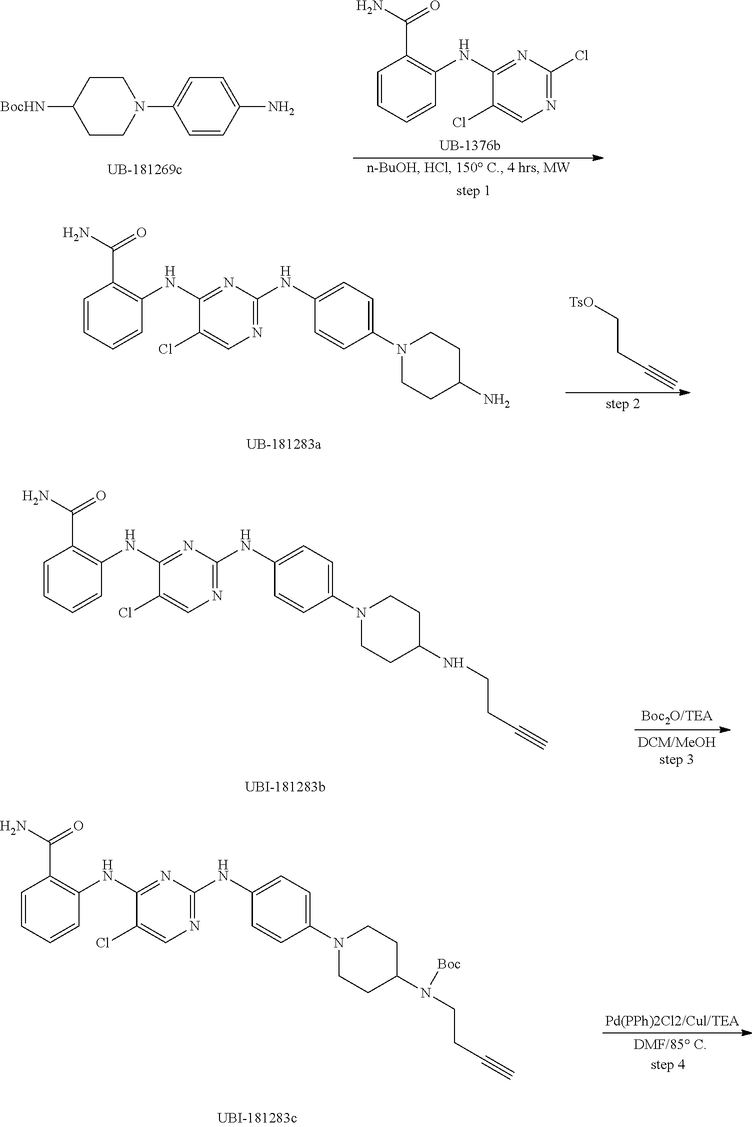 [SMILES: CC(C)(C)OC(=O)NC1CCN(c2ccc(N)cc2)CC1 ; NC(=O)c1ccccc1Nc1nc(Cl)ncc1Cl ; NC(=O)c1ccccc1Nc1nc(Nc2ccc(N3CCC(N)CC3)cc2)ncc1Cl ; C#CCCOS(=O)(=O)c1ccc(C)cc1 ; C#CCCNC1CCN(c2ccc(Nc3ncc(Cl)c(Nc4ccccc4C(N)=O)n3)cc2)CC1 ; C#CCCN(C(=O)OC(C)(C)C)C1CCN(c2ccc(Nc3ncc(Cl)c(Nc4ccccc4C(N)=O)n3)cc2)CC1]

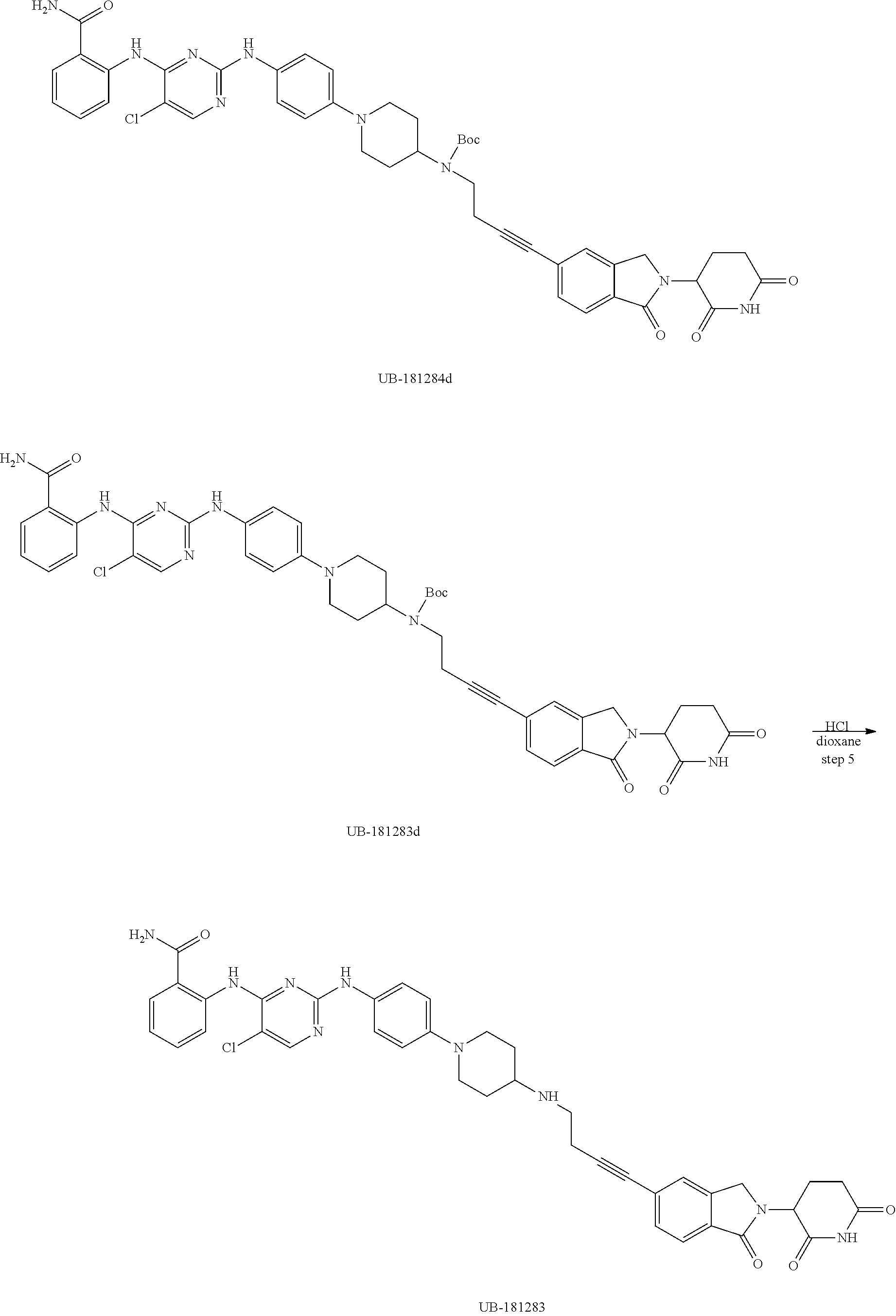 [SMILES: CC(C)(C)OC(=O)N(CCC#Cc1ccc2c(c1)CN(C1CCC(=O)NC1=O)C2=O)C1CCN(c2ccc(Nc3ncc(Cl)c(Nc4ccccc4C(N)=O)n3)cc2)CC1 ; CC(C)(C)OC(=O)N(CCC#Cc1ccc2c(c1)CN(C1CCC(=O)NC1=O)C2=O)C1CCN(c2ccc(Nc3ncc(Cl)c(Nc4ccccc4C(N)=O)n3)cc2)CC1 ; Cl ; NC(=O)c1ccccc1Nc1nc(Nc2ccc(N3CCC(NCCC#Cc4ccc5c(c4)CN(C4CCC(=O)NC4=O)C5=O)CC3)cc2)ncc1Cl]

### Step 1: UB-181283a

UB-181269c (1.0 g, 3.4 mmol), concentrated hydrochloric acid (0.01 mL) and UBI-1376b (970 mg, 3.4 mmol) were dissolved in n-BuOH (90 mL) and the mixture was reacted at 1508° C. for 1 hours. Then the mixture was purified by rapid column chromatography (petroleum ether/ethyl acetate=70% to 100% for 20 min, then MeOH/DCM=0% to 10% for 40 min) to obtain Compound UB-181283a (993 mg, yield 66%). LCMS [M+H]⁺=438.9

### Step 2: UB-181283b

UB-181283a (993 mg, 2.3 mmol), DIEA (500 mg, 3.9 mmol) and 3-butyl p-toluenesulfonate (1.03g, 4.6 mmol) were dissolved in acetonitrile (80 mL) and the mixture was stirred at 80° C. for 18 hours. Then the mixture was purified by rapid column chromatography (petroleum ether/ethyl acetate=70% to 100% for 20 min, then MeOH/DCM=O % to 10% for 40 min) to obtain Compound UB-181283b (612 mg, yield 56%). LCMS [M+H]⁺=491.5.

### Step 3: UB-181283c

Compound UB-181283b (612 mg, 1.25 mmol), di-tert-butyl dicarbonate (320 mg, 1.48 mmol) and triethylamine (82 mg) were successively added to tetrahydrofuran (20 mL). The mixture was reacted at room temperature for 2 hours. Upon completion of the reaction, the reaction was poured into 10 mL of water and extracted with dichloromethane (5 mL*3). The organic phases were combined, then washed with saturated saline, dried over anhydrous Na₂SO₄, and concentrated by rotary evaporation under reduced pressure to obtain compound UB-181283c (552 mg, yield 75%) as colorless oil. LCMS [M+H]⁺=591.5

### Step 4: UB-181283d

Compound UB-181283c (30 mg, 0.050 mmol) and A3-I (38 mg, 0.103 mmol) were dissolved in DMF (10 mL), and added with dichlorobis(triphenylphosphonium)palladium (7.2 mg, 0.010 mmol), cuprous iodide (3.91 mg, 0.021 mmol) and triethylamine (150 mg, 1.49 mmol). The mixture was reacted at 80° C. overnight under nitrogen. The reaction solution was filtered through diatomite and the filtrate was concentrated to obtain crude product, which was purified by rapid chromatography (elution with DCM/MeOH=0%-20% for 30 min) to give product UB-181283d (10 mg, 24% yield). LCMS [M+H]⁺=832.4

### Step 5: UB-181283

Compound UB-181283d (10 mg, 0.012 mmol) and hydrochloric acid in dioxane (10 mL, 4 N) were added to tetrahydrofuran (10 mL) and reacted at room temperature for 2 h. Upon completion of the reaction, the reaction was concentrated by rotary evaporation under reduced pressure to give compound UB-181283 (8.8 mg, 100% yield). LCMS [M+H]⁺ 20=732.8. ¹H NMR (400 MHz, DMSO-d₆) δ11.98 (s, 1H), 11.00 (s, 1H), 9.78-9.69 (m, 2H), 9.68-9.47 (m, 2H), 8.80 (s, 1H), 8.46 (s, 1H), 8.16 (s, 1H), 7.87-7.66 (m, 5H), 7.75 (d, J=9.9 Hz, 2H), 7.66 (d, J=4.3 Hz, 4H), 7.63-7.49 (m, 2H), 7.32 (dd, J=100.9, 49.4 Hz, 4H) 7.18 (t, J=7.5 Hz, 1H), 5.12 (dd, J=13.2, 5.0 Hz, 1H), 4.40 (dd, J=5.8, 7.7 Hz, 2H), 3.86-3.66 (m, 3H), 3.22-3.02 (m, 4H), 2.96-2.80 (m, 3H), 2.66-2.60 (m, 1H), 2.39-2.18 (m, 3H), 2.01-1.93 (m, 3H).

### Synthesis of Compound UB-181237

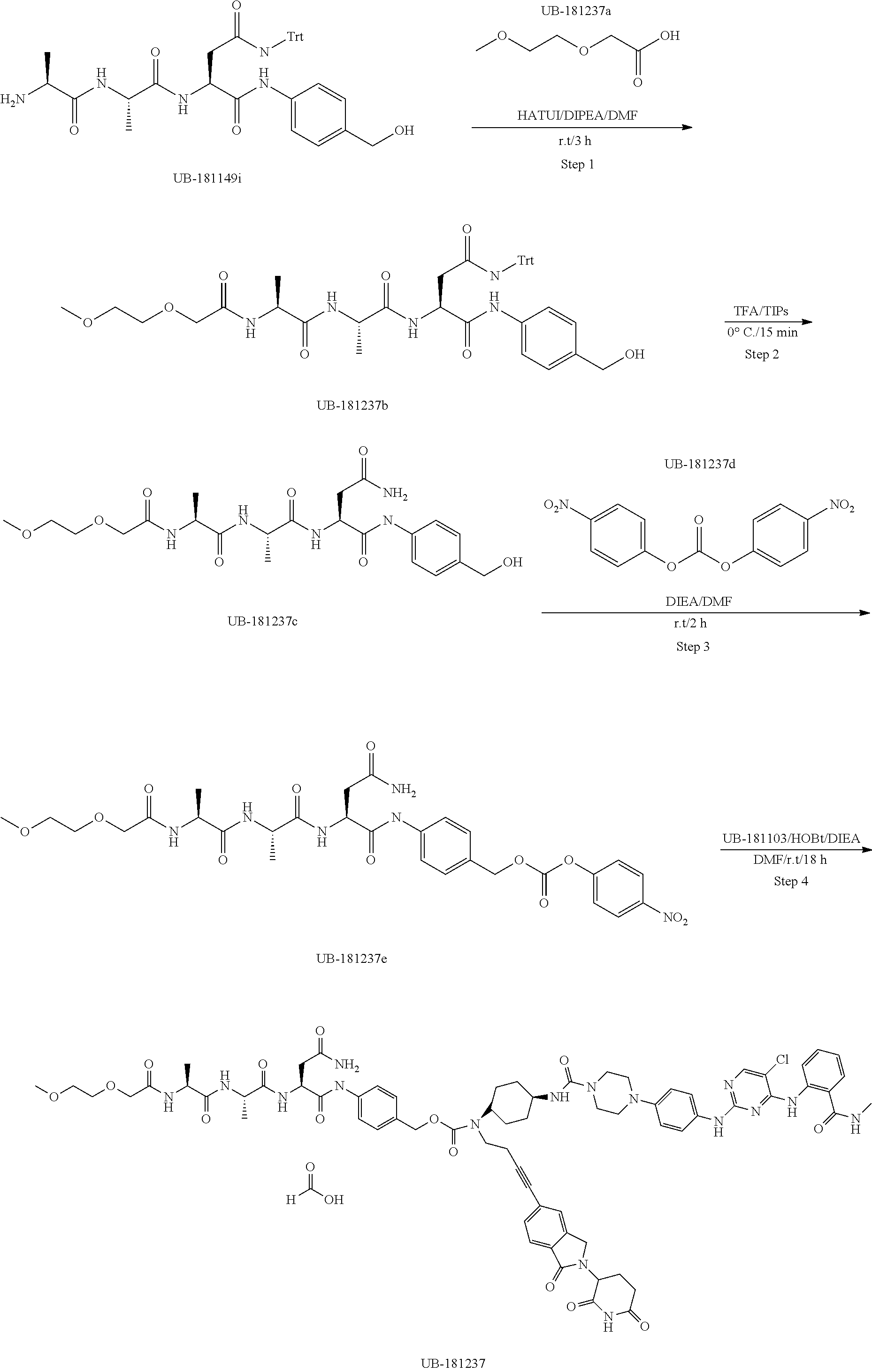

### Step 1: UB-181237b

To a solution of UB-181149i (2 g, 3.22 mmol), UB-181237a (430 mg, 3.2 mmol) and HATU (1.8 g, 4.73 mmol) in DMF (20 ml) was added DIEA (1.25 g, 9.66 mmol). The reaction solution was stirred at room temperature for 2 hours. The solid obtained by spin-drying of the reaction solution via an oil pump was purified by column chromatography (DCM/DCM:MeOH:THF (10:0.5:0.5)=0-96%) to give UB-181237b (750 mg, 40% yield) as white solid. LCMS [M+H]+=738.3

### Step 2: UB-181237c

To a solution of UB-181237b (700 mg, 0.95 mmol) in TIS (5 mL) was added CF3COOH (8 ml) and the reaction solution was stirred at 0° C. for 15 min. NaHCO3 (2.25 g, 25 ml aqueous solution) was added to the reaction solution. The mixture was filtered and the filtrate was purified by reversed-phase column chromatography (H2O: acetonitrile=0%-12%) to give UB-181237c (230 mg, 49% yield) as white solid. LCMS [M+H]+=496.6

### Step 3: UB-181237e

To a solution of UB-181237c (230 mg, 0.46 mmol) and UB-181237d (285 mg, 0.93 mmol) in DMF (5 ml) was added DIEA (192 mg, 1.4 mmol). The reaction was reacted at room temperature for 2. The reaction solution was pulled dry by an oil pump, and the resulting solid was slurried with ethyl ether. The mixture was purified by preparative TLC (DCM/MeOH=10/1) to give UB-181237e (230 mg, 49% yield) as yellow solid. LCMS [M+H]⁺=661.5

### Step 4: UB-181237

To a solution of UB-181237e (230 mg, 0.34 mmol), UB-181103 (316 mg, 0.34 mmol), and HOBt (94 mg, 0.7 mmol) in DMF (2 mL) was added DIPEA (135 mg, 1.1 mmol) and stirred for 18 h at room temperature. The reaction solution was purified by preparative HPLC to obtain UB-181237 (98 mg, 21% yield) as white solid. LCMS [M+H]+=1394.0.

### Synthesis of Compound UB-181238

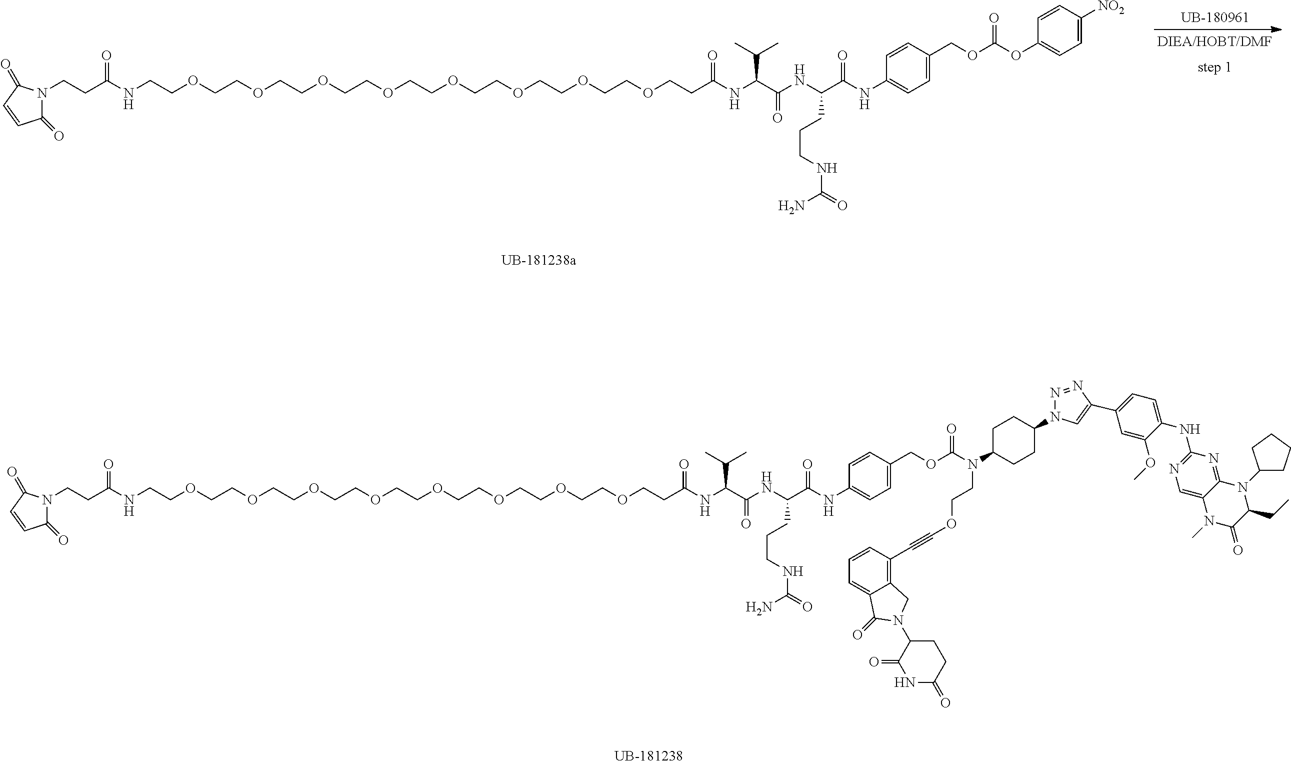 [SMILES: CC(C)[C@H](NC(=O)CCOCCOCCOCCOCCOCCOCCOCCOCCNC(=O)CCN1C(=O)C=CC1=O)C(=O)N[C@@H](CCCNC(N)=O)C(=O)Nc1ccc(COC(=O)Oc2ccc([N+](=O)[O-])cc2)cc1 ; CC[C@H]1C(=O)N(C)c2cnc(Nc3ccc(-c4cn([C@H]5CC[C@@H](N(CCOC#Cc6cccc7c6CN(C6CCC(=O)NC6=O)C7=O)C(=O)OCc6ccc(NC(=O)[C@H](CCCNC(N)=O)NC(=O)[C@@H](NC(=O)CCOCCOCCOCCOCCOCCOCCOCCOCCNC(=O)CCN7C(=O)C=CC7=O)C(C)C)cc6)CC5)nn4)cc3OC)nc2N1C1CCCC1]

### Step 1: UB-181238

To a solution of UB-181238a (49 mg, 0.04 mmol) in DMF (3 mL) was added UB-180961 (42 mg, 0.04 mmol), HOBT (5.9 mg, 0.04 mmol), and DIEA (11.3 mg, 0.09 mmol), and stirred at room temperature for 16 hours under N2 protection. The solution was concentrated and purified by preparative TLC to give (12.8 mg, 99% purity) yellow solid as pure product. LCMS [M+H]⁺=1866.0

### Synthesis of Compound UB-181241d

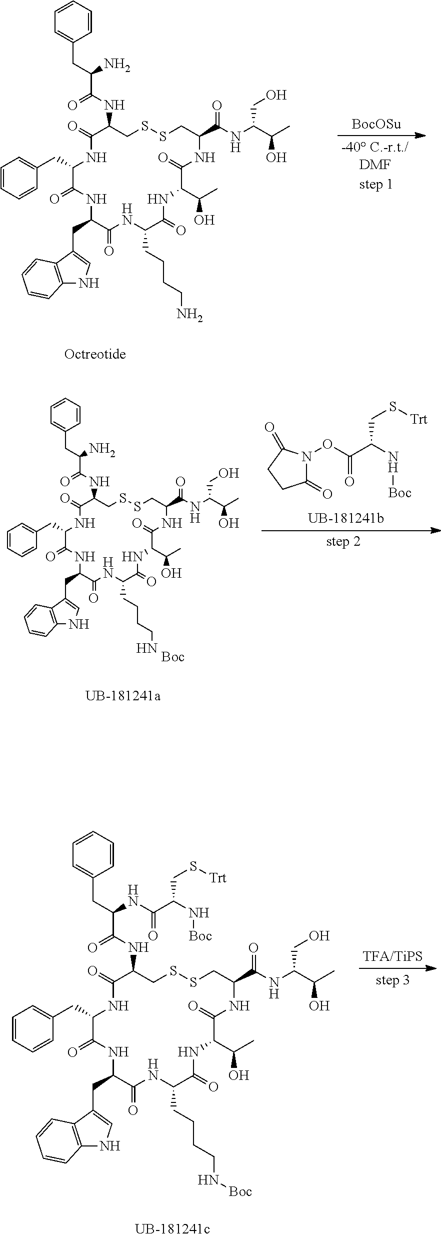

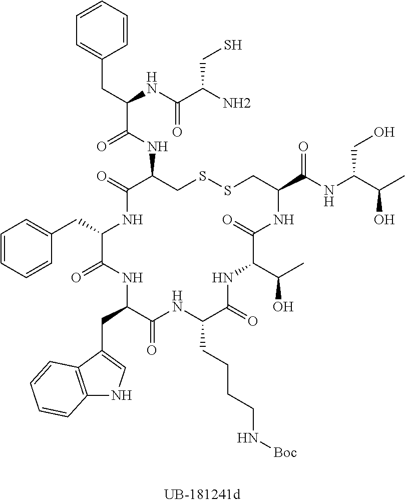 [SMILES: C[C@@H](O)[C@@H]1NC(=O)[C@H](CCCCNC(=O)OC(C)(C)C)NC(=O)[C@@H](Cc2c[nH]c3ccccc23)NC(=O)[C@H](Cc2ccccc2)NC(=O)[C@@H](NC(=O)[C@@H](Cc2ccccc2)NC(=O)[C@@H](N)CS)CSSC[C@@H](C(=O)N[C@H](CO)[C@@H](C)O)NC1=O]

### Step 1: UB-181241a

Octreotide (200 mg, 0.19 mmol) and DIEA (48 mg, 0.37 mmol) were dissolved in DMF (5 mL) and cooled to −40° C. Then BocOSu (40 mg, 0.19 mmol) was added and the mixture was stirred under nitrogen protection at room temperature for 2 hours. The reaction solution was concentrated and then subjected to reverse phase column chromatography to obtain target product UB-181241a (200 mg, yield 91%) as white solid. LCMS [M+H]⁺=1120.0

### Step 2: UB-181241c

Compound UB-181241a (200 mg, 0.18 mmol) was dissolved in DMF (5 mL), then UB-181241b (100 mg, 0.18 mmol) and DIEA (35 mg, 0.27 mmol) were added, and the mixture was stirred at room temperature overnight. The reaction solution was subjected to reverse phase column chromatography to obtain target product UB-181241c (130 mg, yield 47%) as white solid. LCMS [M+H]⁺=1565.5

### Step 3: UB-181241d

Compound UB-181241c (930 mg, 0.10 mmol) was dissolved in TFA (3 mL) and a catalytic amount of iPr3SiH was added and the mixture was stirred at room temperature for 10 min. The reaction solution was concentrated at low temperature and then pulped with isopropyl ether, and then the solid was filtered out and dried to give target product UB-181241d (880 mg, 100% yield) as white solid. LCMS [M+H]⁺=1123.2.

### Synthesis of Compound UB-181242

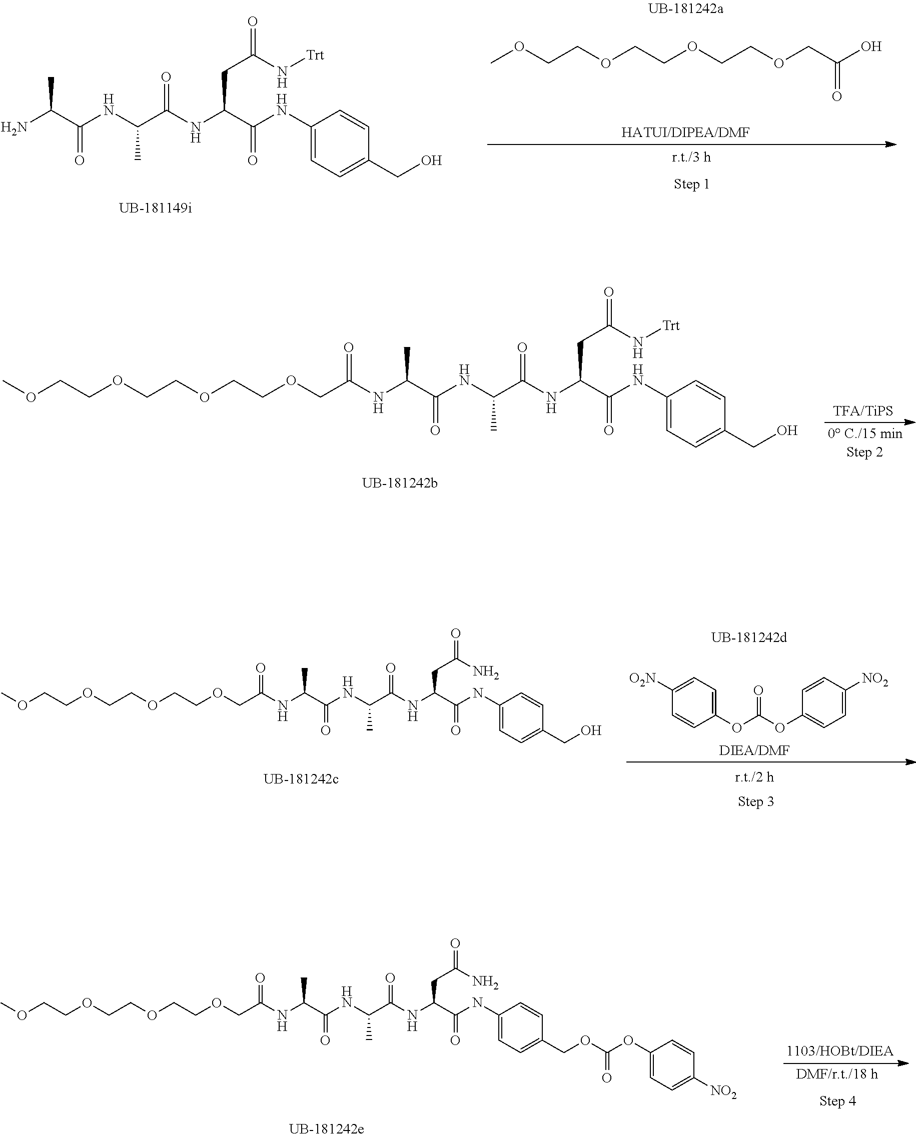

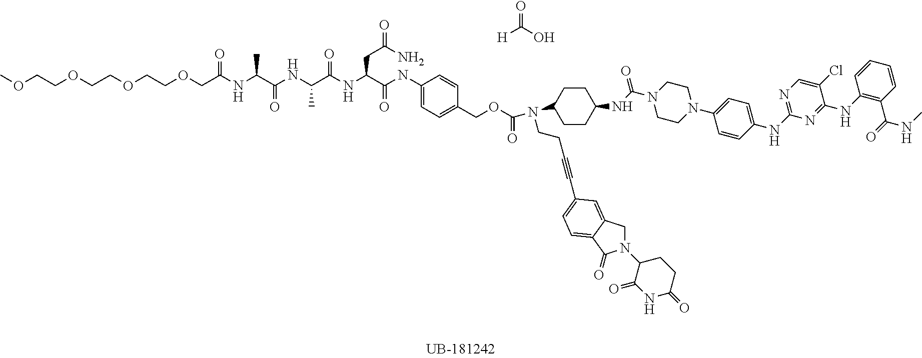

### Step 1: UB-181242b

To a solution of UB-181149i (559 mg, 0.9 mmol), UB-181242a (200 mg, 0.9 mmol) and HATU (513 mg, 1.35 mmol) in DMF (5 ml) was added DIEA (350 mg, 2.7 mmol). The reaction solution was stirred at room temperature for 2 hours. The solid obtained by pulled drying of the reaction solution via an oil pump was purified by column chromatography (DCM/DCM:MeOH:THF (10:0.5:0.5)=0-96%) to give UB-181242b (380 mg, 40% yield) as white solid. LCMS: [M+H]+=826.7

### Step 2: UB-181242c

To a solution of UB-181242b (360 mg, 0.44 mmol) in TIS (1.5 mL) was added CF3COOH (3 ml) and the reaction solution was stirred at 0° C. for 15 min. NaHCO3 (2.25 g, 25 ml aqueous solution) was added to the reaction solution. The mixture was filtered and the filtrate was purified by reversed-phase column chromatography (H2O:acetonitrile=0%-12%) to give UB-181242c (80 mg, 31% yield) as white solid. LCMS [M+H]+=584.6

### Step 9: UB-181242e

To a solution of UB-181242c (80 mg, 0.14 mmol) and UB-181242d (84 mg, 0.28 mmol) in DMF (5 ml) was added DIEA (60 mg, 0.41 mmol). The mixture was reacted at room temperature for 2 h. The reaction solution was pulled dry by an oil pump, and the resulting solid was pulped with ether. The mixture was purified by preparative TLC (DCM/MeOH=10/1) to give UB-181242e (70 mg, 60% yield) as yellow solid. LCMS [M+H]+=749.5

### Step 10: UB-181242

To a solution of UB-181242e (20 mg, 0.03 mmol), 1103 (24.3 mg, 0.03 mmol), and HOBt (7.2 mg, 0.06 mmol) in DMF (1 mL) was added DIPEA (10.3 mg, 0.09 mmol) and stirred at room temperature for 18 h. The reaction solution was purified by preparative HPLC to obtain UB-181242 (9 mg, 23% yield) as white solid. LCMS [M+H]+=1482.1

### Synthesis of Compound UB-181243

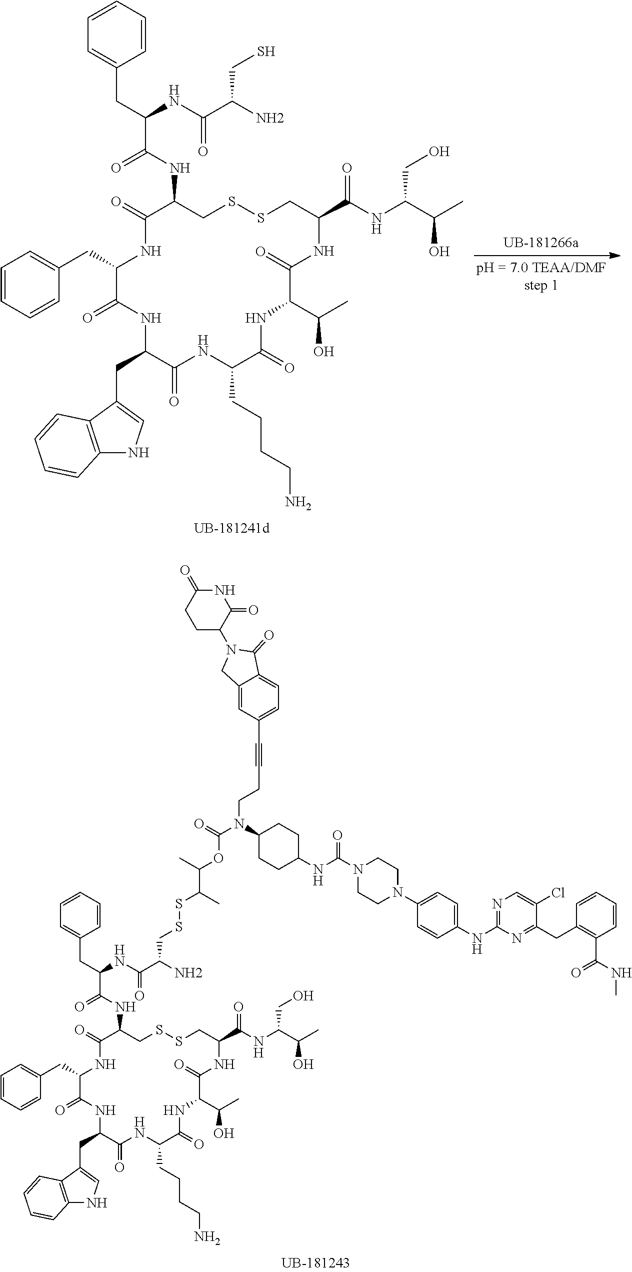

### Step 1: UB-181243

Compound UB-181241d (150 mg, 0.13 mmol) was dissolved in 1M TEAA (2 mL), and then added with UB-181266a (74 mg, 0.07 mmol) in DMF (1 mL). The reaction solution was stirred at room temperature for 2 hours and then separated by reverse phase column chromatography to give 100 mg of crude product. The crude product was prepared to obtain target product UB-181243 (50 mg, yield 18%) as white solid. LCMS [M/2+H]⁺=1064.2

### Synthesis of Compound UB-181246

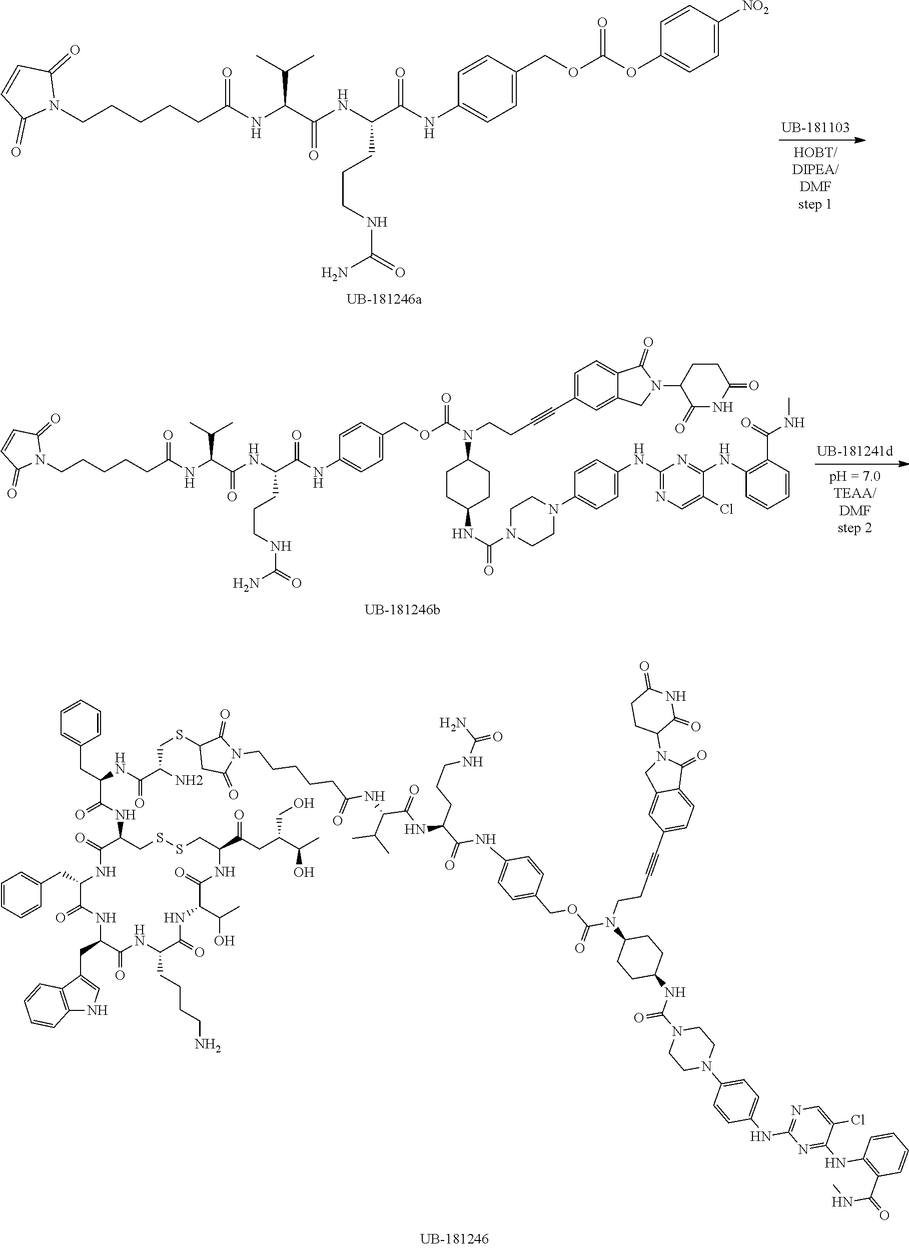

### Step 1: UB-181246b

Compound UB-181246a (60 mg, 0.08 mmol) was dissolved in DMF (1 mL), and then UB-11103 (71 mg, 0.0.08 mmol), HOBT (22 mg, 0.16 mmol) and DIEA (32 mg, 0.24 mmol) were added. After reacting at room temperature overnight, the mixture was subjected to preparative chromatography to obtain target product UB-181246b (20 mg, yield 17%) as white solid. LCMS [M+H]⁺=1472.4

### Step 2: UB-181246

Compound UB-181241d (16 mg, 0.01 mmol) was dissolved in DMF (1 mL) and DIEA (0.3 mL), then UB-181246b (21 mg, 0.01 mmol) was added, and the mixture was reacted at room temperature overnight. The reaction solution was subjected to preparative chromatography to obtain target product UB-181243 (4.5 mg, yield 12%) as white solid. LCMS [M/2+H]⁺=1298.4

### Synthesis of Compound UB-181247

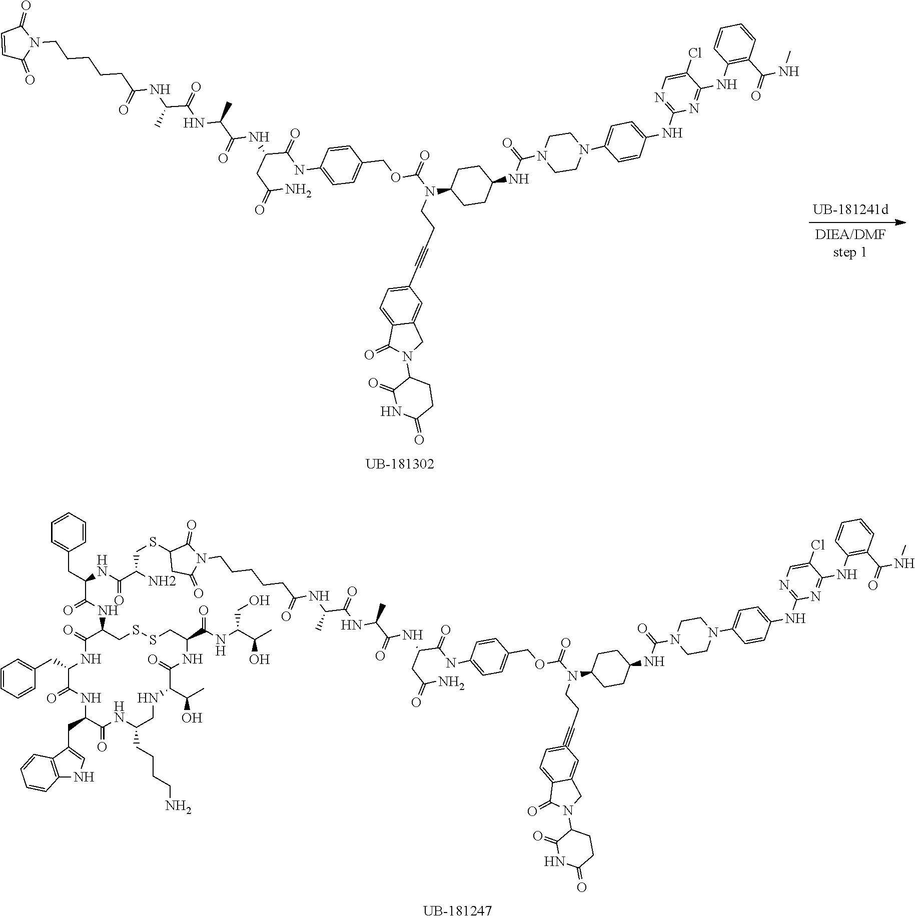

### Step 1: UB-181247

Compound UB-181241d (16 mg, 0.01 mmol) was dissolved in DMF (1 mL) and DIEA (0.3 mL), and then UB-181302 (121 mg, 0.01 mmol) was added. After reacting at room temperature overnight, the mixture was subjected to preparative chromatography to obtain target product UB-181247 (5.1 mg, yield 18%) as white solid. LCMS [M/2+H]⁺=1297.6

### Synthesis of Compound UB-181263

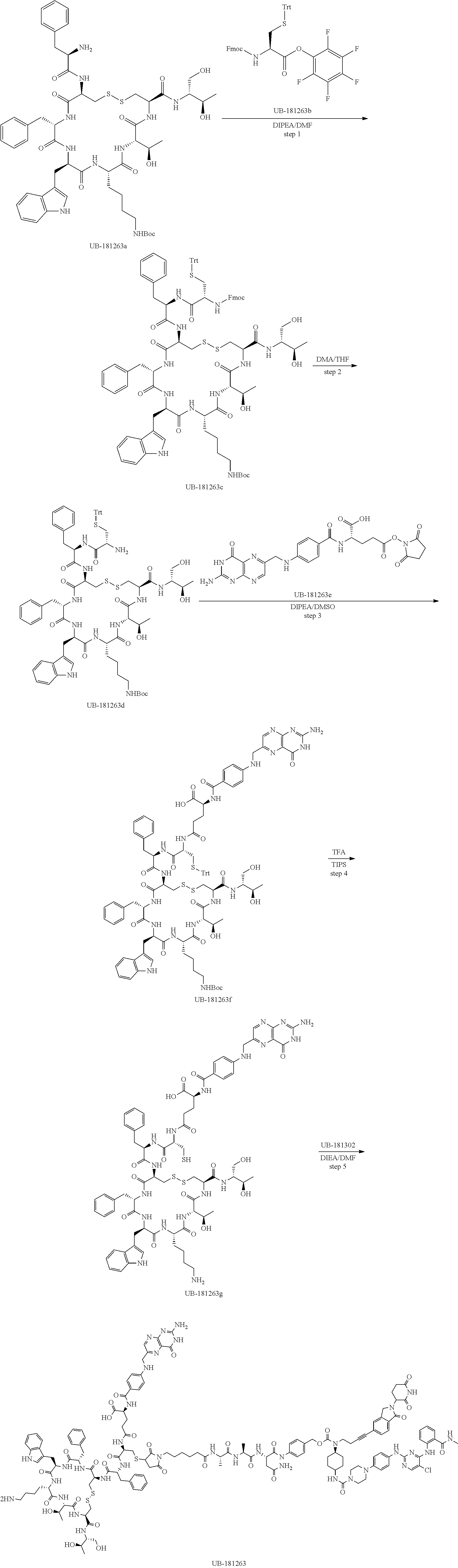

### Step 1: UB-181263c

Compound UB-181263a (160 mg, 0.14 mmol) was dissolved in DMF (2 mL), and then UB-181263b (107 mg, 0.13 mmol) and DIEA (28 mg, 0.21 mmol) were added. After reacting at room temperature for 2 hours, the mixture was subjected to reverse phase column chromatography to obtain target product UB-181263c (100 mg, yield 41%) as white solid. LCMS [M+H]⁺=1687.3

### Step 2: UB-181263d

Compound UB-181263c (20 mg, 0.01 mmol) was dissolved in THF (3 mL), and then added with DMA/THF (5 mL). The mixture was reacted at room temperature for 2 hours. The reaction solution was concentrated at low temperature to obtain crude product. This crude product was pulped with diethyl ether to obtain target product UB-181263d (20 mg, yield 100%) as white solid. LCMS [M+H]⁺=1465.8

### Step 3: UB-181263f

Compound UB-181263d (100 mg, 0.07 mmol) was dissolved in DMF (2 mL), and then added with UB-181263e (110 mg, 0.20 mmol) and DIEA (13 mg, 0.10 mmol). The mixture was reacted at room temperature overnight. The reaction solution was separated by reverse phase column chromatography to obtain target product UB-181263f (80 mg, yield 62%) as yellow solid. LCMS [M+H]⁺=1890.2

### Step 4: UB-181263g

Compound UB-181263f (30 mg, 0.02 mmol) was dissolved in TFA (2 mL) and a catalytic amount of iPr₂SiH was added. After reacting at room temperature for 10 minutes, the mixture was concentrated to obtain target crude product UB-181263g (30 mg, yield 100%) as yellow solid. LCMS [M+H]⁺=1547.5

### Step 5: UB-181263

Compound UB-181263g (15 mg, 0.01 mmol) was dissolved in DMF (2 mL) and DIEA (0.3 mL), and then UB-181302 (14 mg, 0.01 mmol) was added. After reacting at room temperature overnight, the mixture was subjected to preparative chromatography to obtain target product UB-181263g (2.8 mg, yield 10%) as light yellow solid. LCMS [M/2+H]⁺=1510.2

### Synthesis of Compound UB-181265

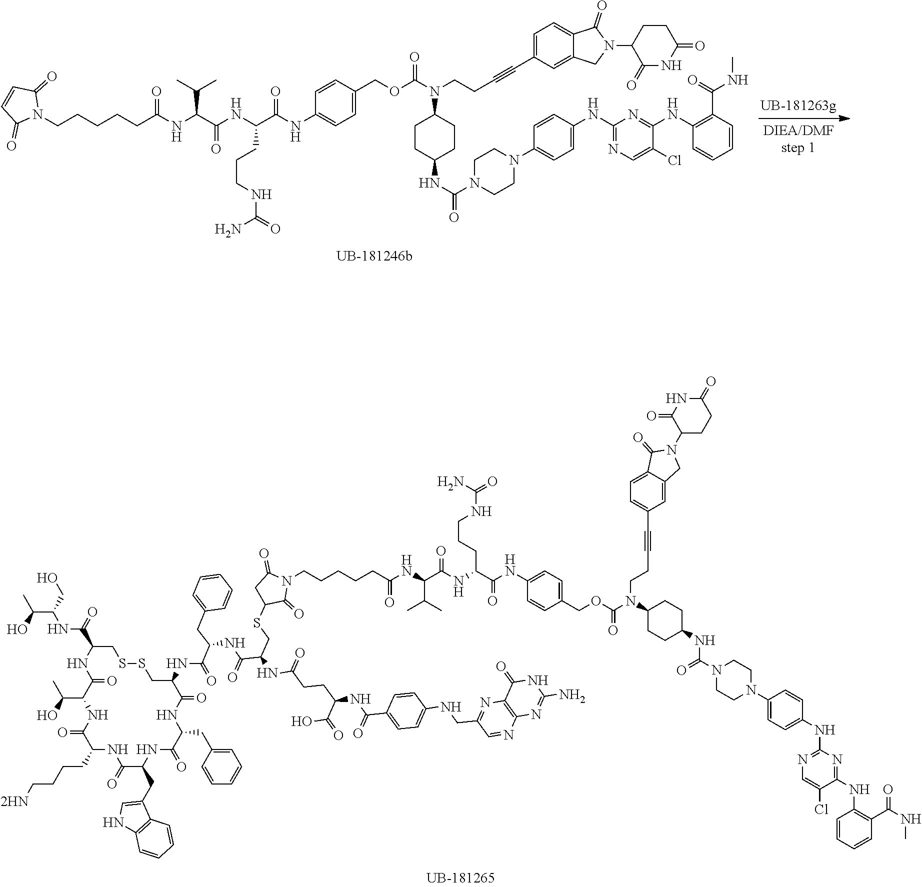

### Step 1: UB-181265

Compound UB-181246b (20 mg, 0.01 mmol) was dissolved in DMF (2 mL) and DIEA (0.3 mL), and then UB-181263g (19 mg, 0.01 mmol) was added. After reacting at room temperature overnight, the mixture was subjected to preparative chromatography to obtain target product UB-181243 (3.1 mg, yield 8%) as light yellow solid. LCMS [M/2+H]⁺=1511.4

### Synthesis of Compound UB-181266 & 181267

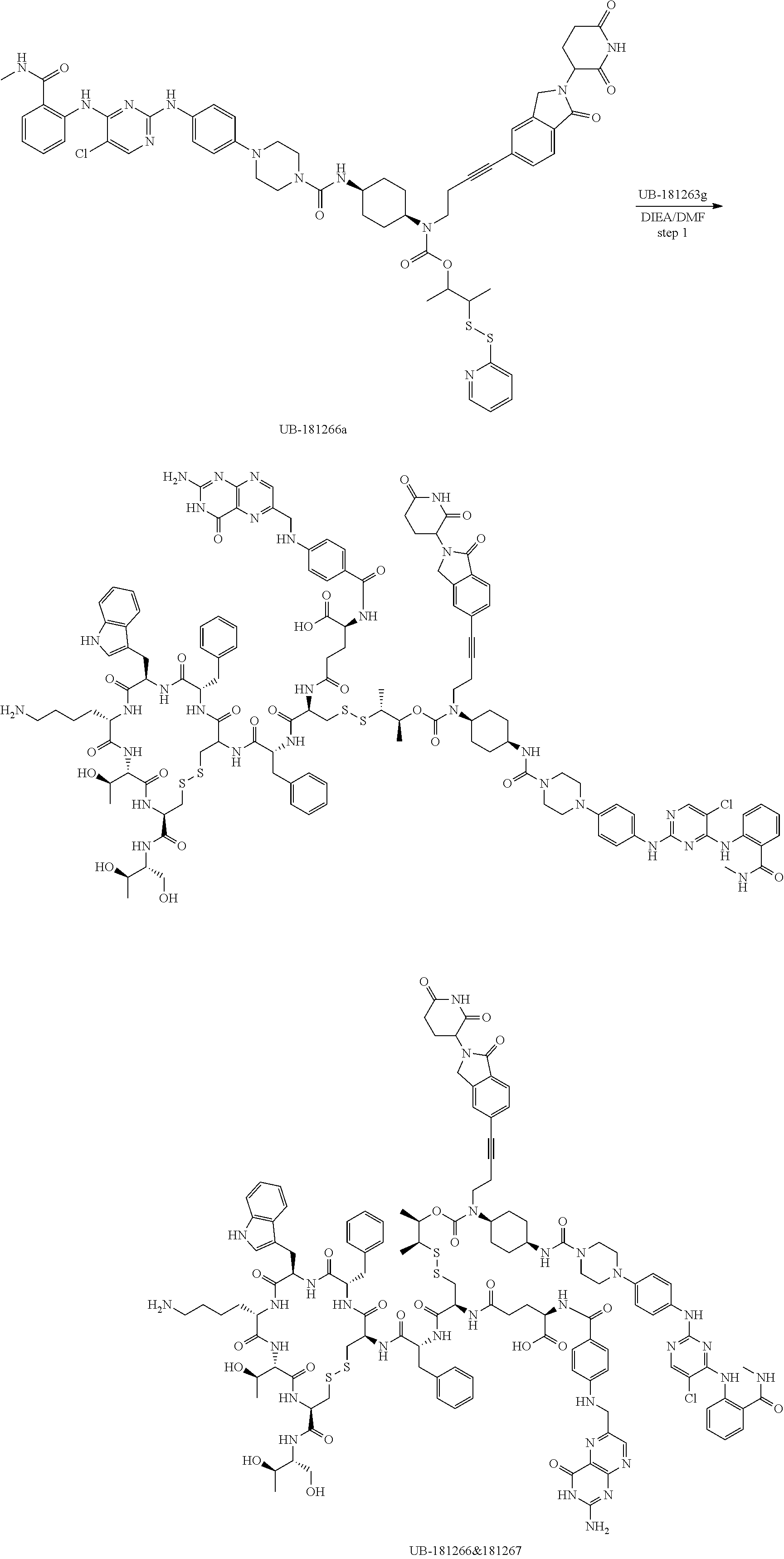

### Step 1: UB-181266 & 181267

Compound UB-181266a was synthesised in a method similar to UB-181326. Compound UB-181266a (20 mg, 0.01 mmol) was dissolved in DMF (2 mL) and DIEA (0.2 mL), and then UB-181263g (14 mg, 0.01 mmol) was added. After reacting at room temperature overnight, the mixture was subjected to preparative chromatography to obtain target product UB-181266 (1.9 mg, yield 4%) as light yellow solid and target product UB-181267 (1.6 mg, yield 3%) as light yellow solid. LCMS [M/2+H]⁺=1275.6

### Synthesis of Compound UB-181268

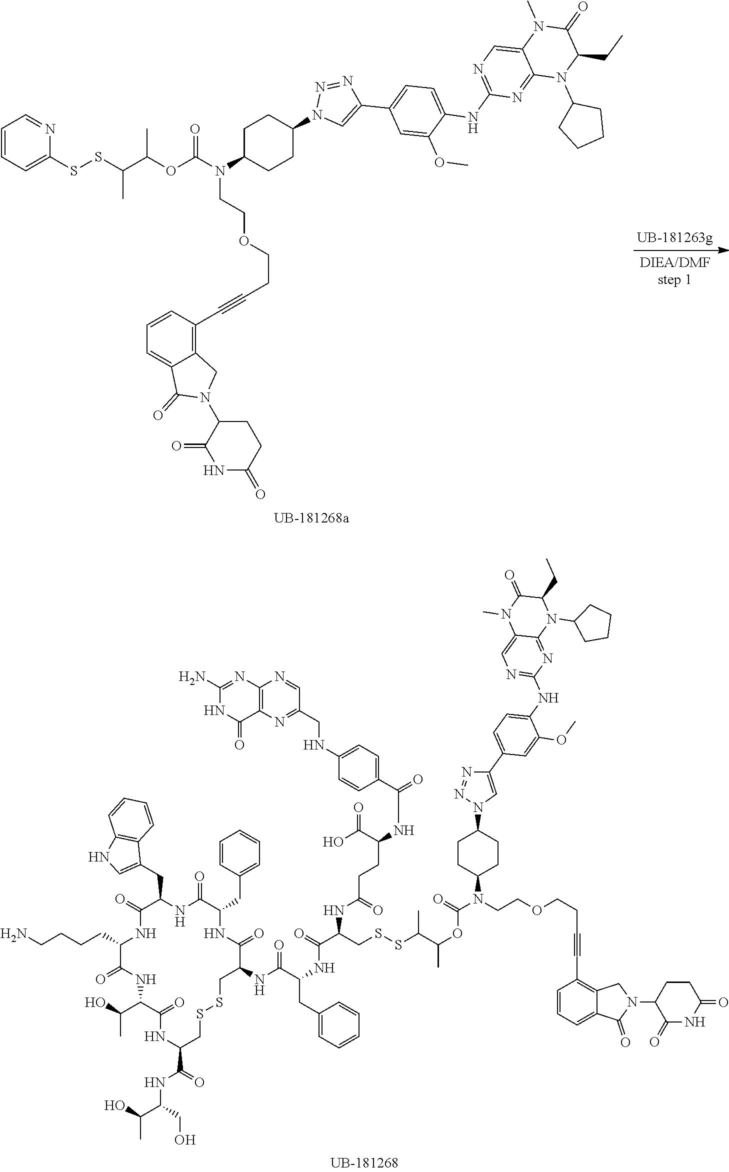

### Step 1: UB-181268

The synthesis of UB-181268a was the same as that of Compound UB-181325. Compound UB-181268a (20 mg, 0.01 mmol) was dissolved in DMF (2 mL) and DIEA (0.2 mL), and then UB-181243g (14 mg, 0.01 mmol) was added. After reacting at room temperature overnight, the mixture was subjected to preparative chromatography to obtain target product UB-181268 (5.9 mg, yield 13%) as white solid. LCMS [M/2+H]⁺=1281.2.

### Synthesis of Compound UB-181275

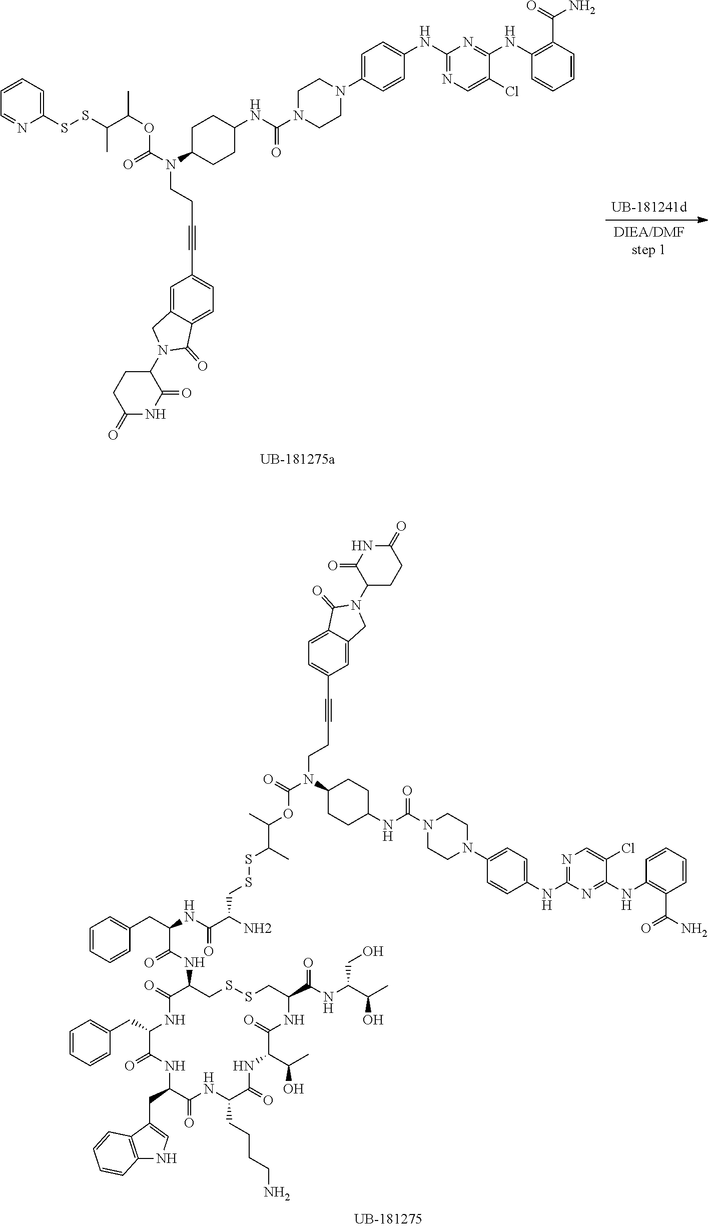

### Step 1: UB-181275

Compound UB-181275a was synthesized in a method similar to UB-181325. Compound UB-181275a (20 mg, 0.02 mmol) was dissolved in DMF (1 mL) and DIEA (0.3 mL), and then UB-181241d (20 mg, 0.02 mmol) was added. After reacting at room temperature overnight, the mixture was subjected to preparative chromatography to obtain target product UB-181275 (6.6 mg, yield 17%) as white solid. LCMS [M/2+H]⁺=1056.8

### Synthesis of Compound UB-181280

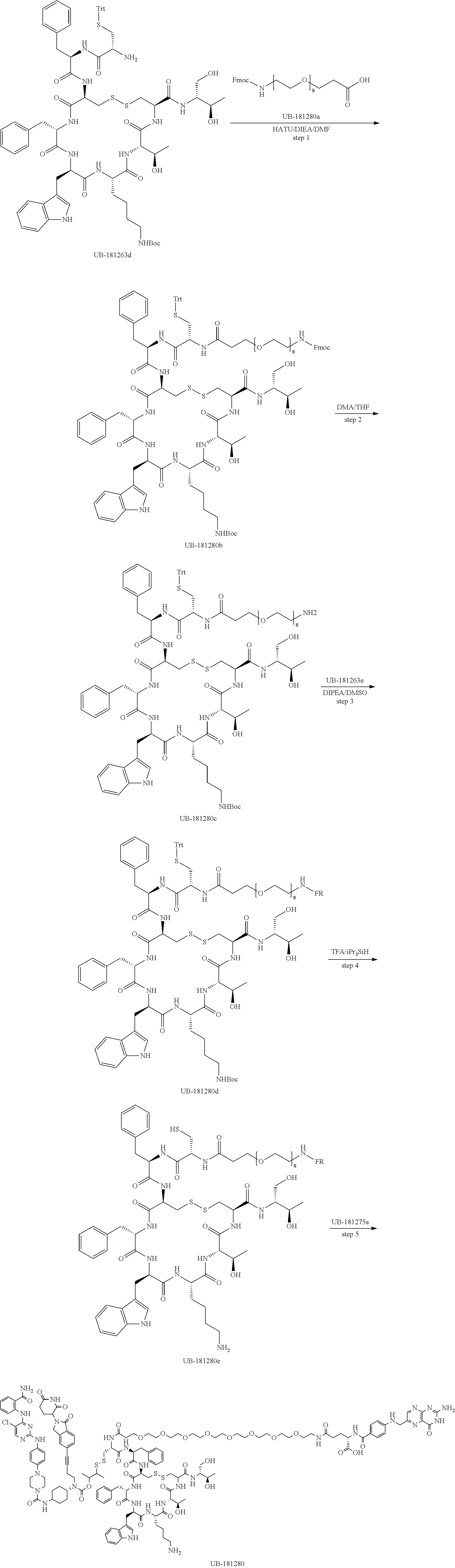

### Step 1: UB-181280b

Compound UB-181280a (272 mg, 0.41 mmol) was dissolved in DMF (5 mL), and then HATU (234 mg, 0.62 mmol) and DIEA (158 mg, 1.23 mmol) were added. After reacting at room temperature for 1 hour, UB-181263d (600 mg, 0.41 mmol) was added, and the mixture continued to react at room temperature for 2 hours. The reaction solution was concentrated and then separated by reverse phase column chromatography to obtain target product UB-181280b (550 mg, yield 64%) as yellow solid. LCMS [M/2+H]⁺=1056.9

### Step 2: UB-181280c

Compound UB-181280b (560 mg, 0.27 mmol) was dissolved in THF (3 mL), and then added with DMA/THF (6 mL), and the mixture was reacted at room temperature for 2 hours. The reaction solution was concentrated and then pulped with diethyl ether to obtain target product UB-181280c (450 mg, yield 90%) as yellow solid. LCMS [M+H]⁺=1889.3

### Step 3: UB-181280d

Compound UB-181280c (500 mg, 0.26 mmol) was dissolved in DMF (3 mL), and then added with UB-181263g (427 mg, 0.79 mmol) and DIEA (51 mg, 0.40 mmol). After reacting at room temperature overnight, the mixture was subjected to reverse phase column chromatography to obtain target product UB-181280d (80 mg, yield 13%) as yellow solid. LCMS [M/2+H]⁺=1158.1

### Step 4: UB-181280e

Compound UB-181280d (60 mg, 0.03 mmol) was dissolved in TFA (2 mL) and a catalytic amount of iPr₂SiH was added, and the mixture was reaction at room temperature for 10 min. The reaction solution was concentrated at low temperature and then separated by reverse phase column chromatography to obtain target product UB-181280e (40 mg, yield 78%) as yellow solid. LCMS [M/2+H]⁺=986.1

### Step 5: UB-181280

Compound UB-181280e (20 mg, 0.01 mmol) was dissolved in DMF (1 mL) and DIEA (0.3 mL), and then UB-181275a (11 mg, 0.01 mmol) was added. After reacting at room temperature overnight, the mixture was subjected to preparative chromatography to obtain target product UB-181280 (2 mg, yield 7%) as light yellow solid. LCMS [M/3+H]⁺=987.6

### Synthesis of Compound UB-181285

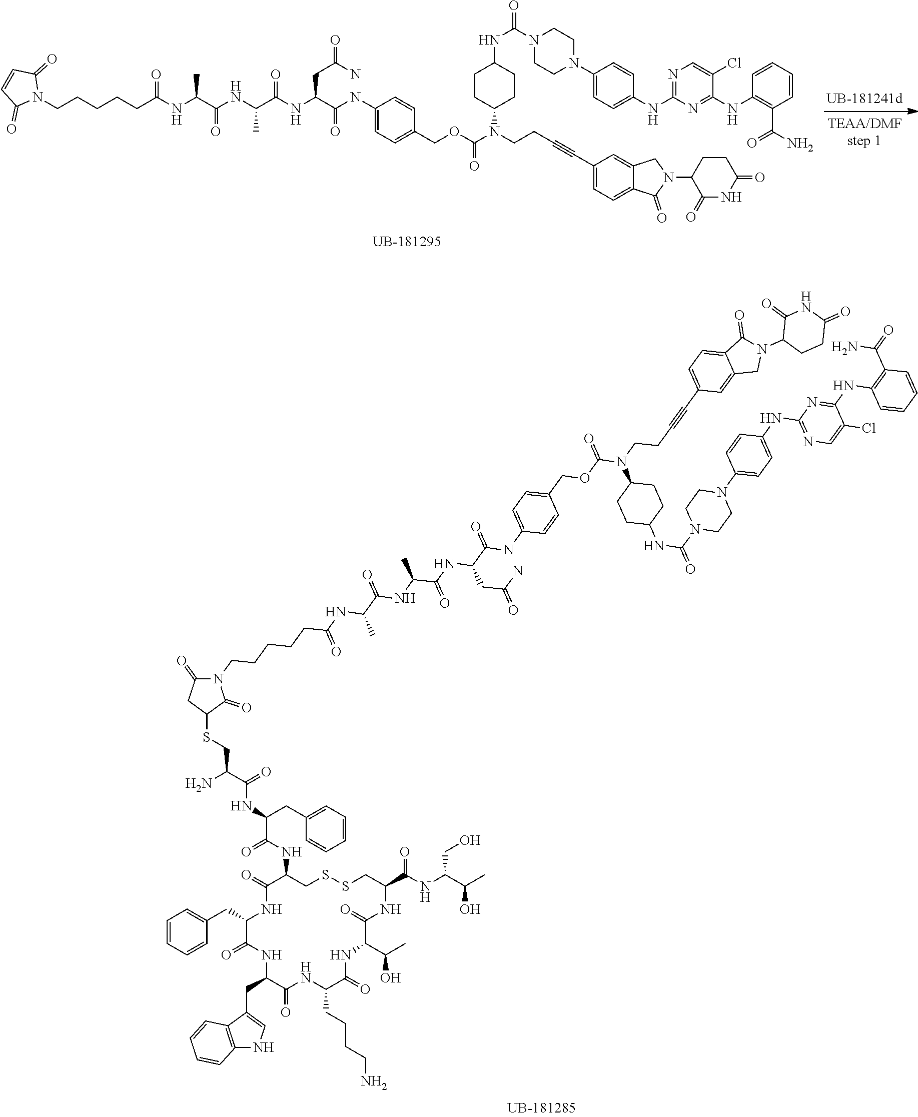

### Step 1: UB-181285

Compound UB-181241d (25 mg, 0.02 mmol) was dissolved in 1M TEAA (1 mL), and then added with UB-181295 (32 mg, 0.02 mmol) in DMF (1 mL). After reacting at room temperature for 2 hours, the mixture was subjected to preparative chromatography to obtain target product UB-181285 (2.1 mg, yield 4%) as white solid. LCMS [M/3+H]⁺=861.4

### Synthesis of Compound UB-181289

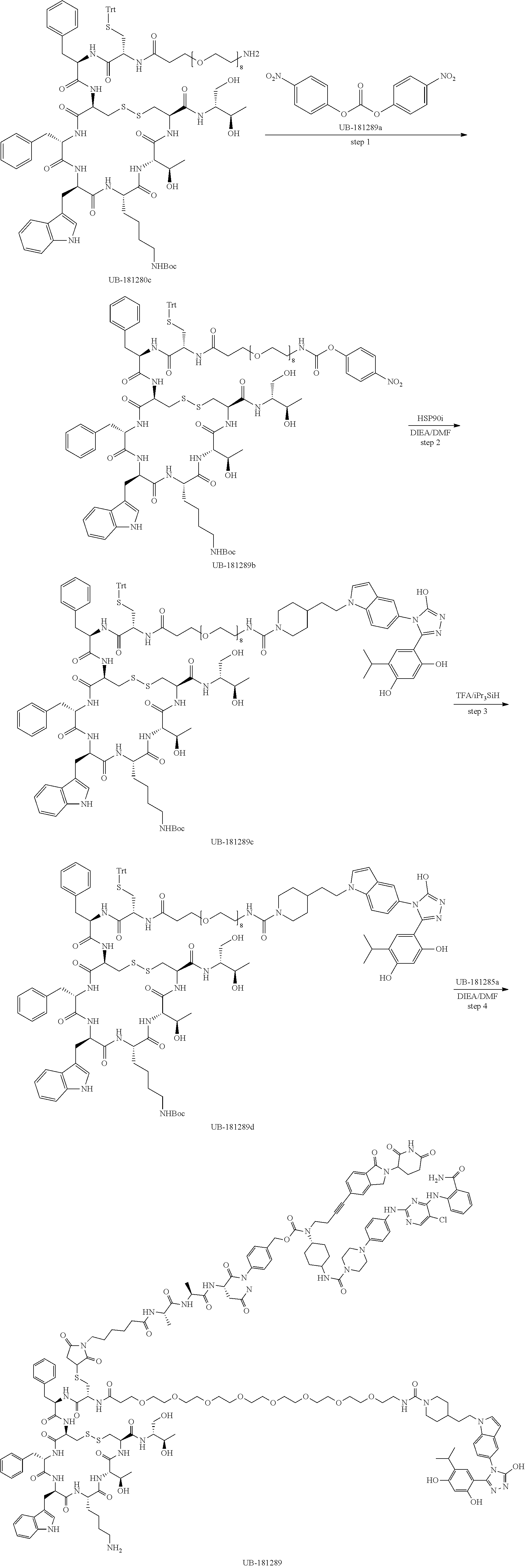

### Step 1: UB-181289b

Compound UB-181280c (200 mg, 0.11 mmol) and UB-181289a (48 mg, 0.16 mmol) were dissolved in pyridine (30 mL), and then the mixture was reacted at room temperature overnight. The reaction solution was subjected to reverse phase column chromatography to obtain target product UB-181289b (160 mg, yield 74%) as yellow solid. LCMS [M-Trt-Boc+H]⁺=1711.8

### Step 2: UB-181289c

Compound UB-181289b (140 mg, 0.07 mmol) was dissolved in DMF (2 mL) and then added with DIEA (0.3 mL) and HSP-90 (31 mg, 0.07 mmol), and the mixture was reacted at room temperature for 30 minutes. The reaction solution was separated by reverse phase column chromatography to obtain target product UB-181280c (100 mg, yield 62%) as white solid. LCMS [M/2+H]⁺=1189.8

### Step 3: UB-181289d

Compound UB-181280c (50 mg, 0.02 mmol) was dissolved in TFA (0.8 mL) and a catalytic amount of iPr₃SiH was added, and the mixture was reacted at room temperature for 10 min. The reaction solution was separated by reverse phase column chromatography to obtain target product UB-181280d (30 mg, yield 70%) as white solid. LCMS [M+H]⁺=1018.9

### Step 5: UB-181289

Compound UB-181280d (20 mg, 0.01 mmol) was dissolved in DMF (1 mL) and DIEA (0.3 mL), and then UB-181285a (14 mg, 0.01 mmol) was added. After reacting at room temperature overnight, the mixture was subjected to preparative chromatography to obtain target product UB-181289 (1.9 mg, yield 6%) as white solid. LCMS [M/3+H]⁺=1165.2

### Synthesis of Compound UB-181290

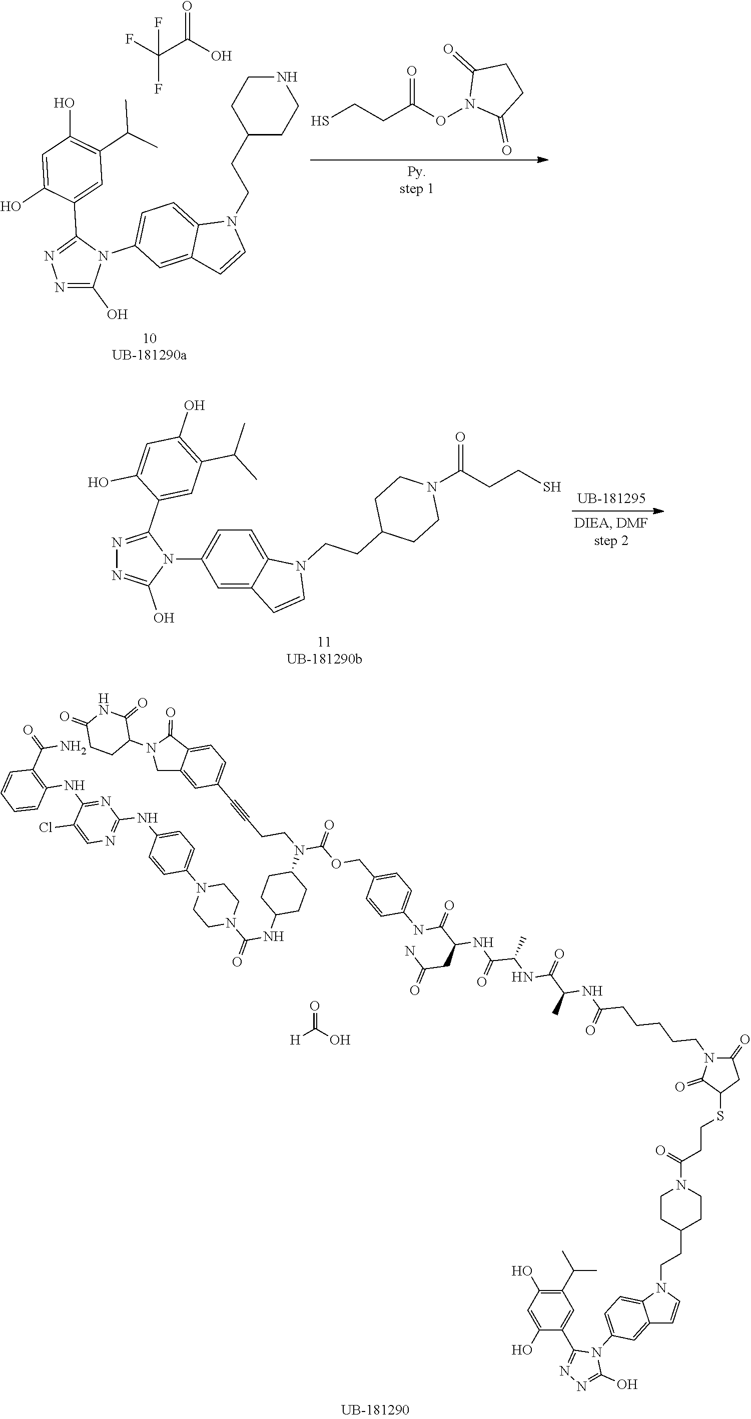

### Step 1: UB-181290b

UB-181290b (100 mg, 0.17 mmol), and 2,5-dioxopyrrolidin-1-yl 3-mercaptopropionate (40 mg, 0.21 mmol) were dissolved in pyridine (1 mL). The mixture was reacted at room temperature overnight. The reaction solution was spin-dried, and the residue was subjected to thin layer chromatography (DCM/MeOH=15/1) to obtain UB-181290c (60 mg, 64% yield) as white solid. LCMS [M+H]⁺=550.8. ¹H NMR (400 MHz, DMSO) δ 11.87 (s, 1H), 9.54 (d, J=21.5 Hz, 2H), 7.50-7.40 (m, 3H), 6.93 (dd, J=8.6, 1.9 Hz, 1H), 6.68 (s, 1H), 6.43 (d, J=2.6 Hz, 1H), 6.24 (s, 1H), 4.33 (s, 1H), 4.21 (d, J=6.8 Hz, 2H), 3.80 (d, J=11.4 Hz, 1H), 2.96-2.83 (m, 3H), 2.64 (dd, J=29.0, 5.1 Hz, 4H), 2.33 (d, J=1.8 Hz, 2H), 1.70 (dd, J=19.0, 11.0 Hz, 4H), 1.44 (s, 1H), 0.79 (d, J=6.9 Hz, 6H).

### Step 10: UB-181290

UB-181290b (11 mg, 0.02 mmol), UB-181295 (30 mg, 0.02 mmol) and DIEA (5 mg, 0.04 mmol) were dissolved in DMF (1 mL), and the mixture was reacted at room temperature for 1 hour. The reaction solution was purified by high pressure preparation (MeCN/H2O/FA) to give UB-181290 (2.3 mg, 7.6% yield) as yellow solid. LCMS[M/2+H]=1005.17. ¹H NMR (400 MHz, DMSO) δ 11.86 (d, J=15.4 Hz, 2H), 10.98 (s, 1H), 9.70 (s, 1H), 9.55 (d, J=23.7 Hz, 2H), 9.22 (s, 1H), 8.79 (s, 1H), 8.28 (s, 2H), 8.16 (d, J=6.2 Hz, 2H), 8.11 (d, J=7.6 Hz, 1H), 8.01 (d, J=7.0 Hz, 1H), 7.79 (s, 1H), 7.73 (s, 1H), 7.67 (m, J=16.0, 8.2 Hz, 3H), 7.60 (s, 1H), 7.52-7.43 (m, 6H), 7.42 (d, J=1.8 Hz, 1H), 7.40 (s, 1H), 7.31 (d, J=8.2 Hz, 2H), 7.08 (s, 1H), 6.98-6.87 (m, 4H), 6.67 (s, 1H), 6.42 (d, J=2.9 Hz, 1H), 6.24 (s, 1H), 6.12 (d, J=5.5 Hz, 1H), 5.08 (m, J=15.0, 6.5 Hz, 3H), 4.61 (d, J=7.2 Hz, 1H), 4.41 (d, J=17.7 Hz, 1H), 4.28 (m, J=16.4, 9.7 Hz, 3H), 4.20 (m, J=12.5, 6.7 Hz, 3H), 4.00 (d, J=6.9 Hz, 1H), 3.78 (s, 3H), 3.48 (s, 5H), 3.17 (m, J=18.4, 9.0 Hz, 2H), 3.03 (s, 4H), 2.97-2.78 (m, 6H), 2.76-2.56 (m, 10H), 2.45-2.31 (m, 2H), 2.09 (t, J=7.2 Hz, 2H), 2.04-1.95 (m, 1H), 1.82 (d, J=10.6 Hz, 4H), 1.69 (m, J=18.7, 10.9 Hz, 4H), 1.47 (s, 9H), 1.21 (t, J=7.4 Hz, 9H), 0.81 (t, J=15.8 Hz, 6H).

### Synthesis of Compound UB-181291

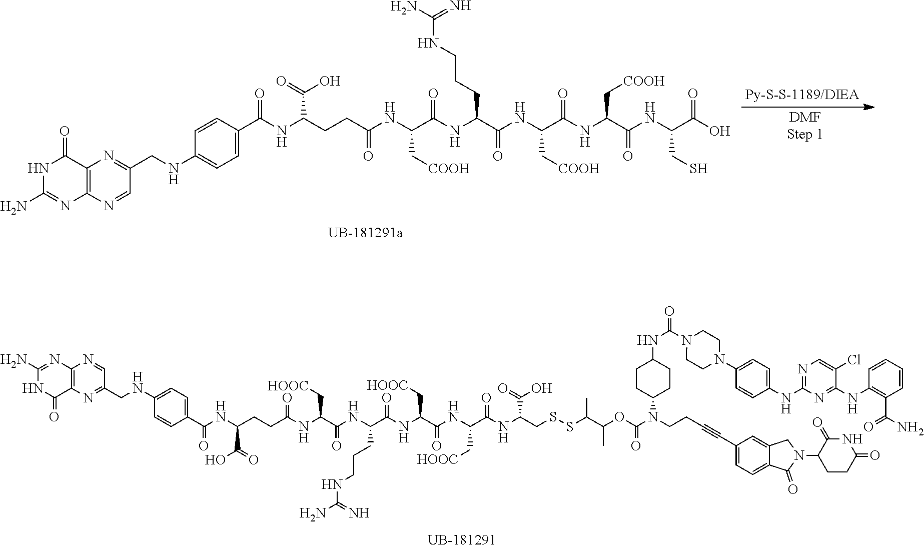

### Step 1: UB-181291

UB-181291a was prepared using solid phase synthesis (WO2011/145707 A1).

To a solution of UB-181291a (150 mg, 0.14 mmol) and Py-S—S-1189 (30 mg, 0.05 mmol) in DMF (3 ml) was added DIEA (20 mg, 0.07 mmol). The reaction solution was stirred at room temperature for 18 h and purified by preparative HPLC to obtain UB-181291 (4 mg, 1% yield) as white solid. LCMS [M+H]+=1018.9.

### Synthesis of Compound UB-181294

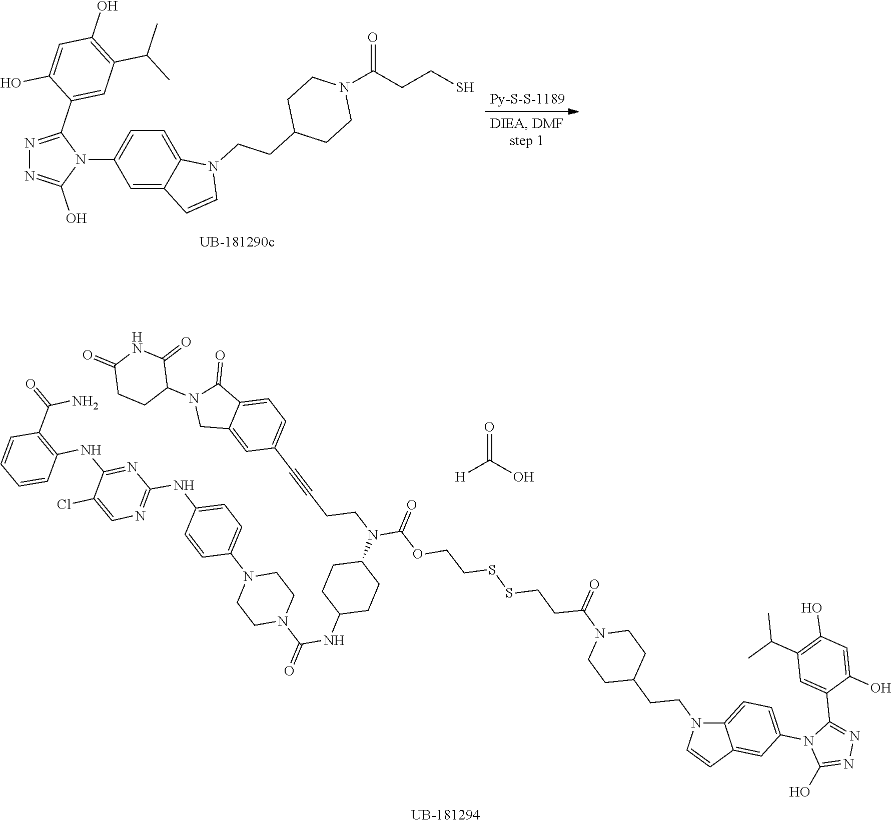

### Step 1: UB-181294

UB-181290c (50 mg, 0.09 mmol), Py-S—S-1189 (50 mg, 0.045 mmol) and DIEA (11 mg, 0.09 mmol) were dissolved in DMF (1 mL), and the mixture was reacted at room temperature for 1 hour. The reaction solution was purified by high pressure preparation (MeCN/H2O/FA) to give UB-181294 (3.8 mg, 2.8% yield) as white solid. LCMS[M/2+H]=756.90

### Synthesis of Compound UB-181295

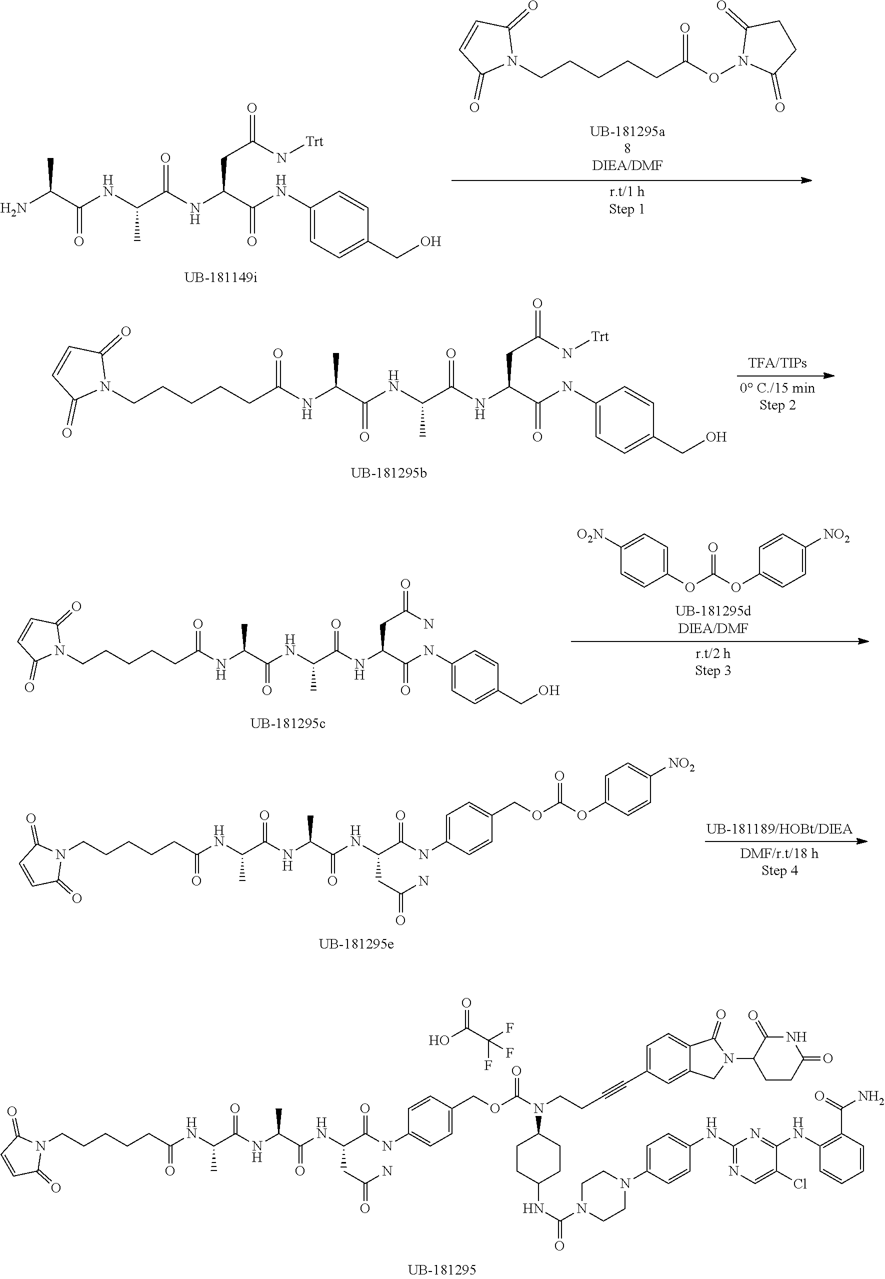

### Step 1: UB-181295b

To a solution of UB-181149i (2.66 g, 4.28 mmol) and UB-181295a (1.2 g, 3.86 mmol) in DMF (20 ml) was added DIEA (830 mg, 6.43 mmol). The reaction solution was stirred at room temperature for 18 hours. The solid obtained by pulled drying of the reaction solution via an oil pump was purified by column chromatography (DCM/DCM:MeOH:THF (10:0.5:0.5)=0-96%) to give UB-181295b (1.9 g, 60% yield) as white solid. LC-MS: [M+H]+=816.0

### Step 2: UB-181295c

To a solution of UB-181295b (500 mg, 0.6 mmol) in TIS (1 mL) was added CF3COOH (2 ml) and stirred at 0° C. for 15 min. NaHCO3 (2.25 g, 25 ml aqueous solution) was added to the reaction solution. The mixture was filtered and the filtrate was purified by reversed-phase column chromatography (H2O:acetonitrile=0%-12%) to give UB-181295c (90 mg, 25% yield) as white solid. LCMS [M+H]+=573.7

### Step 3: UB-181295e

To a solution of UUB-181295c (180 mg, 0.3 mmol) and UB-181295d (190 mg, 0.6 mmol) in DMF (15 ml) was added DIEA (81 mg, 0.6 mmol). The reaction was reacted at room temperature for 2. The reaction solution was pulled dry by an oil pump, and the resulting solid was pulped with diethyl ether. The mixture was purified by high pressure preparation (DCM/MeOH=10/1) to give UB-181295e (210 mg, 90% yield) as yellow solid. LCMS [M+H]+=738.9

### Step 4: UB-181295

To a solution of UB-181295e (210 mg, 0.28 mmol), UB-181189 (244 mg, 0.28 mmol), and HOBt (77 mg, 0.56 mmol) in DMF (2 mL) was added DIPEA (110 mg, 0.85 mmol) and stirred for 18 h at room temperature. The reaction solution was purified by preparative HPLC to obtain UB-1812957 (220 mg, 53% yield) as white solid. LCMS [M+H]+=1458.6

### Synthesis of Compound UB-181302

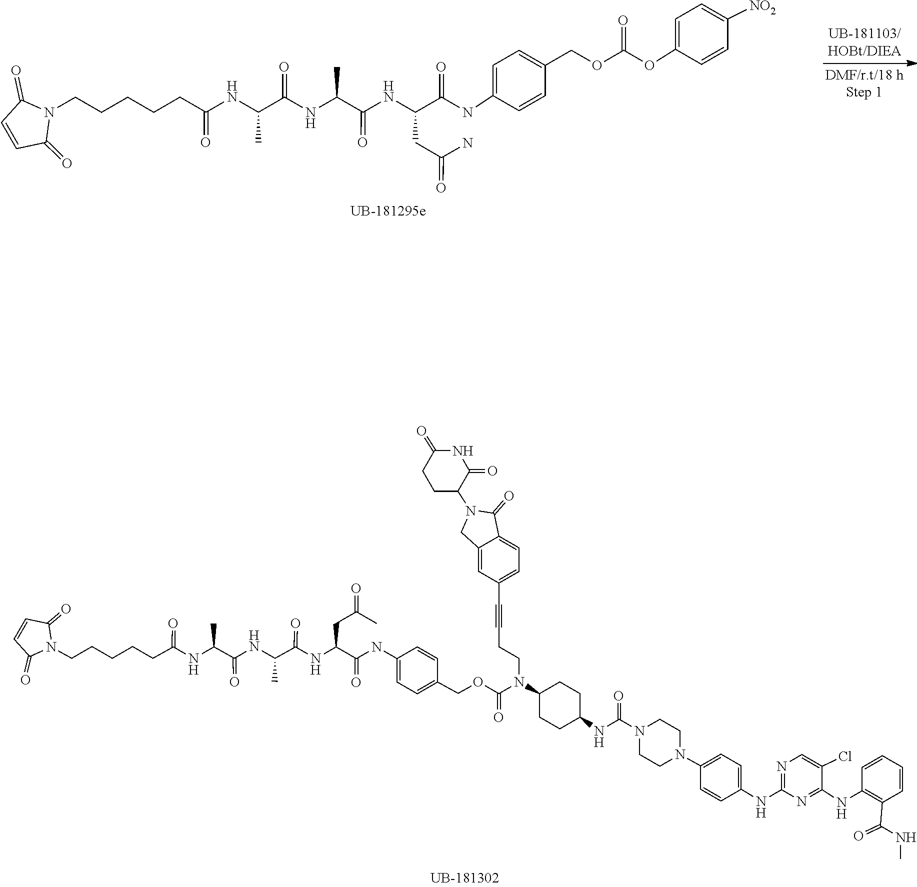

### Step 1: UB-181302

Compound UB-181295e (369 mg, 0.50 mmol), UB-181103 (349 mg, 0.40 mmol), HOBt (68 mg, 0.50 mmol) and DIPEA (194 mg, 1.50 mmol) were dissolved in DMF (5 mL), and then the mixture was reacted at room temperature overnight. The reaction solution was subjected to preparative chromatography to obtain target product UB-181302 (259 mg, yield 35%) as white solid. LCMS [M+H]⁺=1472.3

### Synthesis of Compound UB-181297

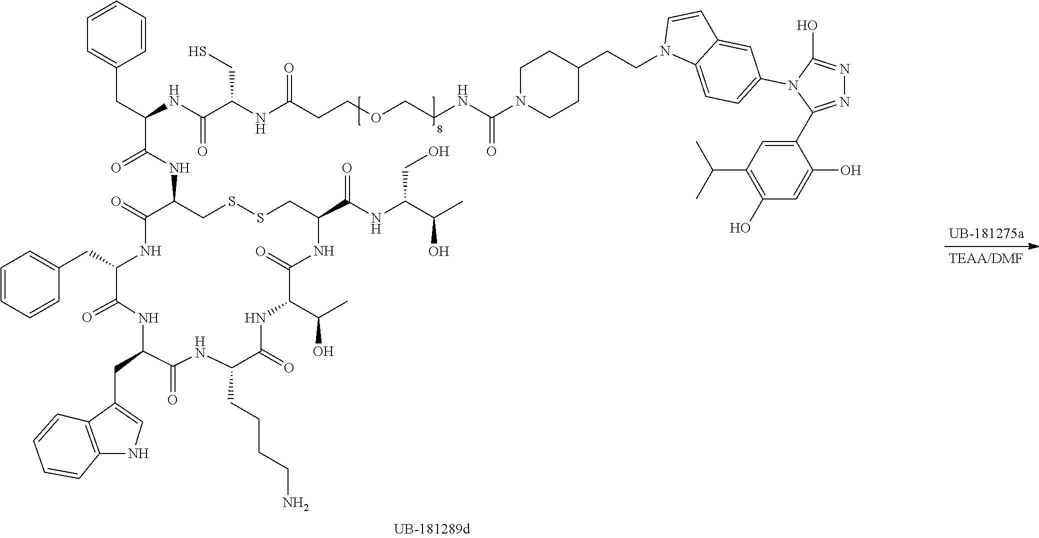

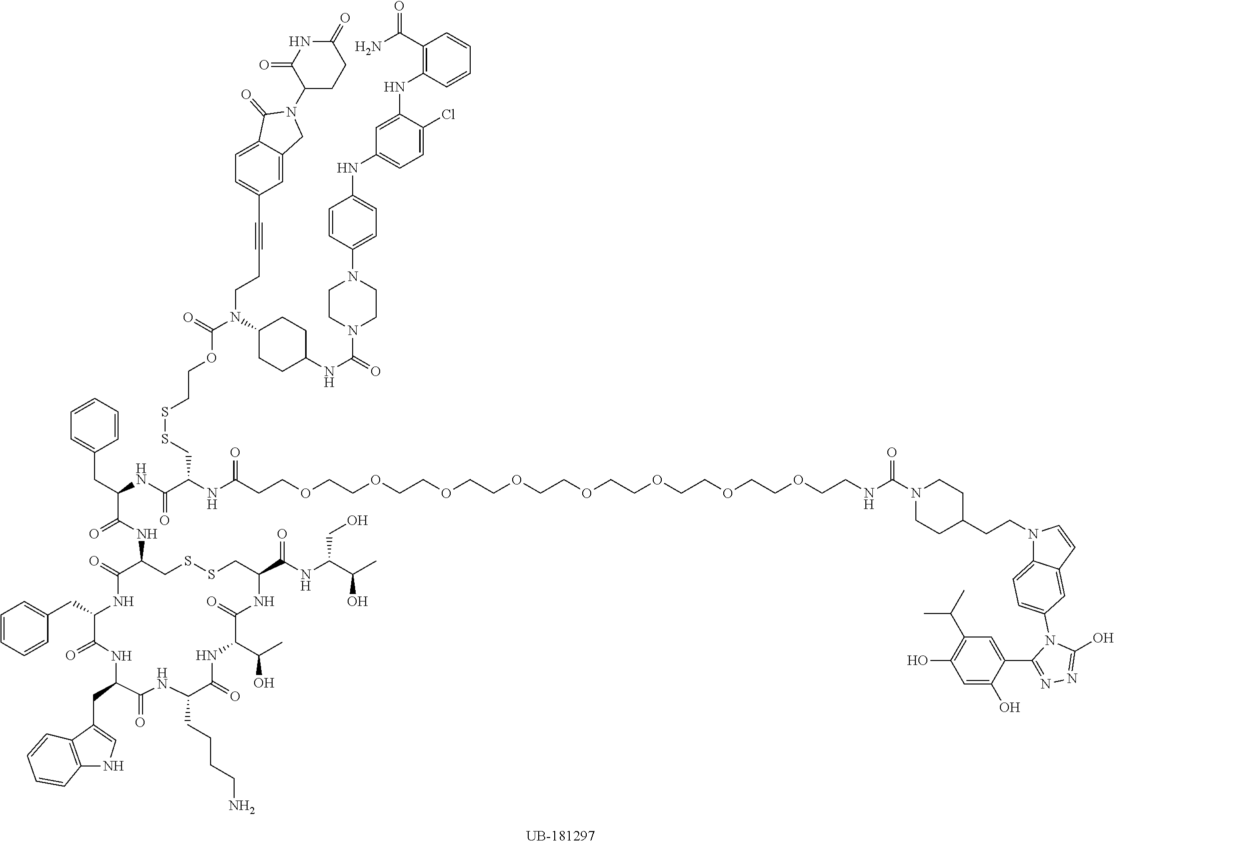

### Step 1: UB-181297

Compound UB-181285d (30 mg, 0.01 mmol) was dissolved in TEAA (2 mL), and then added with UB-181275a (16 mg, 0.01 mmol) in DMF (2.5 mL). After reacting at room temperature for 2 hours, the mixture was separated by reverse phase column chromatography to give 15 mg of crude product. The crude product was subjected to preparative chromatography to obtain target product UB-181297 (7.5 mg, 17% yield) as white solid. LCMS [M/2+H]⁺=1499.2

### Synthesis of Compound UB-181298

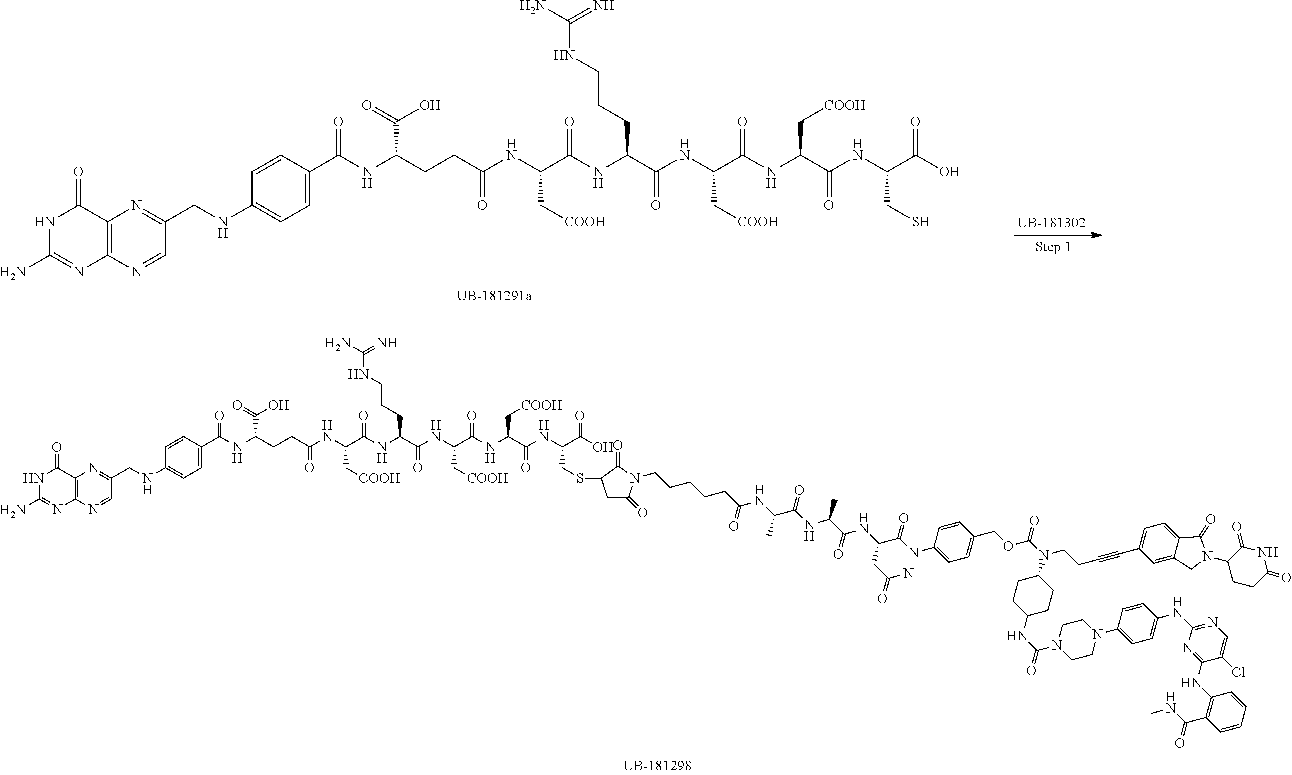

### Step 1: UB-181298

Compound UB-181291a (10 mg, 0.9*10⁻³ mmol) was dissolved in acetate buffer solution (1 mL) and added with a solution of UB-181302 (7 mg, 4.7*10-3 mmol) in DMF (2 mL). The reaction solution was left at room temperature overnight. The reaction solution was subjected to preparative chromatography to obtain product UB-181298 (9 mg, yield 76.6%) as yellow solid. LCMS [M/2+H]⁺=1260

### Synthesis of Compound UB-181299

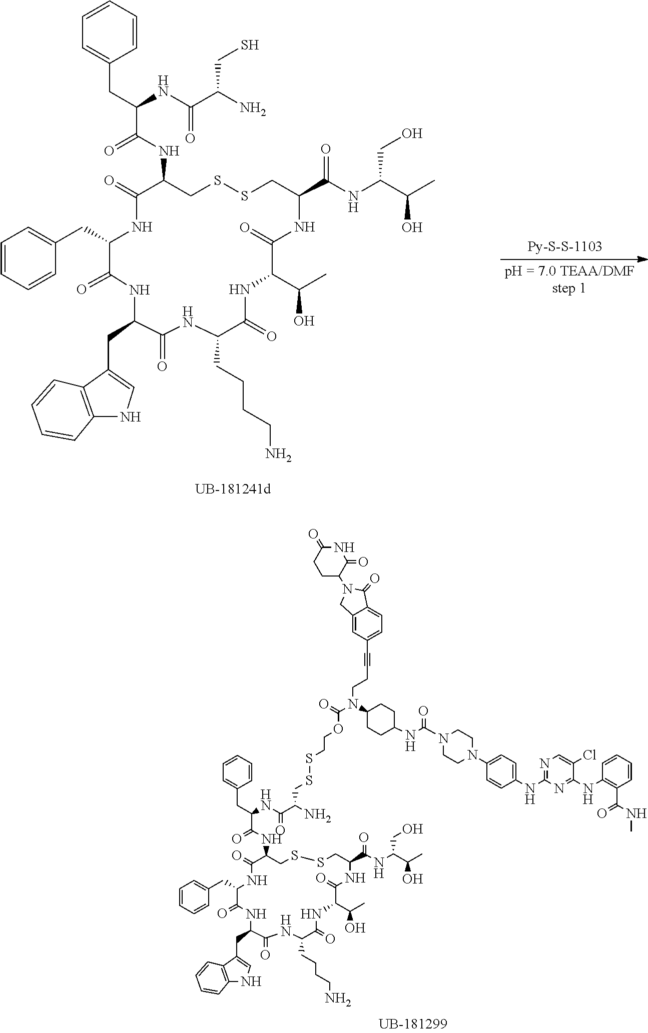

### Step 1: UB-181299

Compound UB-181241d (50 mg, 0.02 mmol) was dissolved in 1M TEAA (1 mL), and then added with Py-S—S-1103 (24 mg, 0.02 mmol) in DMF (1 mL). After reacting at room temperature for 2 hours, the mixture was separated by reversed-phase column chromatography to give 15 mg of crude product. The crude product was subjected to preparative chromatography to obtain target product UB-181299 (8 mg, yield 9%) as white solid. LCMS [M/2+H]⁺=1050.2

### Synthesis of Compound UB-181301

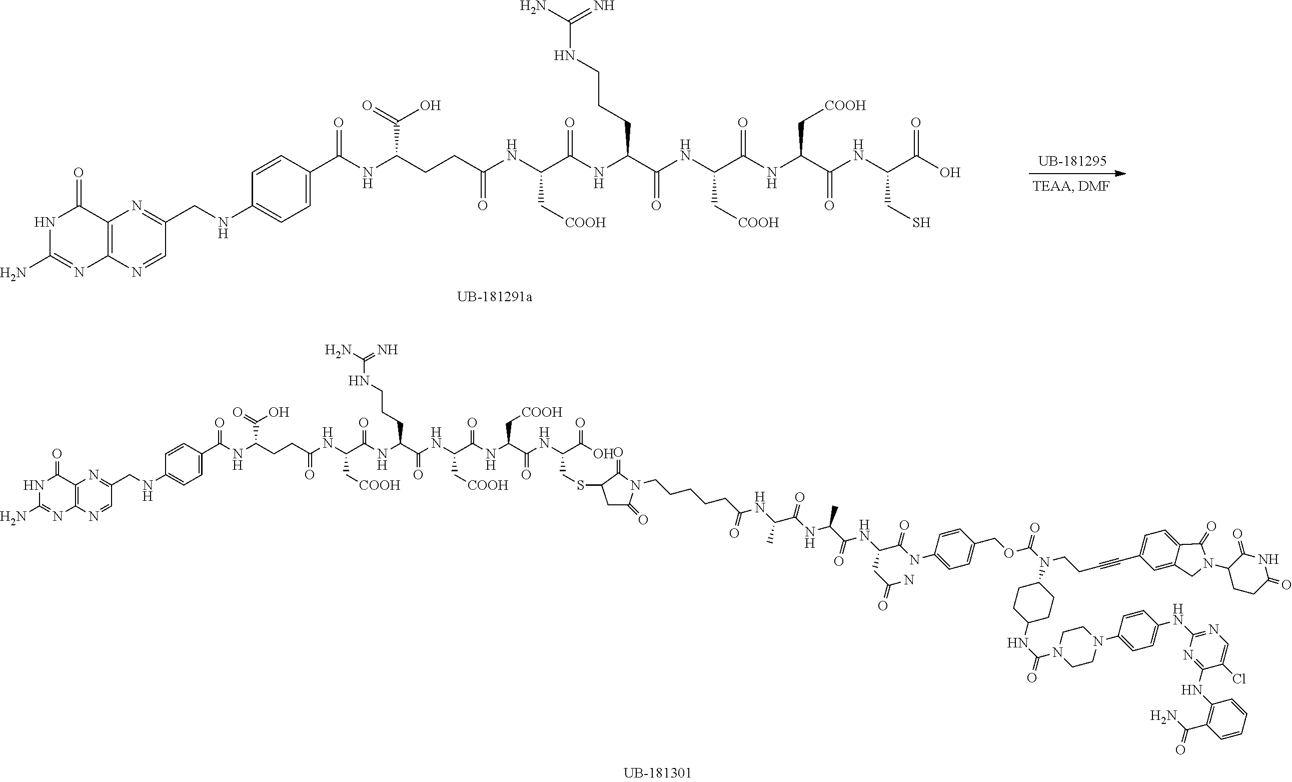

### Step 1: UB-181301

A solution of UB-181295 (93 mg, 0.064 mmol) dissolved in DMF (4 ml) was added dropwise into a reaction solution of UB-181291a (100 mg, 0.096 mmol) and TEAA (2 ml) at room temperature. After reacting at room temperature for half an hour, the reaction solution was purified by Flash (MeCN/H2O/50 mmol/l NH₄HCO₃) to obtain product UB-181301 (23.9 mg, yield 15%) as yellow solid. LCMS[M/2+H]=1253.05. ¹H NMR (400 MHz, DMSO) δ 11.84 (s, 1H), 9.72 (d, J=22.3 Hz, 1H), 9.22 (s, 1H), 8.79 (s, 1H), 8.63 (s, 1H), 8.49 (s, 2H), 8.28 (s, 3H), 8.15 (s, 3H), 7.87 (s, 1H), 7.80 (d, J=8.0 Hz, 2H), 7.75-7.55 (m, 10H), 7.52-7.38 (m, 8H), 7.31 (d, J=8.3 Hz, 3H), 7.08 (t, J=7.8 Hz, 2H), 6.90 (d, J=9.1 Hz, 5H), 6.62 (d, J=8.3 Hz, 2H), 6.11 (s, 1H), 5.08 (dd, J=14.7, 6.4 Hz, 3H), 4.61 (d, J=7.2 Hz, 2H), 4.54-4.35 (m, 6H), 4.33-4.11 (m, 7H), 4.00 (d, J=41.0 Hz, 3H), 3.78 (s, 3H), 3.47 (s, 8H), 3.03 (s, 6H), 2.68 (dd, J=7.7, 5.8 Hz, 3H), 2.57 (d, J=6.6 Hz, 5H), 2.12 (d, J=24.8 Hz, 6H), 1.97 (s, 4H), 1.83 (s, 6H), 1.64 (s, 2H), 1.47 (s, 12H), 1.21 (t, J=6.7 Hz, 9H).

### Synthesis of Compound UB-181303

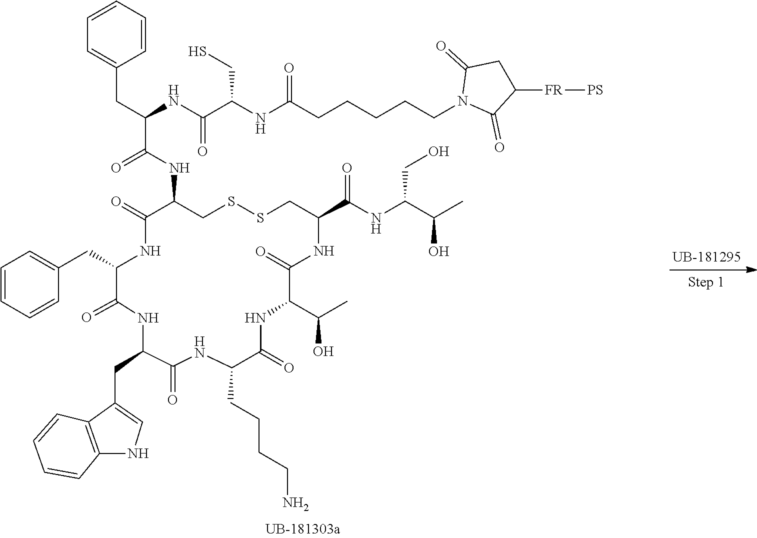

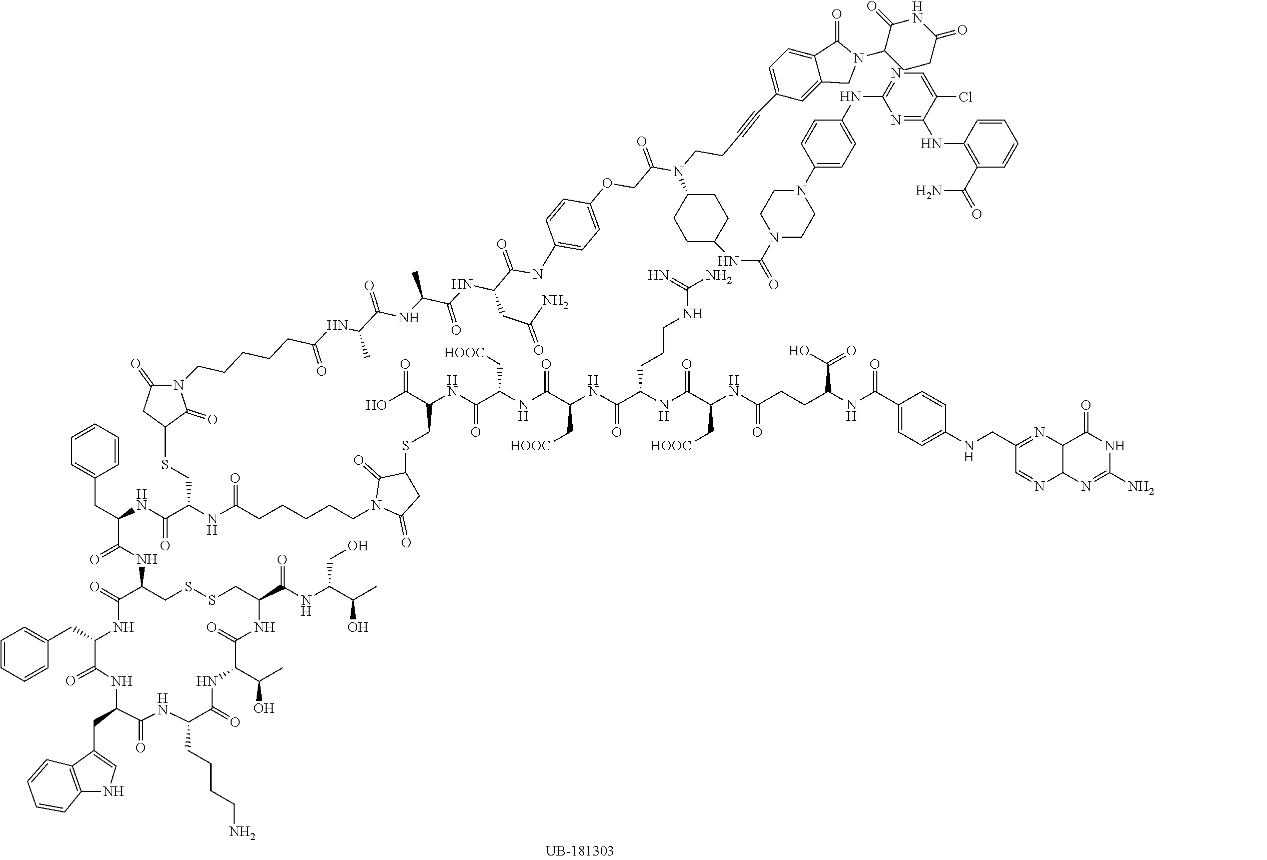

### Step 1: Step 1: UB-181303

UB-181295 (40 mg, 0.017 mmol) was dissolved in DMF (2 ml), and the solution was added dropwise to UB-181303a (40 mg, 0.016 mmol) and TEAA (1 ml) at room temperature. The mixture was reacted for half an hour at room temperature. The reaction solution was purified by C-18 reversed-phase column, MeCN/H2O/50 mmol/1 TEA, to give UB-181303 (9.5 mg, 14.5% yield) as yellow solid. LCMS[M/3+H]=1275

### Synthesis of Compound UB-181308

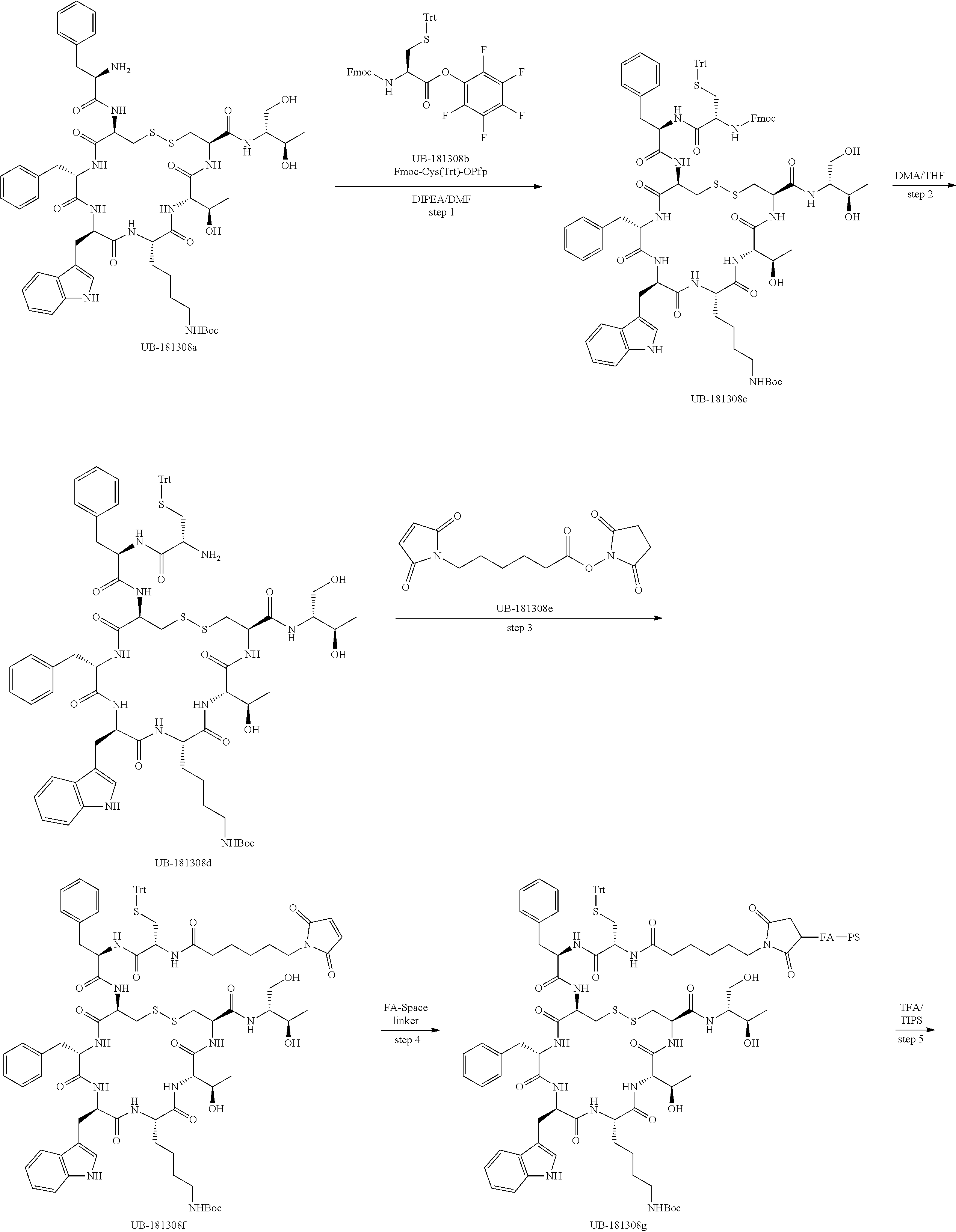

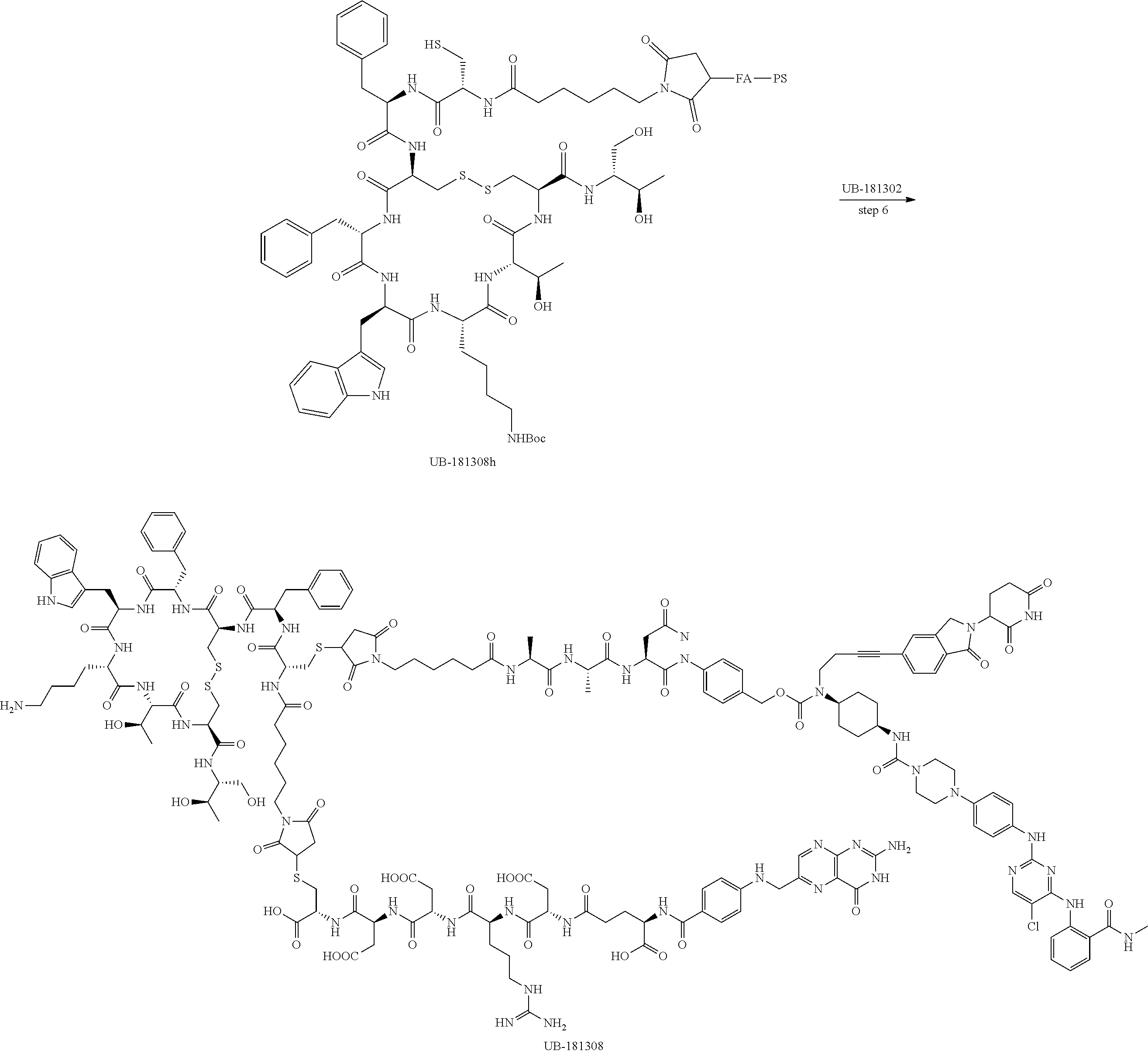

### Step 1: UB-181308c

To a solution of UB-181308a (3.1 g, 2.77 mmol) in DMF (10 mL) was added UB-181308b (1.87 g, 2.77 mmol) and DIEA (536 mg, 4.16 mmol). The reaction was stirred at room temperature for 18 h under N2 protection. The mixture was purified by reversed-phase column to give UB-181308c (3 g, 71% yield) as white solid. LCMS [M+H]=1545.7

### Step 2: UB-181308d

To a solution of UB-181308c (3 g, 1.9 mmol) in THF (50 mL) was added DMA (20 ml). The reaction solution was stirred at room temperature for 2 h. The solid obtained by spin-drying of the reaction solution was pulped with diethyl ether to obtain UB-181308d (2.0 g, 70% yield) as yellow solid. LCMS [M+H]=1465.5

### Step 3: UB-181308f

To a solution of UB-181308d (200 mg, 0.14 mmol) in DMF (5 mL) was added UB-181308e (42 mg, 0.14 mmol) and DIEA (271 mg, 0.2 mmol). The mixture was reacted at room temperature overnight. The mixture was purified by reversed-phase column (AcOH:H2O/ACN-0-100%) to give UB-181308f (30 mg, 15% yield) as white solid. LCMS [M+H]⁺=1687.5

### Step 4: UB-181308g (i.e., UB-181303a)

UB-181308f (600 mg, 0.36 mmol) was dissolved in DMF (6 ml), which was added dropwise to a solution of UB-181298a (417 mg, 0.4 mmol) and TEAA (3 ml) at room temperature. The mixture was reacted for half an hour at room temperature. The reaction solution was purified by C-18 reversed-phase column, MeCN/H2O/50 mmol/1 TEAA, to give UB-181308g (780 mg, 80% yield) as yellow solid. LCMS[M/2+H]=1353.7

### Step 5: UB-181308h

A solution of TIPS (3 ml) dissolved in TFA (30 ml) was dropped to 5 (780 mg, 0.29 mmol) at zero degrees Celsius. The mixture was reacted for half an hour at zero degrees Celsius. The reaction solution was spun dry at low temperature, washed with diisopropyl ether (100 ml), and the supernatant was poured out. This operation was repeated 4 times. The precipitate was spun dry to give product UB-181308h 7 (600 mg, 88% yield) as yellow solid. LCMS[M/2+H]=1182.1

### Step 6: UB-181308

UB-181302 (15 mg, 0.014 mmol) was dissolved in DMF (2 ml), which was added dropwise to a solution of 6 (40 mg, 0.016 mmol) and TEAA (1 ml) at room temperature. The mixture was reacted for half an hour at room temperature. The reaction solution was purified by C-18 reversed-phase column, MeCN/H2O/50 mmol/1 TEAA, to give UB-181308 (5.6 mg, 10.4% yield) as yellow solid. LCMS[M/3+H]=1279.76

### Synthesis of Compound UB-181309

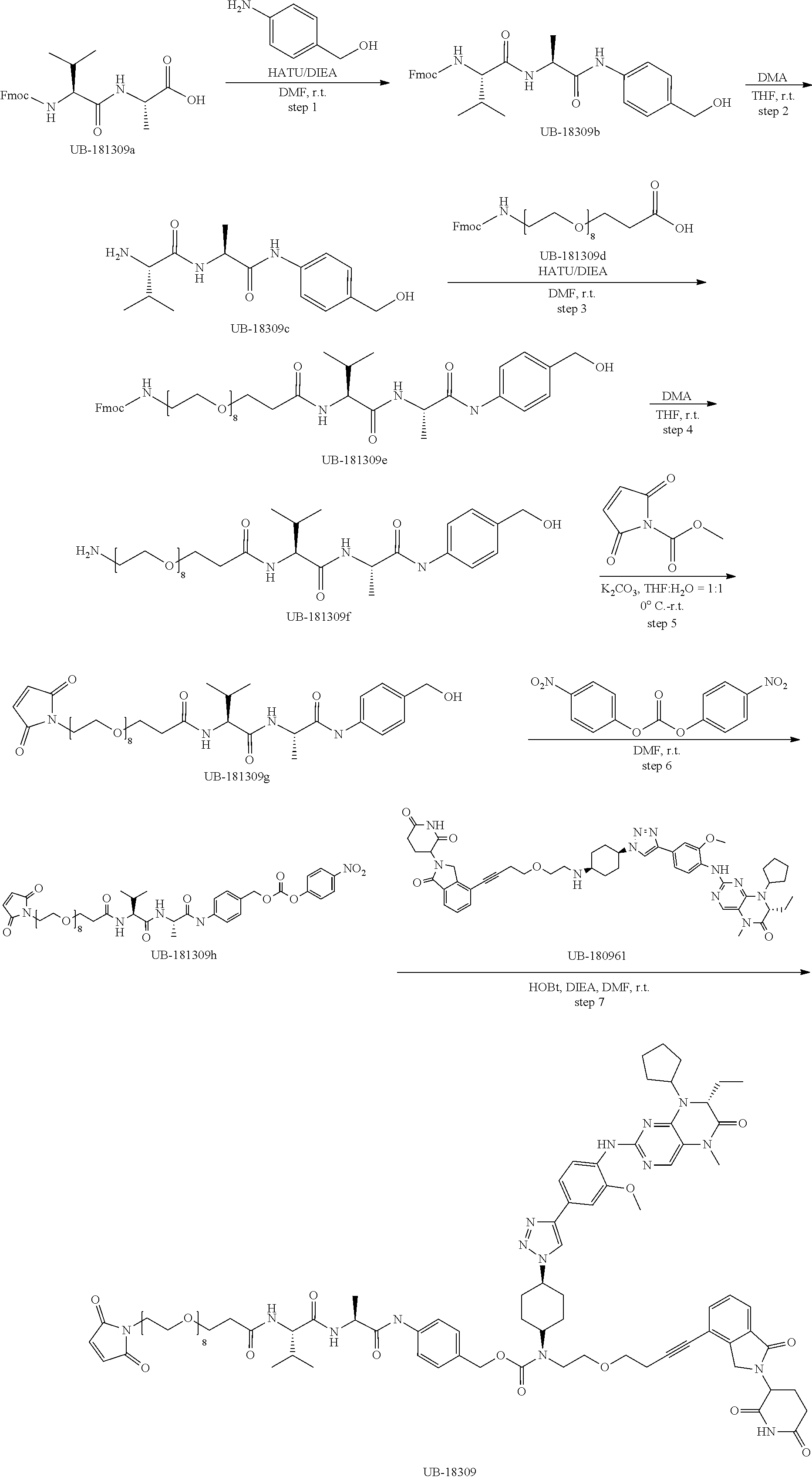

### Step 1: UB-181309b

Compound UB-181309a (5.0 g, 12.2 mmol), compound p-aminobenzyl alcohol (1.5 g, 12.2 mmol), and HATU (9.3 g, 24.4 mmol) were dissolved in DMF (50 mL). DIEA (3.2 g, 24.4 mmol) was added dropwise to the reaction solution. The mixture was then reacted at room temperature for 2 hours. Upon completion of the reaction, the reaction was concentrated under reduced pressure to remove DMF to obtain crude product, which was separated by silica gel column chromatography (dichloromethane/(methanol/tetrahydrofuran)=10%˜40% for 20 min to obtain target product as yellow solid (7.6 g crude product). LCMS: [M+1]⁺=516.

### Step 2: UB-181309c

Compound UB-181309b (3.8 g, 7.4 mmol) was dissolved in THF (40 mL). After adding DMA (25 mL, 500 mmol) to the reaction, the mixture was reacted at room temperature for 2 hours. Upon completion of the reaction, the mixture was concentrated under reduced pressure to remove THF thereby obtaining crude product. The crude product was washed with isopropyl ether (30 mL*3), and the isopropyl ether was poured. The remaining insoluble substance was concentrated under reduced pressure to obtain target product UB-181309c (2.0 g crude) as yellow oil. LCMS: [M+1]⁺=294.

### Step 3: UB-181309e

Compound UB-181309c (2 g, 6.8 mmol), UB-181309d (2.0 g, 3.0 mmol), and HATU (2.3 g, 6.0 mmol) were dissolved in DMF (20 mL). DIEA (0.77 g, 6.0 mmol) was added dropwise to the reaction solution. The mixture was then reacted at room temperature for 2 hours. After completion of the reaction, the reaction was concentrated under reduced pressure to remove DMF to obtain crude product, which was separated by silica gel column chromatography (dichloromethane/(methanol/tetrahydrofuran)=10%˜40% for 20 min to obtain the target product UBI-180857e as yellow solid (2.5 g, yield 71.4%). LCMS: [M+1]+=940.

### Step 4: UB-181309f

Compound UB-181309e (2.5 g, 2.6 mmol) was dissolved in THF (25 mL). After adding DMA (9.2 mL, 182 mmol) to the reaction, the mixture was reacted at room temperature for 2 hours. After completion of the reaction, the mixture was concentrated under reduced pressure to remove THF to obtain crude product. The crude product was washed with isopropyl ether (10 mL*3), and the isopropyl ether was poured. The remaining insoluble substance was concentrated under reduced pressure to obtain target product UB-181309f (1.9 g crude) as yellow oil. LCMS: [M+1]⁺=718.

### Step 5: UB-181309g

Compound UB-181309f (400 mg, 0.55 mmol) was dissolved in THF (4 mL), N-methoxycarbonyl maleimide (172 mg, 1.1 mmol) aqueous solution (2 mL) was added to the reaction solution, and potassium carbonate (152 mg, 1.1 mmol) aqueous solution (2 mL) was slowly added dropwise to the reaction solution at 0° C. The reaction was carried out at room temperature for 20 min. After completion of the reaction, the reaction solution was adjusted to neutral with 1N hydrochloric acid solution, and the reaction solution was directly separated by reversed-phase column (H2O/CH₃CN=20%˜60% for 20 min), and the resulting liquid was lyophilizated to obtain compound UB-181309g (140 mg, 31.4% yield) as white solid. LCMS: [M+1]⁺=798.

### Step 6: UB-181309h

Compound UB-181309g (120 mg, 0.15 mmol) and di (p-nitrophenyl)carbonate (92 mg, 0.3 mmol) were dissolved in DMF (2 mL), and added dropwise with DIEA (38.7 mg, 0.3 mmol), and the mixture was reacted at room temperature overnight. After completion of the reaction, the mixture was concentrated under reduced pressure by rotary evaporation to remove DMF thereby obtaining crude product. The crude product was washed with isopropyl ether (5 mL*3), and the isopropyl ether was poured. The remaining insoluble substance was concentrated under reduced pressure to obtain UB-181309h (152 mg crude) as fluorescent yellow oil. LCMS: [M+1]⁺=963.

### Step 7: UB-181309

Compound UB-181309h (152 mg, 0.16 mmol), UB-180961 (97.5 mg, 0.11 mol), and HOBt (42.7 mg, 0.32 mmol) were dissolved in DMF (2 mL), and added dropwise with DIEA (60.6 mg, 0.47 mmol). The mixture was reacted at room temperature overnight. The reaction solution was directly separated by reverse phase chromatography (5%0 trifluoroacetic acid aqueous solution/acetonitrile=35%˜60% for 10 minutes), and the resulting liquid was lyophilizated to obtain compound UB-181309 (21.8 mg, yield 11.5%) as white solid. [M+1]⁺=1708.

### Synthesis of Compound UB-181310

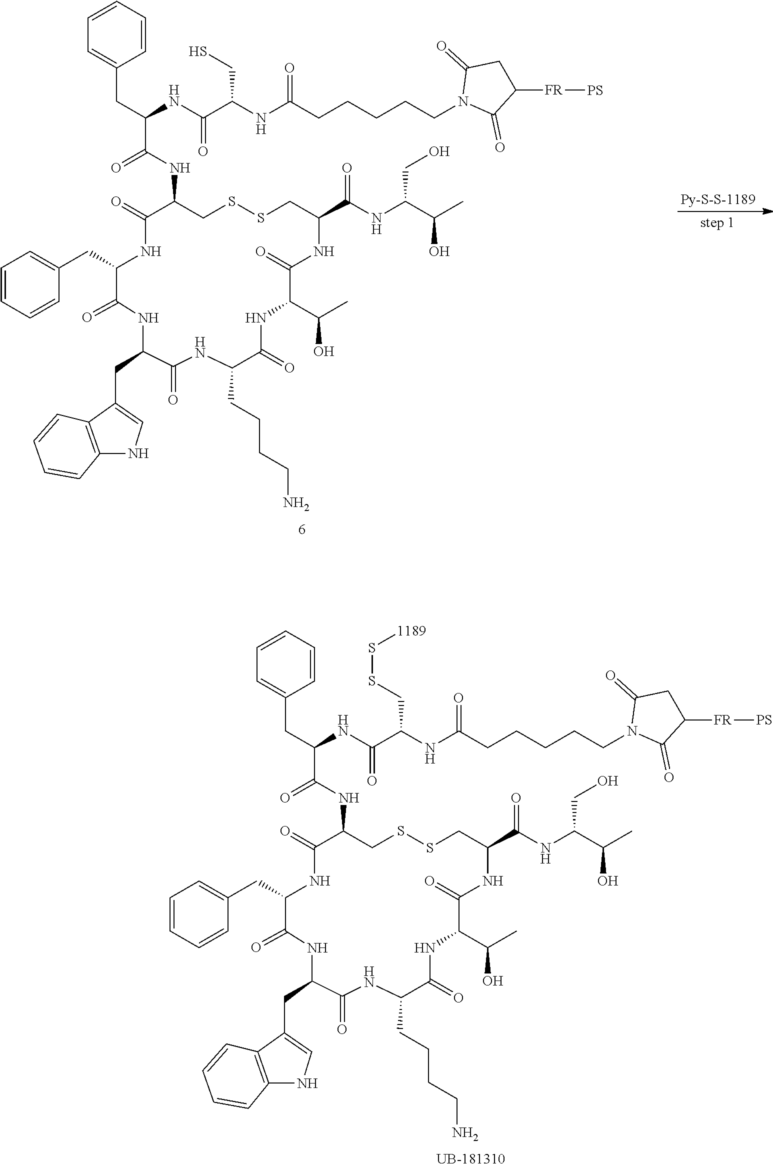

### Step 1: UB-181310

Py-S—S-1189 (15 mg, 0.014 mmol) was dissolved in DMF (2 ml), which was added dropwise to a solution of 6 (40 mg, 0.016 mmol) and TEAA (1 ml) at room temperature. The mixture was reacted for half an hour at room temperature. The reaction solution was purified by C-18 reversed-phase column, MeCN/H2O/50 mmol/l TEAA, to give yellow solid product (18 mg, 39% yield). LCMS[M/3+H]=1109.06

### Synthesis of Compound UB-181311

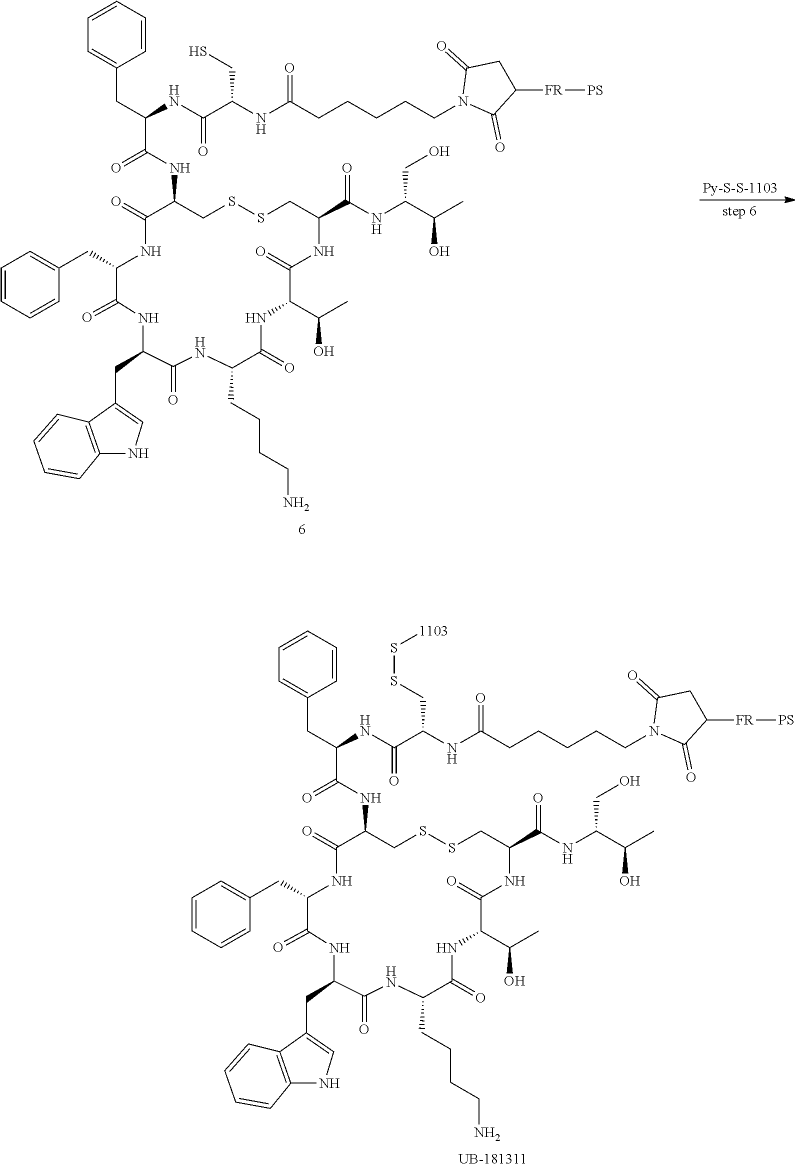

### Step 6: UB-181311

Py-S—S-1103 (15 mg, 0.014 mmol) was dissolved in DMF (2 ml), which was added dropwise to a solution of 6 (40 mg, 0.016 mmol) and TEAA (1 ml) at room temperature. The mixture was reacted for half an hour at room temperature. The reaction solution was purified by C-18 reversed-phase column, MeCN/H2O/50 mmol/l TEAA, to give UB-181311 (10.2 mg, 22% yield) as yellow solid. LCMS[M/3+H]=1122.91

### Synthesis of Compound M26

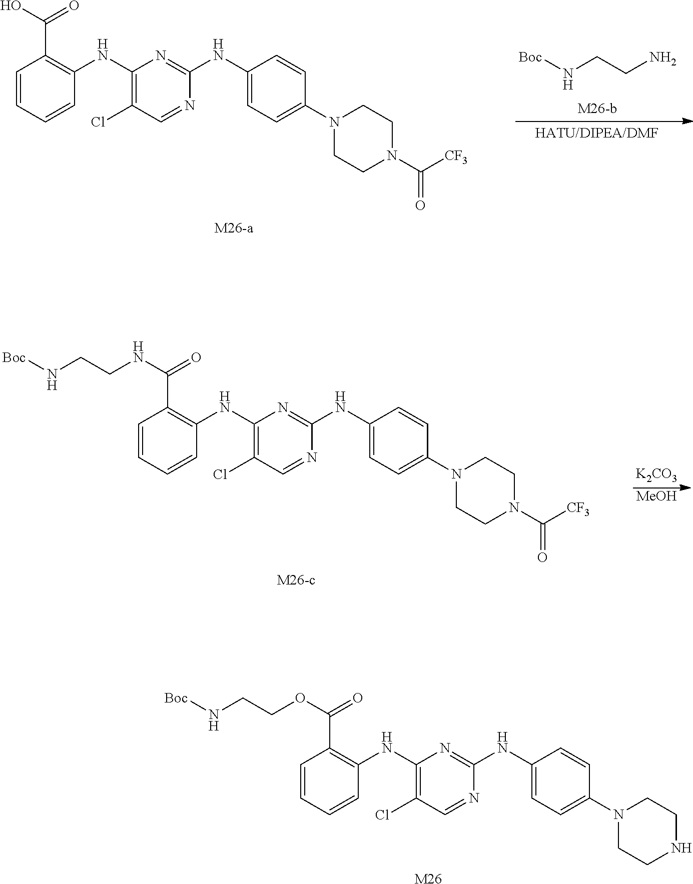

### Step 1: M26-c

Compounds M26-a (50 mg, 0.096 mmol), and M26-b (31 mg, 0.19 mmol) were dissolved in n-butanol (2 mL), followed by the addition of a catalytic amount of 4M HCl in dioxane and heated to 150° C. via microwave for 1 h. The reaction solution was concentrated to obtain M26-c as yellow solid (40 mg, yield 62.8%). LCMS [M+1]⁺=663.4

### Step 2: M26

Compound M26-c (40 mg, 0.06 mmol) was dissolved in methanol (2 mL), and then added with K₂CO₃ (42 mg). The reaction system was stirred overnight at room temperature. The reaction solution was concentrated and then separated by column chromatography (DCM:MeOH=10:1) to give M26-c (25 mg, 73% yield) as yellow solid. LCMS [M+1]⁺=568.3

### Synthesis of Compound UB-181315

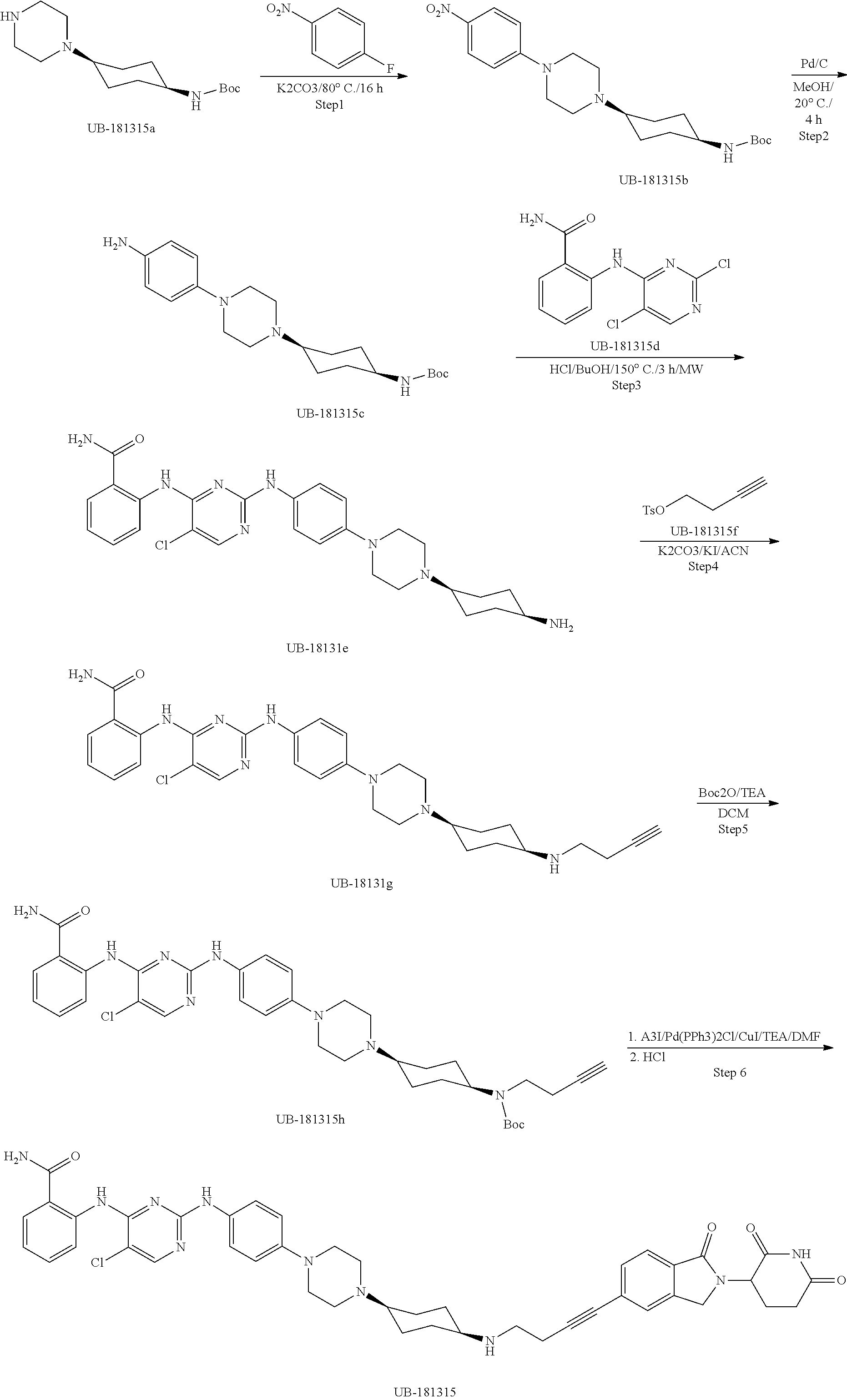

### Step 1: UB-181315b

Compound UB-181315a (700 mg, 2.47 mmol) was dissolved in ACN (20 mL), and added with p-fluoronitrobenzene (418.2 mg, 2.96 mmol), and K2CO3 (853 mg, 6.17 mmol) and reacted at 80 nC overnight. The reaction solution was filtered after cooling. The concentrated crude product was separated by column chromatography (PE/EtOAc=0-10%) to give product UB-181315b (800 mg, 80% yield) as yellow solid. LCMS [M+H]⁺=405.2

### Step 2: UB-181315c

Compound UB-181315b (800 mg) was dissolved in DCM (20 mL), and added with Pd/C (100 mg), and the mixture was reacted for 2 hours under Hz protection. The mixture was filtered, and the filtrate was concentrated to obtain crude product UB-181315c (500 mg) as yellow oil. LCMS [M+H]⁺=375.3

### Step 3: UB-181315e

Compounds UB-181315c (200 mg, 0.53 mmol), and UB-181315d (151 mg, 0.53 mmol) were dissolved in n-butanol (2 mL), followed by the addition of a catalytic amount of 4M HCl in dioxane, and the mixture was heated via microwave synthesizer to 150° C. for 1 hour. The reaction solution was concentrated and then separated by column chromatography (MeOH/DCM=1/10) to give UB-181315 (200 mg, 72% yield) as yellow solid.

### Step 4 & 5: UB-181315g

Compound UB-181315e (200 mg, 0.38 mmol) was dissolved in ACN (40 mL), and added with UB-181315f (258 g, 1.15 mmol), and K₂CO₃ (160 mg, 1.15 mmol) and the mixture was reacted at 80° C. overnight. The reaction solution was filtered after cooling. The filtrate was added with aq. NaHCO₃ (3 mL) and Boc₂O (1 mL). The mixture was reacted for 4 hours at room temperature. The reaction solution was concentrated, and the crude product was separated by column chromatography (PE/EtOAc=10˜40%) to give product UB-181315d (60 mg, 23.2% yield) as white solid. LCMS [M+H]⁺=673.4

### Step 6: UB-181315

General Method 1. LCMS [M+H]⁺=816.9

### Synthesis of Compound UB-181313

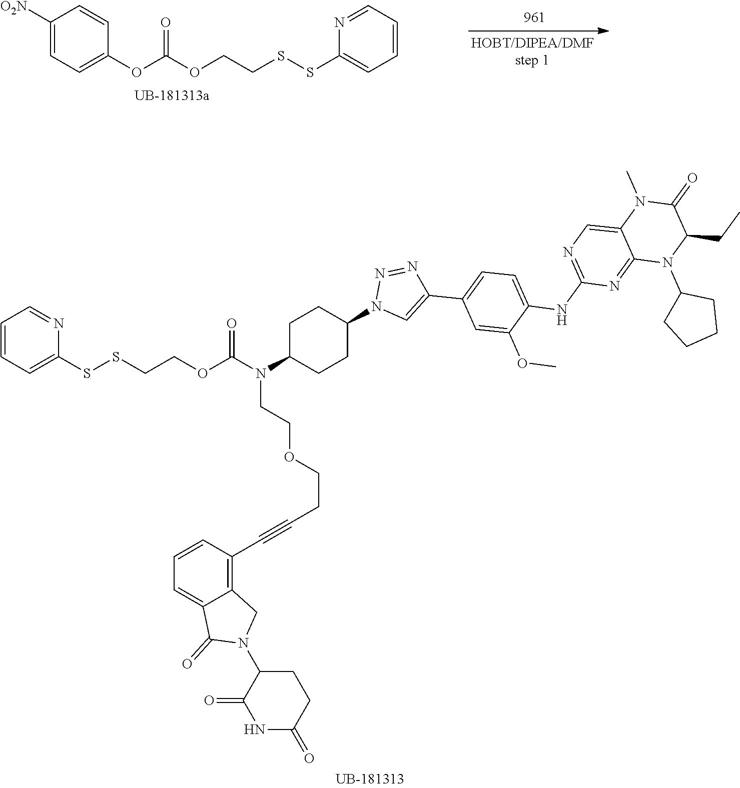 [SMILES: O=C(OCCSSc1ccccn1)Oc1ccc([N+](=O)[O-])cc1 ; CC[C@@H]1C(=O)N(C)c2cnc(Nc3ccc(-c4cn([C@H]5CC[C@@H](N(CCOCCC#Cc6cccc7c6CN(C6CCC(=O)NC6=O)C7=O)C(=O)OCCSSc6ccccn6)CC5)nn4)cc3OC)nc2N1C1CCCC1]

### Step 1: UB-181313

Compound UB-181313a (20 mg, 0.06 mmol) was dissolved in DMF (3 mL), then UB-180961 (40 mg, 0.0.05 mmol), HOBT (8 mg, 0.06 mmol) and DIEA (15 mg, 0.11 mmol) were added, and the mixture was reacted at room temperature for 16 hours. The reaction solution was subjected to preparative chromatography to obtain target product UB-181313 (35.6 mg, yield 57%) as white solid. LCMS [M+H]⁺=1098.3

### Synthesis of Compound UB-181320

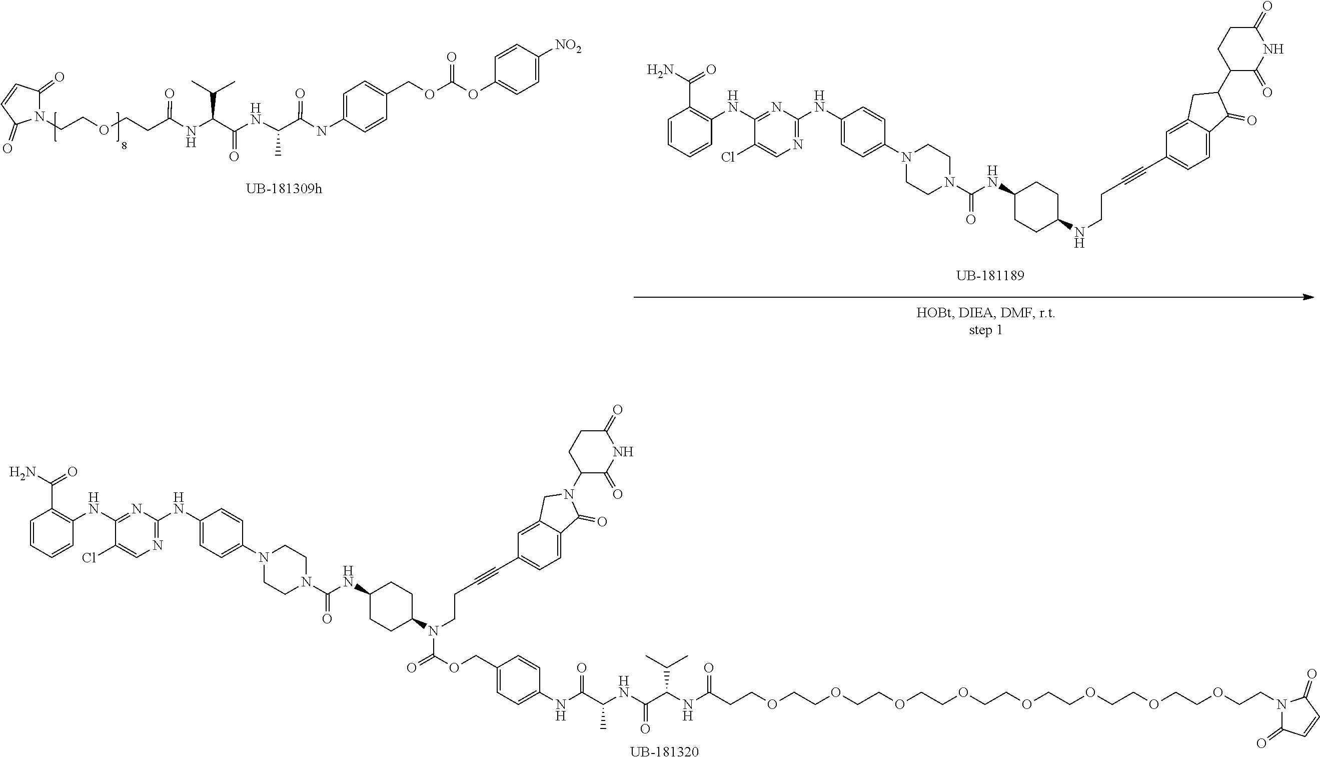

### Step 1: UB-181320

Compound UB-181309h (50 mg, 0.052 mmol), UB-181189 (44.6 mg, 0.052 mol), and HOBt (11.0 mg, 0.057 mmol) were dissolved in DMF (2 mL) followed by the dropwise addition of DIEA (1.3 mg, 0.11 mmol). The mixture was then reacted at room temperature overnight. The reaction solution was directly separated by reverse phase chromatography (5‰ trifluoroacetic acid aqueous solution/acetonitrile=35%˜60% for 10 minutes), and the resulting liquid was lyophilizated to obtain compound UB-181320 (31.3 mg, yield 32.5%) as white solid. LCMS: [M+1]⁺=1863.

### Synthesis of Compound UB-181321

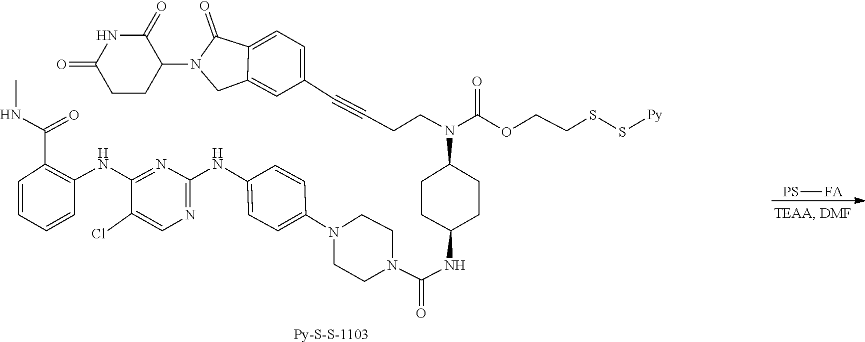

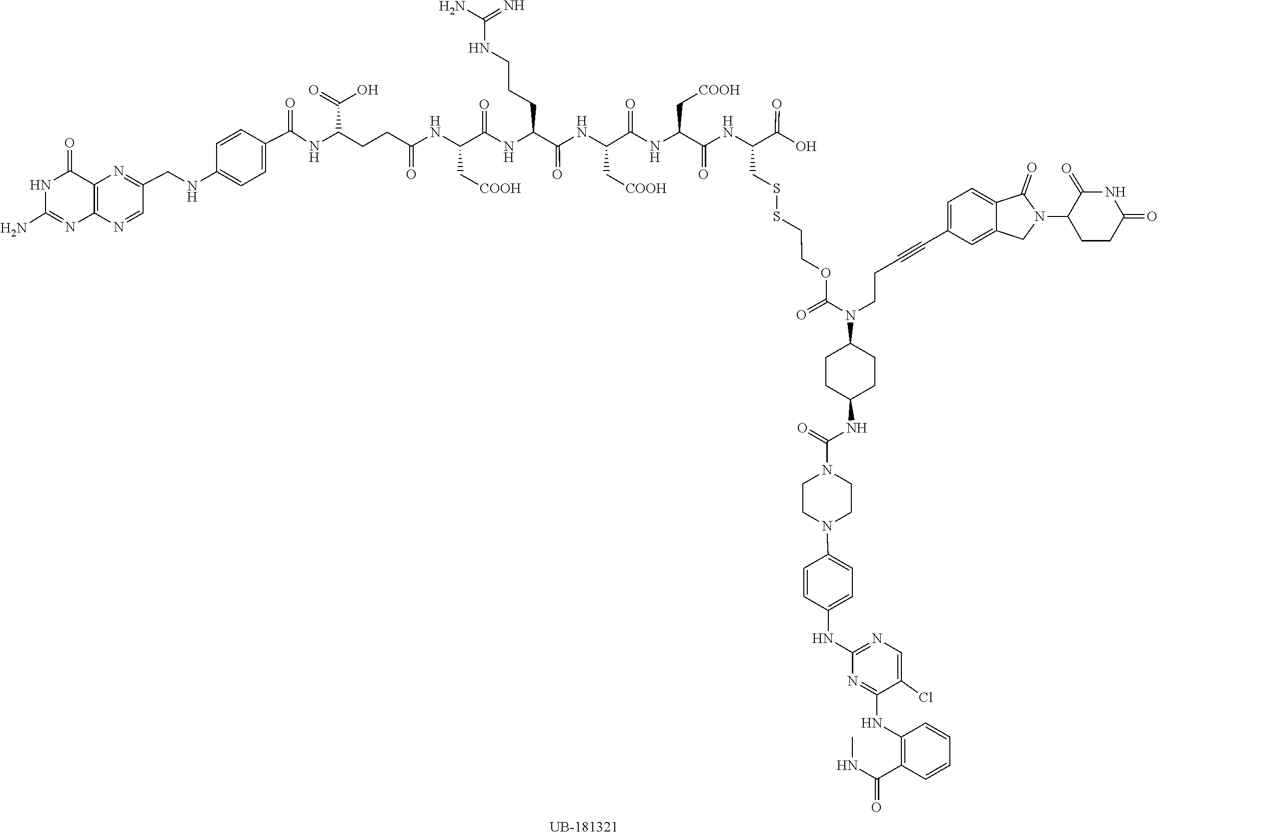

### Step 1: UB-181321

Py-S—S-1103 (35 mg, 0.033 mmol) was dissolved in DMF (2 ml), which was added dropwise to a solution of PS-FA (30 mg, 0.028 mmol) and TEAA (1 ml) at room temperature. The mixture was reacted for half an hour at room temperature. The reaction solution was purified by C-18 reversed-phase column, and medium pressure preparative chromatography, MeCN/H2O/50 mmol/1 TEAA, to give product V3441-114 (38.6 mg, 68% yield) as yellow solid. LCMS[M/2+H]=1011.33

### Synthesis of Compound UB-181322

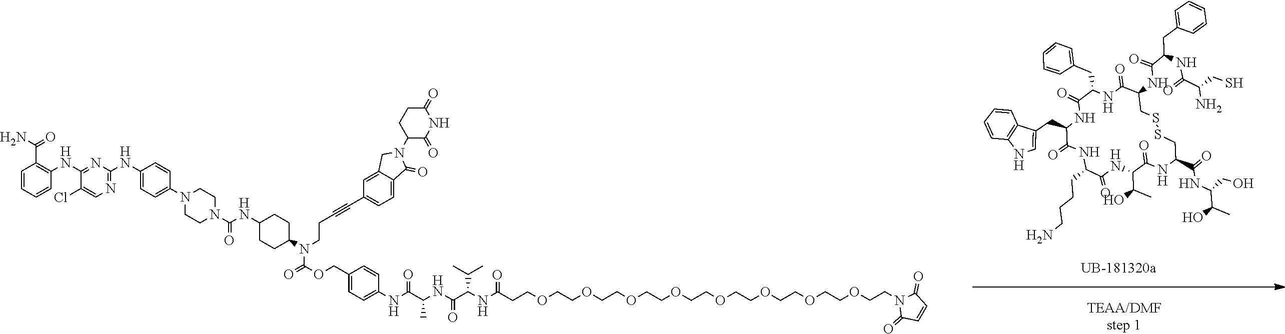

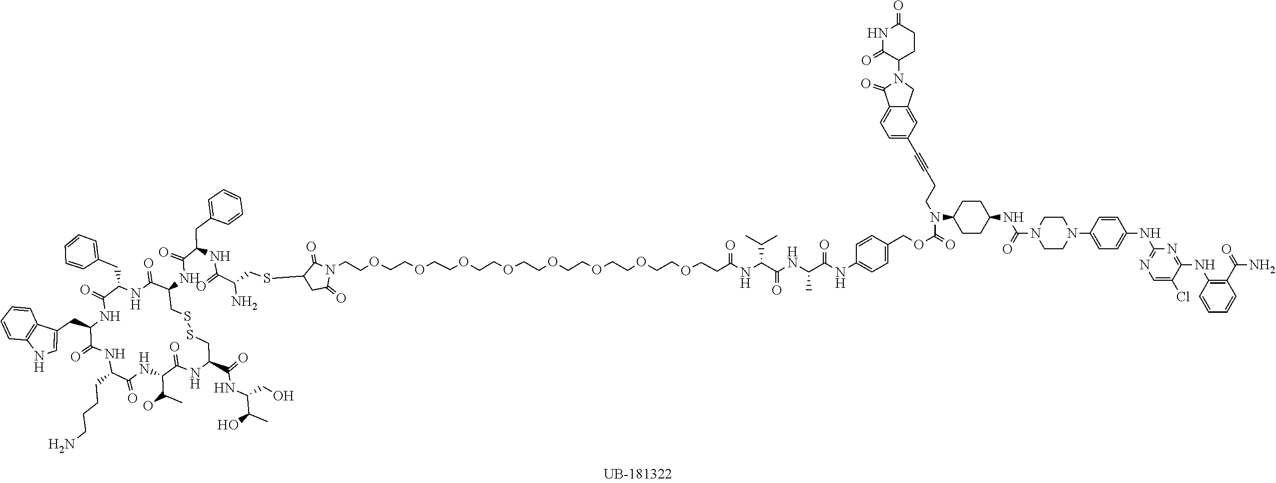

### Step 1: UB-181322

Compounds UB-181320 (30 mg, 0.016 mmol) and UB-181320a (18 mg, 0.016 mmol) were mixed and dissolved in TEEA/DMF (V/V=1:1, 3 mL). The mixture was stirred at room temperature for 1 hour and directly purified by reverse phase column (MeOH/H2O=5%˜95%, 45 min) to obtain compound UB-181322 (8.7 mg, yield 19%) as white solid. LCMS:[1/3M+1]+=935.9.

### Synthesis of Compound UB-181325

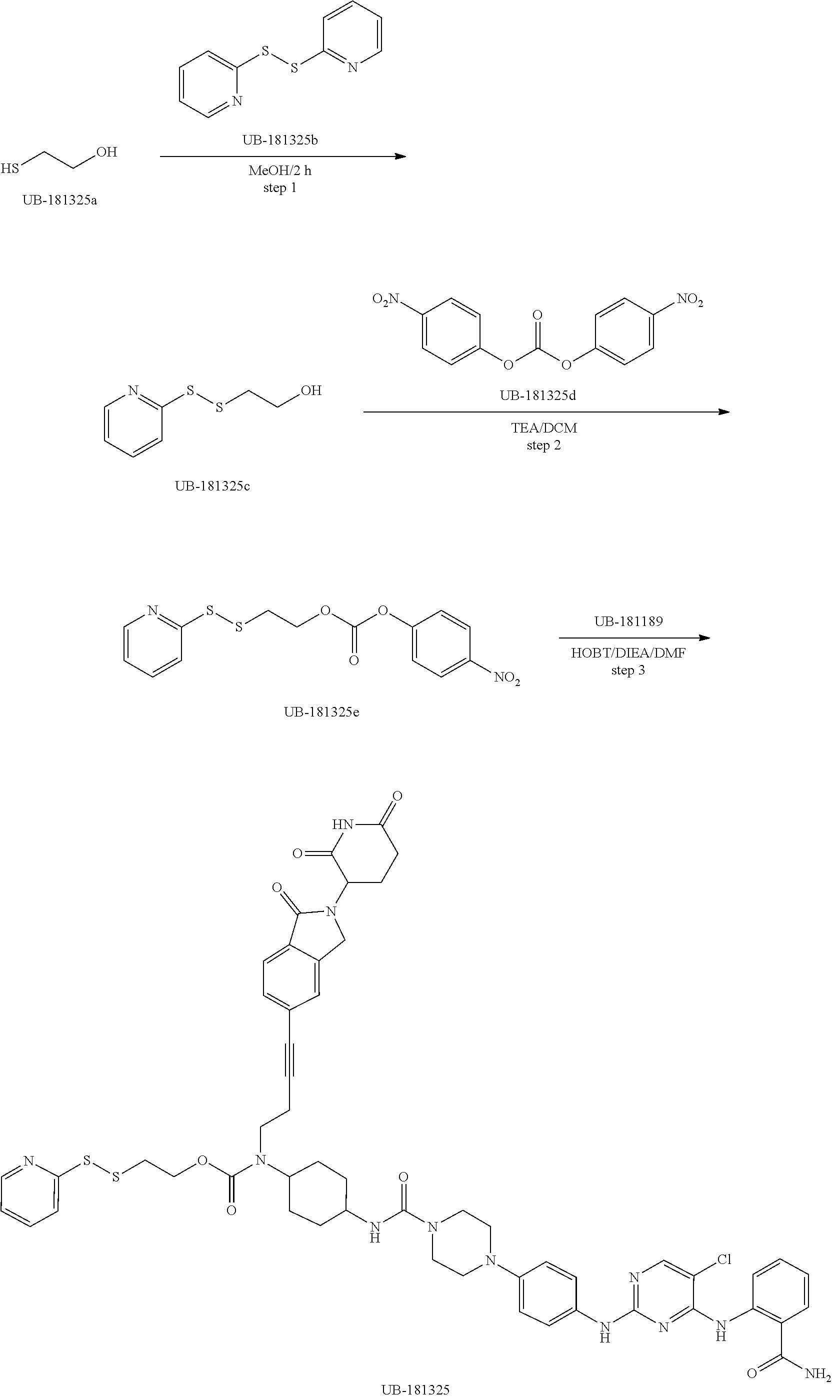

### Step 1: UB-181325c

Compound UB-181325a (500 mg, 6.4 mmol) was dissolved in MeOH (25 mL), and then UB-181325b (2.8 g, 12.8 mmol) was added. After reacting at room temperature overnight, the mixture was concentrated and separated by column chromatography (acetate ethyl/petroleum ether=1/1) to obtain target product UB-181325c (1.08g, yield 91%) as yellow oil. LCMS [M+H]⁺=188.3.

### Step 2: UB-181325e

Compound UB-181325c (1.0 g, 5.3 mmol) was dissolved in DCM (20 mL), and then UB-181325d (2.4 g, 8.0 mmol) and TEA (1.48 mL, 10.7 mmol) were added. The reaction solution was reacted at room temperature overnight, then concentrated and separated by column chromatography (dichloromethane/petroleum ether=2/1) to obtain target product UB-181325e (950 mg, yield 51%) as yellow oil. LCMS [M+H]⁺=353.5.

### Step 3: UB-181325

Compound UB-181325e (200 mg, 0.57 mmol) was dissolved in DMF (20 mL), and then UB-181189 (508 mg, 0.57 mmol), DIEA (0.2 mL, 1.14 mmol) and HOBt (77 mg, 0.57 mmol) were added. After leaving at room temperature overnight, the reaction solution was subjected to reversed-phase column chromatography to obtain target product UB-181325 (279 mg, yield 46%) as brown solid. LCMS [M+H]⁺=1072.8.

### Synthesis of Compound UB-181326

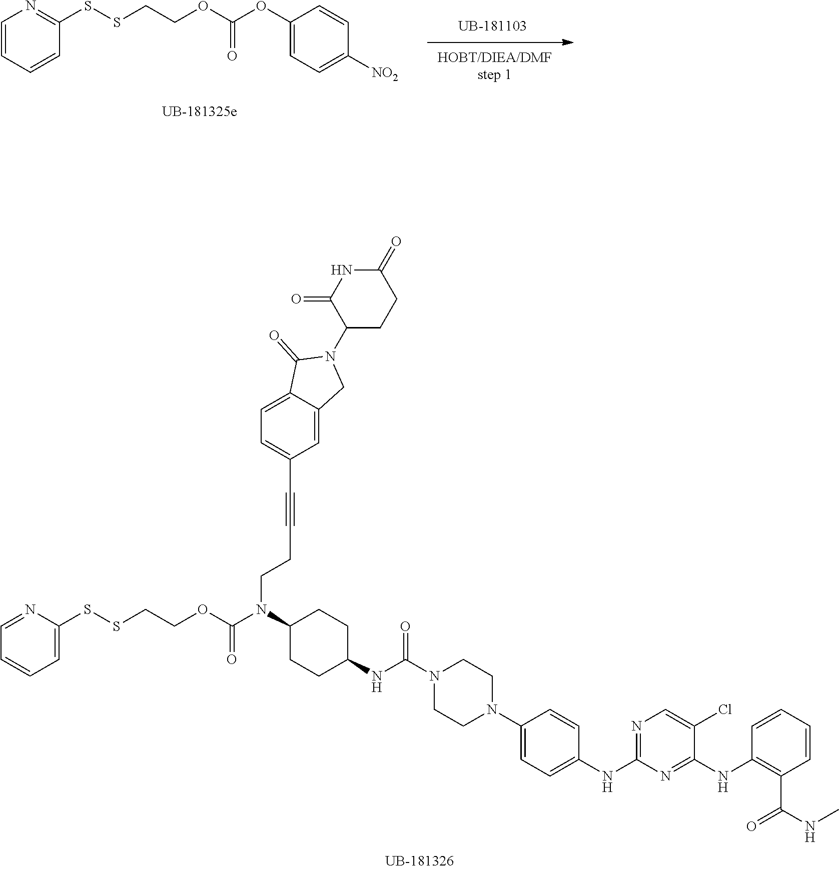 [SMILES: O=C(OCCSSc1ccccn1)Oc1ccc([N+](=O)[O-])cc1 ; CNC(=O)c1ccccc1Nc1nc(Nc2ccc(N3CCN(C(=O)N[C@H]4CC[C@@H](N(CCC#Cc5ccc6c(c5)CN(C5CCC(=O)NC5=O)C6=O)C(=O)OCCSSc5ccccn5)CC4)CC3)cc2)ncc1Cl]

### Step 1: UB-181326 (i.e., Py-S—S-1103)

Compound UB-181325e (200 mg, 0.57 mmol) was dissolved in DMF (20 mL), and then UB-181103 (495 mg, 0.57 mmol), DIEA (0.2 mL, 1.14 mmol) and HOBt (77 mg, 0.57 mmol) were added. After leaving at room temperature overnight, the reaction solution was separated by reversed phase column chromatography to obtain target product UB-181326 (270 mg, yield 44%) as brown solid. LCMS [M+H]⁺=1086.7.

Synthesis of compounds in the table below

| | | LCMS [M + H]⁺ & |
|---|---|---|
| Structure and code | Name and properties | ¹H-NMR |
| 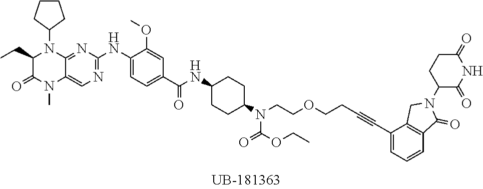 [SMILES: CCOC(=O)N(CCOCCC#Cc1cccc2c1CN(C1CCC(=O)NC1=O)C2=O)[C@H]1CC[C@@H](NC(=O)c2ccc(Nc3ncc4c(n3)N(C3CCCC3)[C@H](CC)C(=O)N4C)c(OC)c2)CC1] | 1-((1R)-1-(3-Chloro-4- (7-fluoro-1-hydroxyiso- quinolin-8-yl)phenyl)- 2-hydroxyethyl)-3-(2- ethynylthiazol-4-yl)urea White solid compound (Yield 31%) | MS[M + H]⁺ = 932.5; ¹H NMR (400 MHz, DMSO-d₆) δ 11.00 (s, 1H), 8.42 (d, J =8.4 Hz, 1H), 7.89 (d, J = 6.5 Hz, 1H), 7.82 (d, J = 1.1 Hz, 1H), 7.69 (d, J = 7.5 Hz, 1H), 7.58 (d, J = 7.7 Hz, 2H), 7.52-7.42 (m, 2H), 7.40 (d, J = 1.6 Hz, 1H), 5.13 (dd, J = 13.3, 5.1 Hz, 1H), 4.49-4.17 (m, |
| | | 4H), 4.03 (q, J = 7.0 Hz, |
| | | 3H), 3.94 (s, 3H), 3.63 |
| | | (t, J = 6.7 Hz, 2H), 3.53 |
| | | (t, J = 6.7 Hz, 2H), 3.36 |
| | | (dd, J = 13.3, 6.5 Hz, |
| | | 2H), 3.24 (s, 3H), 3.01- |
| | | 2.83 (m, 1H), 2.69 (ddd, |
| | | J = 8.5, 5.4, 4.2 Hz, 3H), |
| | | 2.57 (d, J = 18.4 Hz, |
| | | 1H), 2.47-2.37 (m, 2H), |
| | | 2.08-1.84 (m, 8H), 1.83- |
| | | 1.70 (m, 4H), 1.68-1.51 |
| | | (m, 5H), 1.46 (s, 2H), |
| | | 1.24 (d, J = 5.9 Hz, 2H), |
| | | 1.17 (t, J = 7.1 Hz, 3H), |
| | | 0.76 (t, J = 7.5 Hz, 3H). |
| 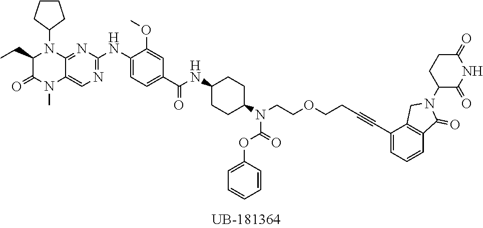 [SMILES: CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)C(=O)Oc5ccccc5)CC4)cc3OC)nc2N1C1CCCC1] | Phenyl ((1S,4s)-4-(4- (((R)-8-cyclopentyl-7- ethyl-5-methyl-6-oxo- 5,6,7,8-tetrahydro- pteridin-2-yl)amino)-3- methoxybenzamido)- cyclohexyl)(2-((4-(2- (2,6-dioxopiperidin-3- yl)-1-oxoisoindolin-4- yl)but-3-yn-1-yl)oxy)- ethyl)carbamate; white solid compound (yield 24%) | MS[M + H]⁺ = 981.4; ¹H NMR (400 MHz, DMSO-d₆) δ 10.99 (s, 1H), 8.41 (d, J = 7.8 Hz, 1H), 7.92 (s, 1H), 7.82 (d, J = 1.3 Hz, 1H), 7.68 (d, J = 7.5 Hz, 1H), 7.57 (d, J = 9.0 Hz, 2H), 7.45 (dd, J = 16.0, 8.3 Hz, 2H), 7.41-7.30 (m, 3H), 7.20 (t, J = 7.6 Hz, 1H), 7.11 (d, J = 7.5 Hz, 2H), 5.11 (dd, J = 13.2, 5.1 Hz, 1H), 4.44-4.19 (m, |
| | | 4H), 4.04 (s, 1H), 3.90 |
| | | (s, 3H), 3.67 (s, 4H), |
| | | 2.73 (t, J = 6.7 Hz, 2H), |
| | | 1.95 (dd, J = 27.0, 14.7 |
| | | Hz, 8H), 1.81-1.70 (m, |
| | | 4H), 1.69-1.48 (m, 7H), |
| | | 0.76 (t, J = 7.4 Hz, 3H). |
| 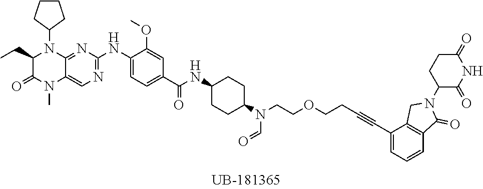 [SMILES: CC[C@@H]1C(=O)N(C)c2cnc(Nc3ccc(C(=O)N[C@H]4CC[C@@H](N(C=O)CCOCCC#Cc5cccc6c5CN(C5CCC(=O)NC5=O)C6=O)CC4)cc3OC)nc2N1C1CCCC1] | 4-((R)-8-Cyclopentyl- 7-ethyl-5-methyl-6- oxo-5,6,7,8-tetrahydro- pterin-2-yl)amino)-N- ((1s,4S)-4-(N-(2-(2,6- dioxopiperidin-3-yl)-1- oxoisooctanol-4-yl)but- 3-yn-1-yl)oxy)form- amido)cyclohexyl)-3- methoxybenzamide; white solid compound (yield 40.7%) | MS[M + H]⁺ = 888.4; ¹H NMR (400 MHz, DMSO-d₆) δ 10.99 (s, 1H), 8.21 (s, 1H), 7.99 (d, J = 32.0 Hz, 2H), 7.79 (d, J = 5.8 Hz, 1H), 7.70 (d, J = 7.5 Hz, 1H), 7.61 (t, J = 7.4 Hz, 1H), 7.54-7.40 (m, 3H), 5.14 (dd, J = 13.3, 5.1 Hz, 1H), 4.41 (t, J = 14.3 Hz, 2H), 4.33-4.16 (m, |
| | | 2H), 4.07 (s, 1H), 3.93 |
| | | (d, J = 3.2 Hz, 3H), |
| | | 3.63 (q, J = 6.5 Hz, 2H), |
| | | 3.58-3.50 (m, 2H), 3.23 |
| | | (s, 3H), 2.98-2.86 (m, |
| | | 1H), 2.71 (dd, J = 11.5, |
| | | 6.5 Hz, 2H), 2.04-1.39 |
| | | (m, 20H), 0.75 (t, J = |
| | | 7.4 Hz, 3H). |

### Synthesis of Compound UB-181363

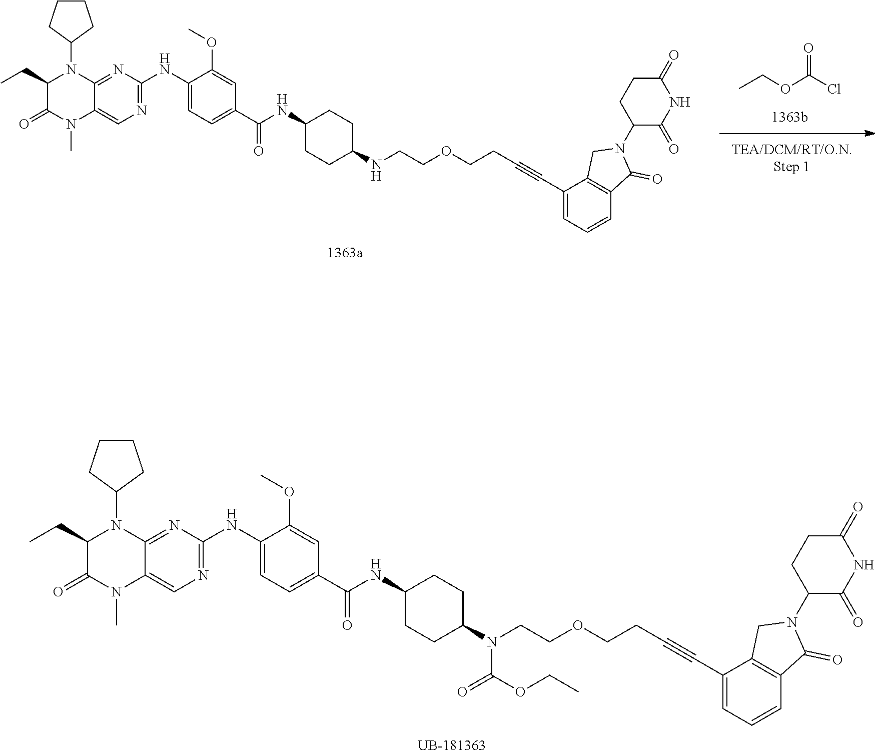

### Step 1: UB-181363

### 1-((1R)-1-(3-Chloro-4-(7-fluoro-1-hydroxyisoquinolin-8-yl)phenyl)-2-hydroxyethyl)-3-(2-ethynylthiazol-4-yl)urea

To a solution of compound 1363a (80 mg, 0.093 mmol) in DCM (5 mL) was added 1363b (12 mg, 0.111 mmol) and TEA (28 mg, 0.279 mmol) and the reaction solution was stirred at room temperature overnight. The organic phase was concentrated under reduced pressure and the residue was separated by column chromatography (eluent DCM/MeOH (10/1): DCM=0-45%) to afford compound UB-181363 (26 mg, 31% yield) as white solid. LCMS [M+H]⁺=932.5. ¹H NMR (400 MHz, DMSO-d₆) δ 11.00 (s, 1H), 8.42 (d, J=8.4 Hz, 1H), 7.89 (d, J=6.5 Hz, 1H), 7.82 (d, J=1.1 Hz, 1H), 7.69 (d, J=7.5 Hz, 1H), 7.58 (d, J=7.7 Hz, 2H), 7.52-7.42 (m, 2H), 7.40 (d, J=1.6 Hz, 1H), 5.13 (dd, J=13.3, 5.1 Hz, 1H), 4.49-4.17 (m, 4H), 4.03 (q, J=7.0 Hz, 3H), 3.94 (s, 3H), 3.63 (t, J=6.7 Hz, 2H), 3.53 (t, J=6.7 Hz, 2H), 3.36 (dd, J=13.3, 6.5 Hz, 2H), 3.24 (s, 3H), 3.01-2.83 (m, 1H), 2.69 (ddd, J=8.5, 5.4, 4.2 Hz, 3H), 2.57 (d, J=18.4 Hz, 1H), 2.47-2.37 (m, 2H), 2.08-1.84 (m, 8H), 1.83-1.70 (m, 4H), 1.68-1.51 (m, 5H), 1.46 (s, 2H), 1.24 (d, J=5.9 Hz, 2H), 1.17 (t, J=7.1 Hz, 3H), 0.76 (t, J=7.5 Hz, 3H).

### Synthesis of Compound UB-181364

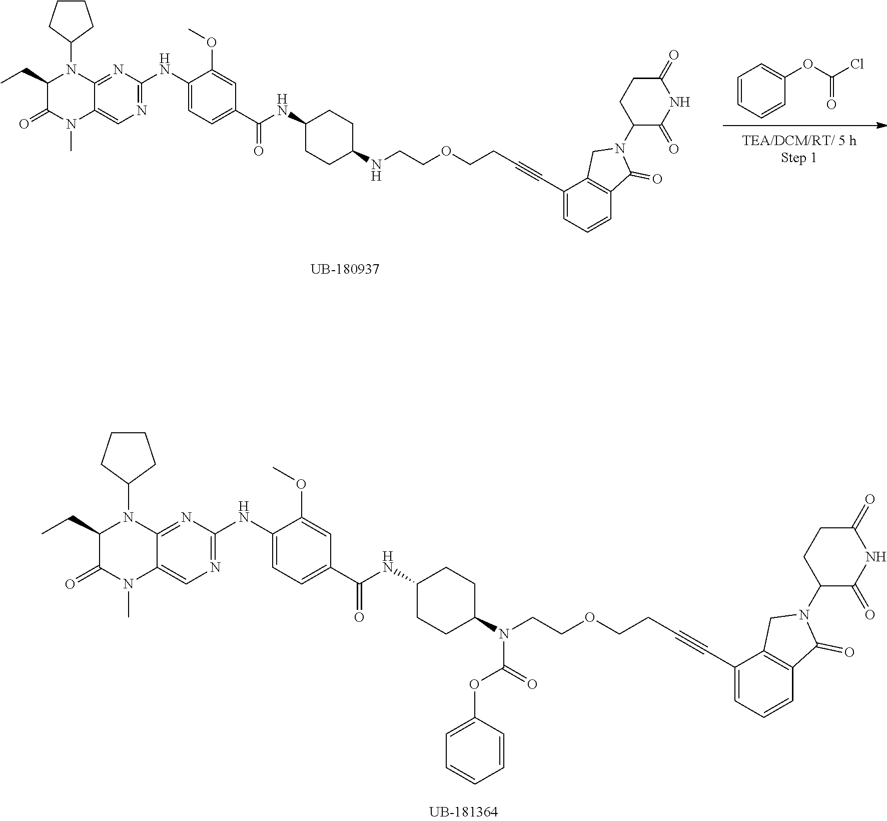 [SMILES: CC[C@@H]1C(=O)N(C)c2cnc(Nc3ccc(C(=O)N[C@H]4CC[C@@H](NCCOCCC#Cc5cccc6c5CN(C5CCC(=O)NC5=O)C6=O)CC4)cc3OC)nc2N1C1CCCC1 ; O=C(Cl)Oc1ccccc1 ; CC[C@@H]1C(=O)N(C)c2cnc(Nc3ccc(C(=O)N[C@H]4CC[C@H](N(CCOCCC#Cc5cccc6c5CN(C5CCC(=O)NC5=O)C6=O)C(=O)Oc5ccccc5)CC4)cc3OC)nc2N1C1CCCC1]

### Step 1: UB-181364

### Phenyl ((1S,4S)-4-(4-(((R)-8-cyclopentyl-7-ethyl-5-methyl-6-oxo-5,6,7,8-tetrahydropteridin-2-yl)amino)-3-methoxybenzamido)cyclohexyl) (2-((4-(2-(2,6-dioxopiperidin-3-yl)-1-oxoisoindolin-4-yl)but-3-yn-1-yl)oxy)ethyl)carbamate

UB-180937 (200 mg, 0.23 mmol), TEA (69.7 mg, 0.69 mmol), phenyl chloroformate (39.6 mg, 0.25 mmol) and DCM (5 mL) were added to a flask at room temperature. Then the solution was stirred at room temperature for 3 hours. The reaction mixture was concentrated and purified by rapid chromatography (eluent DCM/MeOH (10/1):DCM=0-35%) and then subjected to preparative chromatograph to obtain UB-181364 (55 mg, 24% yield) as white solid. LCMS [M+H]⁺=981.4. ¹H NMR (400 MHz, DMSO) δ 10.99 (s, 1H), 8.41 (d, J=7.8 Hz, 1H), 7.92 (s, 1H), 7.82 (d, J=1.3 Hz, 1H), 7.68 (d, J=7.5 Hz, 1H), 7.57 (d, J=9.0 Hz, 2H), 7.45 (dd, J=16.0, 8.3 Hz, 2H), 7.41-7.30 (m, 3H), 7.20 (t, J=7.6 Hz, 1H), 7.11 (d, J=7.5 Hz, 2H), 5.11 (dd, J=13.2, 5.1 Hz, 1H), 4.44-4.19 (m, 4H), 4.04 (s, 1H), 3.90 (s, 3H), 3.67 (s, 4H), 2.73 (t, J=6.7 Hz, 2H), 1.95 (dd, J=27.0, 14.7 Hz, 8H), 1.81-1.70 (m, 4H), 1.69-1.48 (m, 7H), 0.76 (t, J=7.4 Hz, 3H).

The synthesis of Compound UB-181365 was similar to the synthesis of UB-181363 Synthesis of compounds in the table below

| | | LCMS [M + H]⁺ & |
|---|---|---|
| Structure and code | Name and properties | ¹H-NMR |
| 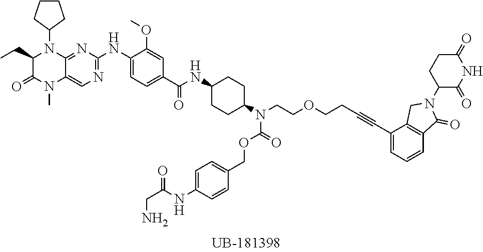 [SMILES: CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)C(=O)OCc5ccc(NC(=O)CN)cc5)CC4)cc3OC)nc2N1C1CCCC1] | 4-(2-aminoacetamido)- benzyl((1S,4s)-4-(4- ((R)-8-cyclopentyl-7- ethyl-5-methyl-6-oxo- 5,6,7,8-tetrahydro- pterin-2-yl)amino)-3- methoxybenzamido)- cyclohexyl)(2-((4-(2,6- dioxopiperidin-3-yl)-1- oxoisooctanol-4-yl)but- 3-yn-1-yl)oxy)ethyl)- carbamate; white solid compound (yield 51.0%) | MS[M + H]⁺ = 1066.4; ¹H NMR (400 MHz, DMSO-d₆)) δ 8.42 (d, J = 8.4 Hz, 1H), 7.90 (d, J = 6.1 Hz, 1H), 7.82 (d, J = 1.5 Hz, 1H), 7.68 (d, J = 7.6 Hz, 1H), 7.58 (t, J = 9.1 Hz, 4H), 7.46 (dd, J = 16.4, 8.8 Hz, 2H), 7.39 (d, J = 1.5 Hz, 1H), 7.31 (d, J = 8.4 Hz, 2H), 5.12 (dd, J = 13.3, 5.0 Hz, 1H), 5.02 (s, 2H), 4.45-4.17 (m, 4H), 4.03 (s, 1H), 3.94 (s, 3H), 3.77 (s, 2H), 3.59 |
| | | (s, 3H), 3.52 (d, J = 6.4 |
| | | Hz, 3H), 3.24 (s, 4H), |
| | | 2.73-2.63 (m, 3H), 2.06- |
| | | 1.82 (m, 12H), 1.74 |
| | | (dd, J = 13.6, 5.9 Hz, |
| | | 5H), 1.68-1.51 (m, 6H), |
| | | 1.47 (s, 2H), 1.07-0.82 |
| | | (m, 2H), 0.76 (t, J = 7.5 |
| | | Hz, 3H). |
| 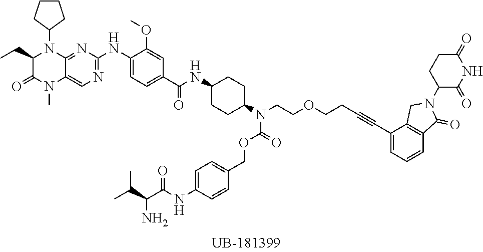 [SMILES: CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)C(=O)OCc5ccc(NC(=O)[C@@H](N)C(C)C)cc5)CC4)cc3OC)nc2N1C1CCCC1] | 4-((S)-2-amino-3-meth- ylbutylamino)benzyl- ((1S,4R)-4-(4-(((R)-8- cyclopentyl-7-ethyl-5- methyl-6-oxo-5,6,7,8- tetrahydropterin-2-yl)- amino)-3-methoxy- benzamido)cyclohexyl)- (2-(4-(2-(6-dioxopiper- idin-3-yl)-1-oxoisoin- dolin-4-yl)but-3-yn-1- yl)oxy)ethyl)carbamate White solid compound (yield 40.9%) | MS[M + H]⁺ = 1108.3; ¹H NMR (400 MHz, DMSO-d₆) δ 8.42 (d, J = 8.4 Hz, 1H), 7.90 (d, J = 5.4 Hz, 1H), 7.82 (d, J = 1.3 Hz, 1H), 7.68 (d, J = 7.5 Hz, 1H), 7.59 (dd, J = 15.8, 9.0 Hz, 4H), 7.46 (dd, J = 16.1, 8.5 Hz, 2H), 7.40 (s, 1H), 7.29 (d, J = 8.4 Hz, 2H), 5.12 (dd, J = 13.4, 4.9 Hz, 1H), 5.01 (s, 2H), 4.46-4.18 (m, 5H), 4.03 (s, 1H), 3.93 (s, 3H), 3.77 (s, 1H), |
| | | 3.65-3.49 (m, 5H), 3.24 |
| | | (s, 3H), 3.10 (d, J = 4.9 |
| | | Hz, 1H), 2.98-2.84 (m, |
| | | 1H), 2.67 (d, J = 1.8 |
| | | Hz, 3H), 2.06-1.70 (m, |
| | | 17H), 1.68-1.40 (m, |
| | | 8H), 0.91 (d, J = 6.8 |
| | | Hz, 3H), 0.83 (d, J = |
| | | 6.7 Hz, 3H), 0.76 (t, |
| | | J = 7.4 Hz, 3H). |
| 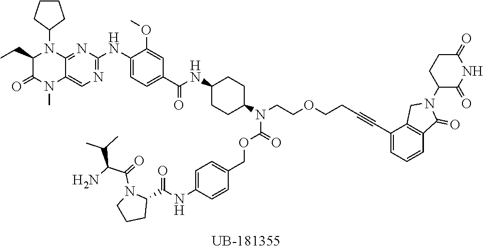 [SMILES: CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)C(=O)OCc5ccc(NC(=O)[C@@H]6CCCN6C(=O)[C@@H](N)C(C)C)cc5)CC4)cc3OC)nc2N1C1CCCC1] | ethyl 4-((S)-1-(L-pro- pionyl)pyrrolidin-2- formamido)benzyl ((1S,4R)-4-(4-(((R)-8- cyclopentyl-7-ethyl-5- methyl-6-oxo-5,6,7,8- tetrahydropterin-2-yl)- amino)-3-methoxybenz- amido)(2-((4-(2-(2,6- dioxopiperidin-3-yl)-1- oxoisoindolin-4-yl)but- 3-yn-1-yl)oxy)carba- mate White solid com- pound (yield 51.6%) | MS[M/2 + 1]⁺ = 603.2; ¹H NMR (400 MHz, DMSO-d₆) δ 10.07(s, 1H), 8.42 (d, J = 8.4 Hz, 1H), 7.90 (d, J = 5.7 Hz, 1H), 7.82 (d, J = 1.6 Hz, 1H), 7.68 (d, J = 7.5 Hz, 1H), 7.56 (dd, J = 9.1, 4.8 Hz, 4H), 7.50-7.38 (m, 3H), 7.31 (t, J = 9.5 Hz, 2H), 5.12 (dd, J = 13.3, 5.1 Hz, 1H), 5.01 (s, 2H), 4.47 (dd, J = 8.1, 5.4 Hz, 1H), 4.42- 4.18 (m, 4H), 4.02 (s, 1H), 3.93 (s, 3H), 3.81- |
| | | 3.68 (m, 2H), 3.64-3.47 |
| | | (m, 7H), 3.24 (s, 3H), |
| | | 2.97-2.84 (m, 1H), 2.67 |
| | | (dd, J = 3.7, 1.8 Hz, |
| | | 2H), 2.17 (d, J = 6.1 Hz, |
| | | 1H), 2.06-1.69 (m, |
| | | 19H), 1.68-1.38 (m, |
| | | 7H), 0.96 (d, J = 6.7 Hz, |
| | | 3H), 0.88 (d, J = 6.6 Hz, |
| | | 3H), 0.76 (t, J = 7.4 Hz, |
| | | 3H). |
| 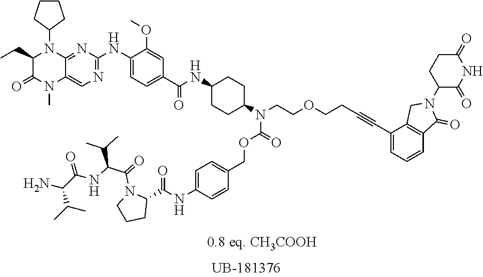 [SMILES: CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)C(=O)OCc5ccc(NC(=O)[C@@H]6CCCN6C(=O)[C@@H](NC(=O)[C@@H](N)C(C)C)C(C)C)cc5)CC4)cc3OC)nc2N1C1CCCC1] | 4-(S)-1-((S)-2-((S)-2- amino-3-methylbutyl- amido)-3-methylbut- yryl)pyrrolidin-2-form- amido)benzyl((1S,4R)- 4-(4-(((R)-8-cyclopent- yl-7-ethyl-5-methyl-6- oxo-5,6,7,8-tetrahydro- pterin-2-yl)amino)-3- methoxybenzamido)(2- (4-(2-(2,6-dioxopiper- idin-3-yl)-1-oxoiso- octanol-4-yl)but-3-yn- 1-yl)oxy)ethyl)carba- mate; white solid com- pound (yield 38%) | MS[M/2 + 1]⁺ = 653.0; ¹H NMR (400 MHz, DMSO-d₆) δ 10.98 (s, 1H), 10.06 (s, 1H), 8.42 (d, J = 8.4 Hz, 1H), 8.19 (s, 1H), 7.90 (s, 1H), 7.82 (d, J = 1.5 Hz, 1H), 7.68 (d, J = 7.5 Hz, 1H), 7.60-7.54 (m, 4H), 7.46 (dd, J = 16.7, 8.8 Hz, 2H), 7.39 (s, 1H), 7.29 (d, J = 8.2 Hz, 2H), 5.12 (dd, J = 13.2, 5.0 Hz, 1H), 5.01 (s, 2H), 444- 428 (m, 5H), 4.22 (dd, J = 7.7, 3.5 Hz, 1H), 4.02 (s, 1H), 3.93 (s, |
| | | 3H), 3.81 (s, 1H), 3.76 |
| | | (s, 1H), 3.59 (s, 3H), |
| | | 3.52 (s, 2H), 3.39 (s, |
| | | 3H), 3.24 (s, 4H), 2.91 |
| | | (dd, J = 22.0, 9.3 Hz, |
| | | 1H), 2.67 (d, J = 1.8 Hz, |
| | | 3H), 2.58 (s, 1H), 2.43- |
| | | 2.37 (m, 2H), 2.33 (s, |
| | | 2H), 2.13 (s, 2H), 2.05- |
| | | 1.70 (m, 22H), 1.69- |
| | | 1.51 (m, 6H), 1.47 (s, |
| | | 2H), 0.94 (d, J = 6.6 Hz, |
| | | 3H), 0.87 (d, J = 6.8 Hz, |
| | | 6H), 0.80 (d, J = 6.8 Hz, |
| | | 3H), 0.75 (t, J = 7.4 Hz, |
| | | 3H). |
| 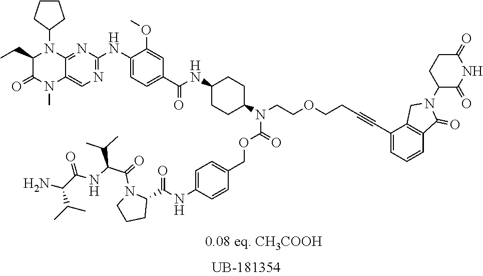 | 4-((S)-1-(L-propionyl- L-prolyl-L-propionyl)- pyrrolidin-2-form- amido)benzyl((1S,4R)- 4-(4-(((R)-8-cyclopent- yl-7-ethyl-5-methyl-6- oxo-5,6,7,8-tetrahydro- pterin-2-yl)amino)-3- methoxybenzamido)(2- ((4-(2-(2,6-dioxopiper- idin-3-yl)-1-oxoiso- octanol-4-yl)but-3-yn- 1-yl)oxy)ethyl)carba- mate; white solid com- pound (yield 65.1%) | MS[M/2 + 1]⁺ = 701.5; ¹H NMR (400 MHz, DMSO-d₆) δδ 11.31- 10.65 (m, 1H), 10.04 (d, J = 15.3 Hz, 1H), 8.42 (d, J = 8.4 Hz, 1H), 7.90 (s, 1H), 7.85 (d, J = 8.3 Hz, 1H), 7.82 (d, J = 1.5 Hz, 1H), 7.68 (d, J = 7.5 Hz, 1H), 7.60-7.54 (m, 4H), 7.46 (dd, J = 16.8, 8.8 Hz, 2H), 7.39 (s, 1H), 7.29 (d, J = 8.5 Hz, 2H), 5.12 (dd, J = 13.3, 5.0 Hz, 1H), 5.01 (s, 2H), 4.37 (ddd, J = 30.3, 15.5, 9.1 Hz, 6H), 4.26- |
| | | 4.19 (m, 2H), 4.02 (s, |
| | | 1H), 3.93 (s, 3H), 3.73 |
| | | (d, J = 30.6 Hz, 3H), |
| | | 3.59 (s, 4H), 3.51 (d, |
| | | J = 7.0 Hz, 3H), 3.39 (s, |
| | | 3H), 3.24 (s, 4H), 2.96- |
| | | 2.77 (m, 2H), 2.68 (s, |
| | | 3H), 2.58 (s, 1H), 2.41 |
| | | (s, 1H), 2.33 (s, 1H), |
| | | 2.14 (s, 2H), 2.03-1.70 |
| | | (m, 25H), 1.63 (dd, J = |
| | | 14.6, 7.0 Hz, 7H), 1.47 |
| | | (s, 2H), 0.96-0.84 (m, |
| | | 10H), 0.82 (d, J = 6.7 |
| | | Hz, 4H), 0.75 (t, J = 7.4 |
| | | Hz, 4H). |
| 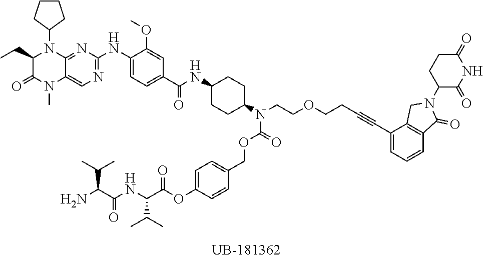 | (2S)-4-((((1S,4R)-4-(4- ((R)-8-cyclopentyl-7- ethyl-5-methyl-6-meth- yl-5-oxo-6-oxo-5,6,6, 7,8-tetrahydropteridin 2-yl-2-yl)-amino)-3- methoxybenzamido)- cyclohexyl-2-((4-(4-(2- (2-(3,6-dioxopiperidin- 3-yl)-1-oxoisooctanol- 4-yl-4-ethyl-3-methyl)- 1-yl-1-yl)oxyethycar- bamoyl)oxymethyl- methylmethyl 2-phenyl 2,2-(S)-2-amino-3-meth- ylbutylamino)-3-meth- | MS[M/2 + 1]⁺ = 604.1; ¹H NMR (400 MHz, DMSO-d₆) δ 8.42 (d, J = 8.4 Hz, 2H), 7.89 (s, 1H), 7.82 (d, J = 1.5 Hz, 1H), 7.68 (d, J = 7.6 Hz, 1H), 7.57 (d, J = 10.7 Hz, 2H), 7.51-7.35 (m, 5H), 7.06 (d, J = 8.5 Hz, 2H), 5.18-5.01 (m, 3H), 4.46-4.27 (m, 4H), 4.22 (dd, J = 7.6, 3.6 Hz, 1H), 4.03 (s, 1H), 3.93 (s, 3H), 3.78 (s, 1H), 3.60 (s, 2H), 3.53 (d, J = 6.4 Hz, 2H), 3.43 (d, J = 6.1 |
| | ylbutyrate White solid | Hz, 2H), 3.24 (s, 3H), |
| | compound (yield 41%) | 3.01-2.82 (m, 1H), 2.63- |
| | | 2.55 (m, 1H), 2.22 (dd, |
| | | J = 13.3, 6.9 Hz, 2H), |
| | | 1.90 (d, J = 5.7 Hz, |
| | | 11H), 1.77 (s, 5H), 1.59 |
| | | (d, J = 7.4 Hz, 5H), 1.48 |
| | | (s, 2H), 1.01 (dd, J = |
| | | 6.7, 2.1 Hz, 6H), 0.90 |
| | | (d, J = 6.8 Hz, 3H), 0.84 |
| | | (d, J = 6.8 Hz, 3H), 0.76 |
| | | (t, J = 7.4 Hz, 3H). |

The synthesis of compounds UB-181398 and UB-181399 was similar to that of UB-181355

### Synthesis of Compound UB-181355

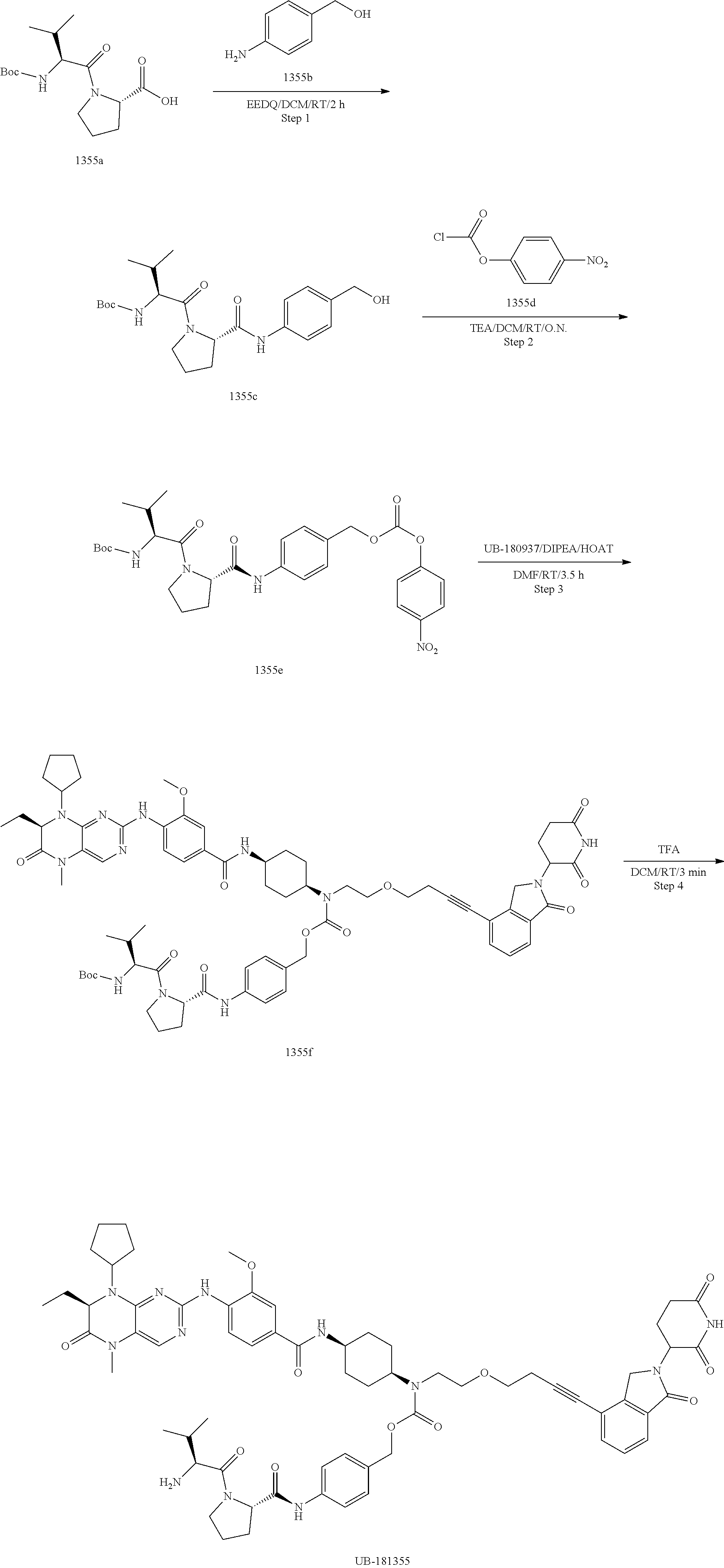

### Step 1: 1355c

### Tert-butyl ((S)-1-((S)-2-((4-(hydroxymethyl)phenyl)carbamoyl)pyrrolidin-1-yl)-3-methyl-1-oxobut-2-yl)carbamate

To a 250 mL round bottom flask was added Compound 1355a (4.0 g, 12.72 mmol), 60 mL of dichloromethane, EEDQ (6.3 g, 25.45 mmol), and Compound 1355b (3.1 g, 25.45 mmol), and the reaction solution was stirred at room temperature for 2 hours. After the removal of solvent, the residue was purified by rapid chromatography (eluent: EtOAc:PE=0-50%) to obtain desired product compound 1355c as white solid (5.4 g, 100% yield). LCMS [M+H]⁺=420.1.

### Step 2: 1355e

### Tert-butyl ((S)-3-methyl-1-((S)-2-((4-(((4-nitrophenoxy)carbonyl)oxy)methyl)phenyl)carbamoyl)pyrrolidin-1-yl)-1-oxobut-2-yl)carbamate

To a 100 mL round flask was added 1355c (2.15 g, 5.13 mmol), DCM (50 mL), triethylamine (777.7 mg, 7.7 mmol), and 1355d (1.55 g, 7.9 mmol), and the reaction solution was stirred overnight at room temperature. The reaction mixture was concentrated and the residue was purified by silica gel chromatography (eluent: EA:PE=0-33%) to afford compound 1355e (1.9 g, yield 63.0%) as yellow solid. LCMS [M-56+1]⁺=529.1.

### Step 3: 1355f

### 4-((S)-1-((S)-2-((t-butyloxycarboryl)amino)-3-methylbutyryl)pyrrolidin-2-formamido)benzyl ((1S,4R)-4-(4—(((R)-8-cyclopentyl-7-ethyl-5-methyl-6-oxo-5,6,7,8-tetrahydropterin-2-yl)amino)-3-met hoxybenzamido)cyclohexyl) (2-(4-(2-(2,6-dioxopiperidin-3-yl)-1-oxoisoindolin-4-yl)but-3-yn-1-yl)oxy)ethyl)carbamate

To a solution of UB-180937 (2.33 g, 2.71 mmol) in DMF (20 mL) was added DIPEA (1.0 g, 8.13 mmol), and stirred for 0.5 hours. Then 1355e (1.9 g, 3.25 mmol) and HOAT (0.37 g, 2.71 mmol) were added and the reaction solution was stirred at room temperature for 3 hours. The reaction solution was concentrated and the residue was purified by silica gel chromatography (eluent: DCM: DCM/MeOH (10/1)=0-54%) to afford compound 1355f (3.3 g, 93.0% yield) as white solid. LCMS [M+H]⁺=[(M-100)*1/2+H]⁺=603.2.

### Step 4: UB-181355

### 4-((S)-1-((S)-2-amino-3-methylbutyryl)pyrrolidin-2-formamido)benzyl ((1S,4R)-4-(4-(((R)-8-cyclopentyl-7-ethyl-5-methyl-6-oxo-5,6,7,8-tetrahydropterin-2-yl)amino)-3-met hoxybenzamido) (2-((4-(2-(2,6-dioxopiperidin-3-yl)-1-oxoisooctanol-4-yl)but-3-yn-1-yl)oxy)ethyl)carbamate

1355f (500 mg, 0.38 mmol) was dissolved in DCM (4 mL), and added with TFA (2 mL), and the reaction solution was stirred for 3 min at room temperature. The reaction solution was added with 60 mL of diethyl ether, stirred for 5 min, and filtered. The filter cake was dissolved in acetonitrile, and then the mixture was purified by reversed-phase chromatography (eluent: acetonitrile: 0.2% aqueous acetic acid=10%-20%-35%), and lyophilizated to give UB-181355 (238 mg, 51.6% yield) as white solid. LCMS [M*1/2+H]⁺=603.2. ¹H NMR (400 MHz, DMSO) δ 10.07 (s, 1H), 8.42 (d, J=8.4 Hz, 1H), 7.90 (d, J=5.7 Hz, 1H), 7.82 (d, J=1.6 Hz, 1H), 7.68 (d, J=7.5 Hz, 1H), 7.56 (dd, J=9.1, 4.8 Hz, 4H), 7.50-7.38 (m, 3H), 7.31 (t, J=9.5 Hz, 2H), 5.12 (dd, J=13.3, 5.1 Hz, 1H), 5.01 (s, 2H), 4.47 (dd, J=8.1, 5.4 Hz, 1H), 4.42-4.18 (m, 4H), 4.02 (s, 1H), 3.93 (s, 3H), 3.81-3.68 (m, 2H), 3.64-3.47 (m, 7H), 3.24 (s, 3H), 2.97-2.84 (m, 1H), 2.67 (dd, J=3.7, 1.8 Hz, 2H), 2.17 (d, J=6.1 Hz, 1H), 2.06-1.69 (m, 19H), 1.68-1.38 (m, 7H), 0.96 (d, J=6.7 Hz, 3H), 0.88 (d, J=6.6 Hz, 3H), 0.76 (t, J=7.4 Hz, 3H).

### Synthesis of Compound UB-181376

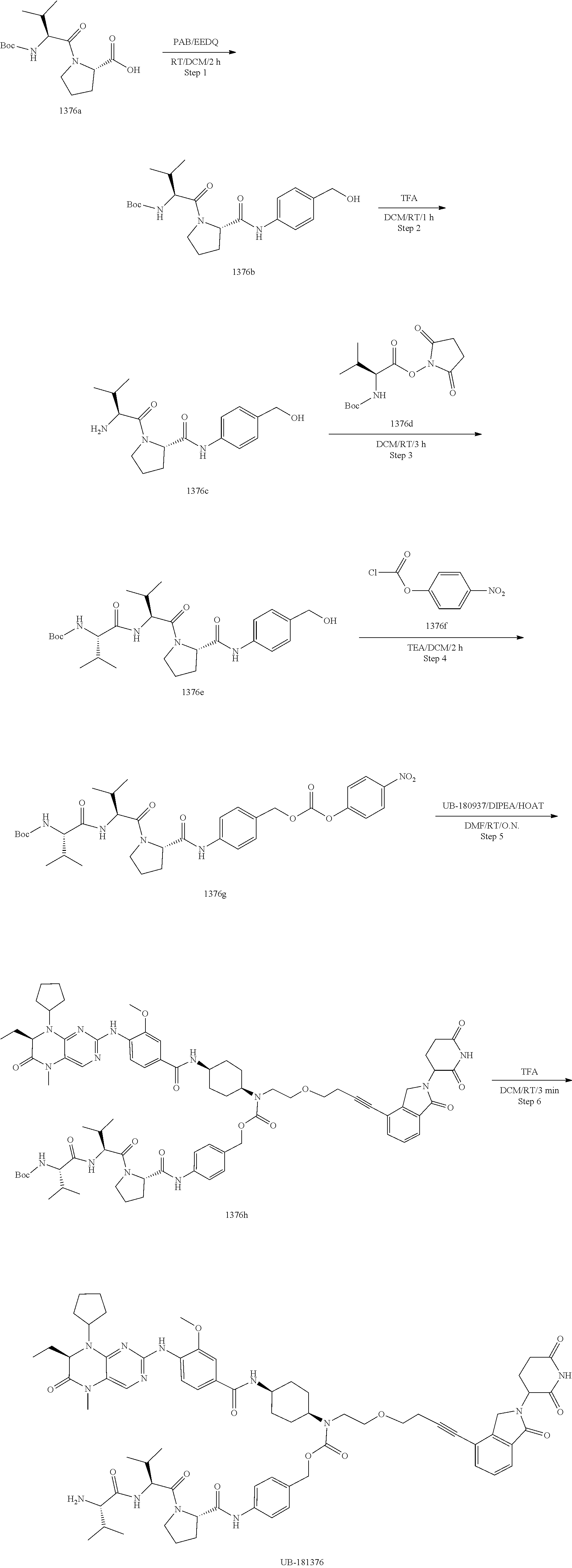

### Step 1: 1376b

### ((S-1-((S-2-((4-(hydroxymethyl)phenyl)carbamoyl)pyrrolidin-1-yl)-3-methyl-1-oxobutan-2-yl)carbamate

To a 250 mL reaction bottle was added 1376a (4.0 g, 12.72 mmol), PAB (3.1 g, 25.45 mmol), EEDQ (6.3 g, 25.45 mmol), and dichloromethane (30.0 mL). The mixture was stirred at room temperature for 2 hours. Then the mixture was concentrated and the residue was purified by chromatography (eluent: PE:EA=0-60%) to afford compound 1376b (5.0 g, yield 93.7%) as yellow solid. LCMS [M+H]⁺=420.2; [M+H-56]⁺=364.2.

### Step 2: 1376d

### (S)-1-(L-propionyl)-N—(4-(hydroxymethyl)phenyl)pyrrolidin-2-carboxamide

To a 100 mL reaction bottle was added 1376c (3 g, 7.17 mmol), trifluoroacetic acid (3 mL), and dichloromethane (12.0 mL) at room temperature. The mixture was stirred at room temperature for 1 hour. The reaction solution was directly spin-dried and used in the next step 1376c (crude) without purification. LCMS: [M+97]⁺=416; TM [M+H]⁺=320

### Step 3: 1376f

### Tert-butyl ((S)-1-((S-1-((S)-2-((4-(hydroxymethyl)phenyl)carbamoyl)pyrrolidin-1-yl)-3-methyl-1-oxobutan-2-yl)amino)-3-methyl ester-1-chlorobutan-2-yl)

To a 250 mL reaction bottle was added 1376c (2.2 g, 6.80 mmol), triethylamine (2.1 g, 20.40 mmol), and dichloromethane (20.0 mL) and 1376d (3.2 g, 10.20 mmol) at room temperature. The mixture was reacted at room temperature for 2 hours. The reaction solution was spin-dried and added with ammonia (30 mL) in methanol, and the mixture was stirred for 10 min. Then the mixture was concentrated and the residue was purified by chromatography (eluent: EA:PE=0-100%) to afford product compound 1376b (3.5 g, yield: 94.6%) as yellow solid. LCMS [M+H]⁺=519.2

### Step 4: 1376g

### tert-Butyl ((S)-3-methyl-1-((S-3-methyl-1-((S)-2-((4-(((4-nitrophenoxy)carbonyl)oxy)methyl)phenyl)carbamoyl)pyrrolidin-1-yl)-1-oxobutan-2-yl)amino)-1-oxobutan-2-yl)

To a 250 mL reaction bottle was added 1376e (1.7 g, 3.25 mmol), triethylamine (0.7 g, 6.55 mmol), 1376g (1.3 g, 6.55 mmol) and dichloromethane (20.0 mL) at room temperature. The mixture was reacted at room temperature for 2 hours. Then the mixture was concentrated and the residue was purified by chromatography (eluent: PE:EA=0-50%) to afford compound 1376 g (858 mg, yield: 39%) as yellow solid. LCMS [M+H]⁺=684.2

### Step 5: 1376h

### 4-((S)-1-((tert-butoxycarbonyl)-L-propionyl-L-propionyl)pyrrolidin-2-formamido)benzyl ((1S,4R)-4-(4-(((R)-8-cyclopentyl-7-ethyl-5-methyl-6-oxo-5,6,7,8-tetrahydropterin-2-yl)amino)-3-met hoxybenzamido) (2-((4-(2-(2,6-dioxopiperidin-3-yl)-1-oxoisoindolin-4-yl)but-3-yn-1-yl (oxy)ethyl)carbamate

To a 100 mL reaction bottle was added UB-180937 (794.6 mg, 0.92 mmol), DMF (10.0 mL), and DIPEA (357.3 mg, 2.77 mmol) at room temperature, and the mixture was stirred at room temperature for 30 min. To the above mixture was added 1376g (752.1 mg, 1.10 mmol), HOAT (125.8 mg, 0.92 mmol) and stirred at 30° C. overnight. Then the mixture was concentrated and the residue was purified by chromatography (eluent: DCM: DCM/MeOH (10/1)=0-80%) to afford compound 1376h as yellow solid (793 g, yield: 61.1%) LCMS [M/2+H]⁺=702.9; [(M-100)/2+1]⁺=652.9

### Step 6: UB-181376

### 4-(S)-1-((S)-2-((S)-2-amino-3-methylbutylamino)-3-methylbutyryl)pyrrolidin-2-formamido)benzyl((1S,4R)-4-(4-(((R)-8-cyclopentyl-7-ethyl-5-methyl-6-oxo-5,6,7,8-tetrahydropterin-2-yl)amino)-3-methoxybenzamido)(2-(4-(2-(2,6-dioxopiperidin-3-yl)-1-oxoisooctanol-4-yl)but-3-yn-1-yl)oxy)ethyl)carbamate

To a 100 mL reaction bottle was added 1376h (686.9 mg, 0.50 mmol), dichloromethane (4.0 mL) and trifluoroacetic acid (2.0 mL) at room temperature. The mixture was stirred at room temperature for 3 minutes. Upon completion of the reaction, the reaction solution was added with diethyl ether, and then filtered. The filter cake was dissolved in acetonitrile and the mixture was purified by chromatography (eluent: acetonitrile: 0.2% aqueous acetic acid=10%−20%−35%) to give 1376 as white solid (332.1 mg, yield: 38%). LCMS [M/2+H]⁺=653. ¹H NMR (400 MHz, DMSO) δ 11.09 (s, 1H), 10.13 (s, 1H), 8.48 (d, J=8.4 Hz, 1H), 8.09 (d, J=8.8 Hz, 1H), 7.96 (d, J=5.6 Hz, 1H), 7.87 (s, 1H), 7.74 (d, J=7.5 Hz, 1H), 7.66-7.60 (m, 4H), 7.52 (dd, J=16.0, 8.3 Hz, 2H), 7.46 (s, 1H), 7.35 (d, J=8.4 Hz, 2H), 5.81 (d, J=0.9 Hz, 1H), 5.18 (dd, J=13.3, 5.0 Hz, 1H), 5.07 (s, 2H), 4.51-4.33 (m, 6H), 4.32-4.25 (m, 2H), 4.08 (s, 1H), 3.99 (s, 3H), 3.86 (s, 2H), 3.82 (s, 2H), 3.66 (d, J=6.3 Hz, 5H), 3.57 (d, J=6.1 Hz, 3H), 3.30 (s, 4H), 3.06 (d, J=4.7 Hz, 1H), 2.95 (s, 1H), 2.79 (s, 1H), 2.74 (s, 3H), 2.64 (d, J=2.4 Hz, 1H), 2.46 (d, J=12.1 Hz, 1H), 2.25-2.16 (m, 2H), 2.11-1.77 (m, 22H), 1.75-1.61 (m, 6H), 1.52 (d, J=6.4 Hz, 3H), 1.40 (d, J=6.6 Hz, 1H), 0.99 (d, J=6.6 Hz, 3H), 0.95-0.88 (m, 8H), 0.82 (d, J=7.0 Hz, 5H)

### Synthesis of Compound UB-181354

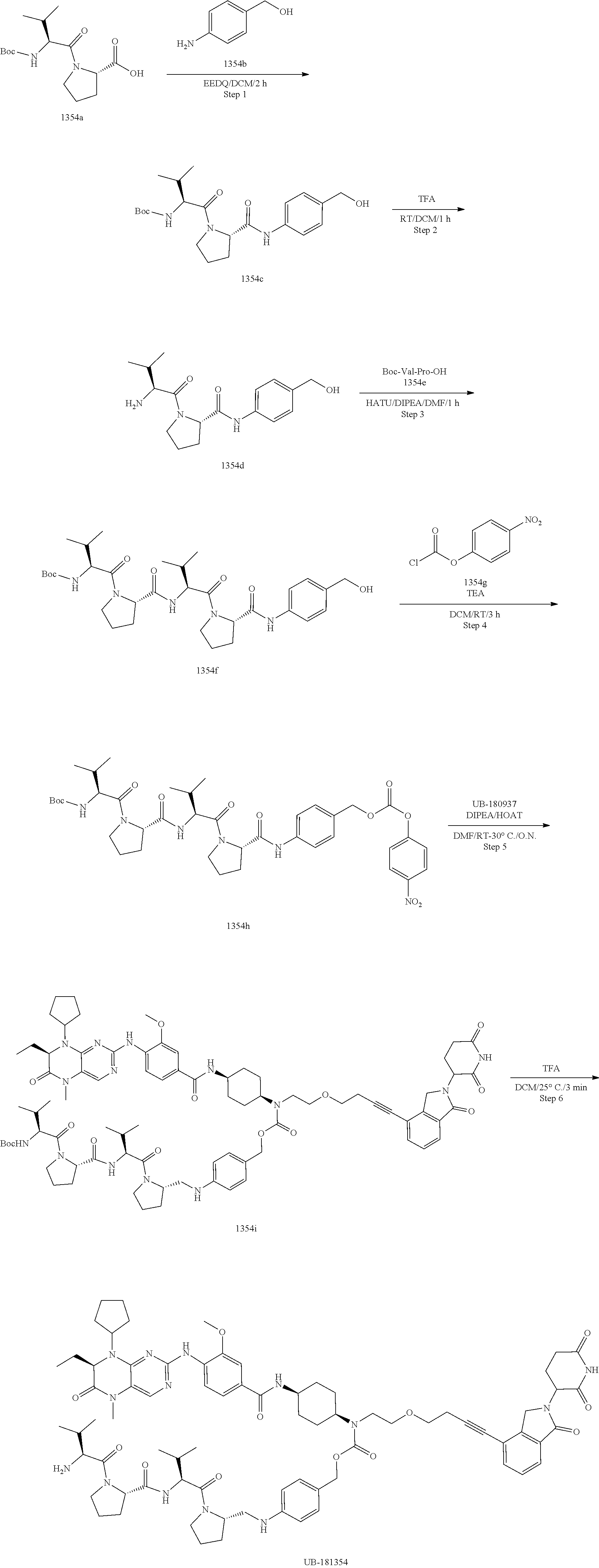

### Step 1: 1354c

### Tert-butyl ((S)-1-((S)-2-((4-(hydroxymethyl)phenyl)carbamoyl)pyrrolidin-1-yl)-3-methyl-1-oxobutan-2-yl)carbamate

To a 250 mL reaction bottle was added 1354a (2.0 g, 6.26 mmol), 1354b (1.6 g, 12.72 mmol), EEDQ (3.1 g, 12.72 mmol), and dichloromethane (30.0 mL). The mixture was stirred at room temperature for 2 hours. Then the mixture was concentrated and the residue was purified by chromatography (eluent: EA:PE=0-60%) to afford compound 1354c as yellow solid (3.0 g, yield: >100%). LCMS [M+H]⁺=420.2; [M+H−56]⁺=364.2.

### Step 2: 1354d

### (S)-1-(L-propionyl)-N—(4-(hydroxymethyl)phenyl)pyrrolidin-2-carboxamide

To a 100 mL reaction bottle was added 1354c (3 g, 7.17 mmol), trifluoroacetic acid (3 mL), and dichloromethane (12.0 mL) at room temperature. The mixture was stirred at room temperature for 1 hour. The reaction solution was directly spin-dried and used in the next step without purification. LCMS [M+97]⁺=416; TM [M+H]⁺=320

### Step 3: 1354f

### tert-Butyl ((S)-1-((S)-2-((S)-1-((S)-2-(4-(hydroxymethyl)phenyl)carbamoyl)pyrrolidin-1-yl)-3-methyl-1-oxobutan-2-yl)carbamoyl)pyrrolidin-1-yl)-3-methyl-1-chlorobutan-2-yl)

To a 250 mL reaction bottle was added 1354d (1.8 g, 5.79 mmol), HATU (4.4 g, 11.58 mmol), DIPEA (2.2 g, 17.37 mmol) and DMF (20.0 mL) at room temperature, and 1354e (2.7 g, 8.35 mmol) was added slowly under stirring. The mixture was reacted at room temperature for 1 hour. The reaction solution was spin-dried and added with ammonia (30 mL) in methanol. The mixture was stirred for 10 min and then concentrated and the residue was purified by chromatography (eluent: PE:EA=0-100%) to afford product compound 1354f (crude) as yellow solid. LCMS [M+H]⁺=616.4

### Step 4: 1354h

### tert-Butyl ((S)-3-methyl-1-((S)-2-((S)-3-methyl-1-) ((S)-2-(((4-((4-nitrophenoxy)carbonyl)oxy)methyl)phenyl)carbamoyl)pyrrolidin-1-yl)-1-oxobutan-2-yl)carbamoyl)pyrrolidinyl-1-oxybutan-1-yl)carbamate

To a 250 mL reaction bottle was added 1354f (3.6 g, 5.79 mmol), triethylamine (1.2 g, 11.58 mmol), UB-181354g (2.3 g, 11.58 mmol) and dichloromethane (20.0 mL) at room temperature. The mixture was reacted at room temperature for 3 hours. Then the mixture was concentrated and the residue was purified by chromatography (eluent: PE/EA=0/100%) to afford compound 1354h (1.38 g, yield: 30.5%) as yellow solid. LCMS [M+H]⁺=781.4; [M+H−100]⁺=681.3

### Step 5: 1354i

### tert-Butyl ((S)-3-methyl-1-((S)-2-((S)-3-methyl-1-) ((S-2-)(((4-((4-nitrophenoxy)carbonyl)oxy)methyl)phenyl)carbamoyl)pyrrolidin-1-yl)-1-oxobutan-2-yl)carbamoyl)pyrrolidinyl-1-methyl)-1-oxobutane-

To a 250 mL reaction bottle was added 0937 (1006.2 mg, 1.17 mmol), DMF (20.0 mL), and DIPEA (452.8 mg, 3.51 mmol) at room temperature, and stirred at room temperature for 30 min. To the above mixture was added 1354h (1326.6 mg, 1.70 mmol), HOAT (159.2 mg, 1.17 mmol) and stirred at 30° C. overnight. Then the mixture was concentrated and the residue was purified by chromatography (eluent: DCM:DCM/MeOH (10/1)=0-80%) to afford compound 1354i as yellow solid (1.22 g, yield: 69.43%). LCMS [M/2+H]⁺=751.5; [(M-100)/2+H]⁺=701.5

### Step 6: UB-181354

### 4-((S)-1-(L-propionyl-L-prolyl-L-propionyl)pyrrolidin-2-formamido)benzyl ((1S,4R)-4-(4-(((R)-8-cyclopentyl-7-ethyl-5-methyl-6-oxo-5,6,7,8-tetrahydropterin-2-yl)amino)-3-met hoxybenzamido) (2-((4-(2-(2,6-dioxopiperidin-3-yl)-1-oxoisooctanol-4-yl)but-3-yn-1-yl)oxy)ethyl)carbamate

To a 100 mL reaction bottle was added UB-181354i (195.1 mg, 0.13 mmol), dichloromethane (4.0 mL) and trifluoroacetic acid (2.0 mL) at room temperature. The mixture was stirred at room temperature for 3 minutes. Upon completion of the reaction, the reaction solution was added with diethyl ether, and then filtered. The filter cake was dissolved in acetonitrile and the mixture was purified by chromatography (eluent: acetonitrile: 0.2% aqueous acetic acid=10%−20%−35%) to give UB-181354 as white solid (21.7 mg, yield: 65.1%). LCMS [M/2+H]⁺=701.5. ¹H NMR (400 MHz, DMSO) δ 11.31-10.65 (m, 1H), 10.04 (d, J=15.3 Hz, 1H), 8.42 (d, J=8.4 Hz, 1H), 7.90 (s, 1H), 7.85 (d, J=8.3 Hz, 1H), 7.82 (d, J=1.5 Hz, 1H), 7.68 (d, J=7.5 Hz, 1H), 7.60-7.54 (m, 4H), 7.46 (dd, J=16.8, 8.8 Hz, 2H), 7.39 (s, 1H), 7.29 (d, J=8.5 Hz, 2H), 5.12 (dd, J=13.3, 5.0 Hz, 1H), 5.01 (s, 2H), 4.37 (ddd, J=30.3, 15.5, 9.1 Hz, 6H), 4.26-4.19 (m, 2H), 4.02 (s, 1H), 3.93 (s, 3H), 3.73 (d, J=30.6 Hz, 3H), 3.59 (s, 4H), 3.51 (d, J=7.0 Hz, 3H), 3.39 (s, 3H), 3.24 (s, 4H), 2.96-2.77 (m, 2H), 2.68 (s, 3H), 2.58 (s, 1H), 2.41 (s, 1H), 2.33 (s, 1H), 2.14 (s, 2H), 2.03-1.70 (m, 25H), 1.63 (dd, J=14.6, 7.0 Hz, 7H), 1.47 (s, 2H), 0.96-0.84 (m, 10H), 0.82 (d, J=6.7 Hz, 4H), 0.75 (t, J=7.4 Hz, 4H).

### Synthesis of Compound UB-181362

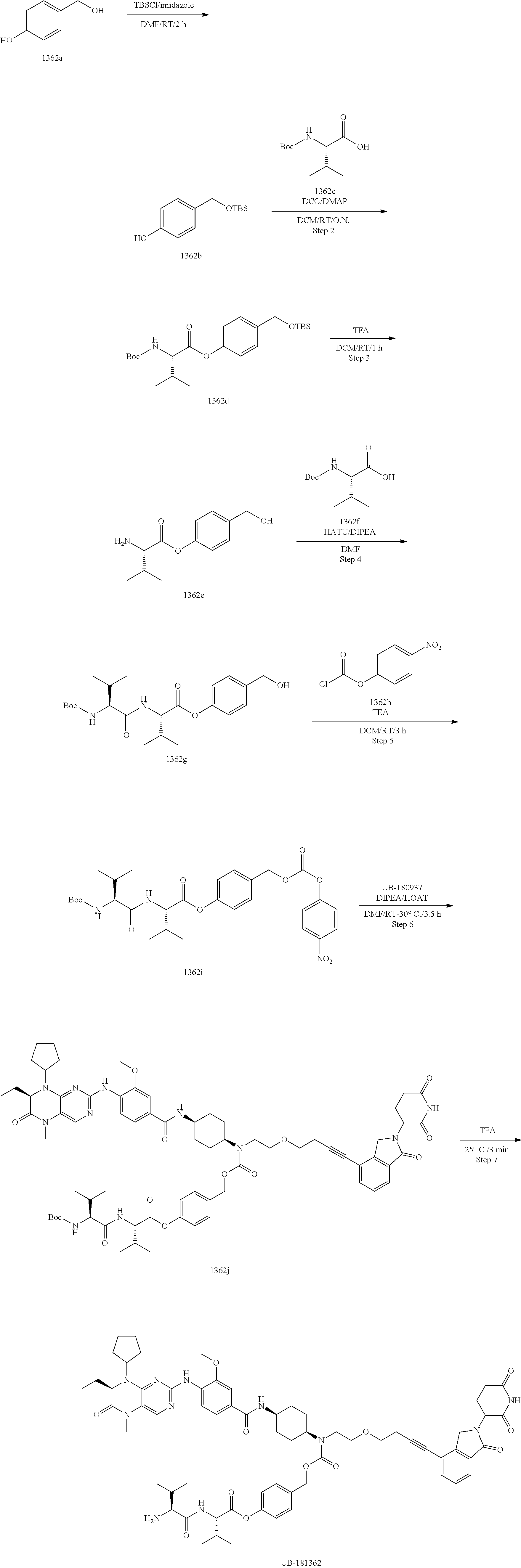

### Step 1: 1362b

### 4-(((tert-butyldimethylsilyl)oxy)methyl)phenol

To a solution of compound 1362a (600 mg, 4.83 mmol) in DMF (8 mL) was successively added imidazole (822 mg, 12.09 mmol), and TBSCl (874 mg, 0.806 mmol). The mixture was reacted for 2 hours at ordinary temperature. Upon completion of the reaction, the mixture was added with water and extracted three times with ethyl acetate. The organic phase was concentrated under reduced pressure, and the residue was separated by column chromatography (eluent: PE: EA=0-10%) to give compound 1362b (900 mg, 78% yield) as colorless liquid. LCMS [M+H]'⁰=107.1.

### Step 2: 1362d

### (S)-4-((tert-butyldimethylsilyl)oxy)methyl)phenyl 2-((tert-butoxycarbonyl)amino)-3-methylbutyrate

To a solution of compound 1362b (300 mg, 1.261 mmol) in DCM (8 mL) was successively added 1362c (410 mg, 1.891 mmol) and DCC (779 mg, 3.783 mmol) as well as DMAP (153 mg, 1.261 mmol). The mixture was reacted at ordinary temperature overnight. The reaction solution was concentrated, and the residue was separated by column chromatography (eluent: PE: EA=0-10%) to give compound 1362d (400 mg, 73% yield) as colorless liquid. LCMS [M+H]⁺=337.2.

### Step 3: 1362e

### (S)-4-(Hydroxymethyl)phenyl 2-amino-3-methylbutyrate

To a solution of compound 1362d (400 mg, 0.915 mmol) in DCM (5 mL) was successively added TFA (2 mL, 1.830 mmol), and reacted at ordinary temperature for 1 h. The reaction solution was concentrated and dissolved in methanol, and the mixture was subjected to reverse phase column to give 1362e (200 mg, 98% yield). LCMS [M+H]⁺=224.1.

### Step 4: 1362g

### (S)-4-(Hydroxymethyl)phenyl 2-(S)-2-((tert-butoxycarbonyl)amino)-3-methylbutylamino)-3-methylbutyrate

To a solution of compound 1362e (161 mg, 0.743 mmol) in DMF (5 mL) was added HATU (423 mg, 1.114 mmol) and DIPEA (191 mg, 1.486 mmol). The resulting mixture was reacted at ordinary temperature for 15 min. 1362f (174 mg, 0.780 mmol) was added, and the reaction was stirred at ordinary temperature for 1 h. When finished, the reaction was added with water and extracted three times with ethyl acetate. The organic phase was concentrated under reduced pressure, and the residue was separated by column chromatography (eluent: PE:EA=0-50%) to give compound 1362g (160 mg, 40% yield) as white solid. LCMS [M+H]⁺=367.2

### Step 5: 1362i

### (2S)-4-(((((1S,4R)-4-(4-((R)-8-cyclopentyl-7-ethyl-5-methyl-6-methyl-5-oxo-6-oxo-5,6,6,7,8-tetrahydropteridin 2-yl-2-yl)-amino)-3-methoxybenzamido)cyclohexyl-2-((4-(4-(2-(2-(3,6-dioxopiperidin-3-yl)-1-oxoisooctanol-4-yl-4-ethyl-3-methyl)-l-yl-1-yl)oxyethylcarbamoyl)oxymethyl 2-phenyl 2,2-(S)-2-((tert-butoxycarbonyl)amino)-3-methylbutylamino)-3-methylbutyrate

To a solution of compound 1362g (100 mg, 0.237 mmol) in DCM (5 mL) was added Et₃N (48 mg, 0.473 mmol) and 1362h (95 mg, 0.473 mmol). The resulting mixture was reacted at ordinary temperature for 1 h. The reaction solution was concentrated, and the residue was separated by column chromatography (eluent: PE: EA=0-20%) to give compound 1362i (70 mg, 50% yield) as white solid. LCMS [M-100+H]⁺=488.2

### Step 6: 1362j

### 1-((1R)-1-(3-Chloro-4-(7-fluoro-1-hydroxyisoquinolin-8-yl)phenyl)-2-hydroxyethyl)-3-(2-ethynylthiazol-4-yl)urea

To a solution of compound UB-937 (78 mg, 0.092 mmol) in DMF (3 mL) was added DIPEA (35 mg, 0.275 mmol) and stirred at ordinary temperature for 0.5h. HoAt (12 mg, 0.093 mmol) and 1362i (70 mg, 0.119 mmol) were added. The mixture was reacted for 2 hours at ordinary temperature. After completion of the reaction, the mixture was added with water and extracted three times with ethyl acetate. The organic phase was concentrated under reduced pressure, and the residue was separated by column chromatography (eluent: DCM: DCM/MeOH (10/1)=0-50%) to give compound 1362j (89 mg, 74% yield) as white solid. LCMS [M-100/2+H]⁺=608.1.

### Step 7: UB-181362 (V4524-040)

### (2S)-4-(((((1S,4R)-4-(4-((R)-8-cyclopentyl-7-ethyl-5-methyl-6-methyl-5-oxo-6-oxo-5,6,6,7,8-tetrahydropteridin 2-yl)-amino)-3-methoxybenzamido)cyclohexyl-2-((4-(4-(2-(2-(3,6-dioxopiperidin-3-yl)-1-oxoisooctanol-4-yl-4-ethyl-3-methyl)-1-yl-1-yl)oxyethylcarbamoyl)oxymethyl 2-phenyl 2,2-(S)-2-amino)-3-methylbutylamino)-3-methylbutyrate

To a solution of compound 1362j (100 mg, 0.076 mmol) in DCM (2 mL) was added TFA (1 mL) and the reaction solution was stirred at ordinary temperature for 3 min. The reaction solution was added with diethyl ether (40 mL) to precipitate solid. The solid was filtered under reduced pressure, and the filter cake was dissolved with acetonitrile, and the solution was separated by reverse phase column chromatography (eluent: eluent: acetonitrile:0.2% aqueous acetic acid=10%-30%) to obtain compound UB-181362 (38 mg, 41% yield) as white solid. LCMS [M/2+H]⁺=604.1. ¹H NMR (400 MHz, DMSO-d₆) δ 8.42 (d, J=8.4 Hz, 2H), 7.89 (s, 1H), 7.82 (d, J=1.5 Hz, 1H), 7.68 (d, J=7.6 Hz, 1H), 7.57 (d, J=10.7 Hz, 2H), 7.51-7.35 (m, 5H), 7.06 (d, J=8.5 Hz, 2H), 5.18-5.01 (m, 3H), 4.46-4.27 (m, 4H), 4.22 (dd, J=7.6, 3.6 Hz, 1H), 4.03 (s, 1H), 3.93 (s, 3H), 3.78 (s, 1H), 3.60 (s, 2H), 3.53 (d, J=6.4 Hz, 2H), 3.43 (d, J=6.1 Hz, 2H), 3.24 (s, 3H), 3.01-2.82 (m, 1H), 2.63-2.55 (m, 1H), 2.22 (dd, J=13.3, 6.9 Hz, 2H), 1.90 (d, J=5.7 Hz, 11H), 1.77 (s, 5H), 1.59 (d, J=7.4 Hz, 5H), 1.48 (s, 2H), 1.01 (dd, J=6.7, 2.1 Hz, 6H), 0.90 (d, J=6.8 Hz, 3H), 0.84 (d, J=6.8 Hz, 3H), 0.76 (t, J=7.4 Hz, 3H).

### Synthesis of Compound UB-181313

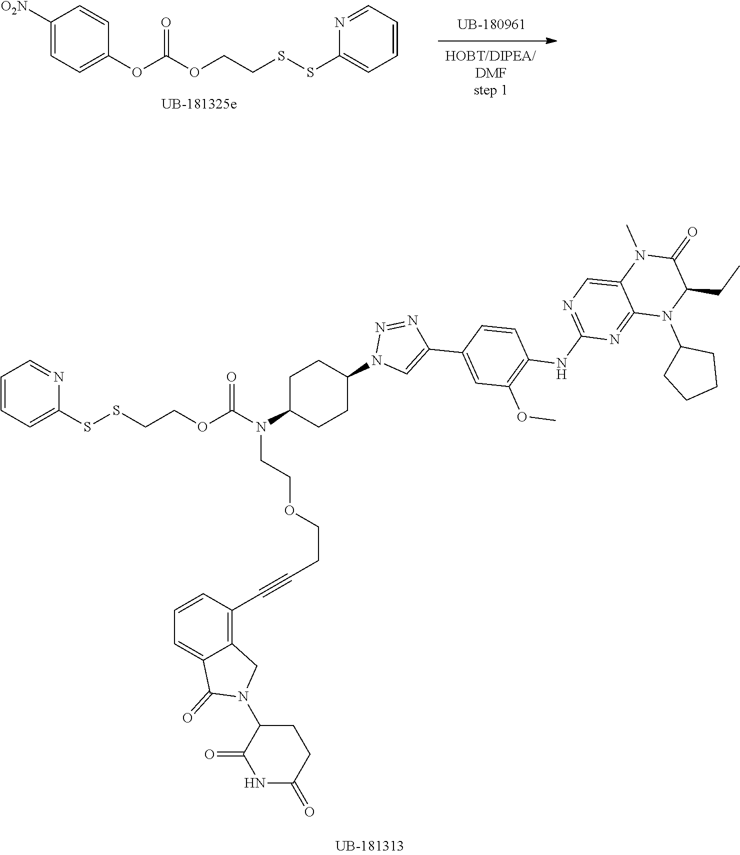 [SMILES: O=C(OCCSSc1ccccn1)Oc1ccc([N+](=O)[O-])cc1 ; CC[C@@H]1C(=O)N(C)c2cnc(Nc3ccc(-c4cn([C@H]5CC[C@@H](N(CCOCCC#Cc6cccc7c6CN(C6CCC(=O)NC6=O)C7=O)C(=O)OCCSSc6ccccn6)CC5)nn4)cc3OC)nc2N1C1CCCC1]

### Step 1: UB-181313

Compound UB-181325e (20 mg, 0.06 mmol) was dissolved in DMF (3 mL), and then UB-180961 (40 mg, 0.0.05 mmol), HOBT (8 mg, 0.06 mmol) and DIEA (15 mg, 0.11 mmol) were added The mixture was reacted at room temperature for 16 hours. The reaction solution was subjected to preparative chromatograph to obtain target product UB-181313 (35.6 mg, yield 57%) as white solid. LCMS [M+H]⁺=1098.3

### Synthesis of Compound UB-181332

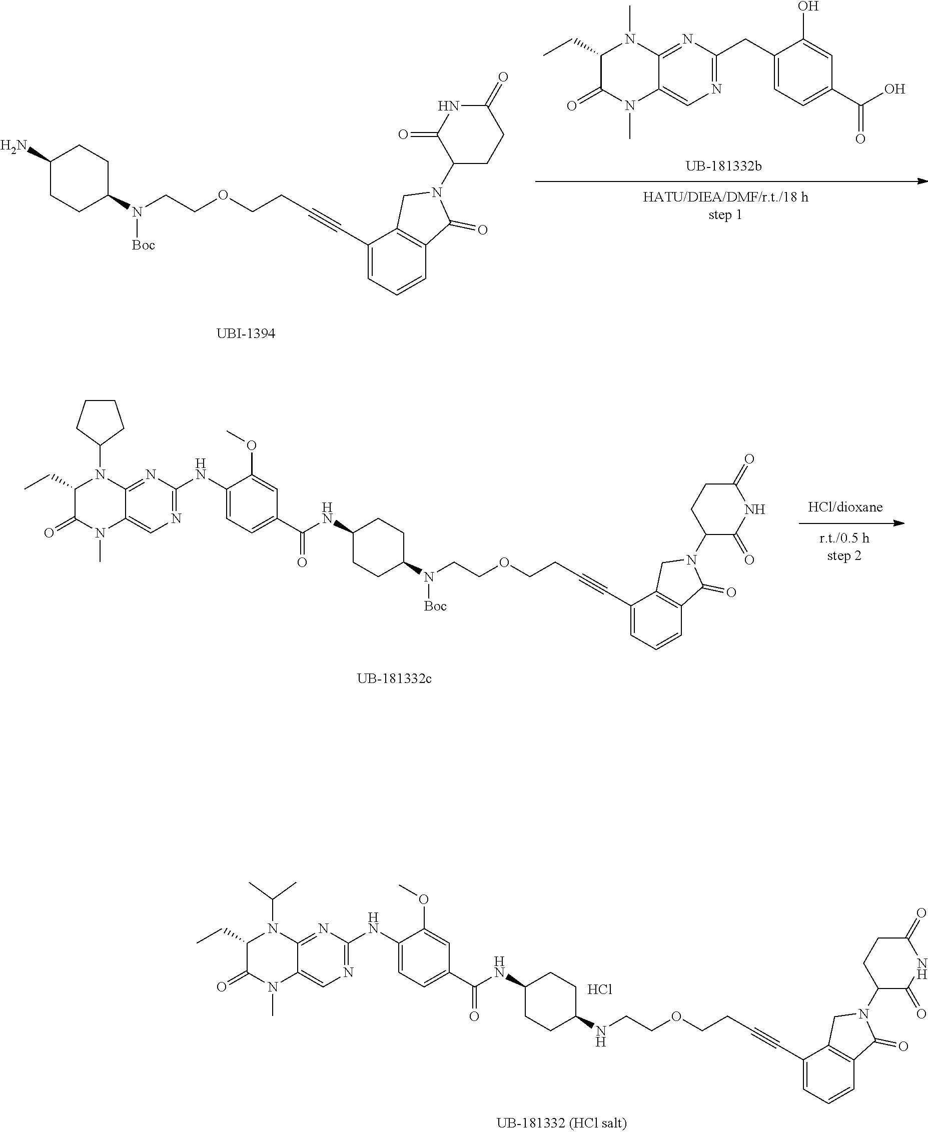 [SMILES: CC(C)(C)OC(=O)N(CCOCCC#Cc1cccc2c1CN(C1CCC(=O)NC1=O)C2=O)[C@H]1CC[C@@H](N)CC1 ; CC[C@H]1C(=O)N(C)c2cnc(Cc3ccc(C(=O)O)cc3O)nc2N1C ; CC[C@H]1C(=O)N(C)c2cnc(Nc3ccc(C(=O)N[C@H]4CC[C@@H](N(CCOCCC#Cc5cccc6c5CN(C5CCC(=O)NC5=O)C6=O)C(=O)OC(C)(C)C)CC4)cc3OC)nc2N1C1CCCC1 ; CC[C@H]1C(=O)N(C)c2cnc(Nc3ccc(C(=O)N[C@H]4CC[C@@H](NCCOCCC#Cc5cccc6c5CN(C5CCC(=O)NC5=O)C6=O)CC4)cc3OC)nc2N1C(C)C ; Cl]

### Step 1: UB-181332c

Compound UB-181332b (1.38 g, 3.261 mmol) was in dissolved in DMF (9 mL), and added with HATU (2478.26 mg, 6.522 mmol) and DIEA (1261.96 mg, 9.783 mmol). The mixture was stirred at room temperature for 1 hour. The mixture was added with UBI-1394 (1.8 g, 3.261 mmol) and stirred at room temperature for 12 hours. The mixture was purified by silica gel chromatography (DCM/MeOH=0˜80%) to obtain UB-181332c (1.658 g, 53% yield) as white solid. LCMS [M+H]+=961.0

### Step 2: UB-181332

Compound UB-181332c (1.658 g, 1.727 mmol) was dissolved in DCM (5 mL), and added with HCl/dioxane (2.16 mL), and the mixture was stirred at room temperature for 2 hours. The reaction solution was washed with Et2O (10 mL*3) and the mixture was filtered. The solid was collected to give product UB-181332 (1.27 g, 85.5% yield) as white solid. LCMS [M+H]+=861.0

### Synthesis of Compound UB-181333

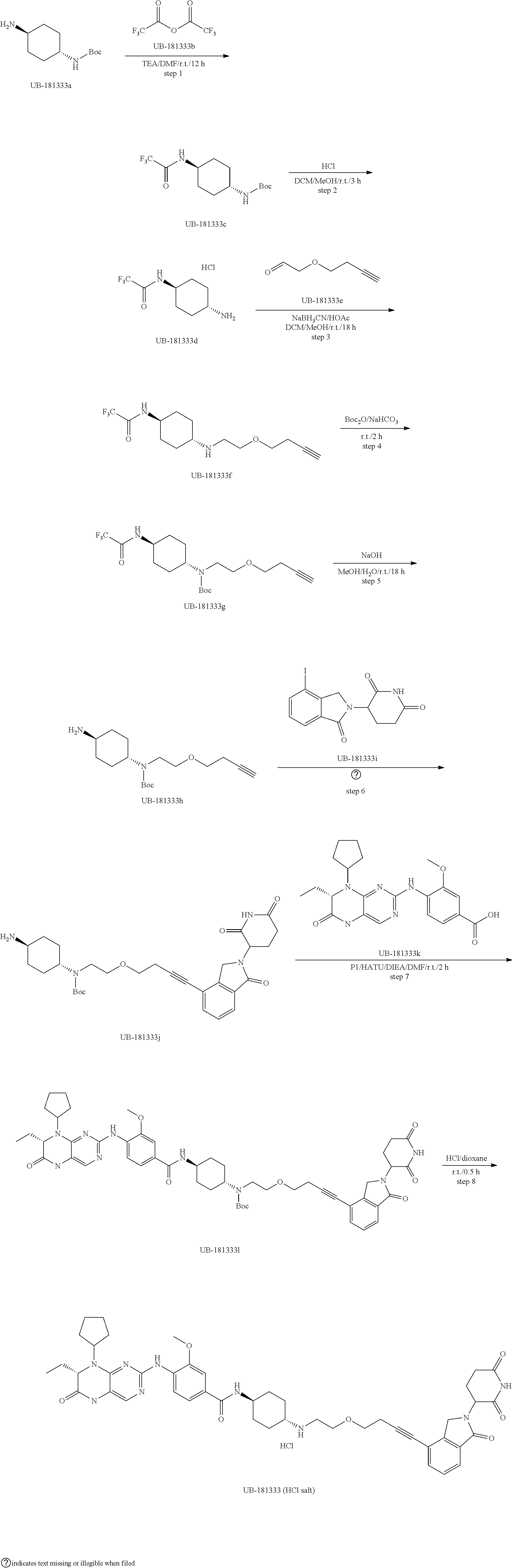

Compound UB-1333a (5 g, 23.4 mmol) was dissolved i DMF) mL), and added with UB-181333b (9.8 g, 46.8 mmol) and TEA (7.9 g, 70.2 mmol). The mixture was stirred at room temperature for 12 hours. The mixture was added with water, filtered and the filtrate was concentrated to give product UB-181333c (6.79 g, 93.7% yield) as white solid. LCMS [M+H]⁺=311.0.

### Step 2: UB-181333c

Compound UB-181332c (6.79 g, 21.881 mmol) was dissolved in DCM (30 mL), added with HCl/dioxane solution (27 mL), and the mixture was stirred at room temperature for 1 hour. The reaction solution was washed with diethyl ether (10 mL*3) and the mixture was filtered. The solid was collected to give product UB-181333d (4.79 g) as white solid. LCMS [M+H]⁺=211.0

### Step 3: UB-181333f

Compound UB-181333d (4.79 g, 19.354 mmol) was dissolved in dichloromethane and methanol, and added with UB-181333e (3.25 g, 29 mmol) and AcOH (0.5 mL). The mixture was stirred for 6 h, and then added with NaBH₃CN (3.6 g, 58 mmol) and stirred for 3 hours at room temperature. NaHCO3 was added to the mixture. The organic layer was added with DCM and dried over Na₂SO₄, filtered and the filtrate was concentrated. The residue was purified by silica gel chromatography (MeOH/DCM-NH₃H₂O=0 to 50%) to afford product UB-181333f as white oil (2.6 g, 42% yield). LCMS [M+H]+=307.0

### Step 4: UB-181333g

Compound UB-181333f (2.6 g, 8.497 mmol) was dissolved in THF (10 mL), and added with Boc₂O (3.7 g, 16.993 mmol) and NaHCO3 (1.4 g, 16.993 mmol). The mixture was stirred at room temperature for 2 hours. EA was added to the mixture. The organic layer was dried over Na₂SO₄, filtered and the filtrate was concentrated. The residue was purified by silica gel chromatography (PE/EA=0˜30%) to afford product UB-181333g as white oil (2.667 g, 38.7% yield). LCMS [M+H]+=407.0

### Step 5: UB-181333h

Compound B-181333g (6 g, 14 mmol) was dissolved MeOH (20 mL), and added with NaOH (1.18 g, 29.5 mmol) and H₂O (3 mL). The mixture was stirred at room temperature for 12 hours. The mixture was washed with DCM. The organic layer was dried over Na₂SO₄, filtered and the filtrate was concentrated. The residue was purified by silica gel chromatography (DCM/MeOH=0-75%) to obtain UB-181333h (4.295 g, 47.7% yield) as white solid. LCMS [M+H]+=311.0

### Step 6: UB-181333j

Compound UB-181333h (4.295 g, 13.855 mmol) was dissolved in DMF (40 mL), the mixture was added with CuI (526.48 mg, 2.771 mmol), pd(pph₃)₂Cl₂ (485.61 mg, 0.693 mmol), TEA (1399.34 mg, 13.8548 mmol) and UB-181333i (5126.29 mg, 13.855 mmol), and the mixture was stirred at 85° C. for 2 h under nitrogen protection. The mixture was purified by silica gel chromatography (DCM/MeOH:=0 to 80%) to obtain UB-181333hj (4.2 g, 54.8% yield) as white solid. LCMS [M+H]+=553.0

### Step 7: UB-181333i

Compound UB-181333k (1.23 g, 2.9 mmol) was dissolved in DMF (10 mL), and added with HATU (2.2 g, 5.8 mmol) and DIEA (1.12 g, 8.7 mmol). The mixture was stirred at room temperature for 1 hour. The mixture was added with UB-181333hj (1.6 mg, 2.9 mmol) and stirred at room temperature for 12 hours. The mixture was purified by silica gel chromatography (DCM/MeOH=0˜80%) to obtain UB-181333i (980 mg, 36% yield) as white solid. LCMS [M+H]+=961.0

### Step 8: UB-181333

Compound UB-181333i (980 mg, 1.021 mmol) was dissolved in DCM (5 mL), added with HCl/dioxane (2 mL), and the mixture was stirred at room temperature for 2 hours. The reaction solution was washed with Et2O (10 mL*3) and the mixture was filtered. The solid was collected to give product UB-181333 (788 mg, yield 89.8%) as white solid. LCMS [M+H]+=861.0

### Synthesis of Compound UB-181334

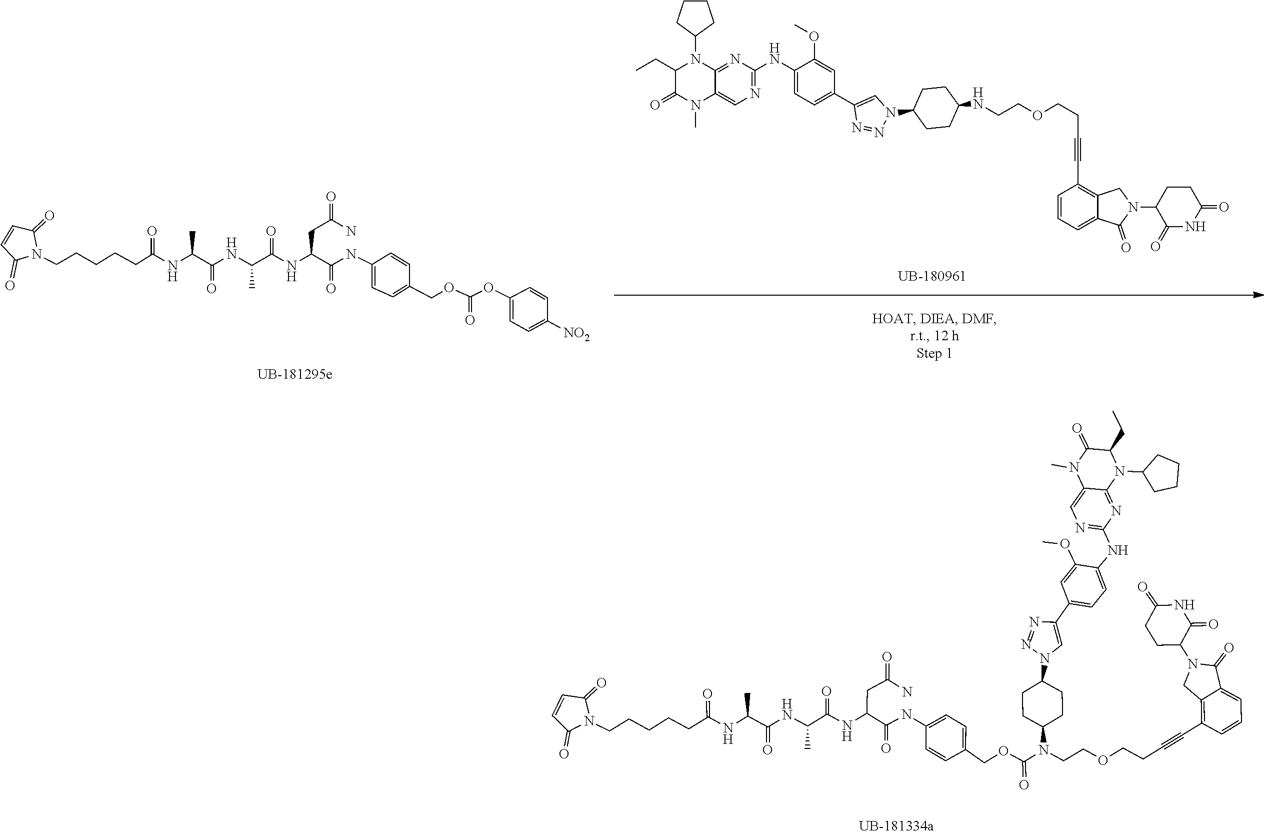

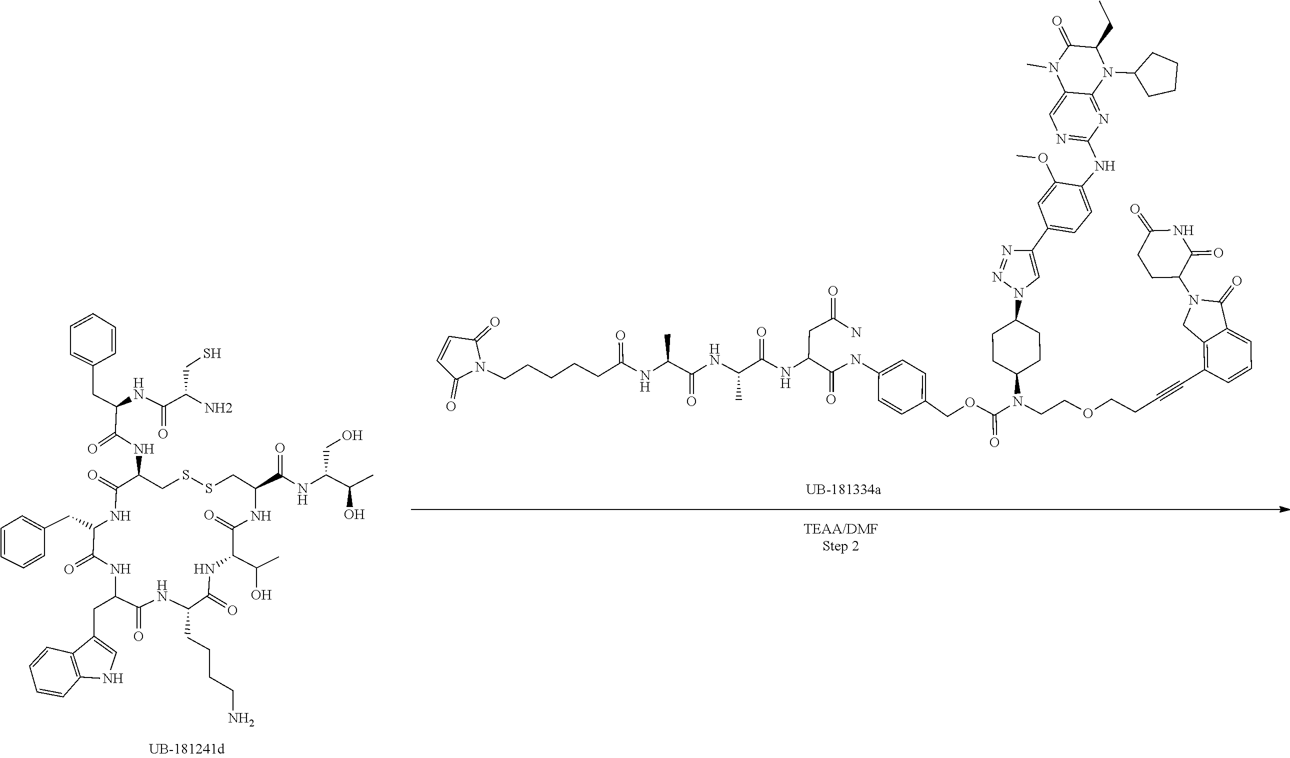

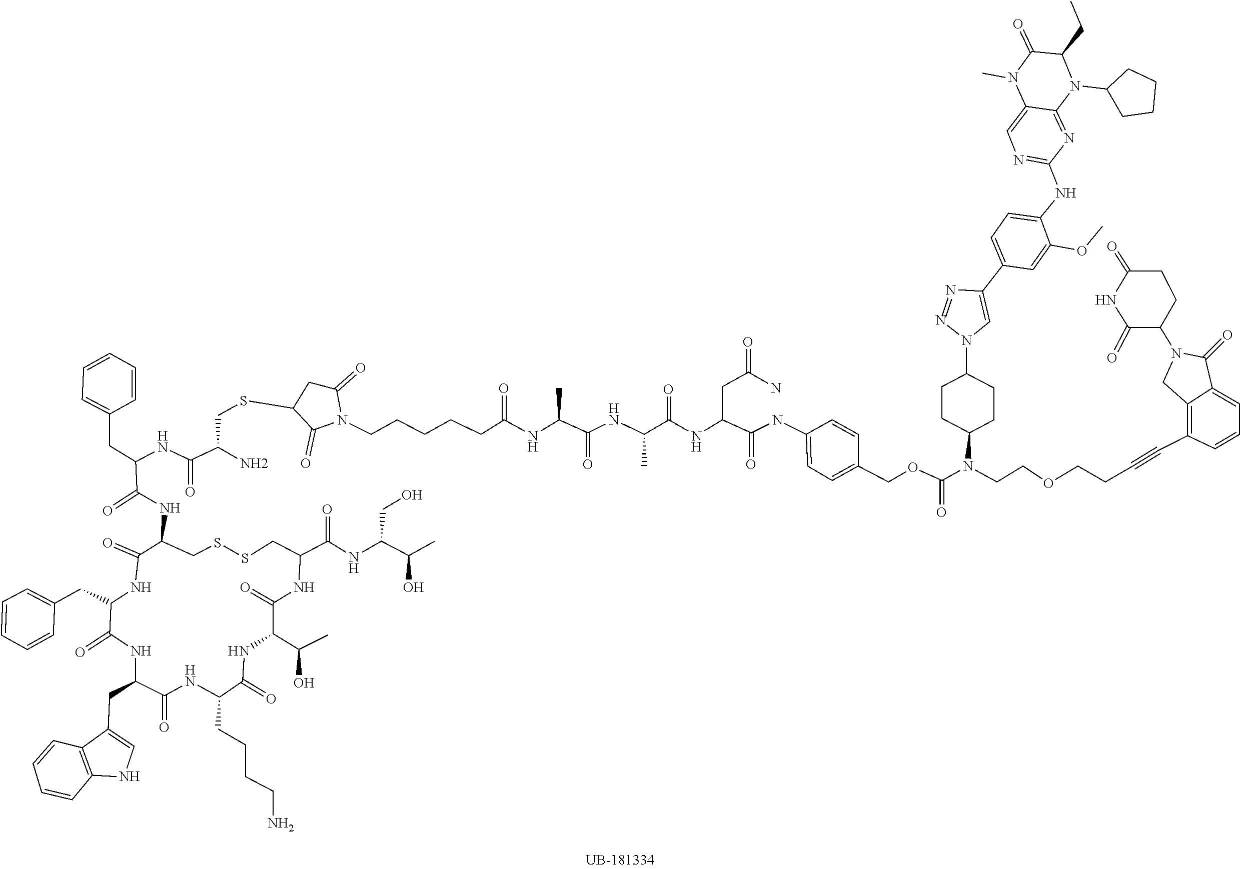

### Step 1: UB-181334

To a solution of UB-181295e (50 mg, 0.07 mmol) in DMF (3 mL) was added UB-180961 (48 mg, 0.05 mmol), HOAT (9 mg, 0.07 mmol) and DIEA (26 mg, 0.2 mmol). The reaction mixture was stirred at room temperature for 5 hours. The solution was concentrated and purified by reverse phase column (MeCN/0.5% ACOH2H₂O=20-100%, collected at 35%) to obtain UB-181334a as yellow solid (43 mg, 61% yield). LCMS [M+1]⁺=1483.6

### Step 2: UB-181334

To a solution of UB-181241d (20 mg, 0.02 mmol) in TEAA/H₂O (0.5 mL) was added a solution of UB-181334a (41 mg, 0.01 mmol) in DMF (1 ml), and the reaction mixture was stirred at room temperature for 2 hours. The solution was concentrated and purified by reverse phase column (MeCN/H₂O=20-100%) to obtain desired product as yellow solid (4 mg, 14% yield). LCMS [M/2+1]⁺=1303.2, LCMS [M/3+1]⁺=869.0

### Synthesis of Compound UB-181335

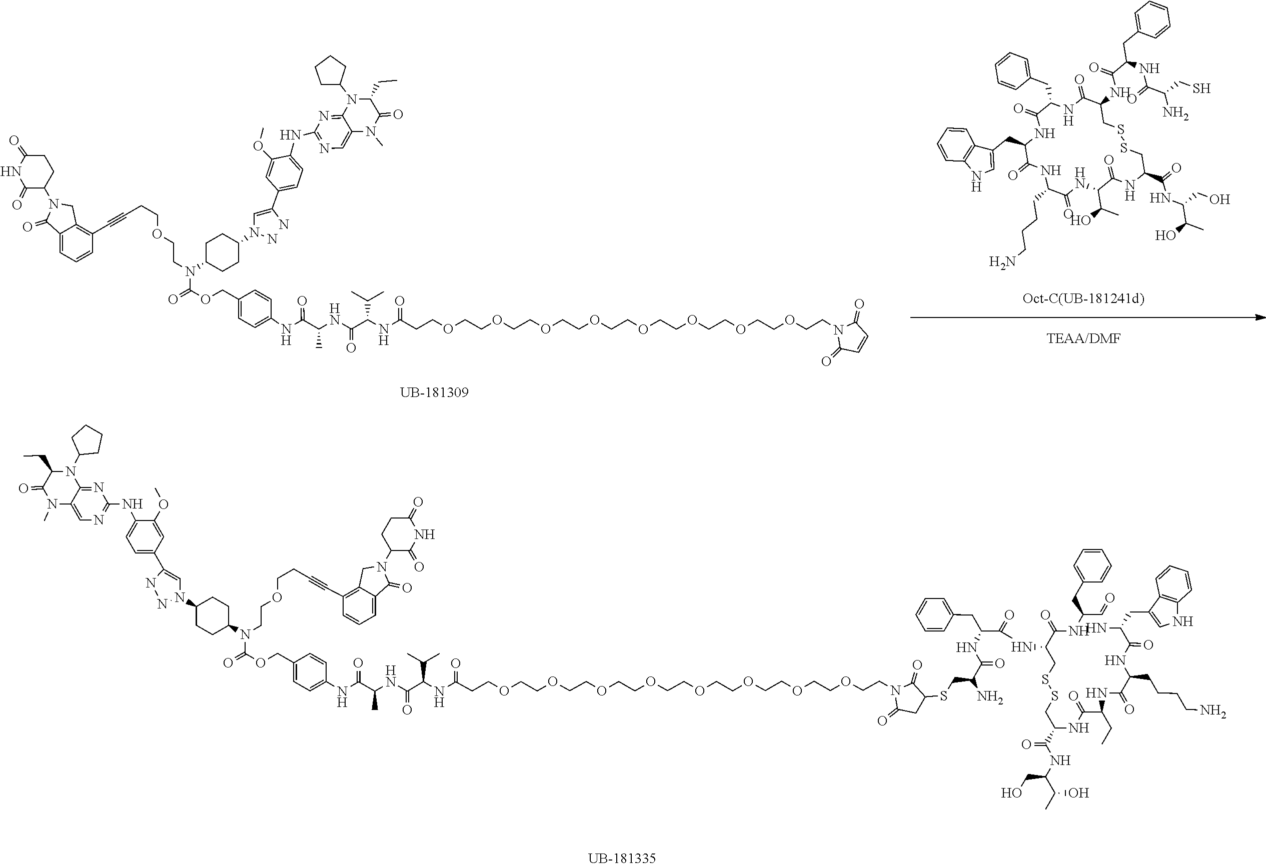

### Step 1: UB-181335

A solution of Oct-C (18 mg, 0.01 mmol) dissolved in DMF (1 ml) was added dropwise to a solution of UB-181309 (15 mg, 0.013 mmol) and TEAA (0.5 ml) at room temperature. The mixture was reacted for half an hour at room temperature. The reaction solution was purified by C-18 reversed-phase column chromatograph and medium pressure preparative chromatography, 50 mmol/l TEAA/H2O/MeCN, to obtain product V3441-139 (UB-181335, 1.5 mg, 5% yield) as yellow solid. LCMS[M/2+H]=1416.43

### Synthesis of Compound UB-181336

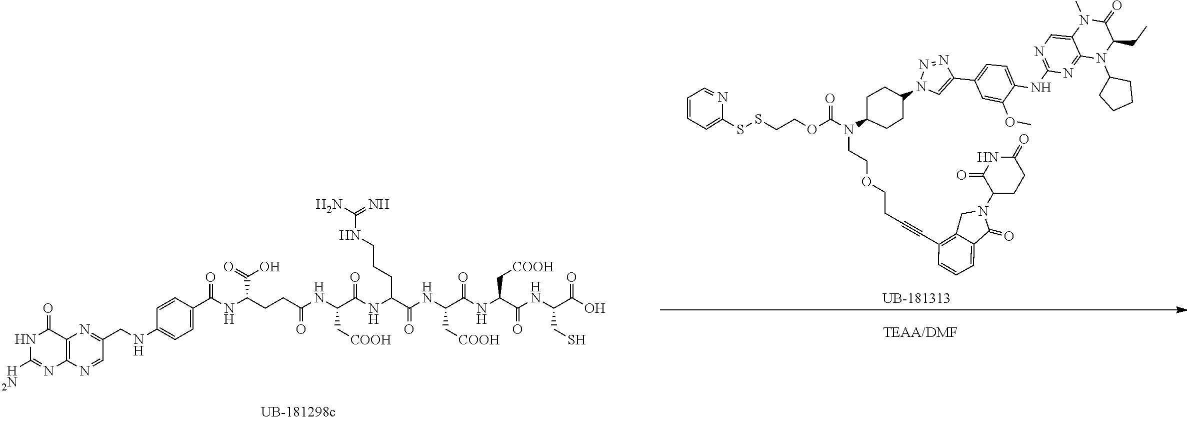

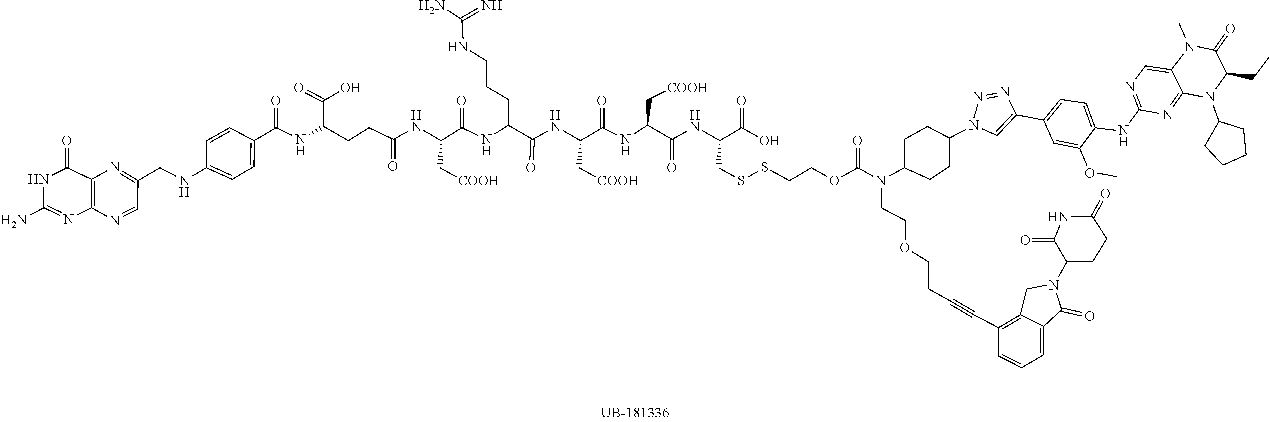

### Step 1: UB-181336

To a solution of UB-181298c (20 mg, 0.05 mmol) in TEAA/H₂O (0.5 mL) was added a solution of UB-181313 (41 mg, 0.05 mmol) in DMF (1 ml), and the reaction mixture was stirred at room temperature for 2 hours. The solution was concentrated and purified by reverse phase column chromatograph (MeCN/H₂O=20-100%) to obtain UB-181336 as yellow solid (16 mg, 70% yield). LCMS [M/2+1]⁺=1018.6, LCMS [M/3+1]⁺=679.0

### Synthesis of Compound UB-181337

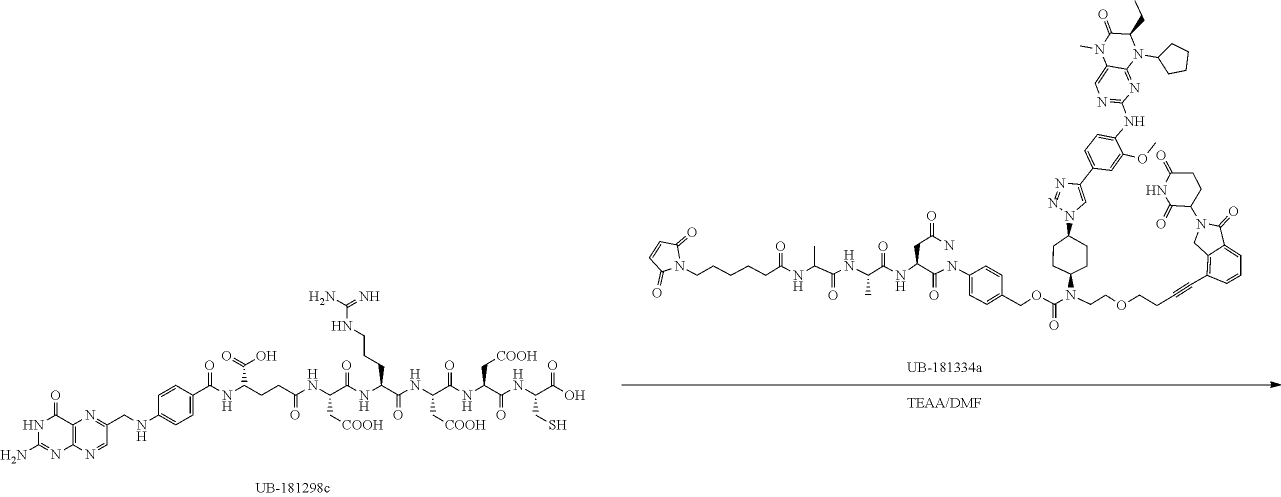

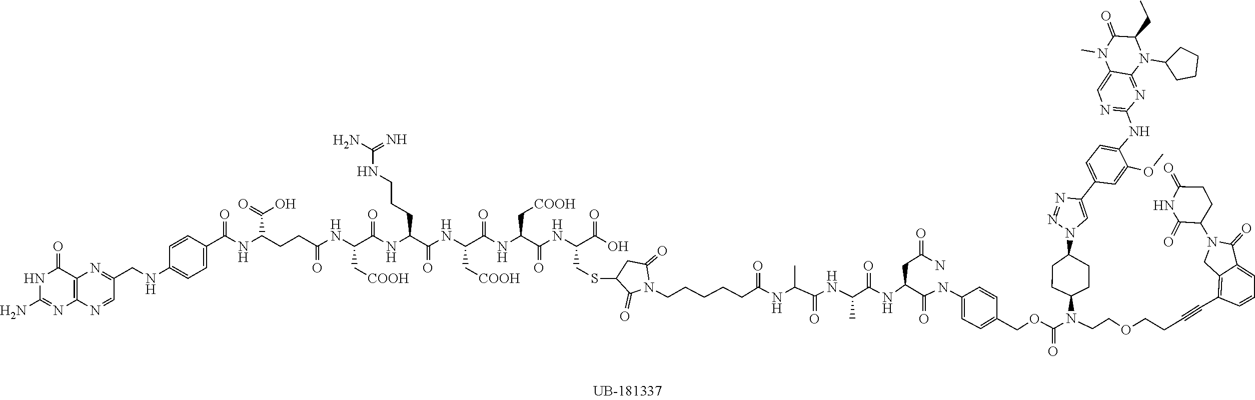

### Step 1: UB-181337

To a solution of UB-181298c (20 mg, 0.02 mmol) in TEAA/H₂O (0.5 mL) was added with a solution of UB-181334a (41 mg, 0.01 mmol) in DMF (1 ml), and the reaction mixture was stirred at room temperature for 2 hours. The solution was concentrated and purified by reverse phase column chromatograph (MeCN/H₂O=20-100%) to obtain UB-181337 as yellow solid (16 mg, 55% yield). LCMS [M/2+1]⁺=1264.8, LCMS [M/3+1]⁺=843.6

### Synthesis of Compound UB-181353

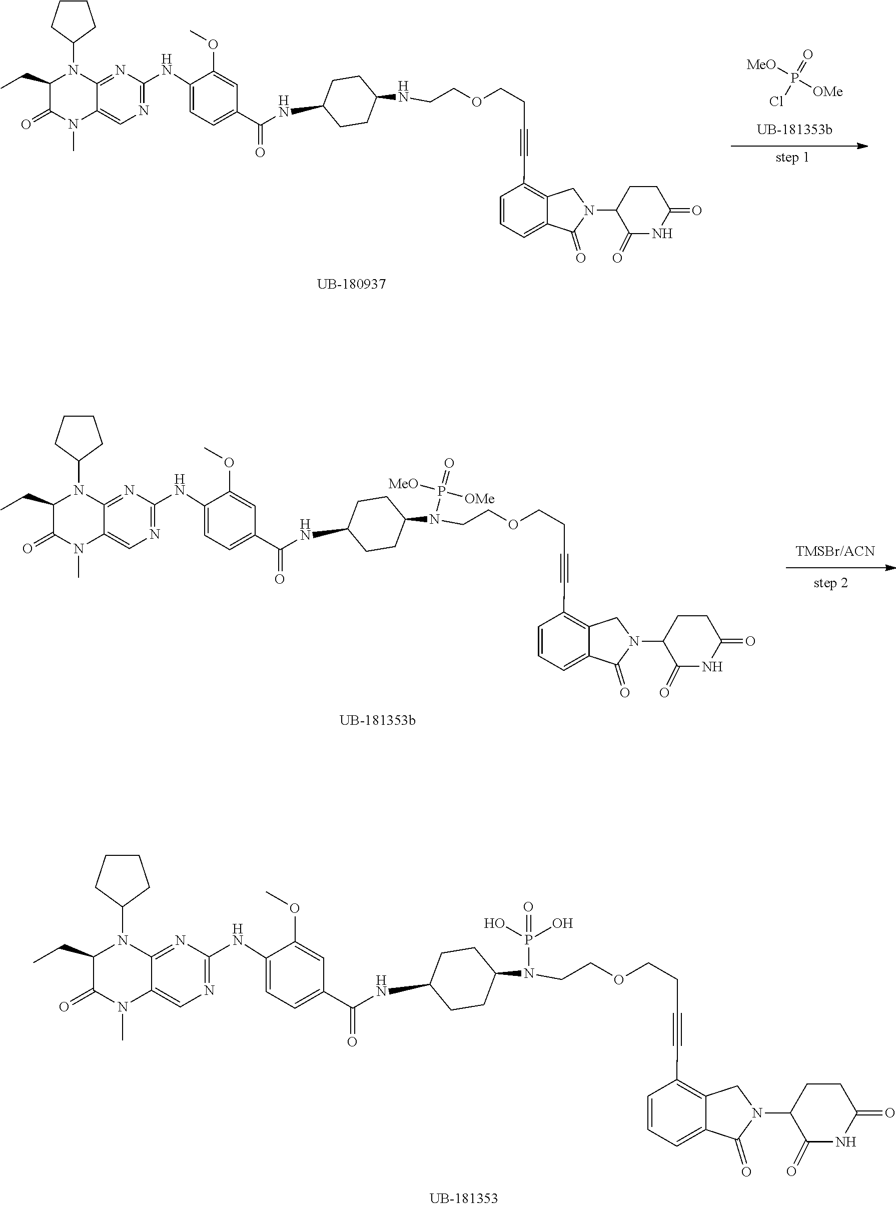 [SMILES: CC[C@@H]1C(=O)N(C)c2cnc(Nc3ccc(C(=O)N[C@H]4CC[C@@H](NCCOCCC#Cc5cccc6c5CN(C5CCC(=O)NC5=O)C6=O)CC4)cc3OC)nc2N1C1CCCC1 ; COP(=O)(Cl)OC ; CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)P(=O)(OC)OC)CC4)cc3OC)nc2N1C1CCCC1 ; CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)P(=O)(O)O)CC4)cc3OC)nc2N1C1CCCC1]

### Step 1: UB-181353b

UB-180937 (500 mg, 0.58 mmol) and DIEA (225 mg, 1.74 mmol) were dissolved in anhydrous DCM (25 mL), and the mixture was cooled to (0° C.). To a stirred solution was added dropwise dimethyl phosphate (84 mg, 0.58 mmol). 1 hour later, the solution was returned to room temperature and stirred for another 24 hours. The reaction mixture was poured into water and extracted with DCM (3×20 ml). The organic layers were combined, washed with saturated saline (2×30 ml), dried over anhydrous Na2SO4 and evaporated under reduced pressure to give crude product. The crude product was purified by reversed-phase chromatography to afford UB-181353b (260 mg, 45% yield) as white solid. LCMS: [M+1]⁺=969.2.

### Step 2: UB-181353

To a solution of UB-181353b (60 mg, 0.062 mmol) in MeCN (25 mL) was added TMSBr (0.1 mL) at 0° C. The resulting solution was stirred overnight at room temperature. Then solution was added with ice water and extracted with DCM (3×20 ml). The organic layer was dried over Na2SO4, filtered and concentrated to give crude product. The crude product was purified by washing with hot MeCN, MeOH and Et2O to give crude product. The crude product was purified by reversed-phase chromatography to give UB-181353 (22 mg, 31% yield) as white solid. LCMS: [M+1]⁺=940.3. ¹H NMR (400 MHz, DMSO) δ 11.03 (s, 1H), 8.48 (s, 2H), 8.02-7.86 (m, 2H), 7.73 (d, J=7.5 Hz, 1H), 7.64 (d, J=6.8 Hz, 1H), 7.61-7.44 (m, 3H), 7.37 (d, J=8.1 Hz, 1H), 5.17 (dd, J=13.3, 5.1 Hz, 1H), 4.47 (s, 1H), 4.35 (dd, J=18.2, 10.1 Hz, 2H), 3.86 (s, 1H), 3.79-3.60 (m, 8H), 3.47-3.38 (m, 2H), 3.20 (d, J=15.4 Hz, 6H), 3.00-2.87 (m, 1H), 2.79 (t, J=6.7 Hz, 2H), 2.63 (t, J=8.9 Hz, 1H), 2.16-2.01 (m, 3H), 2.01-1.81 (m, 6H), 1.75 (d, J=11.6 Hz, 3H), 1.60 (d, J=11.0 Hz, 2H), 1.58 (s, 2H), 1.41 (dd, J=6.8, 10.4 Hz, 2H), 0.71 (dt, J=14.2, 7.3 Hz, 3H).

### Synthesis of Compound UB-181356

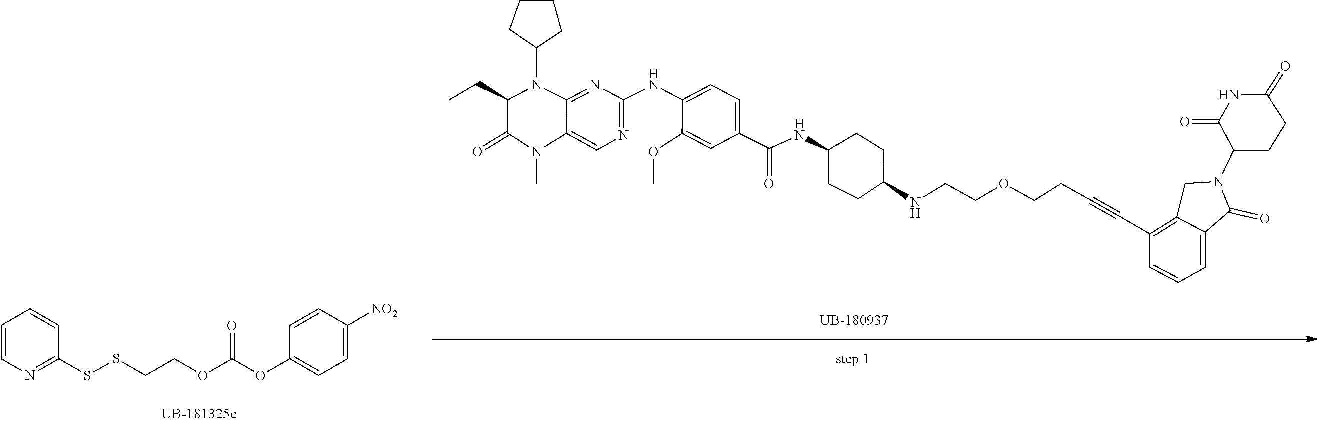

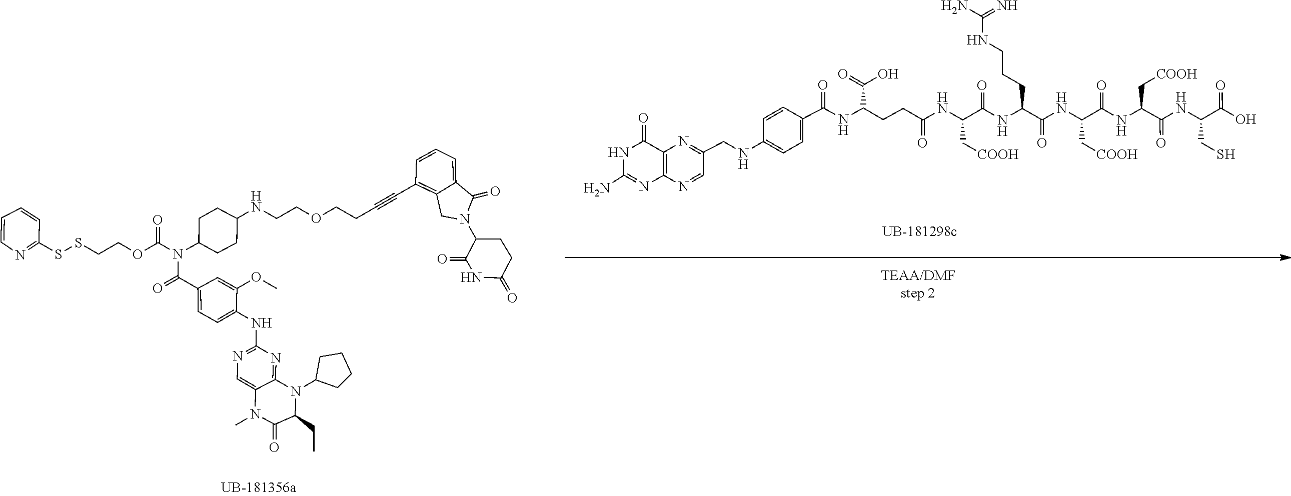

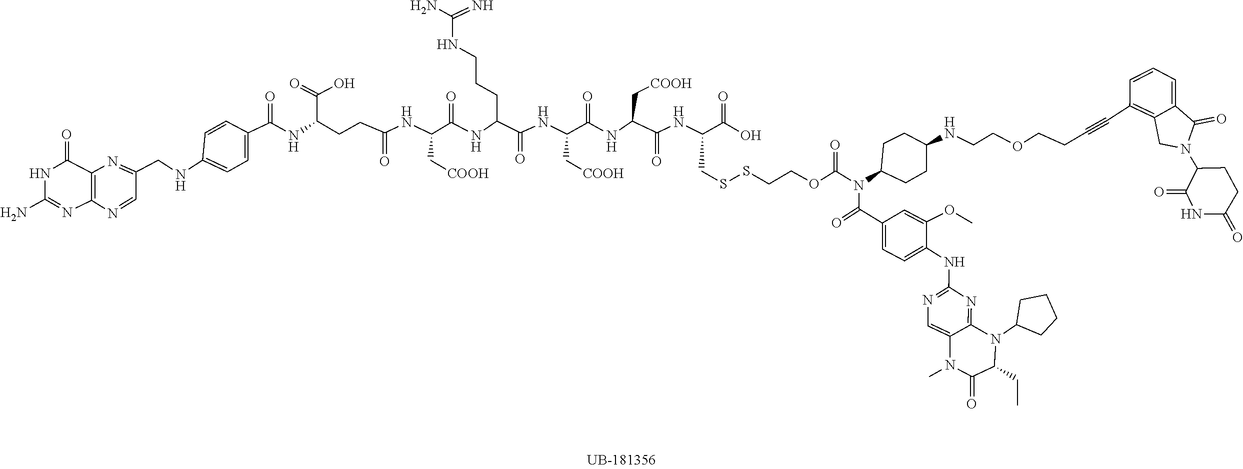 [SMILES: CC[C@@H]1C(=O)N(C)c2cnc(Nc3ccc(C(=O)N(C(=O)OCCSSC[C@H](NC(=O)[C@H](CC(=O)O)NC(=O)[C@H](CC(=O)O)NC(=O)C(CCCNC(=N)N)NC(=O)[C@H](CC(=O)O)NC(=O)CC[C@H](NC(=O)c4ccc(NCc5cnc6nc(N)[nH]c(=O)c6n5)cc4)C(=O)O)C(=O)O)[C@H]4CC[C@@H](NCCOCCC#Cc5cccc6c5CN(C5CCC(=O)NC5=O)C6=O)CC4)cc3OC)nc2N1C1CCCC1]

### Step 1: UB-181356a

To a solution of UB-181325e (50 mg, 0.14 mmol) in DMF (5 mL) was added HOBT (19 mg, 0.14 mmol), UB-180937 (98 mg, 0.11 mmol) and DIEA (37 mg, 0.28 mmol) and then the reaction mixture was stirred for 2 hours at room temperature. The reaction mixture was concentrated in vacuo and purified by silica gel chromatography (DCM/MeOH=10/1) to obtain UB-181356a (40 mg, 26.3% yield) as white solid. LCMS [M+1]⁺=1073.6

### Step 4: UB-181356

To a solution of UB-181298c (170 mg, 0.16 mmol) in TEAA/H₂O (1 mL) was added a solution of UB-181356a (113 mg, 0.11 mmol) in DMF (2 ml), and the reaction mixture was stirred at room temperature for 2 hours. The solution was concentrated and purified by TFA preparative chromatograph to obtain UB-181356 (100 mg, 47% yield) as yellow solid. LCMS [M/2+1]⁺=1004.8, LCMS [M/3+1]⁺=670.6.

### Synthesis of Compound UB-181359

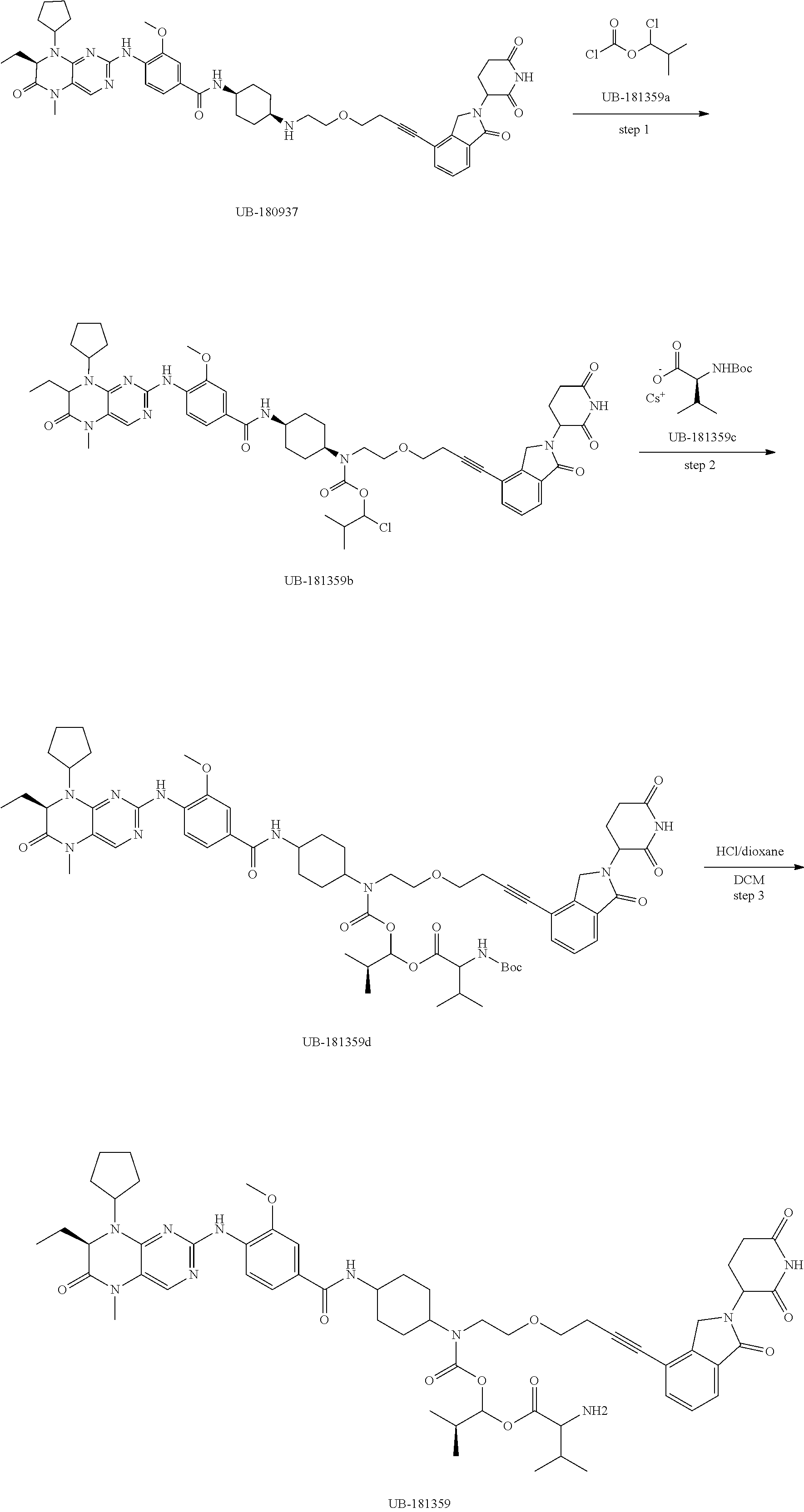

### Step 1: UB-181359b

Compound UB-180937 (200 mg, 0.23 mmol) was dissolved in DMF (5 mL) and then the mixture was cooled to −50° C. NaHMDS (85 mg, 0.47 mmol) was added dropwise to the reaction solution and reacted for 15 min. Then UB-181359a (99 mg, 0.58 mmol) was added dropwise to the reaction solution at −20° C. and the mixture was reacted for 5 min and then reacted at room temperature for 30 min. The reaction solution was quenched with 3N HCl, and then extracted with a solution of MeOH/DCM=1/10. The organic phase was concentrated, and then separated by column chromatography (methanol/dichloromethane=1/10) to obtain target product UB-181359b (250 mg, yield 100%) as brown oil. LCMS [M+H]⁺=994.8

### Step 2: UB-181359d

To a reaction solution of UB-181359b (77 mg, 0.08 mmol) dissolved in DMF (3 mL) was added UB-181359c (41 mg, 0.12 mmol) and TBAI (43 mg, 0.12 mmol), and the mixture was reacted at room temperature overnight. The reaction solution was separated by column chromatography (methanol/dichloromethane=1/10) to obtain target product UB-181359d (40 mg, yield 44%) as white solid. LCMS [M+H]⁺=1175.4

### Step 3: UB-181359

Compound UB-181359d (40 mg, 0.03 mmol) was dissolved in DCM (3 mL), then HCl/dioxane (1 mL) was added, and the mixture was reacted at room temperature for 30 min. The reaction solution was concentrated and then pulped with diethyl ether to obtain target product UB-181359 (20 mg, yield 55%) as white solid. LCMS [M/2+H]⁺=538.4. ¹H NMR (400 MHz, DMSO-d₆,) δ 11.01 (d, J=3.0 Hz, 1H), 9.40 (s, 1H), 9.03 (s, 1H), 8.56 (s, 2H), 8.31 (s, 1H), 8.07 (d, J=8.2 Hz, 1H), 7.93 (d, J=6.4 Hz, 1H), 7.85 (s, 1H), 7.77-7.55 (m, 3H), 7.50 (dd, J=9.2, 5.7 Hz, 2H), 6.62 (d, J=3.8 Hz, 1H), 5.24-5.03 (m, 1H), 4.55-4.27 (m, 3H), 4.28-4.11 (m, 1H), 4.05 (s, 1H), 3.93 (d, J=10.3 Hz, 4H), 3.86-3.74 (m, 1H), 3.71 (t, J=6.6 Hz, 1H), 3.63 (q, J=7.1, 5.2 Hz, 2H), 3.53 (d, J=15.5 Hz, 2H), 3.23 (s, 3H), 3.16 (s, 1H), 2.92 (td, J=13.3, 6.7 Hz, 1H), 2.76 (dt, J=38.4, 6.5 Hz, 2H), 2.59 (d, J=17.7 Hz, 1H), 2.42 (d, J=13.6 Hz, 1H), 2.28-2.12 (m, 1H), 2.09-1.71 (m, 13H), 1.72-1.41 (m, 8H), 1.10-0.86 (m, 12H), 0.76 (td, J=7.4, 4.7 Hz, 3H).

### Synthesis of Compound UB-181312

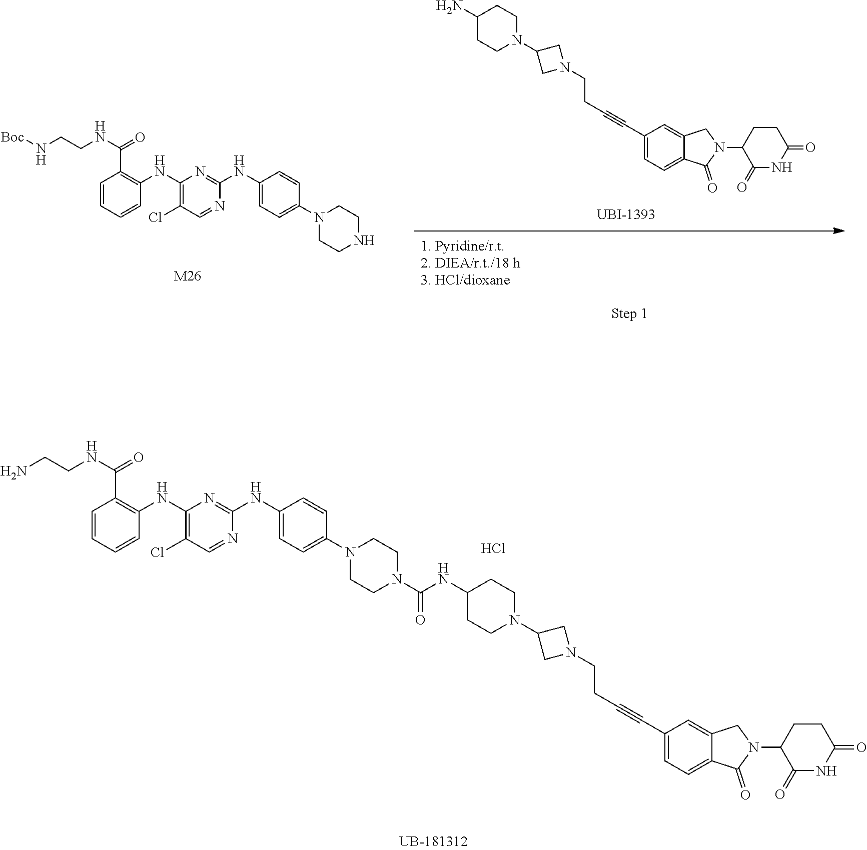 [SMILES: CC(C)(C)OC(=O)NCCNC(=O)c1ccccc1Nc1nc(Nc2ccc(N3CCNCC3)cc2)ncc1Cl ; NC1CCN(C2CN(CCC#Cc3ccc4c(c3)CN(C3CCC(=O)NC3=O)C4=O)C2)CC1 ; "M26" ; NCCNC(=O)c1ccccc1Nc1nc(Nc2ccc(N3CCN(C(=O)NC4CCN(C5CN(CCC#Cc6ccc7c(c6)CN(C6CCC(=O)NC6=O)C7=O)C5)CC4)CC3)cc2)ncc1Cl ; Cl]

### Step 1: UB-181312

General Method 4. LCMS [M+H]⁺+=943.2

### Synthesis of Compound UB-181327

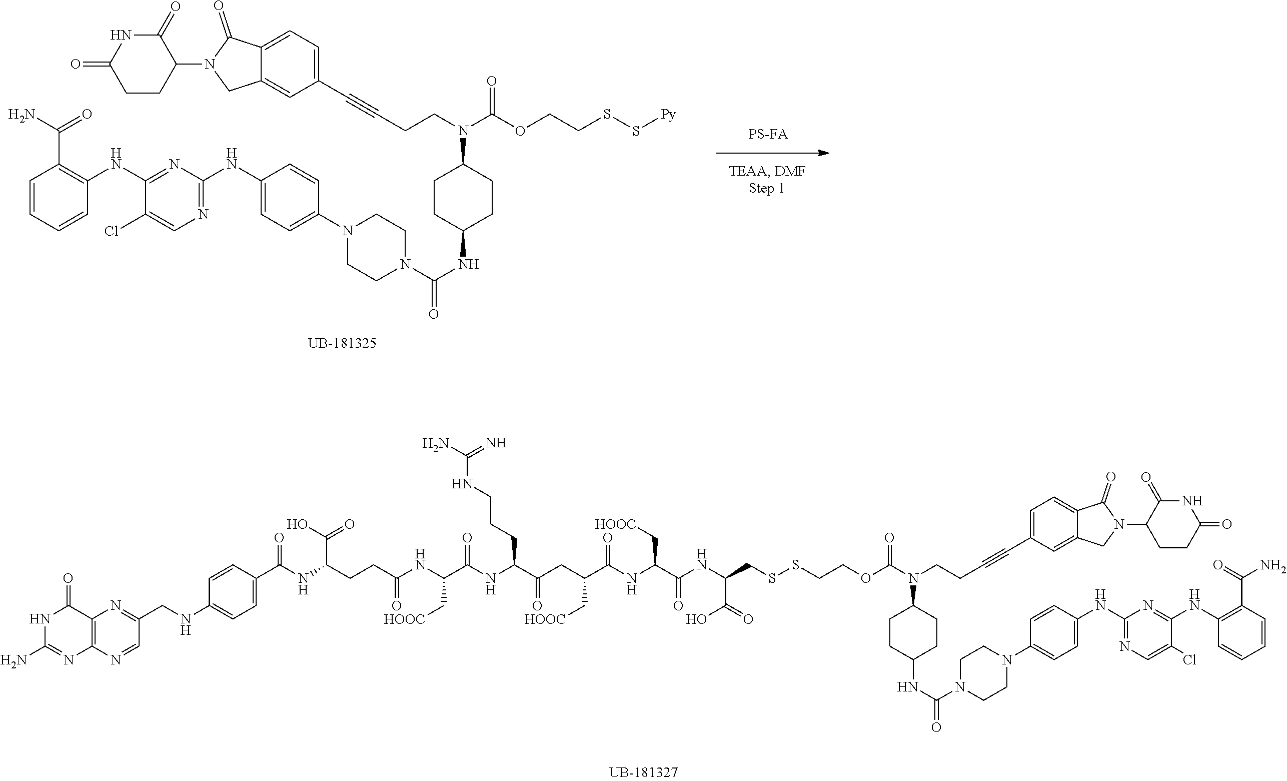

### Step 1: UB-181327

UB-181325 (100 mg, 0.093 mmol) was dissolved in DMF (5 ml), and the mixture was added dropwise to PS-FA (117 mg, 0.112 mmol) and TEAA (2.5 ml) at room temperature. The mixture was reacted for half an hour at room temperature. The reaction solution was purified by C-18 reversed-phase column chromatograph, MeCN/H2O/50 mmol/1 TEAA, and medium pressure preparative chromatography, MeCN/H2O/50 mmol/LNH₄HCO₃, to give product V3441-115 (84.1 mg, 45% yield) as yellow solid. LCMS[M/2+H]=1004.22

### Synthesis of Compound UB-181342

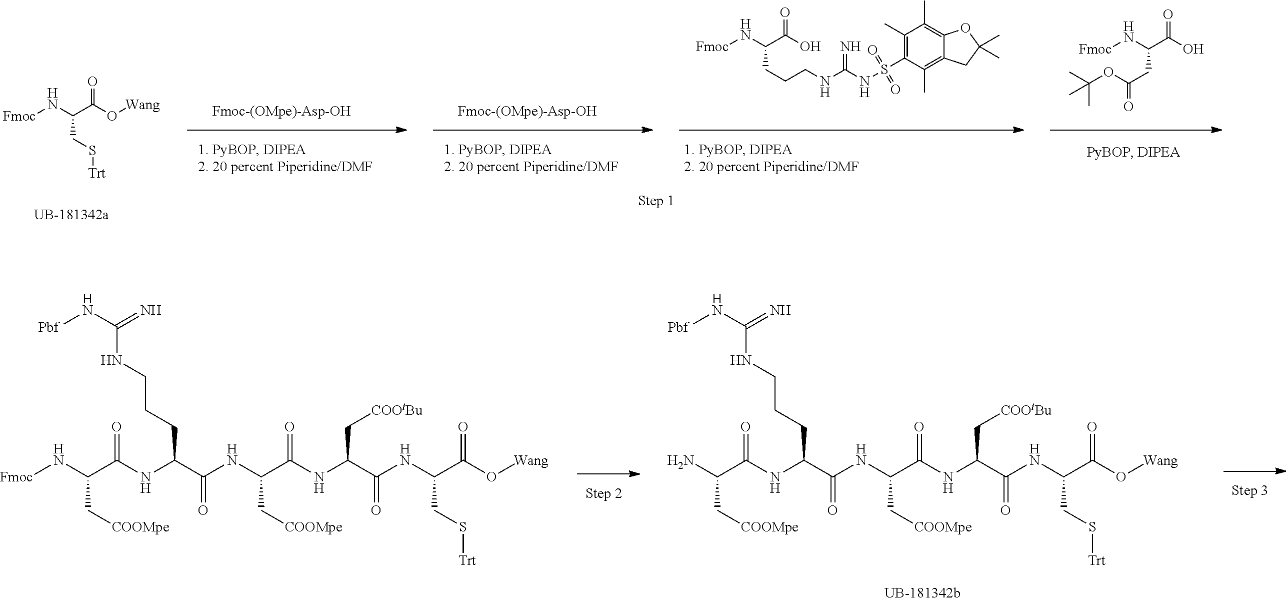

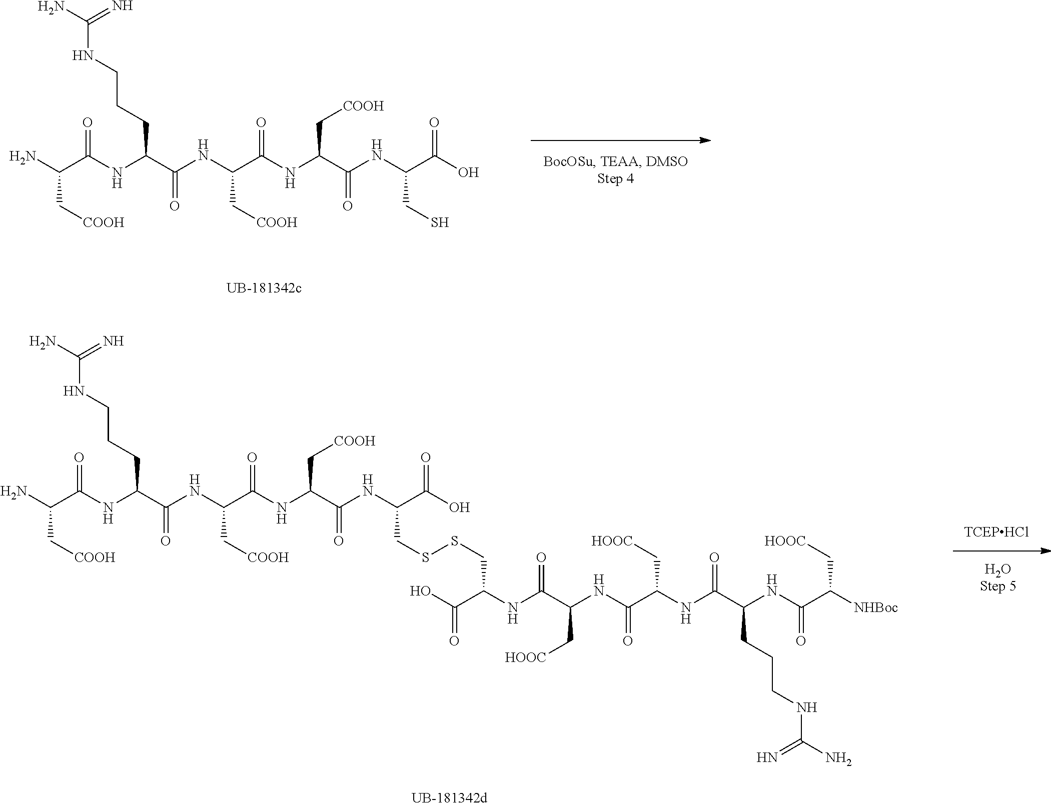 [SMILES: N=C(N)NCCC[C@H](NC(=O)[C@@H](N)CC(=O)O)C(=O)N[C@@H](CC(=O)O)C(=O)N[C@@H](CC(=O)O)C(=O)N[C@@H](CS)C(=O)O ; CC(C)(C)OC(=O)N[C@@H](CC(=O)O)C(=O)N[C@@H](CCCNC(=N)N)C(=O)N[C@@H](CC(=O)O)C(=O)N[C@@H](CC(=O)O)C(=O)N[C@@H](CSSC[C@H](NC(=O)[C@H](CC(=O)O)NC(=O)[C@H](CC(=O)O)NC(=O)[C@H](CCCNC(=N)N)NC(=O)[C@@H](N)CC(=O)O)C(=O)O)C(=O)O]

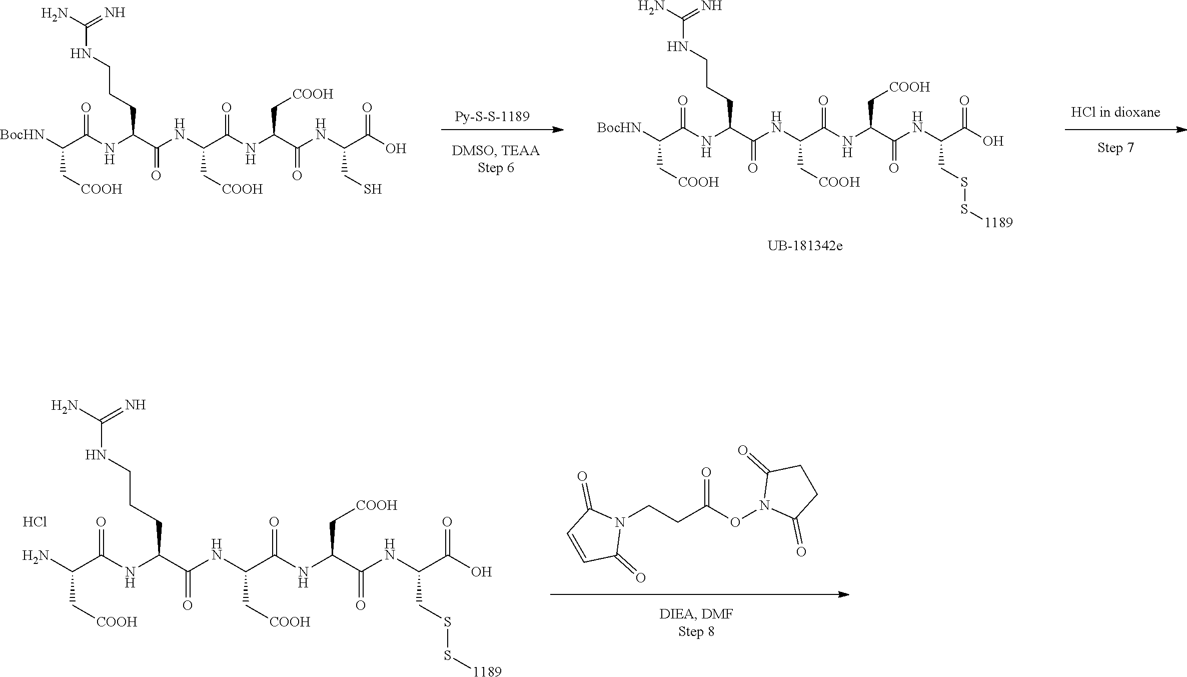

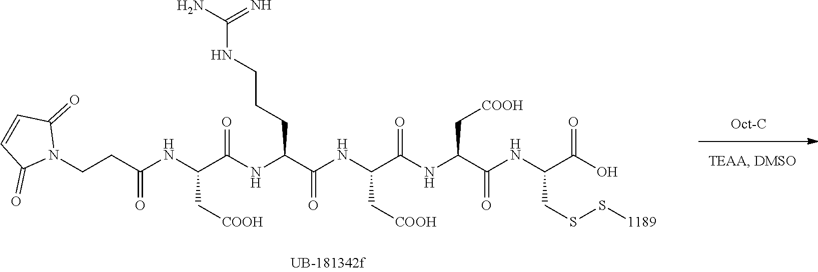

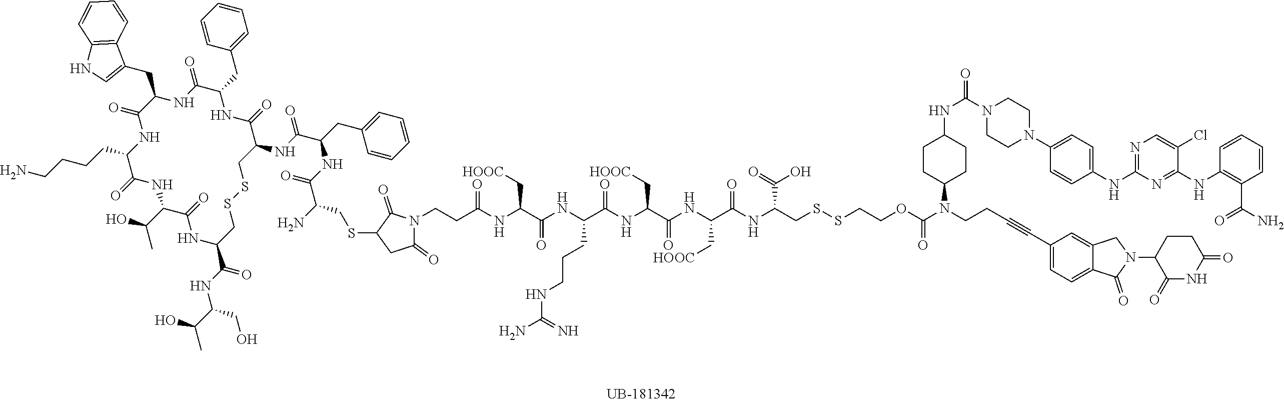

### Step 1: UB-181342b

Compound UB-181342a resin was placed in a reactor with a filter device, added with 50 mL dichloromethane, and reacted for 1 hour on the shaker. The resin was obtained after filtration and was added to the piperidine/DMF (1:4) solution to react for 15 minutes, and then washed with anhydrous DMF (5×30 mL×0.5 min) to obtain the resin.

N-Fluorenylmethoxycarbonyl-L-aspartate-4-tert-butyl ester (3.8 g, 9.2 mmol), PyBOP (7.2 g, 13.8 mmol), and DIPEA (2.4 mL, 6.9 mL) were dissolved in DMF, and then added with Resin. The mixture was reacted for 2h on a shaker. Then the mixture was washed with DMF (5×30 mL×0.5 min). The resulting resin was added to piperidine/DMF (1:4) solution and reacted for 15 minutes, then washed with anhydrous DMF (5×30 mL×0.5 min) to obtain the resin.

N-Fluorenylmethoxycarbonyl-L-aspartate-4-tert-butyl ester (3.8 g, 9.2 mmol), PyBOP (7.2 g, 13.8 mmol), and DIPEA (2.4 mL, 6.9 mL) were dissolved in DMF, and then added with Resin. The mixture was reacted for 2h on a shaker. Then the mixture was washed with DMF (5×30 mL×0.5 min). The resulting resin was added to piperidine/DMF (1:4) solution and reacted for 15 minutes, then washed with anhydrous DMF (5×30 mL×0.5 min) to obtain the resin.

N-Fluorenylmethoxycarbonyl-Pbf-L-arginine (6 g, 9.2 mmol), PyBOP (7.2 g, 13.8 mmol), and DIPEA (2.4 mL, 6.9 mL) were dissolved in DMF, and then added with Resin. The mixture was reacted for 2h on a shaker. Then the mixture was washed with DMF (5×30 mL×0.5 min). The resulting resin was added to piperidine/DMF (1:4) solution and reacted for 15 minutes, then washed with anhydrous DMF (5×30 mL×0.5 min) to obtain the resin.

N-Fluorenylmethoxycarbonyl-L-aspartate-4-tert-butyl ester (3.8 g, 9.2 mmol), PyBOP (7.2 g, 13.8 mmol), and DIPEA (2.4 mL, 6.9 mL) were dissolved in DMF, and then added with Resin. The mixture was reacted for 2h on a shaker. Then the mixture was washed with DMF (5×30 mL×0.5 min). The resulting resin was added to piperidine/DMF (1:4) solution and reacted for 15 minutes, then washed with anhydrous DMF (5×30 mL×0.5 min) to obtain the resin.

N-(9-Fluorenylmethoxycarbonyl)-L-glutamate-1-tert-butyl ester (3.8 g, 9.2 mmol), PyBOP (7.2 g, 13.8 mmol), and DIPEA (2.4 mL, 6.9 mL) were dissolved in DMF, and then added with resin. The mixture was reacted for 2h on a shaker. Then the mixture was washed with DMF (5×30 mL×0.5 min). The resulting resin was added to piperidine/DMF (1:4) solution and reacted for 15 minutes, then washed with anhydrous DMF (5×30 mL×0.5 min) to obtain the resin.

### Step 2: UB-181342c

The resin was added to TFA/H2O/TIPS/EDT=92.5:2.5:2.5:2.5 solution (100 mL) and the mixture was stirred for 1.5 h. The mixture was filtered to obtain filtrate, and the filtrate was concentrated at low temperature to remove the most of TFA, and added with cold isopropyl ether (100 mL). The isopropyl ether layer was then carefully poured out. The isopropyl ether washing step was repeated for 3 times. Then 80 mL of water was added. The aqueous phase was separated by reversed-phase column chromatograph (CH₃CN/2% TFA in water)=0%˜14% for 15 min, collected at 14%), to obtain compound UB-181342c (420 mg) LCMS [M+H]⁺=623

### Step 3: UB-181342d

Compound UB-181342c (1 g, 1.6 mmol) and BocOSu (1.73 g, 8 mmol) were dissolved in DMSO (6 ml), and the mixture was added with TEAA (3 ml) and stirred at room temperature overnight. The reaction solution was separated and purified by reversed-phase chromatograph, CH₃CN/H2O, to give V3927-042 (UB-181342d, 277 mg, 24% yield) as white solid. LCMS[M/2+H]=723.2. ¹H NMR (400 MHz, DMSO) δ 12.65 (s, 2H), 9.30 (d, J=37.2 Hz, 1H), 8.61-8.25 (m, 2H), 7.73 (s, 1H), 7.65-7.50 (m, 1H), 7.22-7.00 (m, 2H), 4.58 (d, J=6.7 Hz, 1H), 4.42 (s, 1H), 4.34 (s, 1H), 4.23 (d, J=5.1 Hz, 2H), 3.31-2.85 (m, 5H), 2.61 (dt, J=25.9, 5.3 Hz, 4H), 1.85 (s, 1H), 1.69-1.42 (m, 3H), 1.38 (s, 9H).

### Step 4: UB-181342e

Compound UB-181342d (100 mg, 0.069 mmol) and TCEP·HCl (20 mg, 0.069 mmol) were dissolved in H₂O (2 ml) and the mixture was stirred at 40° C. for 1 hour. A solution of Compound Py-S—S-1189 (162 mg, 0.152 mmol) dissolved in DMSO (6 ml) and TEAA (1 ml) was added to the reaction solution and the mixture was stirred at 40° C. for 1 hour. The reaction solution was separated and purified by reversed-phase chromatograph, CH₃CN/H2O, to give V3927-046 (UB-181342e, 80 mg, 69% yield) as yellow solid. LCMS[M/2+H]=842.8

### Step 5: UB-181342f

Compound UB-181342e (70 mg, 0.042 mmol) was dissolved in HCl in dioxane (2 mL), and the mixture was stirred at room temperature for 1 hour. The reaction solution was spin-dried and dissolved in DMF (1 ml), and added with compound MP (33 mg, 0.125 mmol) and DIEA (54 mg, 0.42 mmol). The mixture was stirred at 40° C. for 1 h. The reaction solution was separated and purified by reversed-phase chromatograph, CH₃CN/H2O, to give V3927-051 (UB-181342f, 56 mg, 77% yield) as pink solid. LCMS[M/2+H]=868.3

### Step 6: UB-181342

Compound UB-181342f (56 mg, 0.0323 mmol) and Oct-C (72 mg, 0.0646 mmol) were dissolved in DMSO (1 ml) and the above reaction solution was added with TEAA (0.5 ml) and stirred at 30° C. for 1 hour. The reaction solution was separated and purified by reversed-phase chromatograph, MeCN/H2O/50 mmol/l TEAA, to give V3927-054 (UB-181342, 3.9 mg, 4.2% yield) as white solid. LCMS[M/3+H]=953.81

### Synthesis of Compound UB-181343

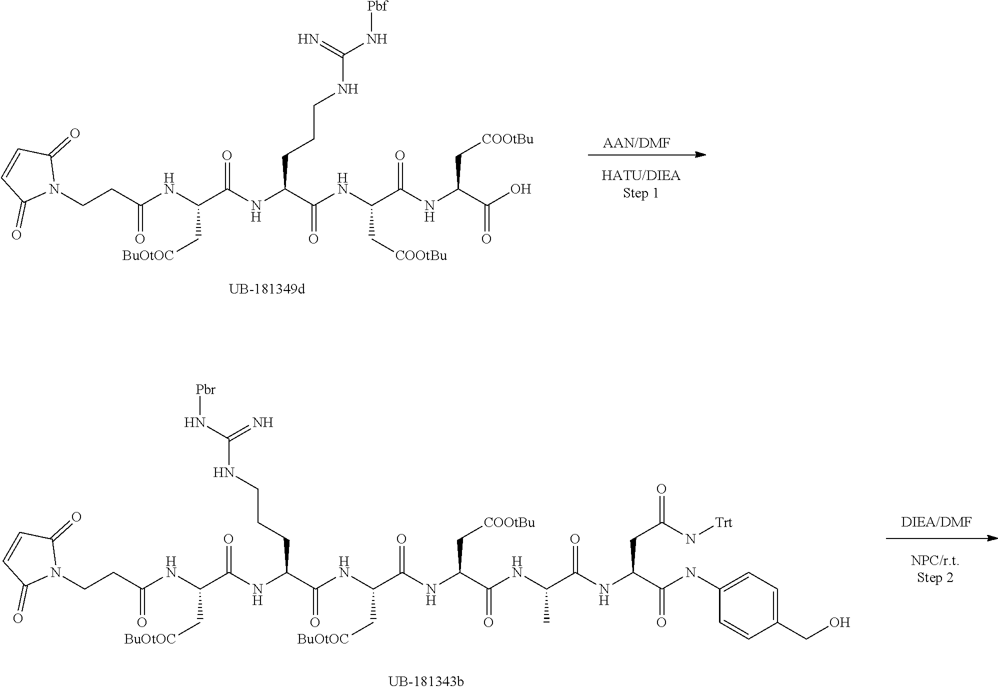

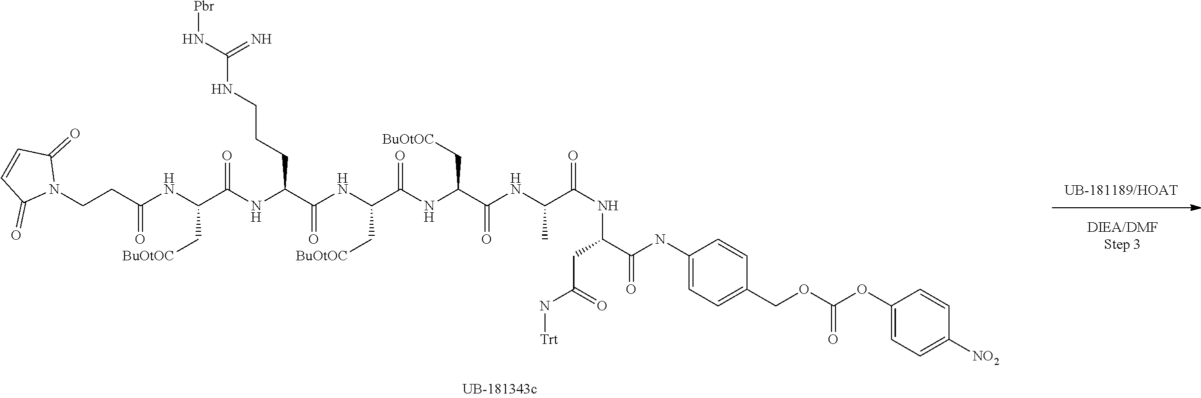

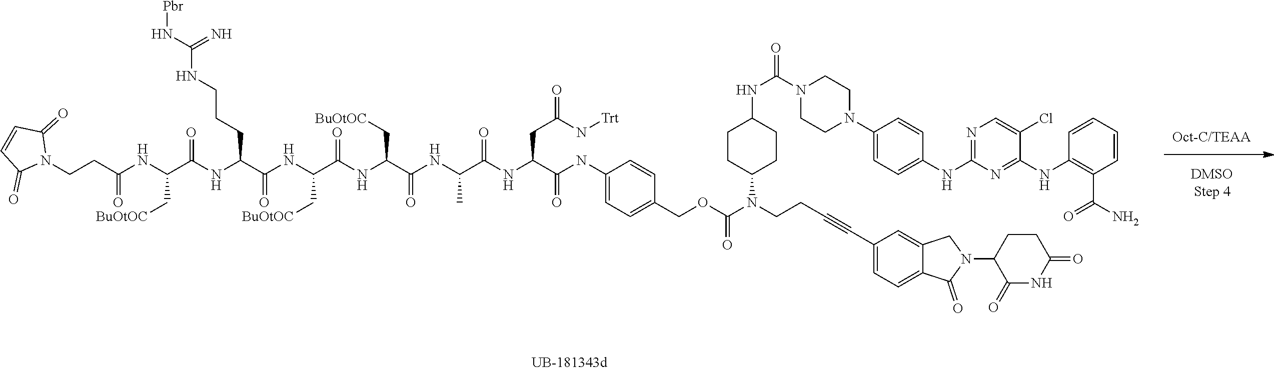

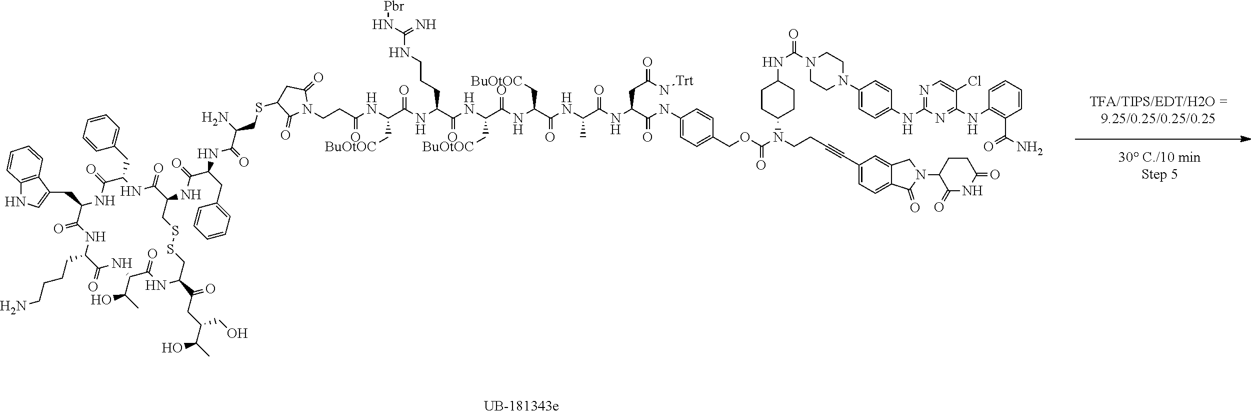

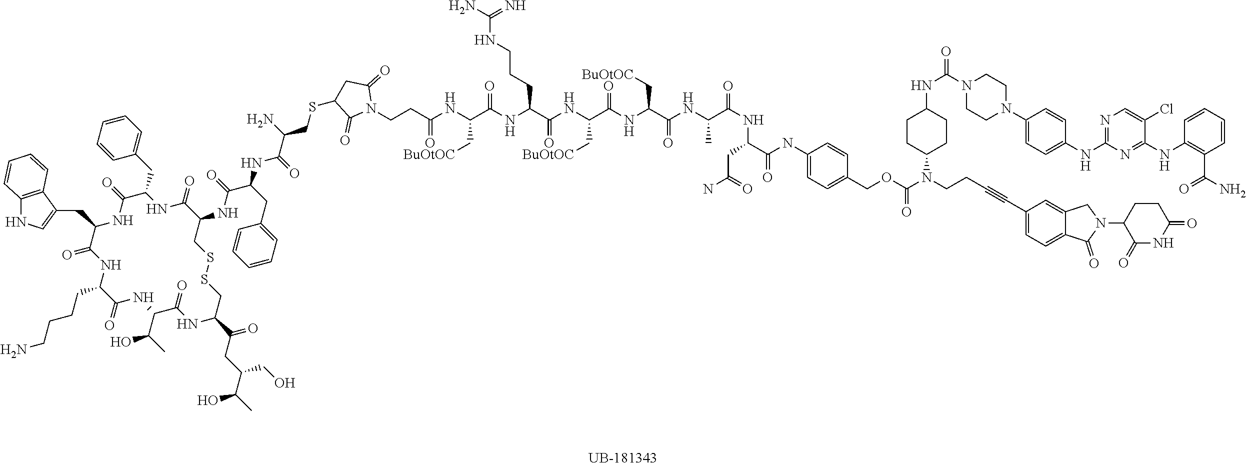

### Step 1: UB-181343b

To a solution of UB-181349d (500 mg, 0.5 mmol), HATU (261.5 mg, 0.7 mmol) and DIEA (118.3 mg, 0.9 mmol) in DMF (5 ml) was added UB-181149g (252.3 mg, 0.5 mmol). The reaction solution was reacted at room temperature for 2 h. The reaction solution was purified by reversed-phase column to give UB-181343b (334 mg, 45% yield) as white solid. LCMS=[M+3H]+=1625.10.

### Step 2: UB-181343c

To a solution of UB-181343b (2.2 g, 1.35 mmol) and NPC (824 mg, 4.07 mmol) in DMF (20 ml) was added DIEA (350 mg, 4.07 mmol). The reaction solution was stirred at 30° C. for 2 h.

The reaction solution was spin-dried in vacuum, and the resulting mixture was purified by normal phase column (DCM/MeOH=0 to 10%) to give UB-181343c (1.5 g, 70% yield) as yellow solid. LC-MS: [M+3H]+=1790.3.

### Step 3: UB-181343d

To a solution of UB-181189 (330 mg, 0.38 mmol) and HOAT (52 mg, 0.38 mmol) in DMF (20 ml) was added UB-181343c (680 mg, 0.38 mmol) and DIEA (147 mg, 1.14 mmol). The reaction solution was stirred at 30° C. for 4 h. The reaction solution was spin-dried in vacuum, and the resulting mixture was purified by normal phase column (DCM/MeOH:THF=1:1=0˜30%) to give UB-181343d (430 mg, 40% yield) as yellow solid. LC-MS: [1/2M+3H]+=1255.6.

### Step 4: UB-181343e

To a solution of UB-181343d (240 mg, 0.1 mmol) and Oct-C (214 mg, 0.2 mmol) in DMSO (4 ml) was added TEAA (2 ml). The reaction solution was stirred at 30° C. for 4 h. The reaction solution was purified by reversed-phase column (H2O (TEAA50 mmol): acetonitrile=0-100%) to afford UB-181343e (340 mg, 97% yield) as white solid. LC-MS: [1/3M+3H]+=1212.2.

### Step 5: UB-181343

UB-181343e (300 mg, 0.08 mmol) was dissolved in TFA/TIPS/H2O/EDT-82.5/2.5/2.5/2.5 (8 ml), and the mixture was stirred at 30° C. for 15 min. The reaction solution was added with diethyl ether and continued to stir at 30° C. for 1 h. The solid obtained by filtering the reaction solution was purified by reversed-phase column chromatograph (H2O:CH₃CN=0-100%) to afford UB-181343 (40 mg, 16% yield) as white solid. LC-MS: [1/3M+3H]+=991.1.

### Synthesis of Compound UB-181344

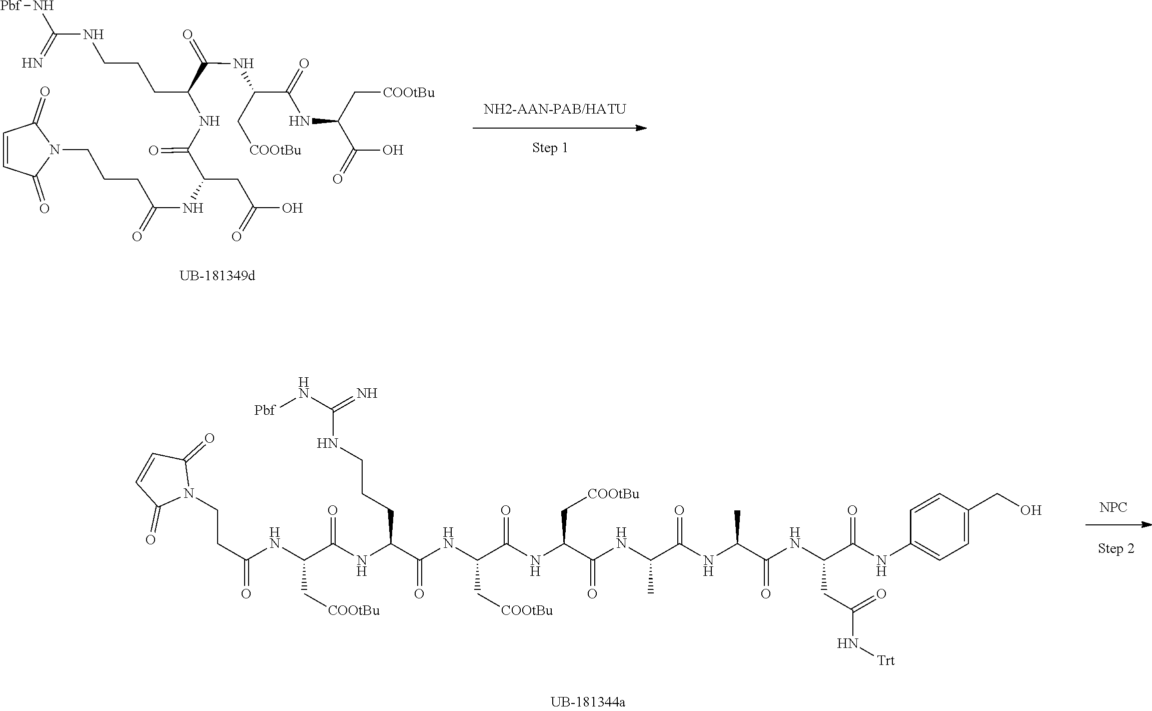

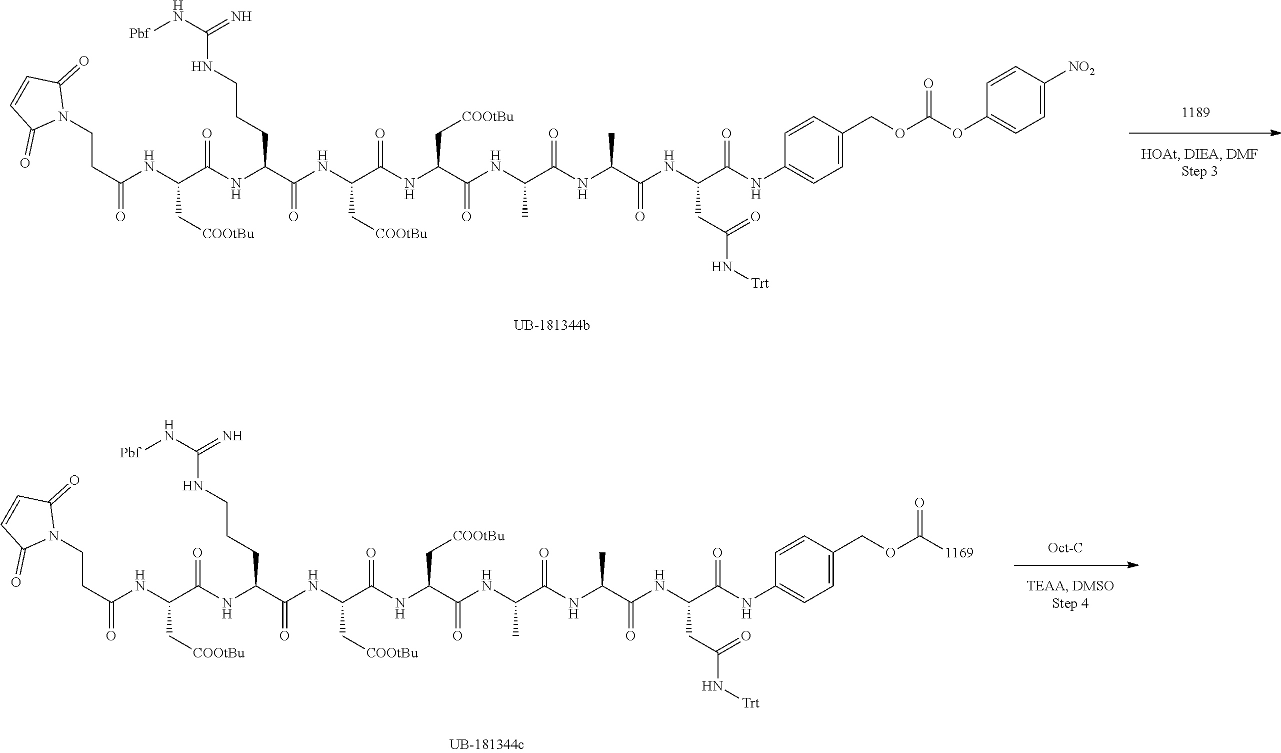

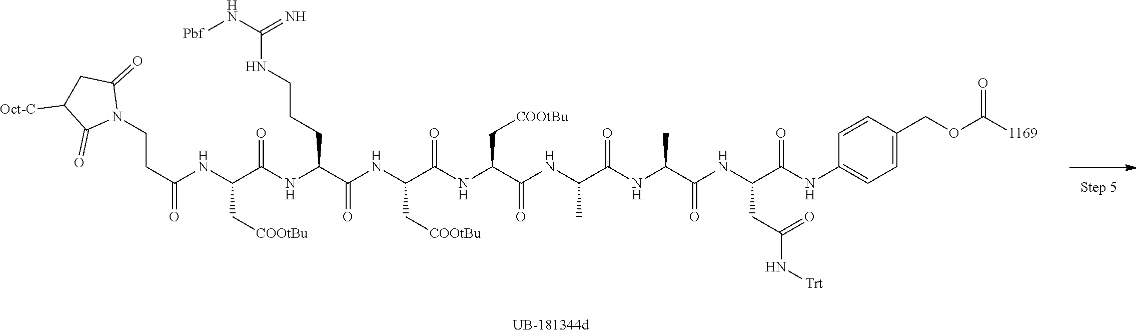

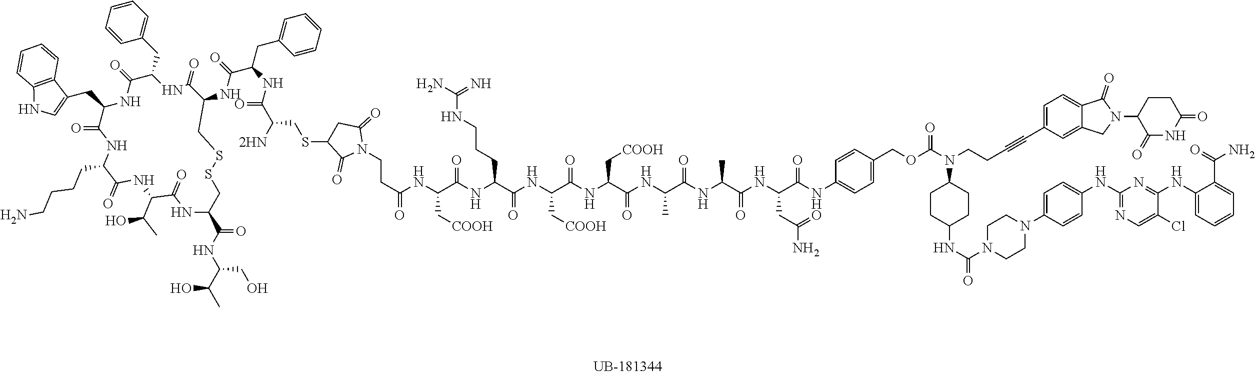

### Step 1: UB-181344a

Compound UB-181349d (109 mg, 0.1 mmol) was dissolved in DMF (1 mL), and added with HATU (76 mg, 0.2 mmol), DIEA (25.8 mg, 0.2 mmol) and NH2-AAN-PAB (63.1 mg, 0.1 mmol). The mixture was stirred at room temperature for 1 hour. The reaction solution was purified by silica gel chromatography (DCM/MeOH=0˜50%) to obtain product UB-181344a (85 mg, 50% yield) as white solid. LCMS [M+1]+=1697.0

### Step 2: UB-181344b

Compound UB-181344a (3000 mg, 1.77 mmol) was dissolved in DMF (20 mL), and added with DIEA (456 mg, 3.54 mmol) and NPC (1070 mg, 3.54 mmol). The mixture was stirred at room temperature for 2 hours. The mixture was purified by silica gel chromatography (DCM/MeOH=0˜50%) to obtain UB-181344b (1800 mg, 55% yield) as white solid. LCMS [M+1]+=1861.0

### Step 3: UB-181344c

Compound UB-181344b (1300 mg, 0.7 mmol) and DIEA (181 mg, 1.40 mmol) were added with compound 1189 (599 mg, 0.7 mmol), HOAt (190 mg, 1.40 mmol), and dissolved in DMF (5 ml). The mixture was stirred at 30° C. for 4 hours. The mixture was spin-dried and purified by silica gel chromatography DCM/MeOH:THF=1:10-30% to obtain UB-181344c (1080 mg, 60% yield) as yellow solid. LCMS [M/2+H]=1291.0

### Step 4: UB-181344d

TEAA (1 mL) was added with compound UB-181344c (200 mg, 0.078 mmol), Oct-C (87 mg, 0.078 mmol), and dissolved in DMSO (2 ml). The mixture was stirred at 30° C. for 0.5 hour. The mixture was spin-dried and purified by reverse phase chromatography MeCN/H2O/50 mmol/l TEAA to obtain UB-181344d (UB-181349e, 100 mg, 35% yield) as white solid. LCMS [M/3+H]=1235.8

### Step 5: UB-181344

UB-181344d (300 mg, 0.08 mmol) was dissolved in TFA/TIPS/H2O/EDT-82.5/2.5/2.5/2.5 (8 ml) and stirred at 30° C. for 15 min. The reaction solution was added with diethyl ether and continued to stir at 30° C. for 1h. The solid obtained by filtering the reaction solution was purified by a reversed-phase column chromatograph (H2O:CH₃CN=0-100%) to afford UB-181344 (31.5 mg, 20% yield) as white solid. LC-MS: [1/3M+3H]+=1015.0.

### Synthesis of Compound UB-181345

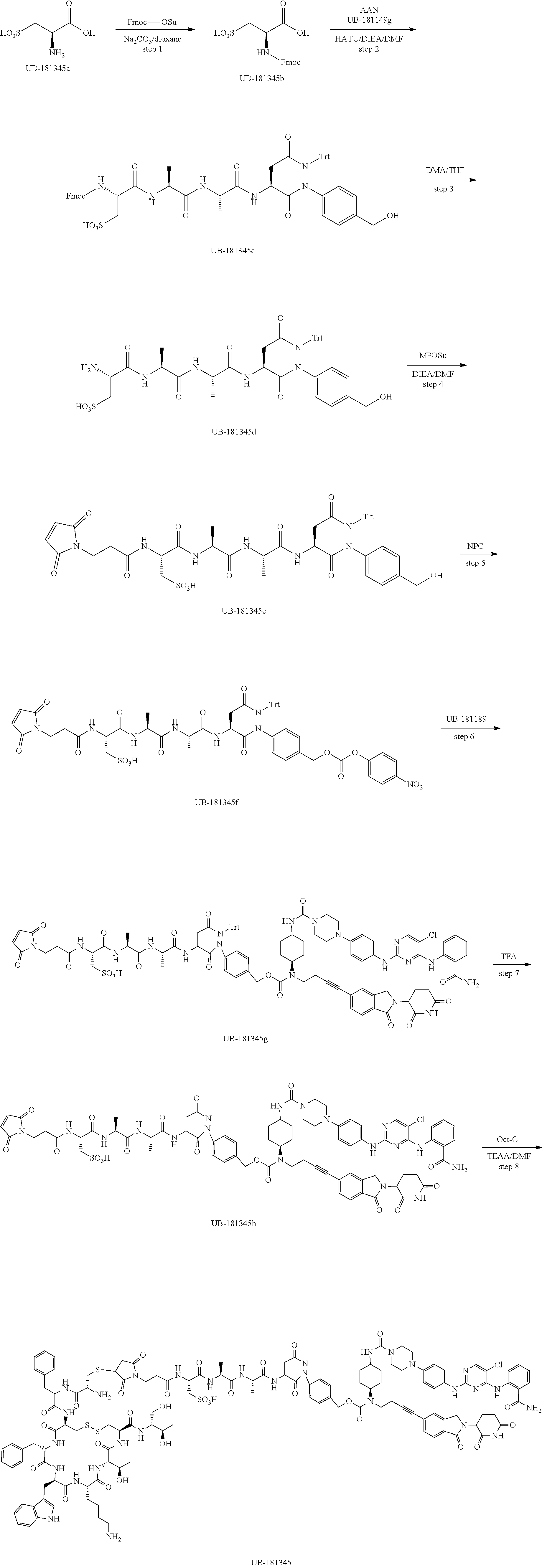

### Step 1: UB-181345b

Compound UB-181345a (1 g, 6 mmol) was dissolved in dioxane (25 mL), then added with 10% Na₂CO₃ (35 mL), and slowly added dropwise with Fmoc-OSu (2.4 g, 7.1 mmol) dissolved in dioxane (15 mL) under ice water bath. Then the mixture was reacted at room temperature for 16 hours. The reaction solution was spun to remove dioxane, the residue was diluted with water, and washed once with methyl tert-butyl ether, and the aqueous phase was lyophilized after being adjusted to pH=3 with 3N HCl. The solid was pulped with MeCN/H₂O=4/1 to obtain crude product UB-181345b (1.5 g, yield 65%) as white solid. LCMS [M−H]⁺=390.9

### Step 2: UB-181345c

Compound UB-181345b (330 mg, 0.84 mmol) was dissolved in DMF (3 mL), and then HATU (417 mg, 1.10 mmol) and DIEA (327 mg, 2.53 mmol) were added. 10 min later, UB-181149g (420 mg, 0.68 mmol) was added and the mixture was reacted at room temperature for 2 hours. The reaction solution was concentrated and then separated by reversed phase column chromatography to obtain target product UB-181345c (200 mg, yield 24%) as white solid. LCMS [M−H]⁺=994.3

### Step 3: UB-181345d

Compound UB-181345c (200 mg, 0.20 mmol) was dissolved in THF (3 mL), and then added with dimethylamine in tetrahydrofuran (6 mL), and the mixture was reacted at room temperature for 2 hours. The reaction solution was concentrated and then the residue was washed with diethyl ether to obtain target product UB-181345d (150 mg, yield 97%) as white solid. LCMS [M−H]⁺=772.0

### Step 4: UB-181345e

Compound UB-181345d (179 mg, 0.23 mmol) was dissolved in DMF (3 mL), and then added with DIEA (81 mg, 0.63 mmol). After reacting at room temperature for 1 hour, the mixture was added with MpOSu (56 mg, 0.21 mmol) and stirred for another 2 hours. The reaction solution was concentrated and then separated by column chromatography (MeOH/DCM=20%) to give UB-181345e (90 mg, 42% yield) as white solid. LCMS [M−H]⁺=923.2

### Step 5: UB-181345f

Compound UB-181345e (200 mg, 0.29 mmol) was dissolved in DMF (2 mL), and then DIEA (114 mg, 0.88 mmol) and NPC (134 mg, 0.44 mmol) were added. The reaction solution was reacted at room temperature for 2 hours. The reaction solution was separated by reversed phase column chromatography to obtain target product UB-181345f (150 mg, yield 64%) as yellow solid. LCMS [M+H]⁺=1090.5

### Step 6: UB-181345g

Compound UB-181345f (220 mg, 0.20 mmol) was dissolved in DMF (2 mL), and then UB-181189 (86 mg, 0.10 mmol), HOBT (55 mg, 0.40 mmol) and DIEA (78 mg, 0.61 mmol) were added. The mixture was reacted at room temperature for 16 hours. The reaction solution was concentrated and then separated by column chromatography (MeOH/DCM=1/10) to give target product UB-181345g (100 mg, 55% yield) as yellow solid. LCMS [M+H]⁺=1807.8

### Step 7: UB-181345h

Compound UB-181345g (30 mg, 0.02 mmol) was dissolved in DCM/TFA (3.5/1.5 mL), and then a catalytic amount of iPr₂SiH was added, and the mixture was reacted at room temperature for 10 min. The reaction solution was concentrated at low temperature and then the residue was pulped with diethyl ether to obtain target product UB-181345h (20 mg, yield 77%) as yellow solid. LCMS [M−H]⁺=1564.5

### Step 8: UB-181345

Compound UB-181241d (43 mg, 0.04 mmol) was dissolved in TEAA buffer solution (1 mL), and then added with UB-181345h (30 mg, 0.02 mmol) in DMF (2 mL). The reaction solution was reacted at room temperature for 2 hours, and then subjected to reverse phase column chromatography (MeCN/NaH₂PO₄ buffer) to afford 300 mg white solid (containing salt), which was then subjected to preparative chromatography to afford target product UB-181345 (17 mg, yield 33%) as white solid. LCMS [M/2-H]⁺=1344.5

### Synthesis of Compound UB-181347

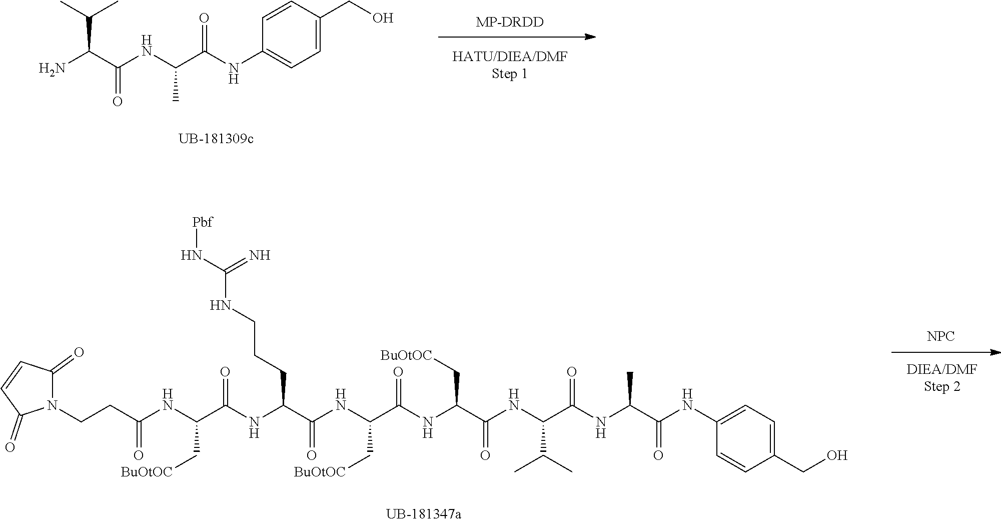

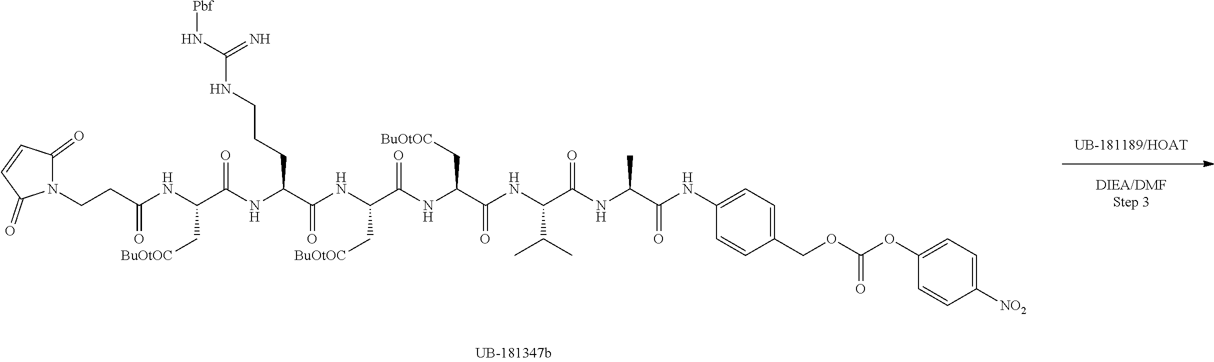

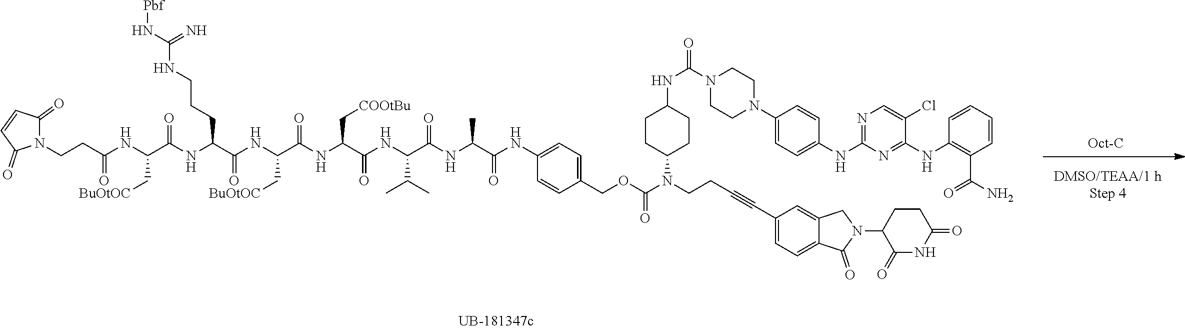

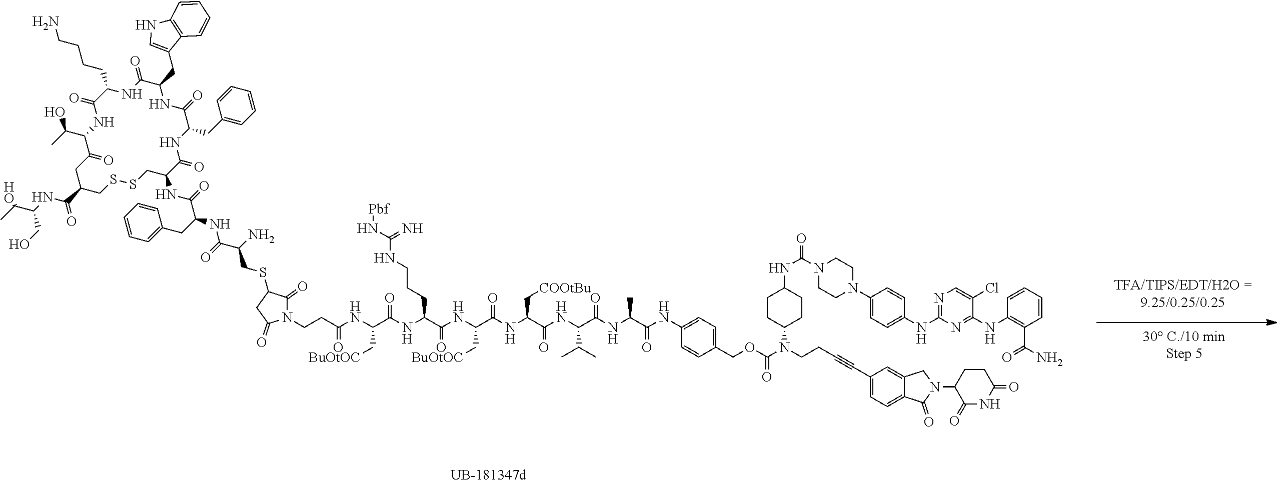

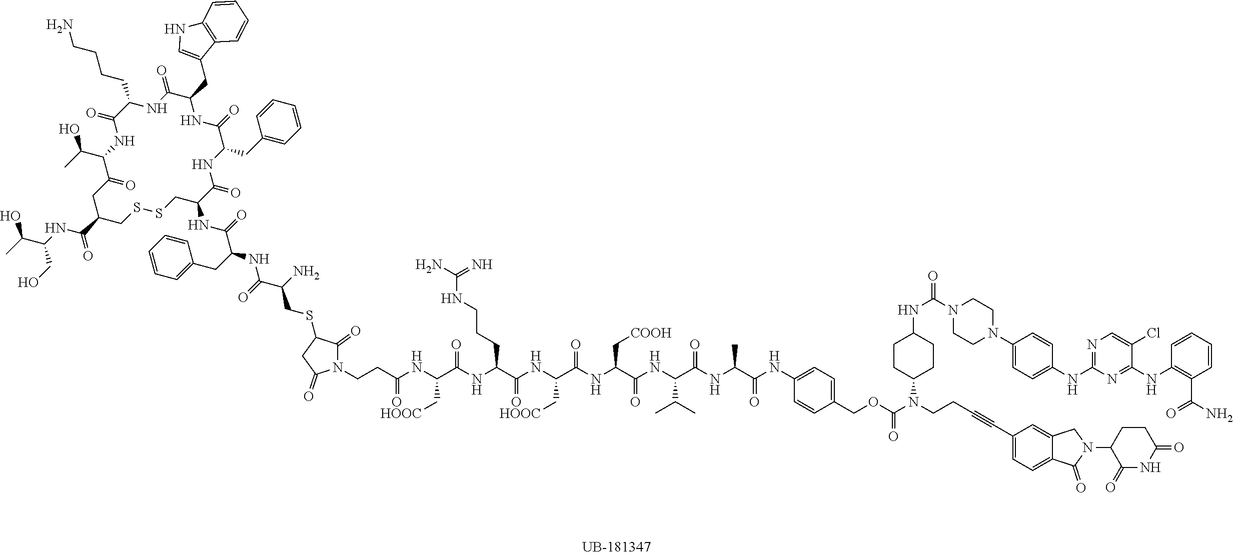

### Step 1: UB-181347a

To a solution of UB-181309c (53.8 mg, 0.2 mmol), MP-DRDD (200 mg, 0.2 mmol) and HATU (104.6 mg, 0.3 mmol) in DMF (2 ml) was added DIEA (47.3 mg, 0.4 mmol). The reaction solution was stirred at room temperature for 2 h. The reaction solution was purified by reversed-phase column chromatography (H₂O:Acetonitrile=0-100%) to afford UB-181347a (146 mg, 58% yield) as white solid. LC-MS: [M+H]+=1367.9.

### Step 2: UB-181347b

To a solution of UB-181347a (500 mg, 0.37 mmol) and NPC (223 mg, 0.73 mmol) in DMF (10 ml) was added DIEA (95 mg, 0.73 mmol). The reaction solution was stirred at 30° C. for 2 h. The reaction solution was spin-dried, and the resulting mixture was purified by normal phase column (DCM/MeOH:THF=1:1=0-20%) to give UB-181347b (520 mg, 80% yield) as yellow solid. LC-MS: [M+2H]⁺=1533.0.

### Step 3: UB-181347c

To a solution of UB-181189 (616 mg, 0.7 mmol) and DIEA (280 mg, 2.1 mmol) in DMF (10 ml) was added UB-181347b (1.1 g, 0.7 mmol) and HOAT (98 mg, 0.7 mmol). The reaction solution was stirred at 30° C. for 4 h. The reaction solution was spin-dried in vacuum, and the resulting mixture was purified by normal phase column chromatography (DCM/MeOH:THF=1:1=0-30%) to give UB-181347c (900 mg, 60% yield) as yellow solid. LC-MS: [1/2M+3H]⁺=1127.2.

### Step 4: UB-181347d

To a solution of UB-181347c (900 mg, 0.4 mmol) and Oct-C (450 mg, 0.4 mmol) in DMSO (10 ml) was added TEAA (4 ml). The reaction solution was stirred at 30° C. for 2 h. The reaction solution was purified by reversed-phase column chromatography (H2O (TEAA50 mmol): Acetonitrile=0-100%) to afford UB-181347d (1.1 g, 80% yield) as white solid. LC-MS: [1/3M+3H]⁺=1126.2.

### Step 5: UB-181347

UB-181347d (200 mg, 0.06 mmol) was dissolved in TFA/TIPS/H₂O/EDT-92.5/2.5/2.5/2.5 (10 ml). The reaction solution was stirred at 30° C. for 10 MIN. The reaction solution was added with diethyl ether and continued to stir at 30° C. for 1 h. The solid obtained by filtering the reaction solution was subjected to reversed-phase column chromatography (H₂O: Acetonitrile=0-100%) to afford UB-181347 (40 mg, 30% yield) as white solid. LC-MS: [1/3M+3H]+=985.9.

### Synthesis of Compound UB-181348

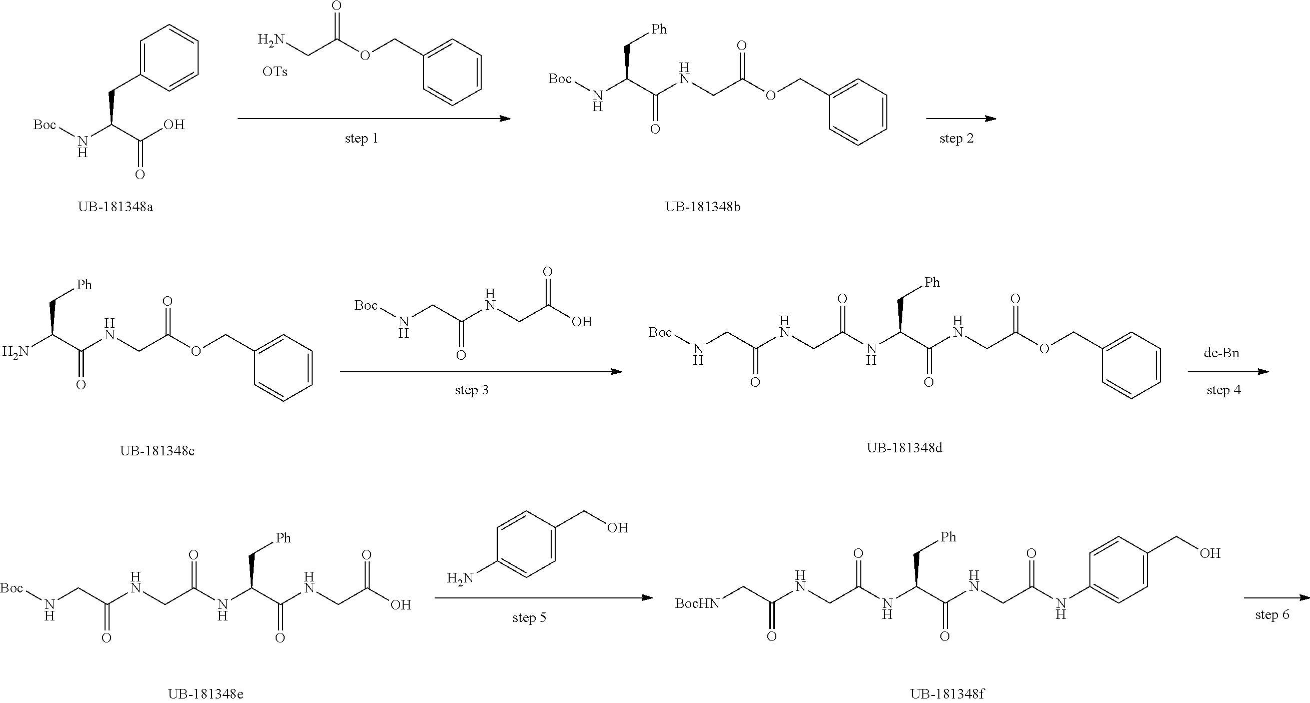

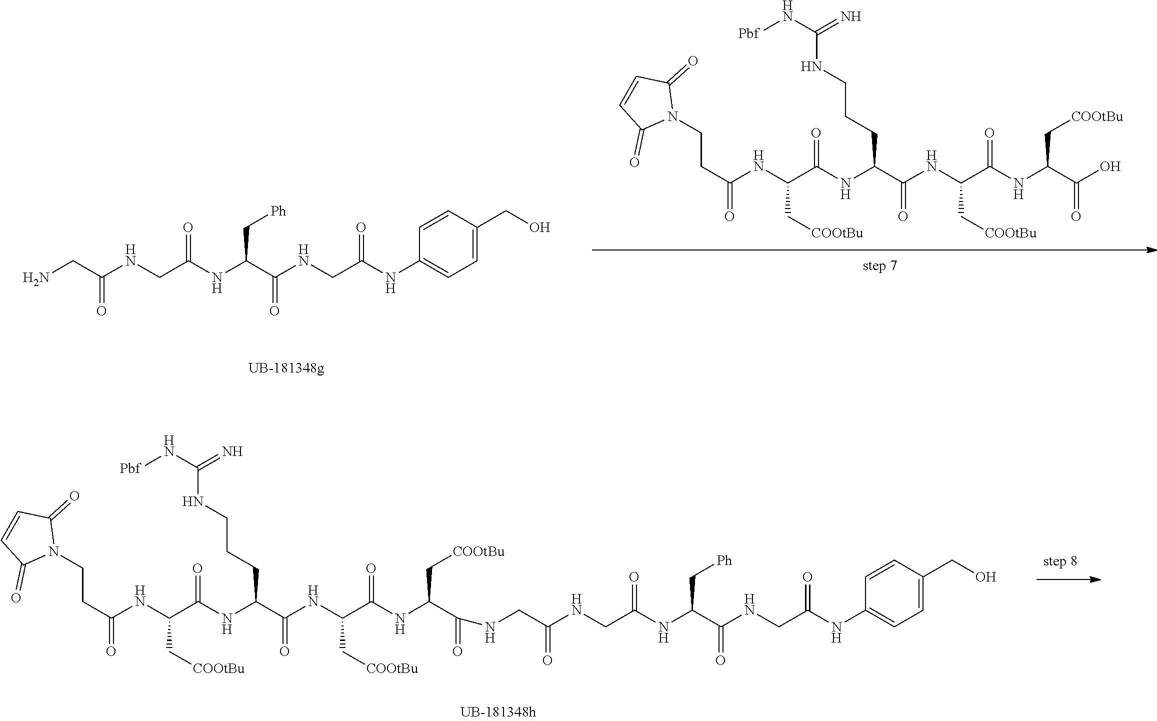

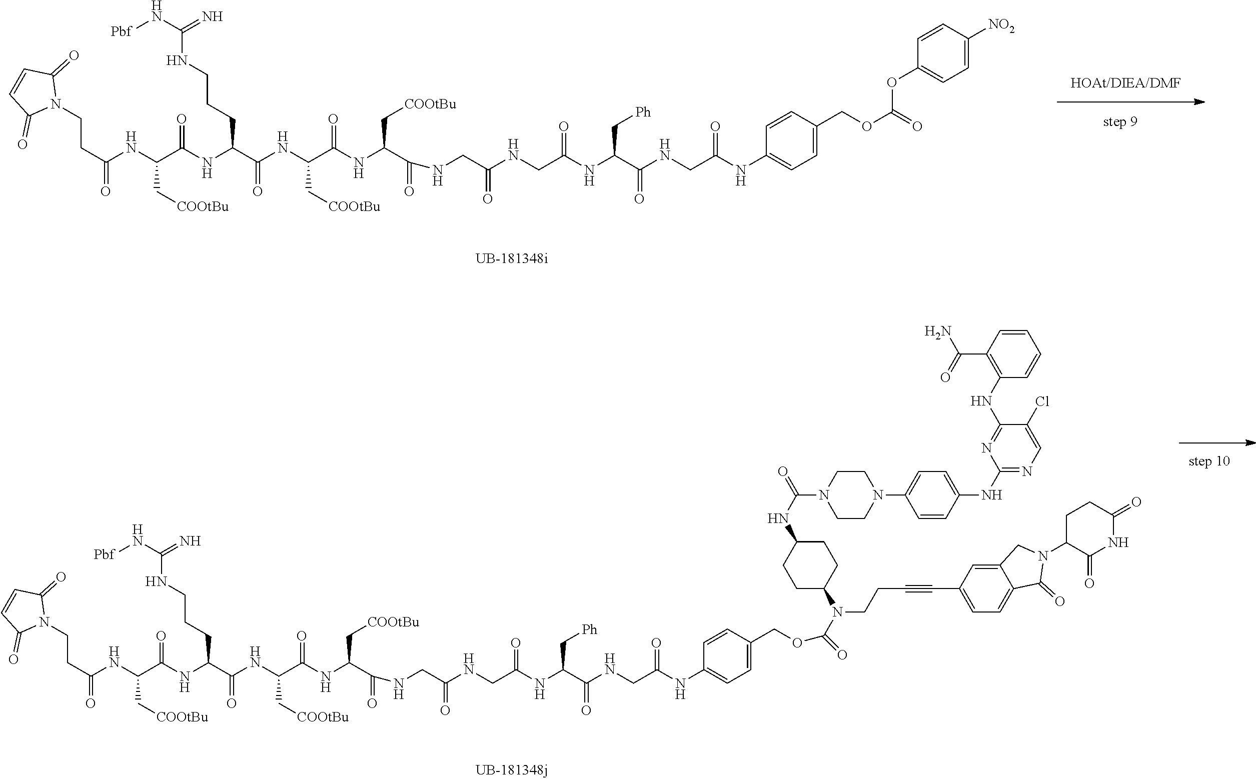

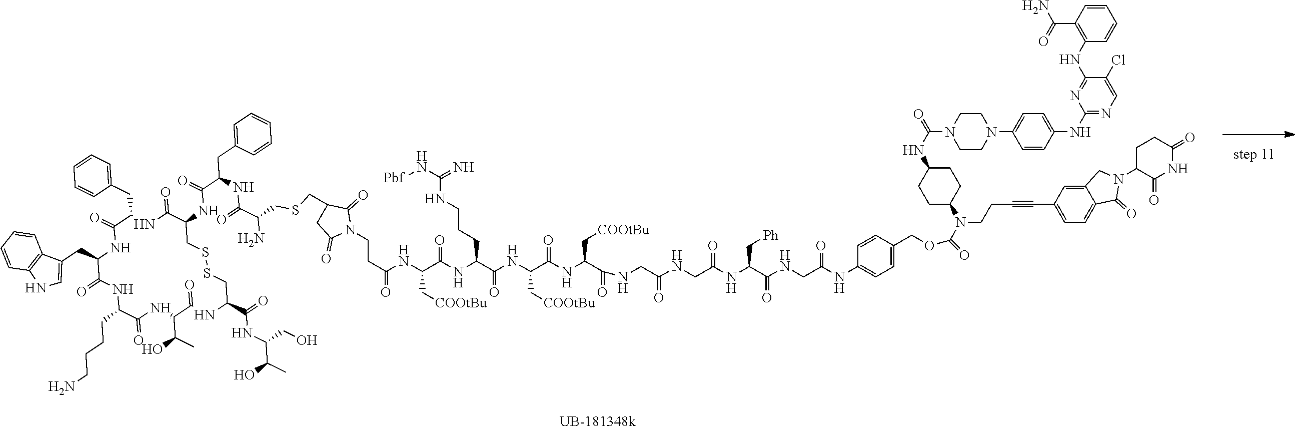

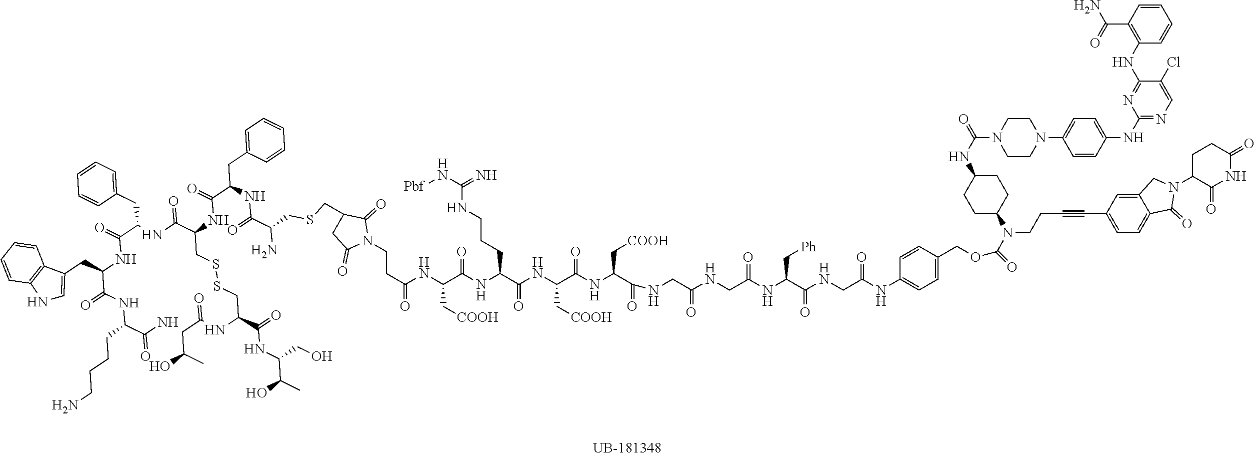

### Step 1: UB-181348b

UB-181348a (5.0 g, 18.9 mmol), Benzyl glycinate (3.1 g, 18.9 mmol), HATU (9.3 g, 24.4 mmol) and DIEA (3.2 g, 24.4 mmol) were dissolved in DMF (50 mL) and the mixture was stirred for 2 hours at room temperature. After completion of the reaction, the mixture was concentrated and purified by silica gel chromatography with DCM/(CH₃₀H:THF=1:1)=10%-40% for 20 min to give compound UB-181348b (7.6 g, 97% yield) as white solid. LCMS:[M+1]+=413.

### Step 2: UB-181348c

To a solution of UB-181348b (3.8 g, 9.2 mmol) in tetrahydrofuran (40 mL) was added dropwise hydrochloric acid in dioxane (25 mL, 4N). The mixture was stirred at room temperature for 2 h. The solvent was removed by rotary evaporation and the crude product was washed with dry isopropyl ether (30 mL*3) to give UB-181348c (2.9 g) as white solid. LCMS:[M+1]⁺=313.

### Step 3: UB-181348d

UB-181348c (2 g, 6.4 mmol), N-(tert-butoxycarbonyl)glycine (1.5 g, 6.4 mmol), HATU (4.6 g, 12.0 mmol) and DIEA (2.31 g, 18.0 mmol) were dissolved in DMF (20 mL) and the mixture was stirred for 2 hours at room temperature. After completion of the reaction, the mixture was concentrated and purified by silica gel chromatography with DCM/(CH₃₀H:THF=1:1)=10%˜40% for 20 minutes to give compound UB-181348d (2.5 g, 74% yield) as yellow solid. LCMS:[M+1]⁺=527.

### Step 4: UB-181348e

Pd(OH)2 (0.1 g) was added to a solution of UB-181348d (2.5 g, 4.8 mmol) in methanol (25 mL). The mixture was stirred at room temperature for 2 hours. The methanol was removed by rotary evaporation and the crude product was washed with dry isopropyl ether (10 mL*3) to give UB-181348e (2.0 g crude) as yellow oil. LCMS:[M+1]⁺=437.

### Step 5: UB-181348f

UB-181348e (2.0 g, 4.6 mmol), (4-aminophenyl)methanol (0.56 g, 4.6 mmol), HATU (2.3 g, 6.0 mmol) and DIEA (0.77 g, 6.0 mmol) were dissolved in DMF (20 mL) and the mixture was stirred for 2 hours at room temperature. After completion of the reaction, the mixture was concentrated and purified by silica gel chromatography with DCM/(CH₃₀H:THF=l:1)=10%˜40% for 20 minutes of elution to give compound UB-181348f (2.1 g, 85% yield) as yellow solid. LCMS:[M+1]=542.

### Step 6: UB-181348g

To a solution of UB-181348f (3.8 g, 7.0 mmol) in methanol (40 mL) was added dropwise hydrochloric acid in dioxane (25 mL, 4N). The mixture was stirred at room temperature for 2 h. The methanol was removed by rotary evaporation and the crude product was washed with dry isopropyl ether (30 mL*3) to give UB-181348g (3.0 g crude) as yellow oil. LCMS: [M+1]⁺=442.

### Step 7: UB-181348h

UB-181348g (50 mg, 0.11 mmol), UB-181348g-1 (123 mg, 0.11 mol), HATU (57 mg, 0.15 mmol) and DIEA (43 mg, 0.33 mmol) were dissolved in DMF (2 mL). The mixture was stirred at room temperature overnight. The crude product was then pre-purified by column chromatography, followed by reversed-phase column purification with 5%0 TFA in H2O/CH₃CN=35%-60%, and the eluate was concentrated and lyophilized to give the desired white solid UB-181348h (62 mg, 36% yield). LCMS: [M+1]⁺=1515.

### Step 8: UB-181348i

UB-181348h (120 mg, 0.08 mmol), bis(4-nitrophenyl)carbonate (46 mg, 0.15 mmol) and DIEA (38.7 mg, 6.0 mmol) were dissolved in DMF (2 mL) and the mixture was stirred at room temperature overnight. DMF was removed by concentration under reduced pressure. The crude product was washed with dry isopropyl ether (5 mL*3) to give UB-181348i (132 mg crude) as yellow oil. LCMS: [M+1]⁺=1681.

### Step 9: UB-181348j

UB-181348i (50 mg, 0.031 mmol), UB-181189 (44.6 mg, 0.052 mol), HOBt (11.0 mg, 0.057 mmol) and DIEA (14.2 mg, 0.11 mmol) were dissolved in DMF (2 mL). The mixture was stirred overnight at room temperature. The crude product was then pre-purified by column chromatography, followed by reversed-phase column purification with 5% o TFA in H2O/CH₃CN=35%-60%, and the eluate was concentrated and lyophilized to give the desired white solid UB-181348j (31.3 mg, 43% yield). LCMS:[1/2M+1]⁺=1201.

### Step 10: UB-181348k

UB-181348j (30 mg, 0.016 mmol) and UB-181320a (18 mg, 0.016 mmol) were dissolved in TEEA/DMF (V/V=1:1, 3 mL). The mixture was stirred at room temperature for 1 hour. The crude product was then purified directly by reversed-phase chromatography (MeOH/H2O=5%˜95%, 45 min). We collected at a concentration of 40% to obtain UB-181348k (8.7 mg, 20% yield) as white solid. LCMS: [1/3M+1]⁺=1174.

### Step 11: UB-181348

UB-181348k (30 mg, 0.016 mmol) was dissolved in TFA/TIPS/H2O/EDT (V/V=92.5/2.5/2.5/2.5, 5 mL). The mixture was stirred at room temperature for 1 hour. The crude product was then purified directly by reversed-phase chromatography (MeOH/H2O=5%˜95%, 45 min). We collected at a concentration of 30% to obtain UB-181348 (9.1 mg, 32% yield) as white solid. LCMS:[1/3M+1]⁺=1034.5.

### Synthesis of Compound UB-181349

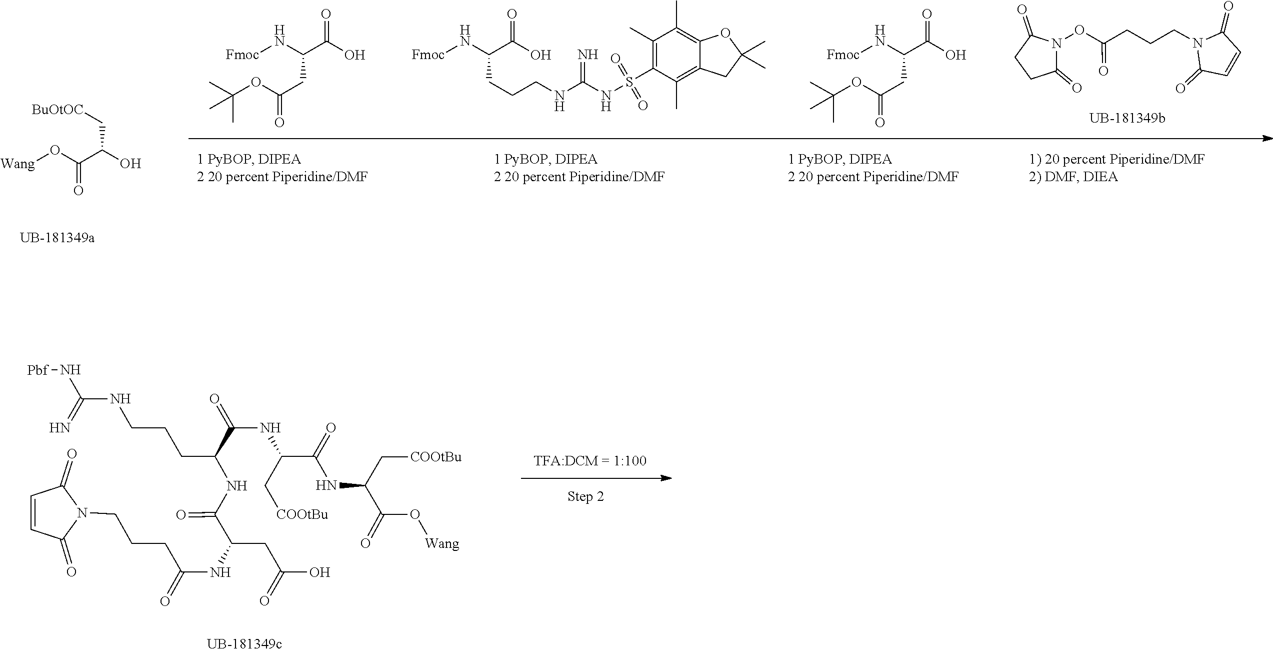

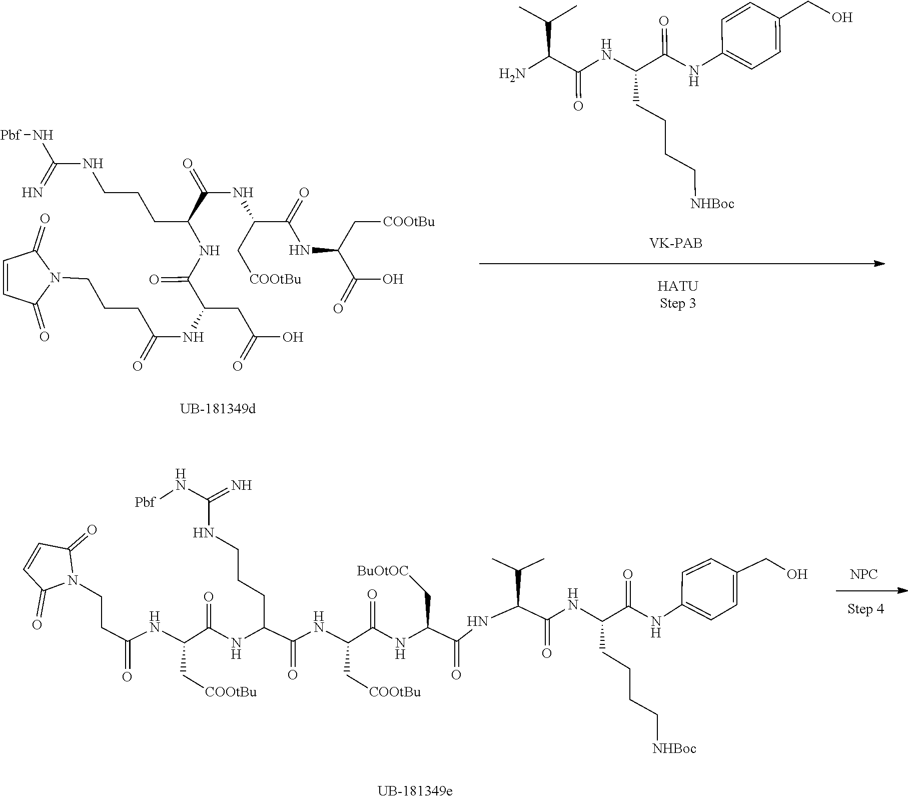

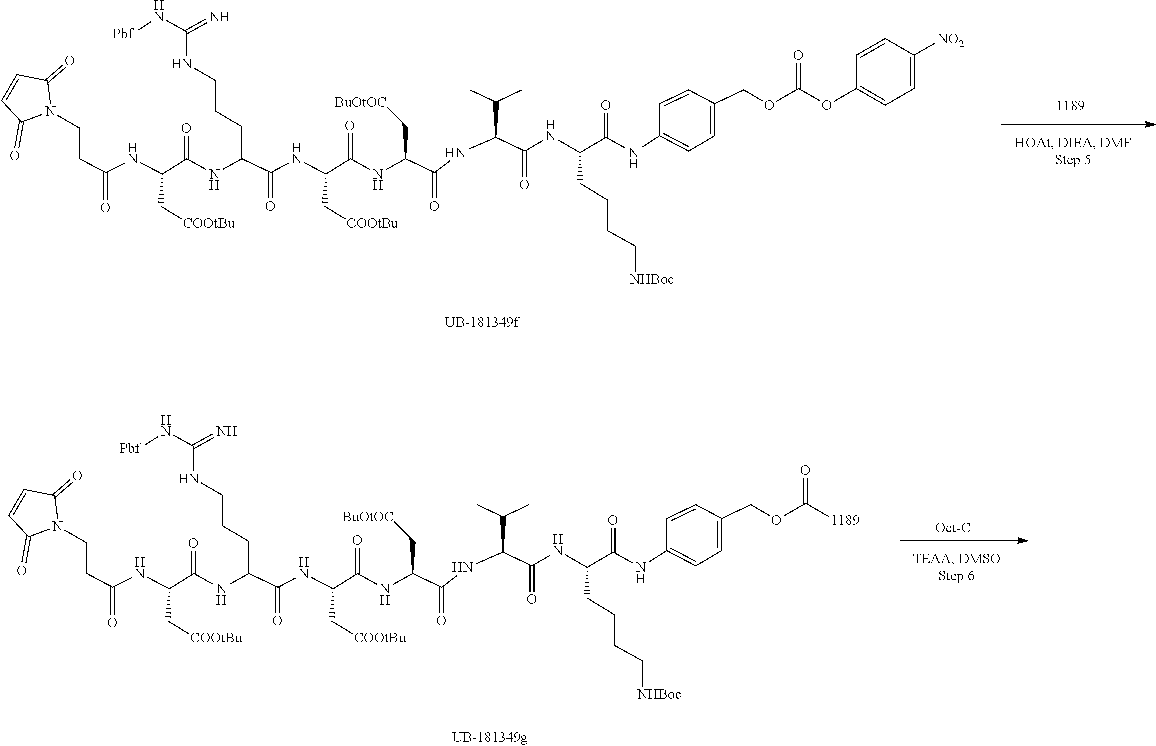

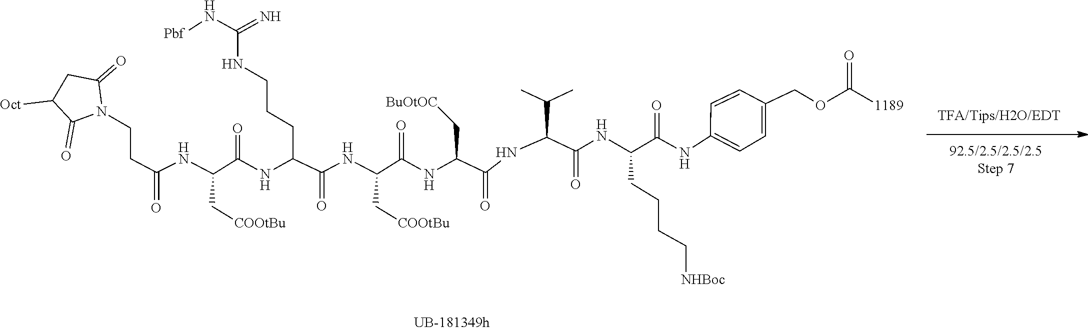

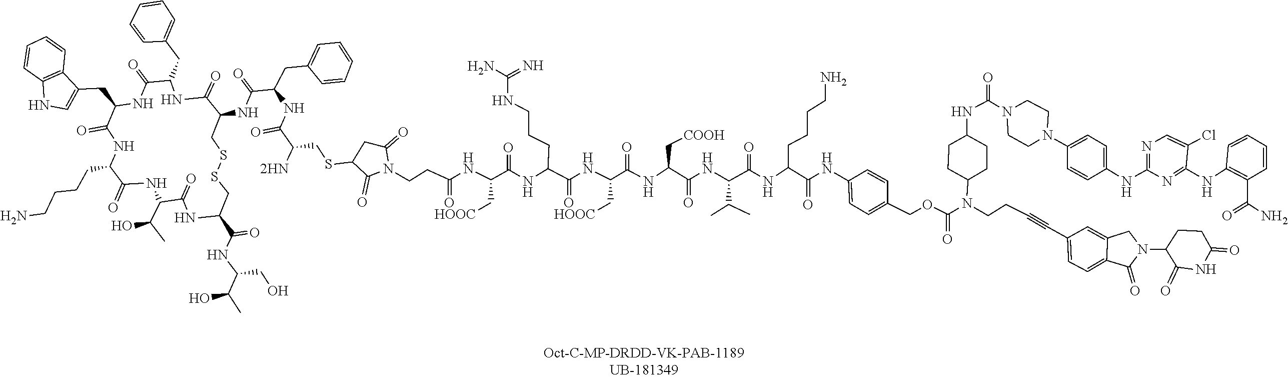

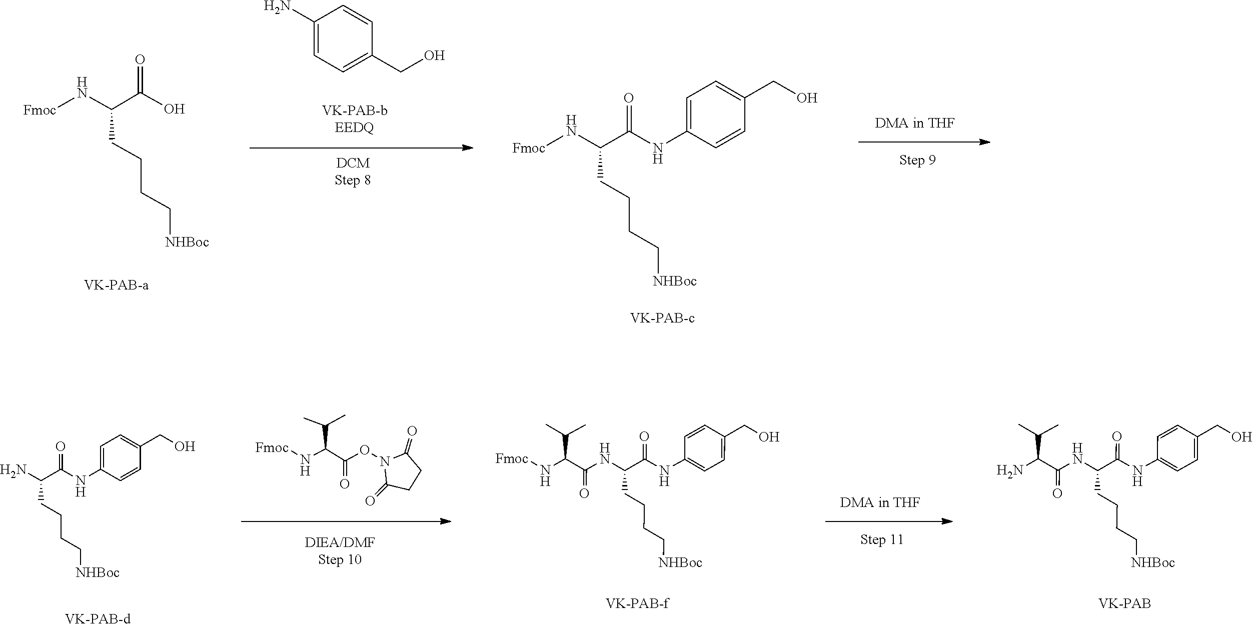

### Step 1: UB-181349c

A resin of compound UB-181349c was placed in a reactor with a filter device, added with 50 mL dichloromethane, and reacted for 1 hour on the shaker. The resin was obtained after filtration and was added to the piperidine/DMF (1:4) solution to react for 15 minutes, and then washed with anhydrous DMF (5×30 mL×0.5 min) to obtain the resin.

N-Fluorenylmethoaycarbonyl-L-aspartate-4-tert-butyl ester (3.8 g, 9.2 mmol), PyBOP (7.2 g, 13.8 mmol), and DIPEA (2.4 mL, 6.9 mL) were dissolved in DMF, and then added with the Resin. The mixture was reacted for 2h on a shaker. Then the mixture was washed with DMF (5×30 mL×0.5 min). The resulting resin was added to piperidine/DMF (1:4) solution and reacted for 15 minutes, and then washed with anhydrous DMF (5×30 mL×0.5 min) to obtain the resin.

Fmoc-D-Arg(Pbf)-OH (6 g, 9.2 mmol), PyBOP (7.2 g, 13.8 mmol), and DIPEA (2.4 mL, 6.9 mL) were dissolved in DMF, and then added with the resin. The mixture was reacted for 2h on a shaker. Then the mixture was washed with DMF (5×30 mL×0.5 min). The resulting resin was added to piperidine/DMF (1:4) solution and reacted for 15 minutes, then washed with anhydrous DMF (5×30 mL×0.5 min) to obtain the resin.

N-Fluorenylmethoaycarbonyl-L-aspartate-4-tert-butyl ester (3.8 g, 9.2 mmol), PyBOP (7.2 g, 13.8 mmol), and DIPEA (2.4 mL, 6.9 mL) were dissolved in DMF, and then added with the Resin. The mixture was reacted for 2h on a shaker. Then the mixture was washed with DMF (5×30 mL×0.5 min). The resulting resin was added to piperidine/DMF (1:4) solution and reacted for 15 minutes, then washed with anhydrous DMF (5×30 mL×0.5 min) to obtain the resin.

UB-181349b (5 g, 18 mmol), and DIEA (522 mg, 18 mmol) were dissolved in DMF, then added with the resin. The mixture was reacted for 2h on a shaker, and then washed with DMF (5×30 mL×0.5 min) to obtain the resin.

### Step 2: UB-181349d

Resin UB-b resin (9 g) was added to TFA: DCM=1:100 solution (90 mL) and stirred for 1.5h. The mixture was filtrated to obtain filtrate. The filtrate subjected to rotary evaporator to remove the solvent to obtain yellow oily crude product, compound UB-181349d (5.1 g). LCMS [M+H]⁺=1049

### Step 3: UB-181349b

Compound UB-181349a (1400 mg, 1.284 mmol) was dissolved in DMF (1 mL), and added with HATU (732 mg, 1.972 mmol), DIEA (331.38 mg, 2.569 mmol) and VK (577 mg, 1.284 mmol). The mixture was stirred at room temperature for 1 hour. The reaction solution was lyophilized by reverse phase column to give the product UB-181349b (1600 mg, 82% yield) as white solid. LCMS [M+H]+=1524.0

### Step 4: UB-181349c

Compound UB-181349b (1600 mg, 1.051 mmol) was dissolved in DMF (10 mL), and added with DIEA (271 mg, 2 mmol) and NPC (479 mg, 1.576 mmol). The mixture was stirred at room temperature for 2 hours and purified by silica gel chromatography (DCM/MeOH=0-50%) to obtain UB-181349c (670 mg, 38% yield) as white solid. LCMS [M+1]+=1690.0

### Step 5: UB-181349d

Compound UB-181349c (526 mg, 0.312 mmol) and DIEA (121 mg, 0.936 mmol) were added to compound 1189 (268 mg, 0.312 mmol), and HOAt (42.4 mg, 0.312 mmol), and dissolved in DMF (5 ml). The mixture was stirred at 30° C. for 4 hours. The mixture was spin-dried and purified by silica gel chromatography DCM/MeOH:THF=1:10˜30% to obtain V3927-070 (UB-181349d, 320 mg, 43% yield) as yellow solid. LCMS [M/2+H]=1206.3

### Step 6: UB-181349e

To a solution of compound UB-181349d (320 mg, 0.133 mmol), and Oct-C (298 mg, 0.266 mmol) in DMSO (6 ml) was added TEAA (3 mL). The mixture was stirred at 30° C. for 0.5 hour. The mixture was spin-dried and purified by reverse phase chromatography MeCN/H2O/50 mmol/1 TEAA to obtain product V3927-073 (UB-181349e, 340 mg, 72% yield) as white solid. LCMS [M/3+H]=1178.6 LCMS[M/2+H]=1767.3

### Step 7: UB-181349

Compound UB-181349e (30 mg, 0.0085 mmol) was dissolved in TFA/TIPS/H2O/EDT (1 ml), and the mixture was stirred at 30° C. for 15 min (repeating 8 times). The reaction solution was quenched with ether (100 ml), stirred at 30° C. for 1.5 h, centrifuged, and left to stand. The supernatant was discarded and the solid was subjected to reversed-phase chromatography (A: 0.5 ml TEA/1 ml TFA/1L H2O B: 0.5% TFA/99.5% MeCN 5%˜35%, 35%˜35%, 35%˜65%) and medium-pressure preparative chromatography (0.1 mol/L NH4OAc/H2O, 2% HOAc/H2O, 80% MeCN/20% H2O/1% HOAc) to give the product V3927-074 (UB-181349, 20 mg, 10% yield) as white solid. LCMS[M/3+H]=1004.81 LCMS[M/2+H]=1506.61

### Step 8: VK-PAB-c

Compound VK-PAB-a (5 g, 10.684 mmol) was dissolved in DCM (10 mL), and added with EEDQ (5.2 g, 21.368 mmol) and VK-PAB-b (1.3 g, 10.684 mmol). The mixture was stirred at room temperature for 1 hour. The reaction solution was washed with DCM (10 mL*3) and the mixture was filtered. The solid was collected to give the product VK-PAB-c (3.8 g, 62% yield) as white solid. LCMS [M+H]+=574.0.

### Step 9: VK-PAB-d

Compound VK-PAB-c (3.8 g, 6.632 mmol) was dissolved in THF (10 mL), and then added with DMA (1.5 g, 33.159 mmol). The mixture was stirred at room temperature for 1 hour. The reaction solution was washed with DCM (10 mL*3) and the mixture was washed with EtO2. The solid was collected to give the product VK-PAB-d (1.2 g, 52% yield) as white solid. LCMS [M+H]+=352.0.

### Step 10: VK-PAB-f

Compound VK-PAB-d (1.2 g, 3.414 mmol) was dissolved in DMF (5 mL), and then DIEA (880.92 mg, 6.829 mmol) and VK-PAB-e (1.48 g, 3.414 mmol) were added. The mixture was stirred at room temperature for 1 hour. The mixture was purified by silica gel chromatography (DCM/MeOH=0˜70%) to obtain VK-PAB-f (1.8 g, 78% yield) as white solid. LCMS [M+1]+=674.0

### Step 11: VK-PAB

Compound VK-PAB-f (1.8 g, 2.229 mmol) was dissolved in THF (20 mL), and then added with DMA (501.63 mg, 11 mmol). The mixture was stirred at room temperature for 1 hour. The mixture was washed with diethyl ether and water, and the aqueous layer was lyophilized to give the product VK-PAB (890 mg, 88.6% yield) as white solid. LCMS [M+H]+=451.0.

### Synthesis of Compound UB-181350

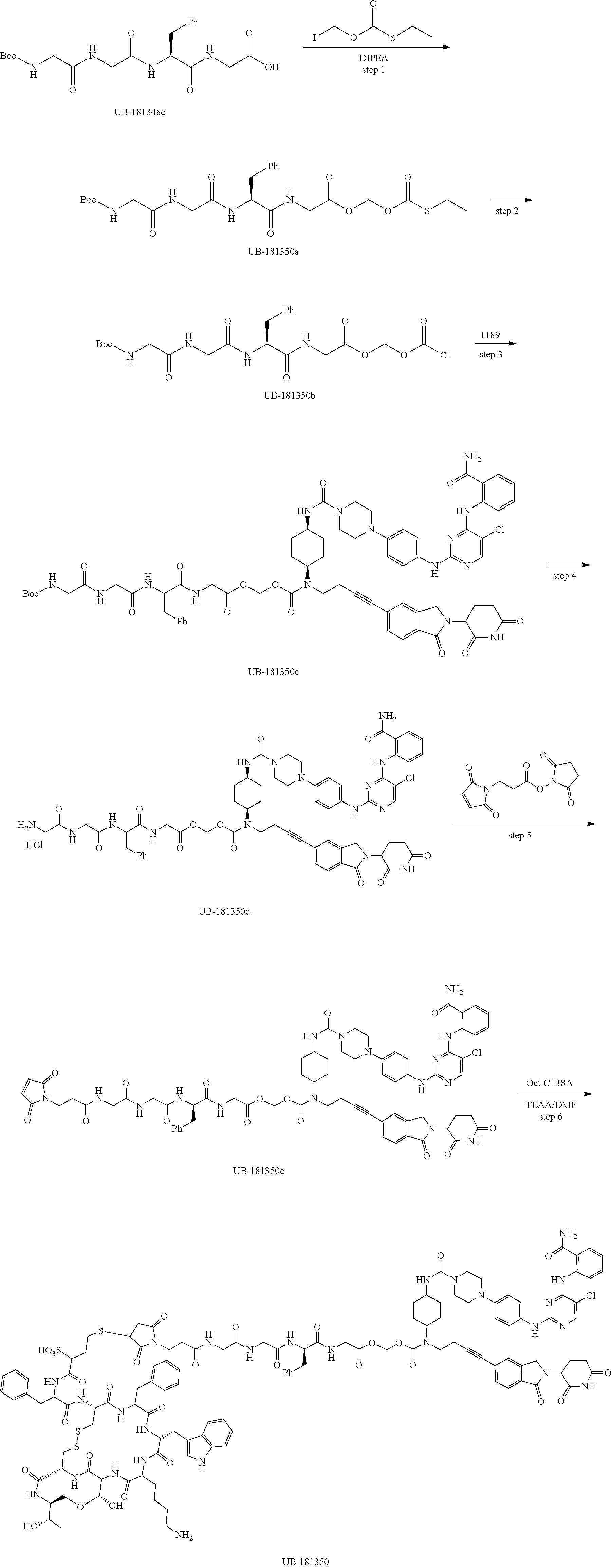

### Step 1: UB-181350a (V3771-088)

UB-181348e (1 g, 2.29 mmol) and S-ethyl O-(iodomethyl)thiocarbonate (0.56 g, 2.29 mmol) were mixed and then dissolved in DCM/H2O (V/V=1:1, 30 ml), and TBAHSO4 (0.78 g, 2.; 29 mmol), NaHCO3 (0.38 g, 4.58 mmol) were added under N2. The mixture was stirred overnight at room temperature. The reaction mixture was poured into water and extracted with DCM (3×20 ml). The organic layers were combined, washed with saline (2×30 ml), dried over anhydrous Na2SO4 and evaporated under reduced pressure to give crude product. The crude product was purified by reversed-phase chromatography to afford UB-181350a (750 mg, 59% yield) as white solid. LCMS: [M+1]⁺=555.

### Step 2: UB-181350b (V3771-110)

UB-181350a (500 mg, 0.9 mmol) was dissolved in 10 mL of dichloromethane, and cooled to −30° C. Sulfuryl chloride (995 mg, 1.8 mmol) was added dropwise and the reaction was stirred for 30 minutes. The reaction mixture was warmed to room temperature. After stirring for another 1 h, the solution was evaporated and left under high vacuum overnight to give 510 mg of crude product UB-181350b. The product was directly used in the next reaction.

### Step 3: UB-181350c

UB-181350b (100 mg, 0.189 mmol), UB-181189 (162 mg, 0.89 mmol), HOBt (110 mg, 0.57 mmol) and DIEA (42 mg, 0.33 mmol) were dissolved in DMF (5 mL). The mixture was stirred overnight at room temperature. The mixture was then concentrated and pre-purified by column chromatography, followed by reversed-phase chromatographic purification using 5‰TFA in H2O/CH₃CN=35% to 60% to elute for 10 min, and then the eluent was concentrated to remove the organic solvent. The remaining aqueous solution was lyophilized to obtain UB-181350c (37 mg, 15% yield) as white solid. LCMS: [M+1]⁺=1352.

### Step 4: UB-181350d

Hydrochloric acid/dichloromethane (25 ml, 4N) was added to a solution of UB-181350c (100 mg, 0.074 mmol) in THF (20 ml). The mixture was stirred at room temperature for 2 h. After removal of THF by evaporation under reduced pressure, the crude was washed with isopropyl ether (30 ml*3) to give UB-181350d (90 mg crude) as white solid. The product was directly used in the next reaction. LCMS: [M+1]⁺=1252.

### Step 5: UB-181350e

UB-181350d (50 mg, 0.066 mmol) and MPOSu (35 mg, 0.132 mmol) were dissolved in DMF (6 mL), and DIEA (18 mg, 0.132 mmol) was added dropwise under N2. The reaction was stirred for 2 hours at room temperature, and TLC showed that the reaction was complete. The crude product was purified by column chromatography to afford UB-181350e (47 mg, yield: 84%) as white solid. LCMS: [M+1]⁺=910.

### Step 6: UB-181350

UB-181350e (50 mg, 0.021 mmol) and UB-181320a (23 mg, 0.021 mmol) were mixed and then dissolved in TEEA/DMF (V/V=1:1, 10 mL). The mixture was stirred at room temperature for 1 h. The crude product was then purified directly by reversed-phase chromatography (MeOH/H2O=5%˜95%, 45 min). We collected at 40% to obtain UB-181350 (28 mg, 37% yield) as white solid. LCMS: [1/3M+1]⁺=868.

### Synthesis of Compound UB-181351

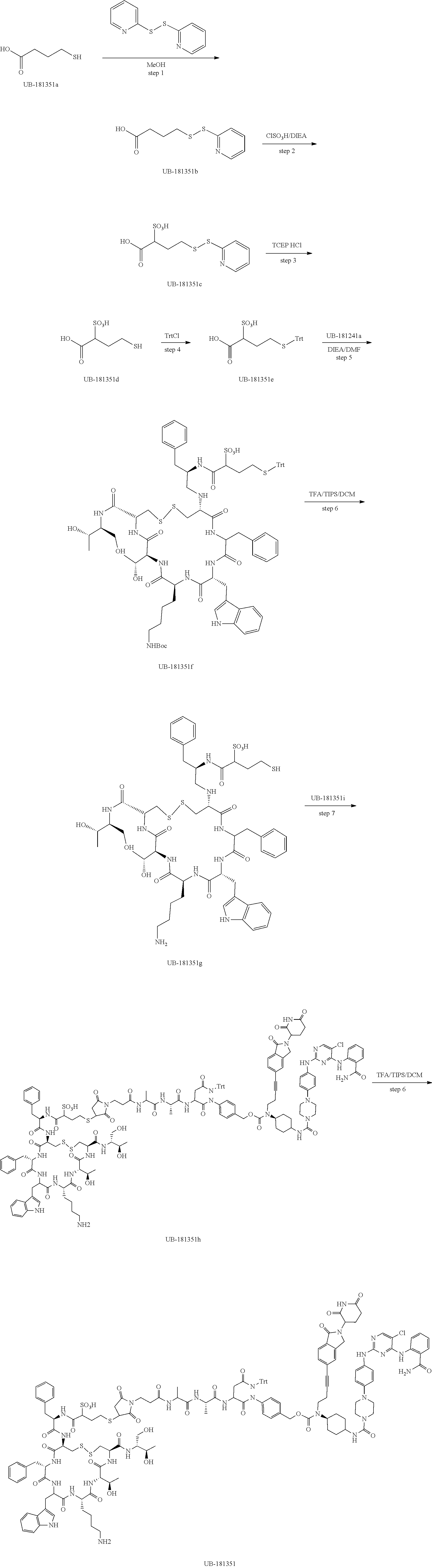

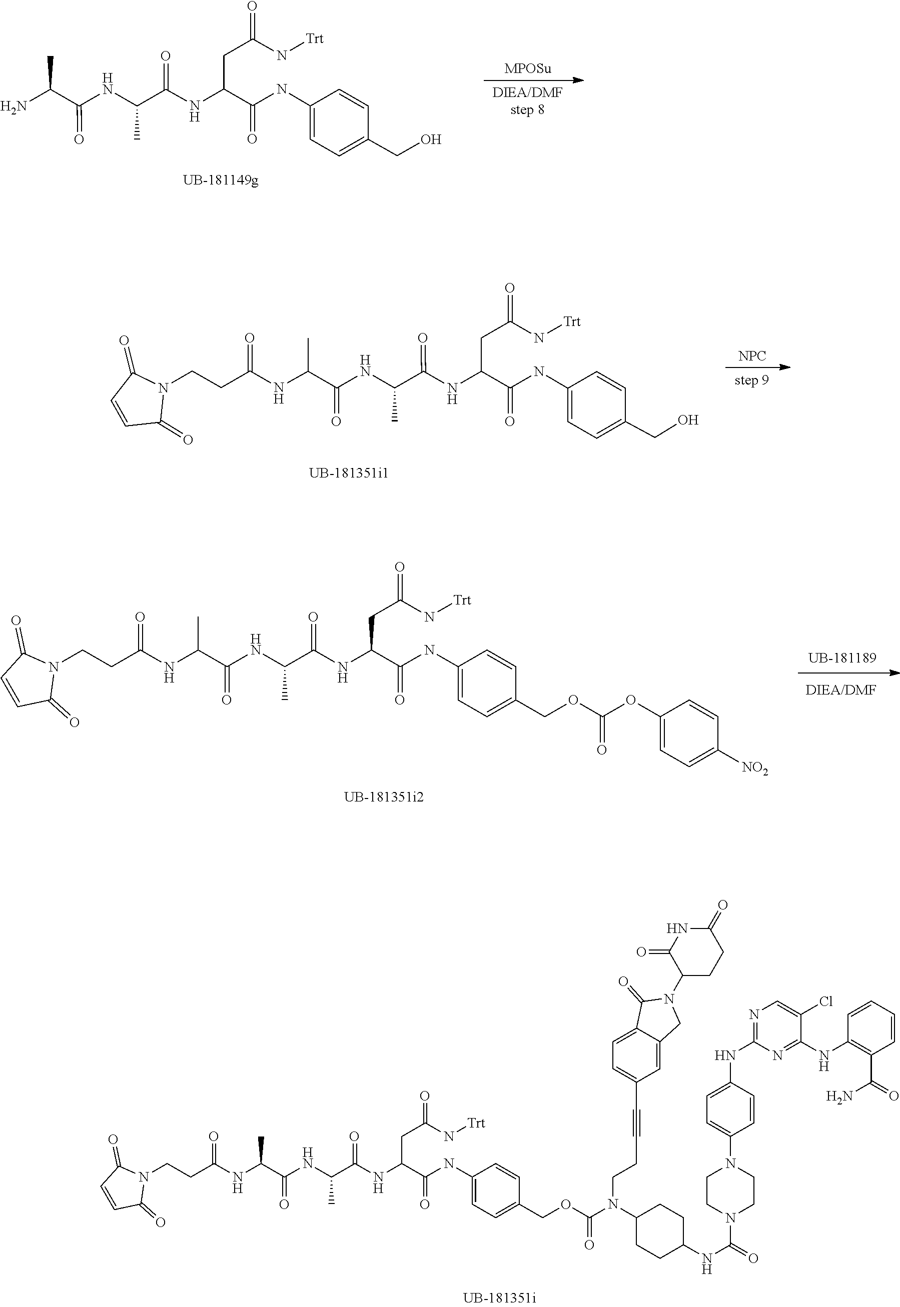

### Step 1: UB-181351b

Compound 2,2′-dithiodipyridine (11000 mg, 50 mmol) was dissolved in methanol (30 mL), and then the mixture was reacted at room temperature for 0.5 hour. The mixture was added with UB-181351a (5000 mg, 41.667 mmol), and then reacted at room temperature for 2 hours. The reaction solution was subjected to reversed phase column chromatography to obtain target product UB-181351b (7.7 g, yield 81% o) as white solid. LCMS[M+H]+=230.0.

### Step 2: UB-181351c

Compound UB-181351b (2700 mg, 11 mmol) was dissolved in dichloroethane (40 mL), and then ClSO₃H (6869 mg, 58 mmol) and DIEA (3041 mg, 23.581 mmol) were added. The mixture was then reacted at 75° C. for 40 minutes. The reaction solution was cooled to room temperature and poured into ice water and the pH was adjusted to 7 with aqueous sodium carbonate solution. The reaction solution was concentrated and then separated by reverse phase column chromatography (MeOH/of AcOH in H2O=25%) to give target product UB-181351c (2.4 g, 66.7% yield) as yellow oil. LCMS [M+H]+=308.0.

### Step 3: UB-181351d

Compound UB-181351c (2 g, 6.4 mmol) was dissolved in tetrahydrofuran (incomplete dissolution) and then PMe3 (1M) was added. The mixture was reacted at room temperature for 30 min until the reaction was clarified. The reaction solution was concentrated to obtain an oil, the oil was washed three times with diethyl ether (50 mL), and the upper layer of diethyl ether was poured off to obtain target crude product UB-181351d (1.3 g, 100% yield) as yellow oil. The crude product was directly used in the next reaction.

### Step 4: UB-181351e

Compound UB-181351d (1300 mg, 6.5 mmol) was dissolved in DMF (10 mL) and then TrtCl (2891 mg, 10.4 mmol) was added. The mixture was reacted at 30° C. for 1.5 hours. The reaction solution was separated by reversed phase column chromatography (acetonitrile/water, collected at 40%) to obtain target product UB-181351e (1.4 g, yield 50%) as yellow solid. LCMS [M+H]+=441.0.

### Step 5: UB-181351f

Compound UB-181241a (700 mg, 0.63 mmol) was dissolved in DMF (3 mL), and then UB-181351e (276 mg, 0.63 mmol), HATU (357 mg, 0.94 mmol) and DIEA (161 mg, 1.25 mmol) were added. After leaving at room temperature overnight, the reaction solution was separated by reversed phase column chromatography to obtain target product UB-181351f (600 mg, yield 62%) as white solid. LCMS [M−H]⁺=1542.1

### Step 6: UB-181351g

Compound UB-181351f (200 mg, 0.13 mmol) was dissolved in DCM/TFA (3.5/1.5 mL), then a catalytic amount of iPr₂SiH was added, and the mixture was reacted at room temperature for 10 min. The reaction solution was pulled dry at low temperature to remove solvent so as to obtain crude product. This crude product was pulped with diethyl ether to obtain target product UB-181351g (45 mg, yield 29%) as yellow solid. LCMS [M−H]⁺=1199.1

### Step 7: UB-181351h

Compound UB-181351g (100 mg, 0.08 mmol) was dissolved in TEAA (1 mL) and a solution of UB-181351i (55 mg, 0.04 mmol) in DMF (2 mL) was added. The reaction solution was reacted for 2 h at room temperature, and then subjected to preparative chromatography to give target product UB-181351h (16 mg, 15% yield) as white solid. LCMS [M/2+H]⁺=1263.3

### Step 8: UB-181351i1

Compound MPOSu (428.3 mg, 1.61 mmol) and UB-181149g (1 g, 1.61 mmol) were dissolved in DMF (10 ml), and then added with DIEA (632.2 mg, 4.83 mmol). The mixture was reacted at room temperature for 2 hours. The reaction solution was concentrated and separated by column chromatography (dichloromethane/methanol=10/1) to obtain target product UB-181351i1 (700 mg, yield 58%) as brown solid. LCMS=[M+H]⁺=774.0.

### Step 9: UB-181351i2

Compound UB-181351i1 (200 mg, 0.26 mmol) and bis(p-nitrophenyl) carbonate (157.3 mg, 0.52 mmol) were dissolved in DMF (2 mL). Then DIEA (66.8 mg, 0.52 mmol) was added. The reaction solution was reacted at room temperature for 2 hours. The reaction solution was concentrated and separated by column chromatography (dichloromethane/methanol=10/1) to obtain target product UB-181351i2 (200 mg, yield 82%) as brown solid. LCMS=[M+H]+=939.1

### Step 10: UB-181351i

Compound UB-181351i2 (100 mg, 0.11 mmol), HOAT (29.0 mg, 0.21 mmol) and UB-181189 (45.7 mg, 0.05 mmol) were dissolved in DMF (1 mL), and then DIEA (41.3 mg, 0.32 mmol) was added. The mixture was reacted at room temperature for 4 hours. The reaction solution was separated by reversed phase column chromatography (water: acetonitrile=0-100%) to obtain target product UB-181351i (51 mg, yield 28.8%) as brown solid. LCMS=[M+H]+=1659.0.

### Step 11: UB-181351

Compound UB-181351h (80 mg, 0.03 mmol) was dissolved in DCM/TFA (3.5/1.5 mL), then a catalytic amount of Pr₂SiH was added, and the mixture was reacted at room temperature for 10 min. The reaction solution was pulled dry at low temperature to remove solvent, and then the residue was subjected to preparative chromatography to obtain target product UB-181351 (28 mg, yield 38%) as white solid. LCMS [M−H]⁺=1306.1

### Synthesis of Compound UB-181352

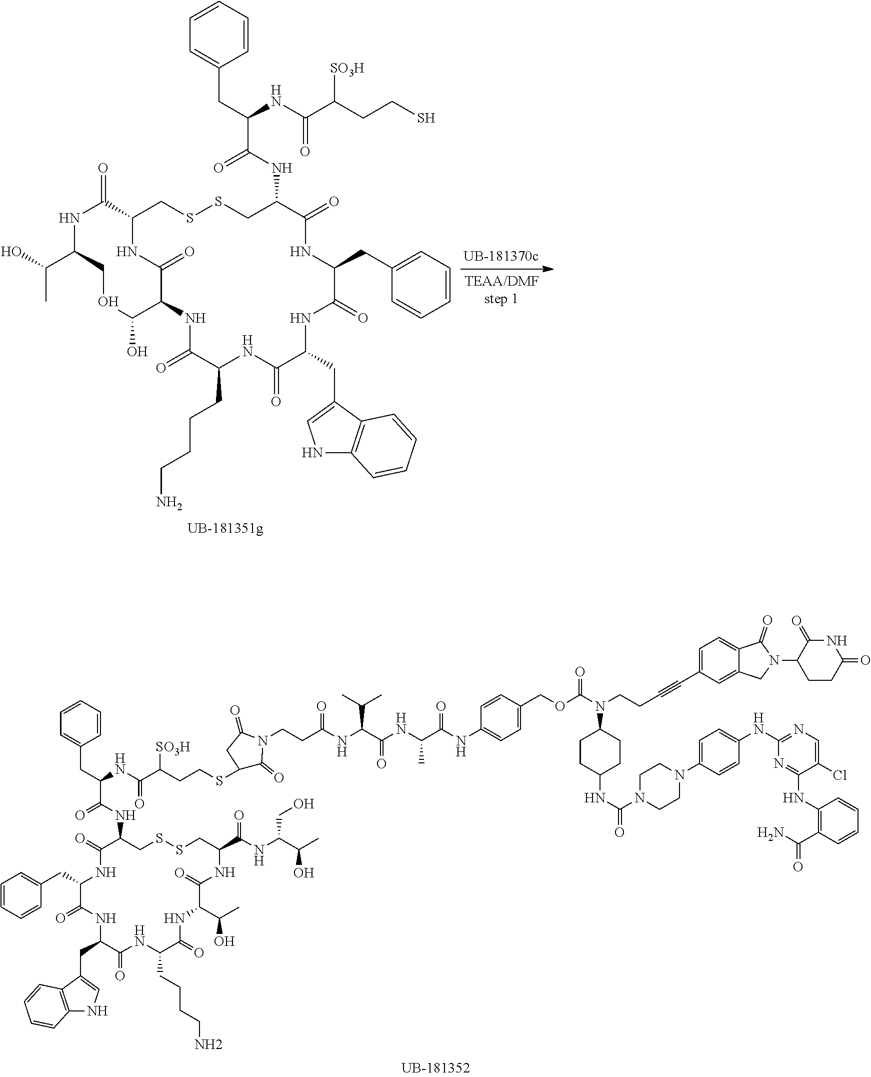

### Step 1: UB-181352

Compound UB-181351g (50 mg, 0.04 mmol) was dissolved in TEAA (0.5 mL), and then added with UB-181370c (28 mg, 0.02 mmol) in DMF (1 mL). The mixture was reacted at room temperature for 2 hours. The reaction solution was separated by reverse phase column chromatography to give 40 mg of crude product. The crude product was subjected to preparative chromatography to obtain target product UB-181352 (18 mg, yield 35%) as white solid. LCMS [M/2−H]⁺=1263.9

### Synthesis of Compound UB-181368

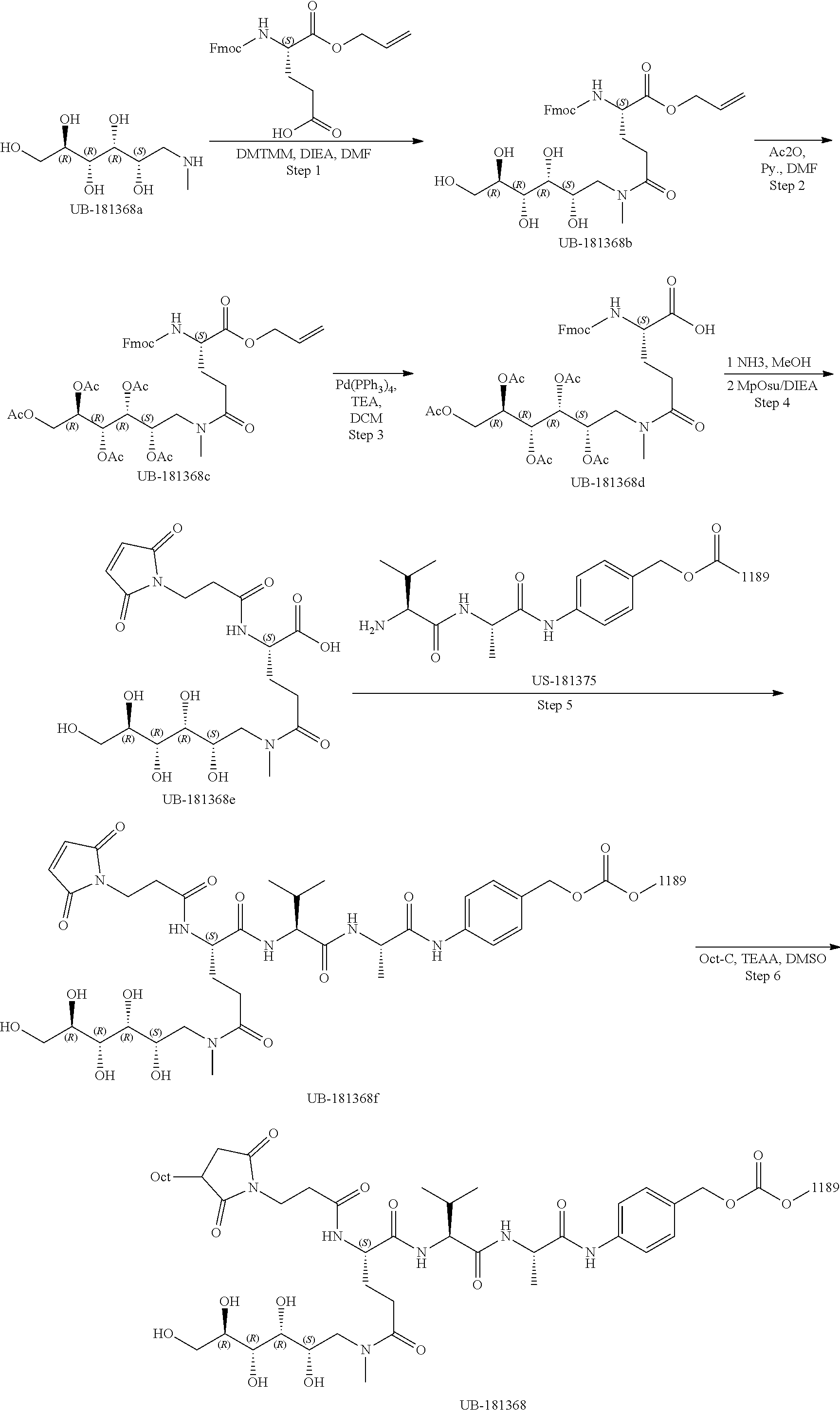

### Step 1: UB-181368b

Compound UB-181368a (10 g, 51.28 mmol) was dissolved in DMF (100 mL), and added with (S)-4-((((9H-fluoren-9-yl)methoxy)carbonyl)amino)-5-(allyloxy)-5-oxopentanoic acid (14 g, 34.19 mmol), DMTMM (13 g, 47.86 mmol) and DIEA (882 mg, 6.84 mmol) under ice bath. The mixture was stirred at room temperature for 1 hour. The reaction solution was spin-dried, and purified by normal phase column (DCM/MeOH=1/10) to give UB-181368b (28 g, 93% yield) as white solid. LCMS[M+H]=587.5.

### Step 2: UB-181368c

Compound UB-181368b (28 g, 47.8 mmol) was dissolved in DMF (60 mL), and added with Ac2O (60 ml), and Py. (2 ml). The mixture was stirred at room temperature for 48 hours. The reaction solution was spin-dried and purified by reversed-phase C-18 column H₂O/CH₃CN=70% to give UB-181368c (24 g, 53% yield) as white solid. LCMS[M+H]=797.6.

### Step 3: UB-181368d

Compound UB-181368c (14.0 g, 17.6 mmol) was dissolved in dry DCM (100 mL), and added with Pd(PPh3)4 (4 g, 3.5 mmol) and TEA (7 g, 70.4 mmol) under nitrogen protection. The mixture was stirred at room temperature for 1 hour. The reaction solution was filtered. The filtrate was spin-dried, and purified by normal phase column DCM/MeOH=0 to 10% to give UB-181368d (12.9 g, 99% yield) as white solid. LCMS[M−H]=755.4.

### Step 4: UB-181368e

Compound UB-181368d (3.2 g, 4.2 mmol) was dissolved in NH3 in MeOH (40 ml). The mixture was reacted at 60 degrees for 1 hour via microwave. The reaction solution was spin-dried, washed with EA (X2) and the precipitate was spin-dried to give yellow solid (V4747-065, 1.36 g, crude). LCMS[M−H]=323.1 The yellow solid V4747-065 (1.36 g, 4.2 mmol) was dissolved in DMF (10 mL), and then MP (3.35 g, 12.6 mmol), and DIEA (2.7 g, 21 mmol) were added. The mixture was stirred at room temperature for 1 hour. The reaction solution was spin-dried and purified by reversed-phase C-18 column 0.1% FA/H2O/MeCN=2% and high-pressure preparative chromatograph to give UB-181368e (72 mg, 3.6% yield) as white solid. LCMS[M−H]=474.2

### Step 5: UB-181368f

Compound UB-181368e (110 mg, 0.232 mmol) was dissolved in DMF (10 mL), and UB-181375 (273 mg, 0.232 mmol), DMTMM (96 mg, 0.348 mmol), DIEA (150 mg, 1.16 mmol), and NMM (70 mg, 0.696 mmol) were added. The mixture was stirred at room temperature for 1 hour. The reaction solution was purified by reversed-phase C-18 column 0.1% TFA/H2O/MeCN to give UB-181368f (220 mg, 58% yield) as yellow solid. LCMS[M+H]=1636.1 LCMS[M/2+H]=818

### Step 6: UB-181368

Compound UB-181368f (220 mg, 0.061 mmol) and Oct-C (137 mg, 0.122 mmol) were dissolved in DMSO (2 mL) and added with TEAA (1 ml). The mixture was stirred at room temperature for 1 hour. The reaction solution was purified by reversed-phase C-18 column (25 mmol TEAA/H2O/MeCN) and medium pressure preparative chromatograph: A: 2% HOAc/H2O B: 80% MeCN/20% H2O/2% HOAc C: 0.1 mmol/L NH4OAc/H2O Si:20%-20% 3CV C, 20%-20% 3CV A, Si (A):S2 (B)=20%-90% 2CV to give UB-181368 (130 mg, 77% yield) as white solid. LCMS[M/2+H]=1380 LCMS[M/3+H]=920

### Synthesis of Compound UB-181369

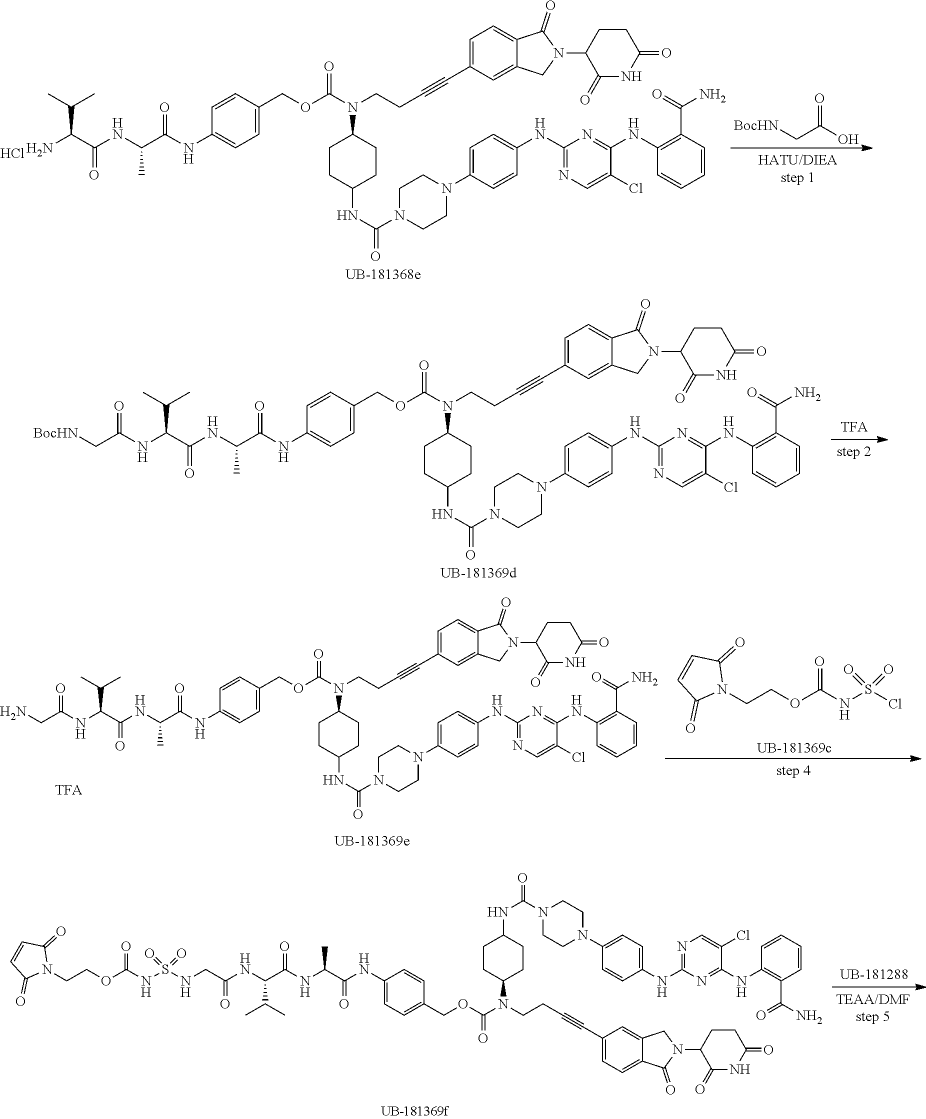

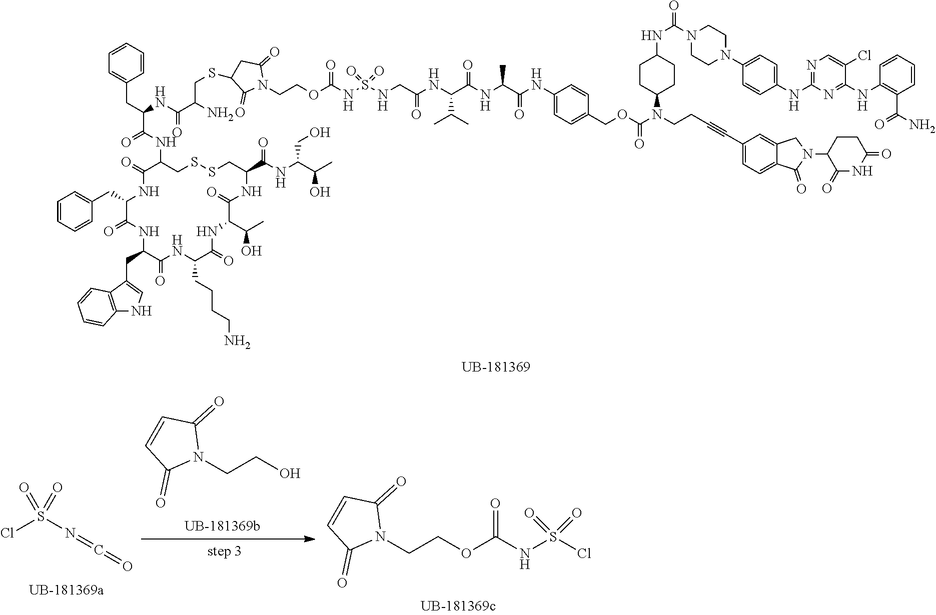

### Step 1: UB-181369d

Compound UB-181368e (2.6 g, 3.6 mmol), Boc-Gly-OH (1.04 g, 3.6 mmol), HATU (2.05 g, 5.4 mmol), and DIPEA (930 mg, 7.2 mmol) were dissolved in dry tetrahydrofuran (50 mL), and then reacted for 1 h at room temperature. The reaction solution was concentrated and separated by column chromatography (dichloromethane/methanol=10/1) to obtain target product UB—181369d (1.8g, yield 73%) as white solid. LC-MS: [M+H]⁺=1335.8.

### Step 2: UB-181369e

Compound UB-181369d (500 mg, 0.37 mmol) was dissolved in TFA/DCM=3/7 (2 mL), and then the mixture was reacted at room temperature for 2 hours. Isopropyl ether (50 ml) was then added, and the mixture was stirred for 10 minutes. The precipitated solid was separated by reverse phase column chromatography (H2O:acetonitrile=0-100%) to obtain target product UB-181369e (328 mg, yield 71%) as white solid. LC-MS: [M+H]⁺=1235.7.

### Step 3: UB-181369c

Compound UB-181369b (100 mg, 0.71 mmol) was dissolved in 15-Crown-5 (3 mL), and then UB-181369a (100 mg, 0.71 mmol) and TEA (72 mg, 0.71 mmol) were added under ice bath. The reaction was then carried out at room temperature for 2 h to afford the target product crude UB-181369c (200 mg, 100% yield), which was used directly in the next reaction.

### Step 4: UB-181369f

Compound UB-181369e (368 mg, 0.3 mmol) was dissolved in 15-crown-5 (18 mL) and then heated to 100° C. until clear. The reaction solution was cooled to room temperature, and added with 4-methylmorpholine (3.68 mL), and then UB-181369c (85 mg, 0.3 mmol) was slowly dripped into the reaction solution and the reaction was carried out for 10 min at room temperature. The reaction solution was separated by reversed phase column chromatography (0.2% trifluoroacetic acid/water/acetonitrile, collected at 36%) to obtain target product UB-181351f (160 mg, yield 36%) as white solid. LC-MS: [M+H]⁺=1481.7.

### Step 5: UB-181369

Compound UB-181288 (65 mg, 0.06 mmol) was dissolved in TEAA (1 mL), and then added with UB-181369f (43 mg, 0.03 mmol) in DMF (2 mL). The mixture was reacted at room temperature for 2 hours. The reaction solution was subjected to medium pressure preparative chromatograph (a system of acetonitrile/0.5% glacial acetic acid/water) to afford target product UB-181369 (30 mg, 40% yield) as white solid. LCMS [M/2+H]⁺=1302.1

### Synthesis of Compound UB-181370

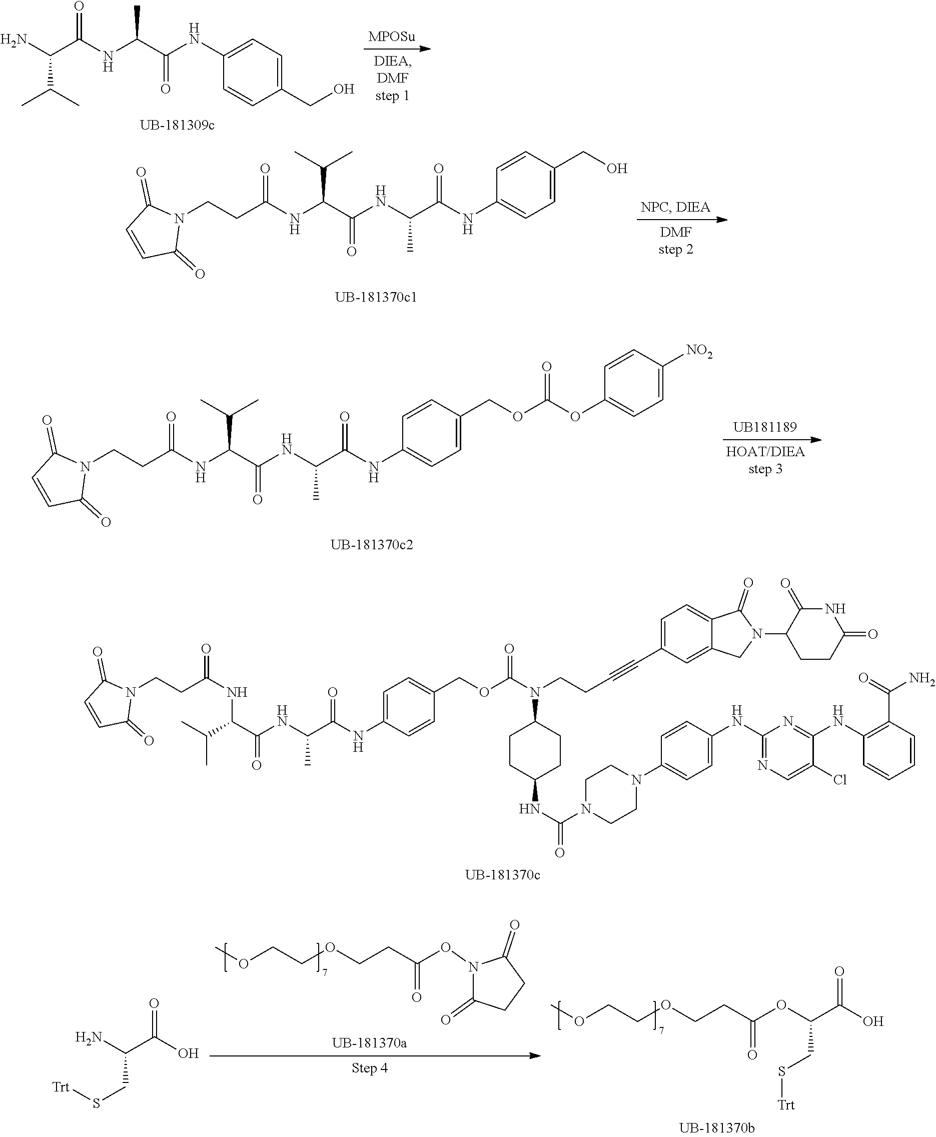

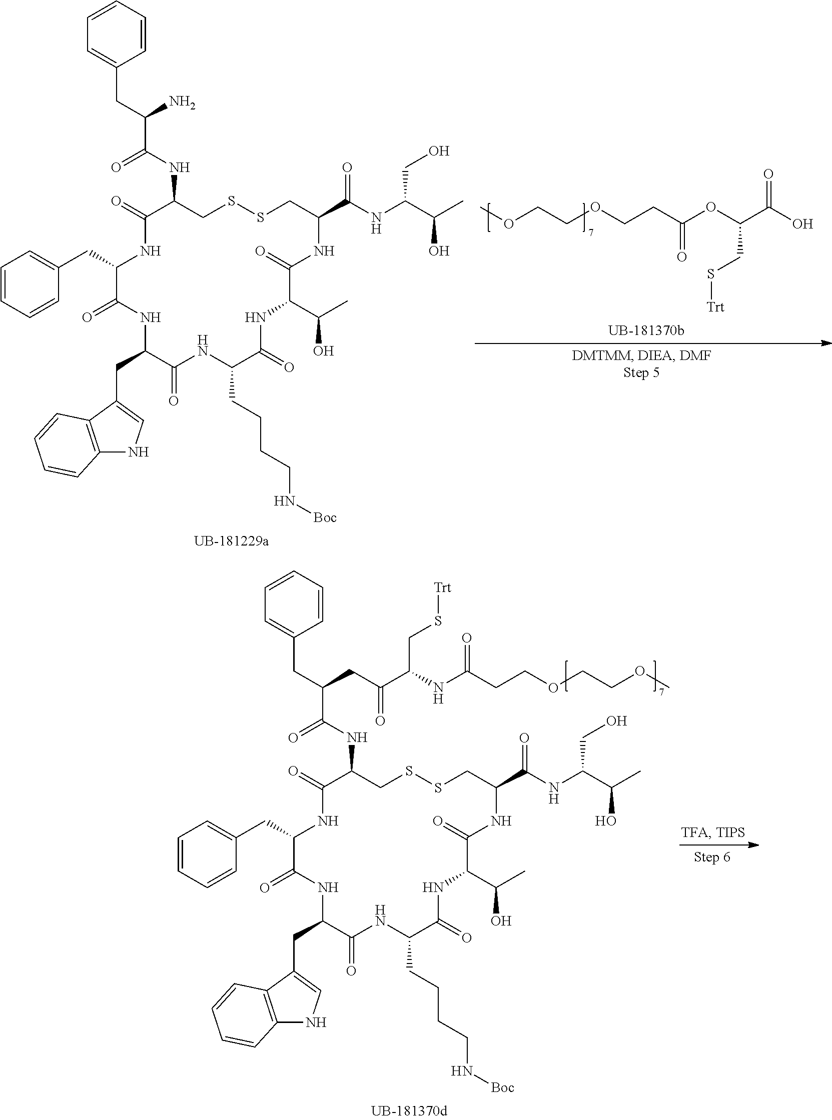

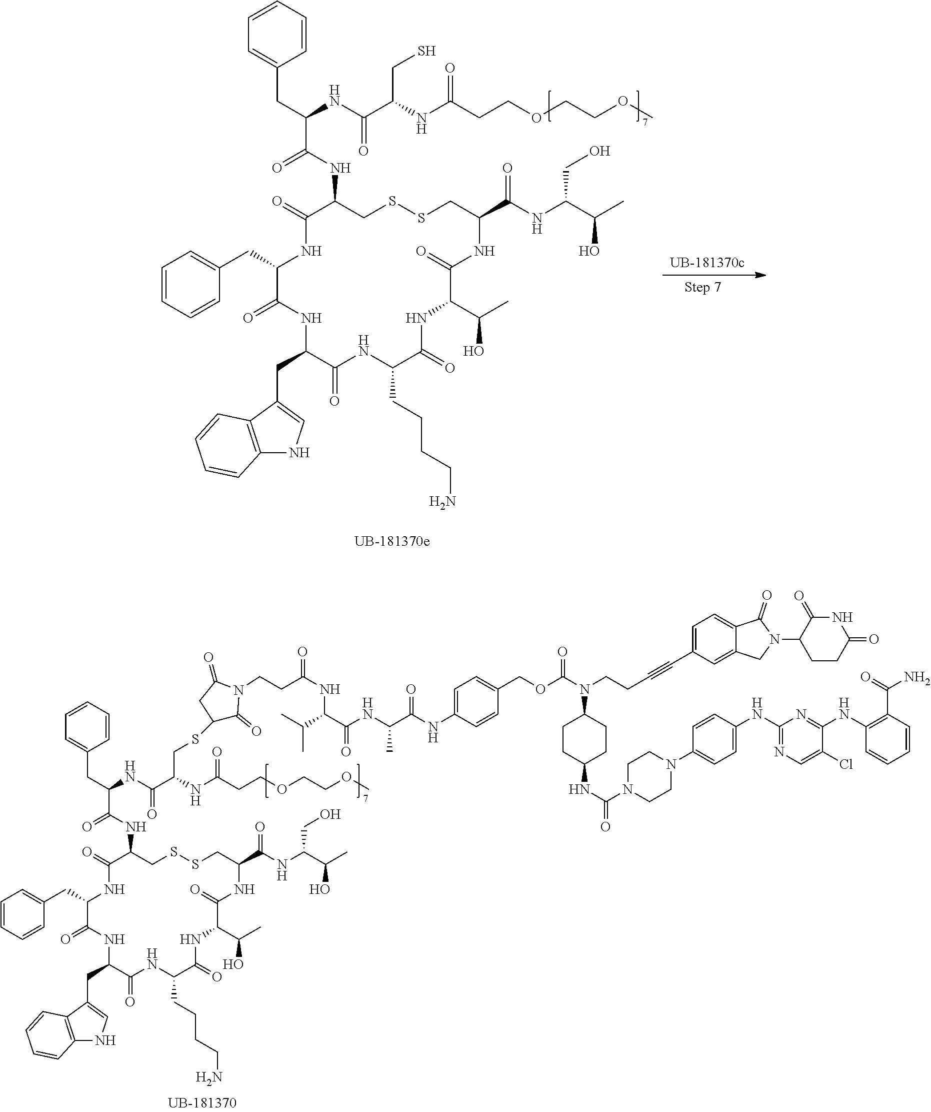

### Step 1: UB-181370c1

UB-181309c (1.8 g, 6.83 mmol) was dissolved in DMF (20 mL), and added with MPOSu (2.0 g, 6.83 mmol) and DIEA (2.6 g, 20.48 mmol). The mixture was stirred at room temperature for 2 hours. The reaction solution was purified by reversed-phase C-18 column H2O/acetonitrile=0-100% to give UB-181370c1 (1.5 g, 50% yield) as yellow solid. LCMS=[M+H]+=445.

### Step 2: UB-181370c2

UB-181370c1 (1.5 g, 3.38 mmol) was dissolved in DMF (20 mL), and added with NPC (2.1 g, 6.76 mmol) and DIEA (0.9 g, 6.76 mmol). The mixture was stirred at room temperature for 2 hours. The reaction solution was spin-dried, and purified by normal phase column DCM/MeOH=0 to 10% to give UB-181370c2 (700 mg, 35% yield) as white solid. LCMS=[M+H]+=610.

### Step 3: UB-181370c

Compound UB-181370c2 (760 mg, 1.2 mmol), HOAT (339.4 mg, 2.5 mmol) and UB 181189 (856.6 mg, 1.0 mmol) were dissolved in DMF (7 mL), and then DIEA (483.0 mg, 3.7 mmol) was added. The mixture was stirred at room temperature for 4 hours. The reaction solution was spin-dried, and purified by normal phase silica gel column (MeOH/DCM=1/10) to give UB-181371c (850 mg, 53% yield) as white solid. LCMS=[M+H]+=1329.0.

### Step 4: UB-181370b

Compound S-trityl-L-cysteine (1.42 g, 3.9 mmol) was dissolved in DMF (10 mL), and added with UB-181370a (2 g, 3.9 mmol), and DIEA (1 g, 7.8 mmol). The mixture was stirred at room temperature for 1 hour. The reaction solution was purified by reversed-phase C-18 column MeCN/H2O=50/50 to give UB-181370b (1.2 g, 41% yield) as yellow oil. LCMS[M−H]=756.4 ¹H NMR (400 MHz, DMSO) δ 12.74 (s, 1H), 8.23 (d, J=8.1 Hz, 1H), 7.37-7.31 (m, 6H) 7.30 (d, J=1.7 Hz, 4H), 7.29-7.23 (m, 5H), 4.18 (td, J=8.2, 5.3 Hz, 1H), 3.58 (t, J=6.6 Hz, 2H), 3.52-3.49 (m, 18H), 3.46 (t, J=5.1 Hz, 8H), 3.44-3.40 (m, 2H), 3.23 (s, 3H), 2.49-2.44 (m, 1H), 2.41-2.33 (m, 3H).

### Step 5: UB-181370d

Compound Oct-Boc (500 mg, 0.45 mmol) was dissolved in DMF (5 mL), and UB-181370b (340 mg, 0.45 mmol), DMTMM (190 mg, 0.675 mmol), and DIEA (120 mg, 0.9 mmol) were added. The mixture was stirred at room temperature for 1 hour. The reaction solution was purified by reversed-phase C-18 column MeCN/H2O=80/20 to give UB-181370d (500 mg, 60% yield) as white solid. LCMS[M+H]=1860.5

### Step 6: UB-181370e

Compound UB-181370d (450 mg, 0.242 mmol) was dissolved in TFA (3.8 mL), TIPS (0.2 ml), and DCM (4 ml). The mixture was stirred at room temperature for 10 min. The reaction solution was spin-dried at 0° C., added with MTBE (50 ml), and centrifuged. The supernatant was poured out and the precipitate was purified by reversed-phase C-18 column with 0.2% FA/H2O/MeCN to give UB-181370e (210 mg, 57% yield) as white solid. LCMS [M+H]=1518.2

### Step 7: UB-181370

Compound UB-181370e (200 mg, 0.132 mmol) and UB-181370c (193 mg, 0.145 mmol) were dissolved in DMSO (2 mL), and added with TEAA (1 ml). The mixture was stirred at room temperature for 1 hour. The reaction solution was purified by Prep-MPLC (0.5% HOAc/H2O/MeCN) to obtain UB-181370 (90 mg, yield 24%) as white solid. LCMS[M/2+H]=1423.3

### Synthesis of Compound UB-181371

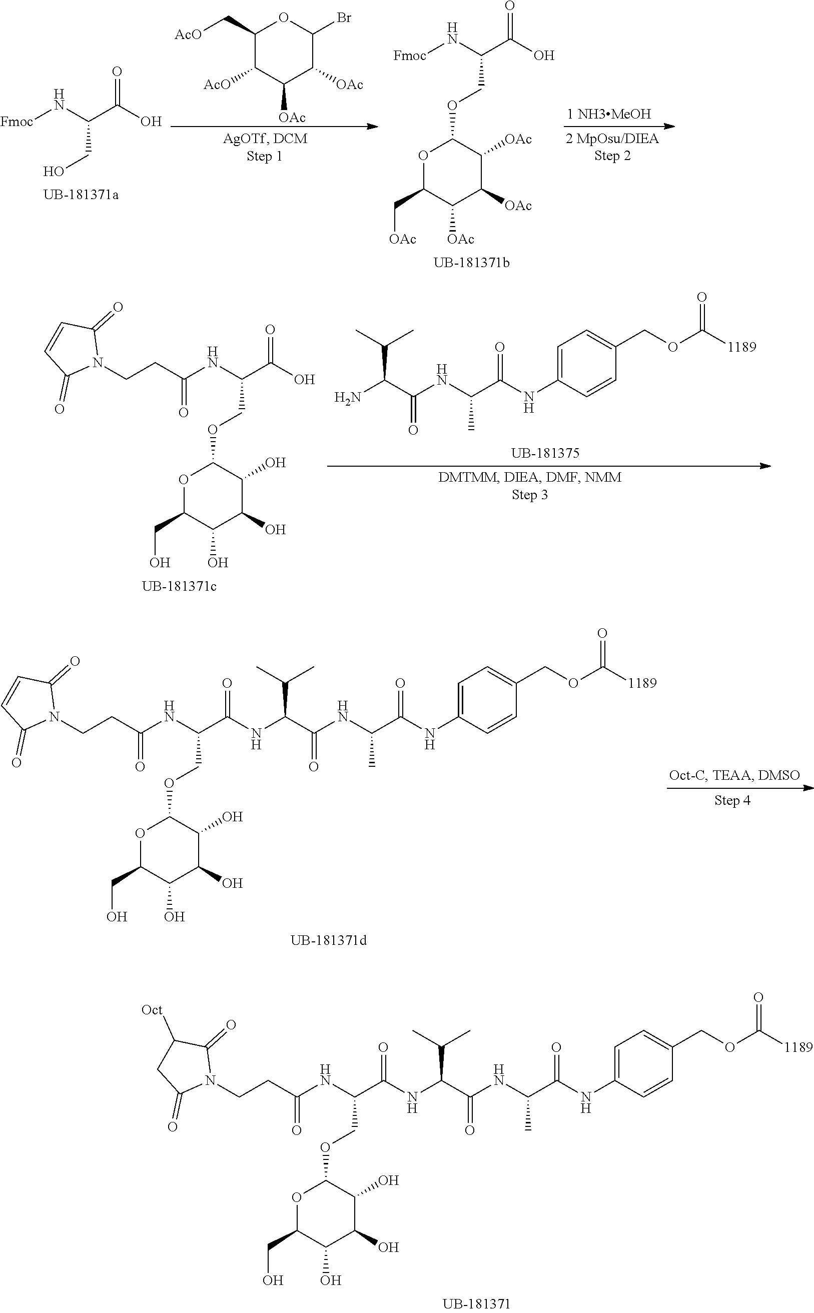

### Step 1: UB-181371b

Compound UB-181371a (20 g, 61.2 mmol), (2R,3R,4S,5R,6R)-2-(acetyloxymethyl)-6-bromotetrahydro-2H-pyran-3,4,5-triethyl triacetate (50 g, 122.3 mmol), and 4A molecular sieve were dissolved in DCM (200 mL), and added with AgOTf (24 g, 91.7 mmol). The mixture was stirred for 1 hour at room temperature under nitrogen protection and protected from light. The reaction solution was filtered, and the filtrate was spin-dried and purified by normal-phase silica gel column with MeOH/DCM=1/10 and reversed-phase C-18 column with MeCN/H2O=0-100% to give UB-181371b (5.6 g, 14% yield) as white solid. LCMS[M+H]=658.5.

### Step 2: UB-181371c

Compound UB-181371b (3 g, 4.6 mmol) was dissolved in NH3 in MeOH (40 ml). The mixture was reacted at 60° C. for 1 hour via microwave. The reaction solution was spin-dried, washed with EA, DCM to obtain white oil (1.23 g, crude). LCMS[M−H]=266 T The white oil (1.23 g, 4.6 mmol) was dissolved in DMF (10 mL), and added with MP (6.118 g, 23 mmol), and DIEA (3 g, 23 mmol). The mixture was stirred at room temperature for 1 hour. The reaction solution was spin-dried and purified by reversed-phase C-18 column 0.1% FA/H2O/MeCN=2% and Prep-HPLC to give UB-181371c (270 mg, 14% yield) as white solid. LCMS[M−H]=417

### Step 3: UB-181371d

Compound UB-181371c (110 mg, 0.26 mmol) was dissolved in DMF (3 mL), and added with UB-181375 (310 mg, 0.26 mmol), DMTMM (108 mg, 0.39 mmol), DIEA (168 mg, 1.3 mmol), and NMM (53 mg, 0.52 mmol). The mixture was stirred at room temperature for 1 hour. The reaction solution was purified by reversed-phase C-18 column 0.1% TFA/H2O/MeCN=40/60 to give UB-181371d (200 mg, 49% yield) as yellow solid. LCMS[M+H]=1578.0

### Step 4: UB-181371

Compound UB-181371d (200 mg, 0.127 mmol) and Oct-C (242 mg, 0.216 mmol) were dissolved in DMSO (2 mL) and added with TEAA (1 ml). The mixture was stirred at room temperature for 1 hour. The reaction solution was purified by reversed-phase C-18 column using 25 mmol TEAA/H2O/MeCN and MPLC: A: 0.5% HOAc/H2O B: 80% MeCN/20% H2O/2% HOAc C: 0.1 mmol/L NH4OAc/H2O Si:20%-20% 3CV C, 20%-20% 3CV A, Si (A):S2 (B)=20%-90% 2CV to give white solid (V4747-109, 40 mg, 30% yield). LCMS[M/2+H]=1350 LCMS[M/3+H]=900

### Synthesis of Compound UB-181375

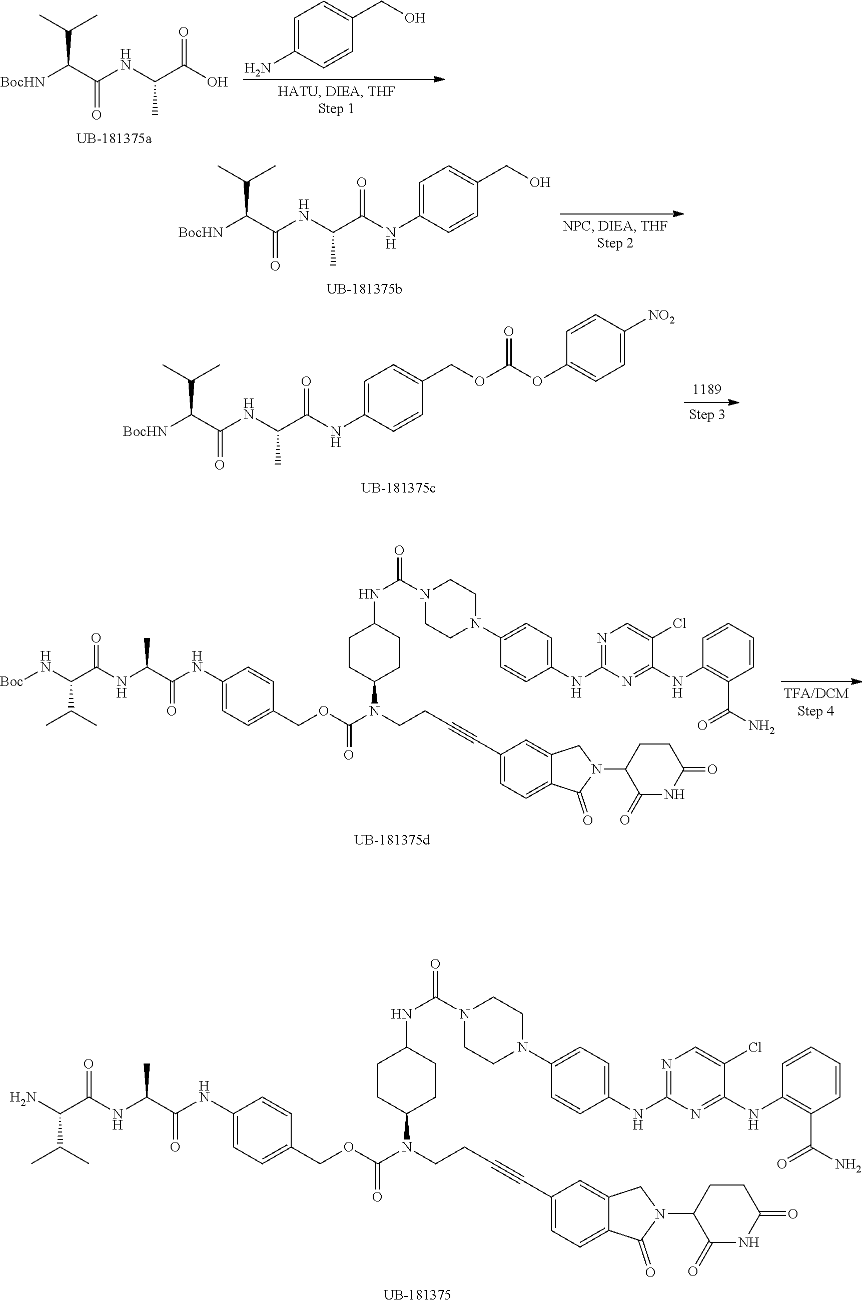

### Step 1: UB-181375b

Compound UB-181375a (20 g, 70 mmol) was dissolved in THF (200 mL), and added with (4-aminophenyl)methanol (10 g, 80 mmol), HATU (34.6 g, 90 mmol) and DIEA (18 g, 140 mmol). The mixture was stirred at room temperature for 1 hour. The reaction solution was filtered. The filtrate was spin-dried, and purified by normal phase column with DCM: (DCM:MeOH:THF=10:1:1)=0-100 to give UB-181375a (30 g, 90% yield) as yellow solid. LCMS[M+H]=394.3

### Step 2: UB-181375c

Compound UB-181375b (3 g, 7.6 mmol) was dissolved in THF (50 mL), and added with NPC (5.8 g, 19 mmol) and DIEA (1.96 g, 15.2 mmol). The mixture was stirred at room temperature for 16 hours. The mixture was spin-dried and purified by silica gel chromatography with PE:EA=1:1 to obtain UB-181375c (3.8 g, 90% yield) as yellow solid. LCMS[M+H]=559.5

### Step 3: UB-181375d

Compound 1189 (1 g, 1.2 mmol), and DIEA (163 mg, 1.2 mmol) were dissolved in DMF (10 mL). The mixture was stirred at room temperature for 2 min. The reaction solution was spin-dried, and purified by normal phase silica gel column with DCM: (DCM:MeOH:THF=10:1:1)=0-100 to give UB-181375d (1.53 g, 70% yield) as yellow solid. LCMS[M+H]=1278.0. ¹H NMR (400 MHz, DMSO) δ 11.86 (s, 1H), 10.99 (s, 1H), 10.13 (s, 1H), 9.54 (s, 3H), 9.24 (s, 1H), 8.80 (s, 1H), 8.31 (s, 1H), 8.16 (s, 1H), 8.12 (d, J=7.0 Hz, 1H), 7.83 (dd, J=7.9, 1.4 Hz, 1H), 7.74 (s, 1H), 7.69 (d, J=7.9 Hz, 1H), 7.61 (d, J=8.5 Hz, 2H), 7.52-7.46 (m, 3H), 7.32 (d, J=8.3 Hz, 2H), 7.13-7.05 (m, 1H), 6.90 (d, J=9.0 Hz, 2H), 6.14 (d, J=5.6 Hz, 1H), 5.07 (d, J=8.8 Hz, 2H), 4.43 (dd, J=15.8, 8.7 Hz, 2H), 4.30 (d, J=17.7 Hz, 1H), 3.83 (dd, J=17.2, 9.8 Hz, 3H), 3.64-3.55 (m, 8H), 3.48 (s, 4H), 3.12 (qd, J=7.3, 4.2 Hz, 8H), 3.03 (s, 3H), 2.71 (d, J=6.3 Hz, 1H), 2.58 (d, J=16.5 Hz, 1H), 2.35 (qd, J=13.2, 4.5 Hz, 1H), 2.02-1.94 (m, 2H), 1.91-1.81 (m, 3H), 1.49 (d, J=6.6 Hz, 3H), 1.40-1.35 (m, 9H), 0.90-0.77 (m, 6H).

### Step 4: UB-18175

Compound UB-181375d (900 mg, 0.70 mmol) was dissolved in DCM (9 mL), and added with TFA (3 mL). The mixture was stirred at room temperature for 15 min under N2 protection. The reaction solution was spin-dried at low temperature, added with ether, and centrifuged. The supernatant was poured off, and the solid was purified by reversed-phase C-18 column to give UB-18175 (350 mg, 42% yield) as white solid. LCMS [M/2+H]⁺=589.3

### Synthesis of Compound UB-181377

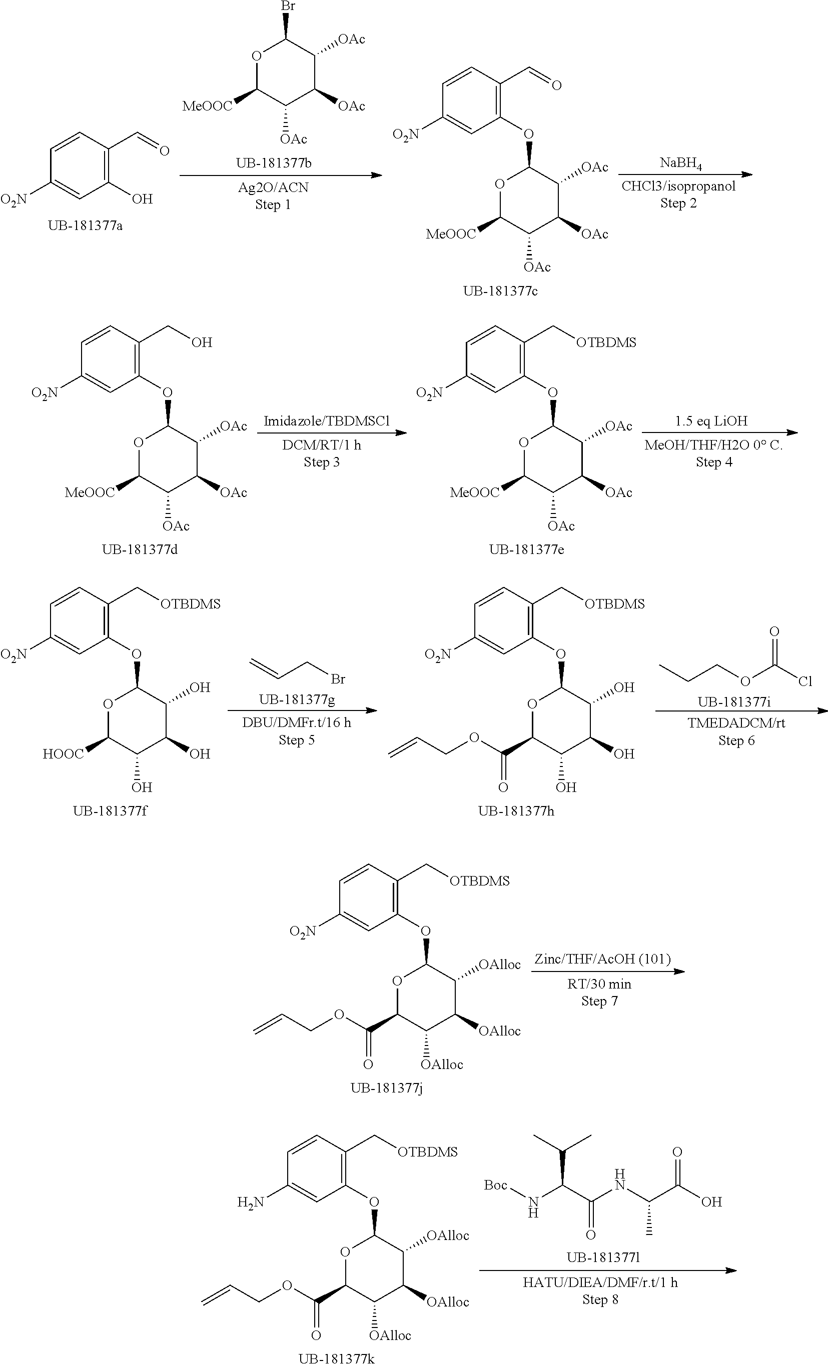

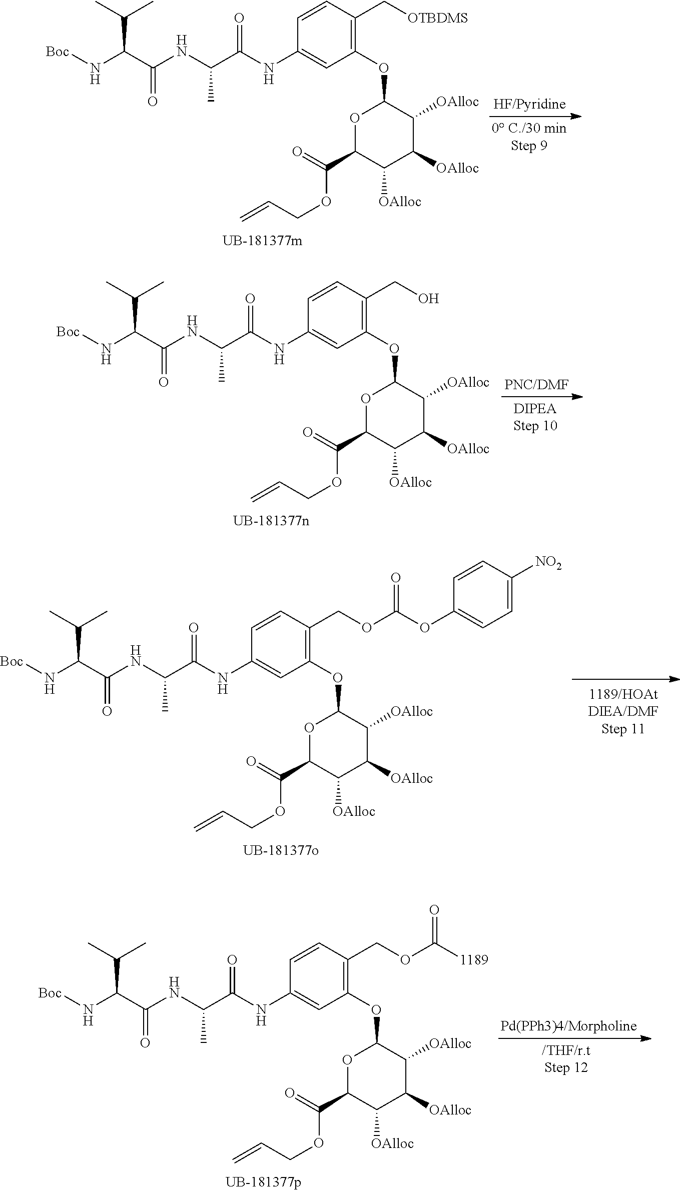

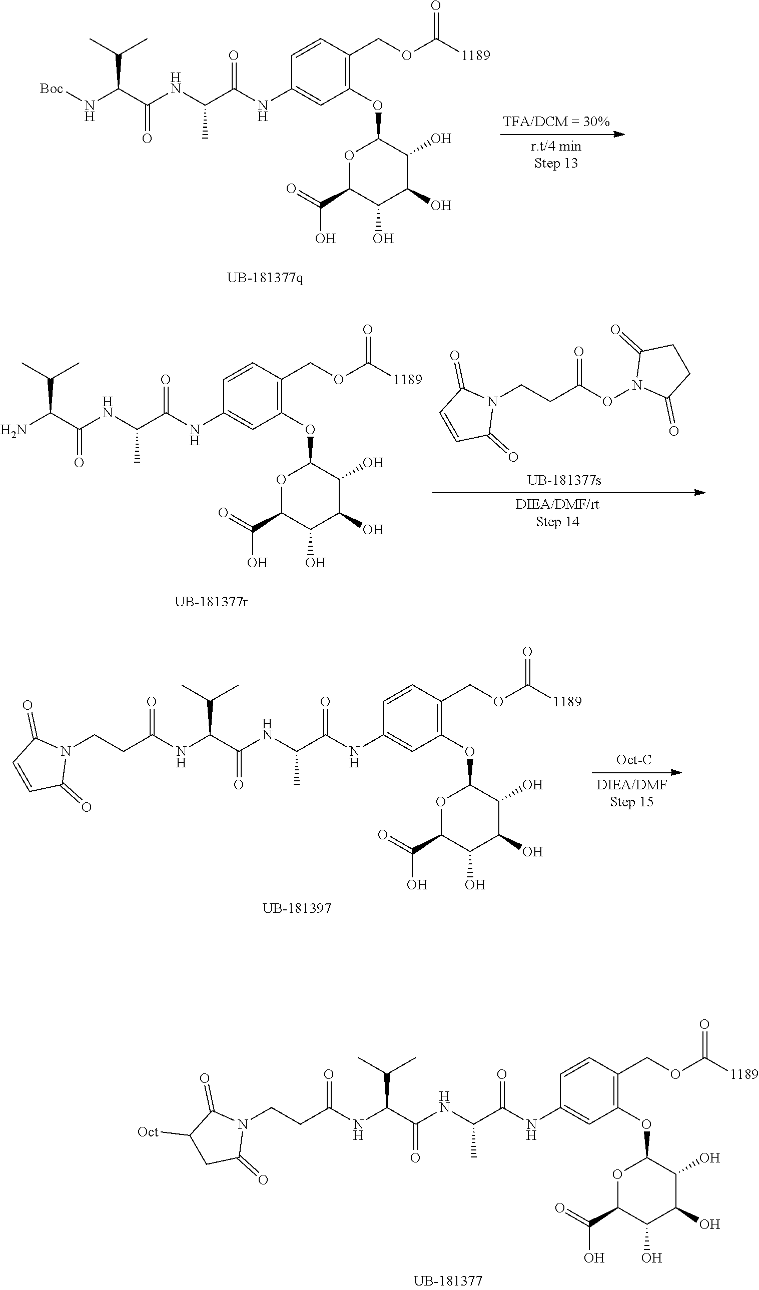

### Step 1: UB-181377c

To a solution of UB-181377a (15 g, 89.8 mmol) in CH₃CN (600 mL) was added UB-181377b (35.47, 89.8 mmol), and Ag₂O (61.8 g, 269.4 mmol) successively. The reaction solution was stirred in dark at room temperature for 18 hours. The reaction solution was filtered to remove solid. The solid was then washed twice with acetonitrile and the resulting filtrate was spin-dried in vacuum. The solid obtained by spin-drying was purified by normal-phase column chromatography (system solvent PE/EtOAc from 70/30 to 50/50) to give UB-181377c (29 g, 93% yield) as yellow solid. Rf=0.28 (PE/EtOAc: 50/50).

### Step 2: UB-181377d

A solution of UB-181377c (23.6 g, 48.9 mmol) and 13.9 g of silica gel in 694 ml of anhydrous CHCl3/isopropanol=5/1 was cooled to 0° C. and added with NaBH4 (2.77 g, 73.3 mmol). The reaction solution was stirred at 0° C. for 45 min under N₂ atmosphere. The mixture was then filtered to remove silica gel and washed with DCM. The mixture was extracted with DCM, washed with saturated saline, dried over anhydrous Na₂SO₄, filtered and concentrated to give crude product. The crude product was purified by silica gel chromatography (PE/EA=1/1) to obtain UB-181377d (16.3g, 69% yield) as white solid. LCMS [M+H]⁺=no mass. ¹H NMR (400 MHz, DMSO) δ 8.02 (dd, J=8.4, 2.1 Hz, 1H), 7.86 (d, J=2.2 Hz, 1H), 7.71 (d, J=8.5 Hz, 1H), 5.84 (d, J=7.7 Hz, 1H), 5.47 (t, J=9.6 Hz, 1H), 5.21-5.09 (m, 2H), 4.82 (d, J=9.8 Hz, 1H), 4.47 (q, J=16.4 Hz, 2H), 3.65 (s, 3H), 2.06-2.00 (n, 9H).

### Step 3: UB-181377e

To a solution of UB-181377d (12 g, 24.8 mmol) and imidazole (3.4 g, 49.7 mmol) in DCM (500 mL) was added TBDMSCl (7.5 g, 49.7 mmol) and the solution was stirred for 1 h at room temperature. The reaction was quenched by adding saturated aqueous NaHCO₃ and extracted with DCM. The combined organic layer was dried over MgSO4, filtered, and concentrated under reduced pressure. The residue was purified by silica gel column chromatography (gradient eluent PE/EtOAc from 80/20 to 60/40) to obtain compound UB-181377f as white solid (14 g, 94% yield). LCMS [M+1]⁺=600.5. ¹H NMR (400 MHz, DMSO) δ 8.04 (dd, J=8.5, 1.6 Hz, 1H), 7.87 (d, J=2.1 Hz, 1H), 7.67 (d, J=8.5 Hz, 1H), 5.86 (d, J=7.7 Hz, 1H), 5.48 (t, J=9.6 Hz, 1H), 5.25-5.07 (m, 2H), 4.83 (d, J=9.8 Hz, 1H), 4.67 (dd, J=36.9, 16.3 Hz, 2H), 3.64 (s, 3H), 2.01 (dd, J=10.7, 8.6 Hz, 9H), 0.92 (s, 9H), 0.15-0.06 (m, 6H).

### Step 4: UB-181377f

To a solution of UB-181377e (18 g, 30.1 mmol) in MeOH/THF was added LiOH/H₂O (1.9 g, 45.1 mmol) at 0° C. The mixture was stirred at 0° C. for 1 hour. To the mixture was added AcOH (ph=6). The mixture was concentrated and purified by silica gel column chromatography (DCM/MeOH=0 to 25%) to obtain UB-181377f (12.1 g, 88% yield). LCMS [M+H]⁺=460.3. ¹H NMR (400 MHz, DMSO-d₆) δ 7.85 (dd, J=8.4, 2.2 Hz, 1H), 7.75 (d, J=2.2 Hz, 1H), 7.59-7.40 (m, 1H), 4.95 (d, J=7.3 Hz, 1H), 4.85-4.76 (m, 1H), 4.72-4.58 (m, 1H), 3.55 (d, J=9.6 Hz, 1H), 3.25-3.03 (m, 3H), 0.82 (s, 8H), 0.00 (d, J=3.3 Hz,

H).

### Step 5: UB-181377h

A solution of UB-181377f (200 mg, 0.4 mmol), and DBU (132.5 mg, 0.9 mmol) in DMF (2 mL) was stirred at room temperature for 30 min and added with UB-81377g (105.4 mg, 0.9 mmol). The mixture was stirred at 50° C. for 16 hours. The mixture was concentrated under reduced pressure, and purified by silica gel column chromatography (MeOH/DCM=1/10) to obtain UB-181377h (100 mg, 46% yield). LCMS [M+H]⁺=500.3. ¹H NMR (400 MHz, DMSO-d₆) δ 7.88 (dd, J=8.4, 2.2 Hz, 1H), 7.78 (d, J=2.2 Hz, 1H), 7.55-7.46 (m, 1H), 5.79 (ddt, J=17.2, 10.5, 5.2 Hz, 1H), 5.51 (d, J=4.8 Hz, 1H), 5.36 (d, J=5.7 Hz, 1H), 5.25-4.97 (m, 4H), 4.79 (dd, J=16.3, 1.1 Hz, 1H), 4.66 (dd, J=16.3, 1.1 Hz, 1H), 4.59-4.36 (m, 2H), 4.08 (d, J=9.6 Hz, 1H), 3.35 (td, J=9.0, 5.5 Hz, 1H), 3.29-3.23 (m, 2H), 0.82 (s, 9H), −0.00 (d, J=0.9 Hz, 6H).

### Step 6: UB-181377j

To a solution of UB-181377h (740 mg, 1.5 mmol) and TMEDA (1.1 ml, 7.5 mmol) in anhydrous DCM (10 ml) was added UB-81377i (2.7 ml, 44.5 mmol) and stirred for 1 hour at room temperature. The mixture was then filtered over a Celite® pad to remove solids. The organic solvent was concentrated and the crude product was purified by silica gel column chromatography (eluent PE/EA:80/20) to afford UB-181377h (1 g, 92% yield) as yellow oil. LC-MS: [M+H]⁺=752.6. ¹H NMR (400 MHz, DMSO-d₆) δ 7.95 (dd, J=8.4, 2.1 Hz, 1H), 7.80 (d, J=2.2 Hz, 1H), 7.58 (d, J=8.8 Hz, 1H), 6.00-5.57 (m, 5H), 5.32 (t, J=9.4 Hz, 1H), 5.26-5.01 (m, 10H), 4.82 (d, J=9.9 Hz, 1H), 4.72-4.32 (m, 10H), 0.80 (d, J=11.2 Hz, 9H), −0.02 (dd, J=16.5, 2.6 Hz, 6H).

### Step 7: UB-181377k

To a stirred solution of UB-181377j (3.78 g, 5 mmol) in THF/AcOH (20 mL/4 ml, 5:1 v/v) was added active zinc powder (16.4 g, 250 mmol) for one time. The resulting suspension was stirred vigorously under argon for 1 hour at room temperature. The mixture was purified by reversed phase column (H2O:acetonitrile=0-81%). The resulting H₂O/ACN solution was extracted twice with EA (40 ml). EA was concentrated to give UB-181377k (2.7 g, 75% yield). LC-MS: [M+H]⁺=722.5.

### Step 8: UB-181377m

A mixture of UB-181377k (2.6 g, 3.6 mmol), UB-81377L (1.04 g, 3-6 mmol) and HATU (2.05 g, 5.4 mmol) and DIPEA (930 mg, 7.2 mmol) in dry THF (50 ml) was stirred for 0.5 hour at room temperature. The mixture was concentrated and purified by silica gel column chromatography (DCM/MeOH:10/1) to obtain UB-181377m (4.8 g, 100% yield) as yellow oil. LC-MS: [M+H]⁺=993.8.

### Step 9: UB-181377n

HF/pyridine 70% (17 ml) was added dropwise to a solution of UB-181377m (4.5 g, 4.5 mmol) in anhydrous THF (50 ml), and the mixture was stirred for 2 hours at room temperature under argon. Then the reaction was quenched with saturated NaHCO₃ solution (30 ml) and extracted with EtOAc (3×40 ml). The combined organic layer was dried over MgSO4, filtered, and concentrated under reduced pressure. The crude was purified by silica gel column chromatography (eluent DCM/MeOH: 15/1) to obtain UB-181377n (2.44 g, 63% yield) as colorless oil. LC-MS: [M+H]⁺=878.6.

### Step 10: UB-181377o

To a solution of UB-181377n (2.4 g, 2.7 mmol) and PNC (2.5 g, 8.2 mmol) in dry DMF (10 mL) was added DIEA (1.06 g, 8.2 mmol), and the solution was stirred for 1 hour at room temperature. The mixture was added with H₂O, extracted with EtOAc (3×40 mL), and the crude was purified by silica gel column chromatography (eluent DCM/MeOH:15/1) to afford UB-81377o (2.55 g, 90% yield) as white solid. LC-MS: [M+H]⁺=1043.9.

### Step 11: UB-181377p

To a solution of 1189 (1.4 g, 1.6 mmol) and DIEA (6.7 mg, 4.6 mmol) in DMF (5 ml) was added UB-181377o (2.45 g, 2.3 mmol) and HOAT (320 mg, 2.3 mmol). The mixture was stirred at 30° C. for 4 hour. The mixture was added with H₂O, extracted with EtOAc (3×40 mL), and the crude was purified by silica gel column chromatography (DCM/MeOH:15/1) to afford UB-181377p (1.05 g, 25% yield) as white solid. LC-MS: [1/2M+H]⁺=881.

### Step 12: UB-181377q

UB-181377p (400 mg, 0.2 mmol) was dissolved in anhydrous THF (20 ml). A solution of MOR (200 mg, 2.0 mmol, 10 eq.) was added to THF (50 ul) and the mixture was stirred under argon for 10 min, followed by the addition of Pd (PPh3)₄ (52 mg, 0.04 mmol). The solution was stirred for 30 minutes at 0° C. The mixture was purified by reversed-phase column with (H2O:acetonitrile=0-100%) to give UB-181377q (150 mg, 45% yield) as white solid. LC-MS: [M+H]⁺=1469.2.

### Step 13: UB-181377r

A mixture of UB-181377q (50 mg, 0.035 mol) in TFA/DCM=30% (2 mL) was stirred for 4 min. Isopropyl ether (50 mL) was added to the mixture, and the solution was stirred for 10 minutes. The mixture was centrifuged and the solid was purified by reversed-phase column with (H2O:acetonitrile=0-100%) to give UB-81377R (30 mg, 70% yield) as white solid. LC-MS: [M+H]⁺=1369.2.

### Step 14: UB-181397

To a mixture of UB-181377r (220 mg, 0.16 mmol) and UB-81377S (86 mg, 0.32 mmol) in DMF (5 ml) was added DIEA (41 mg, 0.32 mmol). The mixture was stirred at room temperature for 30 min. The mixture was purified by reversed-phase column with (H2O (0.5% AcOH): acetonitrile=40%) to give UB-181397 (105 mg, 44% yield) as white solid. LC-MS: [M+H]⁺=1522.2.

### Step 15: UB-181377

To a solution of UB-181397 (10 mg, 0.006 mmol) and Oct-C (15 mg, 0.012 mmol) in DMF (1 ml) was added with DIEA (2 mg, 0.012 mmol). The mixture was stirred at 30° C. for 1 hour. The mixture was purified by reversed-phase column with (H2O (0.5% AcOH): acetonitrile=0-100%) to give UB-181377 (2 mg, 16% yield) as white solid. LC-MS: [1/3M+H]⁺=881.4.

### Synthesis of Compound UB-181378

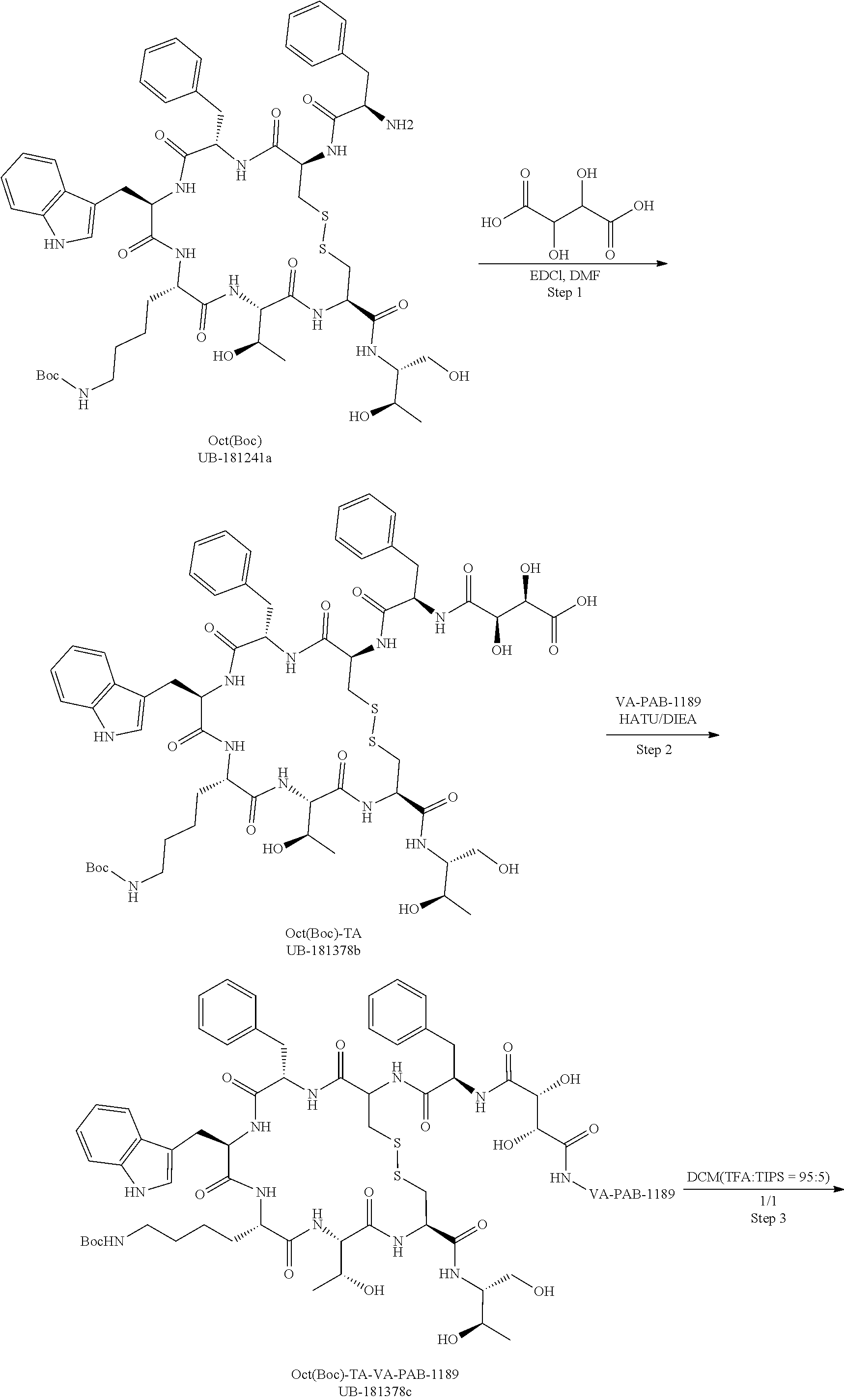

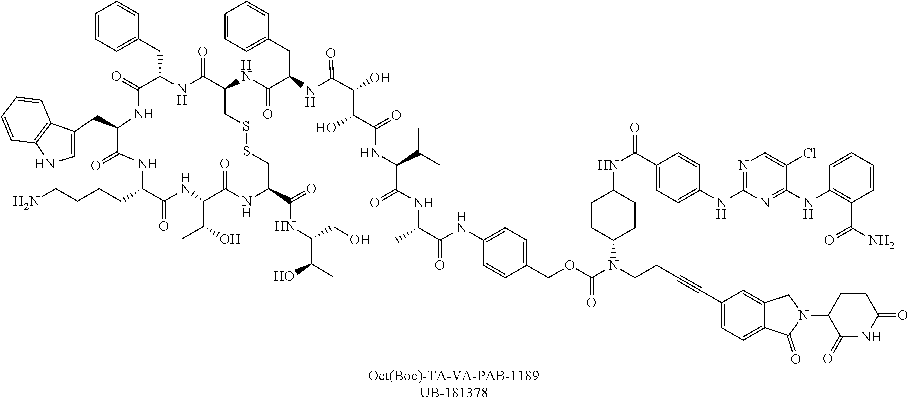

### Step 1: UB-181378b

Compound (2R,3R)-2,3-dihydroxysuccinic acid (670.24 mg, 4.468 mmol) was dissolved in DMF (7.0 ml), and added with EDCI (857.91 mg, 4.468 mmol) and UB-181241a (500 mg, 0.447 mmol). The mixture was stirred for half an hour at room temperature. The reaction solution was spin-dried and purified by reversed-phase C-18 column with H2O:TFA=1000:1):ACN=5%-95% to give white solid (V4747-008, 331 mg, 66% yield). LCMS[M+H]=1151

### Step 2: UB-181378c

Compound UB-181378b (200.00 mg, 0.160 mmol) was dissolved in DMF (7.0 mL), and added with HATU (91.13 mg, 0.240 mmol), UB-181375 (188.17 mg, 0.160 mmol) and DIEA (206.2 mg, 1.599 mmol). The mixture was stirred at room temperature for 1 hour. The reaction solution was spin-dried and purified by reversed-phase C-18 column with (H2O:TFA=1000:1):ACN=5%-95% to give UB-181378c (V4798-015,126 mg, 63% yield) as white solid. LCMS[M+H]=1106

### Step 3: UB-181378

Compound UB-181378c (110 mg, 0.046 mmol) was dissolved in a mixed solution of DCM (2 mL), TFA (1.9 ml), and TIPS (0.1 ml). The mixture was stirred at 0° C. for 10 min. The reaction solution was spin-dried at low temperature, added with MTBE (45 ml), and centrifuged. The supernatant was poured out, and the solid was dissolved in water and acetonitrile, and purified by medium pressure preparative chromatography (0.5% HOAc/H2O) to obtain product, which was lyophilized to obtain white solid UB-181378 (V4827-008, 60.4 mg, 56.8% yield). LCMS[M/2+H]=1155.8 LCMS[M/3+H]=771.1

### Synthesis of Compound UB-181379

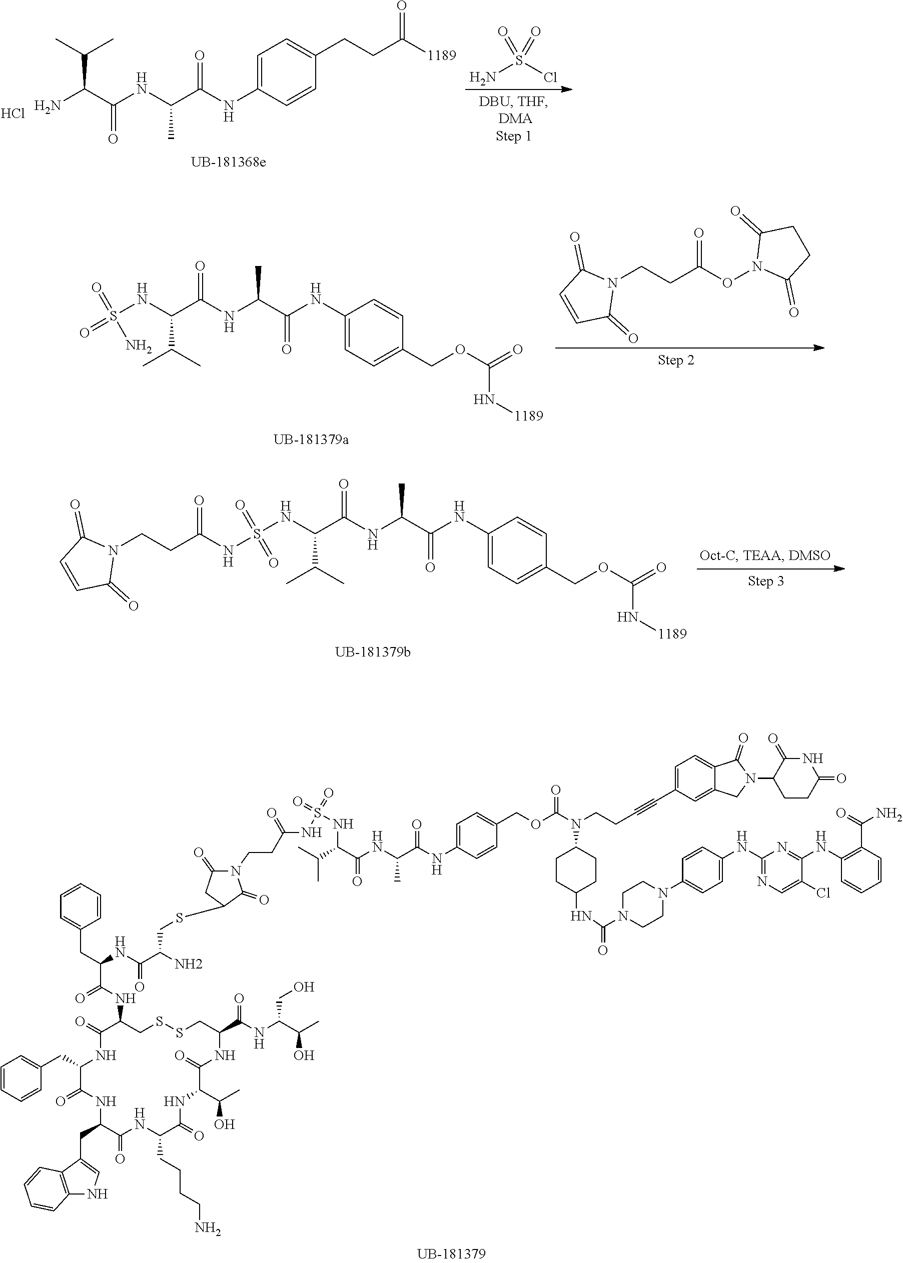

### Step 1: UB-181379a

Compound UB-181368e (600 mg, 0.51 mmol) was dissolved in THF:DMA=1:2 (7.5 ml). To the reaction solution was added DBU (2.3 g, 15.3 mmol), and added dropwise at 0° C. chlorosulfonamide (1.8 g, 15.3 mmol) in THF (2.5 ml). Then the reaction was brought to room temperature and stirred. Complete reaction of UB-181368e was detected by LCMS. The reaction solution was separated directly on reversed-phase column (H2O/CH₃CN=20%˜60% for 20 min). The resulting liquid was lyophilized to give compound UB-18179a (320 mg, 50% yield) as white solid. LCMS: [M+1]⁺=1258.

### Step 2: UB-181379b

Compound UB-181309a (390 mg, 0.32 mmol) was dissolved in DMF (4 mL). After the addition of DIEA (122 mg, 0.96 mmol) and 4-maleimidobutyric-N-hydroxysuccinimidyl ester (168 mg, 0.64 mmol), the reaction was carried on at room temperature for 1 h. When finished, the reaction solution was directly separated by reversed phase column (H2O/CH₃CN=20%˜60% for 20 min), and the resulting liquid was lyophilized to give compound UB-181379c (240 mg, 53% yield) as white solid. LCMS: [M+1]⁺=1409.

### Step 3: UB-181379

Compounds UB-181379b (110 mg, 0.08 mmol) and Oct-C (149 mg, 0.13 mmol) were dissolved in DMSO (2 ml), and 1 mmol/L aqueous TEAA (1 ml) was added dropwise to the reaction solution. Then the reaction was carried on at room temperature for 1 h. When finished, the reaction solution was directly separated by medium pressure preparative chromatography (A: 0.5% HOAc/H2O B: MeCN=35%), and the resulting liquid was lyophilized to give compound UB-181379 (80 mg, 39.5% yield) as white solid. LCMS: [M/3+1]⁺=844. LCMS: [M/2+1]⁺=1265.

### Synthesis of Compound UB-181380, UB-181381, UB-181390

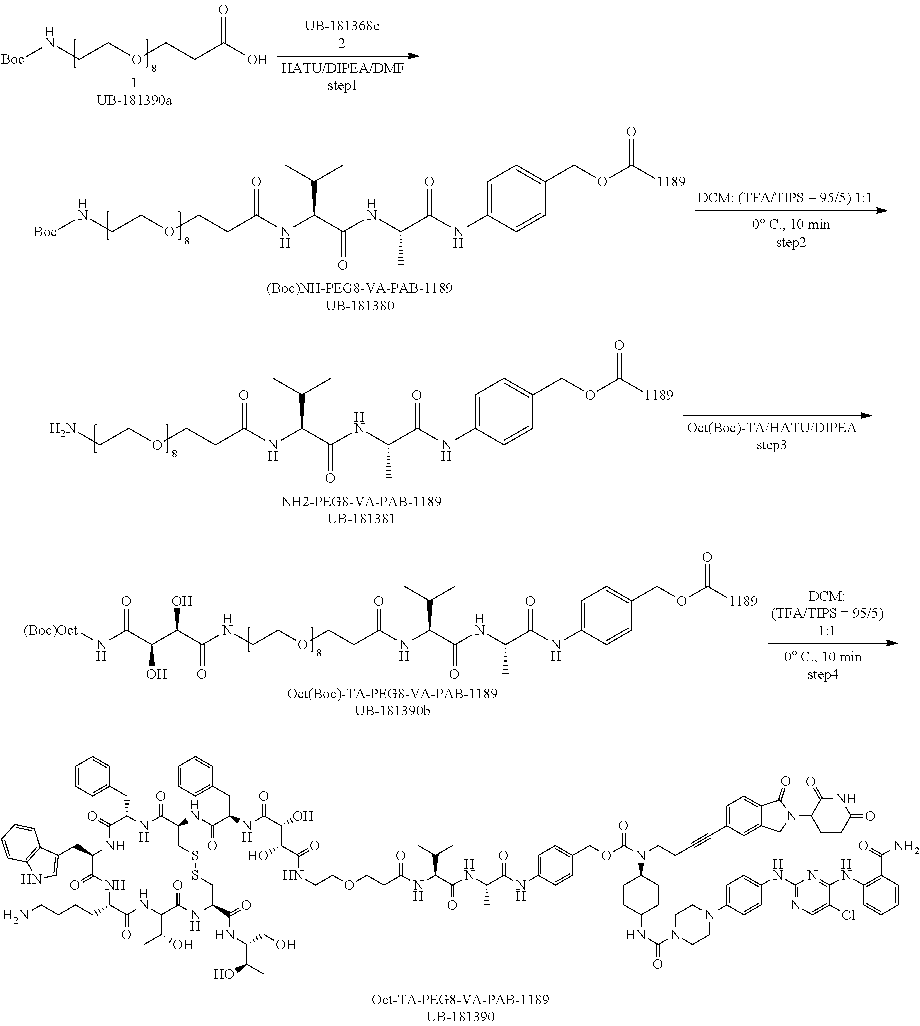

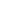

### Step 1: UB-181380

Compound UB-181390a (460 mg, 0.850 mmol), UB-181368e (1 g, 0.850 mmol), HATU (355 mg, 0.935 mmol), and DEA (329 mg, 2.55 mmol) were dissolved in DMF (10 mL), and the mixture was stirred at room temperature for 1 hour. The reaction solution was purified by reversed-phase C-i8 column with MeCN/H2O/0.2% TFA=60/40 to give UB-181380 (V4827-022,920 mg, 64% yield) as yellow solid. LCMS[M/2+H]801.0. ¹H NMR (400 MHz, DMSO) δ 11.85 (s, 1H), 10.99 (s, 1H), 9.89 (d, J=39.2 Hz, 1H), 9.22 (s, 1H), 8.80 (s, 1H), 8.40 (d, J=7.4 Hz, 1H), 8.29 (s, 1H), 8.22-8.14 (m, 2H), 8.00 (d, J=7.9 Hz, 1H), 7.88 (d, J=8.6 Hz, 1H), 7.81 (d, J=7.9 Hz, 1H), 7.76-7.65 (m, 2H), 7.61 (s, 1H), 7.59 (s, 1H), 7.48 (dd, J=12.0, 8.8 Hz, 4H), 7.33 (d, J=8.3 Hz, 2H), 7.09 (t, J=7.5 Hz, 1H), 6.90 (d, J=9.0 Hz, 2H), 6.74 (s, 1H), 5.09 (dd, J=15.5, 7.1 Hz, 3H), 4.46-4.35 (m, 2H), 4.30 (d, J=17.7 Hz, 1H), 4.24-4.10 (m, 1H), 3.79 (s, 2H), 3.60 (t, J=6.5 Hz, 2H), 3.48 (dd, J=12.3, 7.5 Hz, 33H), 3.37 (t, J=6.1 Hz, 2H), 3.10-3.00 (m, 6H), 2.96-2.84 (m, 1H), 2.69 (dd, J=6.9, 5.1 Hz, 2H), 2.60 (s, 2H), 2.48-2.28 (m, 4H), 1.96 (dd, J=13.6, 6.8 Hz, 2H), 1.86 (t, J=17.5 Hz, 4H), 1.50 (s, 4H), 1.37 (s, 9H), 1.31 (d, J=7.1 Hz, 3H), 0.91-0.79 (m, 6H).

### Step 2: UB-181381

DCM (3 mL), TFA (2.85 ml) and TIPS (0.15 ml) were mixed well. Compound UB-181380 (500 mg, 0.29 mmol) was dissolved in the above solution and stirred at 0° C. for 10 minutes. The reaction solution was spin-dried at low temperature, added with MTBE (45 ml), and centrifuged. The supernatant was poured out. The solid was dissolved with water and acetonitrile and purified by C-18 flash column with 0.5% HOAc/H2O to give UB-181381 (V4827-025, 280 mg, 60% yield) as white solid. LCMS[M/2+H]=800.8. ¹H NMR (400 MHz, DMSO) δ 11.85 (s, 1H), 9.95 (s, 1H), 9.23 (s, 1H), 8.79 (s, 1H), 8.28 (s, 1H), 8.18 (dd, J=10.6, 4.0 Hz, 1H), 7.89 (d, J=8.7 Hz, 1H), 7.81 (d, J=7.7 Hz, 1H), 7.76-7.65 (m, 2H), 7.60 (d, J=7.3 Hz, 2H), 7.52-7.43 (m, 4H), 7.33 (d, J=8.4 Hz, 2H), 7.09 (t, J=7.6 Hz, 1H), 6.90 (d, J=9.1 Hz, 2H), 6.12 (d, J=5.5 Hz, 1H), 5.09 (dd, J=15.1, 6.8 Hz, 3H), 4.45-4.35 (m, 2H), 4.30 (d, J=17.7 Hz, 1H), 4.25-4.09 (m, 1H), 3.79 (s, 2H), 3.60 (t, J=6.6 Hz, 2H), 3.54 (d, J=5.7 Hz, 6H), 3.50 (ddd, J=10.4, 6.5, 3.4 Hz, 29H), 3.04 (s, 4H), 2.94-2.84 (m, 3H), 2.75-2.67 (m, 2H), 2.62-2.53 (m, 2H), 2.48-2.28 (m, 4H), 1.96 (dd, J=13.4, 6.7 Hz, 2H), 1.83 (d, J=10.9 Hz, 4H), 1.50 (s, 4H), 1.33-1.27 (m, 3H), 0.90-0.79 (m, 6H).

### Step 3: UB-181390b

Compounds Oct (Boc)-TA (300 mg, 0.238 mmol), and HATU (90 mg, 0.238 mmol) were dissolved in DMF (3 mL), and the mixture was stirred at room temperature for 10 min. UB-181381 (190 mg, 0.119 mmol) dissolved in DMF (2 mL) and DIEA (77 mg, 0.595 mmol) and then added to the above reaction solution. The reaction solution was stirred for 1 hour at room temperature. The reaction solution was purified by reversed-phase C-18 column with 0.1% TFA/H2O/MeCN=45/55 to give UB-181390b (V4827-029, 160 mg, 47% yield) as yellow solid. LCMS[M/3+H]=912 LCMS[M/2+H]=1418 Step 4: UB-181390 DCM (3 mL), TFA (2.85 ml) and TIPS (0.15 ml) were mixed well. Compound UB-181390b (160 mg, 0.056 mmol) was dissolved in the above solution and stirred at 0° C. for 10 minutes. The reaction solution was spin-dried at low temperature, added with MTBE (45 ml), and centrifuged. The supernatant was poured out. The solid was dissolved with water and acetonitrile, and purified by C-18 flash with 0.5% HOAc/H2O and medium pressure preparative chromatography with A:0.5% HOAc/H2O B: 80% MeCN/20% H2O/0.5% HOAc C: 0.1 mmol/L NH4OAc/H2O S1:20%-20% 2CV C, 20%-20% 2CV A, S1 (A):S2 (B)=20%-90% 2CV to give UB-181390 (V4827-032, 23.3 mg, yield 15%) as white solid. LCMS[M/3+H]=912.4 LCMS[M/2+H]=1369.0

### Synthesis of Compound UB-181391

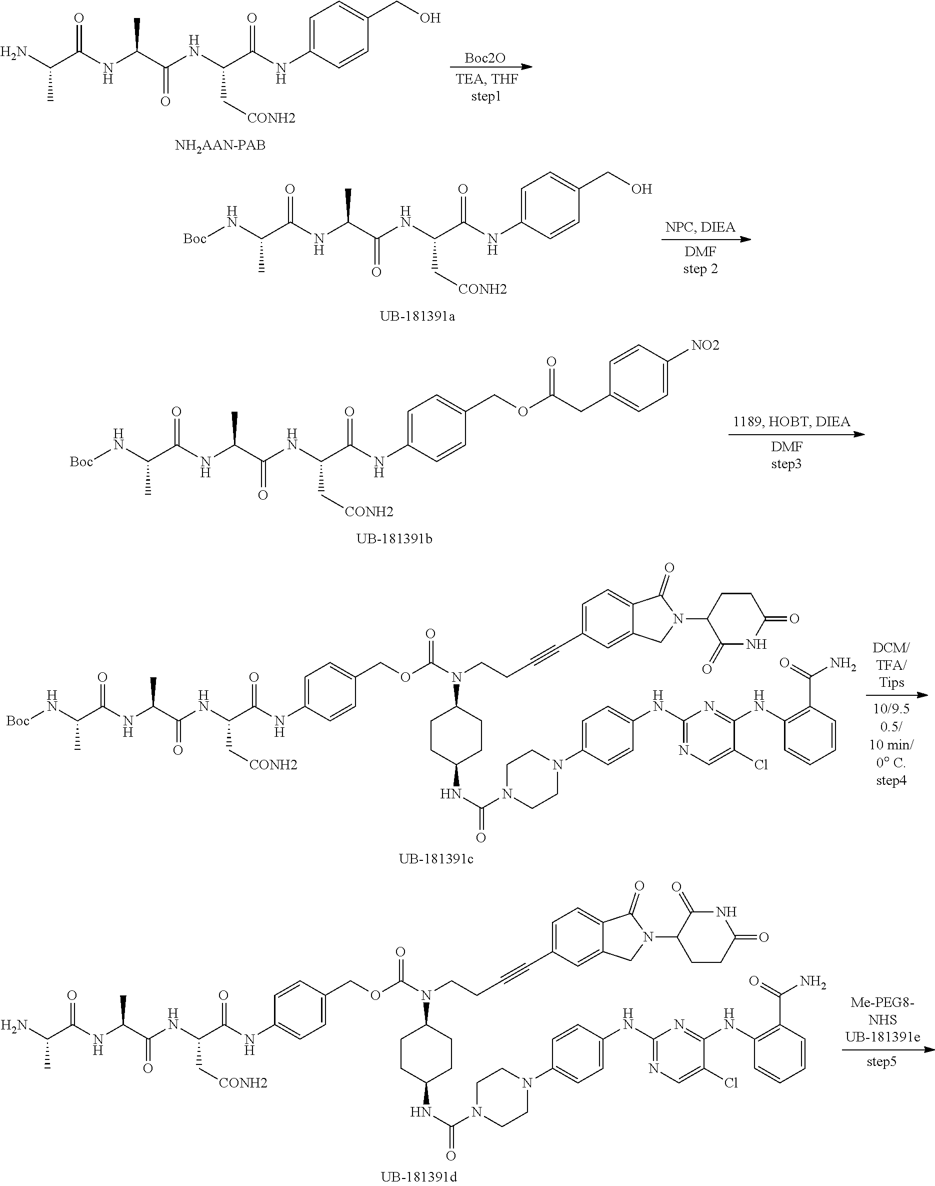

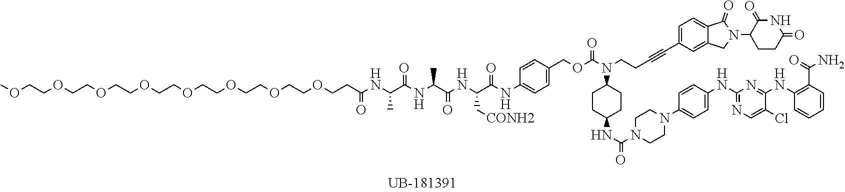 [SMILES: COCCOCCOCCOCCOCCOCCOCCOCCC(=O)N[C@@H](C)C(=O)N[C@@H](C)C(=O)N[C@@H](CC(N)=O)C(=O)Nc1ccc(COC(=O)N(CCC#Cc2ccc3c(c2)CN(C2CCC(=O)NC2=O)C3=O)[C@H]2CC[C@@H](NC(=O)N3CCN(c4ccc(Nc5ncc(Cl)c(Nc6ccccc6C(N)=O)n5)cc4)CC3)CC2)cc1]

### Step 1: UB-181391a

Compound NH₂-AAN-PAB (5 g, 13.2 mmol), (Boc)₂O (5.7 g, 26.4 mmol), and DIEA (6.8 g, 52.8 mmol) were dissolved in THF (50 mL), and then the mixture was reacted at room temperature for 30 min. The reaction solution was concentrated and separated by column chromatography (methanol/dichloromethane=1/10) to obtain target product UB-181391a (1.8 g, yield 28%) as white solid. LCMS [M+H]⁺=480.4.

### Step 2: UB-181391b

Compound UB-181391a (50 mg, 0.104 mmol) was dissolved in DMF (3 mL), then bis(p-nitrophenyl)carbonate (66 mg, 0.209 mmol) and DIEA (40 mg, 0.313 mmol) were added, and the mixture was reacted at room temperature for 4 hours. The reaction solution was concentrated and separated by column chromatography (dichloromethane/methanol=10/1) to obtain target product UB-181391b (35 mg, yield 52%) as white solid. LCMS [M+H]⁺=645.4

### Step 3: UB-181391c

Compound UB-181391b (2.06 g, 3.2 mmol) was dissolved in DMF (20 mL), then UB-181189 (2.7 g, 3.2 mmol), HOBT (1.4 g, 6.4 mmol) and DIEA (1.2 g, 9.6 mmol) were added, and the mixture was reacted at room temperature for 1 hour. The reaction solution was concentrated and separated by column chromatography (dichloromethane/methanol=10/1) to obtain target product UB-181391c (1.6 g, yield 37%) as white solid. LCMS [M/2+H]⁺=682.4

### Step 4: UB-181391d

Compound UB-181391c (600 mg, 0.44 mmol) was dissolved in DCM/TFA/TIPS=10/9.5/0.5 (6 mL) under ice bath and the mixture was reacted for 10 min at room temperature. The reaction solution was concentrated at low temperature and then the residue was pulped with dichloromethane/diethyl ether=1/10 to obtain target product UB-181391d (600 mg, yield 100%) as yellow solid. LCMS [M+H]⁺=1266.4

### Step 5: UB-181391

Compound UB-181391d (100 mg, 0.08 mmol) was dissolved in DMF (3 mL), then DIEA (20 mg, 0.16 mmol) and UB-181391e (40 mg, 0.08 mmol) were added, and the mixture was reacted at room temperature for 1 hour. The reaction solution was subjected to preparative chromatography to obtain target product UB-181391 (51.6 mg, yield 39%) as white solid. LCMS [M/2+H]⁺=829.4. ¹H NMR (400 MHz, DMSO-d₆) δ 11.83 (s, 1H), 10.98 (s, 1H), 9.67 (s, 1H), 9.21 (s, 1H), 8.78 (s, 1H), 8.28 (s, 1H), 8.19-8.05 (m, 4H), 7.80 (dd, J=7.9, 1.6 Hz, 1H), 7.75-7.56 (m, 5H), 7.47 (q, J=9.1 Hz, 4H), 7.40 (s, 1H), 7.32 (d, J=8.2 Hz, 2H), 7.09 (t, J=7.6 Hz, 1H), 6.98-6.83 (m, 3H), 6.11 (d, J=5.8 Hz, 1H), 5.11-4.94 (m, 3H), 4.61 (q, J=6.8 Hz, 1H), 4.47-4.11 (m, 4H), 3.78 (s, 2H), 3.59 (t, J=6.6 Hz, 2H), 3.52-3.44 (m, 31H), 3.23 (s, 3H), 3.03 (t, J=4.9 Hz, 4H), 2.94-2.81 (m, 1H), 2.69 (d, J=8.2 Hz, 2H), 2.57 (dd, J=6.6, 3.3 Hz, 2H), 2.42-2.28 (m, 3H), 2.05-1.93 (m, 2H), 1.82 (d, J=13.5 Hz, 5H), 1.49 (s, 4H), 1.22 (dd, J=7.1, 4.5 Hz, 8H).

### Synthesis of Compound UB-181387& UB-181388& UB-181389& UB-181393

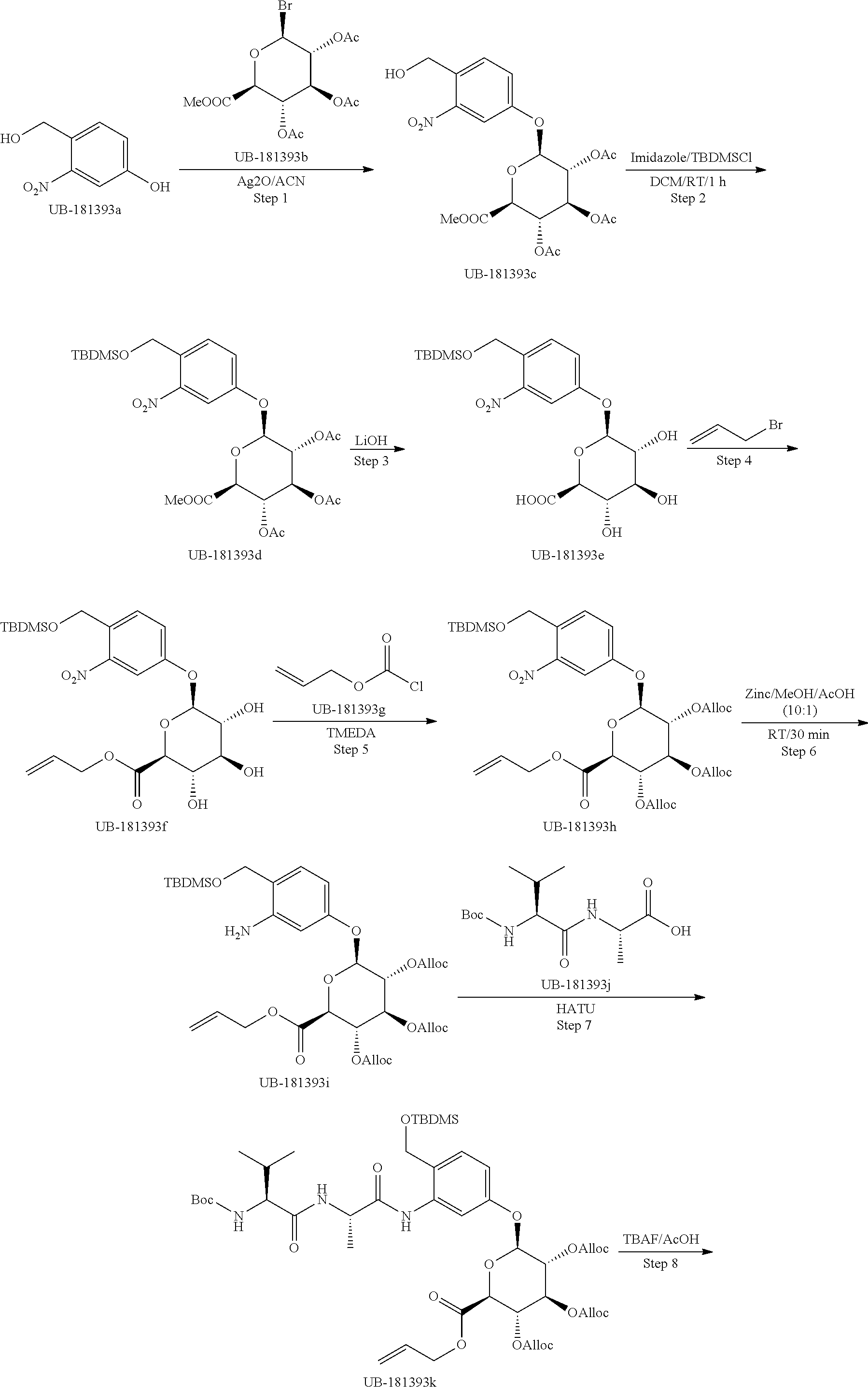

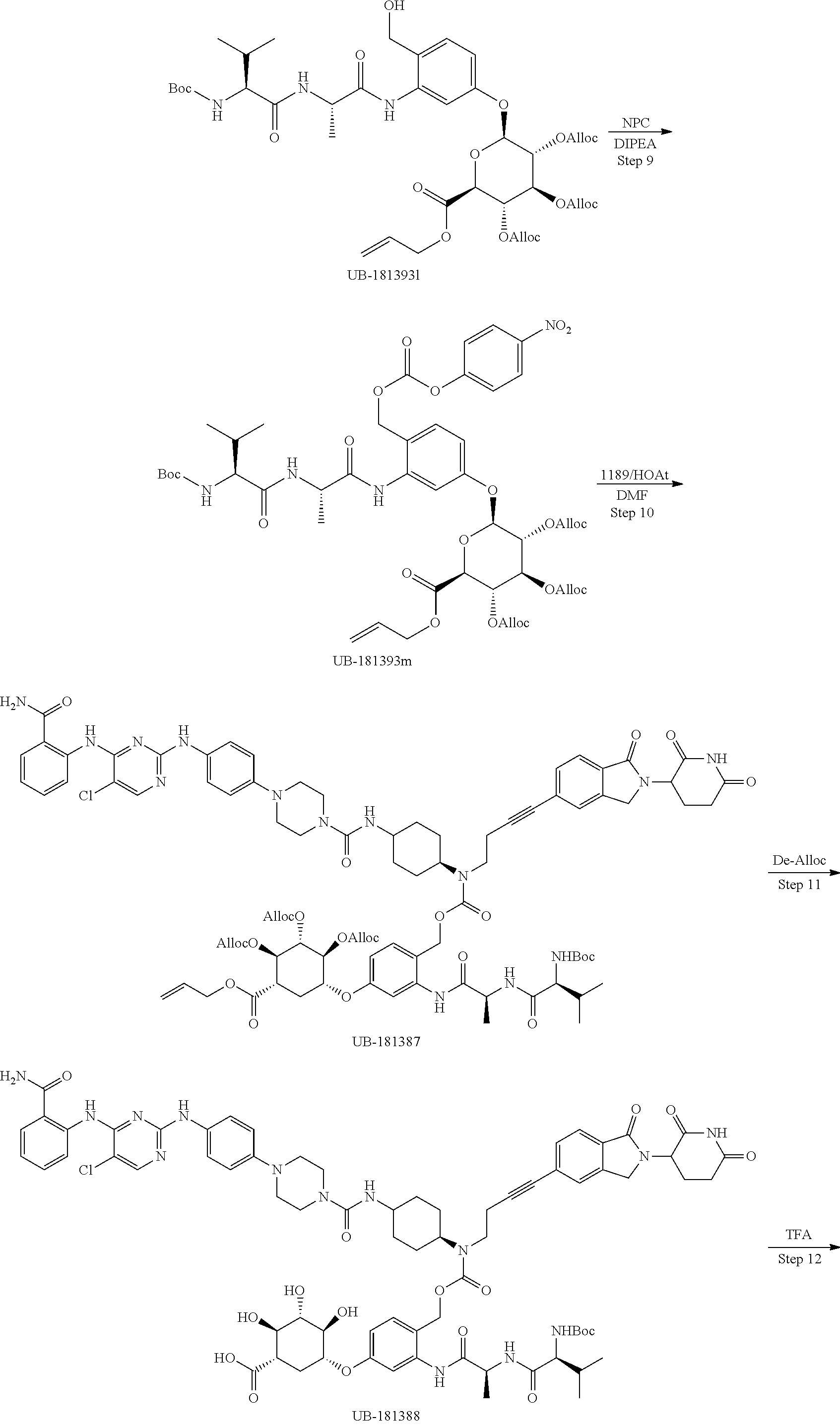

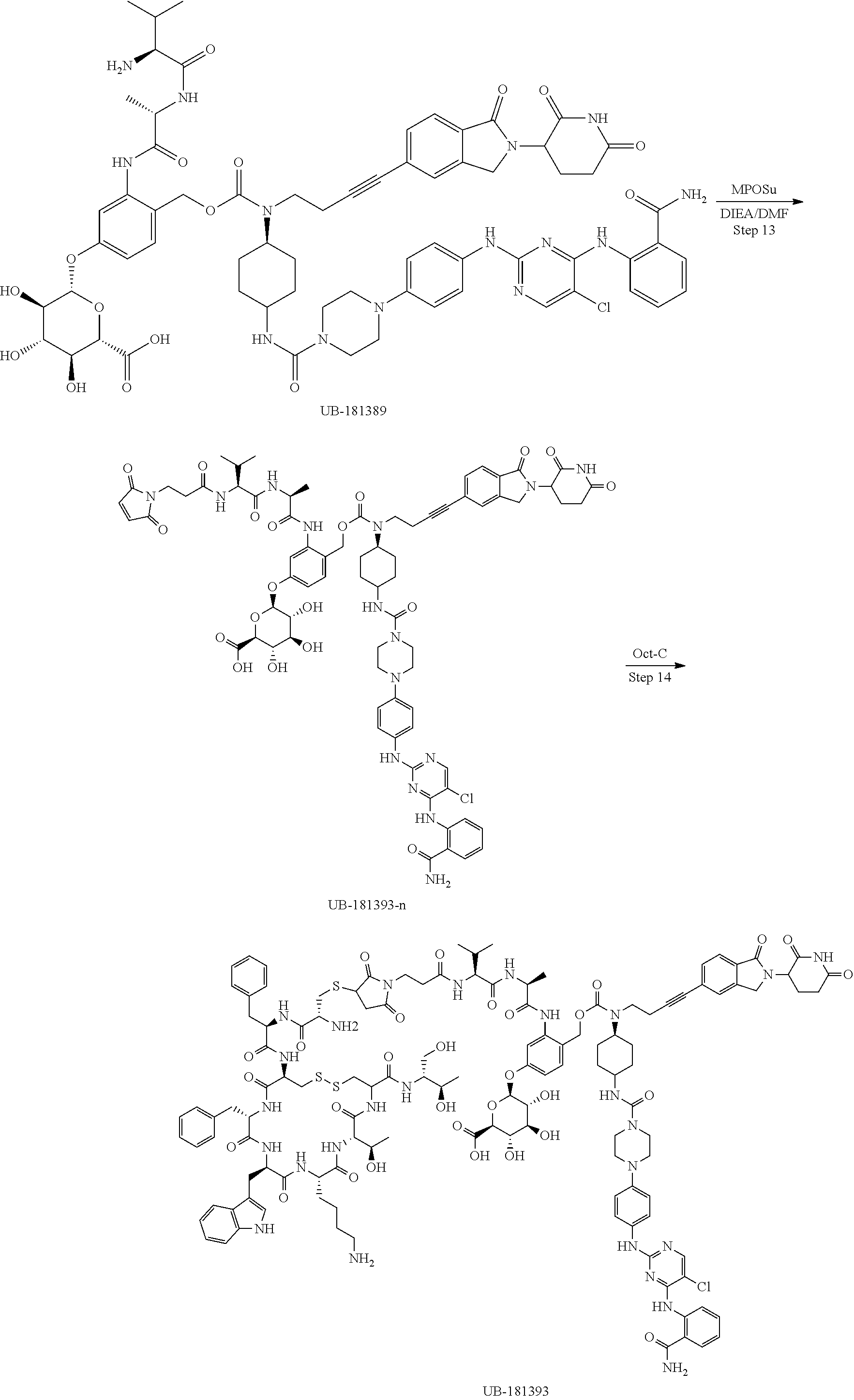

### Step 1: UB-181393c

UB-181393a (15 g, 89.8 mmol) was dissolved in anhydrous acetonitrile (600 mL) and UB-181393b (35.47, 89.8 mmol) and Ag₂O (61.8 g, 269.4 mmol) were added successively. The reaction was stirred for 18 h under argon protection and protection from light. After completion of the reaction, the solid was removed by filtration, and the reaction solution was washed with acetonitrile and concentrated in vacuum. The crude was purified by silica gel column chromatography (PE/EtOAc, from 70/30 to 50/50) to obtain UB-181393c (29 g, 93% yield) as yellow solid. Rf=0.28 (PE/EtOAc:50/50).

### Step 2: UB-181393d

UB-181393c (12 g, 24.8 mmol) and imidazole (3.4 g, 49.7 mmol) were dissolved in DCM (500 mL), added with TBDMSCl (7.5 g, 49.7 mmol) and the mixture was reacted for 1 hour at room temperature. The reaction was quenched by adding saturated aqueous NaHCO₃ and extracted with DCM. The combined organic layer was dried over MgSO4, filtered, and concentrated under reduced pressure. The residue was purified by silica gel column chromatography (PE/EtOAc, from 80/20 to 60/40) to obtain UB-181393d (14 g, 93% yield) as white solid. LCMS [M+1]+=600.5.

### Step 3: UB-181393e

To a solution of UB-181393d (18 g, 30.1 mmol) in MeOH/THF was added LiOH/H₂O (1.9 g, 45.1 mmol) at 0° C. The mixture was reacted at 0° C. for 1 hour. To the reaction solution was added AcOH (pH=6). The mixture was concentrated and purified by silica gel column chromatography (DCM/MeOH=0 to 25%) to obtain UB-181393e (12.1 g, 88% yield). ¹H NMR (400 MHz, DMSO) δ 7.68-7.55 (m, 2H), 7.44-7.32 (m, 1H), 5.14 (d, J=3.3 Hz, 1H), 4.71 (s, 2H), 3.95-3.82 (m, 1H), 3.41-3.25 (m, 2H), 2.05-1.72 (m, 1H), 0.76 (s, 9H), 0.00 (s, 6H).LCMS[M+H]+=460.3.

### Step 4: UB-181393f

UB-181393e (200 mg, 0.4 mmol), and DBU (132.5 mg, 0.9 mmol) were dissolved in DMF (2 mL), and the mixture was stirred at room temperature for 30 min and added with allyl bromide (105.4 mg, 0.9 mmol). The mixture was reacted at 50° C. for 16 hours. The reaction solution was concentrated under reduced pressure, and purified by silica gel column chromatography (MeOH/DCM=1/10) to obtain UB-181393f (100 mg, 46% yield). ¹H NMR (400 MHz, CDCl₃) δ 7.69-7.26 (m, 2H), 7.23 (dd, J=8.7, 2.5 Hz, 1H), 5.77 (ddt, J=16.1, 10.5, 5.7 Hz, 1H), 5.19 (ddd, J=17.2, 2.8, 1.4 Hz, 1H), 5.09 (dt, J=15.0, 7.5 Hz, 1H), 4.96-4.83 (m, 1H), 4.73 (d, J=4.4 Hz, 2H), 4.65-4.46 (m, 2H), 4.01 (dd, J=18.7, 8.3 Hz, 1H), 3.85 (d, J=8.7 Hz, 1H), 3.75 (dd, J=24.0, 14.9 Hz, 2H), 2.85 (s, 1H), 2.85 (s, 1H), 0.83 (s, 9H), 0.00 (s, 6H). LCMS[M+H]+=500.3.

### Step 5: UB-181393h

UB-181393f (740 mg, 1.5 mmol) and TMEDA (1.1 ml, 7.5 mmol) were dissolved in dried DCM (10 mL), added with UB-181393g (2.7 ml, 44.5 mmol) and the mixture was reacted for 1 hour at room temperature. Then the reaction solution was filtered. The filtrate was concentrated to obtain crude product, which was purified by silica gel column chromatography (PE/EA: 80/20) to obtain UB-181393h (1 g, 92% yield) as yellow oil. LC-MS:[M+H]+=752.6.

### Step 6: UB-181393i

UB-181393h (3.78 g, 5 mmol) was dissolved in THF/AcOH (20 mL/4 ml, 5:1 v/v) and active zinc powder (16.4 g, 250 mmol) was added at one time. The mixture was reacted for 1 hour under argon atmosphere. The crude was purified by reversed phase column (H2O:acetonitrile=0-81%). The H2O/ACN solution was extracted twice with EA (40 ml). EA was concentrated to give UB-181393i (2.7 g, 75% yield). LC-MS:[M+H]+=722.5.

### Step 7: UB-181393k

UB-181393i (2.6 g, 3.6 mmol), UB-181393j (1.04 g, 3.6 mmol), HATU (2.05 g, 5.4 mmol) and DIPEA (930 mg, 7.2 mmol) were dissolved in dry THF (50 ml) and the mixture was stirred for 0.5 hour at room temperature. The reaction solution was concentrated and purified by silica gel column chromatography (DCM/MeOH: 10/1) to obtain UB-181393k (4.8 g, 100% yield) as yellow oil. LC-MS:[M+H]+=993.8.

### Step 8: UB-181393l

HF/pyridine 70% (17 ml) was added dropwise to a solution of UB-181393k (4.5 g, 4.5 mmol) in anhydrous THF (50 ml) at 0° C., and the mixture was reacted for 2 hours at room temperature. Then the reaction was quenched with saturated NaHCO3 solution (30 ml) and extracted with EtOAc (3×40 ml). The combined organic layer was dried over MgSO4, filtered, and concentrated under reduced pressure. The residue was purified by silica gel column chromatography (eluent DCM/MeOH: 15/1) to obtain UB-181393l (2.44 g, 63% yield) as colorless oil. LC-MS:[M+H]+=878.6.

### Step 9: UB-181393m

UB-181393l (2.4 g, 2.7 mmol) and PNC (2.5 g, 8.2 mmol) were dissolved in anhydrous DMF (10 100 mL), added with DIEA (1.06 g, 8.2 mmol) and the mixture was reacted for 1 hour at room temperature. The reaction was poured into H2O and extracted with EtOAc (3×40 ml). The crude product was purified by silica gel column chromatography (eluent DCM/MeOH: 15/1) to obtain UB-181393m (2.55 g, 90% yield) as white solid. LC-MS:[M+H]+=1043.9.

### Step 10: UB-181393

1189 (1.4 g, 1.6 mmol) and DIEA (6.7 mg, 4.6 mmol) were dissolved in DMF (5 ml). UB-181393m (2.45 g, 2.3 mmol) and HOAt (320 mg, 2.3 mmol) were added. The mixture was reacted at 30° C. for 4 hours. The reaction was poured into H2O and extracted with EtOAc (3×40 ml). The crude product was purified by silica gel column chromatography (DCM/MeOH: 15/1) to obtain UB-181387 (1.05 g, 25% yield) as white solid. LC-MS:[1/2M+H]+=881.3.

### Step 11: UB-181388

UB-181387 (400 mg, 0.2 mmol) was dissolved in anhydrous THF (20 ml), morpholine (200 mg, 2.0 mmol, 10 eq.) was added dropwise and the mixture was reacted under argon for 10 min, followed by the addition of Pd (PPh₃)₄ (52 mg, 0.04 mmol). The solution was stirred for 30 minutes at 0° C. The crude product was purified by reversed-phase column (H2O:acetonitrile=0-100%) to afford UB-181388 (150 mg, 45% yield) as white solid. LC-MS:[M+H]+=1469.2

### Step 12: UB-181389

UB-181388 (50 mg, 0.035 mmol) was dissolved in TFA/DCM=3/7 (2 ml) and stirred for 2 hours. Isopropyl ether (50 ml) was added to the mixture, and the solution was stirred for 10 minutes. The mixture was centrifuged. The solid was purified by reversed-phase column (H2O:acetonitrile=0-100%) to afford UB-181389 (30 mg, 70% yield) as white solid. LC-MS:[M+H]+=1369.2.

### Step 13: UB-181393n

UB-181389 (220 mg, 0.16 mmol) and MPOSu (86 mg, 0.32 mmol) were successively dissolved in DMF (5 mL), and added with DIEA (41 mg, 0.32 mmol). The mixture was reacted for 30 min. The mixture was purified by reversed-phase column with (H2O (0.5% AcOH):acetonitrile=40%) to give UB-181393n (105 mg, 44% yield) as white solid. LC-MS:[M+H]+=1522.2.

### Step 14: UB-181393

UB-181393n (100 mg, 0.06 mmol) and Oct-C (150 mg, 0.12 mmol) were successively dissolved in DMF (1 ml). The mixture was reacted at 30° C. for 1 hour. The mixture was purified by a reversed-phase column with (H2O (0.05% AcOH): acetonitrile=0-100%) to give UB-181393 (20 mg, 16% yield) as white solid. LC-MS:[1/3M+H]+=881.4.

### Synthesis of Compound UB-181394

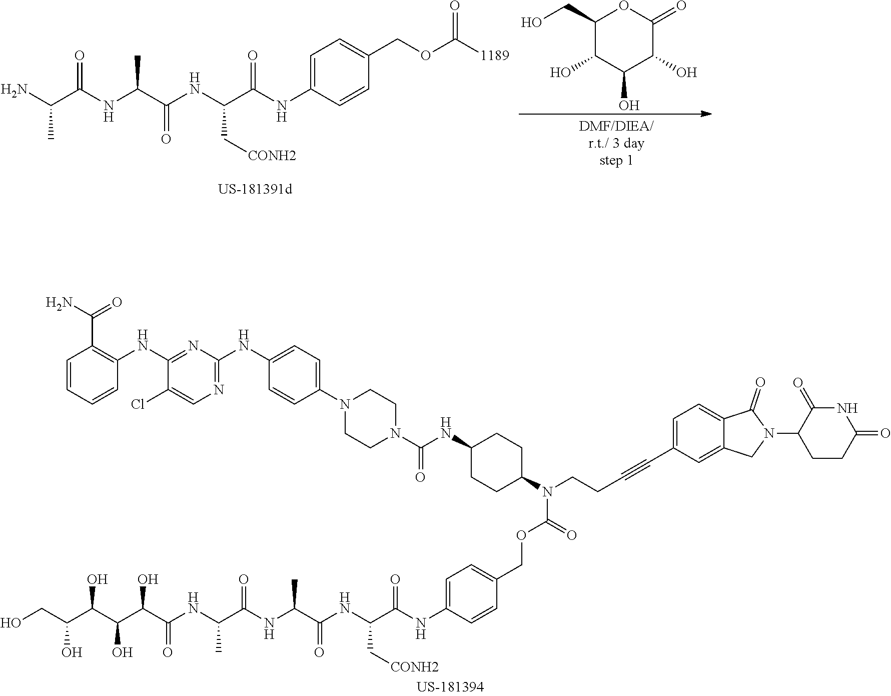

### Step 1: UB-181394

Compound UB-181391d (170 mg, 0.13 mmol) was dissolved in DMF (5 mL), and then added with TEA (27 mg, 0.27 mmol) and UB-181394a (120 mg, 0.67 mmol), and the mixture was reacted at room temperature for 3 days. The reaction solution was concentrated, and then subjected to preparative chromatography to obtain target product UB-181394 (41.4 mg, yield 21%) as white solid. LCMS [M/2+H]+=721.4. ¹H NMR (400 MHz, DMSO-d₆) δ 11.84 (s, 1H), 10.98 (s, 1H), 9.66 (s, 1H), 9.22 (s, 1H), 8.79 (s, 1H), 8.28 (s, 1H), 8.21 (d, J=6.6 Hz, 1H), 8.16 (s, 1H), 8.05 (d, J=7.6 Hz, 1H), 7.85 (d, J=7.4 Hz, 1H), 7.80 (dd, J=8.0, 1.6 Hz, 1H), 7.77-7.54 (m, 5H), 7.47 (q, J=9.7, 9.3 Hz, 4H), 7.40 (s, 1H), 7.32 (d, J=8.1 Hz, 2H), 7.16-7.02 (m, 1H), 6.95 (s, 1H), 6.93-6.81 (m, 2H), 6.12 (d, J=5.8 Hz, 1H), 5.65 (d, J=4.9 Hz, 1H), 5.20-4.96 (m, 3H), 4.67-4.50 (m, 4H), 4.45-4.16 (m, 5H), 4.07 (dd, J=4.9, 3.5 Hz, 1H), 3.95 (dt, J=6.5, 2.9 Hz, 1H), 3.79 (s, 3H), 3.64-3.36 (m, 9H), 3.03 (s, 4H), 2.99-2.79 (m, 3H), 2.70 (s, 2H), 2.65-2.52 (m, 3H), 2.35 (dd, J=13.2, 4.4 Hz, 1H), 2.08-1.90 (m, 1H), 1.82 (d, J=12.1 Hz, 5H), 1.49 (s, 5H), 1.36-1.19 (m, 7H), 1.12 (t, J=7.2 Hz, 2H).

### Synthesis of Compound UB-181396

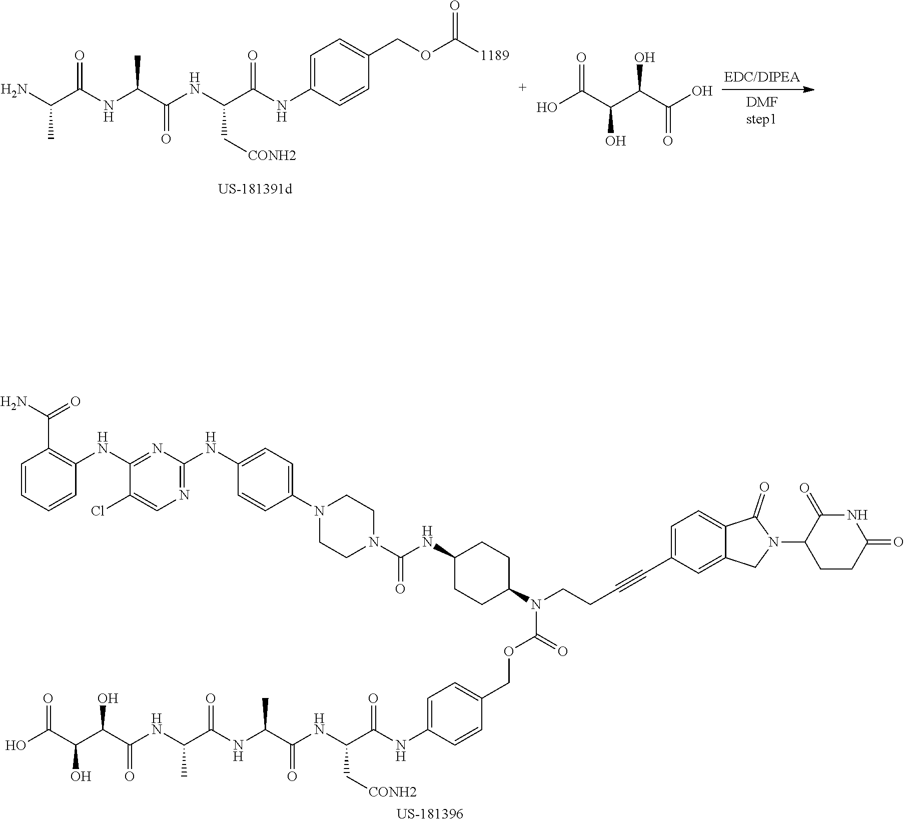

### Step 1: UB-181396

Compound UB-181391d (450 mg, 0.36 mmol), L-tartaric acid (1.07 g, 7.13 mmol), EDCI (1.36 g, 7.13 mmol), and DIEA (919 mg, 7.13 mmol) were dissolved in DMF (10 mL), and the reaction solution was reacted at room temperature for 1 hour. The reaction solution was directly separated by reverse phase column (pure water/acetonitrile), and the resulting liquid was lyophilized to obtain compound UB-181396 (48 mg, yield 10%) as white solid. LCMS[M/2+H]=698.6

The exemplary TEDs, ACTEDs, and intermediates of the present invention are shown in the tables below:

TABLE Al
Exemplary TED Compounds

| TED | Compound | |
|---|---|---|
| No. | No. | Structure and Characterization |
| PT01 | UB-180961 | 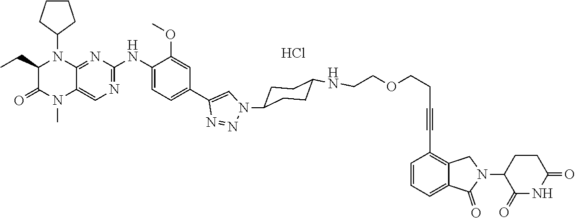 [SMILES: CC[C@@H]1C(=O)N(C)c2cnc(Nc3ccc(-c4cn(C5CCC(NCCOCCC#Cc6cccc7c6CN(C6CCC(=O)NC6=O)C7=O)CC5)nn4)cc3OC)nc2N1C1CCCC1 ; Cl] |
| PT02 | UB-181103 | 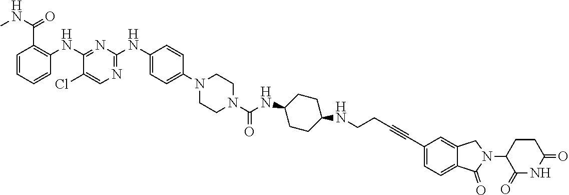 [SMILES: CNC(=O)c1ccccc1Nc1nc(Nc2ccc(N3CCN(C(=O)N[C@H]4CC[C@@H](NCCC#Cc5ccc6c(c5)CN(C5CCC(=O)NC5=O)C6=O)CC4)CC3)cc2)ncc1Cl] |
| PT03 | UB-181189 | 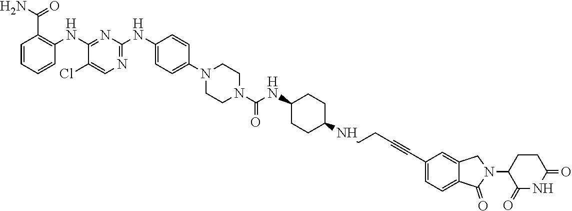 [SMILES: NC(=O)c1ccccc1Nc1nc(Nc2ccc(N3CCN(C(=O)N[C@H]4CC[C@@H](NCCC#Cc5ccc6c(c5)CN(C5CCC(=O)NC5=O)C6=O)CC4)CC3)cc2)ncc1Cl] |
| PT04 | UB-180937 | 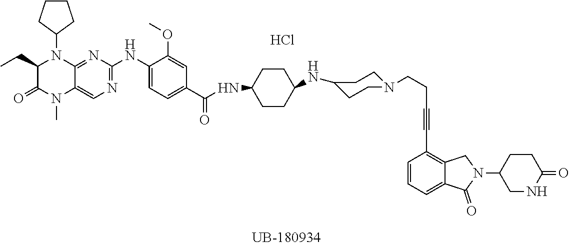 [SMILES: CC[C@@H]1C(=O)N(C)c2cnc(Nc3ccc(C(=O)N[C@H]4CC[C@@H](NC5CCN(CCC#Cc6cccc7c6CN(C6CCC(=O)NC6)C7=O)CC5)CC4)cc3OC)nc2N1C1CCCC1 ; Cl] |
| PT05 | UB-180941 | 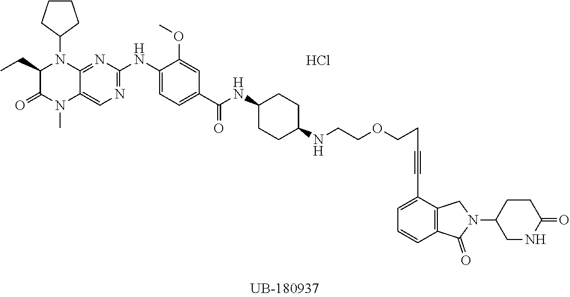 [SMILES: CC[C@@H]1C(=O)N(C)c2cnc(Nc3ccc(C(=O)N[C@H]4CC[C@@H](NCCOCCC#Cc5cccc6c5CN(C5CCC(=O)NC5)C6=O)CC4)cc3OC)nc2N1C1CCCC1 ; Cl] |

TABLE A2
Exemplary TED Compounds

| TED | Compound | |
|---|---|---|
| No. | No. | Structure and Characterization |
| NT01 | UB-181235 | 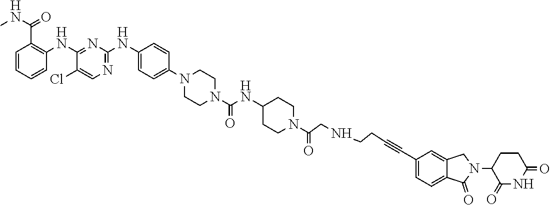 [SMILES: CNC(=O)c1ccccc1Nc1nc(Nc2ccc(N3CCN(C(=O)NC4CCN(C(=O)CNCCC#Cc5ccc6c(c5)CN(C5CCC(=O)NC5=O)C6=O)CC4)CC3)cc2)ncc1Cl] |
| | | ¹H NMR(400 MHz, DMSO-d₆) δ 11.75(s, 1H), 11.00(s, 1H), 9.57(s,1H), 9.01-8.77(m, 2H), |
| | | 8.71(d, J = 7.4 Hz, 2H), 8.22(s 1H), 7.82-7.68(m, 3H), 7.68-7.51(m, 3H), 7.47(t, J = 7.9 Hz, |
| | | 1H), 7.17(dd, J = 7.5, 5.4 Hz, 3H), 5.93(s, 1H), 5.11(dd, J = 13.3, 5.1 Hz, 1H), 4.46(d, J = |
| | | 7.7 Hz, 1H), 4.33(d, J = 7.7 Hz, 1H),3.68(s, 1H), 3.19(d, J = 7.7 Hz, 3H), 3.21-2.90(m, 3H), |
| | | 2.90-2.49(m, 7H), 2.37(ddd, J = 6.2, 10.5, 7.3 Hz, 2H), 2.04-1.99(m, 2H), 1.83(dd, J = 2.7, |
| | | 9.1 Hz, 1H), 1.65(d, J = 4.4 Hz, 8H), 1.57-1.27(m, 4H). LCMS [M + H]⁺ = 915.6 |
| NT02 | UB-181236 | 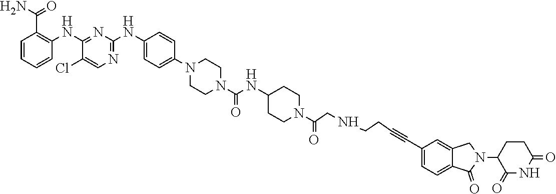 [SMILES: NC(=O)c1ccccc1Nc1nc(Nc2ccc(N3CCN(C(=O)NC4CCN(C(=O)CNCCC#Cc5ccc6c(c5)CN(C5CCC(=O)NC5=O)C6=O)CC4)CC3)cc2)ncc1Cl] |
| | | ¹H NMR(400 MHz, DMSO-d₆) δ 11.62(s, 1H), 11.00(s, 1H), 10.24-10.19(m, 1H), 9.24(s, |
| | | 1H), 8.80(s, 1H), 8.16(s, 1H), 7.82-7.65(m, 3H), 7.59(t, J = 13.4 Hz, 1H), 7.48(dd, J = |
| | | 14.1, 8.3 Hz, 3H), 6.92(d, J = 9.0 Hz, 2H), 6.48(s, 1H), 5.12(dd, J = 13.2, 5.0 Hz, 1H), |
| | | 4.46(d, J = 17.7 Hz, 2H), 4.39-4.28(m, 1H), 4.28-3.86(m, 1H), 3.69(d, J = 13.1 Hz, 2H), |
| | | 3.23(d, J = 8.2 Hz, 4H), 3.18-2.92(m, 7H), 2.92-2.85(m, 3H), 2.85-2.47(m, 6H), 1.99(dd, |
| | | J = 12.3, 10.1 Hz, 1H), 1.81(s, 2H), 1.20(t, J = 7.3 Hz, 3H). LCMS [M + H]⁺ = 900.7 |
| NT03 | UB-181239 | 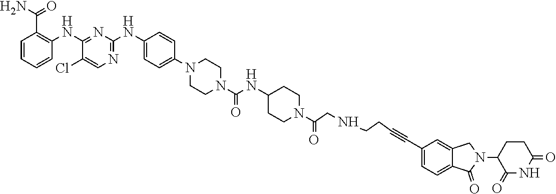 [SMILES: NC(=O)c1ccccc1Nc1nc(Nc2ccc(N3CCN(C(=O)NC4CCN(C(=O)CNCCC#Cc5ccc6c(c5)CN(C5CCC(=O)NC5=O)C6=O)CC4)CC3)cc2)ncc1Cl] |
| | | ¹H NMR(400 MHz, DMSO-d₆) δ 11.87(s, 1H), 11.00(s, 1H), 10.28(s, 2H), 9.24(s, 1H), |
| | | 8.80(s, 1H), 8.46(s, 1H),8.16(s, 1H), 7.87-7.66(m, 5H), 7.48(t, J = 10.2 Hz, 3H), 7.09 (s, |
| | | 1H), 6.93(d, J = 9.1 Hz, 2H), 6.58-6.43(m, 1H), 5.12(dd, J = 13.2, 5.0 Hz, 1H), 4.39-4.23 |
| | | (m, 3H), 4.07(dd, J = 3.48, 1.66 Hz, 2H), 3.73-3.49(m, 4H), 3.46(s, 2H), 3.05(q, J = 7.3 |
| | | Hz, 44H), 2.92(dd, J = 5.9, 11.8 Hz, 2H), 2.46-2.25(m, 1H), 1.99(dd, J = 12.3, 10.1 Hz, |
| | | 1H), 1.78(d, J = 22.7 Hz, 2H), 1.46-1.27(m, 2H), 1.20(t, J = 7.3 Hz, 3H), 1.03(t, J =7.3 |
| | | Hz, 1H). LCMS [M + H]⁺ = 901.7 |
| NT04 | UB-181240 | 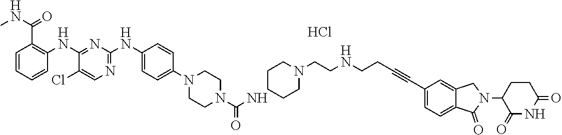 [SMILES: CNC(=O)c1ccccc1Nc1nc(Nc2ccc(N3CCN(C(=O)NC4CCN(CCNCCC#Cc5ccc6c(c5)CN(C5CCC(=O)NC5=O)C6=O)CC4)CC3)cc2)ncc1Cl ; Cl] |
| | | ¹H NMR(400 MHz, DMSO-d₆) δ 12.00(s, 1H), 11.02(s, 1H), 10.93(s, 1H), 10.17(s, 1H), |
| | | 10.01(s, 2H), 9.01(q, J = 4.4 Hz, 1H), 8.61(d, J = 8.3 Hz, 1H),8.32(s, 1H), 7.85(d, J = 7.9 |
| | | Hz, 1H), 7.77-7.59(m, 7H), 7.53(s, 1H), 7.19(s, 1H), 6.95(d, J = 20.9 Hz, 1H), 5.16-5.06 |
| | | (m, 1H), 4.51-4.33(m, 2H), 3.59(s, 2H), 3.54-3.48(m, 4H), 3.43(s, 4H), 3.25(s, 5H), 3.01 |
| | | (d, J = 7.2 Hz, 2H), 2.90(ddd, J = 17.9, 13.5, 5.2 Hz, 1H), 2.79(d, J = 4.3 Hz, 3H), 2.62- |
| | | 2.54(m, 1H), 2.38(qd, J = 13.2, 4.4 Hz, 1H), 2.03-1.81(m, 5H), 1.33-1.22(m, 4H). LCMS |
| | | [M + H]⁺ = 901.98 |
| NT05 | UB-181249 | 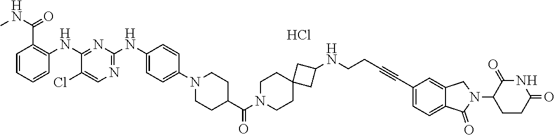 [SMILES: CNC(=O)c1ccccc1Nc1nc(Nc2ccc(N3CCC(C(=O)N4CCC5(CC4)CC(NCCC#Cc4ccc6c(c4)CN(C4CCC(=O)NC4=O)C6=O)C5)CC3)cc2)ncc1Cl ; Cl] |
| | | LC-MS [M/2 + H]⁺ = 443.9 |
| NT06 | UB-181250 | 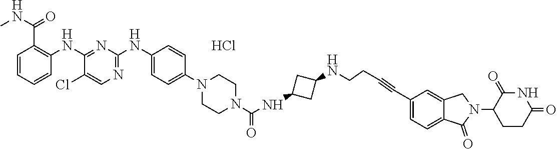 [SMILES: CNC(=O)c1ccccc1Nc1nc(Nc2ccc(N3CCN(C(=O)N[C@H]4C[C@@H](NCCC#Cc5ccc6c(c5)CN(C5CCC(=O)NC5=O)C6=O)C4)CC3)cc2)ncc1Cl ; Cl] |
| | | LC-MS [M/2 + H]⁺ = 423.2 |
| NT07 | UB-181251 | 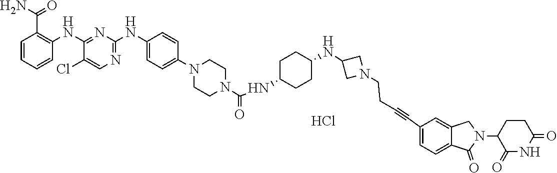 [SMILES: NC(=O)c1ccccc1Nc1nc(Nc2ccc(N3CCN(C(=O)N[C@H]4CC[C@@H](NC5CN(CCC#Cc6ccc7c(c6)CN(C6CCC(=O)NC6=O)C7=O)C5)CC4)CC3)cc2)ncc1Cl ; Cl] |
| | | LCMS [M + H]⁺ = 871.8 |
| NT08 | UB-181257 | 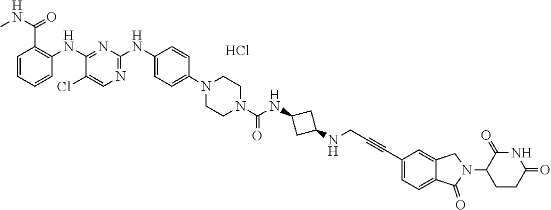 [SMILES: CNC(=O)c1ccccc1Nc1nc(Nc2ccc(N3CCN(C(=O)N[C@H]4C[C@@H](NCC#Cc5ccc6c(c5)CN(C5CCC(=O)NC5=O)C6=O)C4)CC3)cc2)ncc1Cl ; Cl] |
| | | ¹H NMR(400 MHz, DMSO-d₆) δ 11.76(s, 1H), 11.01(s, 1H), 9.66(s, 2H),9.49(s, 1H), |
| | | 8.82-8.68(m, 2H), 8.21(s, 1H), 7.83-7.73(m, 3H), 7.64(d, J = 7.9 Hz, 1H), 7.58-7.44(m, |
| | | 3H), 7.20-7.04(m, 3H), 6.99(m, 1H), 5.13(dd, J = 13.3, 5.1 Hz, 1H), 4.50(d, J = 17.7 Hz, |
| | | 1H), 4.37(d, J = 17.7 Hz, 1H), 4.17(t, J = 4.7 Hz, 2H), 3.93(dm, 1H), 3.54(m, 4H), 3.15 |
| | | (m,2H), 2.94-2.88(m, 1H), 2.81(d, J = 4.4 Hz, 3H), 2.59(m, 4H), 2.40(m, 2H), 2.23(m, |
| | | 2H), 2.06-1.96(m, 2H). LC-MS [M/2 + H]⁺ = 416 |
| NT09 | UB-181258 | 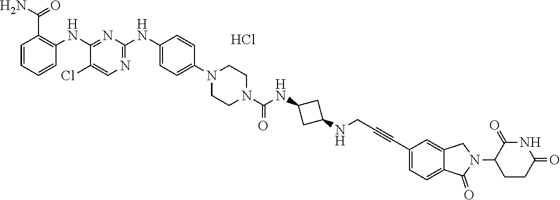 [SMILES: NC(=O)c1ccccc1Nc1nc(Nc2ccc(N3CCN(C(=O)N[C@H]4C[C@@H](NCC#Cc5ccc6c(c5)CN(C5CCC(=O)NC5=O)C6=O)C4)CC3)cc2)ncc1Cl ; Cl] |
| | | ¹H NMR(400 MHz, DMSO-d₆) δ 12.07(s, 1H), 11.01(s, 1H), 9.77(s, 2H), 9.62(s, 1H), |
| | | 8.74(s, 1H), 8.34(s, 1H), 8.23(s, 1H), 7.88-7.75(m, 4H), 7.66-7.46(m, 4H), 7.15(m, 3H), |
| | | 7.05(m, 1H), 5.13(dd, J = 13.3, 5.1 Hz, 1H), 4.50(d, J = 17.7 Hz, 1H), 4.37(d, J = 17.6 |
| | | Hz, 1H), 4.14(m, 2H), 3.94(m, 1H), 3.59(m, 4H), 3.21(m, 2H), 2.92(m, 2H), 2.60(m, |
| | | 4H), 2.40(m, 1H), 2.25(m, 2H), 2.02(m, 2H). LC-MS [M/2 + H]⁺ = 409 |
| NT10 | UB-181259 | 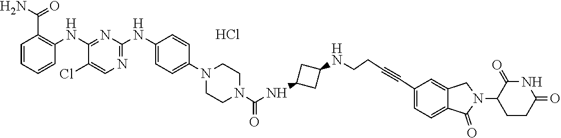 [SMILES: NC(=O)c1ccccc1Nc1nc(Nc2ccc(N3CCN(C(=O)N[C@H]4C[C@@H](NCCC#Cc5ccc6c(c5)CN(C5CCC(=O)NC5=O)C6=O)C4)CC3)cc2)ncc1Cl ; Cl] |
| | | ¹H NMR(400 MHz, DMSO-d₆) δ 12.22(s, 1H), 11.00(s, 1H), 9.94(s, 1H), 9.54(s, 2H), |
| | | 8.68(s, 1H), 8.38(s, 1H), 8.29(s, 1H), 7.92-7.80(m, 2H), 7.76-7.69(m, 2H), 7.61(m, |
| | | 3H), 7.52(t, J = 7.9 Hz, 1H), 7.43(s, 2H), 7.19(t, J = 7.5 Hz, 2H), 5.11(dd, J = 13.3, |
| | | 5.1 Hz, 1H), 4.47(d, J = 17.6 Hz, 1H), 4.34(d, J = 17.5 Hz, 1H), 3.93(m, 1H), 3.35 |
| | | (m, 4H), 3.09(t, J = 5.9 Hz, 2H), 2.97-2.91(m, 2H), 2.60(d, J = 11.9 Hz, 3H), 2.45- |
| | | 2.36(m, 2H), 2.28(m, 2H), 2.09-1.92(m, 2H). LC-MS [M/2 + H]⁺ = 416.6 |
| NT11 | UB-181261 | 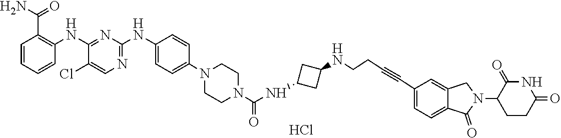 [SMILES: NC(=O)c1ccccc1Nc1nc(Nc2ccc(N3CCN(C(=O)N[C@H]4C[C@H](NCCC#Cc5ccc6c(c5)CN(C5CCC(=O)NC5=O)C6=O)C4)CC3)cc2)ncc1Cl ; Cl] |
| | | LCMS [M + H]⁺ = 830.1 |
| NT12 | UB-181269 | 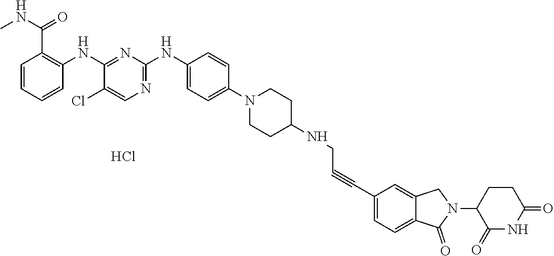 [SMILES: CNC(=O)c1ccccc1Nc1nc(Nc2ccc(N3CCC(NCC#Cc4ccc5c(c4)CN(C4CCC(=O)NC4=O)C5=O)CC3)cc2)ncc1Cl ; Cl] |
| | | ¹H NMR(400 MHz, DMSO-d₆) δ 11.80(s, 1H), 11.00(s, 1H), 9.78-9.69(m, 1H), 9.68- |
| | | 9.47(m,2H), 8.80(s, 1H), 8.46(s, 1H),8.16(s, 1H),7.87-7.66(m, 5H), 7.75(dt, J = 15.4, |
| | | 9.6 Hz, 1H), 7.63-7.49(m, 2H), 7.18(t, J = 7.5 Hz, 1H), 5.12(dd, J = 13.2, 5.0 Hz, |
| | | 1H), 4.56-4.35(m, 2H), 3.44-3.11(m, 4H), 3.00(s, 3H), 2.89-2.70(m,7H), 2.68(s, 3H), |
| | | 2.61 (d, J = 9.9 Hz, 2H), 2.35(dd, J = 3.3, 7.5 Hz, 4H), 2.19-2.08(m, 2H), 2.01-1.93 |
| | | (m, 2H). LCMS [M + H]⁺ = 746.8 |
| NT13 | UB-181270 | 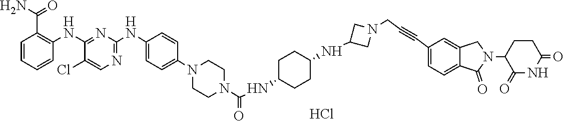 [SMILES: NC(=O)c1ccccc1Nc1nc(Nc2ccc(N3CCN(C(=O)N[C@H]4CC[C@@H](NC5CN(CC#Cc6ccc7c(c6)CN(C6CCC(=O)NC6=O)C7=O)C5)CC4)CC3)cc2)ncc1Cl ; Cl] |
| | | LCMS [M + H]⁺ = 900.2 |
| NT14 | UB-181272 | 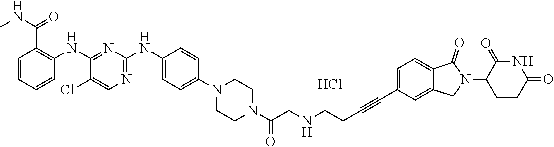 [SMILES: CNC(=O)c1ccccc1Nc1nc(Nc2ccc(N3CCN(C(=O)CNCCC#Cc4ccc5c(c4)CN(C4CCC(=O)NC4=O)C5=O)CC3)cc2)ncc1Cl ; Cl] |
| | | ¹H NMR(400 MHz, DMSO-d₆) δ 12.04(s, 1H), 11.02(s, 1H), 9.97(s, 1H), 9.34(s, |
| | | 2H), 8.91(d, J = 4.8 Hz, 1H), 8.64(s, 1H), 8.30(s, 1H), 7.82(dd, J = 8.0, 1.6 Hz, |
| | | 1H), 7.75-7.69(m, 2H), 7.62-7.46(m, 4H), 7.31-7.08(m, 3H), 5.12(dd, J = 13.3, 5.1 |
| | | Hz, 1H), 4.49-4.34(m, 2H), 4.24(d, J = 5.5 Hz,2H), 3.80(s, 2H), 3.68(s, 2H), 3.37 |
| | | (d, J = 18.0 Hz, 2H), 3.30-3.18(m, 4H), 3.00(t, J = 7.4 Hz, 2H), 2.93-2.87(m, 1H), |
| | | 2.81(d, J = 4.4 Hz, 3H), 2.66-2.56(m, 1H), 2.42-2.33(m, 1H), 2.05-1.96(m, 1H). |
| | | LCMS [M + H]⁺ = 789.9 |
| NT15 | UB-181273 | 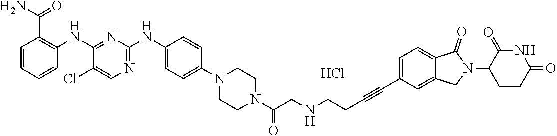 [SMILES: NC(=O)c1ccccc1Nc1nc(Nc2ccc(N3CCN(C(=O)CNCCC#Cc4ccc5c(c4)CN(C4CCC(=O)NC4=O)C5=O)CC3)cc2)ncc1Cl ; Cl] |
| | | ¹H NMR(400 MHz, DMSO-d₆) δ 12.23(s, 1H), 11.02(s, 1H), 9.86(s, 1H), 9.31(s, 2H), |
| | | 8.70(s, 1H), 8.40(s, 1H), 8.28(s, 1H), 7.90-7.79(m, 2H), 7.77-7.69(m, 2H), 7.63-7.45 |
| | | (m, 4H), 7.17(t, J = 7.6 Hz, 3H), 5.12(dd, J = 13.3, 5.1 Hz, 1H), 4.52-4.33(m, 2H), |
| | | 4.23(s, 2H), 3.77(s, 2H), 3.65(s, 2H), 3.31(s, 2H), 3.23(d, J = 7.9 Hz, 4H), 3.00(t, J = |
| | | 7.4 Hz, 2H), 2.94-2.86(m, 1H), 2.70-2.54(m, 1H), 2.40-2.33(m, 1H), 2.04-1.94(m, |
| | | 1H). LCMS [M + H]⁺ = 779.9 |
| NT16 | UB-181274 | 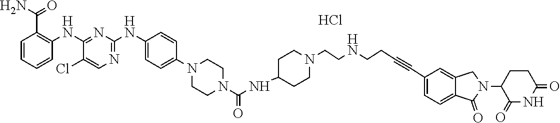 [SMILES: NC(=O)c1ccccc1Nc1nc(Nc2ccc(N3CCN(C(=O)NC4CCN(CCNCCC#Cc5ccc6c(c5)CN(C5CCC(=O)NC5=O)C6=O)CC4)CC3)cc2)ncc1Cl ; Cl] |
| | | ¹H NMR(400 MHz, DMSO-d₆) δ 12.23(s, 1H), 11.02(s, 1H), 9.85(s, 1H), 9.30(s, 2H), |
| | | 8.71(s, 1H), 8.39(s, 1H), 8.28(s, 1H), 7.88-7.80(m, 2H), 7.76-7.69(m, 2H), 7.62-7.45 |
| | | (m, 5H), 7.22-7.11(m, 3H), 5.12(dd, J = 13.3, 5.0 Hz, 1H), 4.50-4.30(m, 2H), 4.23(d, |
| | | J = 6.0 Hz, 2H), 3.30(s, 2H), 3.23(s,4H), 3.20-3.07(m, 2H), 3.03-2.81(m, 4H), 2.60(d, |
| | | J = 16.5 Hz, 1H), 2.41-2.33(m, 1H), 2.05-1.97(m, 1H). LCMS [M + H]⁺ = 887.6 |
| NT17 | UB-181279 | 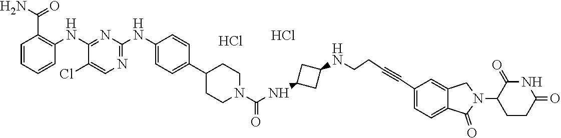 [SMILES: NC(=O)c1ccccc1Nc1nc(Nc2ccc(C3CCN(C(=O)N[C@H]4C[C@@H](NCCC#Cc5ccc6c(c5)CN(C5CCC(=O)NC5=O)C6=O)C4)CC3)cc2)ncc1Cl ; Cl ; Cl] |
| | | ¹H NMR(400 MHz, DMSO) δ 12.04(s, 1H), 11.00(s, 1H), 9.62(s, 1H), 9.40(s, 2H), |
| | | 8.74(d, J = 6.8 Hz, 1H), 8.35(d, J = 9.9 Hz, 1H), 8.23(t, J = 3.3 Hz, 1H), 7.84(d, J = |
| | | 7.9 Hz, 1H), 7.78(s, 1H), 7.75-7.69(m, 2H), 7.60(d, J = 8.0 Hz, 1H), 7.55(d, J = 8.3 |
| | | Hz, 2H), 7.48(t, J = 7.3 Hz, 1H), 7.16(t, J = 8.6 Hz, 3H), 6.86(s, 1H), 5.11(dd, J = |
| | | 13.3, 5.1 Hz, 1H), 4.40(dd, J = 51.1, 17.7 Hz, 2H), 4.13(d, J = 12.4 Hz, 2H), 3.89(d, |
| | | J = 7.9 Hz, 2H), 3.53-3.43(m, 1H), 3.09(d, J = 5.5 Hz, 2H), 2.97-2.83(m, 3H), 2.79- |
| | | 2.54(m, 6H), 2.45-2.31(m, 2H), 2.24(d, J = 9.8 Hz, 2H), 2.03-1.96(m, 1H), 1.74(d, |
| | | J = 12.5 Hz, 2H), 1.53-1.40(m, 2H). LC-MS [M/2 + H]⁺ = 415.7 |
| NT18 | UB-181315 | 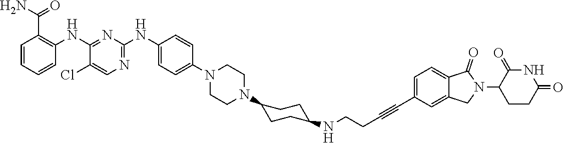 |
| | | LCMS [M + H]⁺ = 816.9 |
| NT19 | | 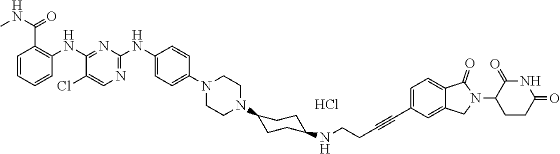 |
| NT20 | UB-181363 | 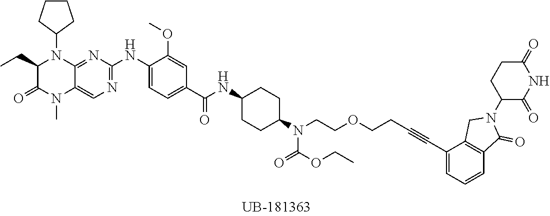 [SMILES: CCOC(=O)N(CCOCCC#Cc1cccc2c1CN(C1CCC(=O)NC1=O)C2=O)[C@H]1CC[C@@H](NC(=O)c2ccc(Nc3ncc4c(n3)N(C3CCCC3)[C@H](CC)C(=O)N4C)c(OC)c2)CC1] |
| NT21 | UB-181364 | 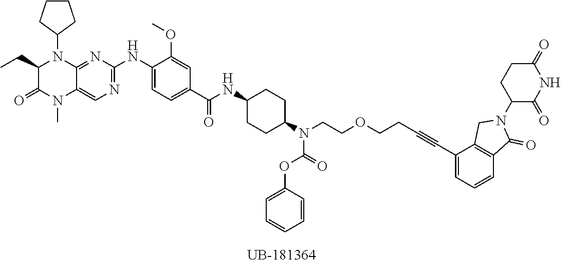 [SMILES: CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)C(=O)Oc5ccccc5)CC4)cc3OC)nc2N1C1CCCC1] |
| NT22 | UB-181332 | 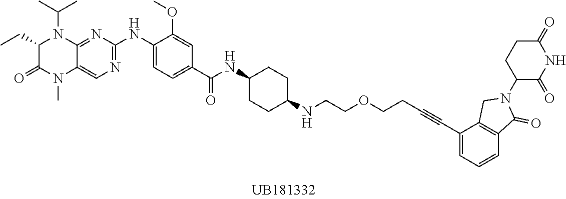 [SMILES: CC[C@H]1C(=O)N(C)c2cnc(Nc3ccc(C(=O)N[C@H]4CC[C@@H](NCCOCCC#Cc5cccc6c5CN(C5CCC(=O)NC5=O)C6=O)CC4)cc3OC)nc2N1C(C)C] |
| NT23 | UB-181333 | 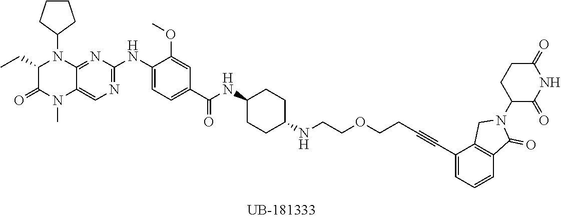 [SMILES: CC[C@H]1C(=O)N(C)c2cnc(Nc3ccc(C(=O)N[C@H]4CC[C@H](NCCOCCC#Cc5cccc6c5CN(C5CCC(=O)NC5=O)C6=O)CC4)cc3OC)nc2N1C1CCCC1] |
| NT24 | UB-181353 | 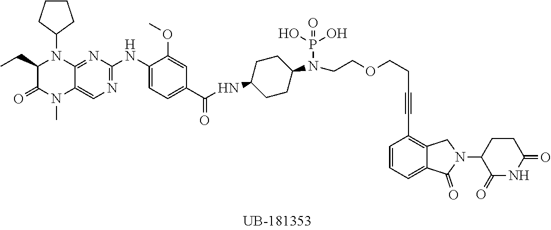 [SMILES: CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)P(=O)(O)O)CC4)cc3OC)nc2N1C1CCCC1] |
| NT25 | UB-181312 | 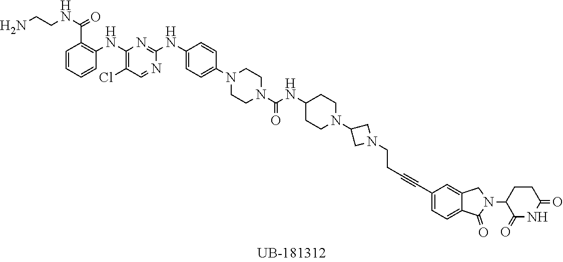 [SMILES: NCCNC(=O)c1ccccc1Nc1nc(Nc2ccc(N3CCN(C(=O)NC4CCN(C5CN(CCC#Cc6ccc7c(c6)CN(C6CCC(=O)NC6=O)C7=O)C5)CC4)CC3)cc2)ncc1Cl] |

TABLE E1

| | | |
|---|---|---|
| LT-1 | — | 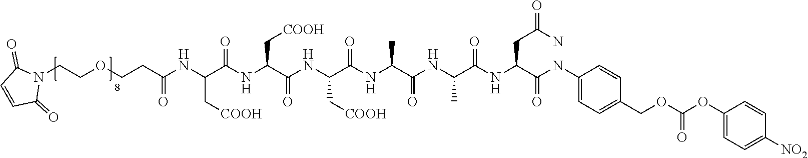 |
| LT-2 | — | 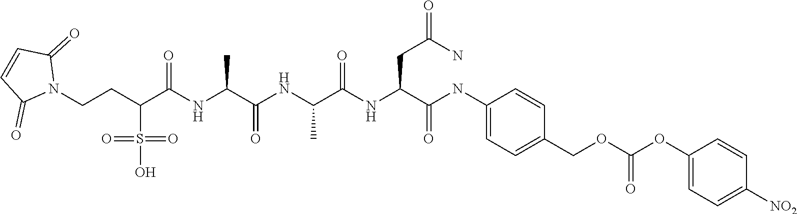 [SMILES: C[C@H](NC(=O)C(CCN1C(=O)C=CC1=O)S(=O)(=O)O)C(=O)N[C@@H](C)C(=O)N[C@@H](CC(N)=O)C(=O)Nc1ccc(COC(=O)Oc2ccc([N+](=O)[O-])cc2)cc1] |

TABLE E2

| | Compound | |
|---|---|---|
| No. | No. | Structure and Characterization Data |
| TED-L1 | UB-181237 | 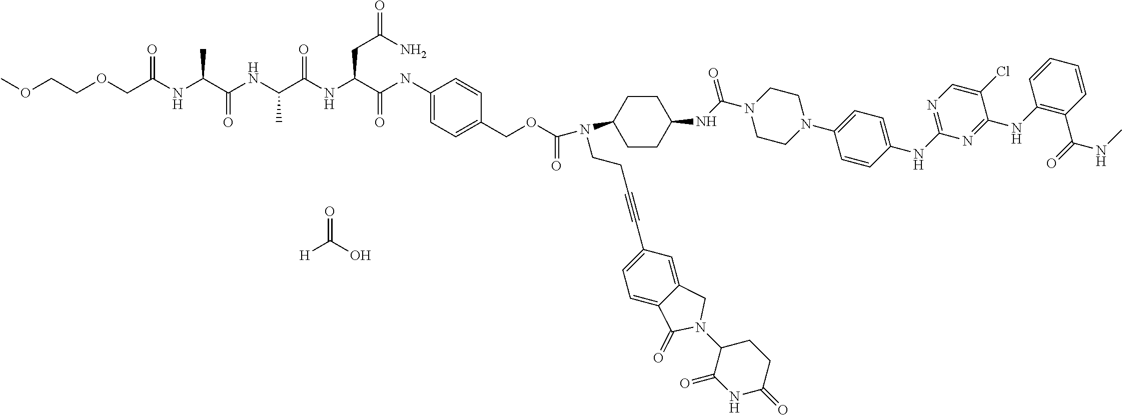 LCMS [M + H]+ = 1394.0. |
| TED-L2 | UB-181238 | 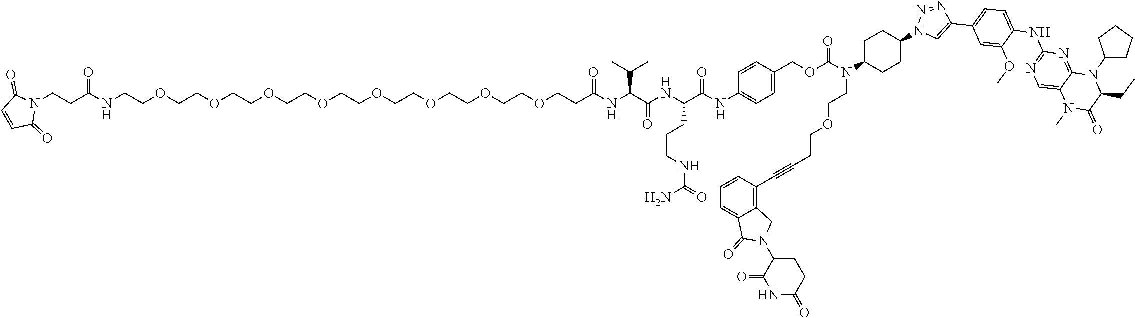 [SMILES: CC[C@H]1C(=O)N(C)c2cnc(Nc3ccc(-c4cn([C@H]5CC[C@@H](N(CCOCCC#Cc6cccc7c6CN(C6CCC(=O)NC6=O)C7=O)C(=O)OCc6ccc(NC(=O)[C@H](CCCNC(N)=O)NC(=O)[C@@H](NC(=O)CCOCCOCCOCCOCCOCCOCCOCCOCCNC(=O)CCN7C(=O)C=CC7=O)C(C)C)cc6)CC5)nn4)cc3OC)nc2N1C1CCCC1] LCMS [M + H]⁺ = 1866.0 |
| TED-L3 | UB-181242 | 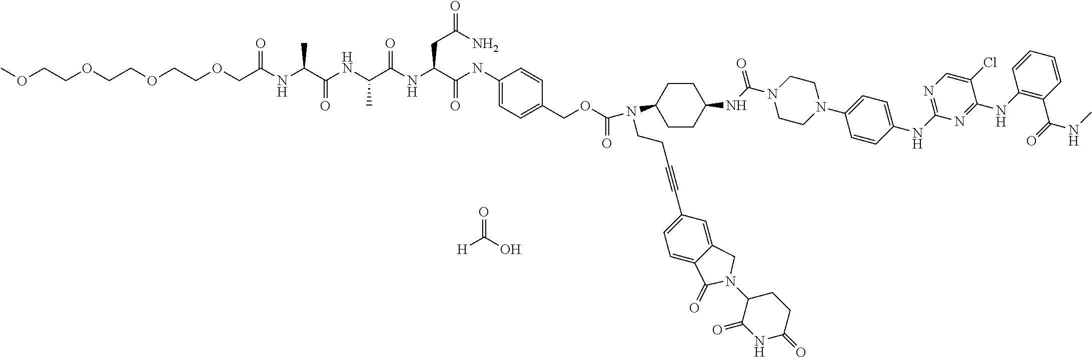 LCMS [M/2 + H]⁺ = 1482.1 |
| TED-L4 | UB-181295 | 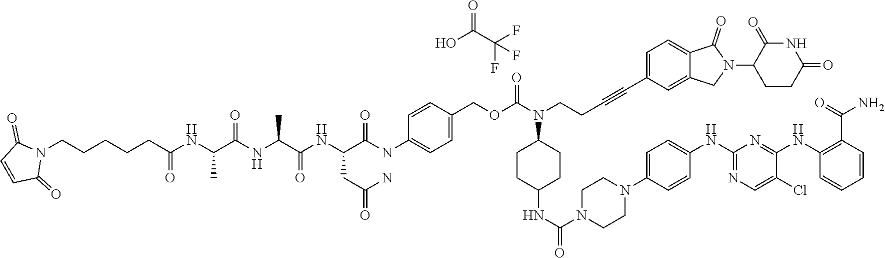 LCMS [M + H]+ = 1458.6 |
| TED-L5 | UB-181309 | 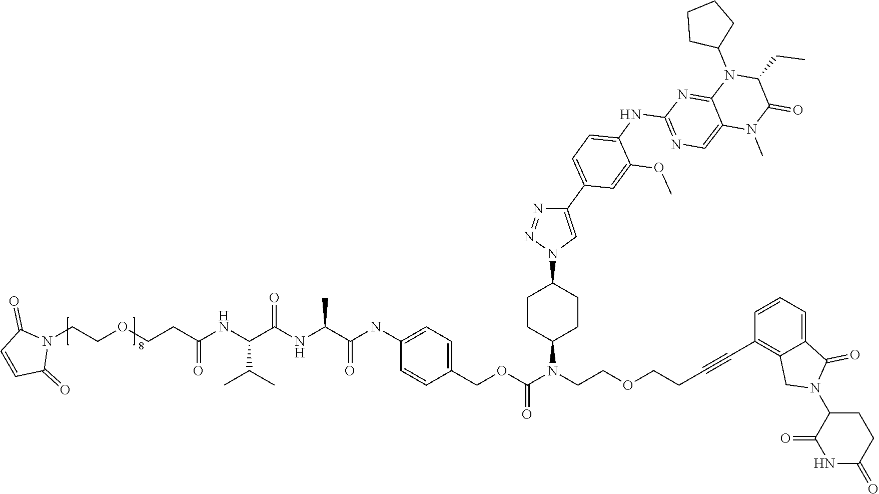 LCMS: [M + H] ⁺ = 1708. |
| TED-L6 | UB-181313 | 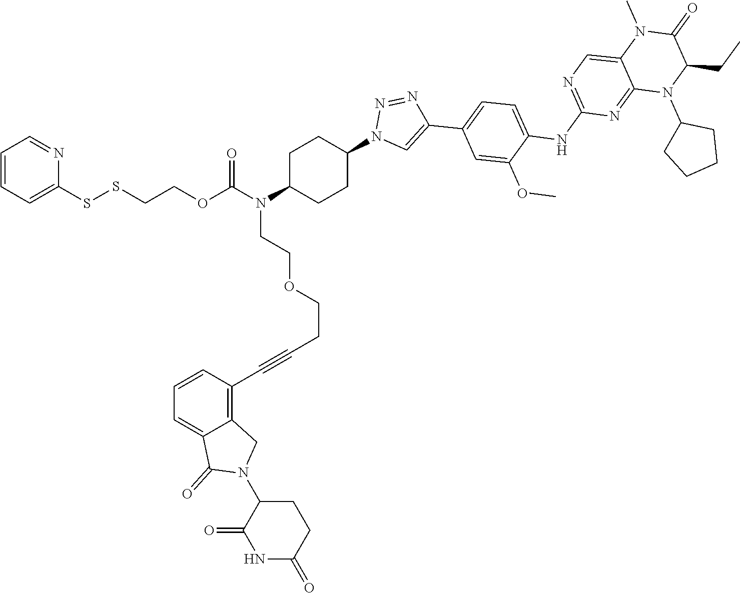 [SMILES: CC[C@@H]1C(=O)N(C)c2cnc(Nc3ccc(-c4cn([C@H]5CC[C@@H](N(CCOCCC#Cc6cccc7c6CN(C6CCC(=O)NC6=O)C7=O)C(=O)OCCSSc6ccccn6)CC5)nn4)cc3OC)nc2N1C1CCCC1] LCMS [M + H]⁺ = 1098.3 |
| TED-L7 | UB-181302 | 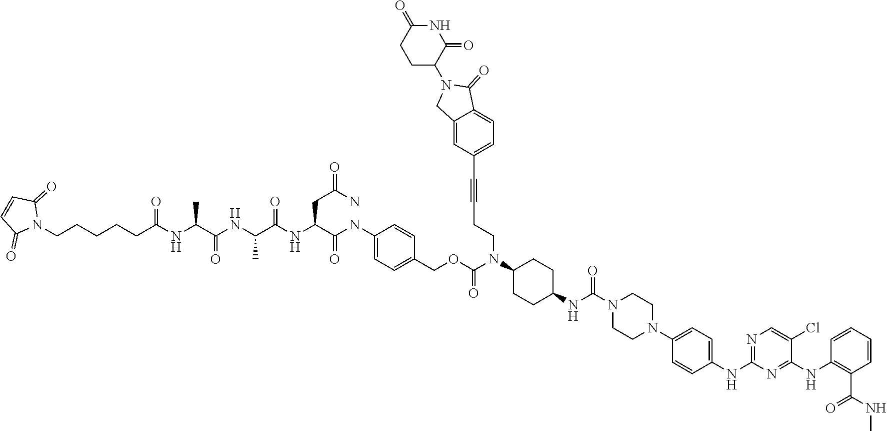 [SMILES: CNC(=O)c1ccccc1Nc1nc(Nc2ccc(N3CCN(C(=O)N[C@H]4CC[C@@H](N(CCC#Cc5ccc6c(c5)CN(C5CCC(=O)NC5=O)C6=O)C(=O)OCc5ccc(NC(=O)[C@H](CC(N)=O)NC(=O)[C@H](C)NC(=O)[C@H](C)NC(=O)CCCCCN6C(=O)C=CC6=O)cc5)CC4)CC3)cc2)ncc1Cl] UB-181302 |
| TED-L7 | UB-181325 Py-S-S-1189 | 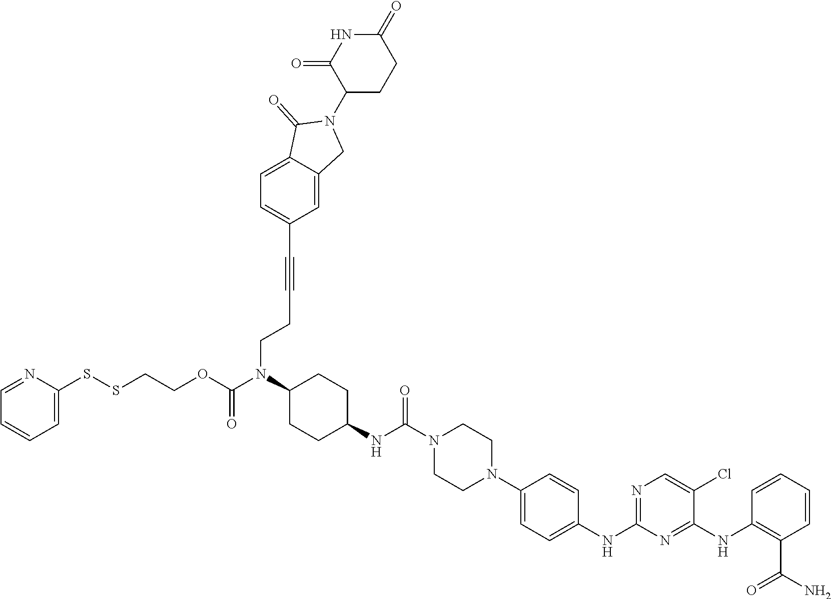 [SMILES: NC(=O)c1ccccc1Nc1nc(Nc2ccc(N3CCN(C(=O)N[C@H]4CC[C@@H](N(CCC#Cc5ccc6c(c5)CN(C5CCC(=O)NC5=O)C6=O)C(=O)OCCSSc5ccccn5)CC4)CC3)cc2)ncc1Cl] UB-181325 |
| TED-L8 | UB-181326 Py-S-S-1103 | 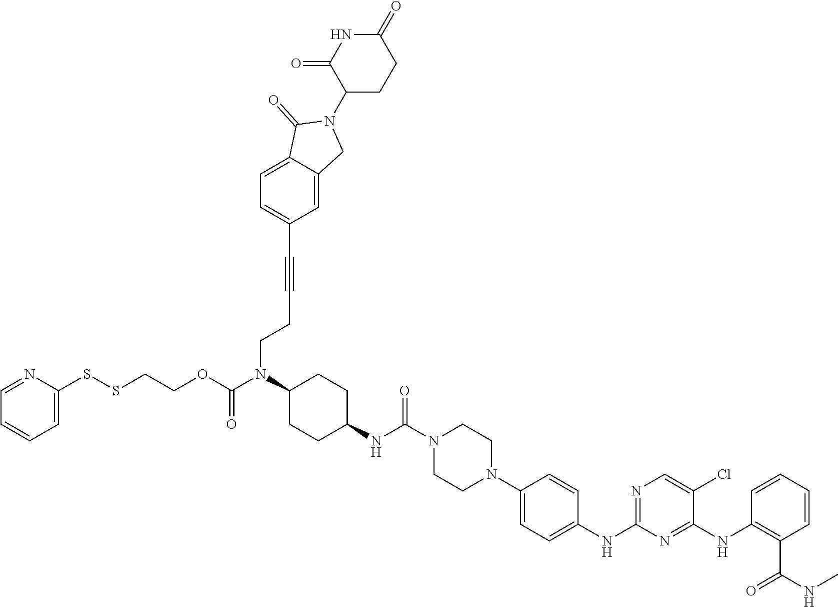 [SMILES: CNC(=O)c1ccccc1Nc1nc(Nc2ccc(N3CCN(C(=O)N[C@H]4CC[C@@H](N(CCC#Cc5ccc6c(c5)CN(C5CCC(=O)NC5=O)C6=O)C(=O)OCCSSc5ccccn5)CC4)CC3)cc2)ncc1Cl] UB-181326 |
| TED-L9 | UB-181355 | 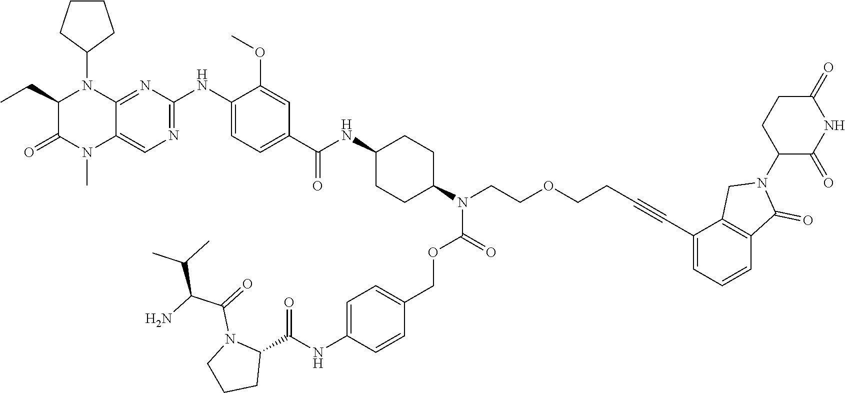 [SMILES: CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)C(=O)OCc5ccc(NC(=O)[C@@H]6CCCN6C(=O)[C@@H](N)C(C)C)cc5)CC4)cc3OC)nc2N1C1CCCC1] UB-181355 |
| TED-L10 | UB-181376 | 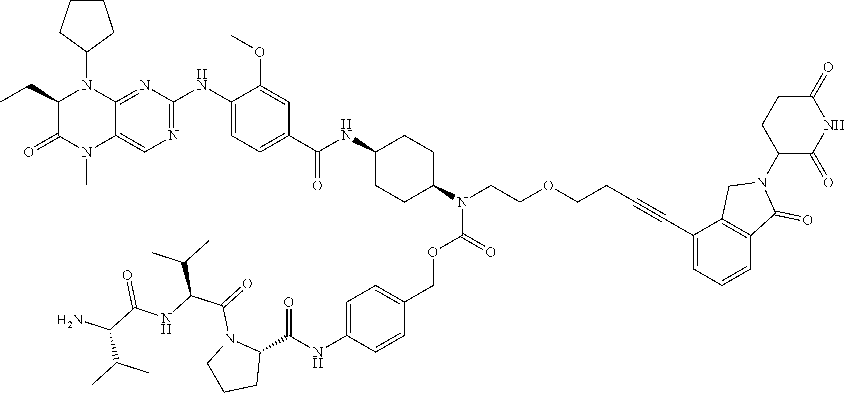 [SMILES: CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)C(=O)OCc5ccc(NC(=O)[C@@H]6CCCN6C(=O)[C@@H](NC(=O)[C@@H](N)C(C)C)C(C)C)cc5)CC4)cc3OC)nc2N1C1CCCC1] UB-181376 |
| TED-L11 | UB-181354 | 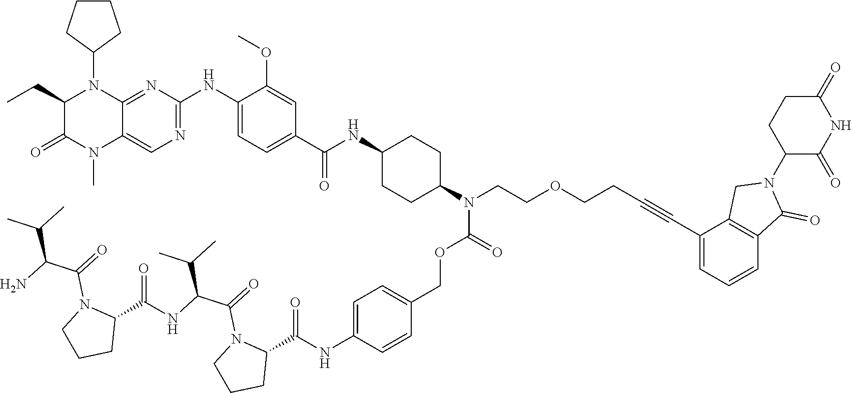 [SMILES: CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)C(=O)OCc5ccc(NC(=O)[C@@H]6CCCN6C(=O)[C@@H](NC(=O)[C@@H]6CCCN6C(=O)[C@@H](N)C(C)C)C(C)C)cc5)CC4)cc3OC)nc2N1C1CCCC1] UB-181354 |
| TED-L12 | UB-181362 | 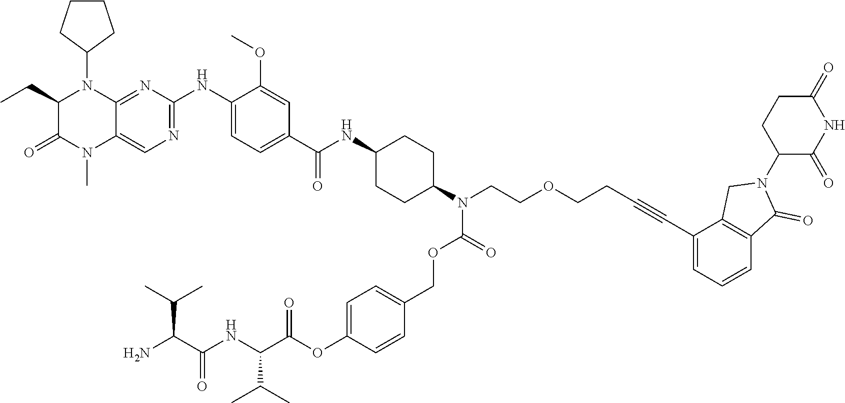 [SMILES: CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)C(=O)OCc5ccc(OC(=O)[C@@H](NC(=O)[C@@H](N)C(C)C)C(C)C)cc5)CC4)cc3OC)nc2N1C1CCCC1] UB-181362 |
| TED-L13 | UB-181313 | 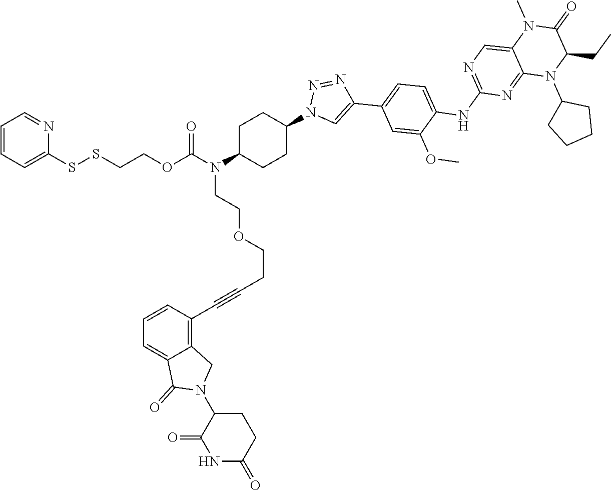 [SMILES: CC[C@@H]1C(=O)N(C)c2cnc(Nc3ccc(-c4cn([C@H]5CC[C@@H](N(CCOCCC#Cc6cccc7c6CN(C6CCC(=O)NC6=O)C7=O)C(=O)OCCSSc6ccccn6)CC5)nn4)cc3OC)nc2N1C1CCCC1] UB-181313 |
| TED-L14 | UB-181359 | 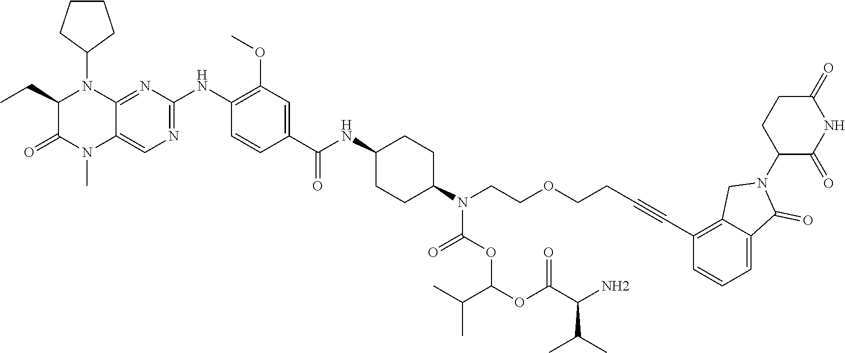 [SMILES: CC[C@@H]1C(=O)N(C)c2cnc(Nc3ccc(C(=O)N[C@H]4CC[C@@H](N(CCOCCC#Cc5cccc6c5CN(C5CCC(=O)NC5=O)C6=O)C(=O)OC(OC(=O)[C@@H](N)C(C)C)C(C)C)CC4)cc3OC)nc2N1C1CCCC1] UB-181359 |
| TED-L15 | UB-181375 | 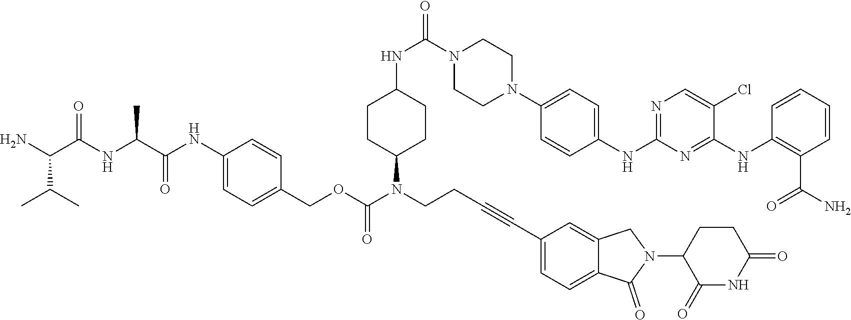 UB-181375 |
| TED-L16 | UB-181380 | 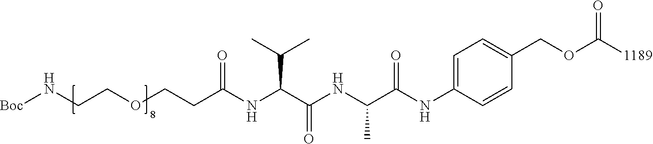 (Boc)NH-PEG8-VA-PAB-1189 UB-181380 |
| TED-L17 | UB-181381 | 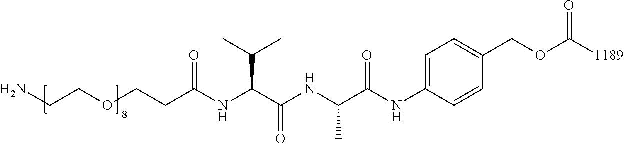 NH2-PEG8-VA-PAB-1189 UB-181381 |
| TED-L18 | UB-181391 | 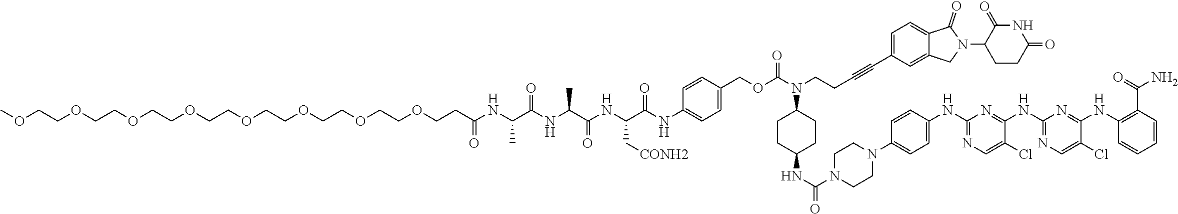 [SMILES: COCCOCCOCCOCCOCCOCCOCCOCCC(=O)N[C@@H](C)C(=O)N[C@@H](C)C(=O)N[C@@H](CC(N)=O)C(=O)Nc1ccc(COC(=O)N(CCC#Cc2ccc3c(c2)CN(C2CCC(=O)NC2=O)C3=O)[C@H]2CC[C@@H](NC(=O)N3CCN(c4ccc(Nc5ncc(Cl)c(Nc6ncc(Cl)c(Nc7ccccc7C(N)=O)n6)n5)cc4)CC3)CC2)cc1] UB-181391 |
| TED-L19 | UB-181387 | 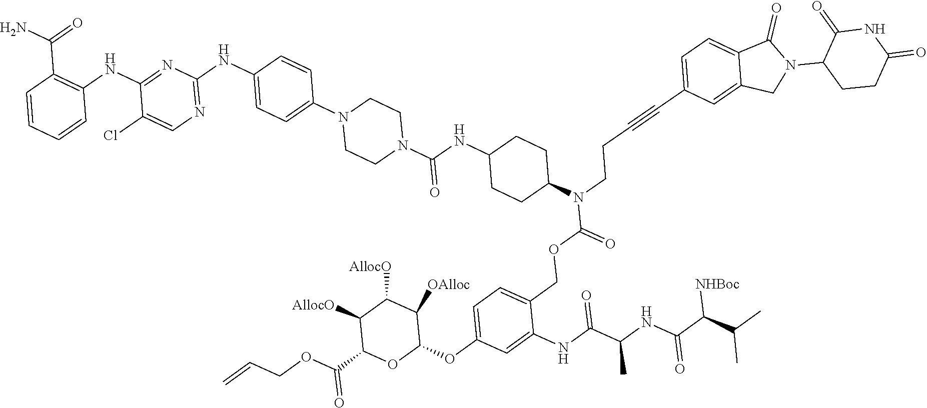 UB-181387 |
| TED-L20 | UB-181388 | 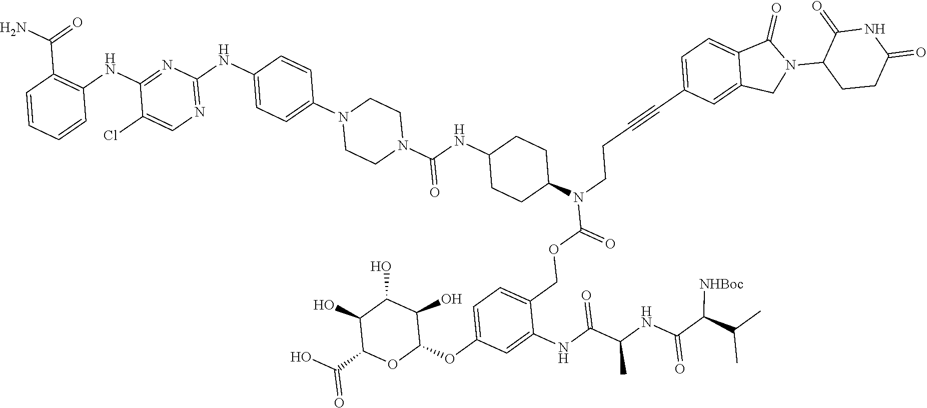 UB-181388 |
| TED-L21 | UB-181389 | 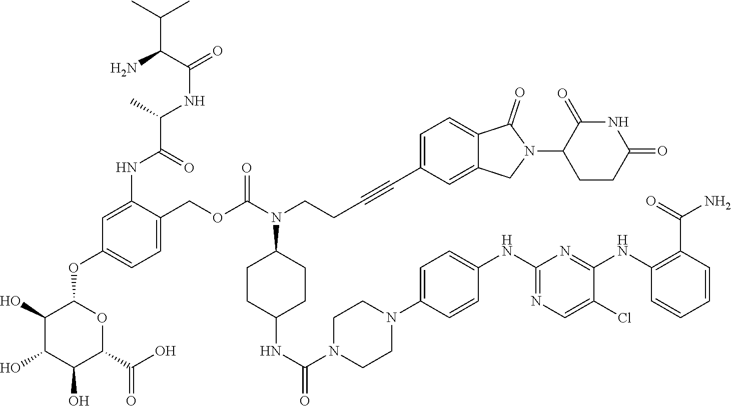 UB-181389 |
| TED-L22 | UB-181394 | 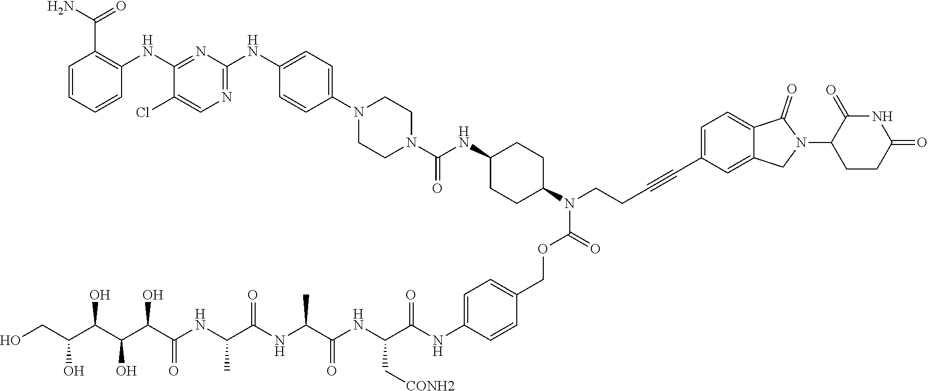 [SMILES: C[C@H](NC(=O)[C@H](C)NC(=O)[C@H](O)[C@@H](O)[C@H](O)[C@H](O)CO)C(=O)N[C@@H](CC(N)=O)C(=O)Nc1ccc(COC(=O)N(CCC#Cc2ccc3c(c2)CN(C2CCC(=O)NC2=O)C3=O)[C@H]2CC[C@@H](NC(=O)N3CCN(c4ccc(Nc5ncc(Cl)c(Nc6ccccc6C(N)=O)n5)cc4)CC3)CC2)cc1] UB-181394 |
| TED-L23 | UB-181396 | 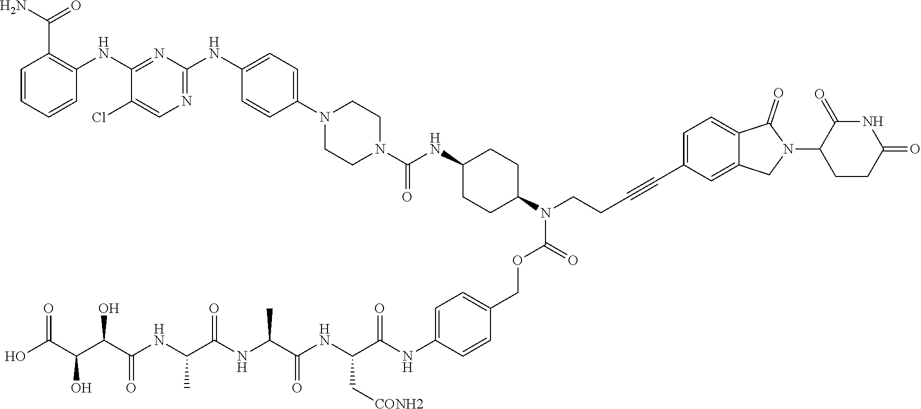 [SMILES: C[C@H](NC(=O)[C@H](C)NC(=O)[C@H](O)[C@@H](O)C(=O)O)C(=O)N[C@@H](CC(N)=O)C(=O)Nc1ccc(COC(=O)N(CCC#Cc2ccc3c(c2)CN(C2CCC(=O)NC2=O)C3=O)[C@H]2CC[C@@H](NC(=O)N3CCN(c4ccc(Nc5ncc(Cl)c(Nc6ccccc6C(N)=O)n5)cc4)CC3)CC2)cc1] UB-181396 |

TABLE D
Exemplary ACTED Conjugates

| | Com- | |
|---|---|---|
| | pound | |
| No. | No. | Structure and Characterization Data |
| OLW-1 | UB- 181243 | 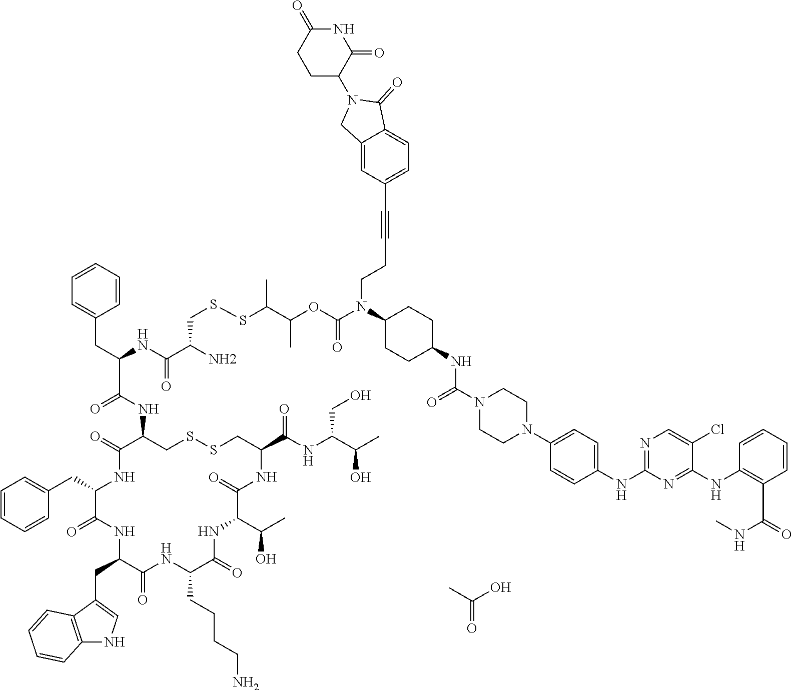 LCMS [M/2 + H]⁺ = 1064.2 |
| OLW-2 | UB- 181246 | 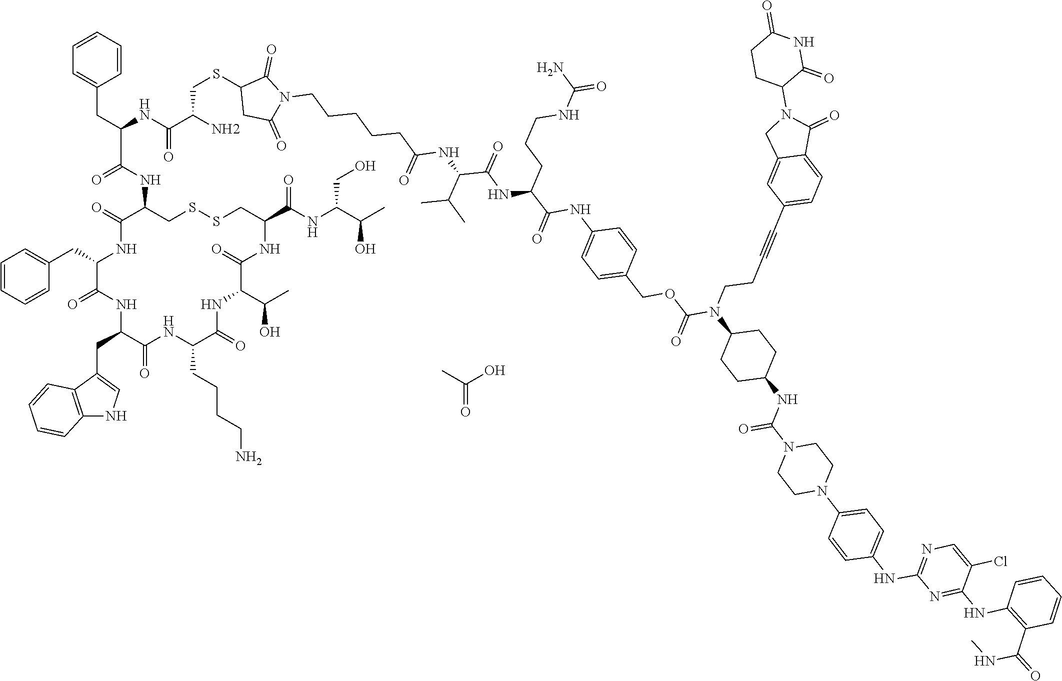 LCMS [M/2 + H]⁺ = 1298.4 |
| OLW-3 | UB- 181247 | 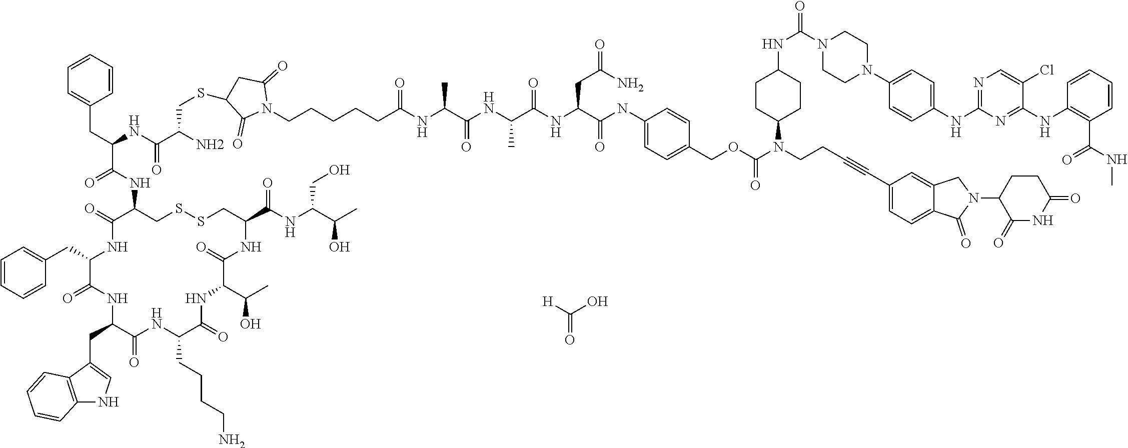 LCMS [M/2 + H]⁺ = 1297.6 |
| OLW-4 | UB- 181263 | 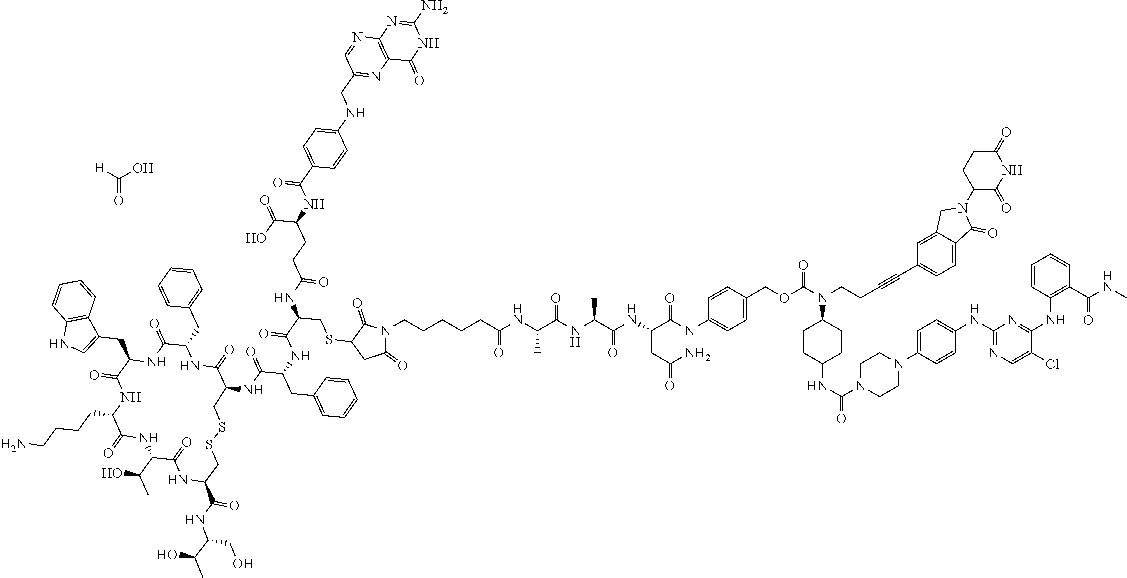 LCMS [M/2 + H]⁺ = 1510.2 |
| OLW-5 | UB- 181265 | 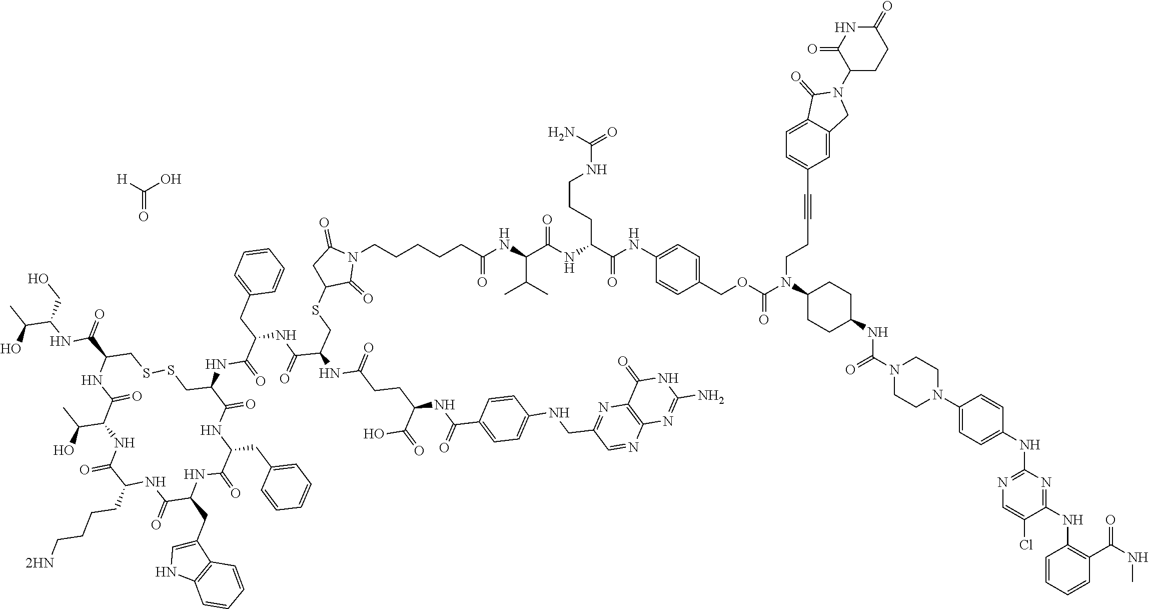 LCMS [M/2 + H]⁺ = 1511.4 |
| OLW-6 | UB- 181266 | 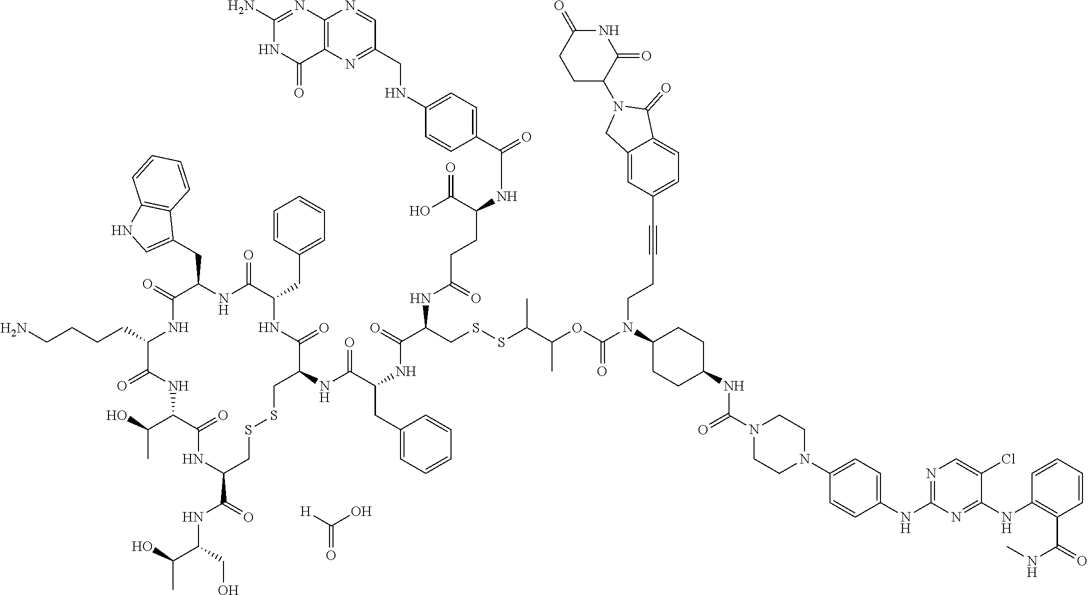 LCMS [M/2 + H]⁺ = 1275.6 |
| OLW-7 | UB- 181267 | 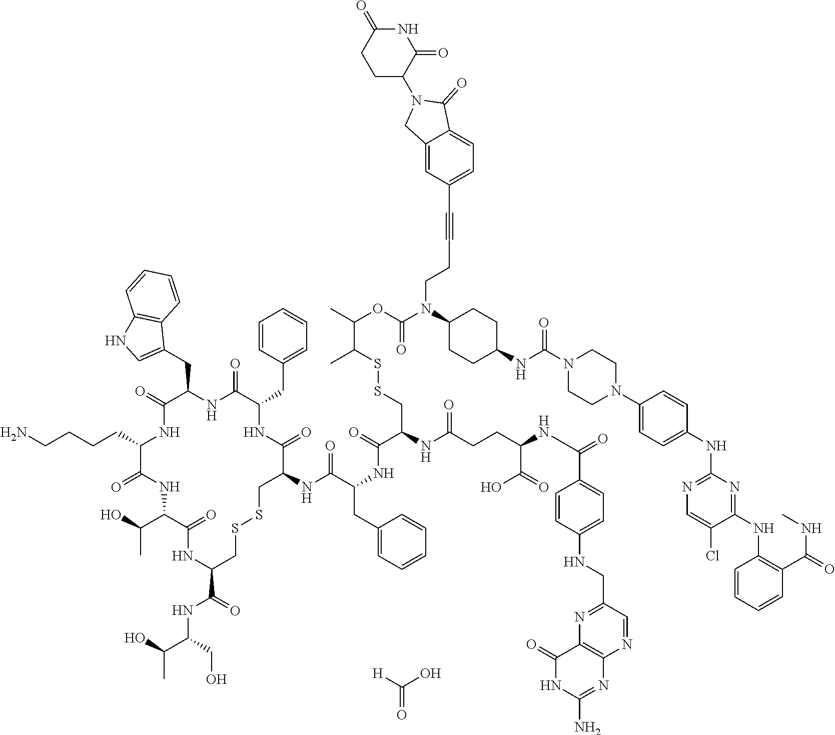 LCMS [M/2 + H]⁺ = 1275.6 |
| OLW-8 | UB- 181268 | 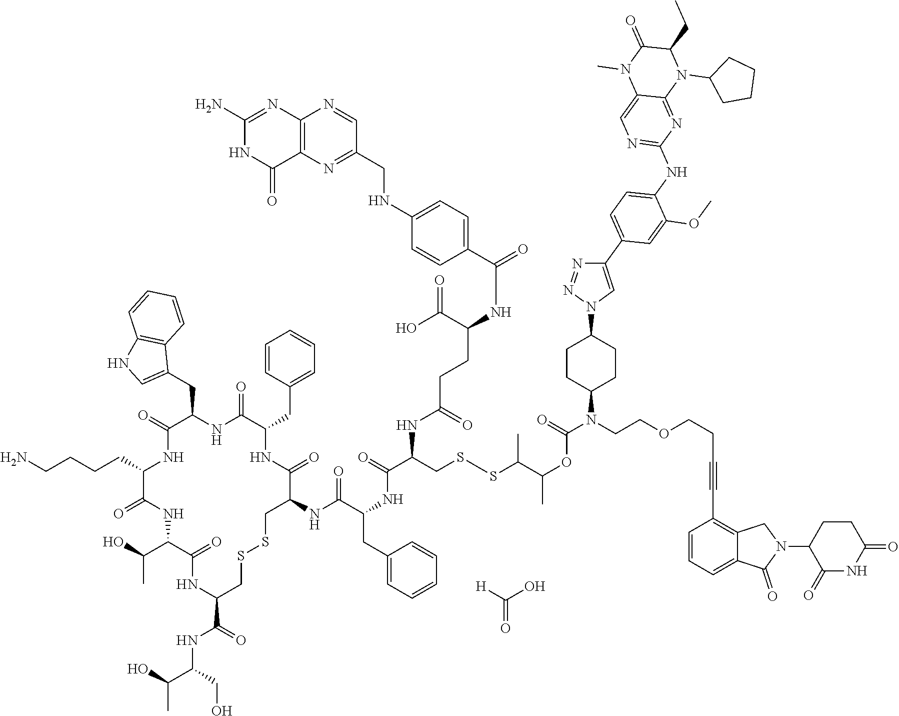 LCMS [M/2 + H]⁺ = 1281.2 |
| OLW-9 | UB- 181275 | 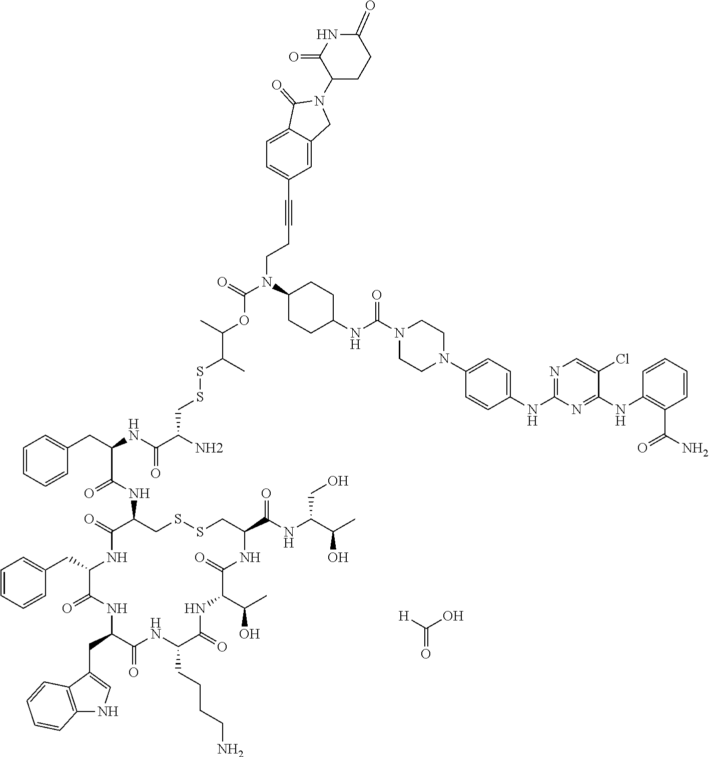 LCMS [M/2 + H]⁺ = 1056.8 |
| OLW-10 | UB- 181280 | 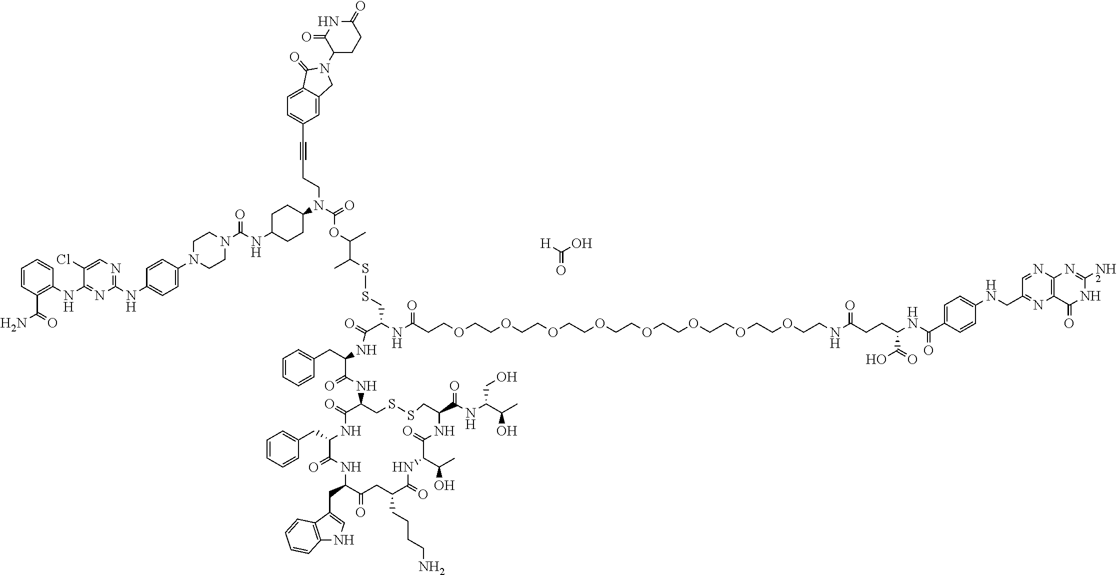 LCMS [M/3 + H]⁺ = 987.6 |
| OLW-11 | UB- 181285 | 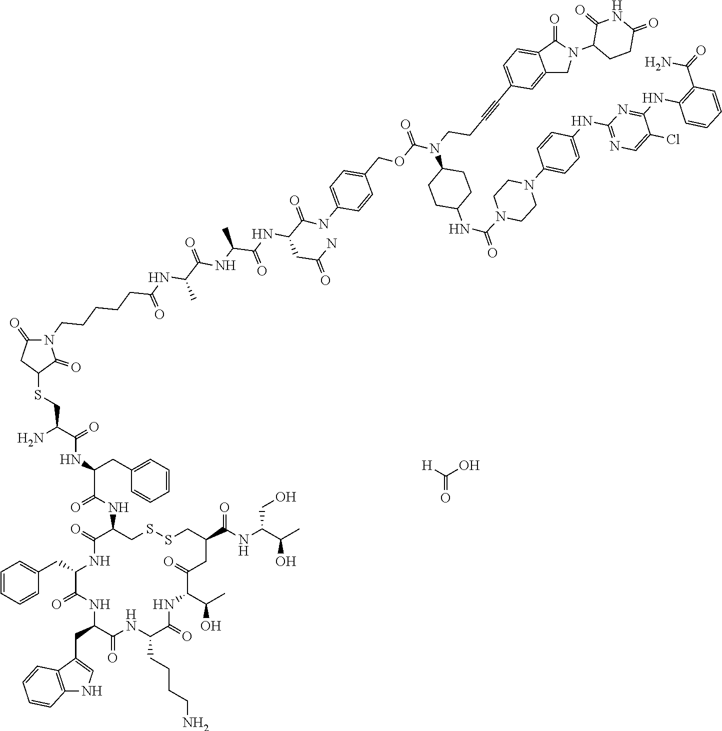 LCMS [M/3 + H]⁺ = 861.4 |
| OLW-12 | UB- 181289 | 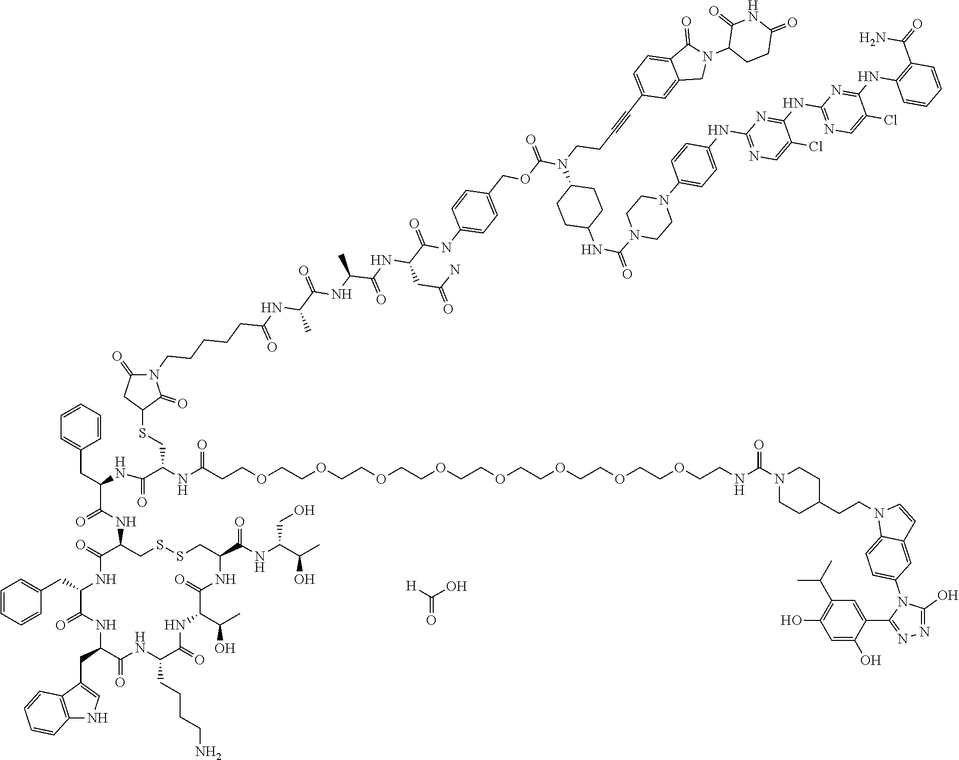 LCMS [M/3 + H]⁺ = 1165.2 |
| OLW-13 | UB- 181290 | 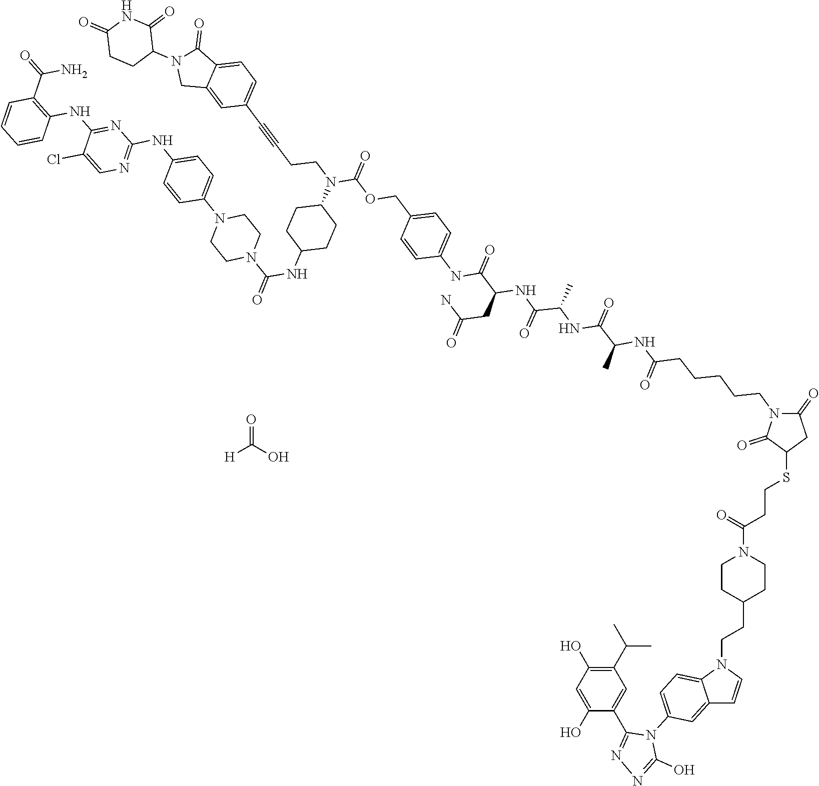 |
| | | ¹H NMR(400 MHZ, DMSO)δ 11.86(d, J = 15.4 Hz, 2H), 10.98(s, |
| | | 1H), 9.70(s, 1H), 9.55(d, J = 23.7 Hz, 2H), 9.22(s, 1H), 8.79(s, 1H), |
| | | 8.28(s, 2H), 8.16(d, J = 6.2 Hz, 2H), 8.11(d, J = 7.6 Hz, 1H), 8.01(d, |
| | | J = 7.0 Hz, 1H), 7.79(s, 1H), 7.73(s, 1H), 7.67(m, J = 16.0, 8.2 Hz, |
| | | 3H), 7.60(s, 1H), 7.52-7.43(m, 6H), 7.42(d, J = 1.8 Hz, 1H), 7.40(s, |
| | | 1H), 7.31(d, J = 8.2 Hz, 2H), 7.08(s, 1H), 6.98-6.87(m, 4H), 6.67(s, |
| | | 1H), 6.42(d, J = 2.9 Hz, 1H), 6.24(s, 1H), 6.12(d, J = 5.5 Hz, 1H), |
| | | 5.08(m, J = 15.0, 6.5 Hz, 3H), 4.61(d, J = 7.2 Hz, 1H), 4.41(d, J = |
| | | 17.7 Hz, 1H), 4.28(m, J = 16.4, 9.7 Hz, 3H), 4.20(m, J = 12.5, 6.7 |
| | | Hz, 3H), 4.00(d, J = 6.9 Hz, 1H), 3.78(s, 3H), 3.48(s, 5H), 3.17(m, |
| | | J = 18.4, 9.0 Hz, 2H), 3.03(s, 4H), 2.97-2.78(m, 6H), 2.76-2.56(m, |
| | | 10H), 2.45-2.31(m, 2H), 2.09(t, J = 7.2 Hz, 2H), 2.04-1.95(m, |
| | | 1H), 1.82(d, J = 10.6 Hz, 4H), 1.69(m, J = 18.7, 10.9 Hz, 4H), |
| | | 1.47(s, 9H), 1.21(t, J = 7.4 Hz, 9H), 0.81(t, J = 15.8 Hz, 6H). |
| | | LC-MS[M/2 + H] = 1005.17 |
| OLW-14 | UB- 181291 | 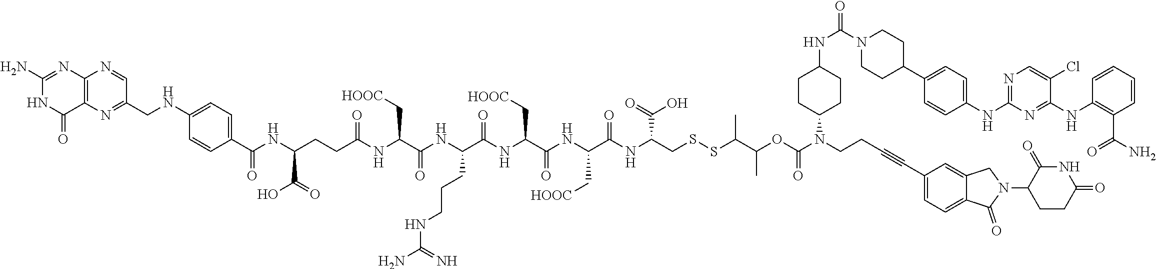 LCMS [M/2 + H]+ = 1018.9. |
| OLW-15 | UB- 181294 | 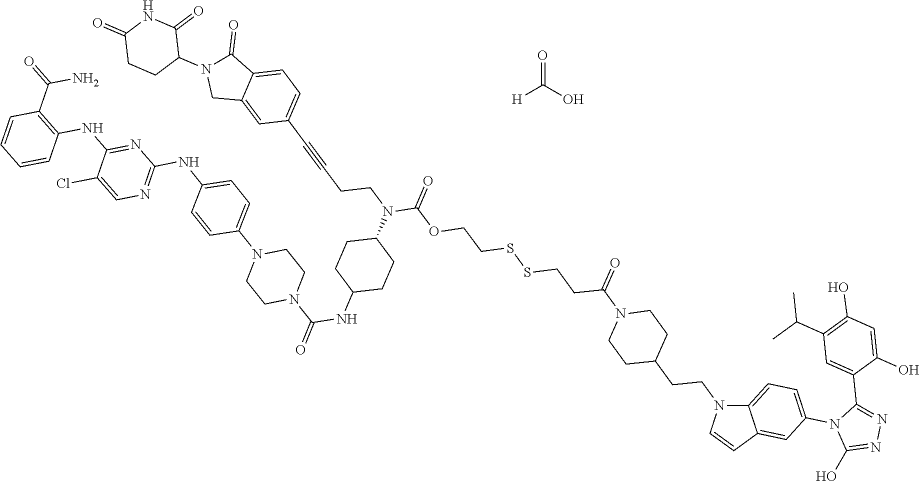 LC-MS[M/2 +H] = 756.90 |
| OLW-16 | UB- 181297 | 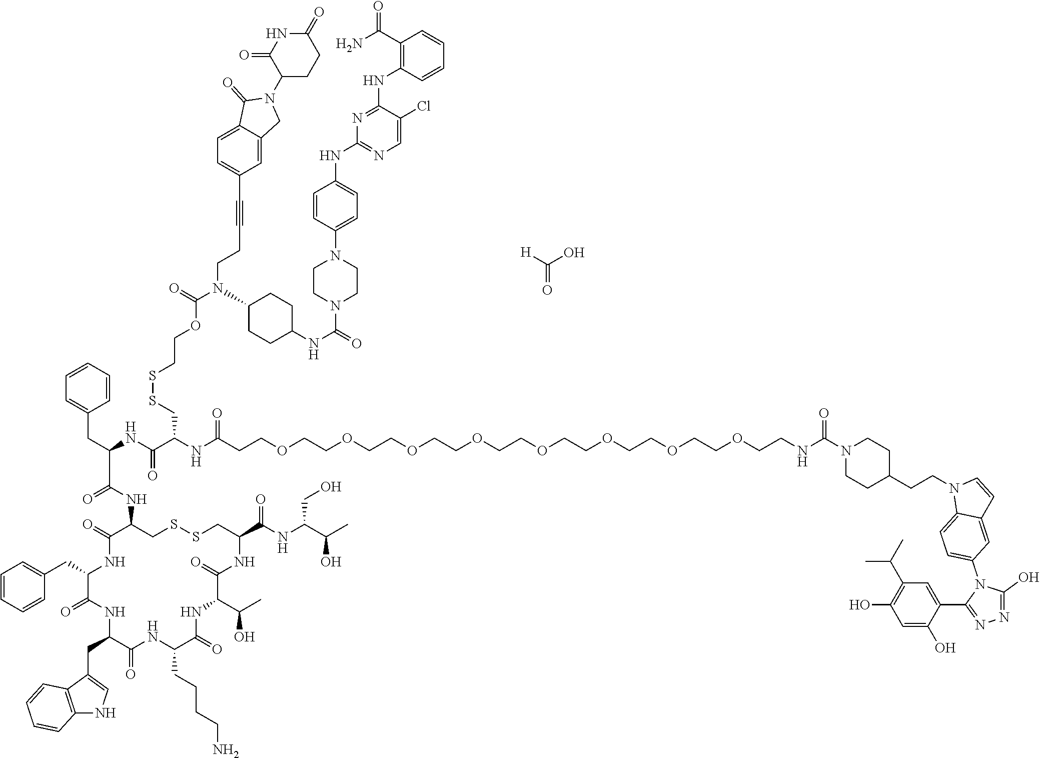 LCMS [M/2 + H]⁺ = 1499.2 |
| OLW-17 | UB- 181298 | 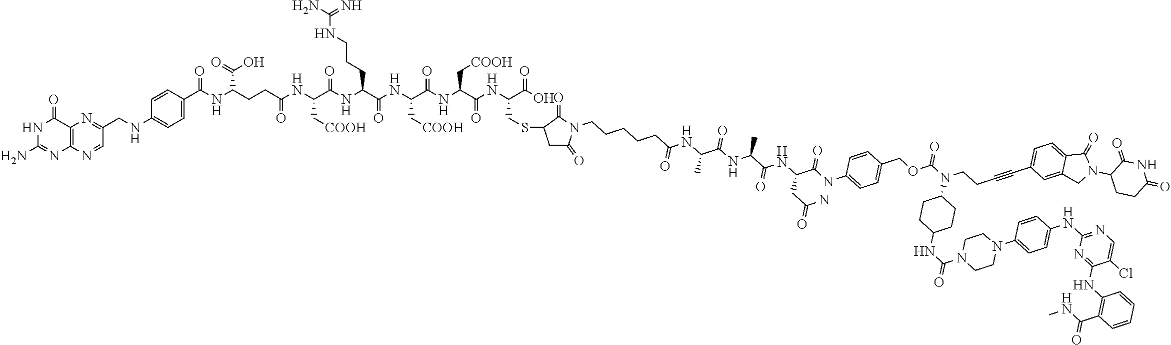 LCMS [M/2 + H]⁺ = 1260 |
| OLW-18 | UB- 181299 | 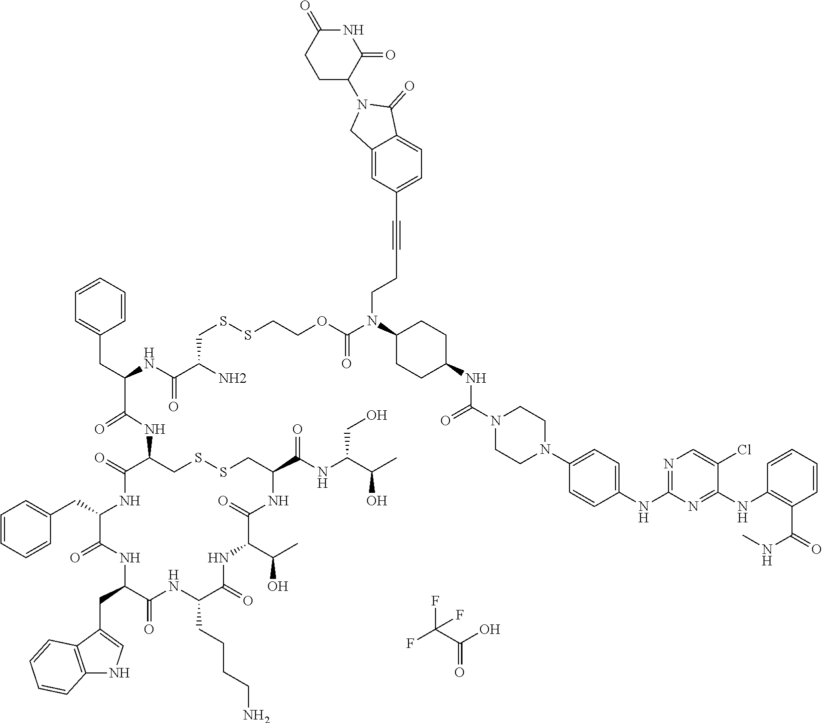 [SMILES: CNC(=O)c1ccccc1Nc1nc(Nc2ccc(N3CCN(C(=O)N[C@H]4CC[C@@H](N(CCC#Cc5ccc6c(c5)CN(C5CCC(=O)NC5=O)C6=O)C(=O)OCCSSC[C@H](N)C(=O)N[C@H](Cc5ccccc5)C(=O)N[C@H]5CSSC[C@@H](C(=O)N[C@H](CO)[C@@H](C)O)NC(=O)[C@H]([C@@H](C)O)NC(=O)[C@H](CCCCN)NC(=O)[C@@H](Cc6c[nH]c7ccccc67)NC(=O)[C@H](Cc6ccccc6)NC5=O)CC4)CC3)cc2)ncc1Cl ; O=C(O)C(F)(F)F] LCMS [M/2 + H]⁺ = 1050.2 |
| OLW-19 | UB- 181301 | 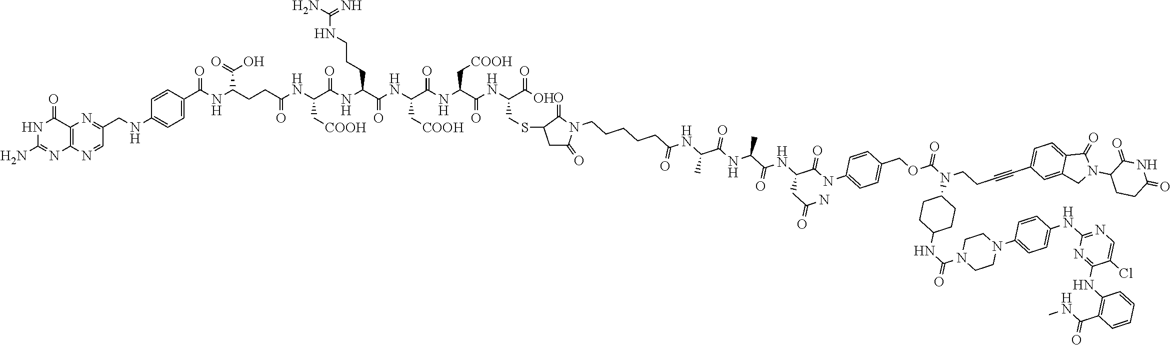 |
| | | 1H NMR(400 MHZ, DMSO)δ 11.84(s, 1H), 9.72(d, J = 22.3 Hz, 1H), |
| | | 9.22(s, 1H), 8.79(s, 1H), 8.63(s, 1H), 8.49(s, 2H), 8.28(s, 3H), |
| | | 8.15(s, 3H), 7.87(s, 1H), 7.80(d, J = 8.0 Hz, 2H), 7.75-7.55(m, |
| | | 10H), 7.52-7.38(m, 8H), 7.31(d, J = 8.3 Hz, 3H), 7.08(t, J = 7.8 Hz, |
| | | 2H), 6.90(d, J = 9.1 Hz, 5H), 6.62(d, J = 8.3 Hz, 2H), 6.11(s, 1H), |
| | | 5.08(dd, J = 14.7, 6.4 Hz, 3H), 4.61(d, J = 7.2 Hz, 2H), 4.54- |
| | | 4.35(m, 6H), 4.33-4.11(m, 7H), 4.00(d, J = 41.0 Hz, 3H), 3.78(s, |
| | | 3H), 3.47(s, 8H), 3.03(s, 6H), 2.68(dd, J = 7.7, 5.8 Hz, 3H), 2.57(d, |
| | | J = 6.6 Hz, 5H), 2.12(d, J = 24.8 Hz, 6H), 1.97(s, 4H), 1.83(s, 6H), |
| | | 1.64(s, 2H), 1.47(s, 12H), 1.21(t, J = 6.7 Hz, 9H). |
| | | LC-MS[M/2 + H] = 1253.05 |
| OLW-20 | UB- 181303 | 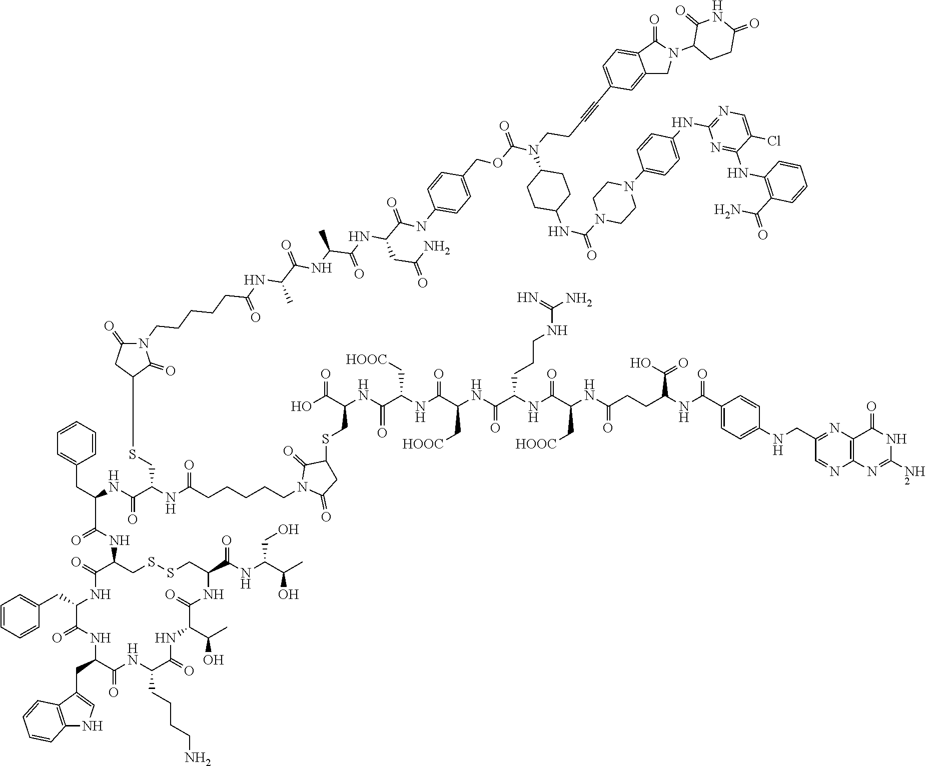 LCMS [M/3 + H] = 1275 |
| OLW-21 | UB- 181308 | 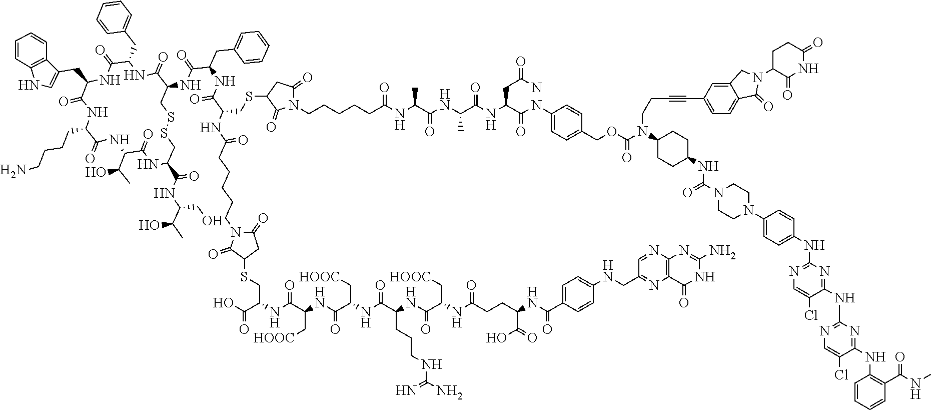 LC-MS[M/3 + H] = 1279.76 |
| OLW-22 | UB- 181310 | 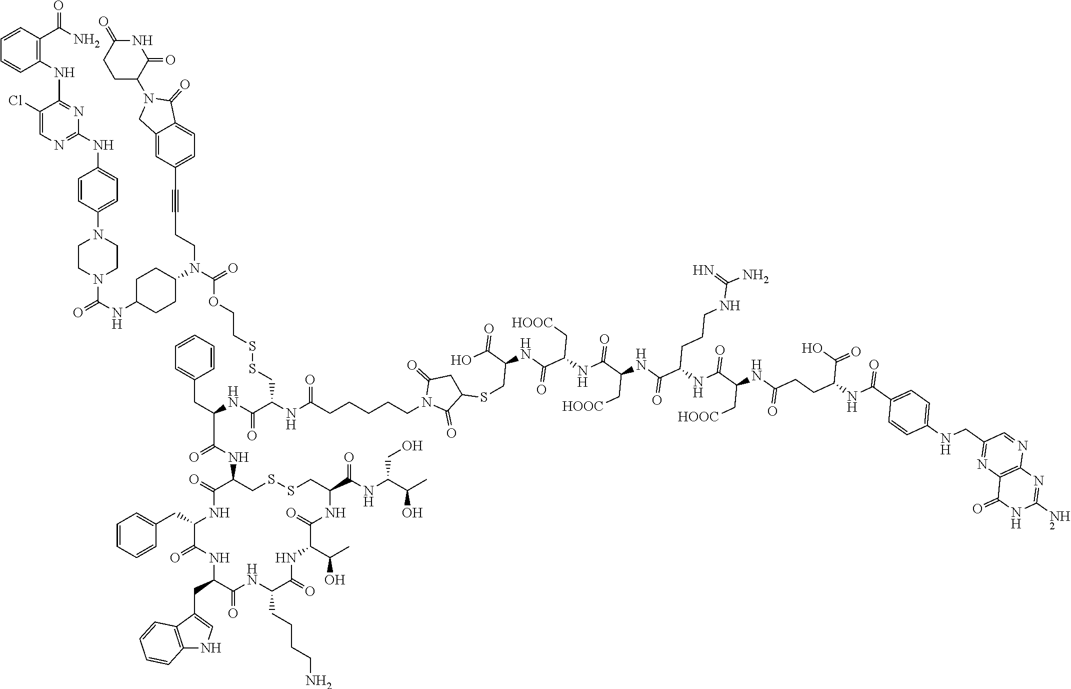 LC-MS[M/3 + H] = 1109.06 |
| OLW-23 | UB- 181311 | 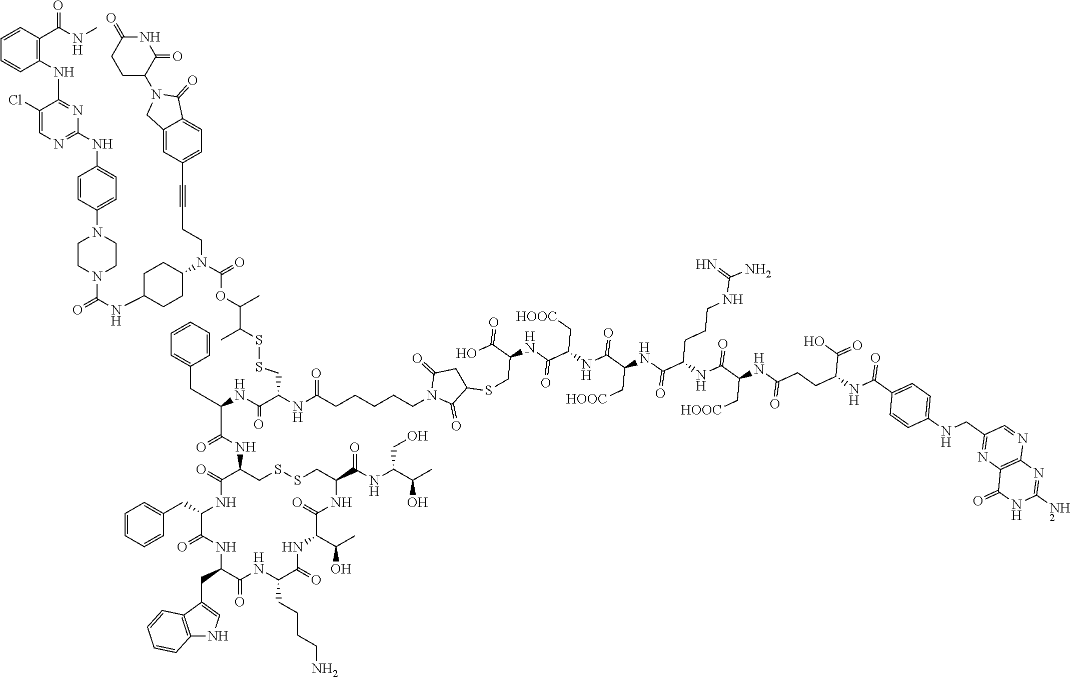 LC-MS[M/3 + H] = 1122.91 |
| OLW-24 | UB- 181321 | 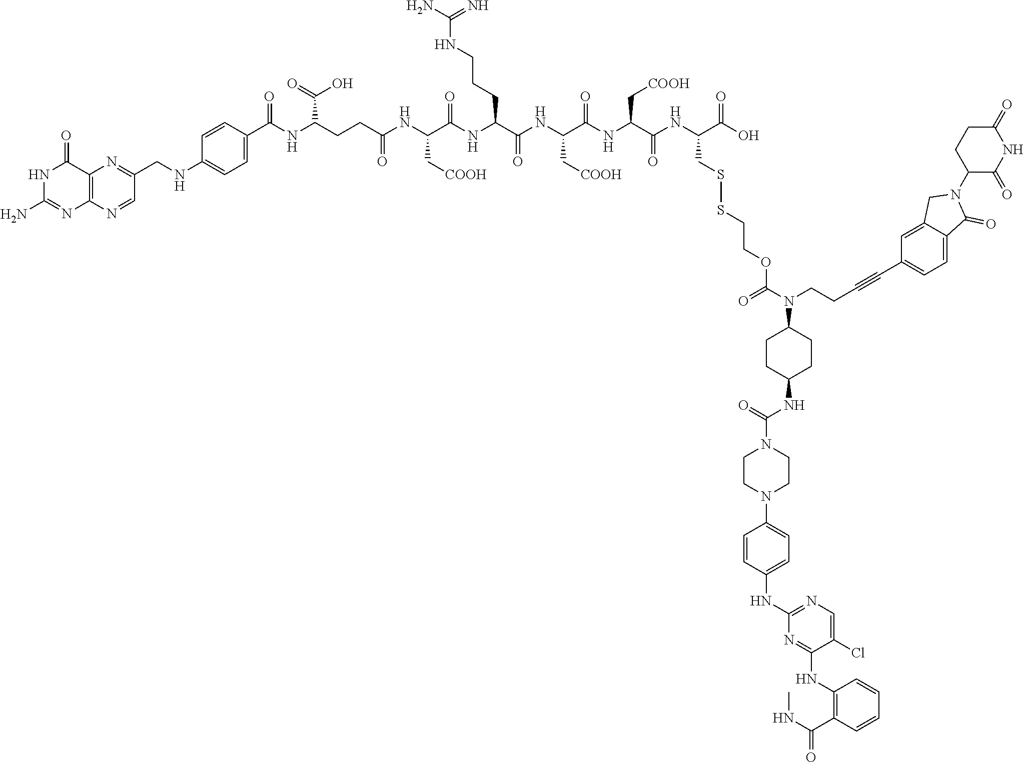 LCMS[M/2 + H] = 1011.33 |
| OLW-25 | UB- 181322 | 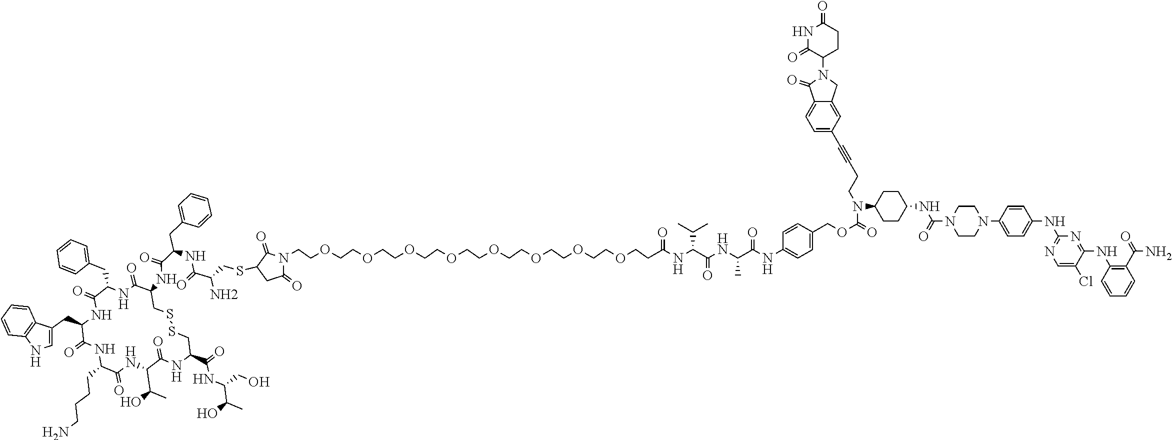 LCMS: [1/3M + 1] + = 935.9. |
| OLW-27 | UB- 181334 | 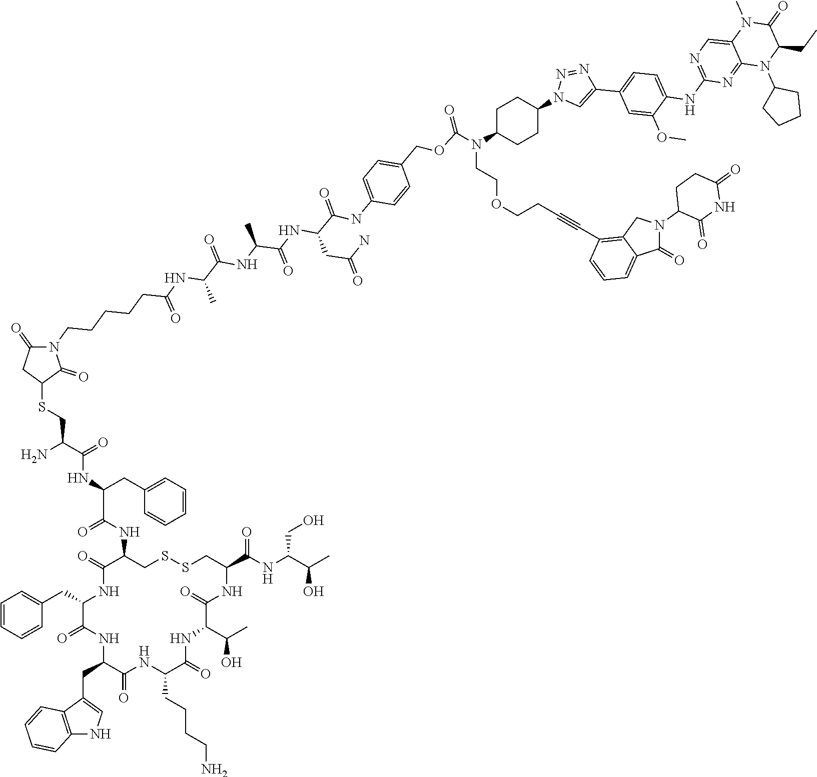 [SMILES: CC[C@@H]1C(=O)N(C)c2cnc(Nc3ccc(-c4cn([C@H]5CC[C@@H](N(CCOCCC#Cc6cccc7c6CN(C6CCC(=O)NC6=O)C7=O)C(=O)OCc6ccc(NC(=O)[C@H](CC(N)=O)NC(=O)[C@H](C)NC(=O)[C@H](C)NC(=O)CCCCCN7C(=O)CC(SC[C@H](N)C(=O)N[C@@H](Cc8ccccc8)C(=O)N[C@H]8CSSC[C@@H](C(=O)N[C@H](CO)[C@@H](C)O)NC(=O)[C@H]([C@@H](C)O)NC(=O)[C@H](CCCCN)NC(=O)[C@@H](Cc9c[nH]c%10ccccc9%10)NC(=O)[C@H](Cc9ccccc9)NC8=O)C7=O)cc6)CC5)nn4)cc3OC)nc2N1C1CCCC1] |
| OLW-28 | UB- 181335 | 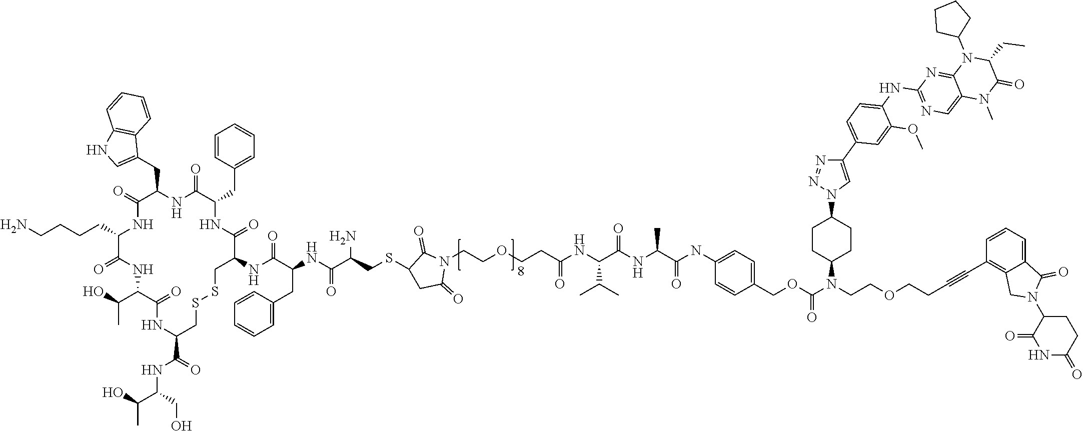 |
| OLW-29 | UB- 181336 | 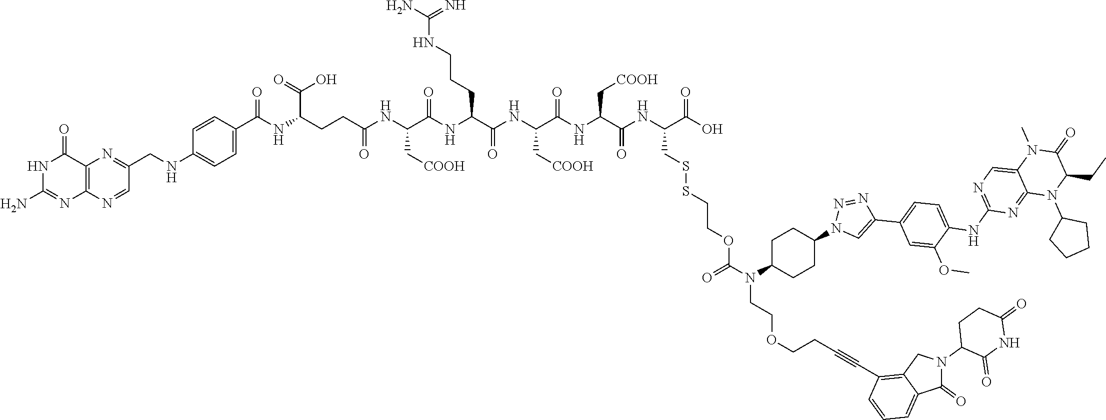 [SMILES: CC[C@@H]1C(=O)N(C)c2cnc(Nc3ccc(-c4cn([C@H]5CC[C@@H](N(CCOCCC#Cc6cccc7c6CN(C6CCC(=O)NC6=O)C7=O)C(=O)OCCSSC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c6ccc(NCc7cnc8nc(N)[nH]c(=O)c8n7)cc6)C(=O)O)C(=O)O)CC5)nn4)cc3OC)nc2N1C1CCCC1] |
| OLW-30 | UB- 181337 | 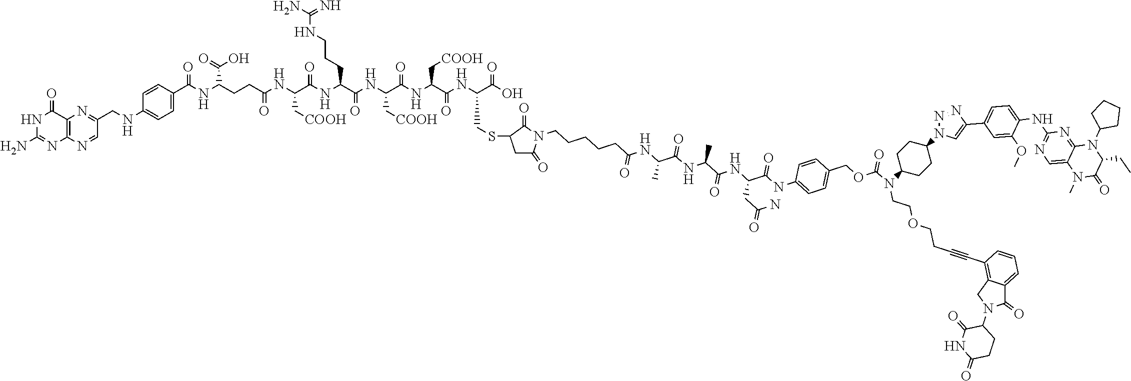 |
| OLW-31 | UB- 181338 | 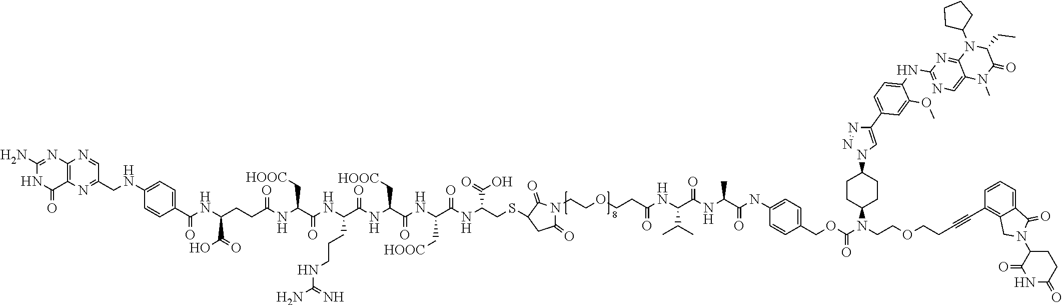 |
| OLW-32 | UB- 181339 | 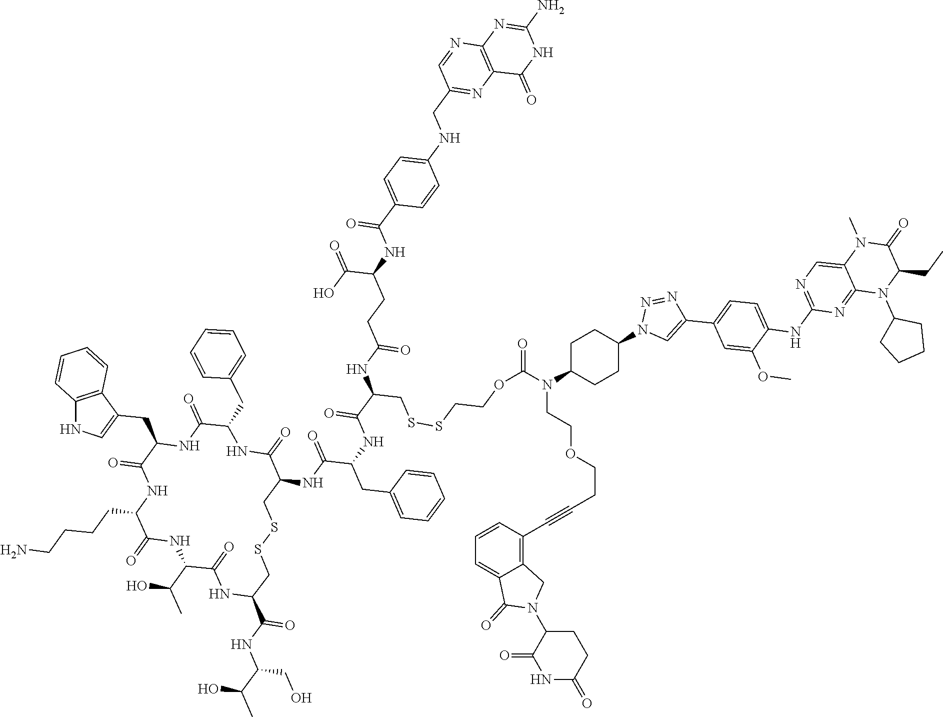 [SMILES: CC[C@@H]1C(=O)N(C)c2cnc(Nc3ccc(-c4cn([C@H]5CC[C@@H](N(CCOCCC#Cc6cccc7c6CN(C6CCC(=O)NC6=O)C7=O)C(=O)OCCSSC[C@H](NC(=O)CC[C@H](NC(=O)c6ccc(NCc7cnc8nc(N)[nH]c(=O)c8n7)cc6)C(=O)O)C(=O)N[C@H](Cc6ccccc6)C(=O)N[C@H]6CSSC[C@@H](C(=O)N[C@H](CO)[C@@H](C)O)NC(=O)[C@H]([C@@H](C)O)NC(=O)[C@H](CCCCN)NC(=O)[C@@H](Cc7c[nH]c8ccccc78)NC(=O)[C@H](Cc7ccccc7)NC6=O)CC5)nn4)cc3OC)nc2N1C1CCCC1] |
| OLW-33 | UB- 181340 | 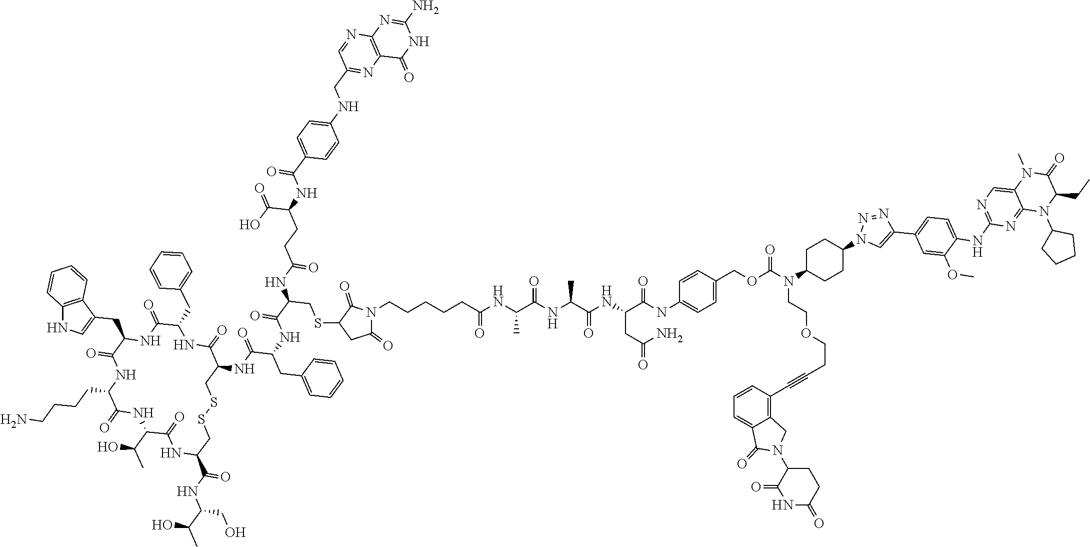 [SMILES: CC[C@@H]1C(=O)N(C)c2cnc(Nc3ccc(-c4cn([C@H]5CC[C@@H](N(CCOCCC#Cc6cccc7c6CN(C6CCC(=O)NC6=O)C7=O)C(=O)OCc6ccc(NC(=O)[C@H](CC(N)=O)NC(=O)[C@H](C)NC(=O)[C@H](C)NC(=O)CCCCCN7C(=O)CC(SC[C@H](NC(=O)CC[C@H](NC(=O)c8ccc(NCc9cnc%10nc(N)[nH]c(=O)c%10n9)cc8)C(=O)O)C(=O)N[C@H](Cc8ccccc8)C(=O)N[C@H]8CSSC[C@@H](C(=O)N[C@H](CO)[C@@H](C)O)NC(=O)[C@H]([C@@H](C)O)NC(=O)[C@H](CCCCN)NC(=O)[C@@H](Cc9c[nH]c%10ccccc9%10)NC(=O)[C@H](Cc9ccccc9)NC8=O)C7=O)cc6)CC5)nn4)cc3OC)nc2N1C1CCCC1] |
| OLW-34 | UB- 181356 | 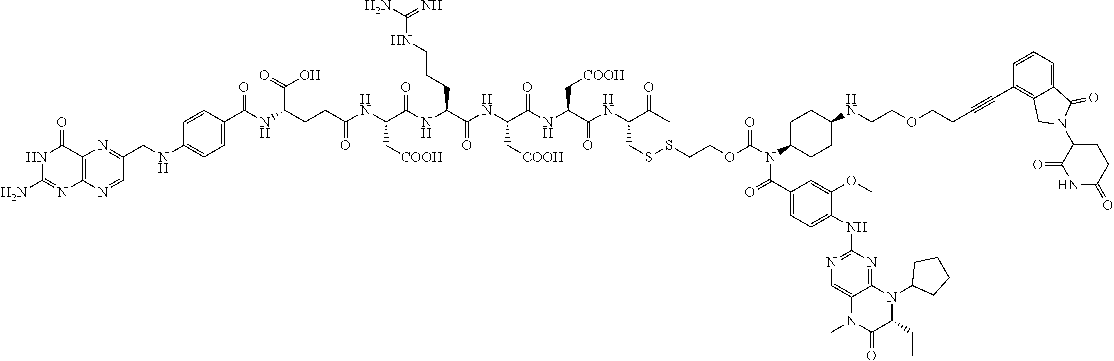 [SMILES: CC[C@@H]1C(=O)N(C)c2cnc(Nc3ccc(C(=O)N(C(=O)OCCSSC[C@H](NC(=O)[C@H](CC(=O)O)NC(=O)[C@H](CC(=O)O)NC(=O)[C@H](CCCNC(=N)N)NC(=O)[C@H](CC(=O)O)NC(=O)CC[C@H](NC(=O)c4ccc(NCc5cnc6nc(N)[nH]c(=O)c6n5)cc4)C(=O)O)C(C)=O)[C@H]4CC[C@@H](NCCOCCC#Cc5cccc6c5CN(C5CCC(=O)NC5=O)C6=O)CC4)cc3OC)nc2N1C1CCCC1] UB- 181356 |
| OLW-35 | UB- 181327 | 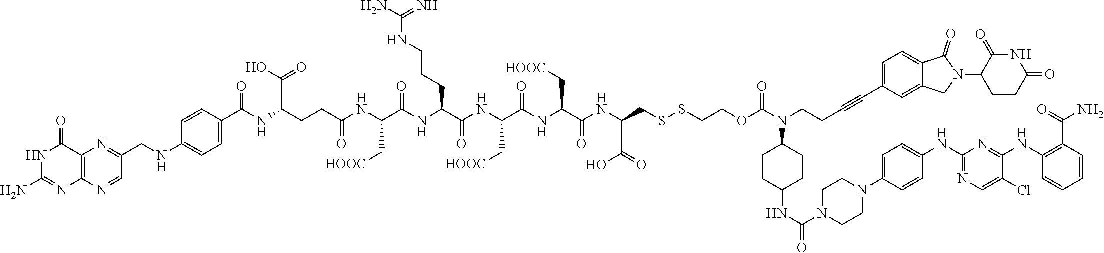 UB- 181327 |
| OLW-36 | UB- 181342 | 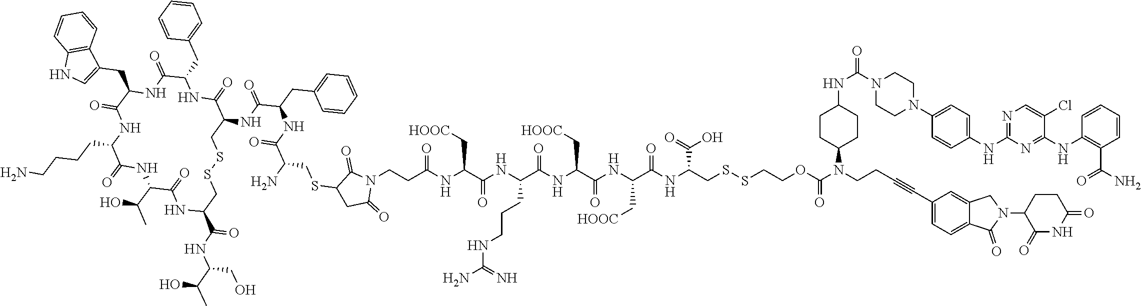 UB- 181342 |
| OLW-37 | UB- 181343 | 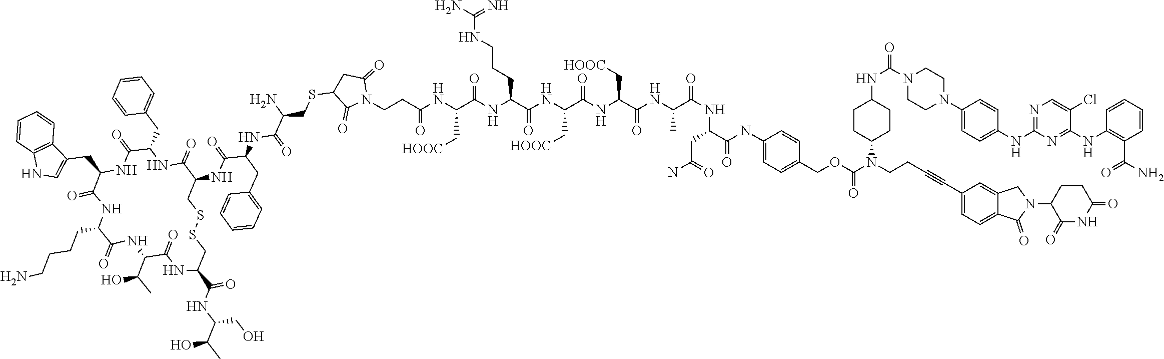 UB- 181343 |
| OLW-38 | UB- 181344 | 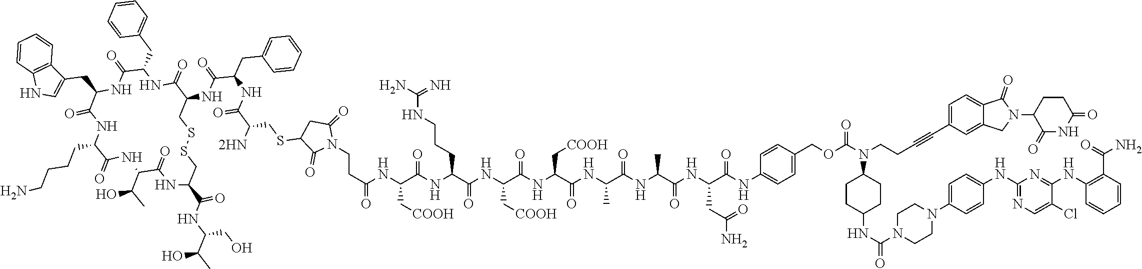 UB- 181344 |
| OLW-39 | UB- 181345 | 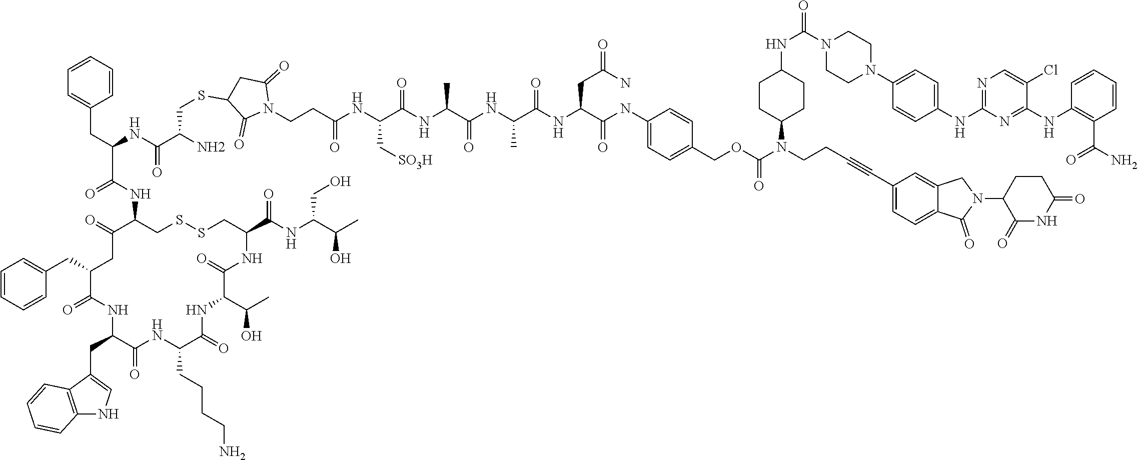 UB- 181345 |
| OLW-40 | UB- 181347 | 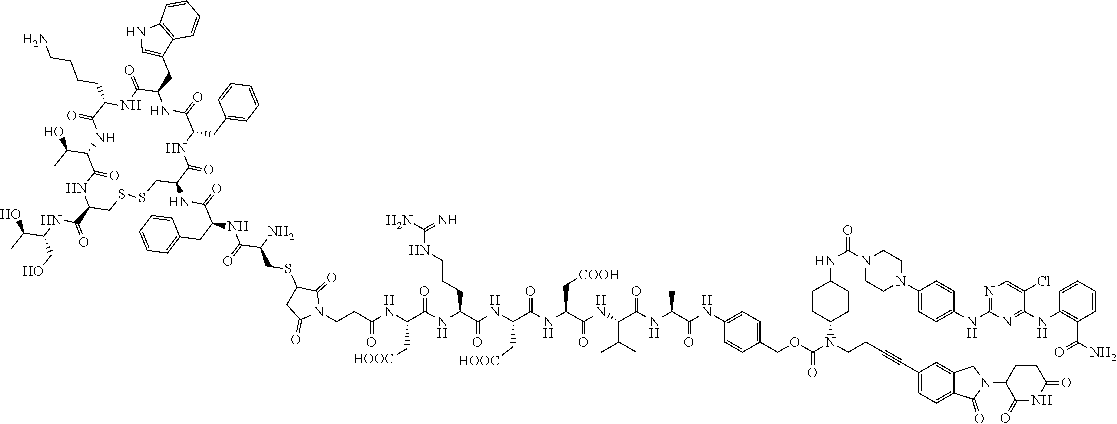 UB- 181347 |
| OLW-41 | UB- 181348 | 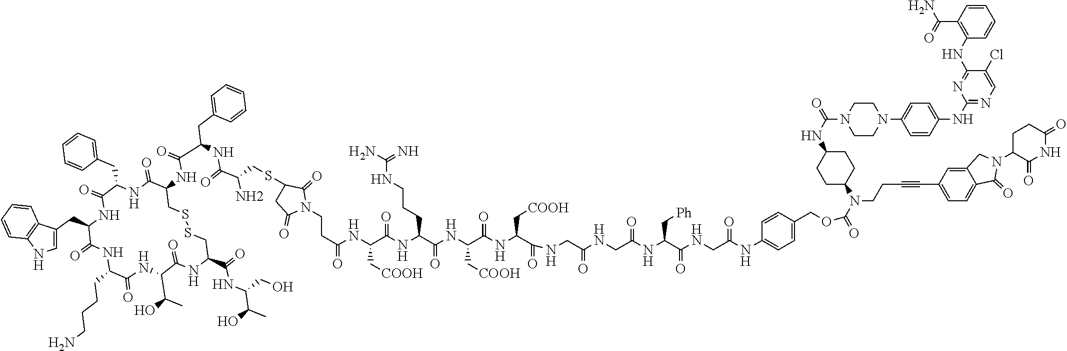 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@@H](Cc2ccccc2)NC(=O)[C@@H](N)CSC2CC(=O)N(CCC(=O)N[C@@H](CC(=O)O)C(=O)N[C@@H](CCCNC(=N)N)C(=O)N[C@@H](CC(=O)O)C(=O)N[C@@H](CC(=O)O)C(=O)NCC(=O)NCC(=O)N[C@@H](Cc3ccccc3)C(=O)NCC(=O)Nc3ccc(COC(=O)N(CCC#Cc4ccc5c(c4)CN(C4CCC(=O)NC4=O)C5=O)[C@H]4CC[C@@H](NC(=O)N5CCN(c6ccc(Nc7ncc(Cl)c(Nc8ccccc8C(N)=O)n7)cc6)CC5)CC4)cc3)C2=O)CSSC[C@@H](C(=O)N[C@H](CO)[C@@H](C)O)NC1=O] UB- 181348 |
| OLW-42 | UB- 181349 | 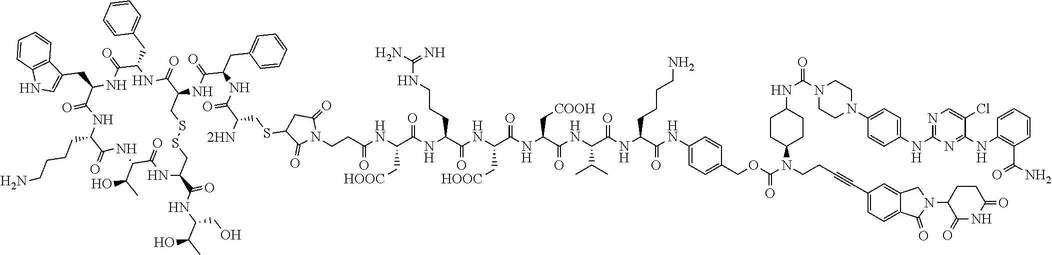 Oct-C-MP-DRDD-VK-PAB-1189 UB- 181349 |
| OLW-43 | UB- 181350 | 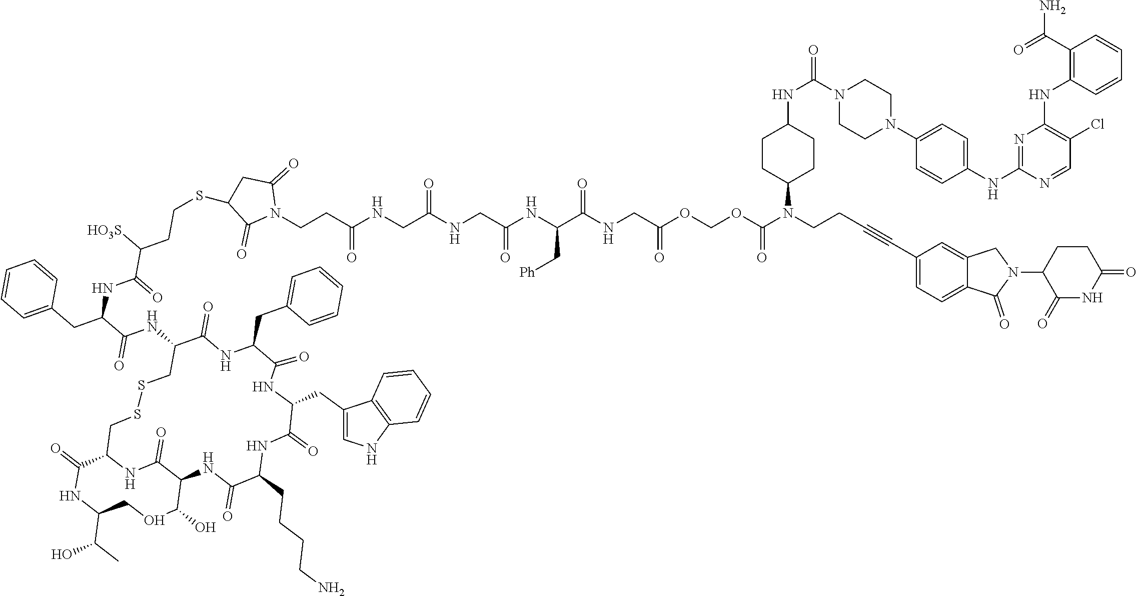 UB- 181350 |
| OLW-44 | UB- 181351 | 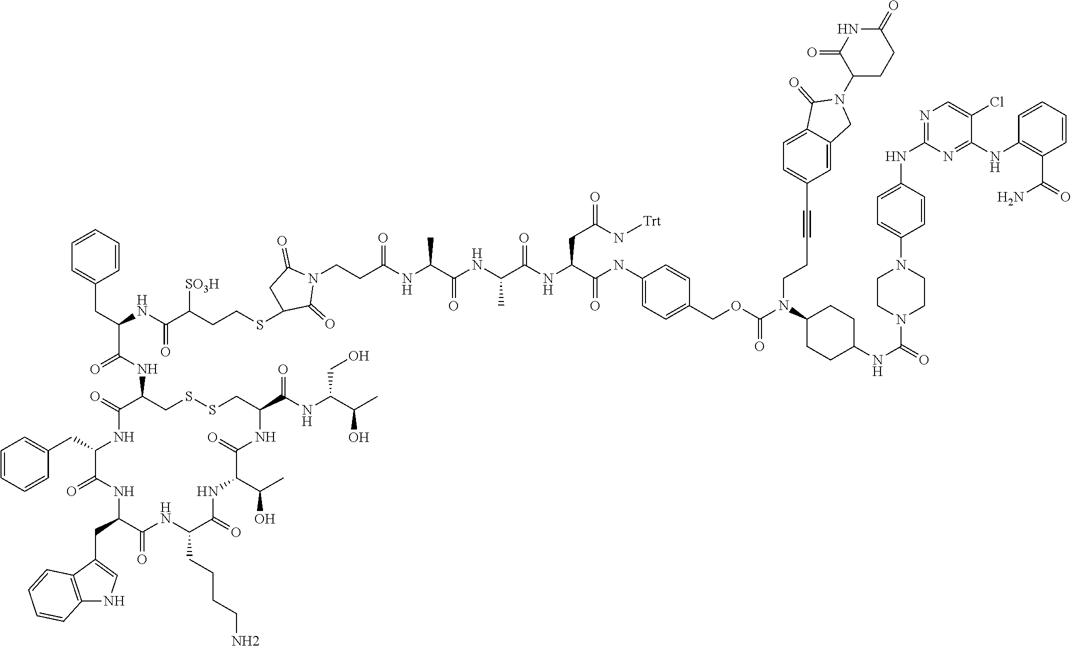 UB- 181351 |
| OLW-45 | UB- 181352 | 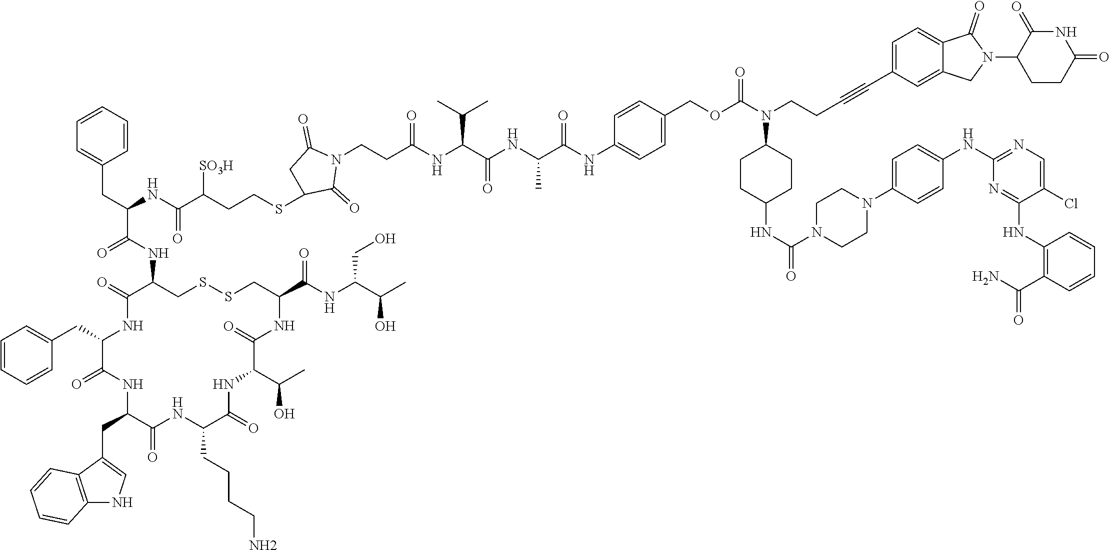 UB- 181352 |
| OLW-46 | UB- 181368 | 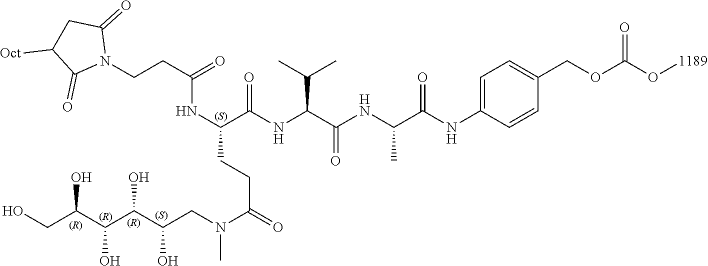 UB- 181368 |
| OLW-47 | UB- 181369 | 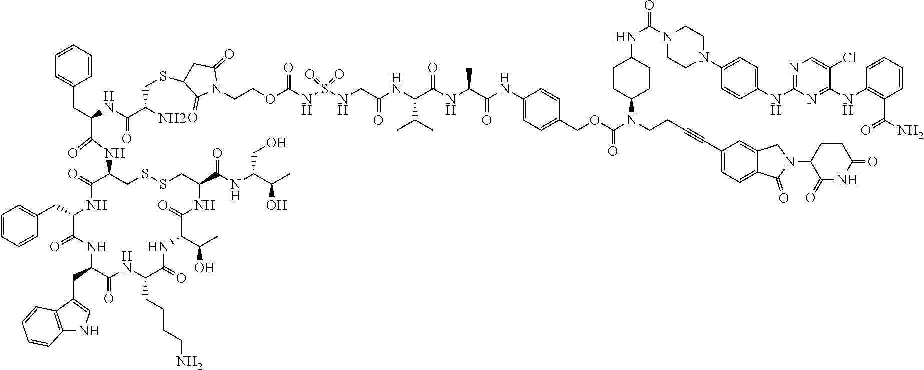 UB- 181369 |
| OLW-48 | UB- 181370 | 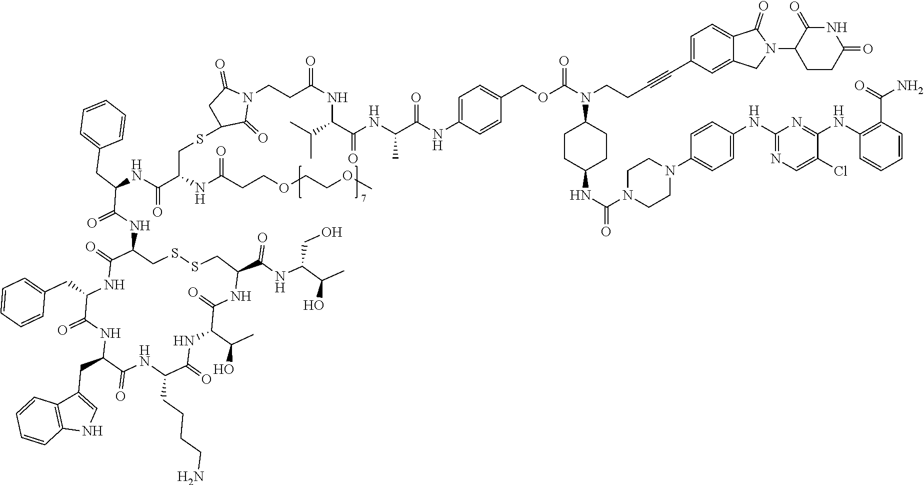 UB- 181370 |
| OLW-49 | UB- 181371 | 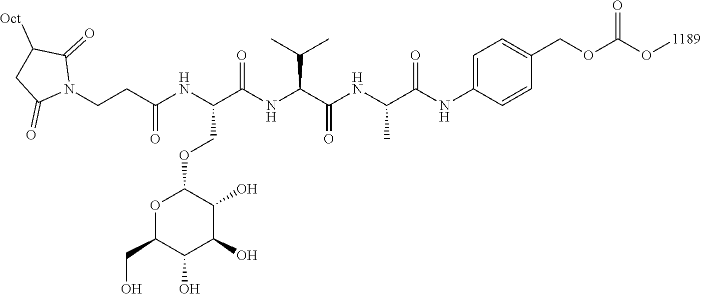 UB- 181371 |
| OLW-50 | UB- 181377 | 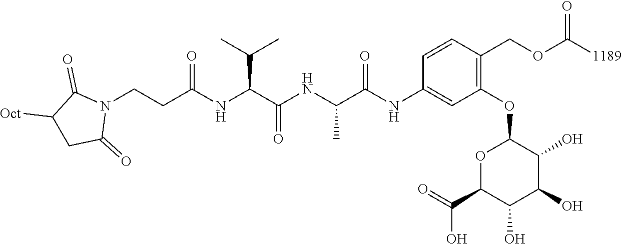 UB- 181377 |
| OLW-51 | UB- 181378 | 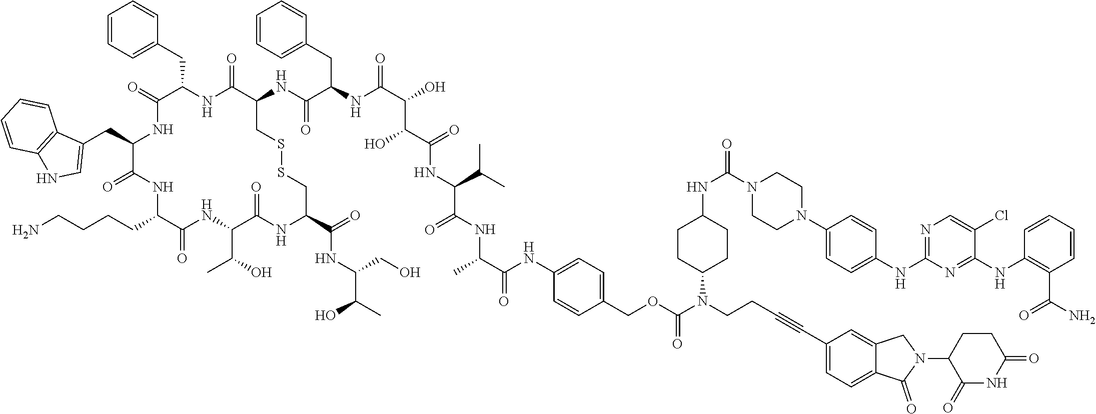 Oct-TA-VA-PAB-1189 UB- 181378 |
| OLW-52 | UB- 181379 | 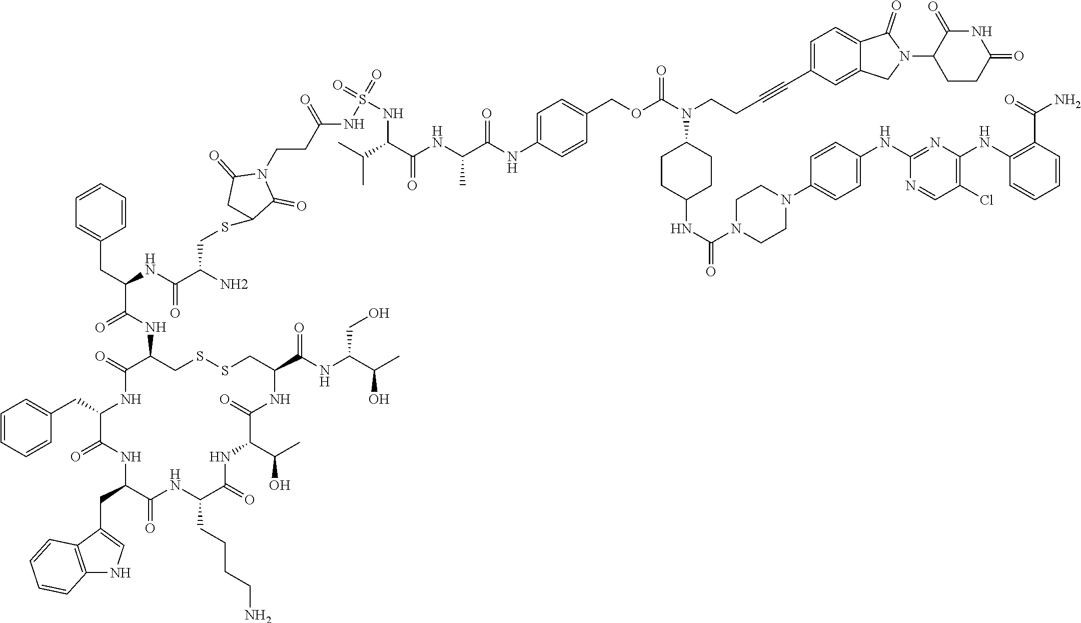 UB- 181379 |
| OLW-53 | UB- 181390 | 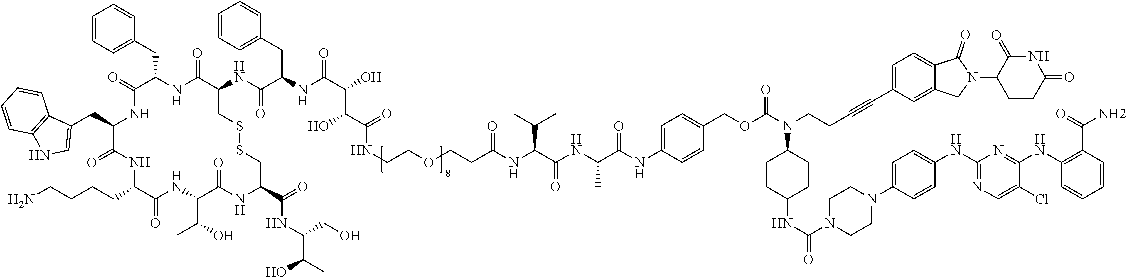 Oct-TA-PEG8-VA-PAB-1189 UB- 181390 |
| OLW-54 | UB- 181393 | 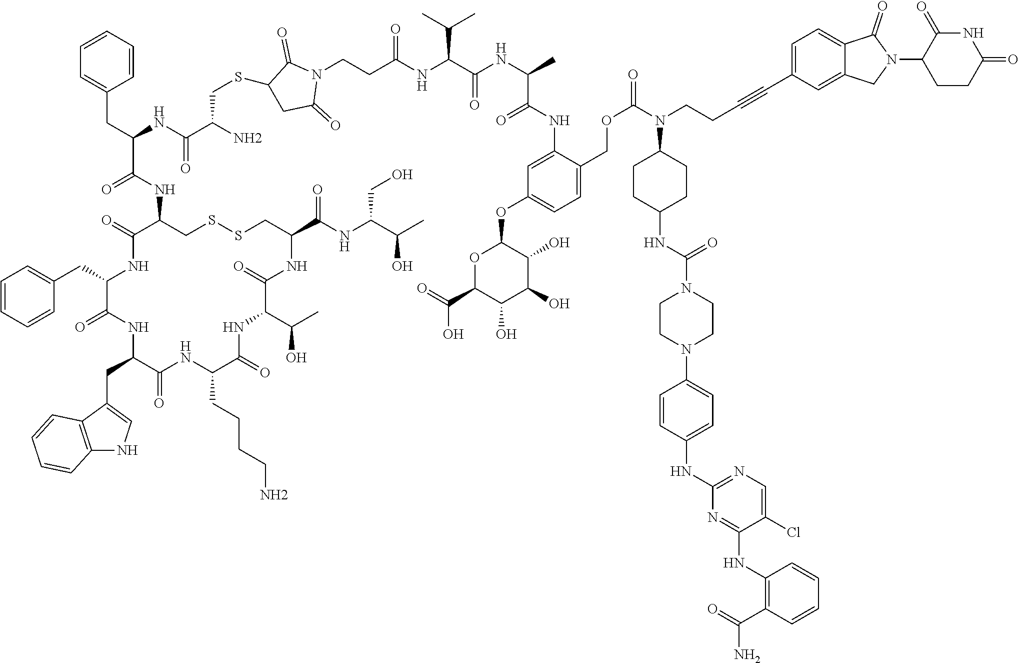 UB- 181393 |

Unless otherwise indicated, in each of the structural formula or reaction schemes in the above preparation examples and in Tables A1, A2, E1, E2, and D, the group indicated by −1189 is a group shown below, wherein * represents the position of attachment to the other portions.

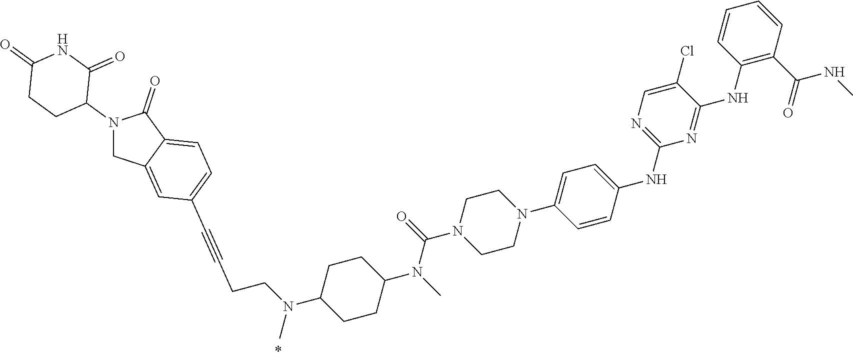 [SMILES: CNC(=O)c1ccccc1Nc1nc(Nc2ccc(N3CCN(C(=O)N(C)C4CCC(N(C)CCC#Cc5ccc6c(c5)CN(C5CCC(=O)NC5=O)C6=O)CC4)CC3)cc2)ncc1Cl]

### TEST EXAMPLE

### Test Example 1 Cell Proliferation Experiment

Reagents: RPMI-1640 medium, McCoy's 5A medium, IMDM medium, MEM medium, L-15 medium, fetal bovine serum, Penicillin-Streptomycin double antibody, trypsin, etc., 2-mercaptoethanol, NEAA, pyruvate, etc.

Some of the cell lines used in this experiment were listed in Table 1 below.

TABLE 1
List of Cell Lines

| No. | Cell Name | Cell Source |
|---|---|---|
| 1 | Human Colon Cancer HT-29 | Shanghai Cell Bank of Chinese |
| | | Academy of Sciences (CAS) |
| 2 | Human Colon Cancer HCT-116 | Shanghai Cell Bank of Chinese |
| | | Academy of Sciences (CAS) |
| 3 | Human Small Cell Lung Cancer | American Type Culture |
| | NCI-H82 | Collection (ATCC) |
| 4 | Human Monocytic Leukemia | Shanghai Cell Bank of Chinese |
| | THP1-1 | Academy of Sciences (CAS) |
| 5 | Human Acute Myelo-monocytic | Shanghai Cell Bank of Chinese |
| | Leukemia HL-60 | Academy of Sciences (CAS) |
| 6 | Human Glioma Cells U-87 MG | Shanghai Cell Bank of Chinese |
| | | Academy of Sciences (CAS) |
| 7 | Human cervical adenocarcinoma | Shanghai Cell Bank of Chinese |
| | cells Hela | Academy of Sciences (CAS) |
| 8 | Human Breast Cancer | Shanghai Cell Bank of Chinese |
| | MDA-MB-231 | Academy of Sciences (CAS) |
| 9 | Human Acute Lymphoblastic | Shanghai Cell Bank of Chinese |
| | Leukemia Cells MOLT-4 | Academy of Sciences (CAS) |
| 10 | Human Ovarian Cancer | Shanghai Cell Bank of Chinese |
| | SK-OV-3 | Academy of Sciences (CAS) |
| 11 | Human Monocytic Leukemia | Shanghai Cell Bank of Chinese |
| | HL-60 | Academy of Sciences (CAS) |
| 12 | Human Monocytic Leukemia | American Type Culture |
| | MV4-11 | Collection (ATCC) |
| 13 | Human umbilical vein | Allcells |
| | endothelial cells HUVEC | |
| 14 | Human peripheral blood mono- | Extracted from peripheral blood |
| | nuclear cells PBMC | of healthy volunteers |
| 15 | Human lymphocytic leukemia | Shanghai Cell Bank of Chinese |
| | cells Daudi | Academy of Sciences (CAS) |
| 16 | Human prostate cancer cells | Shanghai Cell Bank of Chinese |
| | PC-3 | Academy of Sciences (CAS) |
| 17 | human gastric carcinoma cells | |
| | N87 | |
| 18 | Human Colon Cancer cells | |
| | Colo205 | |
| 19 | Human Small Cell Lung Cancer | |
| | H69 | |

The cells were cultured in conventional ways, and the cells should be passed for at least 2 generations before plating. Cells at the logarithmic growth phase were collected, prepared into single cell suspensions and counted. The concentration of cells was adjusted to desired concentration, and cells were inoculated into a 96-well cell culture plate at 100 μl per well. 100 μL of complete medium of the test compound were added to each well, 2 replicate wells for each concentration were set up, and diluted with a 5-fold gradient, and continued to culture for 72 h. All cells were subjected to EC₅₀ test for corresponding samples. The experimental results are shown in Test Example 4.

The fluorescence intensity of each well was detected using the Alarm blue method, and IC₅₀ was calculated.

IC₅₀ was calculated according to the following formula:

$$Y = Max + ( Min - Max ) / [ 1 + ( X / {IC}_{50} ) \times Slope ]$$

wherein Min, Max and Slope represent the minimum, maximum and slope respectively.

### Test Example 2 Western Blot

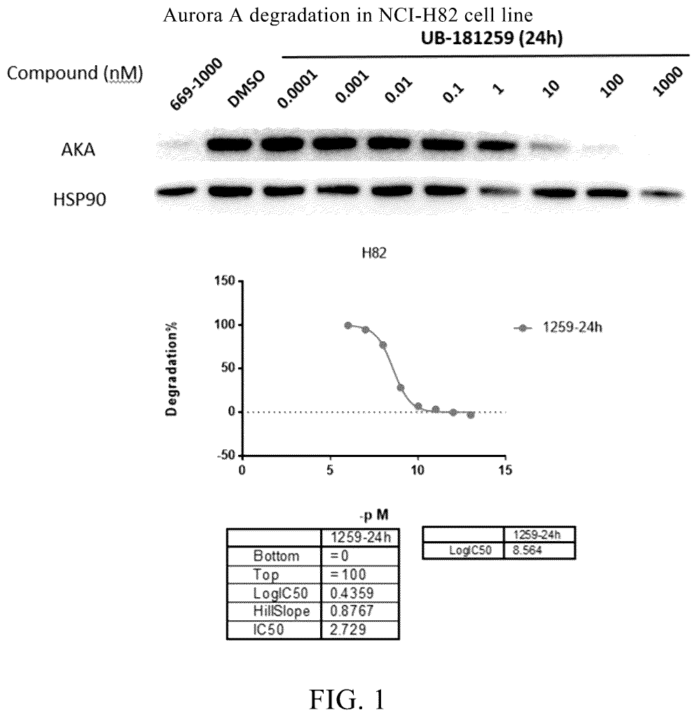

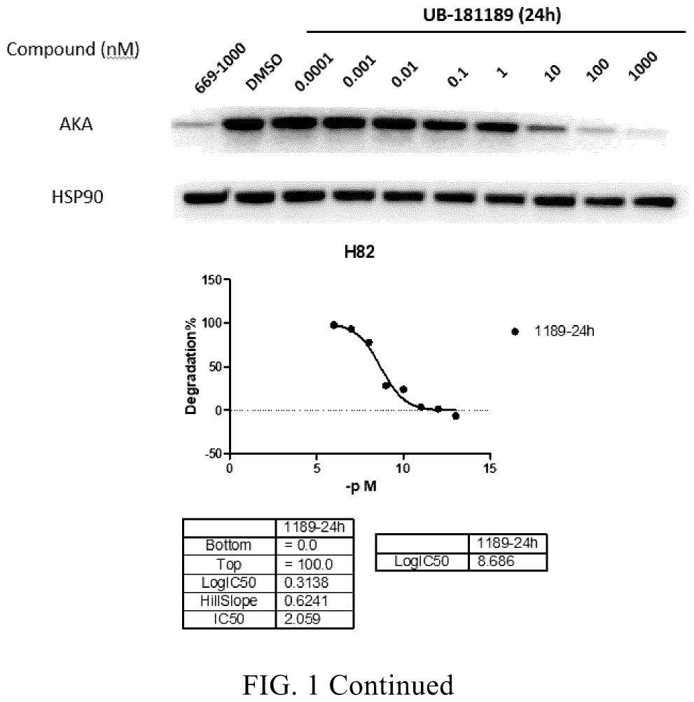

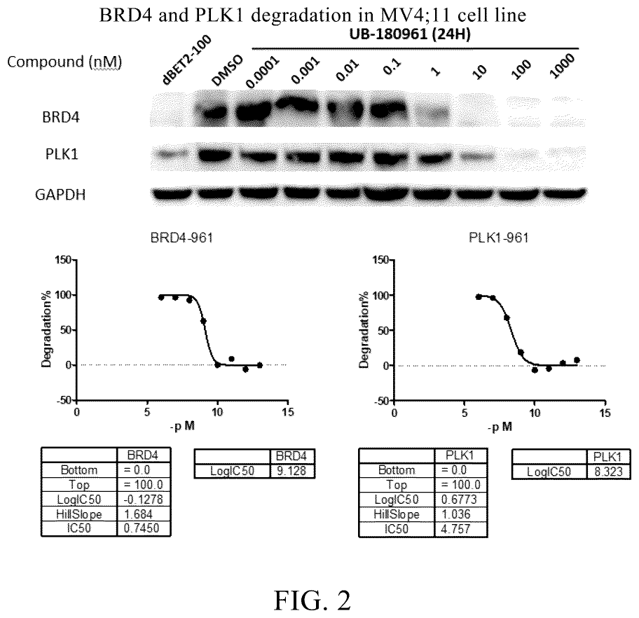

Cells were treated with the compound for a period of time, after that the cells were collected by centrifugation. After washing with PBS, the cells were lysed by adding RIPA buffer; the cell lysate were added to the loading buffer and then appropriate volume were taken and slowly added to the corresponding wells of the gel plate, and run the SDS-PAGE gel (4%-12%). After running the gel, transferred to the PVDF membrane and sealed with 5% skimmed milk powder at room temperature for 1 hour. The membrane was placed in the primary antibody diluted with 5% skimmed milk powder and shook slowly overnight at 4° C. After incubation with primary antibodies, the membrane was washed 3 times using a TBST shaker; added with secondary antibodies diluted with 5% skimmed milk powder corresponding to the primary antibodies, and shook slowly at room temperature for 1 hour. After incubation with secondary antibodies, the membrane was washed 3 times using the TBST shaker again. The PVDF membrane was flatted in the cassette, the strip was evenly infiltrated with ECL developer solution, and placed in ChemDoc XRS+ gel imager for taking pictures. The intensity of protein bands was analyzed quantitatively using ImageJ software. The results were shown in FIG. 1 and FIG. 2.

It can be seen that the conjugate (or TED molecules) of the present invention exhibit concentration-dependent degradation activity against the target protein.

### Test Example 3 In Vitro Kinase Activity Test

The compound, enzyme, substrate and ATP were diluted using 1× reaction buffer to desired concentration. 1 μL of compounds with different concentrations, 2 μL of enzyme, and 2 μL of substrate/ATP mixed solution were added to the 384-well plate, and incubated for 1 hour at room temperature. Then 5 μL of ADP1-Glo™ reagent was added to each well and incubated at room temperature for 40 minutes. Finally, 10 μL of detection reagents were added, and incubated at room temperature for 30 minutes, and chemiluminescence signals were detected using Envision.

It can be seen that the TED molecule synthesized and prepared in the present invention exhibits strong cell proliferation inhibitory activity in a variety of tumor cell lines and has the prospect of becoming an antitumor drug.

### Test Example 4

The activity of part of the compounds (or conjugates) in Table A1 was tested according to the method of the above-mentioned test examples, and the test results are summarized in Table 2

TABLE 2

| | Anti cell proliferative activity EC50 (nM) | | | | |
|---|---|---|---|---|---|
| UB No. | H82 | N87 | Colo205 | H69 | HT-29 |
| UB-181235 | 252.9 | 812.8 | 143.9 | >1000 | — |
| UB-181236 | 602.6 | 92.5 | 145.9 | >1000 | — |
| UB-181237 | 141.3 | 308.3 | 277.3 | >1000 | 901.6 |
| UB-181238 | >1000 | >1000 | — | — | — |
| UB-181239 | 354.0 | 131.8 | 175.0 | >1000 | — |
| UB-181240 | 37.0 | 92.0 | 40.1 | >1000 | 180.7 |
| UB-181242 | 94.2 | 225.9 | 75.2 | >1000 | >1000 |
| UB-181243 | 239.9 | 668.3 | 311.2 | >1000 | >1000 |
| UB-181246 | 298.5 | 515.2 | 236.0 | >1000 | >1000 |
| UB-181247 | 91.4 | 168.3 | 169.4 | >1000 | >1000 |
| UB-181249 | 385.5 | 66.1 | 52.0 | >1000 | — |
| UB-181250 | 53.7 | 20.5 | 48.2 | >1000 | — |
| UB-181251 | 32.4 | 71.9 | 133.0 | >1000 | — |
| UB-181257 | 42.3 | 71.8 | 59.4 | >1000 | — |
| UB-181258 | 23.8 | 24.6 | 37.7 | 31.3 | 400.9 |
| UB-181259 | 20.9 | 111.4 | 33.9 | 19.1 | 193.6 |
| UB-181261 | 24.9 | 23.6 | 24.8 | 61.2 | — |
| UB-181263 | 53.0 | >1000 | 9.1 | 192.3 | >1000 |
| UB-181265 | 912.0 | >1000 | 3.8 | >1000 | >1000 |
| UB-181266 | 151.7 | >1000 | 233.9 | >1000 | >1000 |
| UB-181267 | 168.3 | >1000 | 518.8 | >1000 | >1000 |
| UB-181268 | >1000 | >1000 | >1000 | >1000 | >1000 |
| UB-181269 | 11.6 | 28.8 | 10.7 | >1000 | 35.5 |
| UB-181270 | 146.6 | 257.6 | 218.3 | 453.9 | 950.6 |
| UB-181272 | 19.1 | 23.1 | 21.1 | >1000 | 331.9 |
| UB-181273 | 21.6 | >1000 | 21.4 | 375.0 | 56.2 |
| UB-181274 | 27.7 | 18.2 | 49.4 | >1000 | 522.4 |
| UB-181275 | 739.6 | >1000 | 441.6 | >1000 | 968.3 |
| UB-181279 | 24.9 | >1000 | 65.2 | >1000 | 415.0 |
| UB-181280 | 647.1 | >1000 | 1.9 | >1000 | >1000 |
| UB-181285 | 125.0 | 50.0 | 62.1 | >1000 | 528.4 |
| UB-181289 | 410.2 | 698.2 | 620.9 | >1000 | >1000 |
| UB-181290 | insoluble | insoluble | insoluble | insoluble | insoluble |
| UB-181291 | 166.0 | >1000 | 92.5 | >1000 | >1000 |
| UB-181294 | 244.9 | 818.5 | 301.3 | >1000 | 865.0 |
| UB-181295 | 106.4 | — | 136.1 | 952.8 | 484.2 |
| UB-181297 | >1000 | >1000 | >1000 | >1000 | >1000 |
| UB-181298 | 8.8 | 272.9 | 4.1 | >1000 | >1000 |
| UB-181299 | 245.5 | 679.2 | 302.0 | >1000 | >1000 |
| UB-181301 | 43.8 | 14.3 | 105.7 | >1000 | 452.9 |
| UB-181303 | 471.0 | >1000 | >1000 | >1000 | >1000 |
| UB-181308 | 62.4 | 54.2 | 100.0 | >1000 | 721.1 |
| UB-181309 | — | — | — | — | — |
| UB-181310 | 247.7 | 907.8 | 571.5 | >1000 | >1000 |
| UB-181311 | 270.4 | >1000 | 574.1 | >1000 | >1000 |
| UB-181313 | — | — | — | — | — |
| UB-181315 | 314.1 | >1000 | 722.8 | >1000 | >1000 |
| UB-181321 | 171.4 | >1000 | 238.8 | >1000 | >1000 |
| UB-181322 | 311.9 | 469.9 | 383.7 | >1000 | >1000 |
| UB-181327 | 283.1 | >1000 | 242.7 | >1000 | 855.1 |
| UB-181334 | 988.6 | 926.8 | 285.8 | 955.0 | >1000 |
| UB-181335 | >1000 | >1000 | >1000 | >1000 | >1000 |
| UB-181336 | >1000 | >1000 | >1000 | >1000 | >1000 |
| UB-181337 | 183.2 | 175.4 | 33.1 | 79.3 | 232.8 |
| UB-181339 | 236.0 | 456.0 | 248.9 | >1000 | >1000 |
| UB-181342 | 746.4 | >1000 | 847.2 | >1000 | >1000 |
| UB-181343 | 103.0 | 304.8 | 285.1 | 737.9 | >1000 |
| UB-181344 | 398.1 | 645.7 | 301.3 | >1000 | >1000 |
| UB-181345 | 92.9 | 299.9 | 274.2 | >1000 | >1000 |
| UB-181347 | 41.1 | 180.7 | 153.1 | 189.7 | 671.4 |

### Test Example 5: Evaluation of the In Vivo Efficacy of UB-181322 in the Nude Mouse Xenograft Tumor Model of Human Small Cell Lung Cancer NCI-H82

Transplanted tumor cell line: human small cell lung cancer cell NCI-H82, derived from the American Type Culture Collection Center (ATCC, cryopreserved in liquid nitrogen in the laboratory). Under 5% CO₂ and 37° C. culture conditions, cells were routinely cultured in RPMI-1640 culture medium containing 10% fetal bovine serum; they were passaged according to cell growth, with a passage ratio of 1:2 to 1:5.

Experimental animal: Female BALB/c nude mice (number: 65; age: 6-8 weeks) were purchased from Charles River and raised in the SPF animal room of Suzhou Shengsu New Pharmaceutical Development Co., Ltd. (3D Biooptima). The temperature is 20 to 25° C., and relative humidity is 40% to 70%, with bright light and dark light for 12 hours each; animals were free to drink and eat. After normal feeding for about 5 days, mice with good physical signs can be selected for this experiment after veterinary inspection. Before grouping, animals were identified at the base of tail using maker pens. After grouping, each animal was identified by ear clipping.

Animal model preparation: NCI-H82 cells at logarithmic growth phase were collected, the cells were resuspended in 50% serum-free RPMI-1640 medium and 50% Matrigel after counting. The concentration of cells was adjusted to 1.5×10′ cells/mL. The cells were dispersed evenly by blowing and beating using pipette and placed into a 50-mL centrifuge tube, and the centrifuge tube was placed in ice box. The cell suspension was suck up using 1 mL syringe, and injected into the Right anterior axillary subcutaneous of nude mice. Each animal was inoculated with 200 μL (3.0×10⁶ cells/mouse) to establish NCI-H82 xenograft tumor model. After inoculation, the animal status and tumor growth were regularly observed, the tumor diameter was measured using a electronic vernier caliper, the data were entered into Excel to calculate the tumor volume. When the Tumour volume reached 100-300 mm³, animals in good health and with similar tumor volumes were selected, and separated into 10 groups (n=4) using randomized block method. The day of grouping was the first day of the experiment (D1). After the start of experiment, the tumor diameter was measured twice a week, the tumor volume was calculated, and the animal weight was weighed and recorded at the same time.

Calculation formula for tumor volume (TV) is as follows:

$$TV ( {mm}^{3} ) = l \times {w}^{2} / 2$$

wherein, l means the long diameter of the tumor (mm); w means the short diameter (mm) of the tumor.

### Preparation of Dosing Preparations

Preparation of blank solvent: Appropriate volumes of DMSO, 30% Solutol aqueous solution and ultrapure water were taken respectively, and mixed evenly, used as blank solvent, and stored at room temperature for later use. The proportions of DMSO, 30% Solutol aqueous solution and ultrapure water in the mixed solvent are 5%, 20% and 75%.

Preparation of UB-181322 sample: An appropriate amount of UB series sample was weighed and put it into a glass bottle; an appropriate volume of DMSO was added. After vortexing to make the compound being completely dissolved, a stock solution was prepared, subpackaged and stored in a −20° C. refrigerator. Before each administration, one tube was taken and added with an appropriate volume of 30% Solutol aqueous solution, vortexed to mix well, and finally added with an appropriate volume of ultrapure water. The liquid was vortexed to mix evenly, with the proportions of DMSO, 30% Solutol aqueous solution and ultrapure water in the liquid being 5%, 20% and 75%, to obtain preparations with appropriate final concentrations, which are ready for use.

### Data Record, Calculation Formula

Calculation formula for the relative tumor volume (RTV) is:

$$RTV = {TV}_{t} / {TV}_{initial}$$

wherein TVᵢₙᵢₜᵢₐₗ is the tumor volume measured at grouped administration; TVₜ is the tumor volume at each measurement during the administration period.

Calculation formula for the relative tumor proliferation rate (% T/C) is:

$$\% T / C = 100 \% \times ( {RTV}_{T} / {RTV}_{C} )$$

wherein RTV_{T} means the RTV of treatment group; RTV_{C} means the RTV of solvent control group.

Calculation formula for tumor growth inhibition rate TGI (%) is:

$$TGI ( \% ) = 100 \% \times [ 1 - ( {TV}_{t ( T )} - {TV}_{initial ( T )} ) / ( {TV}_{t ( C )} - {TV}_{initial ( C )} ) ]$$

wherein TV_{t(T)} means the tumor volume of the treatment group at each measurement; TV_{initial(T)} means the tumor volume of the treatment group at grouped administration; TV_{t(C)} means the tumor volume of solvent control group at each measurement; TV_{initial(C)} means the tumor volume of solvent control group at grouped administration.

Calculation formula for the relative body weight (RBW) of animal (%) is:

$$RBW = 1 \; 00 \times {BW}_{t} / {BW}_{initial}$$

wherein BWᵢₙᵢₜᵢₐₗ is the animal body weight at grouped administration; BWₜ is the animal body weight at each measurement during the administration period.

Calculation formula for the loss rate of animal weight is:

$$Loss rate of animal weight = 100 \% \times ( {BW}_{initial} - {BW}_{t} ) / {BW}_{initial}$$

wherein, BWₜ means the animal weight at each measurement during administration; BWᵢₙᵢₜᵢₐₗ means the animal weight at grouped administration.

Calculation formula for the inhibition rate of tumor weight IR (%) is:

$$IR ( \% ) = 100 \% \times ( {W}_{C} - {W}_{T} ) / {W}_{C}$$

wherein W_{C} means the tumor weight of control group; W_{T} means the tumor weight of treatment group.

Statistical analysis method: the experimental data were calculated and performed related statistical processing using Microsoft Office Excel 2007 software. Unless otherwise stated, data were expressed as mean±SE, and t-test was used for comparison between two groups.

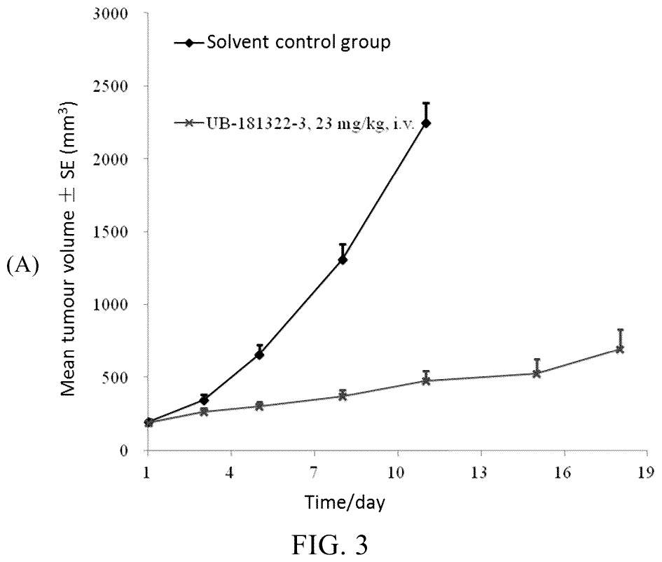

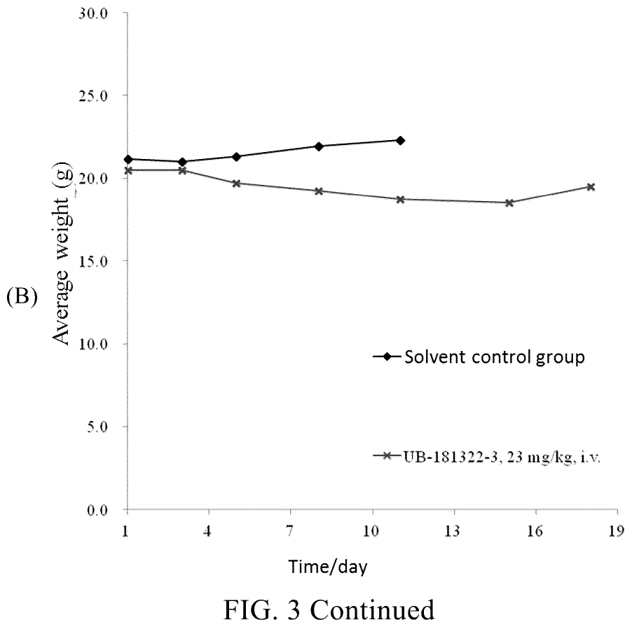

The results were shown in FIG. 3, it can be seen that compared with blank group, UB-181322 shows the effect of inhibiting tumor growth (A), and the mouse body weight does not change significantly during the administration period, and the toxicity was low (B).

All documents mentioned in the present invention are cited as references in this application, just as each document is individually cited as a reference. In addition, it should be understood that, after reading the above teaching content of the present invention, those skilled in the art can make various changes or modifications to the present invention, and these equivalent forms also fall within the scope defined by the appended claims of the present application.

1. A conjugate of formula I, or a pharmaceutically acceptable salt thereof, wherein
R_{T}-L1-R_{E3} (I)
wherein
(a) R_{E3} is a moiety of E3 Ligase Ligand;
(b) R_{T} is a moiety of target molecule;
(c) L1 is a linker connecting the moieties of R_{E3} and R_{T}, and L1 is as shown in formula II;
—W¹-L2-W²— (II)
wherein
W¹ and W² are each independently —(W)ₛ—;
W is each independently selected from the group consisting of: null(bond), —C(R^{b})₂—, —O—, —S—, —N(R^{a})—, —C(O)—, —SO₂—, —SO—, —PO₃', —C(R^{b})═C(R^{b})—, —C≡C—, NR, substituted or unsubstituted C3-8 cycloalkyl, substituted or unsubstituted 4 to 10 membered heterocycloalkyl, substituted or unsubstituted C6-10 aryl, substituted or unsubstituted 5 to 10 membered heteroaryl;
s=0, 1, 2, 3, or 4;
L2 is of formula III,
-(M^{L})ₒ- (III)
wherein
M^{L} is each independently M, M^{T} or M^{N};
wherein
is an integer selected from 5 to 50;
M is each independently a divalent group selected from the group consisting of: —C(R^{b})₂—, —O—, —S—, —N(R^{a})—, —C(O)—, —SO₂—, —SO—, —PO₃—, —C(R^{b})═C(R^{b})—, —C≡C—, substituted or unsubstituted C₃₋₈ cycloalkyl, substituted or unsubstituted 4 to 10 membered heterocycloalkyl, substituted or unsubstituted C₆₋₁₀ aryl, substituted or unsubstituted 5 to 10 membered heteroaryl, and amino acid residue;
M^{N} is each independently a divalent group selected from the group consisting of: —N(R′)—, —N(4 to 10 membered heterocycloalkyl containing N(R′) as ring atom)-, 4 to 10 membered heterocycloalkyl containing N(R′) as ring atom, —C(R^{b})₂— substituted with at least one —N(R^{b})R′ (preferably, —NHR′), C₃₋₈ cycloalkyl, 4 to 10 membered heterocycloalkyl, C₆₋₁₀ aryl, and 5 to 10 membered heteroaryl;
M^{T} is each independently a divalent group selected from the group consisting of: —N(R″)—, —N(4 to 10 membered heterocycloalkyl containing N(R″) as ring atom)-, 4 to 10 membered heterocycloalkyl containing N(R″) as ring atom, —C(R^{b})₂— substituted with at least one —N(R^{b})R″, C₃₋₈ cycloalkyl, 4 to 10 membered heterocycloalkyl, C₆₋₁₀ aryl, and 5 to 10 membered heteroaryl;
R is R′ or R″;
R′ is each independently selected from the group consisting of H, C₁₋₆ alkyl, OH, SH, —COO—C₁₋₆ alkyl, —OC(O)—C₁₋₆alkyl, and amino protecting group;
R″ is —W³-L^{T1}-W^{P1}—(R_{P})_{q1};
subscript q1>0 (preferably, q1=1);
W^{P} is null, —S—S— or 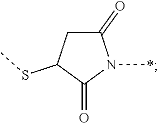
wherein * indicates the part connected with L^{T1}; preferably W^{P1} is —S—S— or 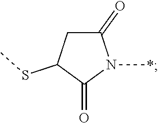
R_{P} is —W⁴—R_{P1}; W⁴ is null or —(W″)ₛ₁—W^{P2}—(W″)ₛ₂—; wherein subscript s1 and s2 are each independently 0, 1, 2, 3 or 4, W^{P2} is null, NH, —C(R^{b})(NR^{a})— (such as —CH(—NH₂)—), —N(R′″)— or —C(R^{b})(NH(R′″))—;
R′″ is —W⁵-L^{T2}-W⁶-L^{T3}-R_{P2};
L^{T1} is -(M′)^{t1}—W_{Y}-(M′)ₜ₂-;
L^{T2} is -(M′)ₜ₃-;
L^{T3} is -(M′)ₜ₄-;
subscripts t1, t2, t3 and t4 are each independently 0, 1, 2, 3, 4, 5, 6, 7, 8, 9 or 10 (preferably, t1, t2, t3 and t4 are each independently 0, 1, 2 or 3);
M′ is each independently selected from the group consisting of: —C(R^{b})₂—, —O—, —S—, —N(R^{a}), —C(O)—, —SO₂—, —SO—, —PO₃—, substituted or unsubstituted C1-10alkylene, —(CH₂CH₂O)₁₋₁₀—, amino acid residue, substituted or unsubstituted C3-8cycloalkyl, substituted or unsubstituted 4-10 membered heterocycloalkyl, substituted or unsubstituted C6-10aryl, and substituted or unsubstituted 5-10 membered heteroaryl; and any one or two M′ is W_{X};
W_{X} is a moiety of hydrophilic bivalent linker;
W_{Y} is null or a moiety of bivalent linker that is cleavable at the cell surface or in the cytoplasm;
W³ is —(W′)ₛ₃—; wherein subscript s3=0, 1 or 2;
W⁵ is —(W′)ₛ₄—; wherein subscript s4=0, 1 or 2;
W⁶ is 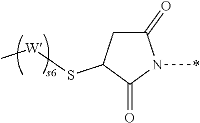
or —(W″)ₛ₆—; wherein subscript s6=0, 1, 2, 3 or 4;
W′ are each independently a divalent group selected from the group consisting of: —C(R^{b})₂—, —O—, —S—, —N(R^{a})—, —C(O)—, —SO₂—, —SO—, —PO₃—, amino acid residue, substituted or unsubstituted C3-8 cycloalkyl, substituted or unsubstituted 4 to 10 membered heterocycloalkyl, substituted or unsubstituted C6-10 aryl, and substituted or unsubstituted 5 to 10 membered heteroaryl;
W″ are each independently a divalent group selected from the group consisting of: —C(R^{b})₂—, —O—, —S—, —N(R^{a})—, —C(O)—, —SO₂—, —SO—, —PO₃—, amino acid residue, substituted or unsubstituted C3-8 cycloalkyl, substituted or unsubstituted 4 to 10 membered heterocycloalkyl, substituted or unsubstituted C6-10 aryl, and substituted or unsubstituted 5 to 10 membered heteroaryl;
R_{P1} and R_{P2} are each independently the same or different polypeptide element or target molecule T; preferably, R_{P1} and R_{P2} are each independently different polypeptide element or target molecule T;
R^{a} is each independently selected from the group consisting of: H, OH, SH, substituted or unsubstituted C₁₋₆alkyl, amino protecting group, 4 to 10 membered heterocycloalkyl containing N(R^{c}) as ring atom;
R^{b} is each independently selected from the group consisting of: H, halogen, OH, SH, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₁₋₆ alkanoyl (—C(O)—C₁₋₆alkyl), carboxyl, —COO—C₁₋₆alkyl, —OC(O)—C₁₋₆ alkyl; or, two R^{b} on the same atom together with the carbon to which they are attached form a substituted or unsubstituted C₃₋₈cycloalkyl, substituted or unsubstituted 4 to 10 membered heterocycloalkyl;
R^{c} is each independently selected from the group consisting of: H, OH, SH, substituted or unsubstituted C₁₋₆alkyl, and amino protecting group;
unless otherwise specified, said substituted means that one or more (such 1, 2, or 3) hydrogen atoms in the group are substituted with substituents selected from the group consisting of: halogen (preferably, F, Cl, Br or I), cyano(CN), oxo (═O), thio (═S), C₁₋₆alkyl, C₁₋₆haloalkyl, C₂₋₆alkenyl, C₂₋₆alkynyl, C₁₋₆alkoxy, C₁₋₆alkanoyl (C₁₋₆alkyl-C(O)—), —COO—C₁₋₆alkyl, —OC(O)—C₁₋₆alkyl, NH₂, NH(C₁₋₆alkyl), N(C₁₋₆alkyl)₂.

2. The conjugate of claim 1 or the pharmaceutically acceptable salt thereof, wherein,
W is not NR; and
L2 is L7, and L7 is of formula IIIb;
-(M)ₒ₁-(M^{T})-(M)ₒ₂- (IIIb)
wherein M, and M^{T} are defined as above;
o1 and o2 are each independently integers selected from 1 to 50, and 4≤o1+o2≤49.

3. The conjugate of claim 1 or the pharmaceutically acceptable salt thereof, wherein the moiety of bivalent linker that is cleavable at the cell surface or in the cytoplasm is a bivalent linker moiety composed of two or more structural fragments selected from the group consisting of:

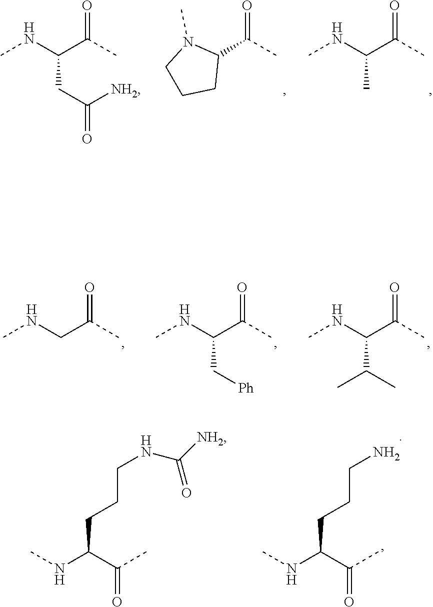

4. The conjugate of claim 1 or the pharmaceutically acceptable salt thereof, wherein
the moiety of bivalent linker that is cleavable at the cell surface or in the cytoplasm is selected from the group consisting of: 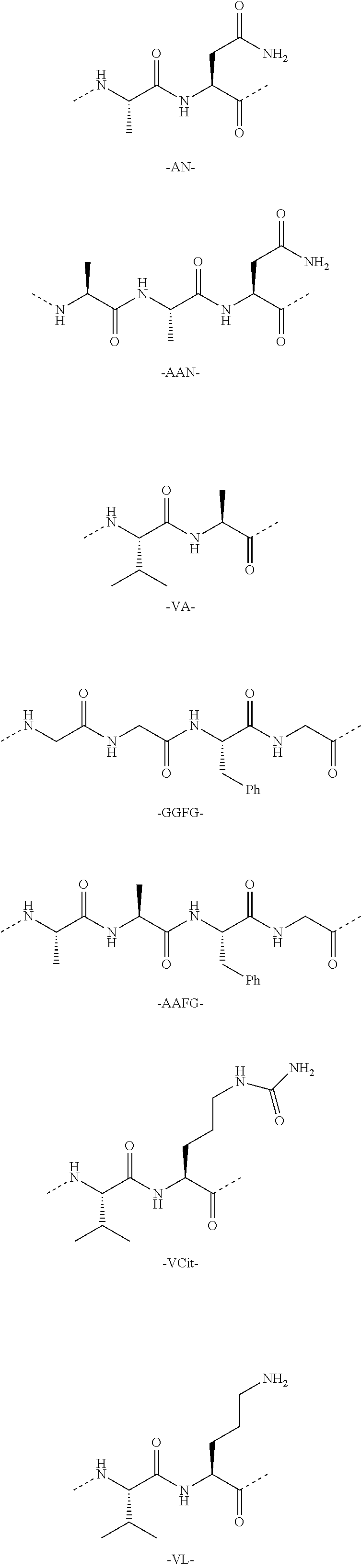
and/or,
the moiety of hydrophilic bivalent linker is selected from the group consisting of: 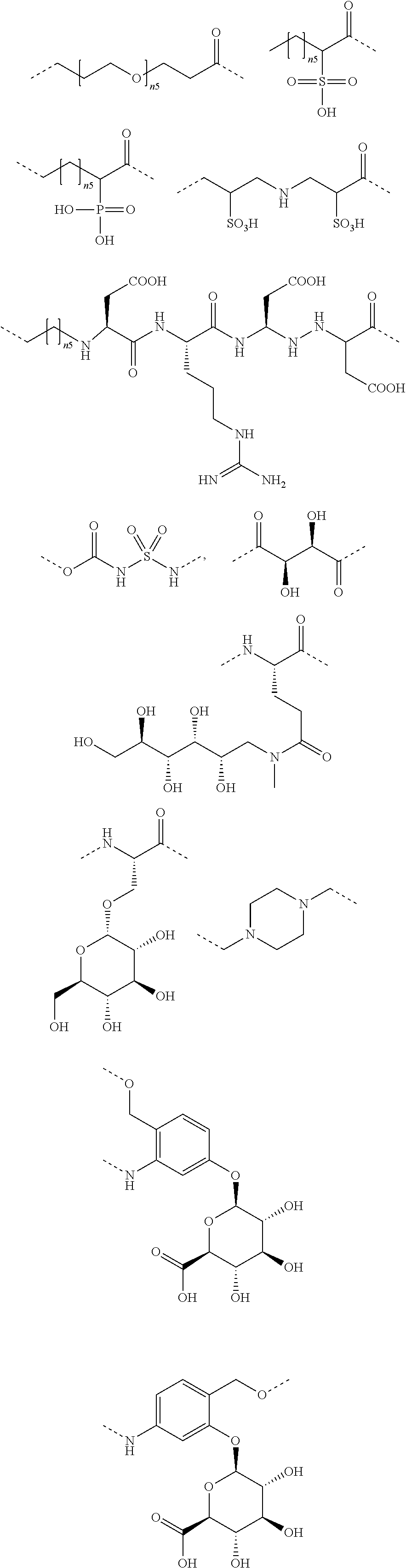
wherein each n5 is an integer selected from 1-30.

5. The conjugate of claim 1 or the pharmaceutically acceptable salt thereof, wherein
R_{P1} and R_{P2} are each independently selected from the group consisting of: 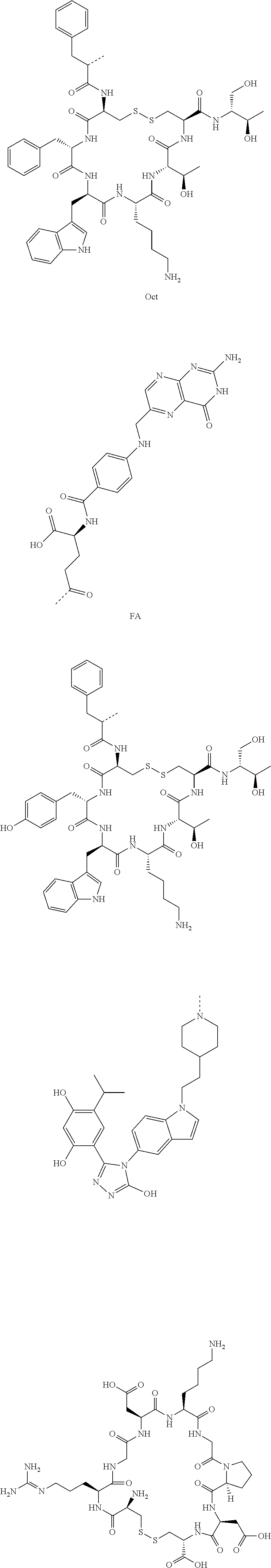
and/or,
R_{T} is selected from Table B1 or Table B2
TABLE B1

| | |
|---|---|
| 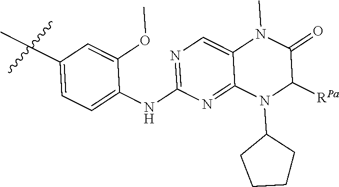 | P1 |
| 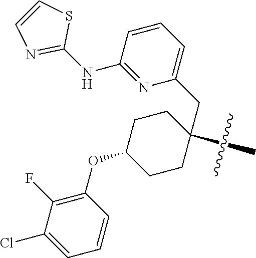 | P2 |
| 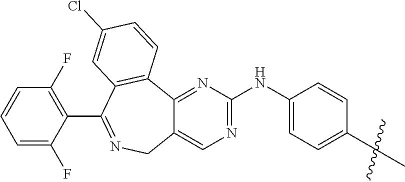 [SMILES: *c1ccc(Nc2ncc3c(n2)-c2ccc(Cl)cc2C(c2c(F)cccc2F)=NC3)cc1] | P3 |
| 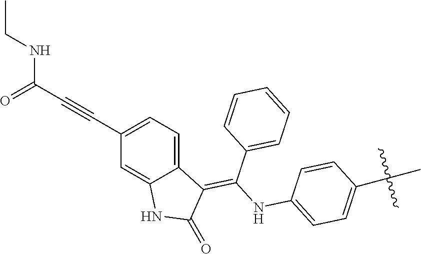 [SMILES: *c1ccc(N/C(=C2\C(=O)Nc3cc(C#CC(=O)NCC)ccc32)c2ccccc2)cc1] | P4 |

TABLE B2 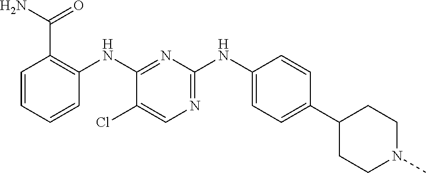 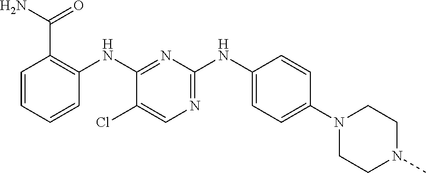 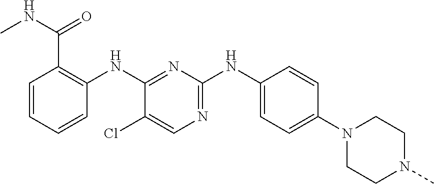
in each formula, R^{Pa} is selected from the group consisting of: optionally substituted C₁₋₆alkyl, optionally substituted C₂₋₆alkenyl, optionally substituted C₂₋₆alkynyl and/or,
R_{E} has a structure as shown in Formula A1 or A2: 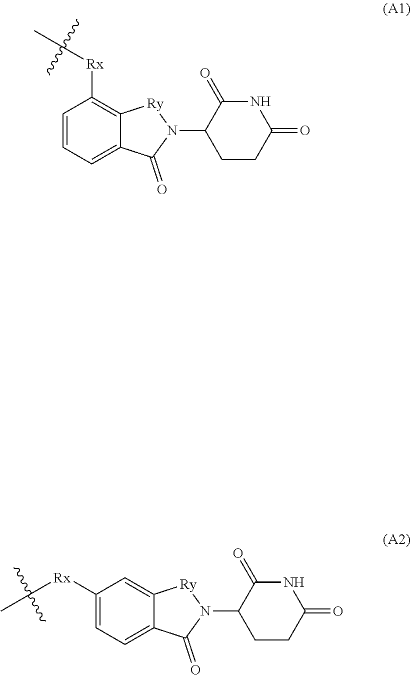
in formula A, R_{X} is selected from null, C1-C6 alkyl, C2-C6 alkenyl, C2-C6 alkynyl, O, NH, S, CO or SOₙ (n is 1 or 2) and the like; R_{Y} is CH₂, C═S, CO.

6. The conjugate of claim 1 or the pharmaceutically acceptable salt thereof, wherein the conjugate is selected from:

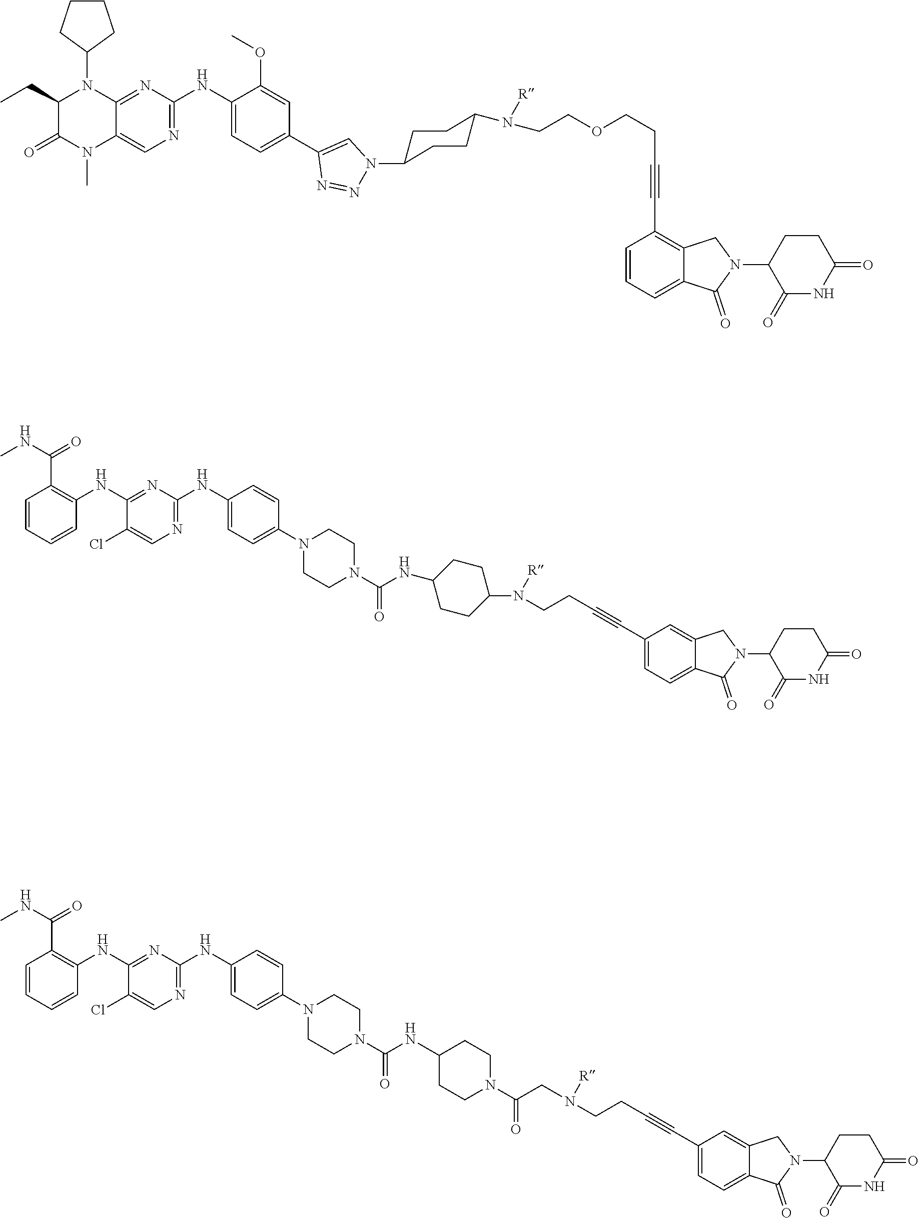

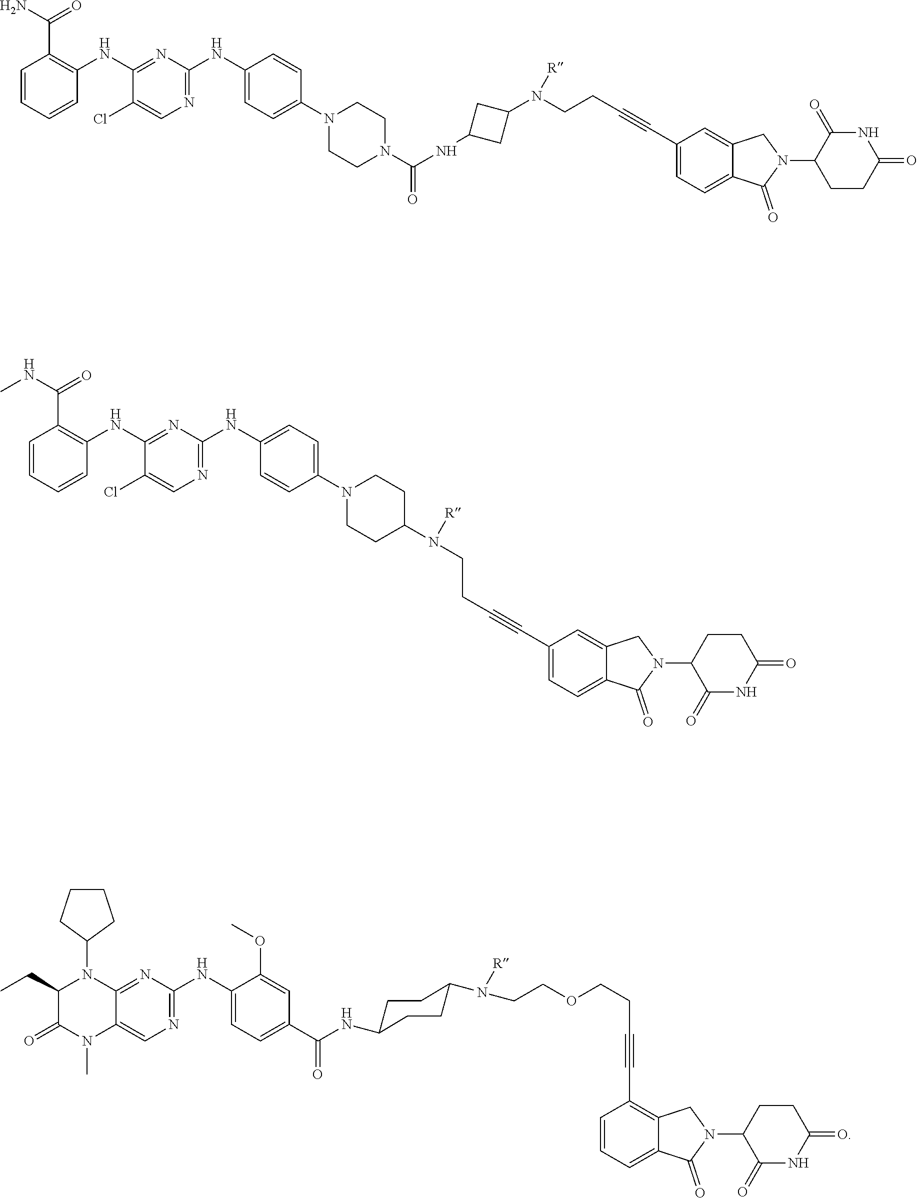

7. The conjugate of claim 1 or pharmaceutically acceptable salt thereof, wherein the conjugate is selected from Table D.

8. The conjugate of claim 1 or the pharmaceutically acceptable salt thereof, wherein L2 is L6, and L6 is of formula IIIa;
-(M)ₒ₁-(M^{N})-(M)ₒ₂- (IIIa)
wherein
M, and M^{N} are defined as above;
o1 and o2 are integers each independently selected from 1 to 50, and 4≤o1+o2≤49.

9. The conjugate of claim 1 or the pharmaceutically acceptable salt thereof, wherein the conjugate is selected from Table A2.

10. A pharmaceutical composition, wherein the pharmaceutical composition includes the conjugate of claim 1 or the pharmaceutically acceptable salt thereof and pharmaceutically acceptable carriers.

11. A method for treatment or prevention of diseases associated with excessive target protein wherein comprising step of administering the conjugate of claim 1 or the pharmaceutically acceptable salt thereof to a subject in need thereof.

12. (canceled)
